(19) United States
(12) Patent Application Publication
(10) Pub. No.: US 2018/0305334 A1
(43) Pub. Date: Oct. 25, 2018

(54) COMPOUNDS, COMPOSITIONS AND METHODS OF USE AGAINST STRESS GRANULES

(71) Applicant: Aquinnah Pharmaceuticals, Inc., Cambridge, MA (US)

(72) Inventors: Glenn R. Larsen, Sudbury, MA (US); Manfred Weigele, Cambridge, MA (US); Joseph P. Vacca, Telford, PA (US); Duane A. Burnett, Wayland, MA (US); Amy Ripka, Reading, MA (US)

(21) Appl. No.: 15/767,905

(22) PCT Filed: Oct. 14, 2016
(86) PCT No.: PCT/US16/57219

§ 371 Date: Apr. 12, 2018

Related U.S. Application Data
(60) Provisional application No. 62/241,653, filed on Oct. 14, 2015.

(51) Int. Cl. C07D 401/12; C07D 417/04; C07D 295/145; C07D 209/42; C07D 401/14; C07D 403/12; C07D 405/14; C07D 407/12; C07D 407/14; C07D 409/12; C07D 413/12; C07D 471/04; A61P 43/00

(52) U.S. Cl. CPC C07D 401/12; C07D 417/04; C07D 295/145; C07D 209/42; C07D 401/14; C07D 403/12; A61P 43/00; C07D 407/12; C07D 407/14; C07D 409/12; C07D 413/12; C07D 471/04; C07D 405/14

(57) ABSTRACT

Herein, compounds, compositions and methods for modulating inclusion formation and stress granules in cells related to the onset of neurodegenerative diseases, musculoskeletal diseases, cancer, ophthalmological diseases, and viral infections are described.

### FIELD OF THE INVENTION

The invention relates to compounds, compositions and methods for modulating inclusion formation and stress granules in cells, and for treatment of neurodegenerative diseases, musculoskeletal diseases, cancer, ophthalmological diseases, and viral infections.

### BACKGROUND OF THE INVENTION

One of the hallmarks of many neurodegenerative diseases is the accumulation of protein inclusions in the brain and central nervous system. These inclusions are insoluble aggregates of proteins and other cellular components that cause damage to cells and result in impaired function. Proteins such as tau, α-synuclein, huntingtin and β-amyloid have all been found to form inclusions in the brain and are linked to the development of a number of neurodegenerative diseases, including Alzheimer's disease and Huntington's disease. Recently, the TDP-43 protein was identified as one of the major components of protein inclusions that typify the neurogenerative diseases Amyotrophic Lateral Sclerosis (ALS) and Frontotemporal Lobar Dementia with ubiquitin inclusions (FTLD-U) (Ash, P. E., et al. (2010) Hum Mol Genet 19(16):3206-3218; Hanson, K. A., et al. (2010) J Biol Chem 285:11068-11072; Li, Y., et al. (2010) Proc Natl Acad Sci U.S.A. 107(7):3169-3174; Neumann, M., et al. (2006) Science 314:130-133; Tsai, K. J., et al. (2010) J Exp Med 207:1661-1673; Wils, H., et al. (2010) Proc Natl Acad Sci U.S.A. 170:3858-3863). Abnormalities in TDP-43 biology appear to be sufficient to cause neurodegenerative disease, as studies have indicated that mutations in TDP-43 occur in familial ALS (Barmada, S. J., et al. (2010) J Neurosci 30:639-649; Gitcho, M. A., et al. (2008) Ann Neurol 63(4): 535-538; Johnson, B. S., et al. (2009) J Biol Chem 284:20329-20339; Ling, S. C., et al. (2010) Proc Natl Acad Sci U.S.A. 107:13318-13323; Sreedharan, J., et al. (2008) Science 319:1668-1672). In addition, TDP-43 has been found to play a role in the stress granule machinery (Colombrita, C., et al. (2009) J Neurochem 111(4):1051-1061; Liu-Yesucevitz, L., et al. (2010) PLoS One 5(10):e13250). Analysis of the biology of the major proteins that accumulate in other neurodegenerative diseases has lead to major advances in our understanding of the pathophysiology of TDP-43 inclusions as well as the development of new drug discovery platforms.

Currently, it is believed that aggregates that accumulate in neurodegenerative diseases like ALS, FTLD-U, Parkinson's disease and Huntington's disease accumulate slowly and are very difficult to disaggregate or perhaps can't be disaggregated. Thus, there is a need in the art for compostions and methods that can rapidly disaggregate stress granules and/or inhabit their formation altogether.

### SUMMARY OF THE INVENTION

In one aspect, the invention provides a compound of Formula (I) or Formula (II):

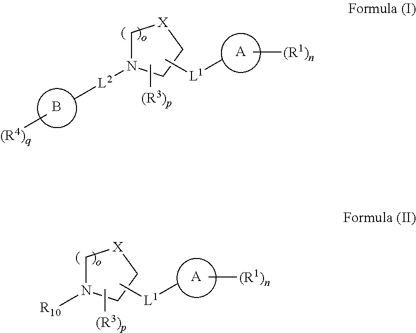

or a pharmaceutically acceptable salt thereof, wherein each of the variables above are described herein, for example, in the detailed description below.

In another aspect, the invention provides methods for treatment of a neurodegenerative disease or disorder, a musculoskeletal disease or disorder, a cancer, an ophthalmological disease or disorder (e.g., a retinal disease or disorder), and/or a viral infection in a subject, the method comprising administering a compound of Formula (I) or Formula (II) to a subject in need thereof.

In another aspect, the invention provides methods of diagnosing a neurodegenerative disease in a subject, the method comprising administering a compound of Formula (I) or Formula (II) to the subject. For use in diagnosis, the compound of Formula (I) or Formula (II) can be modified with a label.

In another aspect, the invention provides methods of modulating stress granules comprising contacting a cell with a compound of Formula (I) or Formula (II).

In another aspect, the invention provides methods of modulating TDP-43 inclusion formation comprising contacting a cell with a compound of Formula (I) or Formula (II).

In another aspect, the invention provides a method of screening for modulators of TDP-43 aggregation comprising contacting a compound of Formula (I) or Formula (II) with the cell that expresses TDP-43 and develops spontaneous inclusions.

Still other objects and advantages of the invention will become apparent to those of skill in the art from the disclosure herein, which is simply illustrative and not restrictive. Thus, other embodiments will be recognized by the skilled artisan without departing from the spirit and scope of the invention.

### BRIEF DESCRIPTION OF THE DRAWINGS

FIG. 1 is a table of exemplary compounds of the invention.

### DETAILED DESCRIPTION OF THE INVENTION

Amyotrophic lateral sclerosis (ALS), also known as Lou Gehrig's disease or Charcot disease, is a fatal neurodegenerative disease that occurs with an incidence of approximately 1/100,000 (Mitchell, J. D. and Borasio, G. D., (2007) Lancet 369:2031-41). There is currently no therapy for ALS, and the average survival rate of patients from the onset of the disease is roughly four years. ALS presents with motor weakness in the distal limbs that rapidly progresses proximally (Mitchell, J. D. and Borasio, G. D., (2007) Lancet 369:2031-41; Lambrechts, D. E., et al. (2004) Trends Mol Med 10:275-282). Studies over the past decade have indicated that TDP-43 is the major protein that accumulates in affected motor neurons in sporadic ALS (Neumann, M., et al. (2006) Science 314:130-133). The causes of sporadic ALS are not known, but identification of the major pathological species accumulating in the spinal cord of ALS patients represents a seminal advance for ALS research. To date, TDP-43 is the only protein that has been both genetically and pathologically linked with sporadic ALS, which represents the predominant form of the disease. Multiple papers have identified mutations in TDP-43 associated with sporadic and familial ALS (Sreedharan, J., et al. (2008) Science 319:1668-1672; Gitcho, M. A., et al. (2008) Ann Neurol 63(4):535-538; Neumann, M., et al. (2006) Science 314:130-133). Inhibitors of cell death and inclusions linked to TDP-43 represent a novel therapeutic approach to ALS, and may also elucidate the biochemical pathway linked to the formation of TDP-43 inclusions (Boyd, J. B., et al. (2014) J Biomol Screen 19(1):44-56). As such, TDP-43 represents one of the most promising targets for pharmacotherapy of ALS.

TDP-43 is a nuclear RNA binding protein that translocates to the cytoplasm in times of cellular stress, where it forms cytoplasmic inclusions. These inclusions then colocalize with reversible protein-mRNA aggregates termed “stress granules” (SGs) (Anderson P. and Kedersha, N. (2008) Trends Biochem Sci 33:141-150; Kedersha, N. and Anderson, P. (2002) Biochem Soc Trans 30:963-969; Lagier-Tourenne, C., et al. (2010) Hum Mol Genet 19:R46-R64). Under many stress-inducing conditions (e.g., arsenite treatment, nutrient deprivation), TDP-43 co-localization with SGs approaches 100%. The reversible nature of SG-based aggregation offers a biological pathway that can be applied to reverse the pathology and toxicity associated with TDP-43 inclusion formation. Studies show that agents that inhibit SG formation also inhibit formation of TDP-43 inclusions (Liu-Yesucevitz, L., et al. (2010) PLoS One 5(10):e13250). The relationship between TDP-43 and stress granules is important because it provides a novel approach for dispersing TDP-43 inclusions using physiological pathways that normally regulate this reversible process, rather than direct physical disruption of protein aggregation by a small molecule pharmaceutical. Investigating the particular elements of the SG pathway that regulate TDP-43 inclusion formation can identify selective approaches for therapeutic intervention to delay or halt the progression of disease. Stress granule biology also regulates autophagy and apoptosis, both of which are linked to neurodegeneration. Hence, compounds inhibiting TDP-43 aggregation may play a role in inhibiting neurodegeneration.

### Modulators of TDP-43 Inclusions and Stress Granules

Accordingly, in one aspect, the invention provides a compound of Formula (I):

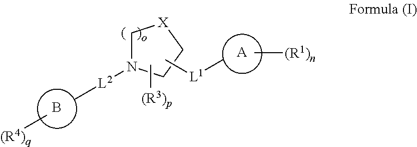

or a pharmaceutically acceptable salt thereof, wherein:

each of Ring A and Ring B is independently cycloalkyl, heterocyclyl, aryl, heteroaryl;

X is C(R′), C(R′)(R″), N, or NR^{A};

each of L¹ and L² is independently a bond, —C₁-C₆ alkyl-, —C₂-C₆ alkenyl-, —C₂-C₆ alkynyl-, —C₁-C₆ heteroalkyl-, —C(O)—, —OC(O)—, —C(O)O—, —OC(O)O—, —C(O)NR^{A}—, —NR^{A}C(O)—, —C(O)NR^{A}—C₁-C₆ alkyl, —C₁-C₆ alkyl-C(O)NR^{A}—, —NR^{A}C(O)—C₁-C₆ alkyl-, —C₁-C₆ alkyl-NR^{A}C(O)—, —C(O)NR^{A}—C₁-C₆ heteroalkyl-, —C₁-C₆ heteroalkyl-C(O)NR^{A}—, —NR^{A}C(O)—C₁-C₆ heteroalkyl-, —C₁-C₆ heteroalkyl-NR^{A}C(O)—, —C₁-C₆ alkyl-C(O)—, —C(O)—C₁-C₆ alkyl, —C₁-C₆ heteroalkyl-C(O)—, —C(O)—C₁-C₆ heteroalkyl, —C(O)—C₁-C₆ alkyl-C(O)NR^{A}—, —S(O)ₓ—, —OS(O)ₓ, —C(O)NR^{A}S(O)ₓ—, —NR^{A}S(O)ₓ—, or —S(O)ₓNR^{A}—, each of which is optionally substituted with 1-5 R⁵;

each of R¹ and R⁴ is independently C₁-C₆ alkyl, C₂-C₆ alkenyl, C₂-C₆ alkynyl, C₁-C₆ heteroalkyl, C₁-C₆ haloalkyl, halo, cyano, cycloalkyl, heterocyclyl, aryl, heteroaryl, —OR^{B}, —NR^{A}R^{C}, —NR^{A}C(O)R^{D}, —S(O)ₓR^{E}, —OS(O)ₓR^{E}, —C(O)NR^{A}S(O)ₓR^{E}, —NR^{A}S(O)ₓR^{E}, or —S(O)ₓNR^{A}, each of which is optionally substituted with 1-5 R⁶;

R³ is H, C₁-C₆ alkyl, C₂-C₆ alkenyl, C₂-C₆ alkynyl, C₁-C₆ heteroalkyl, C₁-C₆ haloalkyl, halo, cyano, nitro, cycloalkyl, heterocyclyl, aryl, heteroaryl, —OR^{B}, —NR^{A}R^{C}, —C(O)R^{D}, —C(O)OR^{B}, —C(O)NR^{A}R^{C}, —NR^{A}C(O)R^{D}, —NR^{A}C(O)NR^{B}R^{C}, —SR^{E}, —S(O)ₓR^{E}, —NR^{A}S(O)ₓR^{E}, or —S(O)ₓNR^{A}R^{C}, each of which is optionally substituted with 1-5 R⁷; or or two R³, taken together with the atoms to which they are attached, form a ring (e.g., a 5-7 membered ring), optionally substituted with 1-5 R⁷;

each of R′ and R″ is independently H, C₁-C₆ alkyl, C₁-C₆ heteroalkyl, C₁-C₆ haloalkyl, halo, cyano, cycloalkyl, or heterocyclyl, each of which is optionally substituted with 1-5 R⁷;

each of R⁵, R⁶, and R⁷ is independently C₁-C₆ alkyl, C₂-C₆ alkenyl, C₂-C₆ alkynyl, C₁-C₆ heteroalkyl, C₁-C₆ haloalkyl, halo, cyano, cycloalkyl, heterocyclyl, aryl, heteroaryl, —OR^{B}, —C(O)R^{D}, —C(O)OR^{B}, —C(O)NR^{A}R^{C}, or —SR^{E}, each of which is optionally substituted with 1-5 R⁸;

each R^{A}, R^{B}, R^{C}, R^{D}, or R^{E} is independently H, C₁-C₆ alkyl, C₁-C₆ heteroalkyl, C₁-C₆ haloalkyl, cycloalkyl, heterocyclyl, aryl, heteroaryl, arylalkyl, heteroarylalkyl, cycloalkylalkyl, or heterocycloalkyl, each of which is optionally substituted with 1-4 R⁸;

or R^{A} and R^{C}, together with the atoms to which each is attached, form a heterocyclyl ring optionally substituted with 1-4 R⁸;

each R⁸ is independently C₁-C₆ alkyl, C₂-C₆ alkenyl, C₂-C₆ alkynyl, C₁-C₆ heteroalkyl, C₁-C₆ haloalkyl, cycloalkyl, heterocyclyl, aryl, heteroaryl, halo, cyano, or nitro, each of which is optionally substituted with 1-5 R⁹;

each R⁹ is C₁-C₆ alkyl, halo, hydroxy, cycloalkyl, alkoxy, keto, cyano, or nitro;

each of n and q is independently 0, 1, 2, 3, 4, 5, or 6;

o is 1, 2, or 3;

p is 0, 1, 2, 3 or 4; and

x is 0, 1, or 2;

wherein when L¹ is connected to X, X is C(R′) or N.

In some embodiments, Ring A is aryl (e.g., monocyclic or bicyclic aryl). In some embodiments, Ring A is phenyl

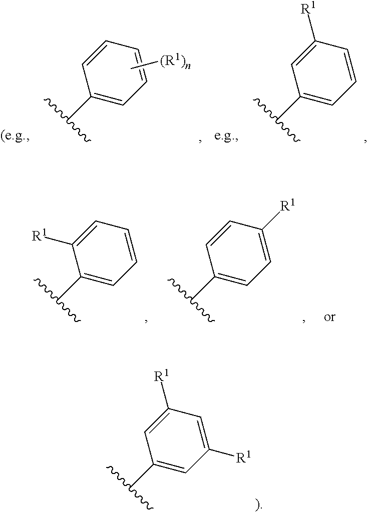

In some embodiments, Ring A is naphthyl

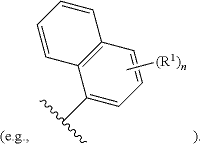

In some embodiments, R¹ is C₁-C₆ alkyl (e.g., methyl or ethyl), halo (e.g., fluoro or chloro), cyano, or —OR^{B} (e.g., —OCH₃, OCF₃, OCHF₂). In some embodiments, R¹ is —OR^{B}, (e.g., —OCH₃, OCF₃, OCHF₂). In some embodiments, n is 1 or 2.

In some embodiments, Ring A is heteroaryl. In some embodiments, Ring A is a bicyclic heteroaryl (e.g., a bicyclic nitrogen-containing heteroaryl, a bicyclic sulfur-containing heteroaryl, or a bicyclic oxygen-containing heteroaryl). In some embodiments, Ring A is indolyl, indolinyl, indazolyl, benzofuranyl, benzoimidazolyl, benzooxazolyl, or benzothiazolyl (e.g.,

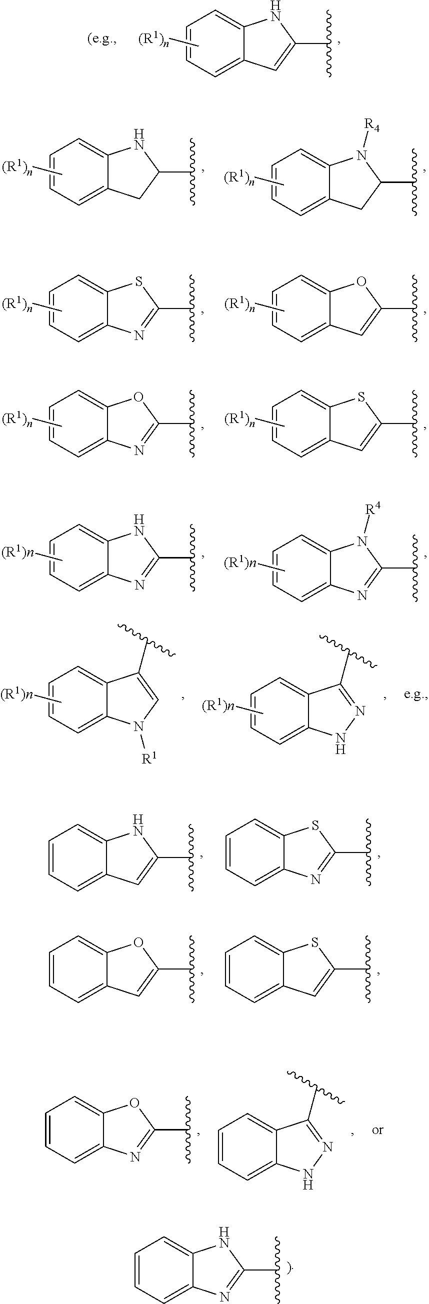

In some embodiments, n is 0.

In some embodiments, n is 1, 2, or 3. In some embodiments, n is 1 or 2. In some embodiments, n is 1.

In some embodiments, R¹ is C₁-C₆ alkyl (e.g., methyl or ethyl), halo (e.g., fluoro or chloro), cyano, or —OR^{B} (e.g., —OCH₃, OCF₃, OCHF₂, —OCH₂-aryl). In some embodiments, R¹ is —OR^{B}, (e.g., —OCH₃, OCF₃, OCHF₂).

In some embodiments, Ring A is a monocyclic heteroaryl (e.g., a monocyclic nitrogen-containing heteroaryl or monocyclic oxygen-containing heteroaryl). In some embodiments, Ring A is a 5-membered heteroaryl or a 6-membered heteroaryl. In some embodiments, Ring A is pyrrolyl, furanyl, or pyridyl,

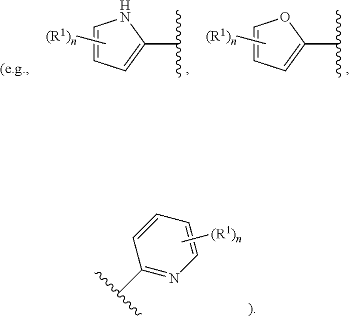

In some embodiments, Ring B is aryl (e.g., monocyclic aryl or bicyclic aryl). In some embodiments, Ring B is phenyl,

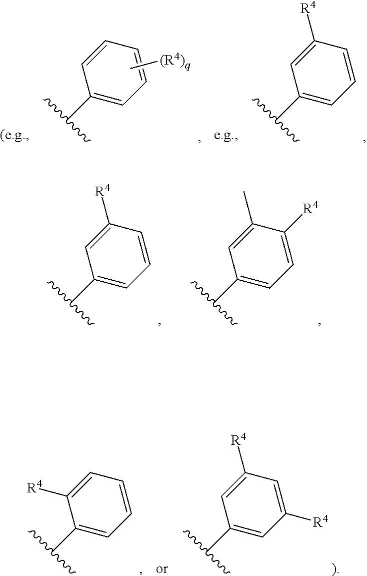

In some embodiments, Ring B is naphthyl (e.g.,

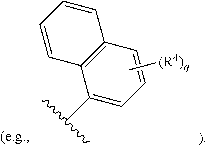

In some embodiments, Ring B is cycloalkyl (e.g., monocyclic or bicyclic cycloalkyl). In some embodiments, Ring B is bicyclic cycloalkyl

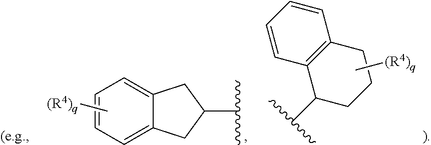

In some embodiments, Ring B is heteroaryl. In some embodiments, Ring B is a bicyclic heteroaryl (e.g., a bicyclic nitrogen-containing heteroaryl). In some embodiments, Ring B is indolyl, indolinyl, indazolyl, benzofuranyl, benzoimidazolyl, benzooxazolyl, or benzothiazolyl

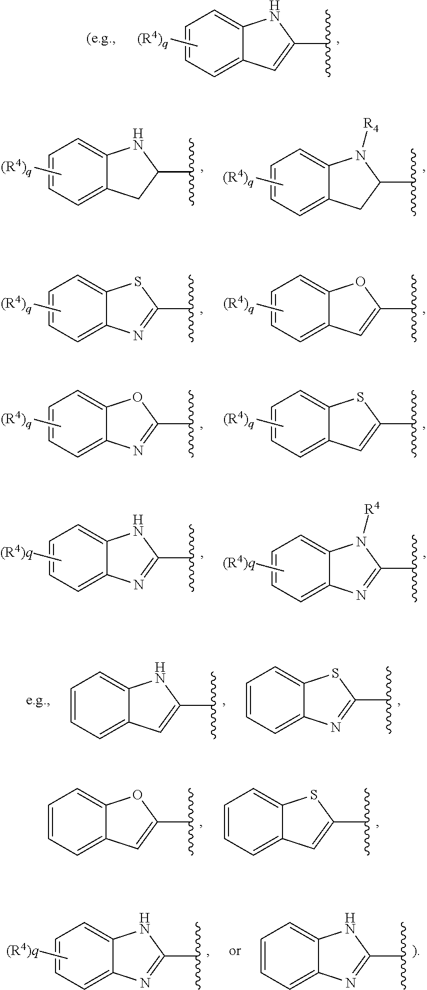

In some embodiments, Ring B is a monocyclic heteroaryl (e.g., a monocyclic nitrogen-containing heteroaryl). In some embodiments, Ring B is pyrrolyl

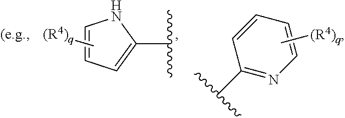

In some embodiments, Ring B is heterocyclyl. In some embodiments, Ring B is a nitrogen-containing heterocyclyl or oxygen-containing heterocyclyl (e.g., tetrahydropyranyl,

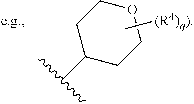

In some embodiments, q is 0.

In some embodiments, q is 1, 2, or 3. In some embodiments, q is 1 or 2. In some embodiments, q is 1. In some embodiments, q is 2.

In some embodiments, R⁴ is C₁-C₆ alkyl (e.g., methyl or ethyl), halo (e.g., fluoro or chloro), cyano, —C(O)OR^{B} (e.g., —C(O)OH or —C(O)OCH₃), or —OR^{B} (e.g., —OCH₃, OCF₃, OCHF₂, —OCH₂-aryl). In some embodiments, R⁴ is —OR^{B}, (e.g., —OCH₃, OCF₃, OCHF₂, —OCH₂-aryl).

In some embodiments, X is C(R′)(R″). In some embodiments, each of R′ and R″ is independently H, C₁-C₆ alkyl, or halo. In some embodiments, each of R′ and R″ is independently H.

In some embodiments, when L¹ is connected to X, X is C(R′). In some embodiments, R′ is H. In some embodiments, when L¹ is connected to X, X is N.

In some embodiments, X is NR^{A}. In some embodiments, R^{A} is H, C₁-C₆ alkyl (methyl, ethyl, isopropyl), or C₁-C₆ heteroalkyl.

In some embodiments, each of L¹ and L² is independently a bond, C₁-C₆ alkyl, C₁-C₆ heteroalkyl, —C(O)—, —C(O)NR^{A}—, —NR^{A}C(O)—, —C(O)NR^{A}—C₁-C₆ alkyl, —NR^{A}C(O)—C₁-C₆ alkyl, —NR^{A}C(O)—C₁-C₆ heteroalkyl, —C(O)—C₁-C₆ alkyl, C₁-C₆ alkyl-C(O)—, C₁-C₆ alkyl-NR^{A}C(O)—, —S(O)ₓ—, —OS(O)ₓ, —C(O)NR^{A}S(O)ₓ—, —NR^{A}S(O)ₓ—, or —S(O)ₓNR^{A}—, each of which is optionally substituted with 1-5 R⁵. In some embodiments, each of L¹ and L² is independently a bond, C₁-C₆ alkyl, —C(O)—, —C(O)NR^{A}—C₁-C₆ alkyl, —C(O)—C₁-C₆ alkyl, or —S(O)ₓ—, each of which is optionally substituted with 1-5 R⁵.

In some embodiments, L¹ and L² is independently a bond. In some embodiments, one of L¹ and L² is independently C₁-C₆ alkyl (e.g., CH₂, CH₂CH₂). In some embodiments, one of L¹ and L² is independently C₁-C₆ alkyl-NR^{A}C(O)—, optionally substituted with 1-5 R⁵. In some embodiments, one of L¹ and L² is independently —NR^{A}C(O)—C₁-C₆ heteroalkyl, optionally substituted with 1-5 R⁵.

In some embodiments, L¹ is C₁-C₆ alkyl or C₁-C₆ alkyl-NR^{A}C(O)—. In some embodiments, L¹ is C₁-C₆ alkyl-NR^{A}C(O)— (e.g., CH₂—NR^{A}C(O)—). In some embodiments, L¹ is —CH₂—N(CH₂CH₃)R^{A}C(O)—. In some embodiments, R^{A} is H, C₁-C₆ alkyl (e.g., methyl, ethyl, isopropyl), C₁-C₆ heteroalkyl, C₁-C₆ haloalkyl (e.g., CH₂CF₃), cycloalkyl (e.g., cyclohexyl), aryl (e.g., phenyl), cycloalkylalkyl, or arylalkyl (e.g., CH₂-phenyl). In some embodiments, R^{A} is H.

In some embodiments, L² is a bond, C₁-C₆ alkyl (e.g., methyl or ethyl), —S(O)ₓ— (e.g., S(O)₂), or —C(O)—C₁-C₆ alkyl (e.g., —C(O)CH₂—), each of which is optionally substituted with 1-5 R⁵. In some embodiments, L² is C₁-C₆ alkyl (e.g., methyl or ethyl).

In some embodiments, R⁵ is C₁-C₆ alkyl (e.g., methyl or ethyl), C₁-C₆ haloalkyl (e.g., CF₃), cycloalkyl (e.g., cyclopropyl), or halo (e.g., fluoro or chloro).

In some embodiments, p is 0, 1, or 2. In some embodiments, p is 0.

In some embodiments, p is 1 or 2. In some embodiments, p is 2, and each R³ is C₁-C₆ alkyl (e.g., methyl or ethyl). In some embodiments, p is 2, and each R³ is C₁-C₆ alkyl (e.g., methyl or ethyl), wherein both R³ is joined together to form a 6- or 7-membered ring.

In some embodiments, o is 1 or 2. In some embodiments, o is 1. In some embodiments, o is 2.

In some embodiments, the compound of Formula (I) is not

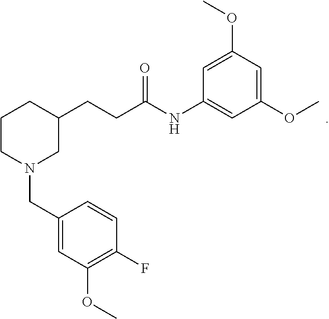 [SMILES: COc1cc(NC(=O)CCC2CCCN(Cc3ccc(F)c(OC)c3)C2)cc(OC)c1]

In some embodiments, the compound of Formula (I) is a compound of Formula (I-a), Formula (I-b), or Formula (I-c):

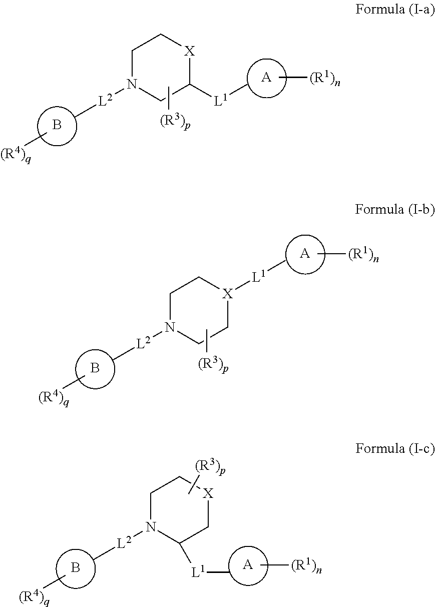

or a pharmaceutically acceptable salt thereof, wherein:

each of Ring A and Ring B is independently aryl or heteroaryl;

X is C(R′)(R″) or NR^{A};

each of L¹ and L² is independently a bond, C₁-C₆ alkyl, C₂-C₆ alkenyl, C₂-C₆ alkynyl, C₁-C₆ heteroalkyl, —C(O)—, —OC(O)—, —C(O)O—, —OC(O)O—, —C(O)NR^{A}—, —NR^{A}C(O)—, —C(O)NR^{A}—C₁-C₆ alkyl, —NR^{A}C(O)—C₁-C₆ alkyl, —NR^{A}C(O)—C₁-C₆ heteroalkyl, C₁-C₆ alkyl-C(O)—, C₁-C₆ heteroalkyl-C(O)—, —C(O)—C₁-C₆ alkyl, —C(O)—C₁-C₆ alkyl-C(O)NR^{A}—, or C₁-C₆ alkyl-NR^{A}C(O)—, each of which is optionally substituted with 1-5 R⁵;

each of R¹ and R⁴ is independently C₁-C₆ alkyl, C₂-C₆ alkenyl, C₂-C₆ alkynyl, C₁-C₆ heteroalkyl, C₁-C₆ haloalkyl, halo, cyano, cycloalkyl, heterocyclyl, —OR^{B}, —NR^{A}R^{C}, —NR^{A}C(O)R^{D}, or —SR^{E}, each of which is optionally substituted with 1-5 R⁶;

R³ is H, C₁-C₆ alkyl, C₂-C₆ alkenyl, C₂-C₆ alkynyl, C₁-C₆ heteroalkyl, C₁-C₆ haloalkyl, halo, cyano, nitro, cycloalkyl, heterocyclyl, aryl, heteroaryl, —OR^{B}, —NR^{A}R^{C}, —C(O)R^{D}, —C(O)OR^{B}, —C(O)NR^{A}R^{C}, —NR^{A}C(O)R^{D}, —NR^{A}C(O)NR^{B}R^{C}, —SR^{E}, —S(O)R^{E}, —S(O)₂R^{E}, —NR^{A}S(O)₂R^{E}, or —S(O)₂NR^{A}R^{C}, each of which is optionally substituted with 1-5 R⁷;

each of R′ and R″ is independently H, C₁-C₆ alkyl, C₁-C₆ heteroalkyl, C₁-C₆ haloalkyl, halo, cyano, cycloalkyl, or heterocyclyl, each of which is optionally substituted with 1-5 R⁷;

each of R⁵, R⁶, and R⁷ is independently C₁-C₆ alkyl, C₂-C₆ alkenyl, C₂-C₆ alkynyl, C₁-C₆ heteroalkyl, C₁-C₆ haloalkyl, halo, cyano, cycloalkyl, heterocyclyl, —C(O)R^{D}, —C(O)OR^{B}, —C(O)NR^{A}R^{C}, —OR^{B}, or —SR^{E}, each of which is optionally substituted with 1-5 R⁸;

each R^{A}, R^{B}, R^{C}, R^{D}, or R^{E} is independently H, C₁-C₆ alkyl, C₁-C₆ heteroalkyl, C₁-C₆ haloalkyl, cycloalkyl, heterocyclyl, aryl, heteroaryl, arylalkyl, heteroarylalkyl, cycloalkylalkyl, or heterocycloalkyl, each of which is optionally substituted with 1-4 occurrences of R⁸; or R^{A} and R^{C}, together with the atoms to which each is attached, form a heterocyclyl ring optionally substituted with 1-4 R⁸;

each R⁸ is independently C₁-C₆ alkyl, C₂-C₆ alkenyl, C₂-C₆ alkynyl, C₁-C₆ heteroalkyl, C₁-C₆ haloalkyl, cycloalkyl, heterocyclyl, aryl, heteroaryl, halo, cyano, or nitro, each of which is optionally substituted with 1-5 R⁹;

each R⁹ is C₁-C₆ alkyl, halo, hydroxy, cycloalkyl, alkoxy, keto, cyano, or nitro;

each of n and q is independently 0, 1, 2, 3, or 4; and

p is 0, 1, 2, 3 or 4;

provided the compound is not

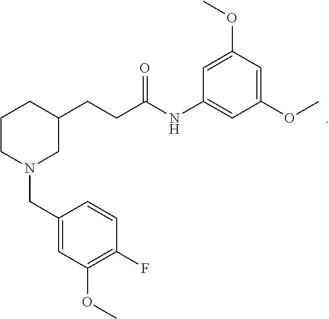 [SMILES: COc1cc(NC(=O)CCC2CCCN(Cc3ccc(F)c(OC)c3)C2)cc(OC)c1]

In some embodiments, the compound of Formula (I) is a compound of Formula (I-d), Formula (I-e), or Formula (I-f):

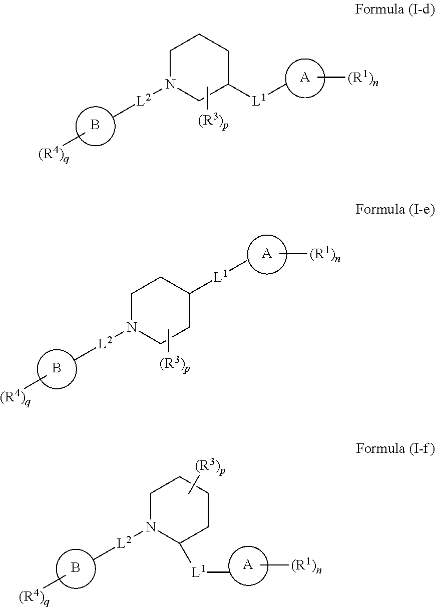

or a pharmaceutically acceptable salt thereof, wherein Ring A, Ring B, L¹, L², R¹, R³, R⁴, n, p, q, and subvariables thereof are as described for Formula (I).

In some embodiments, the compound of Formula (I) is a compound of Formula (I-g), Formula (I-h), or Formula (I-i):

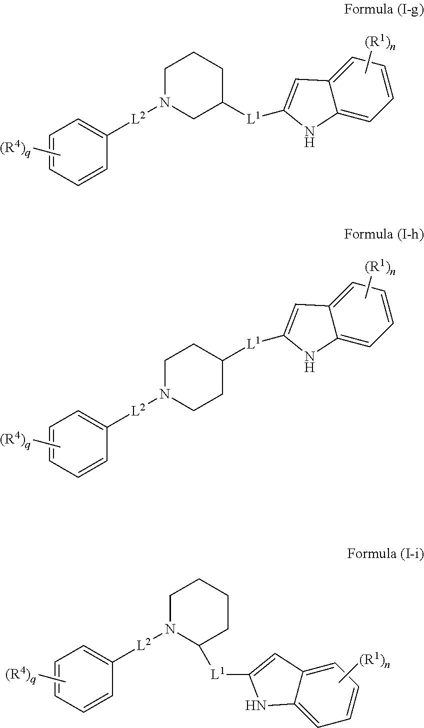

or a pharmaceutically acceptable salt thereof, wherein L¹, L², R¹, R⁴, n, q, and subvariables thereof are as described for Formula (I).

In some embodiments, the compound of Formula (I) is a compound of Formula (I-j):

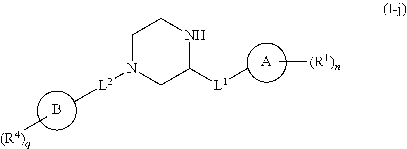

or a pharmaceutically acceptable salt thereof, wherein Ring A, Ring B, L¹, L², R¹, R⁴, n, q, and subvariables thereof are described as for Formula (I).

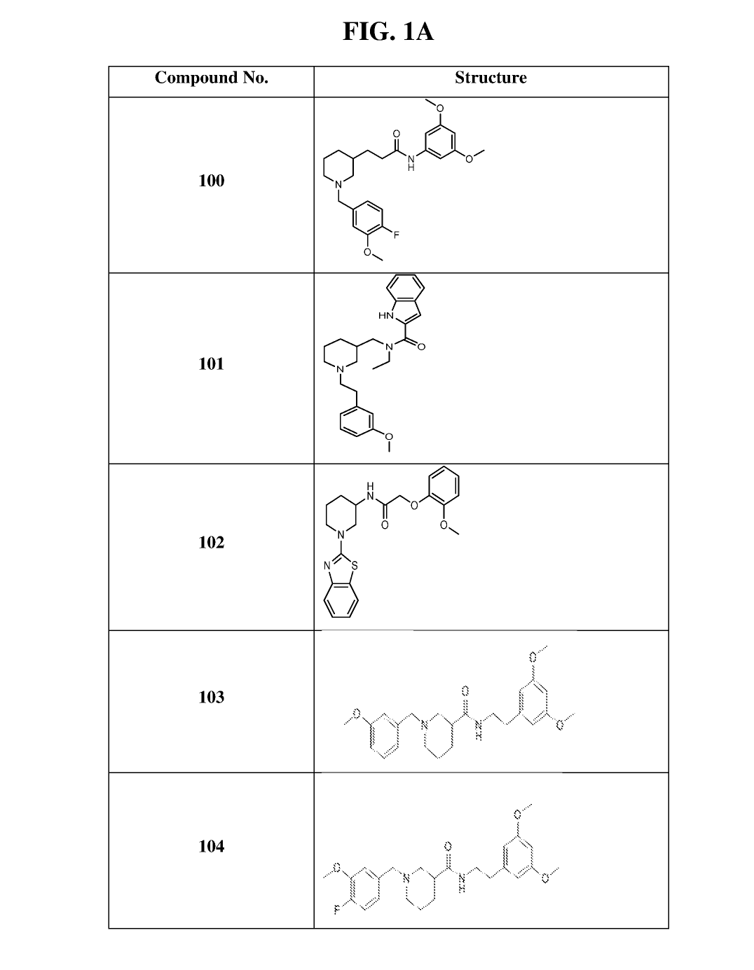

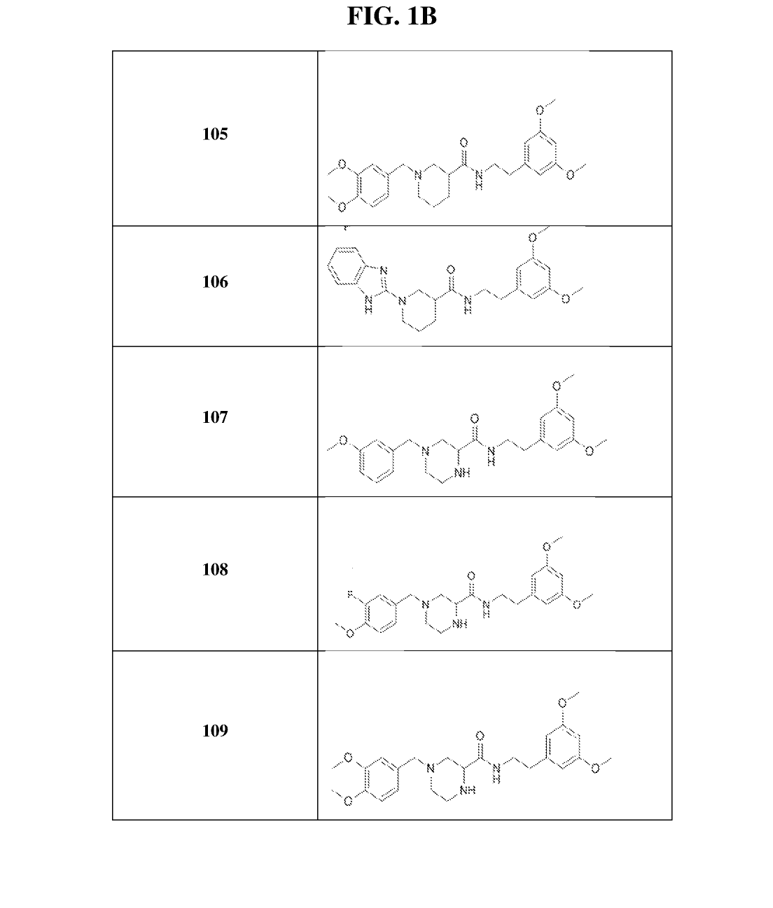

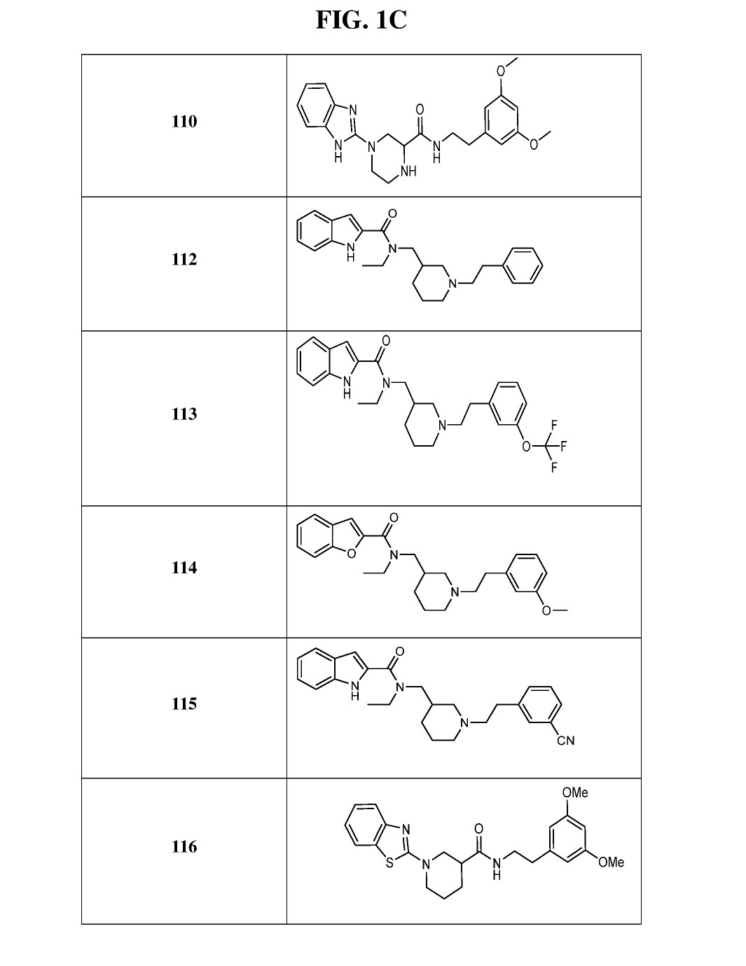

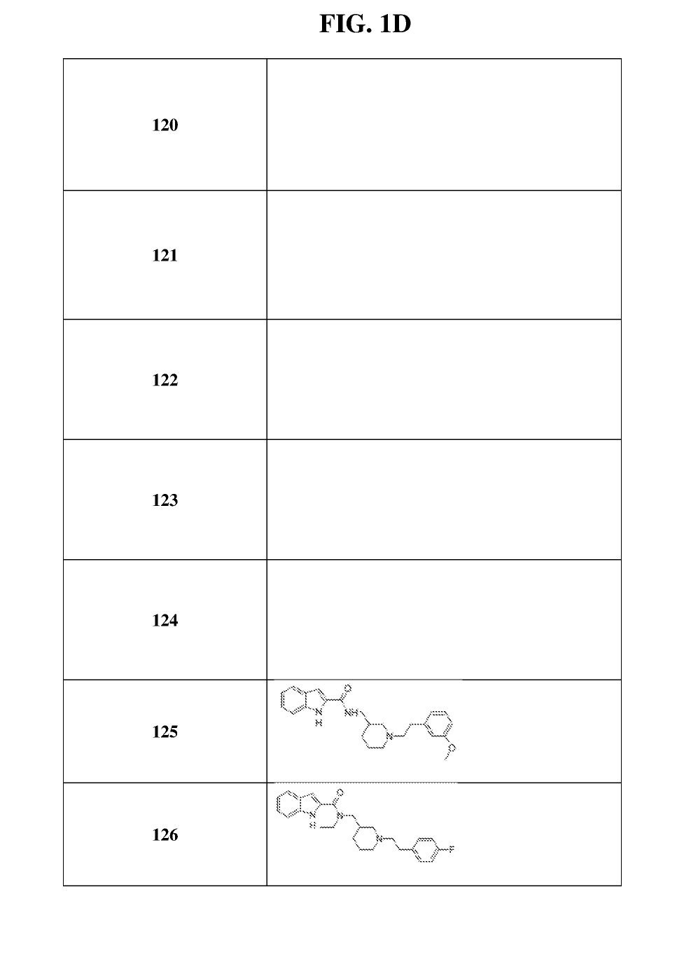

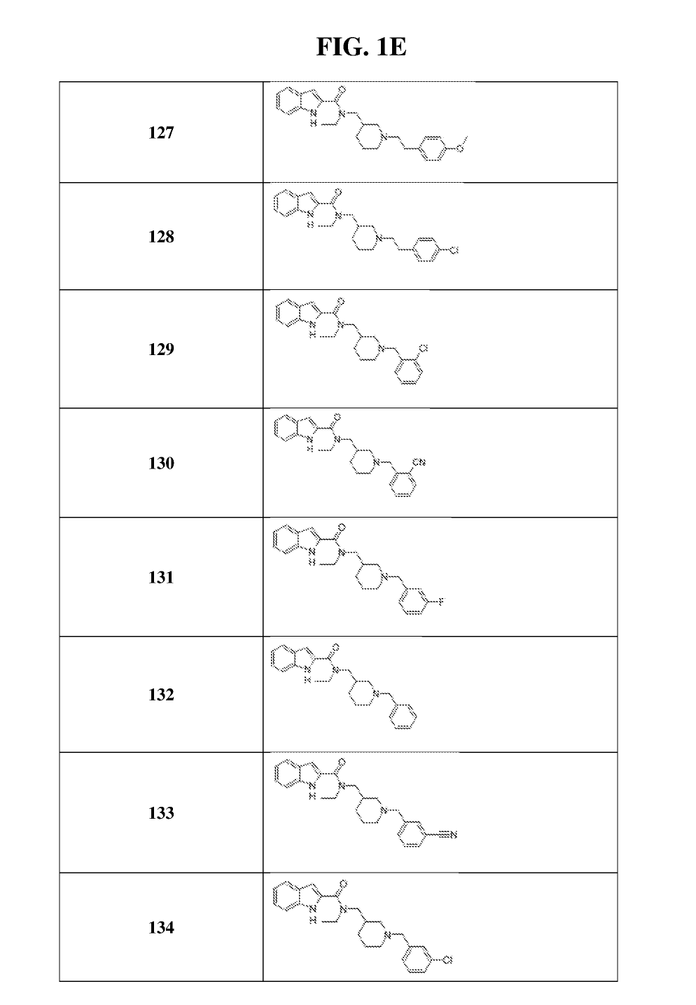

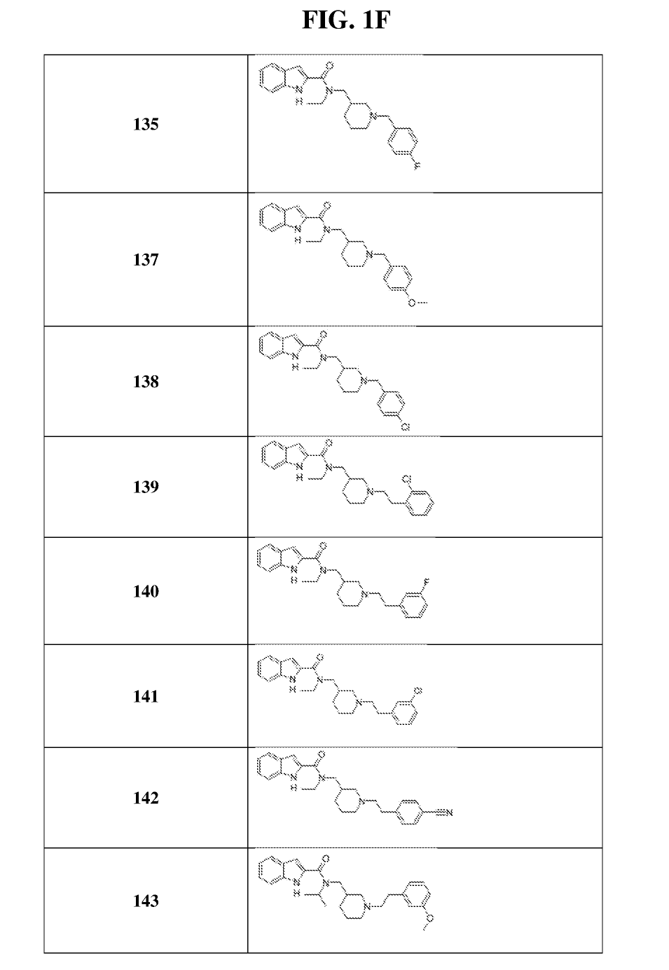

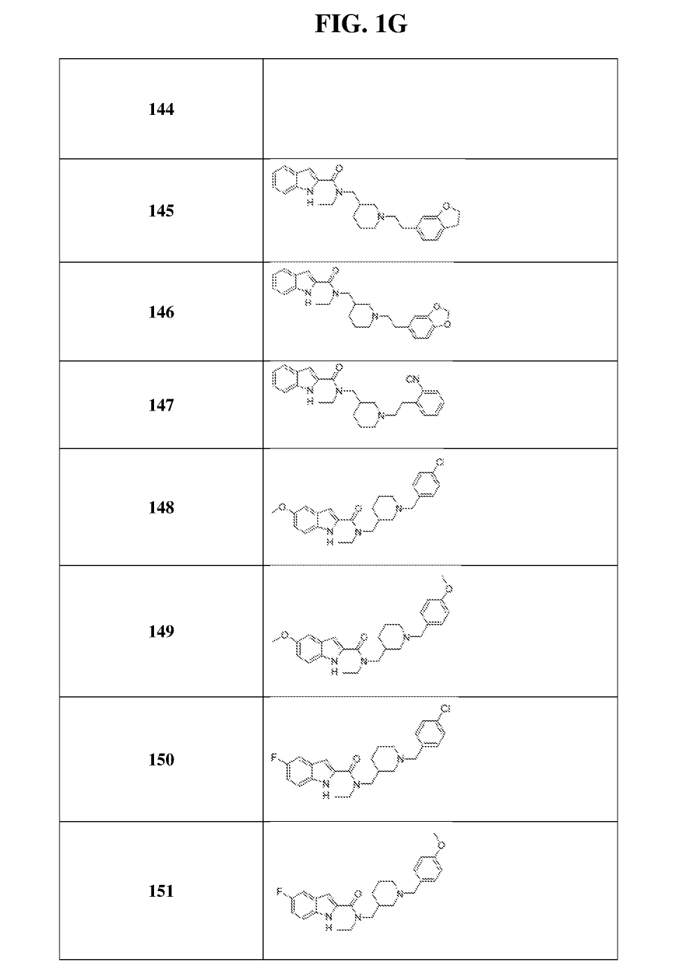

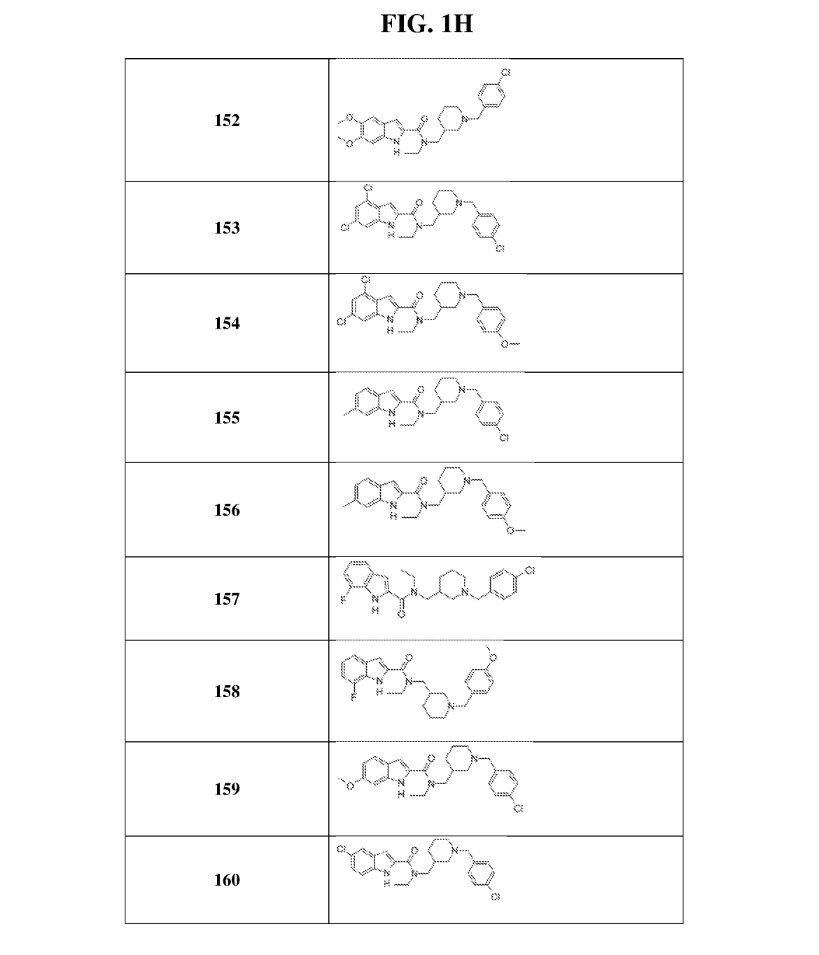

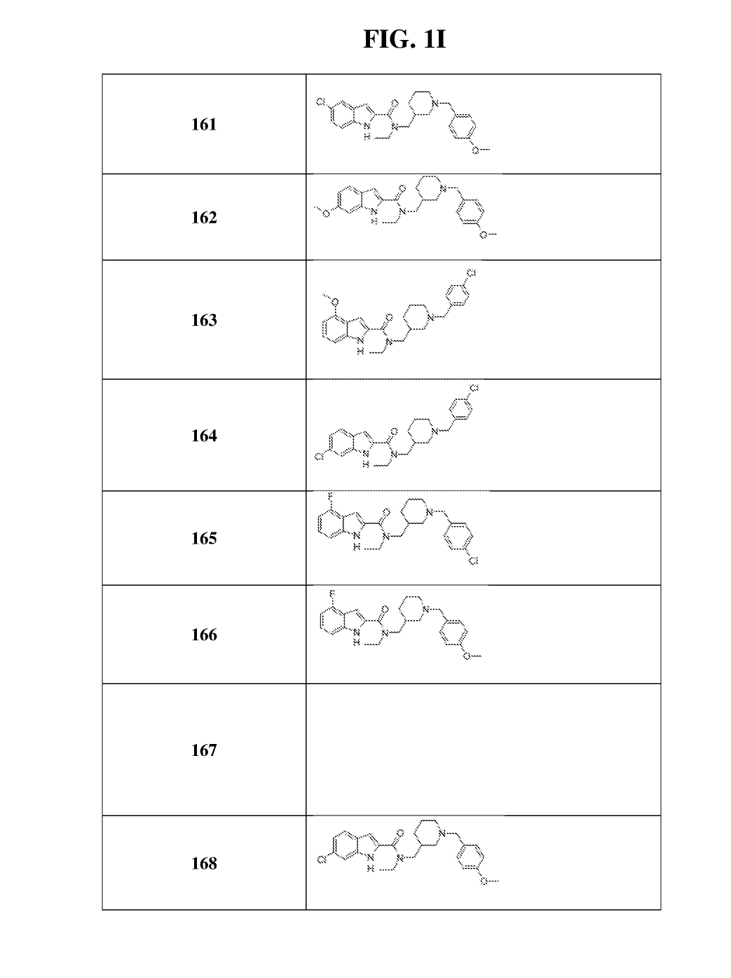

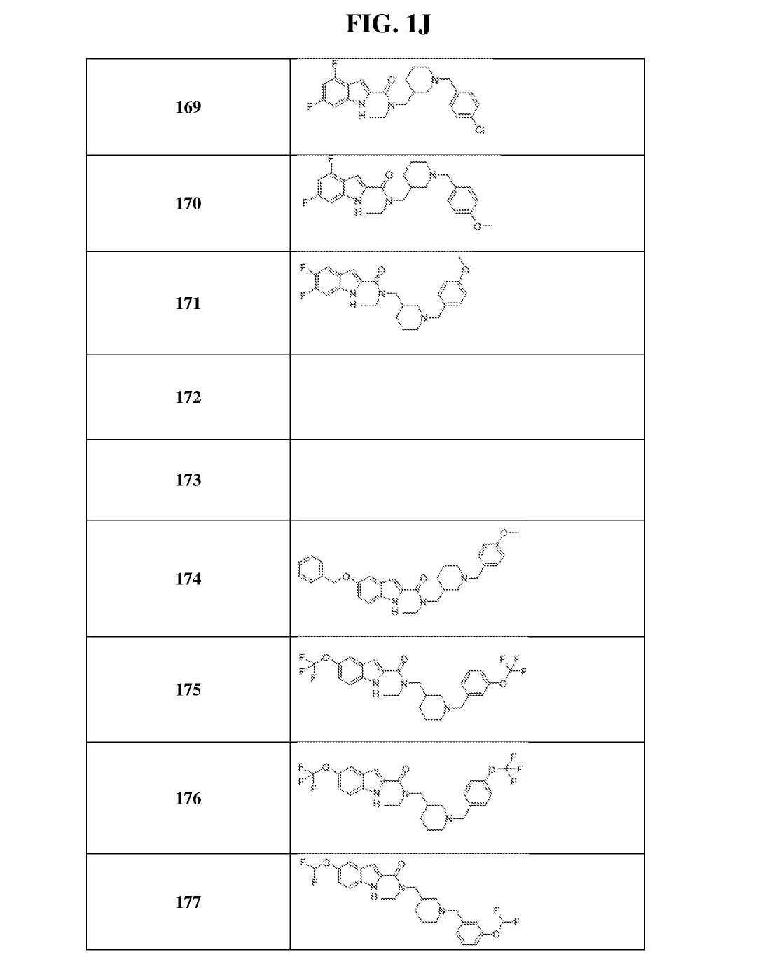

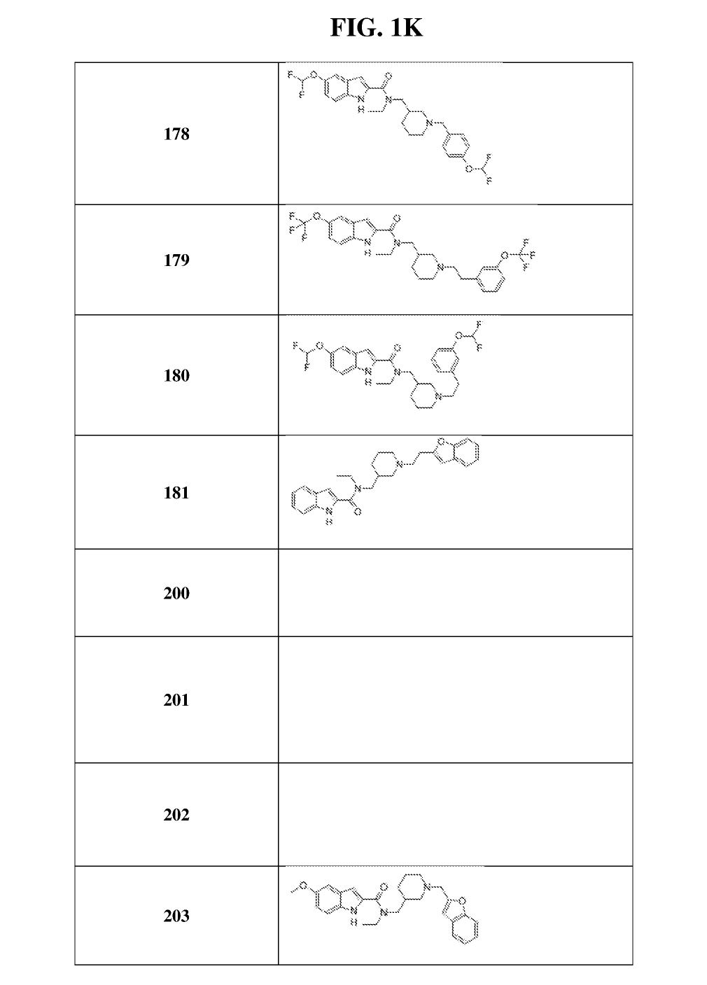

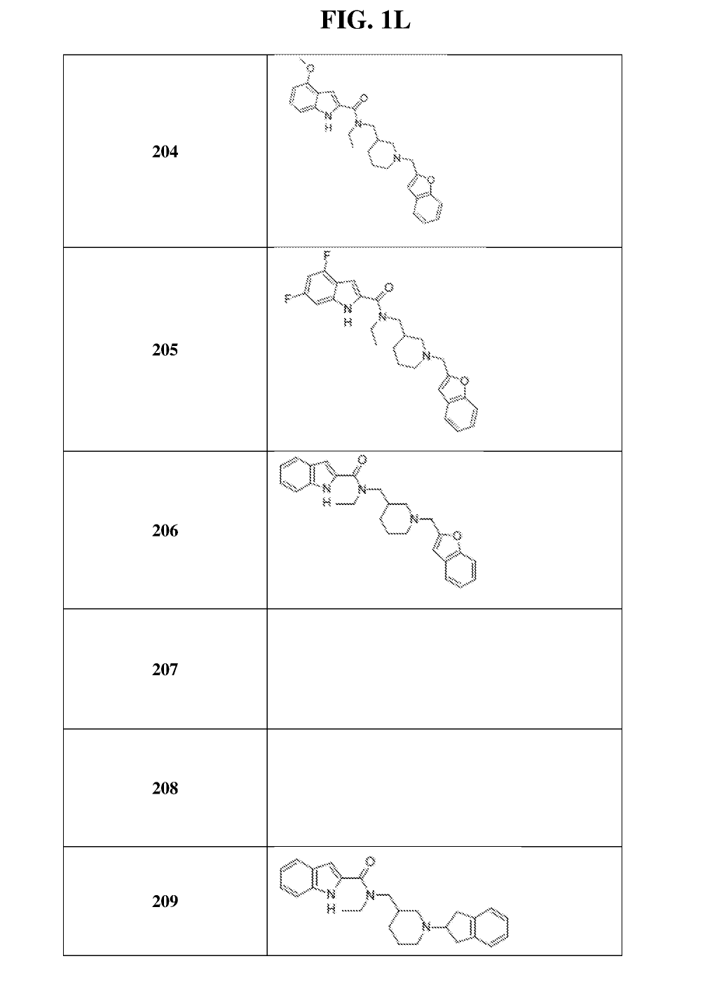

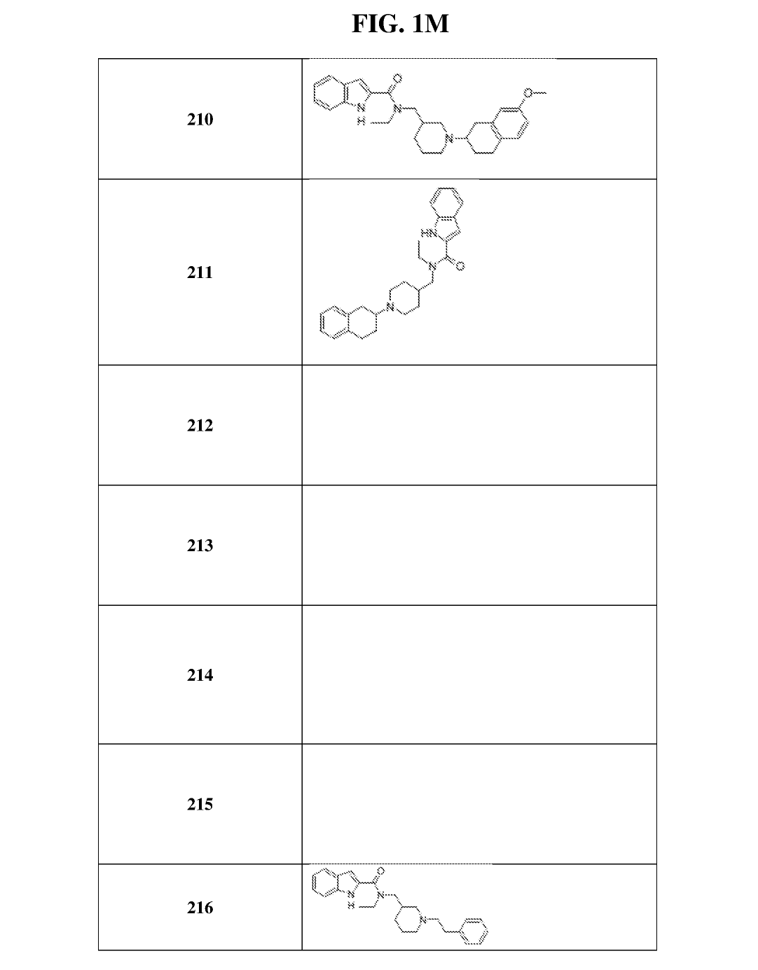

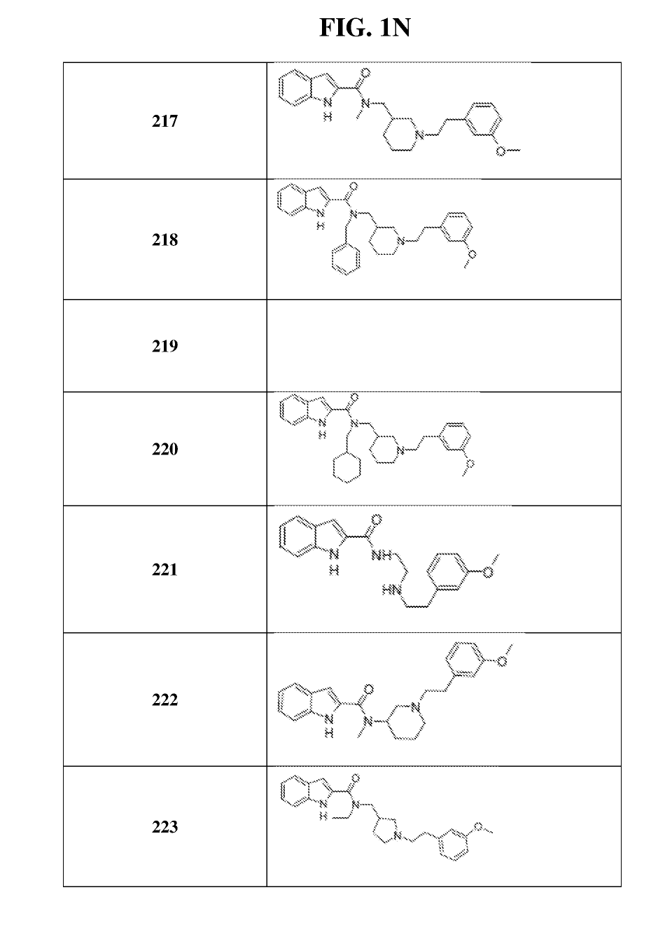

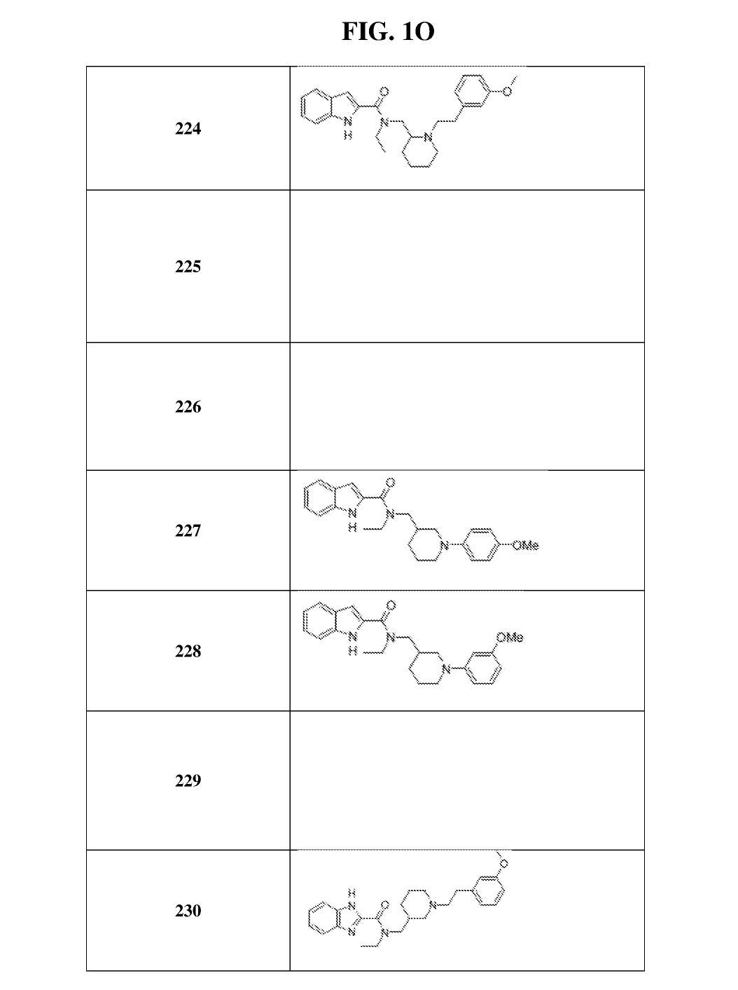

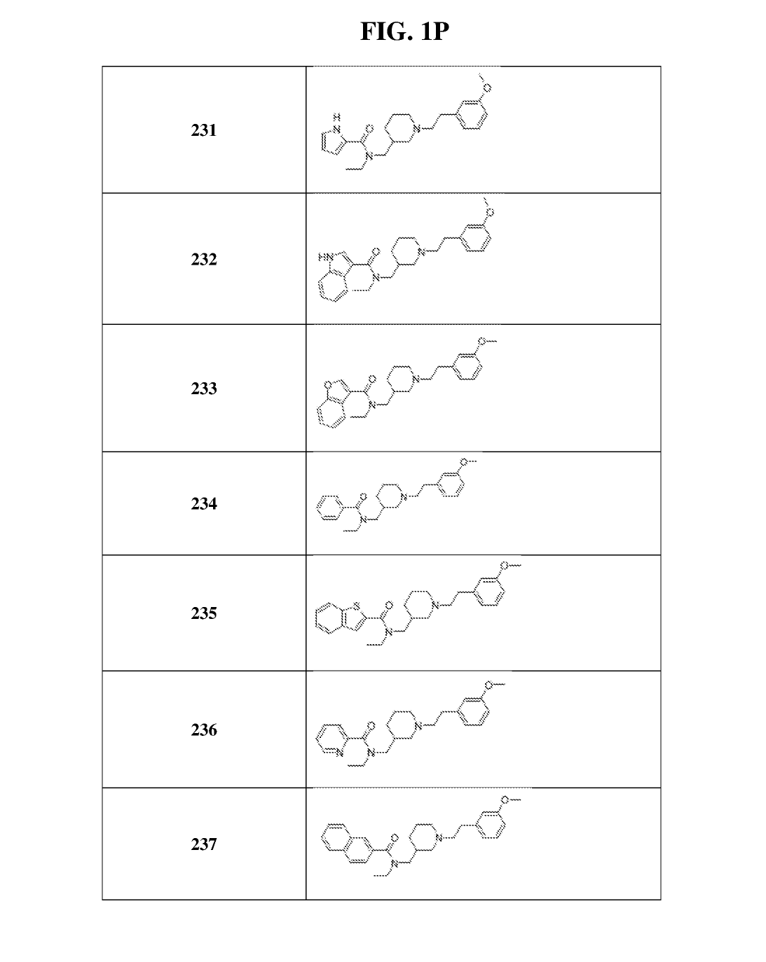

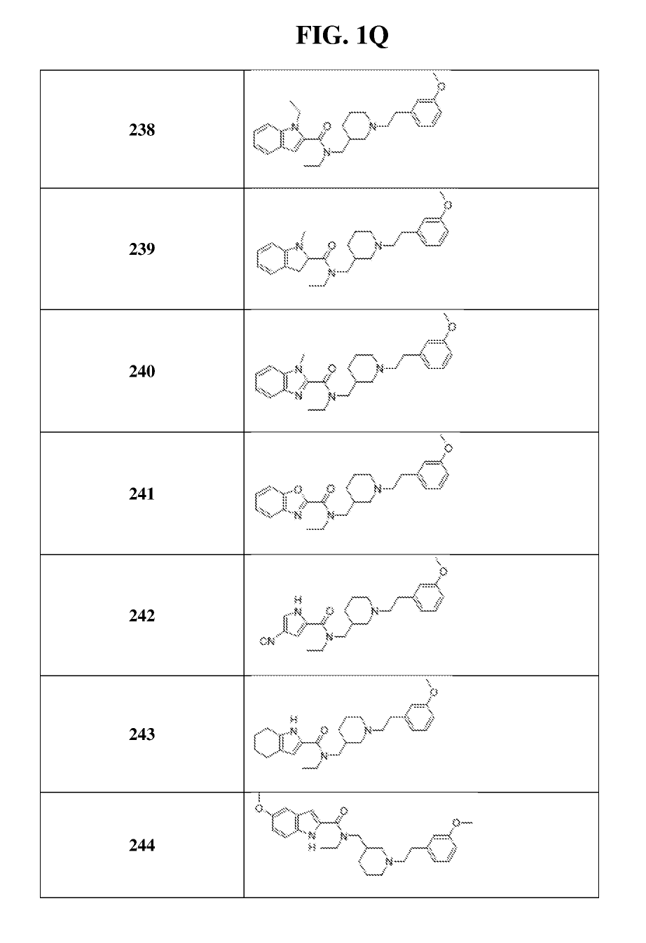

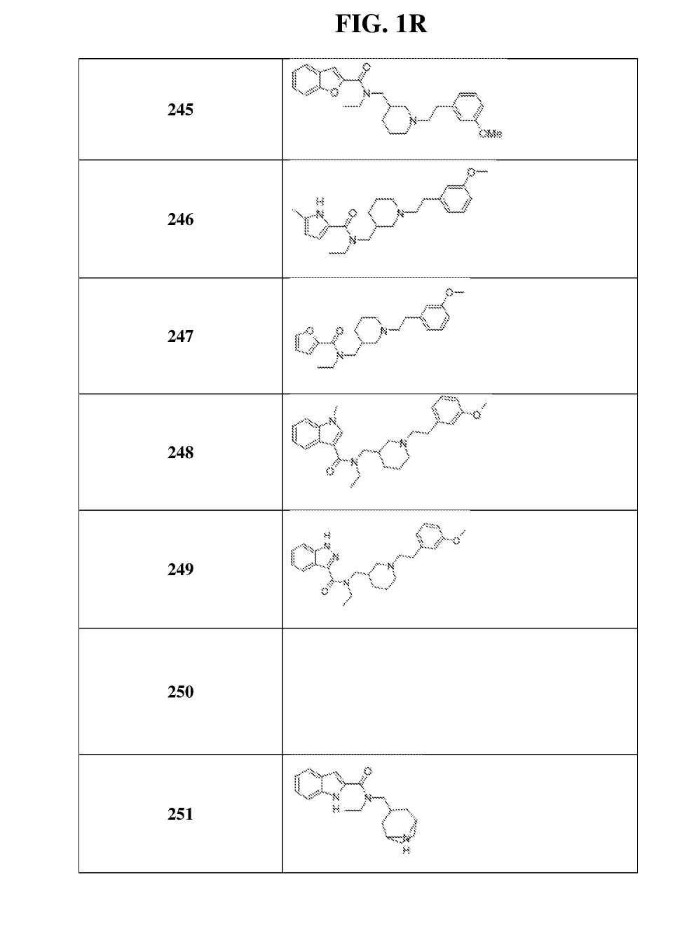

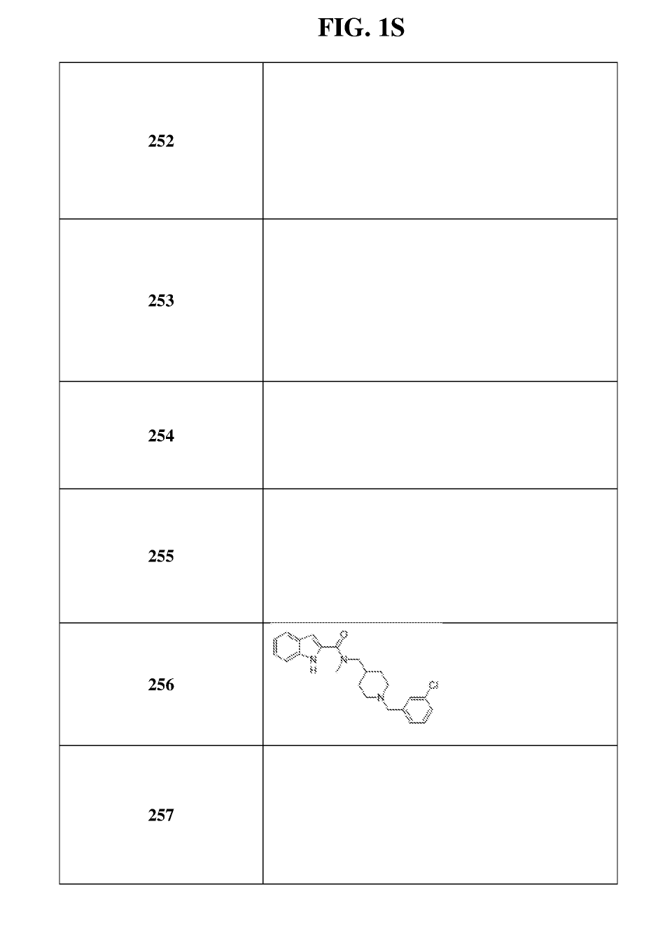

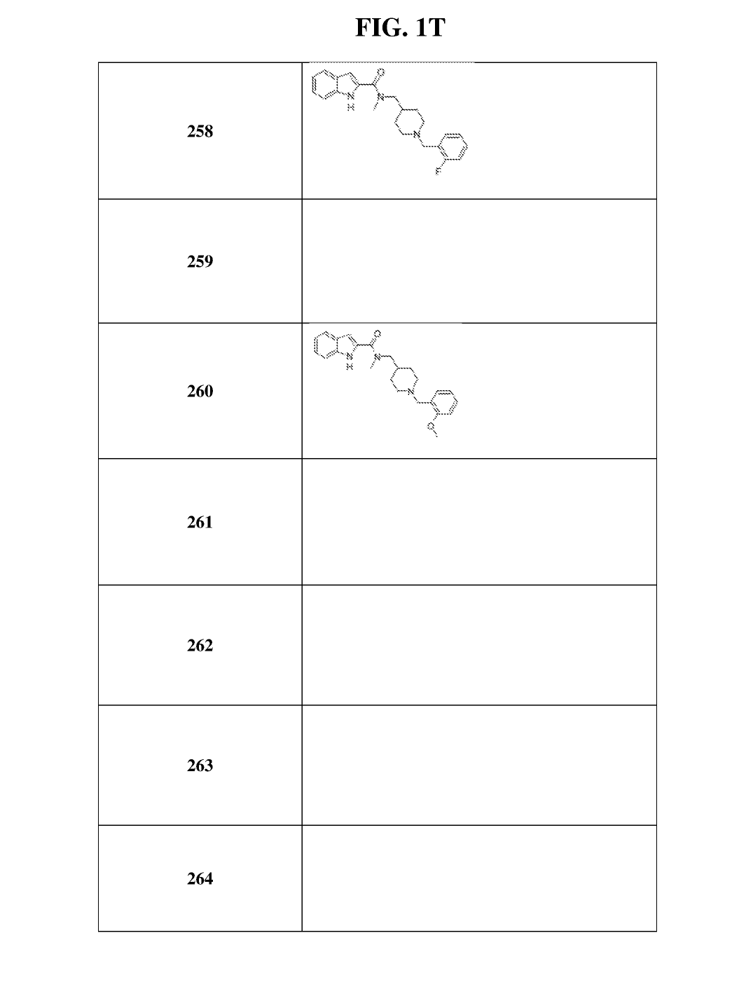

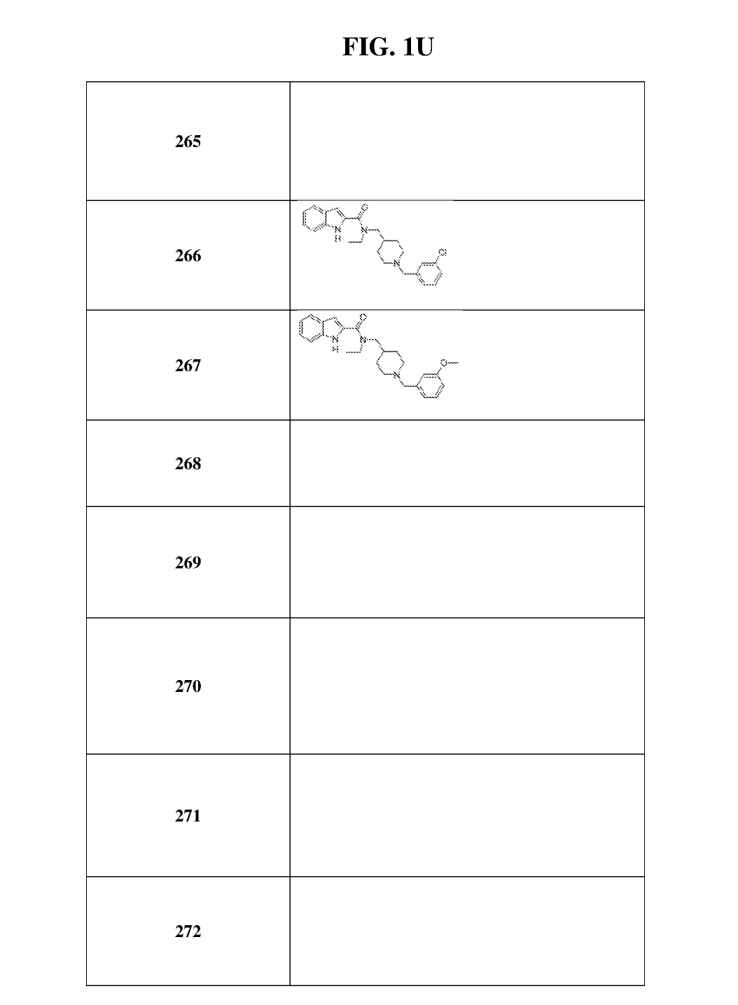

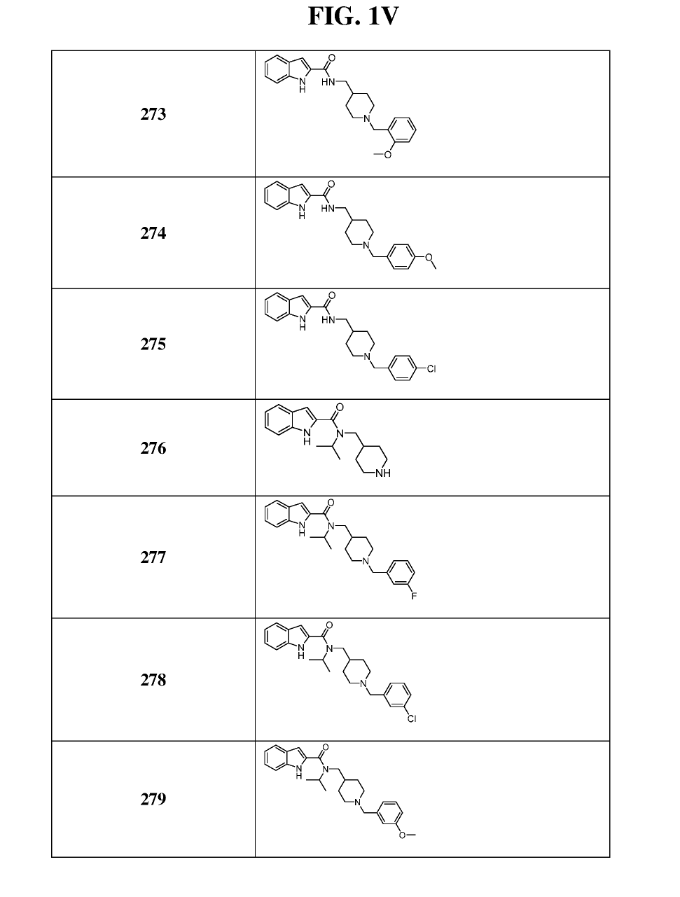

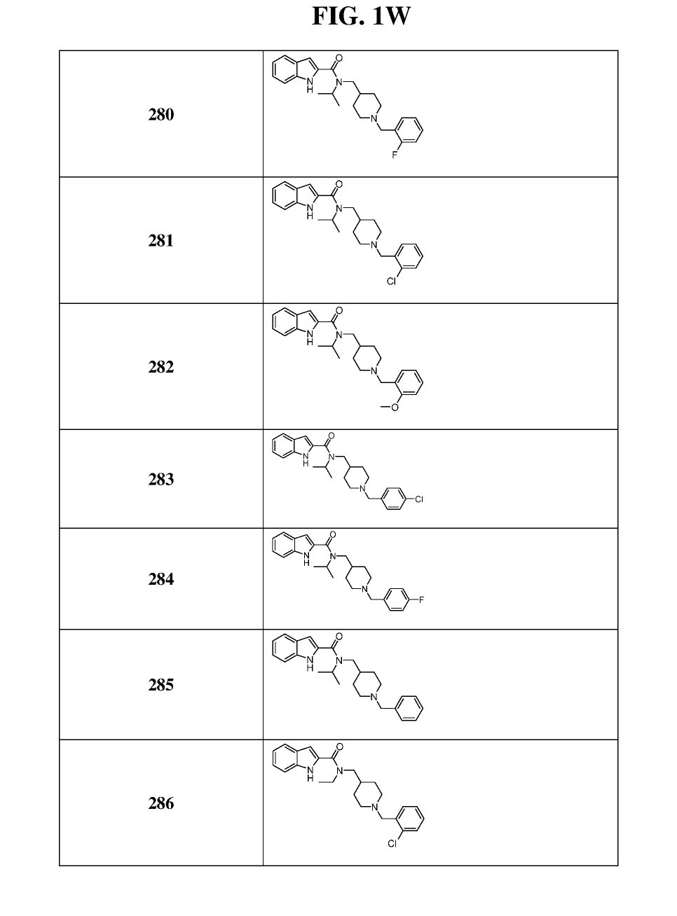

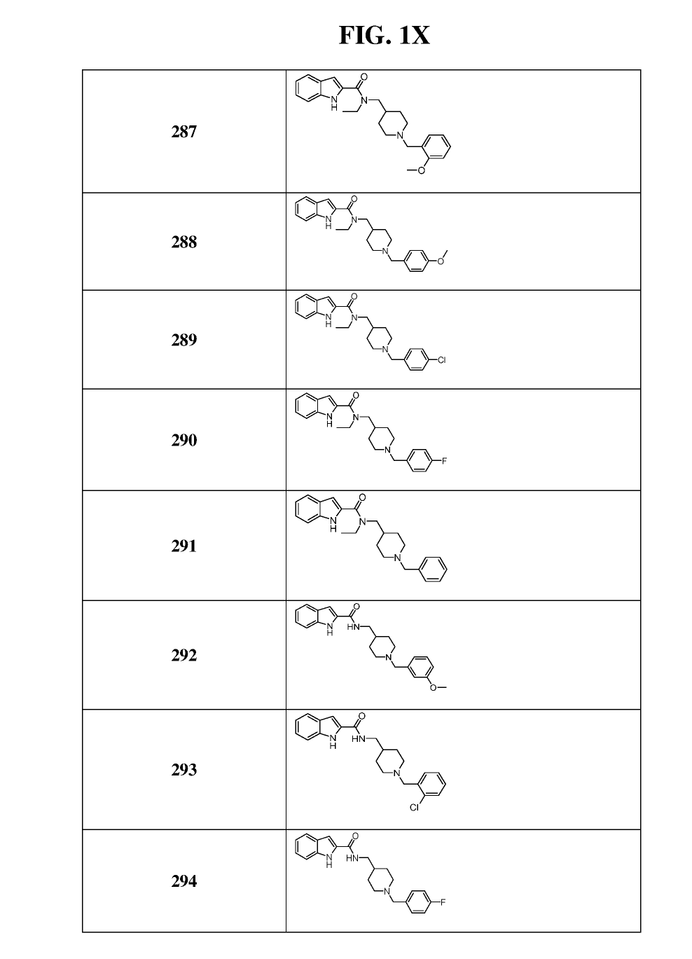

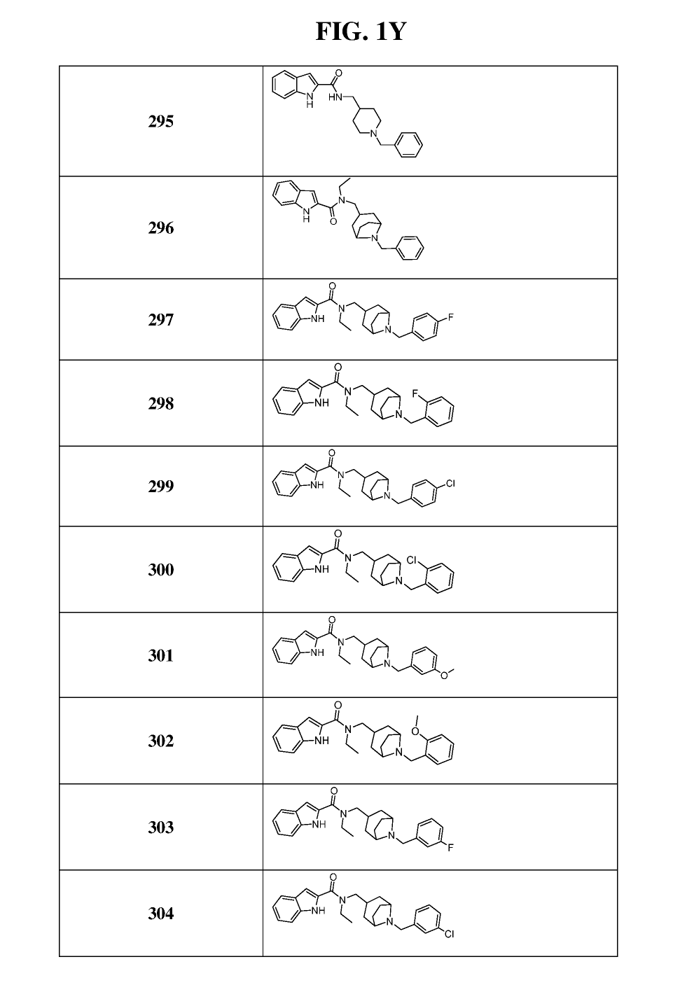

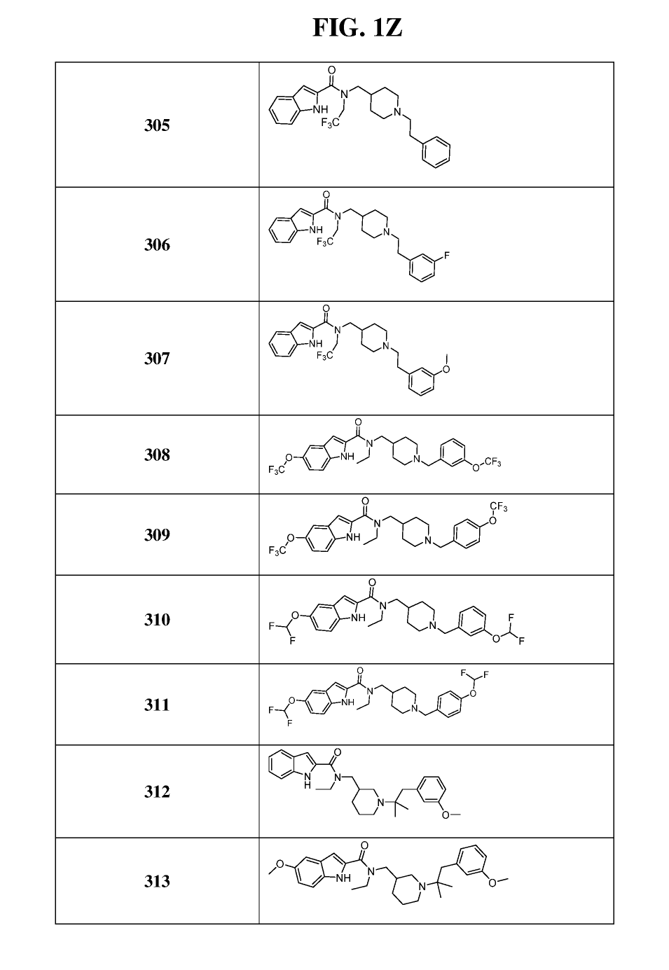

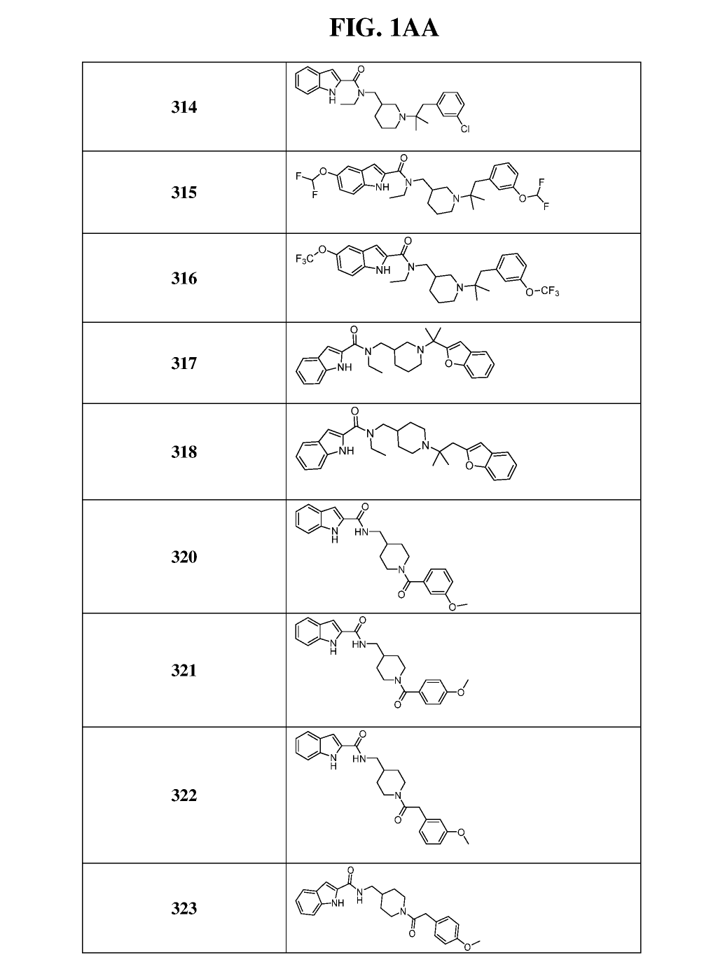

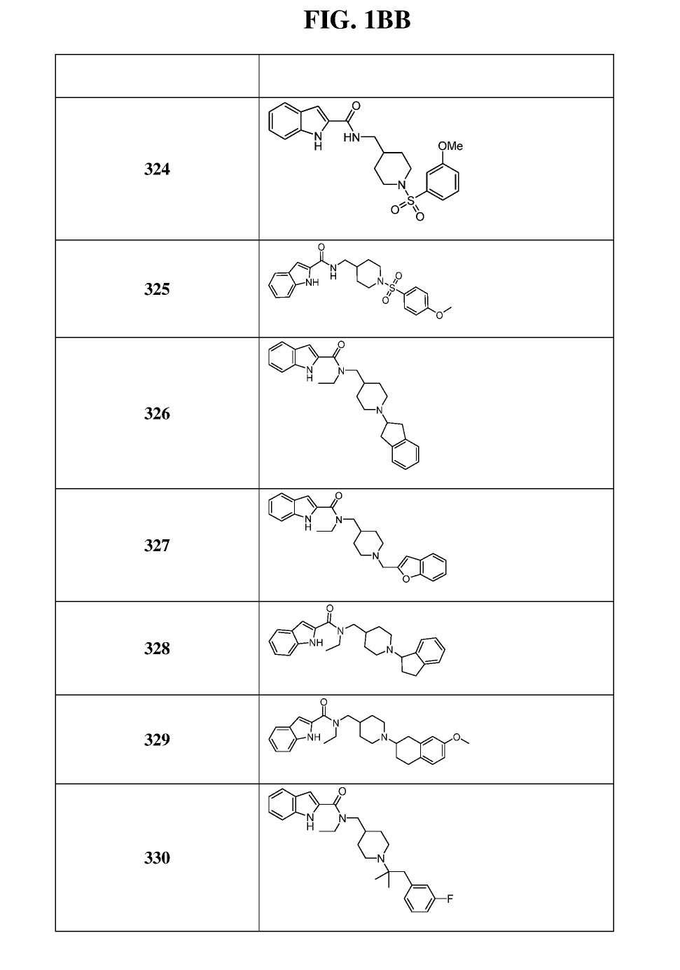

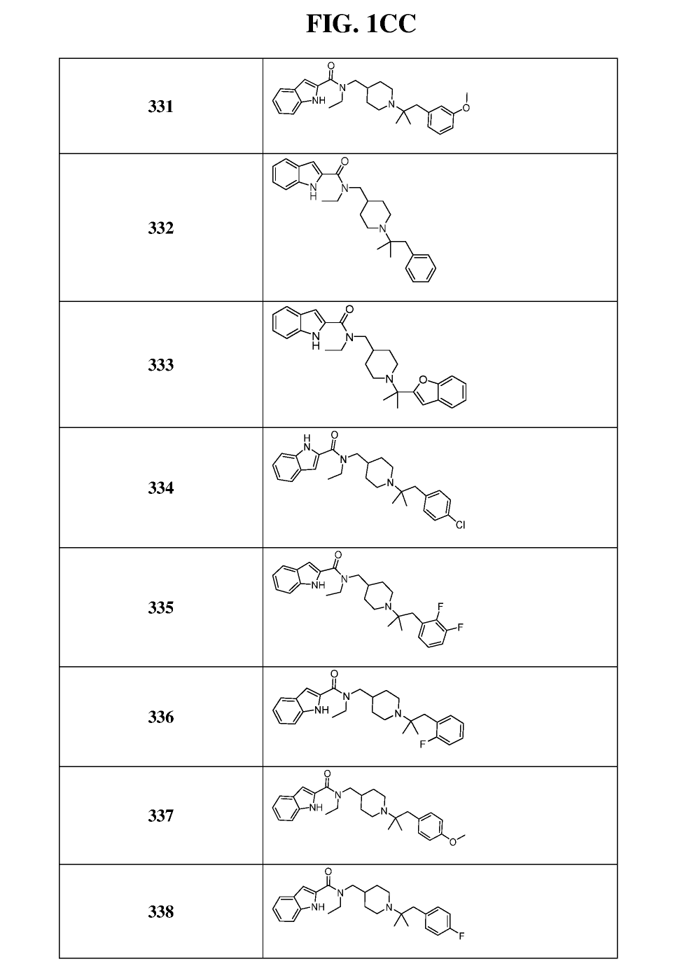

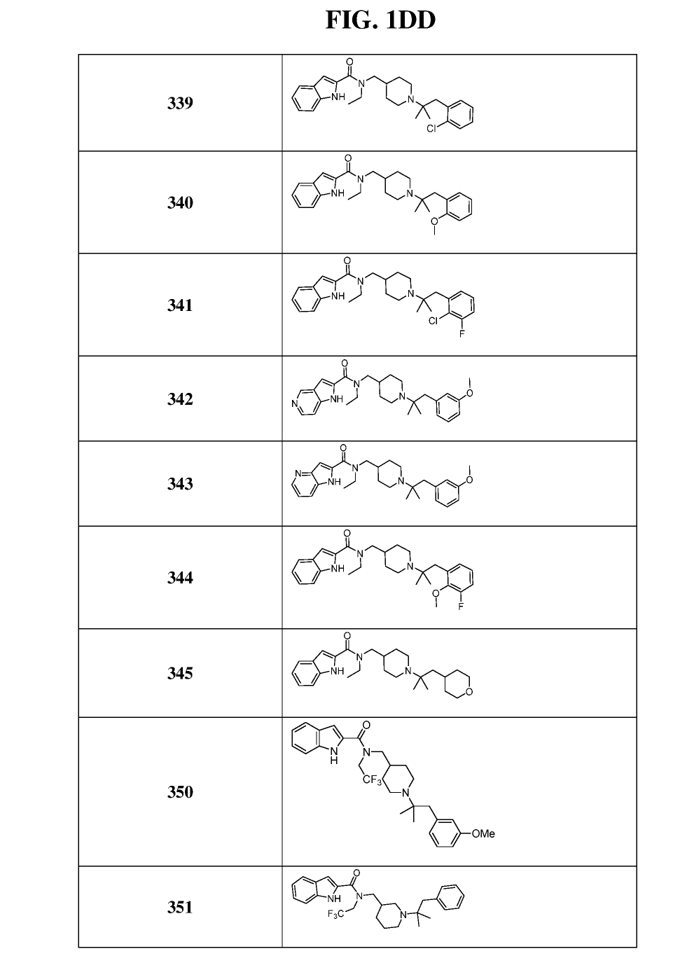

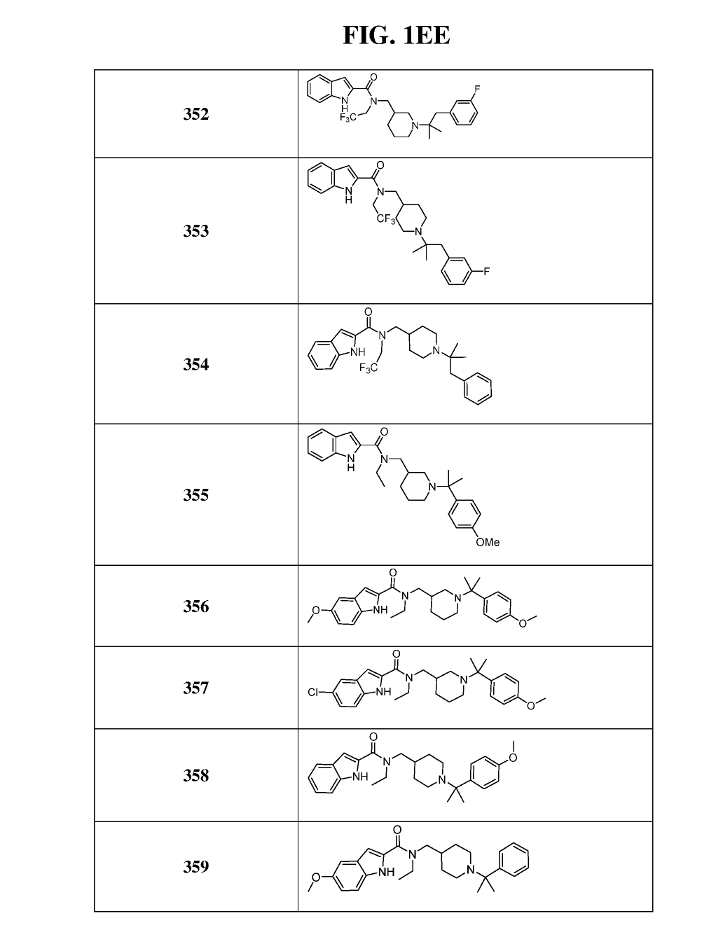

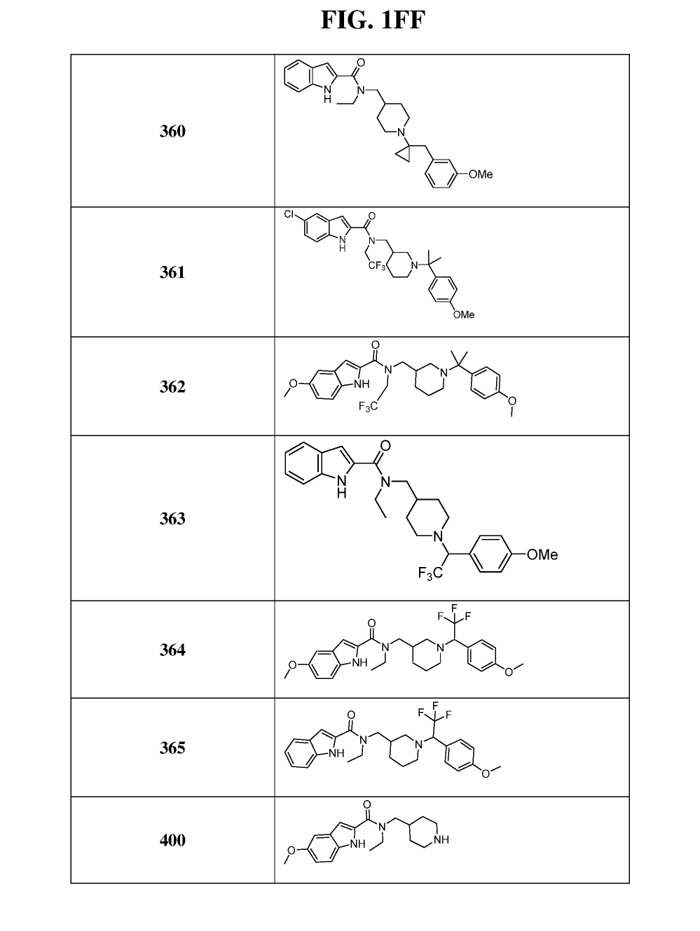

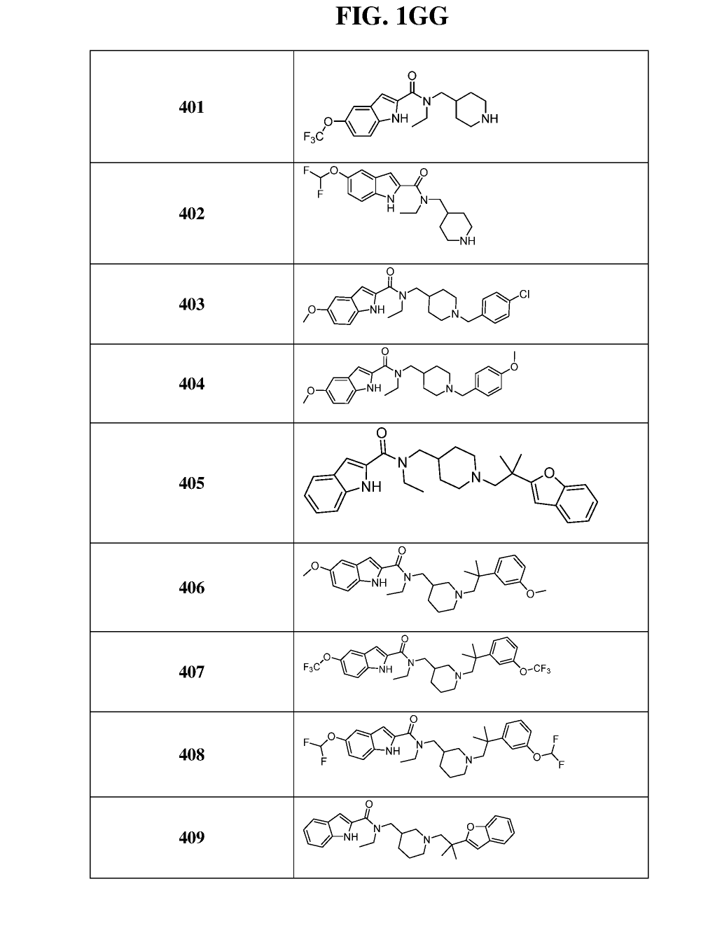

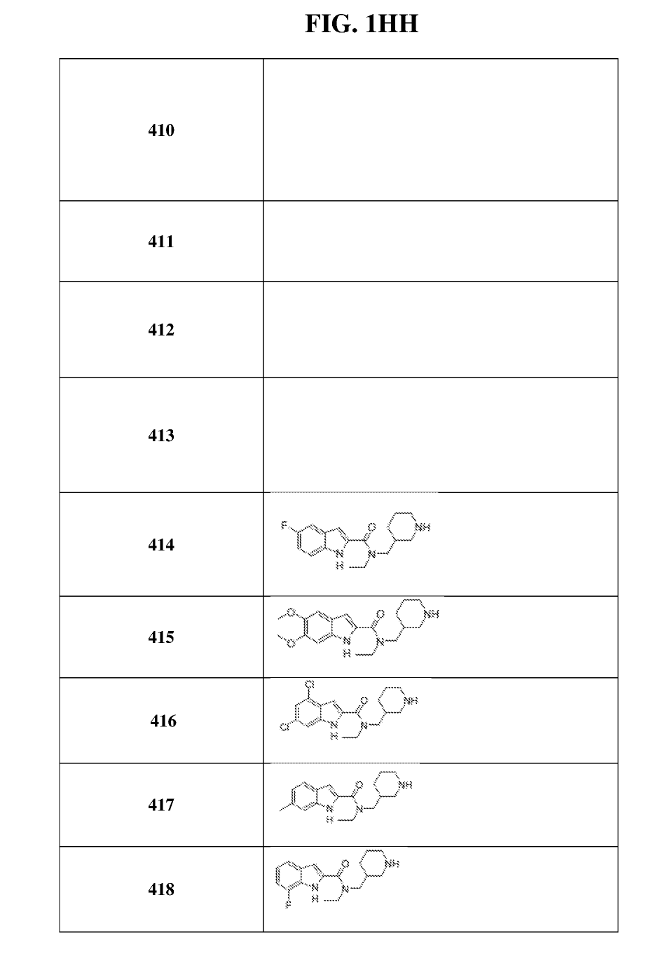

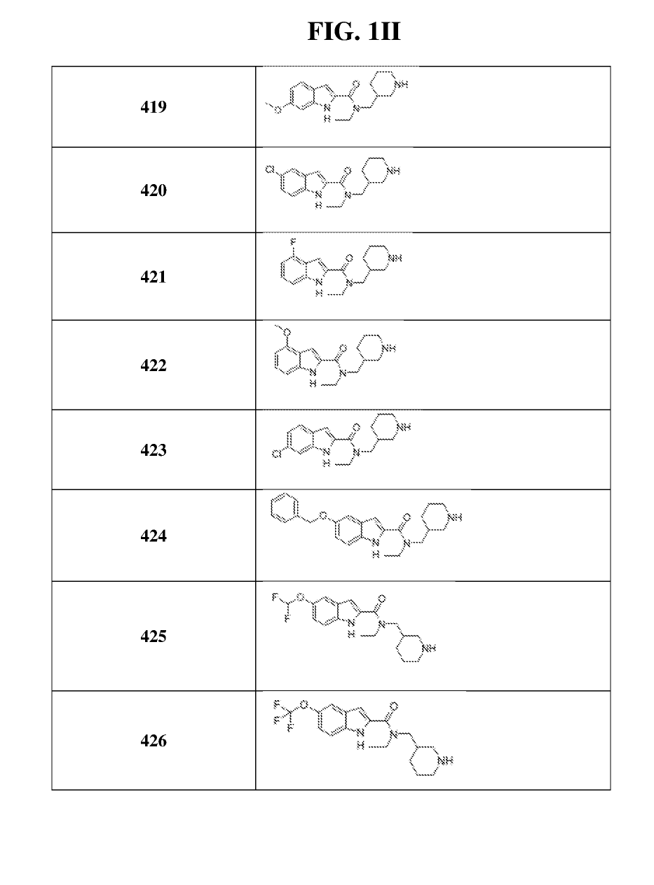

In some embodiments, the compound of Formula (I) (e.g., a compound of Formula (I-a), Formula (I-b), Formula (I-c), Formula (I-d), Formula (I-e), Formula (I-f), Formula (I-g), Formula (I-h), Formula (I-i), or Formula (I-j)) is selected from a compound depicted in FIG. 1.

In another aspect, the present invention features a compound of Formula (II):

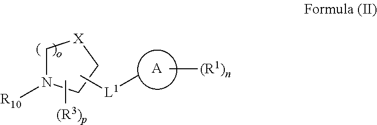

or a pharmaceutically acceptable salt thereof, wherein:

Ring A is cycloalkyl, heterocyclyl, aryl, heteroaryl;

X is C(R′), C(R′)(R″), N, or NR^{A};

L¹ is a bond, —C₁-C₆ alkyl-, —C₂-C₆ alkenyl-, —C₂-C₆ alkynyl-, —C₁-C₆ heteroalkyl-, —C(O)—, —OC(O)—, —C(O)O—, —OC(O)O—, —C(O)NR^{A}—, —NR^{A}C(O)—, —C(O)NR^{A}—C₁-C₆ alkyl, —C₁-C₆ alkyl-C(O)NR^{A}—, —NR^{A}C(O)—C₁-C₆ alkyl-, —C₁-C₆ alkyl-NR^{A}C(O)—, —C(O)NR^{A}—C₁-C₆ heteroalkyl-, —C₁-C₆ heteroalkyl-C(O)NR^{A}—, —NR^{A}C(O)—C₁-C₆ heteroalkyl-, —C₁-C₆ heteroalkyl-NR^{A}C(O)—, —C₁-C₆ alkyl-C(O)—, —C(O)—C₁-C₆ alkyl, —C₁-C₆ heteroalkyl-C(O)—, —C(O)—C₁-C₆ heteroalkyl, —C(O)—C₁-C₆ alkyl-C(O)NR^{A}—, —S(O)ₓ—, —OS(O)ₓ, —C(O)NR^{A}S(O)ₓ—, —NR^{A}S(O)ₓ—, or —S(O)ₓNR^{A}—, each of which is optionally substituted with 1-5 R⁵;

each R¹ is independently C₁-C₆ alkyl, C₂-C₆ alkenyl, C₂-C₆ alkynyl, C₁-C₆ heteroalkyl, C₁-C₆ haloalkyl, halo, cyano, cycloalkyl, heterocyclyl, aryl, heteroaryl, —OR^{B}, —NR^{A}R^{C} NR^{A}C(O)R^{D}, —S(O)ₓR^{E}, —OS(O)ₓR^{E}, —C(O)NR^{A}S(O)ₓR^{E}, —NR^{A}S(O)ₓR^{E}, or —S(O)ₓNR^{A}, each of which is optionally substituted with 1-5 R⁶;

each R³ is independently H, C₁-C₆ alkyl, C₂-C₆ alkenyl, C₂-C₆ alkynyl, C₁-C₆ heteroalkyl, C₁-C₆ haloalkyl, halo, cyano, nitro, cycloalkyl, heterocyclyl, aryl, heteroaryl, —OR^{B}, —NR^{A}R^{C}, —C(O)R^{D}, —C(O)OR^{B}, —C(O)NR^{A}R^{C}, —NR^{A}C(O)R^{D}, —NR^{A}C(O)NR^{B}R^{C}, —SR^{E}, —S(O)ₓR^{E}, —NR^{A}S(O)ₓR^{E}, or —S(O)ₓNR^{A}R^{C}, each of which is optionally substituted with 1-5 R⁷; or

or two R³, taken together with the atoms to which they are attached, form a ring (e.g., a 5-7 membered ring), optionally substituted with 1-5 R⁷;

each of R′ and R″ is independently H, C₁-C₆ alkyl, C₁-C₆ heteroalkyl, C₁-C₆ haloalkyl, halo, cyano, cycloalkyl, or heterocyclyl, each of which is optionally substituted with 1-5 R⁷;

each of R⁵, R⁶, and R⁷ is independently C₁-C₆ alkyl, C₂-C₆ alkenyl, C₂-C₆ alkynyl, C₁-C₆ heteroalkyl, C₁-C₆ haloalkyl, halo, cyano, cycloalkyl, heterocyclyl, aryl, heteroaryl, —OR^{B}, —C(O)R^{D}, —C(O)OR^{B}, —C(O)NR^{A}R^{C}, or —SR^{E}, each of which is optionally substituted with 1-5 R⁸;

each R¹⁰ is independently H, C₁-C₆ alkyl, C₂-C₆ alkenyl, C₂-C₆ alkynyl, C₁-C₆ heteroalkyl, C₁-C₆ haloalkyl, cycloalkyl, heterocyclyl, or —C(O)R^{D}, each of which is optionally substituted with 1-5 R⁸;

each R^{A}, R^{B}, R^{C}, R^{D}, or R^{E} is independently H, C₁-C₆ alkyl, C₁-C₆ heteroalkyl, C₁-C₆ haloalkyl, cycloalkyl, heterocyclyl, aryl, heteroaryl, arylalkyl, heteroarylalkyl, cycloalkylalkyl, or heterocycloalkyl, each of which is optionally substituted with 1-4 R⁸;

or R^{A} and R^{C}, together with the atoms to which each is attached, form a heterocyclyl ring optionally substituted with 1-4 R⁸;

each R⁸ is independently C₁-C₆ alkyl, C₂-C₆ alkenyl, C₂-C₆ alkynyl, C₁-C₆ heteroalkyl, C₁-C₆ haloalkyl, cycloalkyl, heterocyclyl, aryl, heteroaryl, halo, cyano, or nitro, each of which is optionally substituted with 1-5 R⁹;

each R⁹ is C₁-C₆ alkyl, halo, hydroxy, cycloalkyl, alkoxy, keto, cyano, or nitro;

n is 0, 1, 2, 3, 4, 5, or 6;

o is 1, 2, or 3;

p is 0, 1, 2, 3 or 4; and

x is 0, 1, or 2;

wherein when L¹ is connected to X, X is C(R′) or N.

In some embodiments, Ring A is aryl (e.g., monocyclic or bicyclic aryl). In some embodiments, Ring A is phenyl

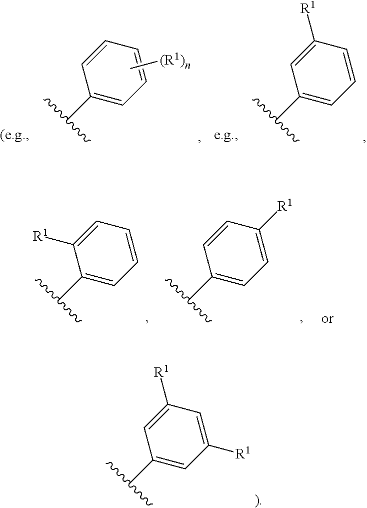

In some embodiments, Ring A is naphthyl

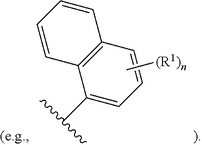

In some embodiments, R¹ is C₁-C₆ alkyl (e.g., methyl or ethyl), halo (e.g., fluoro or chloro), cyano, or —OR^{B} (e.g., —OCH₃, OCF₃, OCHF₂). In some embodiments, R¹ is —OR^{B}, (e.g., —OCH₃, OCF₃, OCHF₂). In some embodiments, n is 1 or 2.

In some embodiments, Ring A is heteroaryl. In some embodiments, Ring A is a bicyclic heteroaryl (e.g., a bicyclic nitrogen-containing heteroaryl, a bicyclic sulfur-containing heteroaryl, or a bicyclic oxygen-containing heteroaryl). In some embodiments, Ring A is indolyl, indolinyl, indazolyl, benzofuranyl, benzoimidazolyl, benzooxazolyl, or benzothiazolyl (e.g.,

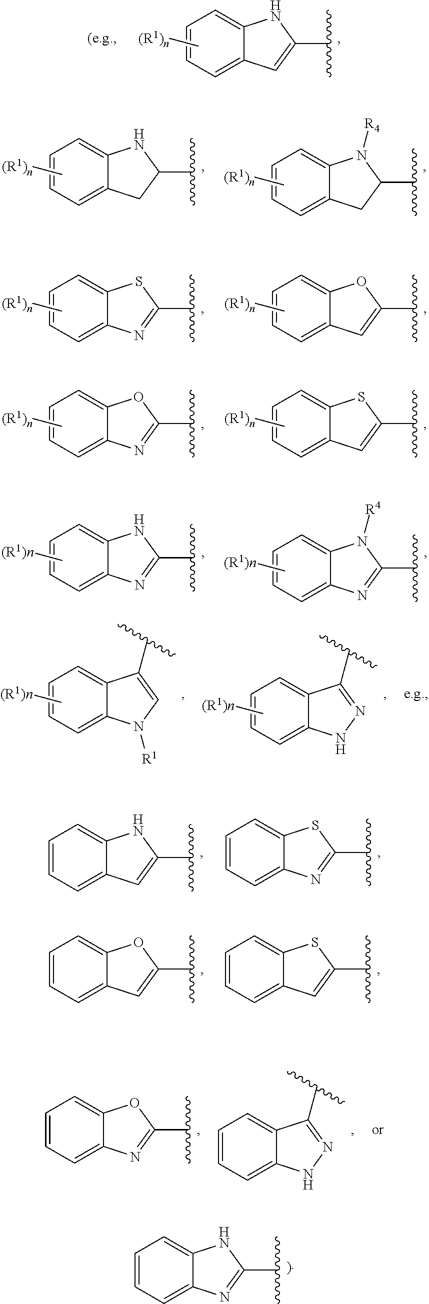

In some embodiments, n is 0.

In some embodiments, n is 1, 2, or 3. In some embodiments, n is 1 or 2. In some embodiments, n is 1.

In some embodiments, R¹ is C₁-C₆ alkyl (e.g., methyl or ethyl), halo (e.g., fluoro or chloro), cyano, or —OR^{B} (e.g., —OCH₃, OCF₃, OCHF₂, —OCH₂-aryl). In some embodiments, R¹ is —OR^{B}, (e.g., —OCH₃, OCF₃, OCHF₂).

In some embodiments, Ring A is a monocyclic heteroaryl (e.g., a monocyclic nitrogen-containing heteroaryl or monocyclic oxygen-containing heteroaryl). In some embodiments, Ring A is a 5-membered heteroaryl or a 6-membered heteroaryl. In some embodiments, Ring A is pyrrolyl, furanyl, or pyridyl,

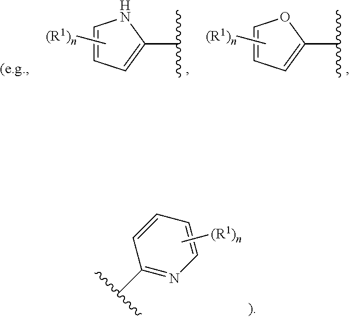

In some embodiments, X is C(R′)(R″). In some embodiments, each of R′ and R″ is independently H, C₁-C₆ alkyl, or halo. In some embodiments, each of R′ and R″ is independently H.

In some embodiments, when L¹ is connected to X, X is C(R′). In some embodiments, R′ is H. In some embodiments, when L¹ is connected to X, X is N.

In some embodiments, X is NR^{A}. In some embodiments, R^{A} is H, C₁-C₆ alkyl (methyl, ethyl, isopropyl), or C₁-C₆ heteroalkyl.

In some embodiments, L¹ is a bond, C₁-C₆ alkyl, C₁-C₆ heteroalkyl, —C(O)—, —C(O)NR^{A}—, —NR^{A}C(O)—, —C(O)NR^{A}—C₁-C₆ alkyl, —NR^{A}C(O)—C₁-C₆ alkyl, —NR^{A}C(O)—C₁-C₆ heteroalkyl, —C(O)—C₁-C₆ alkyl, C₁-C₆ alkyl-C(O)—, C₁-C₆ alkyl-NR^{A}C(O)—, —S(O)ₓ—, —OS(O)ₓ, —C(O)NR^{A}S(O)ₓ—, —NR^{A}S(O)ₓ—, or —S(O)ₓNR^{A}—, each of which is optionally substituted with 1-5 R⁵. In some embodiments, L¹ is independently a bond, C₁-C₆ alkyl, —C(O)—, —C(O)NR^{A}—C₁-C₆ alkyl, —C(O)—C₁-C₆ alkyl, or —S(O)ₓ—, each of which is optionally substituted with 1-5 R⁵.

In some embodiments, L¹ is C₁-C₆ alkyl or C₁-C₆ alkyl-NR^{A}C(O)—. In some embodiments, L¹ is C₁-C₆ alkyl-NR^{A}C(O)— (e.g., CH₂—NR^{A}C(O)—). In some embodiments, L¹ is —CH₂—N(CH₂CH₃)R^{A}C(O)—. In some embodiments, R^{A} is H, C₁-C₆ alkyl (e.g., methyl, ethyl, isopropyl), C₁-C₆ heteroalkyl, C₁-C₆ haloalkyl (e.g., CH₂CF₃), cycloalkyl (e.g., cyclohexyl), aryl (e.g., phenyl), cycloalkylalkyl, or arylalkyl (e.g., CH₂-phenyl). In some embodiments, R^{A} is H.

In some embodiments, R⁵ is C₁-C₆ alkyl (e.g., methyl or ethyl), C₁-C₆ haloalkyl (e.g., CF₃), cycloalkyl (e.g., cyclopropyl), or halo (e.g., fluoro or chloro).

In some embodiments, p is 0, 1, or 2. In some embodiments, p is 0.

In some embodiments, p is 1 or 2. In some embodiments, p is 2, and each R³ is C₁-C₆ alkyl (e.g., methyl or ethyl). In some embodiments, p is 2, and each R³ is C₁-C₆ alkyl (e.g., methyl or ethyl), wherein both R³ is joined together to form a 6- or 7-membered ring.

In some embodiments, o is 1 or 2. In some embodiments, o is 1. In some embodiments, o is 2.

In some embodiments, the compound of Formula (II) is a compound of Formula (II-a), Formula (II-b), or Formula (II-c):

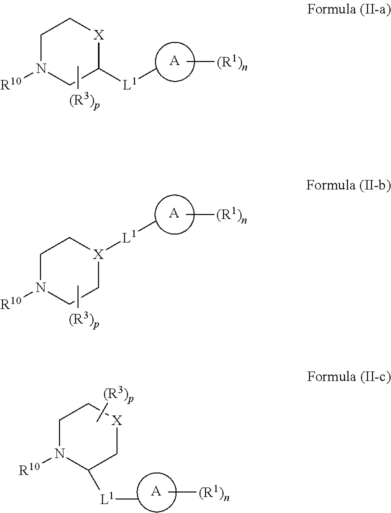

or a pharmaceutically acceptable salt thereof, wherein Ring A, L¹, R¹, R³, R¹⁰, n, p, and subvariables thereof are as described for Formula (II).

In some embodiments, the compound of Formula (II) is a compound of Formula (II-d), Formula (II-e), or Formula (II-f):

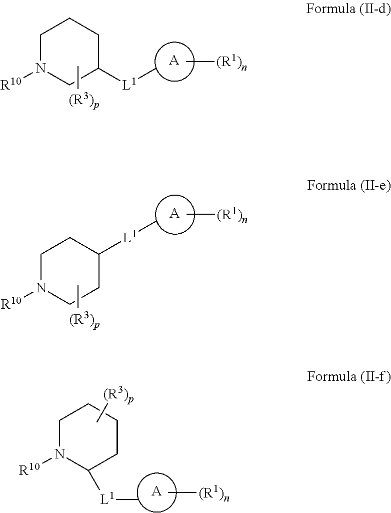

or a pharmaceutically acceptable salt thereof, wherein Ring A, L¹, R¹, R³, R¹⁰, n, p, and subvariables thereof are as described for Formula (II).

In some embodiments, the compound of Formula (II) is a compound of Formula (II-g), Formula (II-h), or Formula (II-i):

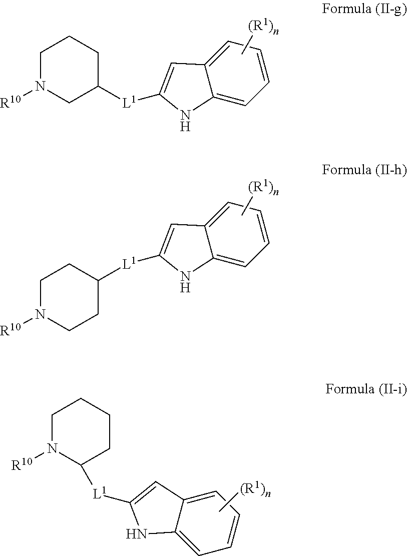

or a pharmaceutically acceptable salt thereof, wherein L¹, R¹, R¹⁰, n, and subvariables thereof are as described for Formula (II).

In some embodiments, the compound of Formula (II) (e.g., a compound of Formula (II-a), Formula (II-b), Formula (II-c), Formula (II-d), Formula (II-e), Formula (II-f), Formula (II-g), Formula (II-h), or Formula (II-i)) is selected from a compound depicted in FIG. 1.

In another aspect, the invention provides a pharmaceutical composition comprising a compound of Formula (I) or Formula (II) or a pharmaceutically acceptable salt thereof in a mixture with a pharmaceutically acceptable excipient, diluent or carrier.

In another aspect, the invention provides a method of modulating stress granule formation, the method comprising contacting a cell with a compound of Formula (I) or Formula (II). In some embodiments, stress granule formation is inhibited. In some embodiments, the stress granule is disaggregated. In some embodiments, stress granule formation is stimulated.

In some embodiments, a compound of Formula (I) or Formula (II) inhibits the formation of a stress granule. The compound of Formula (I) or Formula (II) can inhibit the formation of a stress granule by at least 10%, at least 20%, at least 30%, at least 40%, at least 50%, at least 60%, at least 70%, at least 80%, at least 90%, at least 95%, or 100% (i.e., complete inhibition) relative to a control.

In some embodiments, a compound of Formula (I) or Formula (II) disaggregates a stress granule. The compound of Formula (I) or Formula (II) can disperses or disaggregate a stress granule by at least 10%, at least 20%, at least 30%, at least 40%, at least 50%, at least 60%, at least 70%, at least 80%, at least 90%, at least 95%, or 100% (i.e., complete dispersal) relative to a control.

In some embodiments, the stress granule comprises tar DNA binding protein-43 (TDP-43), T-cell intracellular antigen 1 (TIA-1), TIA1 cytotoxic granule-associated RNA binding protein-like 1 (TIAR, TIAL1), GTPase activating protein binding protein 1 (G3BP-1), GTPase activating protein binding protein 2 (G3BP-2), tris tetraprolin (TTP, ZFP36), fused in sarcoma (FUS), or fragile X mental retardation protein (FMRP, FMR1).

In some embodiments, the stress granule comprises tar DNA binding protein-43 (TDP-43), T-cell intracellular antigen 1 (TIA-1), TIA1 cytotoxic granule-associated RNA binding protein-like 1 (TIAR, TIAL1), GTPase activating protein binding protein 1 (G3BP-1), GTPase activating protein binding protein 2 (G3BP-2), fused in sarcoma (FUS), or fragile X mental retardation protein (FMRP, FMR1).

In some embodiments, the stress granule comprises tar DNA binding protein-43 (TDP-43), T-cell intracellular antigen 1 (TIA-1), TIA1 cytotoxic granule-associated RNA binding protein-like 1 (TIAR, TIAL1), GTPase activating protein binding protein 1 (G3BP-1), GTPase activating protein binding protein 2 (G3BP-2), or fused in sarcoma (FUS).

In some embodiments, the stress granule comprises tar DNA binding protein-43 (TDP-43).

In some embodiments, the stress granule comprises T-cell intracellular antigen 1 (TIA-1).

In some embodiments, the stress granule comprises TIA-1 cytotoxic granule-associated RNA binding protein-like 1 (TIAR, TIAL1).

In some embodiments, the stress granule comprises GTPase activating protein binding protein 1 (G3BP-1).

In some embodiments, the stress granule comprises GTPase activating protein binding protein 2 (G3BP-2).

In some embodiments, the stress granule comprises tris tetraprolin (TTP, ZFP36).

In some embodiments, the stress granule comprises fused in sarcoma (FUS).

In some embodiments, the stress granule comprises fragile X mental retardation protein (FMRP, FMR1).

In another aspect, the invention provides a method of modulating TDP-43 inclusion formation, the method comprising contacting a cell with a compound of Formula (I) or Formula (II). In some embodiments, TDP-43 inclusion formation is inhibited. In some embodiments, the TDP-43 inclusion is disaggregated. In some embodiments, TDP-43 inclusion formation is stimulated.

In some embodiments, a compound of Formula (I) or Formula (II) inhibits the formation of a TDP-43 inclusion. The compound of Formula (I) or Formula (II) can inhibit the formation of a TDP-43 inclusion by at least 10%, at least 20%, at least 30%, at least 40%, at least 50%, at least 60%, at least 70%, at least 80%, at least 90%, at least 95%, or 100% (i.e., complete inhibition) relative to a control.

In some embodiments, a compound of Formula (I) or Formula (II) disaggregates a TDP-43 inclusion. The compound of Formula (I) or Formula (II) can disperses or disaggregate a TDP-43 inclusion by at least 10%, at least 20%, at least 30%, at least 40%, at least 50%, at least 60%, at least 70%, at least 80%, at least 90%, at least 95%, or 100% (i.e., complete dispersal) relative to a control.

In another aspect, the invention provides a method for treatment of a neurodegenerative disease or disorder, a musculoskeletal disease or disorder, a cancer, an ophthalmological disease or disorder (e.g., a retinal disease or disorder), and/or a viral infection, the method comprising administering an effective amount of a compound of Formula (I) or Formula (II) to a subject in need thereof.

In some embodiments, the methods are performed in a subject suffering from a neurodegenerative disease or disorder, a musculoskeletal disease or disorder, a cancer, an ophthalmological disease or disorder (e.g., a retinal disease or disorder), and/or a viral infection.

In some embodiments, the methods are performed in a subject suffering from a neurodegenerative disease or disorder. In some embodiments, the methods are performed in a subject suffering from a musculoskeletal disease or disorder. In some embodiments, the methods are performed in a subject suffering from a cancer. In some embodiments, the methods are performed in a subject suffering from an ophthalmological disease or disorder (e.g., a retinal disease or disorder). In some embodiments, the methods are performed in a subject suffering from a viral infection or viral infections.

In some embodiments, the methods comprise administering a compound of Formula (I) or Formula (II) to a subject in need thereof. In some embodiments, the subject is a mammal. In some embodiments, the subject is a nematode. In some embodiments, the subject is human.

In some embodiments, the methods further comprise the step of diagnosing the subject with a neurodegenerative disease or disorder, a musculoskeletal disease or disorder, a cancer, an ophthalmological disease or disorder (e.g., a retinal disease or disorder), or a viral infection prior to administration of a compound of Formula (I) or Formula (II). In some embodiments, the methods further comprise the step of diagnosing the subject with a neurodegenerative disease or disorder prior to administration of a compound of Formula (I) or Formula (II).

In some embodiments, the neurodegenerative disease is selected from the group consisting of Alzheimer's disease, frontotemporal dementia (FTD), FTLD-U, FTD caused by mutations in the progranulin protein or tau protein (e.g., progranulin-deficient FTLD), frontotemporal dementia with inclusion body myopathy (IBMPFD), frontotemporal dementia with motor neuron disease, amyotrophic lateral sclerosis (ALS), Huntington's disease (HD), Huntington's chorea, prion diseases (e.g., Creutzfeld-Jacob disease, bovine spongiform encephalopathy, Kuru, and scrapie), Lewy Body disease, diffuse Lewy body disease (DLBD), polyglutamine (polyQ)-repeat diseases, trinucleotide repeat diseases, cerebral degenerative diseases, presenile dementia, senile dementia, Parkinsonism linked to chromosome 17 (FTDP-17), progressive supranuclear palsy (PSP), progressive bulbar palsy (PBP), psuedobulbar palsy, spinal and bulbar muscular atrophy (SBMA), primary lateral sclerosis, Pick's disease, primary progressive aphasia, corticobasal dementia, HIV-associated dementia, Parkinson's disease, Parkinson's disease with dementia, dementia with Lewy bodies, Down's syndrome, multiple system atrophy, spinal muscular atrophy (SMA, e.g., SMA Type I (e.g., Werdnig-Hoffmann disease), SMA Type II, SMA Type III (e.g., Kugelberg-Welander disease), and congenital SMA with arthrogryposis), progressive spinobulbar muscular atrophy (e.g., Kennedy disease), post-polio syndrome (PPS), spinocerebellar ataxia, pantothenate kinase-associated neurodegeneration (PANK), spinal degenerative disease/motor neuron degenerative diseases, upper motor neuron disorder, lower motor neuron disorder, age-related disorders and dementias, Hallervorden-Spatz syndrome, cerebral infarction, cerebral trauma, chronic traumatic encephalopathy, transient ischemic attack, Lytigo-bodig (amyotrophic lateral sclerosis-parkinsonism dementia), Guam-Parkinsonism dementia, hippocampal sclerosis, corticobasal degeneration, Alexander disease, Apler's disease, Krabbe's disease, neuroborreliosis, neurosyphilis, Sandhoff disease, Tay-Sachs disease, Schilder's disease, Batten disease, Cockayne syndrome, Kearns-Sayre syndrome, Gerstmann-Straussler-Scheinker syndrome and other transmissible spongiform encephalopathies, hereditary spastic paraparesis, Leigh's syndrome, demyelinating diseases, neuronal ceroid lipofuscinoses, epilepsy, tremors, depression, mania, anxiety and anxiety disorders, sleep disorders (e.g., narcolepsy, fatal familial insomnia), acute brain injuries (e.g., stroke, head injury) autism, other diseases or disorders relating to the aberrant expression of TDP-43 and altered proteostasis, and any combination thereof.

In some embodiments, the neurodegenerative disease is selected from the group consisting of Alzheimer's disease, frontotemporal dementia (FTD), FTLD-U, FTD caused by mutations in the progranulin protein or tau protein (e.g., progranulin-deficient FTLD), amyotrophic lateral sclerosis (ALS), Huntington's disease (HD), Huntington's chorea, Creutzfeld-Jacob disease, senile dementia, Parkinsonism linked to chromosome 17 (FTDP-17), progressive supranuclear palsy (PSP), Pick's disease, primary progressive aphasia, corticobasal dementia, Parkinson's disease, Parkinson's disease with dementia, dementia with Lewy bodies, Down's syndrome, multiple system atrophy, spinal muscular atrophy (SMA), spinocerebellar ataxia, spinal degenerative disease/motor neuron degenerative diseases, Hallervorden-Spatz syndrome, cerebral infarction, cerebral trauma, chronic traumatic encephalopathy, transient ischemic attack, Lytigo-bodig (amyotrophic lateral sclerosis-parkinsonism dementia), hippocampal sclerosis, corticobasal degeneration, Alexander disease, Cockayne syndrome, and any combination thereof.

In some embodiments, the neurodegenerative disease is frontotemporal dementia (FTD). In some embodiments, the neurodegenerative disease is Alzheimer's disease or amyotrophic lateral sclerosis (ALS).

In some embodiments, the musculoskeletal disease is selected from the group consisting of muscular dystrophy, facioscapulohumeral muscular dystrophy (e.g., FSHD1 or FSHD2), Freidrich's ataxia, progressive muscular atrophy (PMA), mitochondrial encephalomyopathy (MELAS), multiple sclerosis, inclusion body myopathy, inclusion body myositis (e.g., sporadic inclusion body myositis), post-polio muscular atrophy (PPMA), motor neuron disease, myotonia, myotonic dystrophy, sacropenia, multifocal motor neuropathy, inflammatory myopathies, paralysis, and other diseases or disorders relating to the aberrant expression of TDP-43 and altered proteostasis.

In some embodiments, compounds of Formula (I) or Formula (II) may be used to prevent or treat symptoms caused by or relating to said musculoskeletal diseases, e.g., kyphosis, hypotonia, foot drop, motor dysfunctions, muscle weakness, muscle atrophy, neuron loss, muscle cramps, altered or aberrant gait, dystonias, astrocytosis (e.g., astrocytosis in the spinal cords), liver disease, respiratory disease or respiratory failure, inflammation, headache, and pain (e.g., back pain, neck pain, leg pain, or inflammatory pain).

In some embodiments, the cancer is selected from the group consisting of breast cancer, a melanoma, adrenal gland cancer, biliary tract cancer, bladder cancer, brain or central nervous system cancer, bronchus cancer, blastoma, carcinoma, a chondrosarcoma, cancer of the oral cavity or pharynx, cervical cancer, colon cancer, colorectal cancer, esophageal cancer, gastrointestinal cancer, glioblastoma, hepatic carcinoma, hepatoma, kidney cancer, leukemia, liver cancer, lung cancer, lymphoma, non-small cell lung cancer, ophthalmological cancer, osteosarcoma, ovarian cancer, pancreas cancer, peripheral nervous system cancer, prostate cancer, sarcoma, salivary gland cancer, small bowel or appendix cancer, small-cell lung cancer, squamous cell cancer, stomach cancer, testis cancer, thyroid cancer, urinary bladder cancer, uterine or endometrial cancer, vulval cancer, and any combination thereof.

In some embodiments, the cancer is selected from the group consisting of blastoma, carcinoma, a glioblastoma, hepatic carcinoma, lymphoma, leukemia, and any combination thereof.

In some embodiments, the cancer is selected from Hodgkin's lymphoma or non-Hodgkin's lymphoma. In some embodiments, the cancer is a non-Hodgkin's lymphoma, selected from the group consisting of a B-cell lymphoma (e.g., diffuse large B-cell lymphoma, primary mediastinal B-cell lymphoma, intravascular large B-cell lymphoma, follicular lymphoma, chronic lymphocytic leukemia/small lymphocytic lymphoma, mantle cell lymphoma, marginal zone B-cell lymphomas, extranodal marginal B-cell lymphomas, mucosa-associated lymphoid tissue (MALT) lymphomas, modal marginal zone B-cell lymphoma, splenic marginal zone B-cell lymphoma, Burkitt lymphoma, lymphoplasmacytic lymphoma, Waldenström's macroglobulinemia, hairy cell leukemia, and primary central nervous system (CNS) lymphoma) and a T-cell lymphoma (e.g., precursor T-lymphoblastic lymphoma/leukemia, peripheral T-cell lymphoma, cutaneous T-cell lymphoma, adult T-cell lymphoma (e.g., smoldering adult T-cell lymphoma, chronic adult T-cell lymphoma, acute adult T-cell lymphoma, lymphomatous adult T-cell lymphoma), angioimmunoblastic T-cell lymphoma, extranodal natural killer T-cell lymphoma nasal type (ENKL), enteropathy-associated intestinal T-cell lymphoma (EATL) (e.g., Type I EATL and Type II EATL), and anaplastic large cell lymphoma (ALCL)).

In some embodiments, the ophthalmological disease or disorder (e.g., retinal disease or disorder) is selected from macular degeneration (e.g., age-related macular degeneration), diabetes retinopathy, histoplasmosis, macular hole, macular pucker, Bietti's crystalline dystrophy, retinal detachment, retinal thinning, retinoblastoma, retinopathy of prematurity, Usher's syndrome, vitreous detachment, Refsum disease, retinitis pigmentosa, onchocerciasis, choroideremia, Leber congenital amaurosis, retinoschisis (e.g., juvenile retinoschisis), Stargardt disease, ophthalmoplegia, and the like.

In some embodiments, the ophthalmological disease or disorder (e.g., retinal disease or disorder) is selected from macular degeneration (e.g., age-related macular degeneration), diabetes retinopathy, histoplasmosis, macular hole, macular pucker, Bietti's crystalline dystrophy, retinoblastoma, retinopathy of prematurity, Usher's syndrome, Refsum disease, retinitis pigmentosa, onchocerciasis, choroideremia, Leber congenital amaurosis, retinoschisis (e.g., juvenile retinoschisis), Stargardt disease, and the like.

In some embodiments, the viral infection is caused by a virus selected from the group consisting of West Nile virus, respiratory syncytial virus (RSV), herpes simplex virus 1, herpes simplex virus 2, Epstein-Barr virus (EBV), hepatitis virus A, hepatitis virus B, hepatitis virus C, influenza viruses, chicken pox, avian flu viruses, smallpox, polio viruses, HIV-1, HIV-2, Ebola virus, and any combination thereof.

In some embodiments, the viral infection is caused by a virus selected from the group consisting of herpes simplex virus 1, herpes simplex virus 2, Epstein-Barr virus (EBV), hepatitis virus A, hepatitis virus B, hepatitis virus C, HIV-1, HIV-2, Ebola virus, and any combination thereof.

In some embodiments, the viral infection is HIV-1 or HIV-2.

In some embodiments, the pathology of the neurodegenerative disease or disorder, musculoskeletal disease or disorder, cancer, ophthalmological disease or disorder (e.g., retinal disease or disorder), and/or viral infection comprises stress granules.

In some embodiments, pathology of the disease or disorder comprises stress granules. By comprising stress granules is meant that number of stress granules in a cell in the subject is changed relative to a control and/or healthy subject or relative to before onset of said disease or disorder. Exemplary diseases and disorders pathology of which incorporate stress granules include, but are not limited to, neurodegenerative diseases, musculoskeletal diseases, cancers, ophthalmological diseases (e.g., retinal diseases), and viral infections.

In another aspect, the invention provides methods of diagnosing a neurodegenerative disease, a musculoskeletal disease, a cancer, an ophthalmological disease (e.g., a retinal disease), or a viral infection in a subject, the method comprising administering a compound of Formula (I) or Formula (II) to the subject. In some embodiments, the invention provides methods of diagnosing a neurodegenerative disease in a subject, the method comprising administering a compound of Formula (I) or Formula (II) to the subject. For use in diagnosis, a compound of Formula (I) or Formula (II) can be modified with a label.

In another aspect, the invention provides methods of modulating stress granules comprising contacting a cell with a compound of Formula (I) or Formula (II).

In another aspect, the invention provides methods of modulating TDP-43 inclusion formation comprising contacting a cell with a compound of Formula (I) or Formula (II). In some embodiments, TDP-43 is inducibly expressed. In some embodiments, the cell line is a neuronal cell line.

In some embodiments, the cell is treated with a physiochemical stressor. In some embodiments, the physicochemical stressor is selected from arsenite, nutrient deprivation, heat shock, osmotic shock, a virus, genotoxic stress, radiation, oxidative stress, oxidative stress, a mitochondrial inhibitor, and an endoplasmic reticular stressor. In some embodiments, the physicochemical stressor is ultraviolet or x-ray radiation. In some embodiments, the physicochemical stressor is oxidative stress induced by FeCl₂ or CuCl₂ and a peroxide.

In yet another aspect, the invention provides a method of screening for modulators of TDP-43 aggregation comprising contacting a compound of Formula (I) or Formula (II) with a cell that expresses TDP-43 and develops spontaneous inclusions.

In some embodiments, the stress granule comprises TDP-43, i.e., is a TDP-43 inclusion. Accordingly, in some embodiments, a compound of Formula (I) or Formula (II) is a modulator of TDP-43 inclusions.

In another aspect, the invention provides a method of treating a B-cell or T-cell lymphoma, the method comprising administering a compound of Formula (I) to a subject in need thereof:

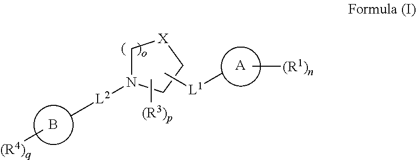

or a pharmaceutically acceptable salt thereof, wherein Ring A, Ring B, L¹, L², R¹, R³, R⁴, n, p, q, and subvariables thereof are as described for Formula (I) herein.

In some embodiments, the B-cell or T-cell lymphoma is selected from the group consisting of diffuse large B-cell lymphoma, primary mediastinal B-cell lymphoma, intravascular large B-cell lymphoma, follicular lymphoma, chronic lymphocytic leukemia/small lymphocytic lymphoma, mantle cell lymphoma, marginal zone B-cell lymphomas, extranodal marginal B-cell lymphomas, mucosa-associated lymphoid tissue (MALT) lymphomas, modal marginal zone B-cell lymphoma, splenic marginal zone B-cell lymphoma, Burkitt lymphoma, lymphoplasmacytic lymphoma, Waldenström's macroglobulinemia, hairy cell leukemia, primary central nervous system (CNS) lymphoma, precursor T-lymphoblastic lymphomalleukemia, peripheral T-cell lymphoma, smoldering adult T-cell lymphoma, chronic adult T-cell lymphoma, acute adult T-cell lymphoma, lymphomatous adult T-cell lymphoma, angioimmunoblastic T-cell lymphoma, extranodal natural killer T-cell lymphoma nasal type (ENKL), enteropathy-associated intestinal T-cell lymphoma (EATL), and anaplastic large cell lymphoma (ALCL).

In another aspect, the invention provides a method of treating a neurodegenerative disease selected from the group consisting of frontotemporal dementia caused by mutations in the progranulin protein or tau protein (e.g., progranulin-deficient FTLD), frontotemporal dementia with inclusion body myopathy (IBMPFD), frontotemporal dementia with motor neuron disease, bovine spongiform encephalopathy, Kuru, scrapie, Lewy Body disease, diffuse Lewy body disease (DLBD), polyglutamine (polyQ)-repeat diseases, progressive bulbar palsy (PBP), psuedobulbar palsy, spinal and bulbar muscular atrophy (SBMA), primary lateral sclerosis, HIV-associated dementia, progressive spinobulbar muscular atrophy (e.g., Kennedy disease), post-polio syndrome (PPS), pantothenate kinase-associated neurodegeneration (PANK), Lytigo-bodig (amyotrophic lateral sclerosis-parkinsonism dementia), Guam-Parkinsonism dementia, hippocampal sclerosis, corticobasal degeneration, Alexander disease, Apler's disease, Krabbe's disease, neuroborreliosis, neurosyphilis, Sandhoff disease, Tay-Sachs disease, Schilder's disease, Batten disease, Cockayne syndrome, Kearns-Sayre syndrome, Gerstmann-Straussler-Scheinker syndrome and other transmissible spongiform encephalopathies, hereditary spastic paraparesis, Leigh's syndrome, demyelinating diseases, neuronal ceroid lipofuscinoses, epilepsy, tremors, depression, mania, anxiety and anxiety disorders, sleep disorders (e.g., narcolepsy, fatal familial insomnia), acute brain injuries (e.g., stroke, head injury) or autism, by administering a compound of Formula (I) to a subject in need thereof:

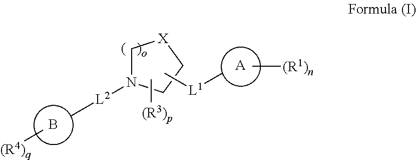

or a pharmaceutically acceptable salt thereof, wherein Ring A, Ring B, L¹, L², R¹, R³, R⁴, n, p, q, and subvariables thereof are as described for Formula (I) herein.

In another aspect, the invention provides a method of treating a musculoskeletal disease by administering a compound of Formula (I) to a subject in need thereof:

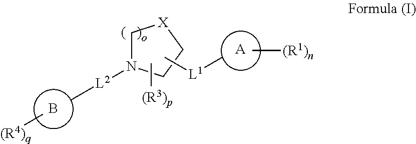

or a pharmaceutically acceptable salt thereof, wherein Ring A, Ring B, L¹, L², R¹, R³, R⁴, n, p, q, and subvariables thereof are as described for Formula (I) herein.

In some embodiments, the musculoskeletal disease is selected from the group consisting of muscular dystrophy, facioscapulohumeral muscular dystrophy (e.g., FSHD1 or FSHD2), Freidrich's ataxia, progressive muscular atrophy (PMA), mitochondrial encephalomyopathy (MELAS), multiple sclerosis, inclusion body myopathy, inclusion body myositis (e.g., sporadic inclusion body myositis), post-polio muscular atrophy (PPMA), motor neuron disease, myotonia, myotonic dystrophy, sacropenia, multifocal motor neuropathy, inflammatory myopathies, and paralysis.

In another aspect, the invention provides a method of treating an ophthalmological disease or disorder, the method comprising administering a compound of Formula (I) to a subject in need thereof:

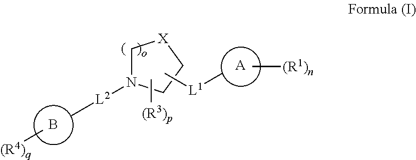

or a pharmaceutically acceptable salt thereof, wherein Ring A, Ring B, L¹, L², R¹, R³, R⁴, n, p, q, and subvariables thereof are as described for Formula (I) herein.

In some embodiments, the ophthalmological disease (e.g., retinal disease) is selected from the group consisting of macular degeneration, age-related macular degeneration, diabetes retinopathy, histoplasmosis, macular hole, macular pucker, Bietti's crystalline dystrophy, retinal detachment, retinal thinning, retinoblastoma, retinopathy of prematurity, Usher's syndrome, vitreous detachment, Refsum disease, retinitis pigmentosa, onchocerciasis, choroideremia, Leber congenital amaurosis, retinoschisis, juvenile retinoschisis, Stargardt disease, ophthalmoplegia, or any combination thereof.

In another aspect, the invention provides a method of treating a viral infection caused by the Ebola virus, the method comprising administering a compound of Formula (I) to a subject in need thereof:

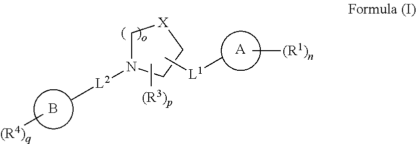

or a pharmaceutically acceptable salt thereof, wherein Ring A, Ring B, L¹, L², R¹, R³, R⁴, n, p, q, and subvariables thereof are as described for Formula (I) herein.

In any and all aspects, in some embodiments, the compound of Formula (I) is selected from a compound depicted in FIG. 1.

In some embodiments, the subject is a mammal. In some embodiments, the subject is human.

In some embodiments, the method further comprises the step of diagnosing the subject with the neurodegenerative disease or disorder, musculoskeletal disease or disorder, cancer, ophthalmological disease or disorder, or viral infection prior to onset of said administration. In some embodiments, the pathology of said neurodegenerative disease or disorder, said musculoskeletal disease or disorder, said cancer, said ophthalmological disease or disorder, and said viral infection comprises stress granules. In some embodiments, the pathology of said neurodegenerative disease, said musculoskeletal disease or disorder, said cancer, said ophthalmological disease or disorder, and said viral infection comprises TDP-43 inclusions.

TDP-43 and other RNA-binding proteins function in both the nucleus and cytoplasm to process mRNA, e.g., by splicing mRNA, cleaving mRNA introns, cleaving untranslated regions of mRNA or modifying protein translation at the synapse, axon, dendrite or soma. Therefore, targeting other proteins that function in an analogous manner to TDP-43 or by processing mRNA may also be beneficial to prevent and treat neurodegeneration resulting from disease. For instance, the fragile X mental retardation 1 (FMRP) protein is essential for normal cognitive development (Nakamoto, M., et al. (2007) Proc Natl Acad Sci U.S.A. 104:15537-15542). The signaling systems that affect TDP-43 function might also affect this protein, thus improving cognitive function. This can be particularly important at the synapse where neurons communicate. Without wishing to be bound by a theory, the signaling systems that compounds of Formula (I) target may also modify these processes, which play a role in neurodegeneration or mental health illnesses (e.g., schizophrenia).

The cellular stress response follows a U-shaped curve. Overinduction of this pathway, such as observed in many neurodegenerative diseases, can be harmful for cells. However, a decreased stimulation of this pathway can also be harmful for cells, e.g., in the case of an acute stress, such as a stroke. Thus, the appropriate action for some diseases is the inhibition of stress granule formation, while for other diseases, stimulation of stress granule formation is beneficial.

In some embodiments, the TDP-43 protein in a stress granule may be wild-type or a mutant form of TDP-43. In some embodiments, the mutant form of TDP-43 comprises an amino acid addition, deletion, or substitution, e.g., relative to the wild type sequence of TDP-43. In some embodiments, the mutant form of TDP-43 comprises an amino acid substitution relative to the wild type sequence, e.g., a G294A, A135T, Q331K, or Q343R substitution. In some embodiments, the TDP-43 protein in a stress granule comprises a post-translational modification, e.g., phosphorylation of an amino acid side chain, e.g., T103, S104, S409, or S410. In some embodiments, post-translational modification of the TDP-43 protein in a stress granule may be modulated by treatment with a compound of the invention.

### Methods of Treatment

Neurodegenerative Diseases:

Without wishing to be bound by a theory, compounds of Formula (I) can be used to delay the progression of neurodegenerative illnesses where the pathology incorporates stress granules. Such illnesses include ALS and frontotemporal dementia, in which TDP-43 is the predominant protein that accumulates to form the pathology. This group also includes Alzheimer's disease and FTLD-U, where TDP-43 and other stress granule proteins co-localize with tau pathology. Because modulators of TDP-43 inclusions, such as compounds of Formula (I), can act to block the enzymes that signal stress granule formation (e.g., the three enzymes that phosphorylate eIF2a: PERK, GCN2 and HRI), compounds of Formula (I) may also reverse stress granules that might not include TDP-43. Accordingly, compounds of Formula (I) can be used for treatment of neurodegenerative diseases and disorders in which the pathology incorporates stress granules, such as Huntington's chorea and Creutzfeld-Jacob disease. Compounds of Formula (I) may also be used for treatment of neurodegenerative diseases and disorders that involve TDP-43 multisystem proteinopathy.

The term “neurodegenerative disease” as used herein, refers to a neurological disease characterized by loss or degeneration of neurons. The term “neurodegenerative disease” includes diseases caused by the involvement of genetic factors or the cell death (apoptosis) of neurons attributed to abnormal protein accumulation and so on. Additionally, neurodegenerative diseases include neurodegenerative movement disorders and neurodegenerative conditions relating to memory loss and/or dementia. Neurodegenerative diseases include tauopathies and α-synucleopathies. Exemplary neurodegenerative diseases include, but are not limited to, Alzheimer's disease, frontotemporal dementia (FTD), FTLD-U, FTD caused by mutations in the progranulin protein or tau protein (e.g., progranulin-deficient FTLD), frontotemporal dementia with inclusion body myopathy (IBMPFD), frontotemporal dementia with motor neuron disease, amyotrophic lateral sclerosis (ALS), amyotrophic lateral sclerosis with dementia (ALSD), Huntington's disease (HD), Huntington's chorea, prion diseases (e.g., Creutzfeld-Jacob disease, bovine spongiform encephalopathy, Kuru, or scrapie), Lewy Body disease, diffuse Lewy body disease (DLBD), polyglutamine (polyQ)-repeat diseases, trinucleotide repeat diseases, cerebral degenerative diseases, presenile dementia, senile dementia, Parkinsonism linked to chromosome 17 (FTDP-17), progressive supranuclear palsy (PSP), progressive bulbar palsy (PBP), psuedobulbar palsy, spinal and bulbar muscular atrophy (SBMA), primary lateral sclerosis, Pick's disease, primary progressive aphasia, corticobasal dementia, HIV-associated dementia, Parkinson's disease, Parkinson's disease with dementia, dementia with Lewy bodies, Down's syndrome, multiple system atrophy, spinal muscular atrophy (SMA, e.g., SMA Type I (e.g., Werdnig-Hoffmann disease) SMA Type II, SMA Type III (e.g., Kugelberg-Welander disease), and congenital SMA with arthrogryposis), progressive spinobulbar muscular atrophy (e.g., Kennedy disease), post-polio syndrome (PPS), spinocerebellar ataxia, pantothenate kinase-associated neurodegeneration (PANK), spinal degenerative disease/motor neuron degenerative diseases, upper motor neuron disorder, lower motor neuron disorder, age-related disorders and dementias, Hallervorden-Spatz syndrome, Lytigo-bodig (amyotrophic lateral sclerosis-parkinsonism dementia), Guam-Parkinsonism dementia, hippocampal sclerosis, corticobasal degeneration, Alexander disease, Apler's disease, Krabbe's disease, neuroborreliosis, neurosyphilis, Sandhoff disease, Schilder's disease, Batten disease, Cockayne syndrome, Kearns-Sayre syndrome, Gerstmann-Straussler-Scheinker syndrome, hereditary spastic paraparesis, Leigh's syndrome, demyelinating diseases, epilepsy, tremors, depression, mania, anxiety and anxiety disorders, sleep disorders (e.g., narcolepsy, fatal familial insomnia), acute brain injuries (e.g., stroke, head injury) and autism. As used herein, the term “α-synucleopathy” refers to a neurodegenerative disorder or disease involving aggregation of α-synuclein or abnormal α-synuclein in nerve cells in the brain (Ostrerova, N., et al. (1999) J Neurosci 19:5782:5791; Rideout, H. J., et al. (2004) J Biol Chem 279:46915-46920). α-Synucleopathies include, but are not limited to, Parkinson's disease, Parkinson's disease with dementia, dementia with Lewy bodies, Pick's disease, Down's syndrome, multiple system atrophy, amylotrophic lateral sclerosis (ALS), Hallervorden-Spatz syndrome, and the like.

As used herein, the term “tauopathy” refers to a neurodegenerative disease associated with the pathological aggregation of tau protein in the brain. Tauopathies include, but are not limited to, Alzheimer's disease, Pick's disease, corticobasal degeneration, Argyrophilic grain disease (AGD), progressive supranuclear palsy, Frontotemporal dementia, Frontotemporal lobar degeneration, or Pick's complex.

Musculoskeletal Diseases:

Musculoskeletal diseases and disorders as defined herein are conditions that affect the muscles, ligaments, tendons, and joints, as well as the skeletal structures that support them. Without wishing to be bound by a theory, aberrant expression of certain proteins, such as the full-length isoform of DUX4, has been shown to inhibit protein turnover and increase the expression and aggregation of cytotoxic proteins including insoluble TDP-43 in skeletal muscle cells (Homma, S. et al. Ann Clin Transl Neurol (2015) 2:151-166).

As such, compounds of Formula (I), Formula (II), and Formula (III) may be used to prevent or treat a musculoskeletal disease, e.g., a musculoskeletal disease that results in accumulation of TDP-43 and other stress granule proteins, e.g., in the nucleus, cytoplasm, or cell bodies of a muscle cell or motor neuron. Exemplary musculoskeletal diseases include muscular dystrophy, facioscapulohumeral muscular dystrophy (e.g., FSHD1 or FSHD2), Freidrich's ataxia, progressive muscular atrophy (PMA), mitochondrial encephalomyopathy (MELAS), multiple sclerosis, inclusion body myopathy, inclusion body myositis (e.g., sporadic inclusion body myositis), post-polio muscular atrophy (PPMA), motor neuron disease, myotonia, myotonic dystrophy, sacropenia, spasticity, multifocal motor neuropathy, inflammatory myopathies, paralysis, and other diseases or disorders relating to the aberrant expression of TDP-43 and altered proteostasis. In addition, compounds of Formula (I) may be used to prevent or treat symptoms caused by or relating to said musculoskeletal diseases, e.g., kyphosis, hypotonia, foot drop, motor dysfunctions, muscle weakness, muscle atrophy, neuron loss, muscle cramps, altered or aberrant gait, dystonias, astrocytosis (e.g., astrocytosis in the spinal cords), liver disease, inflammation, headache, pain (e.g., back pain, neck pain, leg pain, inflammatory pain), and the like. In some embodiments, a musculoskeletal disease or a symptom of a musculoskeletal disease may overlap with a neurodegenerative disease or a symptom of a neurodegenerative disease.

Cancers:

Cancer cells grow quickly and in low oxygen environments by activating different elements of the cellular stress response. Researchers have shown that drugs targeting different elements of the stress response can be anti-neoplastic. For example, rapamycin blocks mTOR, upregulates autophagy and inhibits some types of tumors. Proteasomal inhibitors, such as velcade (Millenium Pharma) are used to treat some cancers. HSP90 inhibitors, such as 17-allylaminogeldanamycin (17AAG), are currently in clinical trials for cancer. Without wishing to be bound by a theory, compounds of Formula (I) may also be used for treatment of cancer, as a greater understanding of the role of TDP-43 in RNA processing and transcription factor signaling has recently begun to emerge (Lagier-Tourenne, C., et al. (2010) Hum Mol Genet 19:R46-R64; Ayala, Y. M., et al. (2008) Proc Natl Acad Sci U.S.A. 105(10):3785-3789). Additionally, TDP-43 modulators can be combined with one or more cancer therapies, such as chemotherapy and radiation therapy.

A “cancer” in a subject refers to the presence of cells possessing characteristics typical of cancer-causing cells, such as uncontrolled proliferation, immortality, metastatic potential, rapid growth and proliferation rate, and certain characteristic morphological features. Often, cancer cells will be in the form of a tumor, but such cells may exist alone within an animal, or may be a non-tumorigenic cancer cell, such as a leukemia cell. In some circumstances, cancer cells will be in the form of a tumor; such cells may exist locally within an animal, or circulate in the blood stream as independent cells, for example, leukemic cells. Examples of cancer include but are not limited to breast cancer, a melanoma, adrenal gland cancer, biliary tract cancer, bladder cancer, brain or central nervous system cancer, bronchus cancer, blastoma, carcinoma, a chondrosarcoma, cancer of the oral cavity or pharynx, cervical cancer, colon cancer, colorectal cancer, esophageal cancer, gastrointestinal cancer, glioblastoma, hepatic carcinoma, hepatoma, kidney cancer, leukemia, liver cancer, lung cancer, lymphoma, non-small cell lung cancer, ophthalmological cancer, osteosarcoma, ovarian cancer, pancreas cancer, peripheral nervous system cancer, prostate cancer, sarcoma, salivary gland cancer, small bowel or appendix cancer, small-cell lung cancer, squamous cell cancer, stomach cancer, testis cancer, thyroid cancer, urinary bladder cancer, uterine or endometrial cancer, vulval cancer, and the like.

Other exemplary cancers include, but are not limited to, ACTH-producing tumors, acute lymphocytic leukemia, acute nonlymphocytic leukemia, cancer of the adrenal cortex, bladder cancer, brain cancer, breast cancer, cervical cancer, chronic lymphocytic leukemia, chronic myelocytic leukemia, colorectal cancer, cutaneous T-cell lymphoma, endometrial cancer, esophageal cancer, Ewing's sarcoma, gallbladder cancer, hairy cell leukemia, head & neck cancer, ophthalmological cancer, Hodgkin's lymphoma, Kaposi's sarcoma, kidney cancer, liver cancer, lung cancer (small and/or non-small cell), malignant peritoneal effusion, malignant pleural effusion, melanoma, mesothelioma, multiple myeloma, neuroblastoma, non-Hodgkin's lymphoma, osteosarcoma, ovarian cancer, ovary (germ cell) cancer, prostate cancer, pancreatic cancer, penile cancer, retinoblastoma, skin cancer, soft-tissue sarcoma, squamous cell carcinomas, stomach cancer, testicular cancer, thyroid cancer, trophoblastic neoplasms, uterine cancer, vaginal cancer, cancer of the vulva, Wilm's tumor, and the like.

Exemplary lymphomas include Hodgkin's lymphoma and non-Hodgkin's lymphoma. Further exemplification of non-Hodgkin's lymphoma include, but are not limited to, B-cell lymphomas (e.g., diffuse large B-cell lymphoma, primary mediastinal B-cell lymphoma, intravascular large B-cell lymphoma, follicular lymphoma, chronic lymphocytic leukemia/small lymphocytic lymphoma, mantle cell lymphoma, marginal zone B-cell lymphomas, extranodal marginal B-cell lymphomas, mucosa-associated lymphoid tissue (MALT) lymphomas, modal marginal zone B-cell lymphoma, splenic marginal zone B-cell lymphoma, Burkitt lymphoma, lymphoplasmacytic lymphoma, Waldenström's macroglobulinemia, hairy cell leukemia, and primary central nervous system (CNS) lymphoma) and T-cell lymphomas (e.g., precursor T-lymphoblastic lymphomalleukemia, peripheral T-cell lymphoma, cutaneous T-cell lymphoma, adult T-cell lymphoma (e.g., smoldering adult T-cell lymphoma, chronic adult T-cell lymphoma, acute adult T-cell lymphoma, lymphomatous adult T-cell lymphoma), angioimmunoblastic T-cell lymphoma, extranodal natural killer T-cell lymphoma nasal type (ENKL), enteropathy-associated intestinal T-cell lymphoma (EATL) (e.g., Type I EATL and Type II EATL), and anaplastic large cell lymphoma (ALCL)).

Ophthalmological Diseases:

Ophthalmological diseases and disorders (e.g., retinal diseases and disorders) as defined herein affect the retina and other parts of the eye and may contribute to impaired vision and blindness. Several ophthalmological diseases (e.g., retinal diseases) are characterized by the accumulation of protein inclusions and stress granules within or between cells of the eye, e.g., retinal cells and nearby tissues. In addition, an ophthalmological disease (e.g., retinal disease) may also be a symptom of or precursor to neurogenerative diseases, such as ALS and FTD (Ward, M. E., et al. (2014) J Exp Med 211(10):1937). Therefore, use of compounds that may inhibit formation of protein inclusions and stress granules, including compounds of Formula (I), may play an important role in the prevention or treatment of ophthalmological diseases (e.g., retinal diseases).

Exemplary ophthalmological diseases (e.g., retinal diseases) include, but are not limited to, macular degeneration (e.g., age-related macular degeneration), diabetes retinopathy, histoplasmosis, macular hole, macular pucker, Bietti's crystalline dystrophy, retinal detachment, retinal thinning, retinoblastoma, retinopathy of prematurity, Usher's syndrome, vitreous detachment, Refsum disease, retinitis pigmentosa, onchocerciasis, choroideremia, Leber congenital amaurosis, retinoschisis (e.g., juvenile retinoschisis), Stargardt disease, ophthalmoplegia, and the like.

Viral Infections:

Stress granules often form during viral illnesses, as viral infections often involve hijacking the cellular reproductive machinery toward production of viral proteins.

In this case, inhibitors of stress granules can be useful for interfering with viral function. Other viruses appear to inhibit SG formation to prevent the cell from mobilizing a stress response. In such a case, an inducer of stress granules can interfere with viral activity and help combat viral infections (e.g., Salubrinal, an eIF2a phosphatase inhibitor and stress granule inducer). Two viruses for which SG biology has been investigated include West Nile virus and respiratory syncytial virus (RSV) (Emara, M. E. and Brinton, M. A. (2007) Proc. Natl. Acad. Sci. USA 104(21): 9041-9046). Therefore, use of compounds that may inhibit formation of protein inclusions and stress granules, including compounds of Formula (I), may be useful for the prevention and/or treatment of a viral infection.

Exemplary viruses include, but are not limited to, West Nile virus, respiratory syncytial virus (RSV), Epstein-Barr virus (EBV), hepatitis A, B, C, and D viruses, herpes viruses, influenza viruses, chicken pox, avian flu viruses, smallpox, polio viruses, HIV, Ebola virus, and the like.

### Imaging

The compounds described herein are useful for detection and/or diagnosis of stress granules. Accordingly, they can be used as in vivo imaging agents of tissues and organs in various biomedical applications. When used in imaging applications, the compounds described herein typically comprise an imaging agent, which can be covalently or noncovalently attached to the compound.

As used herein, the term “imaging agent” refers to an element or functional group in a molecule that allows for the detection, imaging, and/or monitoring of the presence and/or progression of a condition(s), pathological disorder(s), and/or disease(s). The imaging agent may be an echogenic substance (either liquid or gas), non-metallic isotope, an optical reporter, a boron neutron absorber, a paramagnetic metal ion, a ferromagnetic metal, a gamma-emitting radioisotope, a positron-emitting radioisotope, or an x-ray absorber.

Suitable optical reporters include, but are not limited to, fluorescent reporters and chemiluminescent groups. A wide variety of fluorescent reporter dyes are known in the art. Typically, the fluorophore is an aromatic or heteroaromatic compound and can be a pyrene, anthracene, naphthalene, acridine, stilbene, indole, benzindole, oxazole, thiazole, benzothiazole, cyanine, carbocyanine, salicylate, anthranilate, coumarin, fluorescein, rhodamine or other like compound. Suitable fluorescent reporters include xanthene dyes, such as fluorescein or rhodamine dyes, including, but not limited to, Alexa Fluor® dyes (InvitrogenCorp.; Carlsbad, Calif.), fluorescein, fluorescein isothiocyanate (FITC), Oregon Green™, rhodamine, Texas red, tetrarhodamine isothiocynate (TRITC), 5-carboxyfluorescein (FAM), 2′7′-dimethoxy-4′5′-dichloro-6-carboxyfluorescein (JOE), tetrachlorofluorescein (TET), 6-carboxyrhodamine (R6G), N,N,N,N′-tetramefhyl-6-carboxyrhodamine (TAMRA), and 6-carboxy-X-rhodamine (ROX). Suitable fluorescent reporters also include the naphthylamine dyes that have an amino group in the alpha or beta position. For example, naphthylamino compounds include 1-dimethylamino-naphthyl-5-sulfonate, 1-anilino-8-naphthalene sulfonate, 2-p-toluidinyl-6-naphthalene sulfonate, and 5-(2′-aminoethyl)aminonaphthalene-1-sulfonic acid (EDANS). Other fluorescent reporter dyes include coumarins, such as 3-phenyl-7-isocyanatocoumarin; acridines, such as 9-isothiocyanatoacridine and acridine orange; N-(p(2-benzoxazolyl)phenyl)maleimide; cyanines, such as Cy2, indodicarbocyanine 3 (Cy3), indodicarbocyanine 5 (Cy5), indodicarbocyanine 5.5 (Cy5.5), 3-(-carboxy-pentyl)-3′ethyl-5,5′-dimethyloxacarbocyanine (CyA); 1H,5H,11H,15H-xantheno[2,3,4-ij:5,6,7-i′j′]diquinolizin-18-ium, 9-[2(or 4)-[[[6-[2,5-dioxo-1-pyrrolidinyl)oxy]-6-oxohexyl] amino]sulfonyl]-4(or 2)-sulfophenyl]-2,3,6,7,12,13,16,17-octahydro-inner salt (TR or Texas Red); BODIPY™ dyes; benzoxadiazoles; stilbenes; pyrenes; and the like. Many suitable forms of these fluorescent compounds are available and can be used as labels.

Examples of fluorescent proteins suitable for use as imaging agents include, but are not limited to, green fluorescent protein, red fluorescent protein (e.g., DsRed), yellow fluorescent protein, cyan fluorescent protein, blue fluorescent protein, and variants thereof (see, e.g., U.S. Pat. Nos. 6,403,374, 6,800,733, and 7,157,566). Specific examples of GFP variants include, but are not limited to, enhanced GFP (EGFP), destabilized EGFP, the GFP variants described in Doan et al, (2005) Mol Microbiol 55:1767-1781, the GFP variant described in Crameri et al, (1996) Nat Biotechnol 14:315319, the cerulean fluorescent proteins described in Rizzo et al, (2004) Nat Biotechnol, 22:445 and Tsien, (1998) Annu Rev Biochem 67:509, and the yellow fluorescent protein described in Nagal et al, (2002) Nat Biotechnol 20:87-90. DsRed variants are described in, e.g., Shaner et al, (2004) Nat Biotechnol 22:1567-1572, and include mStrawberry, mCherry, mOrange, mBanana, mHoneydew, and mTangerine. Additional DsRed variants are described in, e.g., Wang et al, (2004) Proc Natl Acad Sci U.S.A. 101:16745-16749, and include mRaspberry and mPlum. Further examples of DsRed variants include mRFPmars described in Fischer et al, (2004) FEBS Lett 577:227-232 and mRFPruby described in Fischer et al, (2006) FEBS Lett 580:2495-2502.

Suitable echogenic gases include, but are not limited to, a sulfur hexafluoride or perfluorocarbon gas, such as perfluoromethane, perfluoroethane, perfluoropropane, perfluorobutane, perfluorocyclobutane, perfluropentane, or perfluorohexane.

Suitable non-metallic isotopes include, but are not limited to, ¹¹C, ¹⁴C, ¹³N, ¹⁸F, ¹²³I, ¹²⁴I, and ¹²⁵I.

Suitable radioisotopes include, but are not limited to, ⁹⁹mTc, ⁹⁵Tc, ¹¹¹In, ⁶²Cu, ⁶⁴Cu, Ga, ⁶⁸Ga, and ¹⁵³Gd.

Suitable paramagnetic metal ions include, but are not limited to, Gd(III), Dy(III), Fe(III), and Mn(II).

Suitable X-ray absorbers include, but are not limited to, Re, Sm, Ho, Lu, Pm, Y, Bi, Pd, Gd, La, Au, Au, Yb, Dy, Cu, Rh, Ag, and Ir.

In some embodiments, the radionuclide is bound to a chelating agent or chelating agent-linker attached to the aggregate. Suitable radionuclides for direct conjugation include, without limitation, ¹⁸F, ¹²⁴I, ¹²⁵I, ¹³¹I, and mixtures thereof. Suitable radionuclides for use with a chelating agent include, without limitation, ⁴⁷Sc, ⁶⁴Cu, ⁶⁷Cu, ⁸⁹Sr, ⁸⁶Y, ⁸⁷Y, ⁹⁰Y, ¹⁰⁵Rh, ¹¹¹Ag, ¹¹In, ¹¹⁷mSn, ¹⁴⁹Pm, ¹⁵³Sm, ¹⁶⁶Ho, ¹⁷⁷Lu, ¹⁸⁶Re, ¹⁸⁸Re, ²¹¹At, ²¹²Bi, and mixtures thereof. Suitable chelating agents include, but are not limited to, DOTA, BAD, TETA, DTPA, EDTA, NTA, HDTA, their phosphonate analogs, and mixtures thereof. One of skill in the art will be familiar with methods for attaching radionuclides, chelating agents, and chelating agent-linkers to the aggregate or small molecule.

A detectable response generally refers to a change in, or occurrence of, a signal that is detectable either by observation or instrumentally. In certain instances, the detectable response is fluorescence or a change in fluorescence, e.g., a change in fluorescence intensity, fluorescence excitation or emission wavelength distribution, fluorescence lifetime, and/or fluorescence polarization. One of skill in the art will appreciate that the degree and/or location of labeling in a subject or sample can be compared to a standard or control (e.g., healthy tissue or organ). In certain other instances, the detectable response the detectable response is radioactivity (i.e., radiation), including alpha particles, beta particles, nucleons, electrons, positrons, neutrinos, and gamma rays emitted by a radioactive substance such as a radionuclide.

Specific devices or methods known in the art for the in vivo detection of fluorescence, e.g., from fluorophores or fluorescent proteins, include, but are not limited to, in vivo near-infrared fluorescence (see, e.g., Frangioni, (2003) Curr Opin Chem Biol 7:626-634), the Maestro™ in vivo fluorescence imaging system (Cambridge Research & Instrumentation, Inc.; Woburn, Mass.), in vivo fluorescence imaging using a flying-spot scanner (see, e.g., Ramanujam et al, (2001) IEEE Transactions on Biomedical Engineering, 48:1034-1041, Other methods or devices for detecting an optical response include, without limitation, visual inspection, CCD cameras, video cameras, photographic film, laser-scanning devices, fluorometers, photodiodes, quantum counters, epifluorescence microscopes, scanning microscopes, flow cytometers, fluorescence microplate readers, or signal amplification using photomultiplier tubes.

Any device or method known in the art for detecting the radioactive emissions of radionuclides in a subject is suitable for use in the present invention. For example, methods such as Single Photon Emission Computerized Tomography (SPECT), which detects the radiation from a single photon gamma-emitting radionuclide using a rotating gamma camera, and radionuclide scintigraphy, which obtains an image or series of sequential images of the distribution of a radionuclide in tissues, organs, or body systems using a scintillation gamma camera, may be used for detecting the radiation emitted from a radiolabeled aggregate. Positron emission tomography (PET) is another suitable technique for detecting radiation in a subject.

Magnetic resonance imaging (MRI), nuclear magnetic resonance imaging (NMRI), or magnetic resonance tomography (MRT) is a medical imaging technique used in radiology to visualize detailed internal structures. MRI makes use of the property of nuclear magnetic resonance (NMR) to image nuclei of atoms inside the body. Thus, labels having magnetic properties can be detected using MRI and/or related technologies.

SG proteins, such as TDP-43, undergo translocation to the cytoplasm and may form aggregates. Translocation likely requires a post-translational modification as well as binding to a transport protein. Aggregation is often associated with a change in protein conformation. Modulators of TDP-43 can bind to SG proteins specifically under states of cytoplasmic translocation (for instance, because they recognize a binding site enabled by a post-translational modification) or SG proteins that are in an aggregated state associated with SGs. Thus, modulators of TDP-43 inclusions can be used to image areas in a subject's body that have increased levels of SGs, either physiological or pathological. For instance, in ALS and Alzheimer's disease, the inventors have demonstrated that TDP-43 associates with the pathological form of TDP-43 that accumulates. Thus, compounds that recognize aggregated TDP-43 can be used to image pathology, much like the imaging agent PiB, which is currently used in Alzheimer's research. However, a drawback to use of PiB in imaging protein aggregates is that it recognizes amyloid protein, which accumulates both in patients with Alzheimer's disease and in many non-affected people. However, an agent that recognizes SGs would specifically target patients that have demonstrated intracellular pathology, such as neurofibrillary tangles, which are associated with SGs. Such agents can be used to diagnose patients at risk of developing a neurodegenerative illness.

Additionally, imaging of SGs in a subject can be used to localize pain. For example, a compound of Formula (I) can be administered to a subject experiencing pain, wherein the pain is difficult to localize. Subsequent imaging may be used to localize the area of the body exhibiting this pain, revealing disease or injury. This can greatly speed diagnosis and can be generally applicable throughout the medical arts.

Further, the compounds described herein can be used to image organs for transplants. Organs are harvested for transplants, such as kidneys and hearts. A problem in the field is that it is unclear to medical professionals how well the organ survived the harvesting and transport to the receiving hospital. Sometimes, organs are transplanted only to have them fail because they were injured in transport. A quick cytologic stain with a stress granule marker would represent a large advance for the field. Accordingly, compound of Formula (I) may be used as in the analysis of organs for transplantation.

### Definitions

Unless stated otherwise, or implicit from context, the following terms and phrases include the meanings provided below. Unless explicitly stated otherwise, or apparent from context, the terms and phrases below do not exclude the meaning that the term or phrase has acquired in the art to which it pertains. The definitions are provided to aid in describing particular embodiments, and are not intended to limit the claimed invention, because the scope of the invention is limited only by the claims. Further, unless otherwise required by context, singular terms shall include pluralities and plural terms shall include the singular.

As used herein, the terms “compounds” and “agent” are used interchangeably to refer to the inhibitors/antagonists/agonists of the invention. In certain embodiments, the compounds are small organic or inorganic molecules, e.g., with molecular weights less than 7500 amu, preferably less than 5000 amu, and even more preferably less than 2000, 1500, 1000, 750, 600, or 500 amu. In certain embodiments, one class of small organic or inorganic molecules are non-peptidyl, e.g., containing 2, 1, or no peptide and/or saccharide linkages.

Unless otherwise indicated, all numbers expressing quantities of ingredients or reaction conditions used herein should be understood as modified in all instances by the term “about.” The term “about” when used in connection with percentages may mean±1%.

The singular terms “a,” “an,” and “the” refer to one or to more than one, unless context clearly indicates otherwise. Similarly, the word “or” is intended to include “and” unless the context clearly indicates otherwise.

Although methods and materials similar or equivalent to those described herein can be used in the practice or testing of this disclosure, suitable methods and materials are described below. The term “comprises” means “includes.” The abbreviation, “e.g.” is derived from the Latin exempli gratia, and is used herein to indicate a non-limiting example. Thus, the abbreviation “e.g.” is synonymous with the term “for example.”

The terms “decrease”, “reduced”, “reduction”, “decrease” or “inhibit” are all used herein generally to mean a decrease by a statistically significant amount. However, for avoidance of doubt, “reduced”, “reduction”, “decrease” or “inhibit” means a decrease by at least 1% as compared to a reference level, for example a decrease by at least about 5%, or at least about 10%, or at least about 15%, or at least about 20%, or at least about 30%, or at least about 40%, or at least about 50%, or at least about 60%, or at least about 70%, or at least about 80%, or at least about 90% or up to and including a 100% decrease (e.g. absent level as compared to a reference sample), or any decrease between 1-100%, e.g., 10-100% as compared to a reference level.

The terms “increased”, “increase”, “enhance” or “activate” are all used herein to generally mean an increase by a statically significant amount; for the avoidance of any doubt, the terms “increased”, “increase”, “enhance” or “activate” means an increase by at least 1% as compared to a reference level, for example a decrease by at least about 5%, or at least about 10%, or at least about 15%, or at least about 20%, or at least about 30%, or at least about 40%, or at least about 50%, or at least about 60%, or at least about 70%, or at least about 80%, or at least about 90% or up to and including a 100% increase (e.g. absent level as compared to a reference sample), or any increase between 1-100%, e.g., 10-100% as compared to a reference level.

As used herein, the term “administer” refers to the placement of a composition into a subject by a method or route which results in at least partial localization of the composition at a desired site such that desired effect is produced. A compound or composition described herein can be administered by any appropriate route known in the art including, but not limited to, oral or parenteral routes, including intravenous, intramuscular, subcutaneous, transdermal, airway (aerosol), pulmonary, nasal, rectal, intrathecal, and topical (including buccal and sublingual) administration.

Exemplary modes of administration include, but are not limited to, injection, infusion, instillation, inhalation, or ingestion. “Injection” includes, without limitation, intravenous, intramuscular, intraarterial, intrathecal, intraventricular, intracapsular, intraorbital, intracardiac, intradermal, intraperitoneal, transtracheal, subcutaneous, subcuticular, intraarticular, sub capsular, subarachnoid, intraspinal, intracerebro spinal, and intrasternal injection and infusion. In some embodiments, the compositions are administered by intravenous infusion or injection.

By “treatment”, “prevention” or “amelioration” of a disease or disorder is meant delaying or preventing the onset of such a disease or disorder, reversing, alleviating, ameliorating, inhibiting, slowing down or stopping the progression, aggravation or deterioration the progression or severity of a condition associated with such a disease or disorder. In one embodiment, at least one symptom of a disease or disorder is alleviated by at least 5%, at least 10%, at least 20%, at least 30%, at least 40%, or at least 50%.

As used herein, an amount of a compound or combination effective to treat a disorder (e.g., a disorder as described herein), “therapeutically effective amount”, “effective amount” or “effective course” refers to an amount of the compound or combination which is effective, upon single or multiple dose administration(s) to a subject, in treating a subject, or in curing, alleviating, relieving or improving a subject with a disorder (e.g., a disorder as described herein) beyond that expected in the absence of such treatment. Determination of a therapeutically effective amount is well within the capability of those skilled in the art. Generally, a therapeutically effective amount can vary with the subject's history, age, condition, sex, as well as the severity and type of the medical condition in the subject, and administration of other pharmaceutically active agents.

As used herein, a “subject” means a human or animal. Usually the animal is a vertebrate such as a primate, rodent, domestic animal or game animal. Primates include chimpanzees, cynomologous monkeys, spider monkeys, and macaques, e.g., Rhesus. Rodents include mice, rats, woodchucks, ferrets, rabbits and hamsters. Domestic and game animals include cows, horses, pigs, deer, bison, buffalo, feline species, e.g., domestic cat, canine species, e.g., dog, fox, wolf, avian species, e.g., chicken, emu, ostrich, and fish, e.g., trout, catfish and salmon. Patient or subject includes any subset of the foregoing, e.g., all of the above, but excluding one or more groups or species such as humans, primates or rodents. In certain embodiments, the subject is a mammal, e.g., a primate, e.g., a human. The terms, “patient” and “subject” are used interchangeably herein. The terms, “patient” and “subject” are used interchangeably herein. The term “nucleic acid” as used herein refers to a polymeric form of nucleotides, either ribonucleotides or deoxynucleotides or a modified form of either type of nucleotide. The terms should also be understood to include, as equivalents, analogs of either RNA or DNA made from nucleotide analogs, and, as applicable to the embodiment being described, single-stranded (such as sense or antisense) and double-stranded polynucleotides.

As used herein, the terms “modulator of stress granule” and “stress granule modulator” refer to compounds and compositions of Formula (I) that modulate the formation and/or disaggregation of stress granules.

The term “TDP-43 inclusion” as used herein refers to protein-mRNA aggregates that comprise a TDP-43 protein. The TDP-43 protein in a stress granule can be wild-type or a mutant form of TDP-43.

As used herein, the terms “modulator of TDP-43 inclusion” and “TDP-43 inclusion modulator” refer to compounds and compositions of Formula (I) and Formula (II) that modulate the formation and/or disaggregation of cytoplasmic TDP-43 inclusions.

### Selected Chemical Definitions

At various places in the present specification, substituents of compounds of the invention are disclosed in groups or in ranges. It is specifically intended that the invention include each and every individual subcombination of the members of such groups and ranges. For example, the term “C₁₋₆ alkyl” is specifically intended to individually disclose methyl, ethyl, propyl, butyl, and pentyl.

For compounds of the invention in which a variable appears more than once, each variable can be a different moiety selected from the Markush group defining the variable. For example, where a structure is described having two R groups that are simultaneously present on the same compound; the two R groups can represent different moieties selected from the Markush group defined for R.

It is further appreciated that certain features of the invention, which are, for clarity, described in the context of separate embodiments, can also be provided in combination in a single embodiment. Conversely, various features of the invention which are, for brevity, described in the context of a single embodiment, can also be provided separately or in any suitable subcombination.

If a compound of the present invention is depicted in the form of a chemical name and as a formula, in case of any discrepancy, the formula shall prevail.

The symbol [symbol] , whether utilized as a bond or displayed perpendicular to a bond indicates the point at which the displayed moiety is attached to the remainder of the molecule, solid support, etc.

The following terms are intended to have the meanings presented therewith below and are useful in understanding the description and intended scope of the present invention.

As used herein, “alkyl” refers to a radical of a straight-chain or branched saturated hydrocarbon group having from 1 to 24 carbon atoms (“C₁-C₂₄ alkyl”). In some embodiments, an alkyl group has 1 to 12 carbon atoms (“C₁-C₁₂ alkyl”). In some embodiments, an alkyl group has 1 to 8 carbon atoms (“C₁-C₈ alkyl”). In some embodiments, an alkyl group has 1 to 6 carbon atoms (“C₁-C₆ alkyl”). In some embodiments, an alkyl group has 1 to 5 carbon atoms (“C₁-C₅ alkyl”). In some embodiments, an alkyl group has 1 to 4 carbon atoms (“C₁-C₄alkyl”). In some embodiments, an alkyl group has 1 to 3 carbon atoms (“C₁-C₃ alkyl”). In some embodiments, an alkyl group has 1 to 2 carbon atoms (“C₁-C₂ alkyl”). In some embodiments, an alkyl group has 1 carbon atom (“C₁ alkyl”). In some embodiments, an alkyl group has 2 to 6 carbon atoms (“C₂-C₆alkyl”). Examples of C₁-C₆alkyl groups include methyl (C₁), ethyl (C₂), n-propyl (C₃), isopropyl (C₃), n-butyl (C₄), tert-butyl (C₄), sec-butyl (C₄), iso-butyl (C₄), n-pentyl (C₅), 3-pentanyl (C₅), amyl (C₅), neopentyl (C₅), 3-methyl-2-butanyl (C₅), tertiary amyl (C₅), and n-hexyl (C₆). Additional examples of alkyl groups include n-heptyl (C₇), n-octyl (C₈) and the like.

Each instance of an alkyl group may be independently optionally substituted, i.e., unsubstituted (an “unsubstituted alkyl”) or substituted (a “substituted alkyl”) with one or more substituents; e.g., for instance from 1 to 5 substituents, 1 to 3 substituents, or 1 substituent. In certain embodiments, the alkyl group is unsubstituted C₁₋₁₀ alkyl (e.g., —CH₃). In certain embodiments, the alkyl group is substituted C₁₋₆ alkyl.

As used herein, “alkenyl” refers to a radical of a straight-chain or branched hydrocarbon group having from 2 to 24 carbon atoms, one or more carbon-carbon double bonds, and no triple bonds (“C₂-C₂₄ alkenyl”). In some embodiments, an alkenyl group has 2 to 10 carbon atoms (“C₂-C₁₀ alkenyl”). In some embodiments, an alkenyl group has 2 to 8 carbon atoms (“C₂-C₈ alkenyl”). In some embodiments, an alkenyl group has 2 to 6 carbon atoms (“C₂-C₆ alkenyl”).

In some embodiments, an alkenyl group has 2 to 5 carbon atoms (“C₂-C₅ alkenyl”). In some embodiments, an alkenyl group has 2 to 4 carbon atoms (“C₂-C₄ alkenyl”). In some embodiments, an alkenyl group has 2 to 3 carbon atoms (“C₂-C₃ alkenyl”). In some embodiments, an alkenyl group has 2 carbon atoms (“C₂ alkenyl”). The one or more carbon-carbon double bonds can be internal (such as in 2-butenyl) or terminal (such as in 1-butenyl). Examples of C₂-C₄ alkenyl groups include ethenyl (C₂), 1-propenyl (C₃), 2-propenyl (C₃), 1-butenyl (C₄), 2-butenyl (C₄), butadienyl (C₄), and the like. Examples of C₂-C₆ alkenyl groups include the aforementioned C₂₋₄ alkenyl groups as well as pentenyl (C₅), pentadienyl (C₅), hexenyl (C₆), and the like. Additional examples of alkenyl include heptenyl (C₇), octenyl (C₈), octatrienyl (C₈), and the like. Each instance of an alkenyl group may be independently optionally substituted, i.e., unsubstituted (an “unsubstituted alkenyl”) or substituted (a “substituted alkenyl”) with one or more substituents e.g., for instance from 1 to 5 substituents, 1 to 3 substituents, or 1 substituent. In certain embodiments, the alkenyl group is unsubstituted C₂₋₁₀ alkenyl. In certain embodiments, the alkenyl group is substituted C₂₋₆ alkenyl.

As used herein, the term “alkynyl” refers to a radical of a straight-chain or branched hydrocarbon group having from 2 to 24 carbon atoms, one or more carbon-carbon triple bonds (“C₂-C₂₄ alkenyl”). In some embodiments, an alkynyl group has 2 to 10 carbon atoms (“C₂-C₁₀ alkynyl”). In some embodiments, an alkynyl group has 2 to 8 carbon atoms (“C₂-C₈ alkynyl”). In some embodiments, an alkynyl group has 2 to 6 carbon atoms (“C₂-C₆ alkynyl”). In some embodiments, an alkynyl group has 2 to 5 carbon atoms (“C₂-C₅ alkynyl”). In some embodiments, an alkynyl group has 2 to 4 carbon atoms (“C₂-C₄ alkynyl”). In some embodiments, an alkynyl group has 2 to 3 carbon atoms (“C₂-C₃ alkynyl”). In some embodiments, an alkynyl group has 2 carbon atoms (“C₂ alkynyl”). The one or more carbon-carbon triple bonds can be internal (such as in 2-butynyl) or terminal (such as in 1-butynyl). Examples of C₂-C₄ alkynyl groups include ethynyl (C₂), 1-propynyl (C₃), 2-propynyl (C₃), 1-butynyl (C₄), 2-butynyl (C₄), and the like. Each instance of an alkynyl group may be independently optionally substituted, i.e., unsubstituted (an “unsubstituted alkynyl”) or substituted (a “substituted alkynyl”) with one or more substituents e.g., for instance from 1 to 5 substituents, 1 to 3 substituents, or 1 substituent. In certain embodiments, the alkynyl group is unsubstituted C₂₋₁₀ alkynyl. In certain embodiments, the alkynyl group is substituted C₂-alkynyl.

As used herein, the term “heteroalkyl,” refers to a non-cyclic stable straight or branched chain, or combinations thereof, including at least one carbon atom and at least one heteroatom selected from the group consisting of O, N, P, Si, and S, and wherein the nitrogen and sulfur atoms may optionally be oxidized, and the nitrogen heteroatom may optionally be quaternized.

The heteroatom(s) 0, N, P, S, and Si may be placed at any position of the heteroalkyl group.

Exemplary heteroalkyl groups include, but are not limited to: —CH₂—CH₂—O—CH₃, —CH₂—CH₂—NH—CH₃, —CH₂—CH₂—N(CH₃)—CH₃, —CH₂—S—CH₂—CH₃, —CH₂—CH₂, —S(O)—CH₃, —CH₂—CH₂—S(O)₂—CH₃, —CH═CH—O—CH₃, —Si(CH₃)₃, —CH₂—CH═N—OCH₃, —CH═CH—N(CH₃)—CH₃, —O—CH₃, and —O—CH₂—CH₃. Up to two or three heteroatoms may be consecutive, such as, for example, —CH₂—NH—OCH₃ and —CH₂—O—Si(CH₃)₃. Where “heteroalkyl” is recited, followed by recitations of specific heteroalkyl groups, such as —CH₂O, —NR^{C}R^{D}, or the like, it will be understood that the terms heteroalkyl and —CH₂O or —NR^{C}R^{D} are not redundant or mutually exclusive. Rather, the specific heteroalkyl groups are recited to add clarity. Thus, the term “heteroalkyl” should not be interpreted herein as excluding specific heteroalkyl groups, such as —CH₂O, —NR^{C}R^{D}, or the like.

The terms “alkylene,” “alkenylene,” “alkynylene,” or “heteroalkylene,” alone or as part of another substituent, mean, unless otherwise stated, a divalent radical derived from an alkyl, alkenyl, alkynyl, or heteroalkyl, respectively. The term “alkenylene,” by itself or as part of another substituent, means, unless otherwise stated, a divalent radical derived from an alkene. An alkylene, alkenylene, alkynylene, or heteroalkylene group may be described as, e.g., a C₁-C₆-membered alkylene, C₁-C₆-membered alkenylene, C₁-C₆-membered alkynylene, or C₁-C₆-membered heteroalkylene, wherein the term “membered” refers to the non-hydrogen atoms within the moiety. In the case of heteroalkylene groups, heteroatoms can also occupy either or both of the chain termini (e.g., alkyleneoxy, alkylenedioxy, alkyleneamino, alkylenediamino, and the like). Still further, for alkylene and heteroalkylene linking groups, no orientation of the linking group is implied by the direction in which the formula of the linking group is written. For example, the formula —C(O)₂R′— may represent both —C(O)₂R′— and —R′C(O)₂—.

As used herein, “aryl” refers to a radical of a monocyclic or polycyclic (e.g., bicyclic or tricyclic) 4n+2 aromatic ring system (e.g., having 6, 10, or 14 it electrons shared in a cyclic array) having 6-14 ring carbon atoms and zero heteroatoms provided in the aromatic ring system (“C₆-C₁₄ aryl”). In some embodiments, an aryl group has six ring carbon atoms (“C₆ aryl”; e.g., phenyl). In some embodiments, an aryl group has ten ring carbon atoms (“C₁₀ aryl”; e.g., naphthyl such as 1-naphthyl and 2-naphthyl). In some embodiments, an aryl group has fourteen ring carbon atoms (“C₁₄ aryl”; e.g., anthracyl). An aryl group may be described as, e.g., a C₆-C₁₀-membered aryl, wherein the term “membered” refers to the non-hydrogen ring atoms within the moiety. Aryl groups include phenyl, naphthyl, indenyl, and tetrahydronaphthyl. Each instance of an aryl group may be independently optionally substituted, i.e., unsubstituted (an “unsubstituted aryl”) or substituted (a “substituted aryl”) with one or more substituents. In certain embodiments, the aryl group is unsubstituted C₆-C₁₄ aryl. In certain embodiments, the aryl group is substituted C₆-C₁₄ aryl.

As used herein, “heteroaryl” refers to a radical of a 5-10 membered monocyclic or bicyclic 4n+2 aromatic ring system (e.g., having 6 or 10 π electrons shared in a cyclic array) having ring carbon atoms and 1-4 ring heteroatoms provided in the aromatic ring system, wherein each heteroatom is independently selected from nitrogen, oxygen and sulfur (“5-10 membered heteroaryl”). In heteroaryl groups that contain one or more nitrogen atoms, the point of attachment can be a carbon or nitrogen atom, as valency permits. Heteroaryl bicyclic ring systems can include one or more heteroatoms in one or both rings. “Heteroaryl” also includes ring systems wherein the heteroaryl ring, as defined above, is fused with one or more aryl groups wherein the point of attachment is either on the aryl or heteroaryl ring, and in such instances, the number of ring members designates the number of ring members in the fused (aryl/heteroaryl) ring system. Bicyclic heteroaryl groups wherein one ring does not contain a heteroatom (e.g., indolyl, quinolinyl, carbazolyl, and the like) the point of attachment can be on either ring, i.e., either the ring bearing a heteroatom (e.g., 2-indolyl) or the ring that does not contain a heteroatom (e.g., 5-indolyl). A heteroaryl group may be described as, e.g., a 6-10-membered heteroaryl, wherein the term “membered” refers to the non-hydrogen ring atoms within the moiety.

In some embodiments, a heteroaryl group is a 5-10 membered aromatic ring system having ring carbon atoms and 1-4 ring heteroatoms provided in the aromatic ring system, wherein each heteroatom is independently selected from nitrogen, oxygen, and sulfur (“5-10 membered heteroaryl”). In some embodiments, a heteroaryl group is a 5-8 membered aromatic ring system having ring carbon atoms and 1-4 ring heteroatoms provided in the aromatic ring system, wherein each heteroatom is independently selected from nitrogen, oxygen, and sulfur (“5-8 membered heteroaryl”). In some embodiments, a heteroaryl group is a 5-6 membered aromatic ring system having ring carbon atoms and 1-4 ring heteroatoms provided in the aromatic ring system, wherein each heteroatom is independently selected from nitrogen, oxygen, and sulfur (“5-6 membered heteroaryl”). In some embodiments, the 5-6 membered heteroaryl has 1-3 ring heteroatoms selected from nitrogen, oxygen, and sulfur. In some embodiments, the 5-6 membered heteroaryl has 1-2 ring heteroatoms selected from nitrogen, oxygen, and sulfur. In some embodiments, the 5-6 membered heteroaryl has 1 ring heteroatom selected from nitrogen, oxygen, and sulfur. Each instance of a heteroaryl group may be independently optionally substituted, i.e., unsubstituted (an “unsubstituted heteroaryl”) or substituted (a “substituted heteroaryl”) with one or more substituents. In certain embodiments, the heteroaryl group is unsubstituted 5-14 membered heteroaryl. In certain embodiments, the heteroaryl group is substituted 5-14 membered heteroaryl.

Exemplary 5-membered heteroaryl groups containing one heteroatom include, without limitation, pyrrolyl, furanyl and thiophenyl. Exemplary 5-membered heteroaryl groups containing two heteroatoms include, without limitation, imidazolyl, pyrazolyl, oxazolyl, isoxazolyl, thiazolyl, and isothiazolyl. Exemplary 5-membered heteroaryl groups containing three heteroatoms include, without limitation, triazolyl, oxadiazolyl, and thiadiazolyl.

Exemplary 5-membered heteroaryl groups containing four heteroatoms include, without limitation, tetrazolyl. Exemplary 6-membered heteroaryl groups containing one heteroatom include, without limitation, pyridinyl. Exemplary 6-membered heteroaryl groups containing two heteroatoms include, without limitation, pyridazinyl, pyrimidinyl, and pyrazinyl. Exemplary 6-membered heteroaryl groups containing three or four heteroatoms include, without limitation, triazinyl and tetrazinyl, respectively. Exemplary 7-membered heteroaryl groups containing one heteroatom include, without limitation, azepinyl, oxepinyl, and thiepinyl. Exemplary 5,6-bicyclic heteroaryl groups include, without limitation, indolyl, isoindolyl, indazolyl, benzotriazolyl, benzothiophenyl, isobenzothiophenyl, benzofuranyl, benzoisofuranyl, benzimidazolyl, benzoxazolyl, benzisoxazolyl, benzoxadiazolyl, benzthiazolyl, benzisothiazolyl, benzthiadiazolyl, indolizinyl, and purinyl. Exemplary 6,6-bicyclic heteroaryl groups include, without limitation, naphthyridinyl, pteridinyl, quinolinyl, isoquinolinyl, cinnolinyl, quinoxalinyl, phthalazinyl, and quinazolinyl. Other exemplary heteroaryl groups include heme and heme derivatives.

As used herein, the terms “arylene” and “heteroarylene,” alone or as part of another substituent, mean a divalent radical derived from an aryl and heteroaryl, respectively.

As used herein, “cycloalkyl” refers to a radical of a non-aromatic cyclic hydrocarbon group having from 3 to 10 ring carbon atoms (“C₃-C₁₀ cycloalkyl”) and zero heteroatoms in the non-aromatic ring system. In some embodiments, a cycloalkyl group has 3 to 8 ring carbon atoms (“C₃-C₈cycloalkyl”). In some embodiments, a cycloalkyl group has 3 to 6 ring carbon atoms (“C₃-C₆ cycloalkyl”). In some embodiments, a cycloalkyl group has 3 to 6 ring carbon atoms (“C₃-C₆ cycloalkyl”). In some embodiments, a cycloalkyl group has 5 to 10 ring carbon atoms (“C₅-C₁₀ cycloalkyl”). A cycloalkyl group may be described as, e.g., a C₄-C₇-membered cycloalkyl, wherein the term “membered” refers to the non-hydrogen ring atoms within the moiety. Exemplary C₃-C₆ cycloalkyl groups include, without limitation, cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), cyclohexadienyl (C₆), and the like. Exemplary C₃-C₈ cycloalkyl groups include, without limitation, the aforementioned C₃-C₆ cycloalkyl groups as well as cycloheptyl (C₇), cycloheptenyl (C₇), cycloheptadienyl (C₇), cycloheptatrienyl (C₇), cyclooctyl (C₈), cyclooctenyl (C₈), cubanyl (C₈), bicyclo[1.1.1]pentanyl (C₅), bicyclo[2.2.2]octanyl (C₈), bicyclo[2.1.1]hexanyl (C₆), bicyclo[3.1.1]heptanyl (C₇), and the like.

Exemplary C₃-C₁₀ cycloalkyl groups include, without limitation, the aforementioned C₃-C₈ cycloalkyl groups as well as cyclononyl (C₉), cyclononenyl (C₉), cyclodecyl (C₁₀), cyclodecenyl (C₁₀), octahydro-1H-indenyl (C₉), decahydronaphthalenyl (C₁₀), spiro[4.5]decanyl (C₁₀), and the like. As the foregoing examples illustrate, in certain embodiments, the cycloalkyl group is either monocyclic (“monocyclic cycloalkyl”) or contain a fused, bridged or spiro ring system such as a bicyclic system (“bicyclic cycloalkyl”) and can be saturated or can be partially unsaturated. “Cycloalkyl” also includes ring systems wherein the cycloalkyl ring, as defined above, is fused with one or more aryl groups wherein the point of attachment is on the cycloalkyl ring, and in such instances, the number of carbons continue to designate the number of carbons in the cycloalkyl ring system. Each instance of a cycloalkyl group may be independently optionally substituted, i.e., unsubstituted (an “unsubstituted cycloalkyl”) or substituted (a “substituted cycloalkyl”) with one or more substituents. In certain embodiments, the cycloalkyl group is unsubstituted C₃-C₁₀ cycloalkyl. In certain embodiments, the cycloalkyl group is a substituted C₃-C₁₀ cycloalkyl.

“Heterocyclyl” as used herein refers to a radical of a 3- to 10-membered non-aromatic ring system having ring carbon atoms and 1 to 4 ring heteroatoms, wherein each heteroatom is independently selected from nitrogen, oxygen, sulfur, boron, phosphorus, and silicon (“3-10 membered heterocyclyl”). In heterocyclyl groups that contain one or more nitrogen atoms, the point of attachment can be a carbon or nitrogen atom, as valency permits. A heterocyclyl group can either be monocyclic (“monocyclic heterocyclyl”) or a fused, bridged or spiro ring system such as a bicyclic system (“bicyclic heterocyclyl”), and can be saturated or can be partially unsaturated. Heterocyclyl bicyclic ring systems can include one or more heteroatoms in one or both rings. “Heterocyclyl” also includes ring systems wherein the heterocyclyl ring, as defined above, is fused with one or more cycloalkyl groups wherein the point of attachment is either on the cycloalkyl or heterocyclyl ring, or ring systems wherein the heterocyclyl ring, as defined above, is fused with one or more aryl or heteroaryl groups, wherein the point of attachment is on the heterocyclyl ring, and in such instances, the number of ring members continue to designate the number of ring members in the heterocyclyl ring system. A heterocyclyl group may be described as, e.g., a 3-7-membered heterocyclyl, wherein the term “membered” refers to the non-hydrogen ring atoms, i.e., carbon, nitrogen, oxygen, sulfur, boron, phosphorus, and silicon, within the moiety. Each instance of heterocyclyl may be independently optionally substituted, i.e., unsubstituted (an “unsubstituted heterocyclyl”) or substituted (a “substituted heterocyclyl”) with one or more substituents. In certain embodiments, the heterocyclyl group is unsubstituted 3-10 membered heterocyclyl. In certain embodiments, the heterocyclyl group is substituted 3-10 membered heterocyclyl.

In some embodiments, a heterocyclyl group is a 5-10 membered non-aromatic ring system having ring carbon atoms and 1-4 ring heteroatoms, wherein each heteroatom is independently selected from nitrogen, oxygen, sulfur, boron, phosphorus, and silicon (“5-10 membered heterocyclyl”). In some embodiments, a heterocyclyl group is a 5-8 membered non-aromatic ring system having ring carbon atoms and 1-4 ring heteroatoms, wherein each heteroatom is independently selected from nitrogen, oxygen, and sulfur (“5-8 membered heterocyclyl”). In some embodiments, a heterocyclyl group is a 5-6 membered non-aromatic ring system having ring carbon atoms and 1-4 ring heteroatoms, wherein each heteroatom is independently selected from nitrogen, oxygen, and sulfur (“5-6 membered heterocyclyl”). In some embodiments, the 5-6 membered heterocyclyl has 1-3 ring heteroatoms selected from nitrogen, oxygen, and sulfur. In some embodiments, the 5-6 membered heterocyclyl has 1-2 ring heteroatoms selected from nitrogen, oxygen, and sulfur. In some embodiments, the 5-6 membered heterocyclyl has one ring heteroatom selected from nitrogen, oxygen, and sulfur.

Exemplary 3-membered heterocyclyl groups containing one heteroatom include, without limitation, azirdinyl, oxiranyl, thiorenyl. Exemplary 4-membered heterocyclyl groups containing one heteroatom include, without limitation, azetidinyl, oxetanyl and thietanyl. Exemplary 5-membered heterocyclyl groups containing one heteroatom include, without limitation, tetrahydrofuranyl, dihydrofuranyl, tetrahydrothiophenyl, dihydrothiophenyl, pyrrolidinyl, dihydropyrrolyl and pyrrolyl-2,5-dione. Exemplary 5-membered heterocyclyl groups containing two heteroatoms include, without limitation, dioxolanyl, oxasulfuranyl, disulfuranyl, and oxazolidin-2-one. Exemplary 5-membered heterocyclyl groups containing three heteroatoms include, without limitation, triazolinyl, oxadiazolinyl, and thiadiazolinyl.

Exemplary 6-membered heterocyclyl groups containing one heteroatom include, without limitation, piperidinyl, tetrahydropyranyl, dihydropyridinyl, and thianyl. Exemplary 6-membered heterocyclyl groups containing two heteroatoms include, without limitation, piperazinyl, morpholinyl, dithianyl, dioxanyl. Exemplary 6-membered heterocyclyl groups containing two heteroatoms include, without limitation, triazinanyl. Exemplary 7-membered heterocyclyl groups containing one heteroatom include, without limitation, azepanyl, oxepanyl and thiepanyl. Exemplary 8-membered heterocyclyl groups containing one heteroatom include, without limitation, azocanyl, oxecanyl and thiocanyl. Exemplary 5-membered heterocyclyl groups fused to a C₆ aryl ring (also referred to herein as a 5,6-bicyclic heterocyclic ring) include, without limitation, indolinyl, isoindolinyl, dihydrobenzofuranyl, dihydrobenzothienyl, benzoxazolinonyl, and the like. Exemplary 6-membered heterocyclyl groups fused to an aryl ring (also referred to herein as a 6,6-bicyclic heterocyclic ring) include, without limitation, tetrahydroquinolinyl, tetrahydroisoquinolinyl, and the like.

As used herein, “arylalkyl” refers to an (aryl)alkyl-radical wherein aryl and alkyl moieties are as disclosed herein.

As used herein, “cycloalkylalkyl” as used herein refers to a -(cycloalkyl)-alkyl radical where cycloalkyl and alkyl are as defined herein.

As used herein, “heteroarylalkyl” refers to refers to an (heteroaryl)alkyl-radical wherein the heteroaryl and alkyl moieties are as disclosed herein.

As used herein, “heterocycloalkyl” refers to an (heterocyclyl)alkyl-radical wherein the heteroaryl and alkyl moieties are as disclosed herein.

“Cyano” refers to the radical —CN.

As used herein, “halo” or “halogen,” independently or as part of another substituent, mean, unless otherwise stated, a fluorine (F), chlorine (Cl), bromine (Br), or iodine (I) atom.

As used herein, “haloalkyl” can include alkyl structures that are substituted with one or more halo groups or with combinations thereof. For example, the terms “fluoroalkyl” includes haloalkyl groups in which the halo is fluorine (e.g., —C₁-C₆ alkyl-CF₃, —C₁-C₆ alkyl-C₂F). Non-limiting examples of haloalkyl include trifluoroethyl, trifluoropropyl, trifluoromethyl, fluoromethyl, diflurormethyl, and fluroisopropyl.

As used herein, “hydroxy” refers to the radical —OH.

As used herein, “nitro” refers to —NO₂.

As used herein, “keto” refers to —C═O.

Two or more substituents may optionally be joined to form aryl, heteroaryl, cycloalkyl, or heterocyclyl groups. Such so-called ring-forming substituents are typically, though not necessarily, found attached to a cyclic base structure. In one embodiment, the ring-forming substituents are attached to adjacent members of the base structure. For example, two ring-forming substituents attached to adjacent members of a cyclic base structure create a fused ring structure. In another embodiment, the ring-forming substituents are attached to a single member of the base structure. For example, two ring-forming substituents attached to a single member of a cyclic base structure create a spirocyclic structure. In yet another embodiment, the ring-forming substituents are attached to non-adjacent members of the base structure.

Compounds described herein can comprise one or more asymmetric centers, and thus can exist in various isomeric forms, e.g., enantiomers and/or diastereomers. For example, the compounds described herein can be in the form of an individual enantiomer, diastereomer or geometric isomer, or can be in the form of a mixture of stereoisomers, including racemic mixtures and mixtures enriched in one or more stereoisomer. Isomers can be isolated from mixtures by methods known to those skilled in the art, including chiral high pressure liquid chromatography (HPLC) and the formation and crystallization of chiral salts; or preferred isomers can be prepared by asymmetric syntheses. See, for example, Jacques et al., Enantiomers, Racemates and Resolutions (Wiley Interscience, New York, 1981); Wilen et al., Tetrahedron 33:2725 (1977); Eliel, Stereochemistry of Carbon Compounds (McGraw-Hill, N Y, 1962); and Wilen, Tables of Resolving Agents and Optical Resolutions p. 268 (E. L. Eliel, Ed., Univ. of Notre Dame Press, Notre Dame, Ind. 1972). The invention additionally encompasses compounds described herein as individual isomers substantially free of other isomers, and alternatively, as mixtures of various isomers.

As used herein, a pure enantiomeric compound is substantially free from other enantiomers or stereoisomers of the compound (i.e., in enantiomeric excess). In other words, an “S” form of the compound is substantially free from the “R” form of the compound and is, thus, in enantiomeric excess of the “R” form. The term “enantiomerically pure” or “pure enantiomer” denotes that the compound comprises more than 75% by weight, more than 80% by weight, more than 85% by weight, more than 90% by weight, more than 91% by weight, more than 92% by weight, more than 93% by weight, more than 94% by weight, more than 95% by weight, more than 96% by weight, more than 97% by weight, more than 98% by weight, more than 99% by weight, more than 99.5% by weight, or more than 99.9% by weight, of the enantiomer. In certain embodiments, the weights are based upon total weight of all enantiomers or stereoisomers of the compound.

In the compositions provided herein, an enantiomerically pure compound can be present with other active or inactive ingredients. For example, a pharmaceutical composition comprising enantiomerically pure R-compound can comprise, for example, about 90% excipient and about 10% enantiomerically pure R-compound. In certain embodiments, the enantiomerically pure R-compound in such compositions can, for example, comprise, at least about 95% by weight R-compound and at most about 5% by weight S-compound, by total weight of the compound. For example, a pharmaceutical composition comprising enantiomerically pure S-compound can comprise, for example, about 90% excipient and about 10% enantiomerically pure S-compound.

In certain embodiments, the enantiomerically pure S-compound in such compositions can, for example, comprise, at least about 95% by weight S-compound and at most about 5% by weight R-compound, by total weight of the compound. In certain embodiments, the active ingredient can be formulated with little or no excipient or carrier.

Compound described herein may also comprise one or more isotopic substitutions. For example, H may be in any isotopic form, including ¹H, ²H (D or deuterium), and ³H (T or tritium); C may be in any isotopic form, including ¹²C, ¹³C, and ¹⁴C; O may be in any isotopic form, including 160 and ¹⁸O; and the like.

The term “pharmaceutically acceptable salt” is meant to include salts of the active compounds that are prepared with relatively nontoxic acids or bases, depending on the particular substituents found on the compounds described herein. When compounds of the present invention contain relatively acidic functionalities, base addition salts can be obtained by contacting the neutral form of such compounds with a sufficient amount of the desired base, either neat or in a suitable inert solvent. Examples of pharmaceutically acceptable base addition salts include sodium, potassium, calcium, ammonium, organic amino, or magnesium salt, or a similar salt. When compounds of the present invention contain relatively basic functionalities, acid addition salts can be obtained by contacting the neutral form of such compounds with a sufficient amount of the desired acid, either neat or in a suitable inert solvent. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from organic acids like acetic, propionic, isobutyric, maleic, malonic, benzoic, succinic, suberic, fumaric, lactic, mandelic, phthalic, benzenesulfonic, p-tolylsulfonic, citric, tartaric, methanesulfonic, and the like. Also included are salts of amino acids such as arginate and the like, and salts of organic acids like glucuronic or galactunoric acids and the like (see, e.g., Berge et al, Journal of Pharmaceutical Science 66: 1-19 (1977)). Certain specific compounds of the present invention contain both basic and acidic functionalities that allow the compounds to be converted into either base or acid addition salts. These salts may be prepared by methods known to those skilled in the art. Other pharmaceutically acceptable carriers known to those of skill in the art are suitable for the present invention.

Many of the terms given above may be used repeatedly in the definition of a formula or group and in each case have one of the meanings given above, independently of one another.

As used herein, the term “substituted” or “substituted with” is contemplated to include all permissible substituents of organic compounds. In a broad aspect, the permissible substituents include acyclic and cyclic, branched and unbranched, carbocyclic and heterocyclic, aromatic and nonaromatic substituents of organic compounds (e.g., alkyl, alkenyl, alkynyl, cycloalkyl, heterocycloalkyl, aryl, or heteroaryl, any of which may itself be further substituted), as well as halogen, carbonyl (e.g., aldehyde, ketone, ester, carboxyl, or formyl), thiocarbonyl (e.g., thioester, thiocarboxylate, or thioformate), amino, —N(R^{b})(R^{c}), wherein each R^{b} and R^{C} is independently H or C₁-C₆ alkyl, cyano, nitro, —SO₂N(R^{b})(R^{c}), —SOR^{d}, and S(O)₂R^{d}, wherein each R^{b}, R^{C}, and R^{d} is independently H or C₁-C₆ alkyl. Illustrative substituents include, for example, those described herein above. The permissible substituents can be one or more and the same or different for appropriate organic compounds. For purposes of this invention, the heteroatoms such as nitrogen may have hydrogen substituents and/or any permissible substituents of organic compounds described herein which satisfy the valences of the heteroatoms. This invention is not intended to be limited in any manner by the permissible substituents of organic compounds.

It will be understood that “substitution” or “substituted with” includes the implicit proviso that such substitution is in accordance with permitted valence of the substituted atom and the substituent, and that the substitution results in a stable compound, e.g., which does not spontaneously undergo transformation such as by rearrangement, cyclization, elimination, etc.

Contemplated equivalents of the compounds described above include compounds which otherwise correspond thereto, and which have the same general properties thereof (e.g., the ability to inhibit the formation of TDP-43 inclusions), wherein one or more simple variations of substituents are made which do not adversely affect the efficacy of the compound. In general, the compounds of the present invention may be prepared by the methods illustrated in the general reaction schemes as, for example, described below, or by modifications thereof, using readily available starting materials, reagents and conventional synthesis procedures. In these reactions, it is also possible to make use of variants which are in themselves known, but are not mentioned here.

For purposes of this invention, the chemical elements are identified in accordance with the Periodic Table of the Elements, CAS version, Handbook of Chemistry and Physics, 67th Ed., 1986-87, inside cover. Also for purposes of this invention, the term “hydrocarbon” is contemplated to include all permissible compounds having at least one hydrogen and one carbon atom. In a broad aspect, the permissible hydrocarbons include acyclic and cyclic, branched and unbranched, carbocyclic and heterocyclic, aromatic and nonaromatic organic compounds which can be substituted or unsubstituted.

### Pharmaceutical Compositions and Routes of Administration

Pharmaceutical compositions containing compounds described herein such as a compound of Formula (I) or pharmaceutically acceptable salt thereof can be used to treat or ameliorate a disorder described herein, for example, a neurodegenerative disease, a cancer, an ophthalmological disease (e.g., a retinal disease), or a viral infection.

The amount and concentration of compounds of Formula (I) in the pharmaceutical compositions, as well as the quantity of the pharmaceutical composition administered to a subject, can be selected based on clinically relevant factors, such as medically relevant characteristics of the subject (e.g., age, weight, gender, other medical conditions, and the like), the solubility of compounds in the pharmaceutical compositions, the potency and activity of the compounds, and the manner of administration of the pharmaceutical compositions. For further information on Routes of Administration and Dosage Regimes the reader is referred to Chapter 25.3 in Volume 5 of Comprehensive Medicinal Chemistry (Corwin Hansch; Chairman of Editorial Board), Pergamon Press 1990.

While it is possible for a compound of the present invention to be administered alone, it is preferable to administer the compound as a pharmaceutical formulation (composition), where the compound is combined with one or more pharmaceutically acceptable diluents, excipients or carriers. The compounds according to the invention may be formulated for administration in any convenient way for use in human or veterinary medicine. In certain embodiments, the compound included in the pharmaceutical preparation may be active itself, or may be a prodrug, e.g., capable of being converted to an active compound in a physiological setting. Regardless of the route of administration selected, the compounds of the present invention, which may be used in a suitable hydrated form, and/or the pharmaceutical compositions of the present invention, are formulated into pharmaceutically acceptable dosage forms such as described below or by other conventional methods known to those of skill in the art.

Thus, another aspect of the present invention provides pharmaceutically acceptable compositions comprising a therapeutically effective amount of one or more of the compounds described above, formulated together with one or more pharmaceutically acceptable carriers (additives) and/or diluents. As described in detail below, the pharmaceutical compositions of the present invention may be specially formulated for administration in solid or liquid form, including those adapted for the following: (1) oral administration, for example, drenches (aqueous or non-aqueous solutions or suspensions), lozenges, dragees, capsules, pills, tablets (e.g., those targeted for buccal, sublingual, and systemic absorption), boluses, powders, granules, pastes for application to the tongue; (2) parenteral administration, for example, by subcutaneous, intramuscular, intravenous or epidural injection as, for example, a sterile solution or suspension, or sustained-release formulation; (3) topical application, for example, as a cream, ointment, or a controlled-release patch or spray applied to the skin; (4) intravaginally or intrarectally, for example, as a pessary, cream or foam; (5) sublingually; (6) ocularly; (7) transdermally; (8) transmucosally; (9) nasally; or (10) intrathecally. Additionally, compounds can be implanted into a patient or injected using a drug delivery system. See, for example, Urquhart, et al., (1994) Ann Rev Pharmacol Toxicol 24:199-236; Lewis, ed. “Controlled Release of Pesticides and Pharmaceuticals” (Plenum Press, New York, 1981); U.S. Pat. No. 3,773,919; and U.S. Pat. No. 35 3,270,960.

The phrase “therapeutically effective amount” as used herein means that amount of a compound, material, or composition comprising a compound of the present invention which is effective for producing some desired therapeutic effect, e.g., by inhibiting TDP-43 inclusions, in at least a sub-population of cells in an animal and thereby blocking the biological consequences of that function in the treated cells, at a reasonable benefit/risk ratio applicable to any medical treatment.

The phrases “systemic administration,” “administered systemically,” “peripheral administration” and “administered peripherally” as used herein mean the administration of a compound, drug or other material other than directly into the central nervous system, such that it enters the patient's system and, thus, is subject to metabolism and other like processes, for example, subcutaneous administration.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically acceptable carrier” as used herein means a pharmaceutically acceptable material, composition or vehicle, such as a liquid or solid filler, diluent, excipient, solvent or encapsulating material, involved in carrying or transporting the subject antagonists from one organ, or portion of the body, to another organ, or portion of the body. Each carrier must be “acceptable” in the sense of being compatible with the other ingredients of the formulation and not injurious to the patient. Some examples of materials which can serve as pharmaceutically acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; (21) cyclodextrins such as Captisol®; and (22) other non-toxic compatible substances employed in pharmaceutical formulations.

As set out above, certain embodiments of the present compounds may contain a basic functional group, such as amino or alkylamino, and are, thus, capable of forming pharmaceutically acceptable salts with pharmaceutically acceptable acids. The term “pharmaceutically acceptable salts” in this respect, refers to the relatively non-toxic, inorganic and organic acid addition salts of compounds of the present invention. These salts can be prepared in situ during the final isolation and purification of the compounds of the invention, or by separately reacting a purified compound of the invention in its free base form with a suitable organic or inorganic acid, and isolating the salt thus formed. Representative salts include the hydrobromide, hydrochloride, sulfate, bisulfate, phosphate, nitrate, acetate, valerate, oleate, palmitate, stearate, laurate, benzoate, lactate, phosphate, tosylate, citrate, maleate, fumarate, succinate, tartrate, napthylate, mesylate, glucoheptonate, lactobionate, and laurylsulphonate salts and the like (see, for example, Berge et al. (1977) “Pharmaceutical Salts”, J Pharm Sci 66:1-19).

The pharmaceutically acceptable salts of the subject compounds include the conventional nontoxic salts or quaternary ammonium salts of the compounds, e.g., from non-toxic organic or inorganic acids. For example, such conventional nontoxic salts include those derived from inorganic acids such as hydrochloride, hydrobromic, sulfuric, sulfamic, phosphoric, nitric, and the like; and the salts prepared from organic acids such as acetic, propionic, succinic, glycolic, stearic, lactic, malic, tartaric, citric, ascorbic, palmitic, maleic, hydroxymaleic, phenylacetic, glutamic, benzoic, salicyclic, sulfanilic, 2-acetoxybenzoic, fumaric, toluenesulfonic, methanesulfonic, ethane disulfonic, oxalic, isothionic, and the like.

In other cases, the compounds of the present invention may contain one or more acidic functional groups and, thus, are capable of forming pharmaceutically acceptable salts with pharmaceutically acceptable bases. The term “pharmaceutically acceptable salts” in these instances refers to the relatively non-toxic, inorganic and organic base addition salts of compounds of the present invention. These salts can likewise be prepared in situ during the final isolation and purification of the compounds, or by separately reacting the purified compound in its free acid form with a suitable base, such as the hydroxide, carbonate or bicarbonate of a pharmaceutically acceptable metal cation, with ammonia, or with a pharmaceutically acceptable organic primary, secondary or tertiary amine. Representative alkali or alkaline earth salts include the lithium, sodium, potassium, calcium, magnesium, and aluminum salts and the like. Representative organic amines useful for the formation of base addition salts include ethylamine, diethylamine, ethylenediamine, ethanolamine, diethanolamine, piperazine and the like (see, for example, Berge et al., supra).

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically acceptable antioxidants include: (1) water soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal chelating agents, such as citric acid, ethylenediamine tetraacetic acid (EDTA), sorbitol, tartaric acid, phosphoric acid, and the like.

Formulations of the present invention include those suitable for oral, nasal, topical (including buccal and sublingual), rectal, vaginal and/or parenteral administration. The formulations may conveniently be presented in unit dosage form and may be prepared by any methods well known in the art of pharmacy. The amount of active ingredient which can be combined with a carrier material to produce a single dosage form will vary depending upon the host being treated, the particular mode of administration. The amount of active ingredient that can be combined with a carrier material to produce a single dosage form will generally be that amount of the compound which produces a therapeutic effect. Generally, out of one hundred percent, this amount will range from about 1 percent to about ninety-nine percent of active ingredient, preferably from about 5 percent to about 70 percent, most preferably from about 10 percent to about 30 percent.

Methods of preparing these formulations or compositions include the step of bringing into association a compound of the present invention with the carrier and, optionally, one or more accessory ingredients. In general, the formulations are prepared by uniformly and intimately bringing into association a compound of the present invention with liquid carriers, or finely divided solid carriers, or both, and then, if necessary, shaping the product.

Formulations of the invention suitable for oral administration may be in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a compound of the present invention as an active ingredient. A compound of the present invention may also be administered as a bolus, electuary or paste.

In solid dosage forms of the invention for oral administration (capsules, tablets, pills, dragees, powders, granules and the like), the active ingredient is mixed with one or more pharmaceutically acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds; (7) wetting agents, such as, for example, cetyl alcohol and glycerol monostearate; (8) absorbents, such as kaolin and bentonite clay; (9) lubricants, such a talc, calcium stearate, magnesium stearate, solid polyethylene glycols, sodium lauryl sulfate, and mixtures thereof; and (10) coloring agents. In the case of capsules, tablets and pills, the pharmaceutical compositions may also comprise buffering agents. Solid compositions of a similar type may also be employed as fillers in soft and hard-filled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered compound moistened with an inert liquid diluent.

The tablets, and other solid dosage forms of the pharmaceutical compositions of the present invention, such as dragees, capsules, pills and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxypropylmethyl cellulose in varying proportions to provide the desired release profile, other polymer matrices, liposomes and/or microspheres. They may be sterilized by, for example, filtration through a bacteria-retaining filter, or by incorporating sterilizing agents in the form of sterile solid compositions that can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner. Examples of embedding compositions that can be used include polymeric substances and waxes. The active ingredient can also be in micro-encapsulated form, if appropriate, with one or more of the above-described excipients.

Liquid dosage forms for oral administration of the compounds of the invention include pharmaceutically acceptable emulsions, microemulsions, solutions, suspensions, syrups and elixirs. In addition to the active ingredient, the liquid dosage forms may contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

Besides inert diluents, the oral compositions can also include adjuvants such as wetting agents, emulsifying and suspending agents, sweetening, flavoring, coloring, perfuming and preservative agents.

Suspensions, in addition to the active compounds, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Formulations of the pharmaceutical compositions of the invention for rectal, vaginal, or urethral administration may be presented as a suppository, which may be prepared by mixing one or more compounds of the invention with one or more suitable nonirritating excipients or carriers comprising, for example, cocoa butter, polyethylene glycol, a suppository wax or a salicylate, and which is solid at room temperature, but liquid at body temperature and, therefore, will melt in the rectum or vaginal cavity and release the active compound.

Alternatively or additionally, compositions can be formulated for delivery via a catheter, stent, wire, or other intraluminal device. Delivery via such devices may be especially useful for delivery to the heart, lung, bladder, urethra, ureter, rectum, or intestine. Furthermore, compositions can be formulated for delivery via a dialysis port.

Ophthalmic formulations, eye ointments, powders, solutions and the like, are also contemplated as being within the scope of this invention.

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal and intrasternal injection and infusion. Pharmaceutical compositions of this invention suitable for parenteral administration comprise one or more compounds of the invention in combination with one or more pharmaceutically acceptable sterile isotonic aqueous or nonaqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents.

Examples of suitable aqueous and nonaqueous carriers that may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of the action of microorganisms may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents that delay absorption such as aluminum monostearate and gelatin.

In some cases, in order to prolong the effect of a drug, it is desirable to slow the absorption of the drug from subcutaneous or intramuscular injection. This may be accomplished by the use of a liquid suspension of crystalline or amorphous material having poor water solubility. The rate of absorption of the drug then depends upon its rate of dissolution, which, in turn, may depend upon crystal size and crystalline form. Alternatively, delayed absorption of a parenterally administered drug form is accomplished by dissolving or suspending the drug in an oil vehicle.

Injectable depot forms are made by forming microencapsule matrices of the subject compounds in biodegradable polymers such as polylactide-polyglycolide. Depending on the ratio of drug to polymer, and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions that are compatible with body tissue.

When the compounds of the present invention are administered as pharmaceuticals, to humans and animals, they can be given per se or as a pharmaceutical composition containing, for example, 0.1 to 99.5% (more preferably, 0.5 to 90%) of active ingredient in combination with a pharmaceutically acceptable carrier.

The addition of the active compound of the invention to animal feed is preferably accomplished by preparing an appropriate feed premix containing the active compound in an effective amount and incorporating the premix into the complete ration. Alternatively, an intermediate concentrate or feed supplement containing the active ingredient can be blended into the feed. The way in which such feed premixes and complete rations can be prepared and administered are described in reference books (such as “Applied Animal Nutrition”, W.H.

Freedman and CO., San Francisco, U.S.A., 1969 or “Livestock Feeds and Feeding” O and B books, Corvallis, Ore., U.S.A., 1977).

Methods of introduction may also be provided by rechargeable or biodegradable devices. Various slow release polymeric devices have been developed and tested in vivo in recent years for the controlled delivery of drugs, including proteinacious biopharmaceuticals. A variety of biocompatible polymers (including hydrogels), including both biodegradable and non-degradable polymers, can be used to form an implant for the sustained release of a compound at a particular target site.

Preferably, the subject is a mammal. The mammal can be a human, non-human primate, mouse, rat, dog, cat, horse, or cow, but are not limited to these examples. Mammals other than humans can be advantageously used as subjects that represent animal models of disorders associated with neurodegenerative disease or disorder, cancer, or viral infections.

In addition, the methods described herein can be used to treat domesticated animals and/or pets. A subject can be male or female. A subject can be one who has been previously diagnosed with or identified as suffering from or having a neurodegenerative disease or disorder, a disease or disorder associated with cancer, a disease or disorder associated with viral infection, or one or more complications related to such diseases or disorders but need not have already undergone treatment.

### Dosages

Actual dosage levels of the active ingredients in the pharmaceutical compositions of this invention may be varied so as to obtain an amount of the active ingredient that is effective to achieve the desired therapeutic response for a particular patient, composition, and mode of administration, without being toxic to the patient.

The selected dosage level will depend upon a variety of factors including the activity of the particular compound of the present invention employed, or the ester, salt or amide thereof, the route of administration, the time of administration, the rate of excretion of the particular compound being employed, the duration of the treatment, other drugs, compounds and/or materials used in combination with the particular compound employed, the age, sex, weight, condition, general health and prior medical history of the patient being treated, and like factors well known in the medical arts.

A physician or veterinarian having ordinary skill in the art can readily determine and prescribe the effective amount of the pharmaceutical composition required. For example, the physician or veterinarian could start doses of the compounds of the invention employed in the pharmaceutical composition at levels lower than that required in order to achieve the desired therapeutic effect and gradually increase the dosage until the desired effect is achieved.

The compound and the pharmaceutically active agent can be administrated to the subject in the same pharmaceutical composition or in different pharmaceutical compositions (at the same time or at different times). When administrated at different times, the compound and the pharmaceutically active agent can be administered within 5 minutes, 10 minutes, 20 minutes, 60 minutes, 2 hours, 3 hours, 4, hours, 8 hours, 12 hours, 24 hours of administration of the other agent. When the inhibitor and the pharmaceutically active agent are administered in different pharmaceutical compositions, routes of administration can be different.

The amount of compound that can be combined with a carrier material to produce a single dosage form will generally be that amount of the inhibitor that produces a therapeutic effect. Generally out of one hundred percent, this amount will range from about 0.1% to 99% of inhibitor, preferably from about 5% to about 70%, most preferably from 10% to about 30%.

Toxicity and therapeutic efficacy can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Compositions that exhibit large therapeutic indices are preferred.

The data obtained from the cell culture assays and animal studies can be used in formulating a range of dosage for use in humans. The dosage of such compounds lies preferably within a range of circulating concentrations that include the ED₅₀ with little or no toxicity. The dosage may vary within this range depending upon the dosage form employed and the route of administration utilized.

The therapeutically effective dose can be estimated initially from cell culture assays. A dose may be formulated in animal models to achieve a circulating plasma concentration range that includes the IC₅₀ (i.e., the concentration of the therapeutic which achieves a half-maximal inhibition of symptoms) as determined in cell culture. Levels in plasma may be measured, for example, by high performance liquid chromatography. The effects of any particular dosage can be monitored by a suitable bioassay.

The dosage may be determined by a physician and adjusted, as necessary, to suit observed effects of the treatment. Generally, the compositions are administered so that the compound of Formula (I) is given at a dose from 1 ng/kg to 200 mg/kg, 10 ng/kg to 100 mg/kg, 10 ng/kg to 50 mg/kg, 100 ng/kg to 20 mg/kg, 100 ng/kg to 10 mg/kg, 100 ng/kg to 1 mg/kg, 1 μg/kg to 100 mg/kg, 1 μg/kg to 50 mg/kg, 1 μg/kg to 20 mg/kg, 1 μg/kg to 10 mg/kg, 1 μg/kg to 1 mg/kg, 10 μg/kg to 10 mg/kg, 10 μg/kg to 50 mg/kg, 10 mg/kg to 20 mg/kg, 10 μg/kg to 10 mg/kg, 10 μg/kg to 1 mg/kg, 100 μg/kg to 50 mg/kg, 100 μg/kg to 20 mg/kg, 1 mg/kg to 100 mg/kg, 1 mg/kg to 50 mg/kg, 1 mg/kg to 20 mg/kg, 1 mg/kg to 10 mg/kg, 1 μg/kg to 10 mg/kg, 10 mg/kg to 100 mg/kg, 10 mg/kg to 50 mg/kg, 10 mg/kg to 20 mg/kg, or 50 mg/kg to 100 mg/kg. It is to be understood that ranges given here include all intermediate ranges, e.g., the range 1 mg/kg to 10 mg/kg includes 1 mg/kg to 2 mg/kg, 1 mg/kg to 3 mg/kg, 1 mg/kg to 4 mg/kg, 1 mg/kg to 5 mg/kg, 1 mg/kg to 6 mg/kg, 1 mg/kg to 7 mg/kg, 1 mg/kg to 8 mg/kg, 1 mg/kg to 9 mg/kg, 2 mg/kg to 10 mg/kg, 3 mg/kg to 10 mg/kg, 4 mg/kg to 10 mg/kg, 5 mg/kg to 10 mg/kg, 6 mg/kg to 10 mg/kg, 7 mg/kg to 10 mg/kg, 8 mg/kg to 10 mg/kg, 9 mg/kg to 10 mg/kg, and the like. It is to be further understood that the ranges intermediate to the given above are also within the scope of this invention, for example, in the range 1 mg/kg to 10 mg/kg, dose ranges such as 2 mg/kg to 8 mg/kg, 3 mg/kg to 7 mg/kg, 4 mg/kg to 6 mg/kg, and the like.

With respect to duration and frequency of treatment, it is typical for skilled clinicians to monitor subjects in order to determine when the treatment is providing therapeutic benefit, and to determine whether to increase or decrease dosage, increase or decrease administration frequency, discontinue treatment, resume treatment or make other alteration to treatment regimen. The dosing schedule can vary from once a week to daily depending on a number of clinical factors, such as the subject's sensitivity to the drugs. The desired dose can be administered at one time or divided into subdoses, e.g., 2-4 subdoses and administered over a period of time, e.g., at appropriate intervals through the day or other appropriate schedule. Such sub-doses can be administered as unit dosage forms. In some embodiments, administration is chronic, e.g., one or more doses daily over a period of weeks or months. Examples of dosing schedules are administration daily, twice daily, three times daily or four or more times daily over a period of 1 week, 2 weeks, 3 weeks, 4 weeks, 1 month, 2 months, 3 months, 4 months, 5 months, or 6 months or more.

The present invention contemplates formulation of the subject compounds in any of the aforementioned pharmaceutical compositions and preparations. Furthermore, the present invention contemplates administration via any of the foregoing routes of administration. One of skill in the art can select the appropriate formulation and route of administration based on the condition being treated and the overall health, age, and size of the patient being treated.

### EXAMPLES

Examples are provided below to facilitate a more complete understanding of the invention. The following examples illustrate exemplary modes of making and practicing the invention. However, the scope of the invention is not limited to specific embodiments disclosed in these Examples, which are for purposes of illustration only, since alternative methods can be utilized to obtain similar results.

### General.

All oxygen and/or moisture sensitive reactions were carried out under N₂ atmosphere in glassware that was flame-dried under vacuum (0.5 mmHg) and purged with N₂ prior to use. All reagents and solvents were purchased from commercial vendors and used as received, or synthesized according to the footnoted references. NMR spectra were recorded on a Bruker 400 (400 MHz ¹H, 75 MHz ¹³C) or Varian (400 MHz ¹H, 75 MHz ¹³C) spectrometer. Proton and carbon chemical shifts are reported in ppm (δ) referenced to the NMR solvent. Data are reported as follows: chemical shifts, multiplicity (br=broad, s=singlet, t=triplet, q=quartet, m=multiplet; coupling constant (s) in Hz). Unless otherwise indicated NMR data were collected at 25° C. Flash chromatography was performed using 100-200 mesh Silica Gel. Liquid Chromatography/Mass Spectrometry (LCMS) was performed on Agilent 1200HPLC and 6110MS. Analytical thin layer chromatography (TLC) was performed on 0.2 mm silica gel plates. Visualization was accomplished with UV light and aqueous potassium permanganate (KMnO₄) stain followed by heating.

TABLE 1
Abbreviations

| | |
|---|---|
| ACN | acetonitrile |
| Bn | benzyl |
| Boc | t-butoxycarbonyl |
| t-BuXphos | 2-di-t-butylphosphino-2′,4′,6′-triisopropylbiphenyl |
| t-BuOK | potassium tert-butoxide |
| DCM | dichloromethane |
| DIBALH | diisobutylaluminum hydride |
| DIEA | diisopropylethylamine |
| DMAP | N,N-4-dimethylaminopyridine |
| DMF | N,N-dimethylformamide |
| DMSO | dimethyl sulfoxide |
| EDCl | 1-ethyl-3-(3-dimethylaminopropyl)carbodiimide |
| | hydrochloride |
| EtOAc | ethyl acetate |
| HATU | O-(7-azabenzotriazol-1-yl)-N,N,N′,N′- |
| | tetramethyluronium hexafluorophosphate |
| HOAc | acetic acid |
| HOBT | N-hydroxybenztriazole |
| Hrs | hours |
| LCMS | liquid chromatography-mass specrtum |
| Me | methyl |
| MeOH | methanol |
| MsCl | methanesulfonyl chloride |
| Pd₂(dba)₃ | tris(dibenzylideneacetone)dipalladium |
| Ph | phenyl |
| PMB | p-methoxybenzyl |
| PTSA | p-toluenesulfonic acid |
| Py or pyr | pyridine |
| TBME | tert-butylmethyl ether |
| TEA | triethylamine |
| TFA | trifluoroacetic acid |
| THF | tetrahydrofuran |
| TLC | Thin layer chromatography |

### Example 1. Synthesis of N-(3,5-dimethoxyphenyl)-3-(1-(4-fluoro-3-methoxybenzyl)-piperidin-3-yl)propanamide (Compound 100)

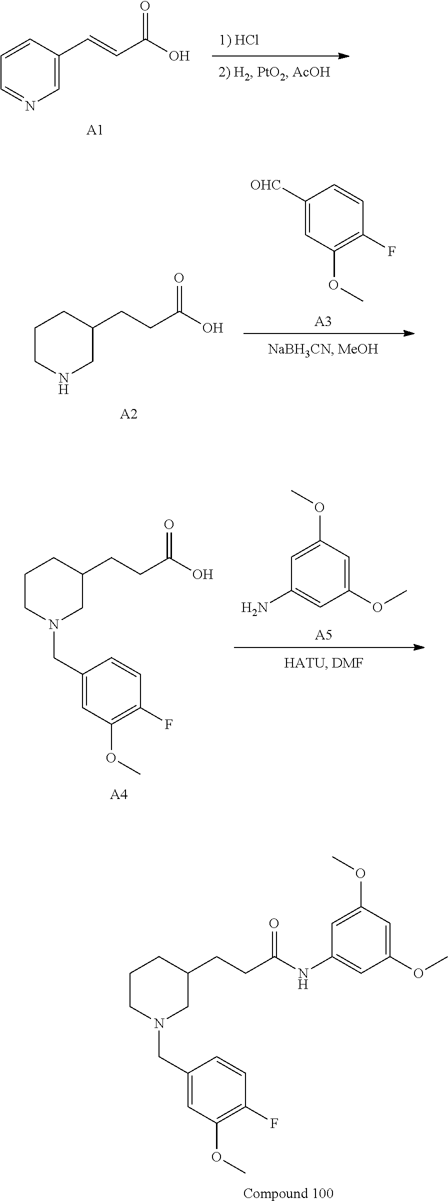

### Step 1: Synthesis of A2

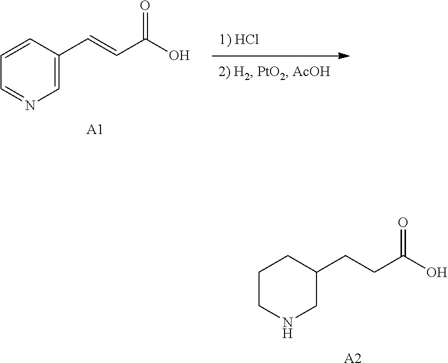 [SMILES: O=C(O)/C=C/c1cccnc1 ; "A1" ; O=C(O)CCC1CCCNC1 ; "A2"]

To a solution of A1 (2 g, 13 mmol, 1.00 eq) in dioxane (50 mL) was added cone. HCl (2 mL). The mixture was stirred at 25° C. for 30 min and the solvent was evaporated under reduced pressure. The residue was dissolved into AcOH (50 mL) and PtO₂ (487 mg, 2.2 mmol, 0.15 eq) was added. The suspension was degassed under vacuum and purged with H₂ several times. The mixture was stirred under H₂ (50 psi) at 25° C. for 12 hrs, at which point LCMS showed the reaction was complete. The mixture was diluted with water (100 mL) and filtered, and the catalyst washed with water, keeping the catalyst wet at all times. The filtrate was concentrated under reduced pressure to afford A2 (2 g, 13 mmol, 95.0% yield) as a white solid. ¹H NMR: (CDCl₃ 400 MHz) δ 3.29 (d, J=11.8 Hz, 2H) 2.83 (t, J=12.2 Hz, 1H) 2.60 (t, J=11.8 Hz, 1H) 2.38 (br. s., 2H) 1.86 (d, J=11.8 Hz, 2H) 1.43-1.76 (m, 4H) 1.14 (q, J=11.4 Hz, 1H).

### Step 2: Synthesis of A4

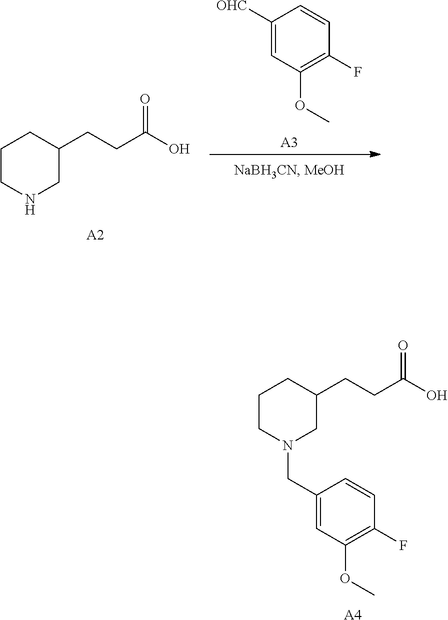

A solution of A2 (2 g, 13 mmol, 1 eq) and A3 (3 g, 19 mmol, 1.5 eq) in MeOH (50 mL) was stirred at 25° C. for 1 hr, followed by addition of NaBH₃CN (1.2 g, 19 mmol, 1.5 eq). The mixture was stirred at 25° C. for 12 hrs, at which point LCMS analysis showed the reaction was complete. The mixture was diluted with water (100 mL) and concentrated under vacuum, and a solution of saturated NaHCO₃ (50 mL) was added into the mixture (pH=9) and extracted with ethyl acetate (100 mL*2). The aqueous pH was adjusted to 6 with HCl (1 M, 5 mL) and extracted with ethyl acetate (100 mL*3). The combined organic phase was washed with brine (100 mL*2), dried with anhydrous Na₂SO₄, filtered and concentrated under vacuum to give A4 (1.00 g, 3.39 mmol, 26.6% yield) as a white solid. ¹H NMR: (CDCl₃ 400 MHz) δ 7.27 (dd, J=8.0, 1.8 Hz, 1H) 7.20 (dd, J=11.2, 8.2 Hz, 1H) 7.06 (ddd, J=8.0, 4.0, 2.0 Hz, 1H) 4.22-4.35 (m, 2H) 3.94 (s, 3H) 3.43 (d, J=11.0 Hz, 2H) 2.92 (t, J=11.4 Hz, 1H) 2.71 (t, J=12.0 Hz, 1H) 2.30-2.44 (m, 2H) 1.91-2.05 (m, 2H) 1.69-1.89 (m, 2H) 1.52-1.67 (m, 2H) 1.12-1.28 (m, 1H).

### Step 3: Synthesis of N-(3,5-dimethoxyphenyl)-3-(1-(4-fluoro-3-methoxybenzyl)piperidin-3-yl)propanamide (Compound 100)

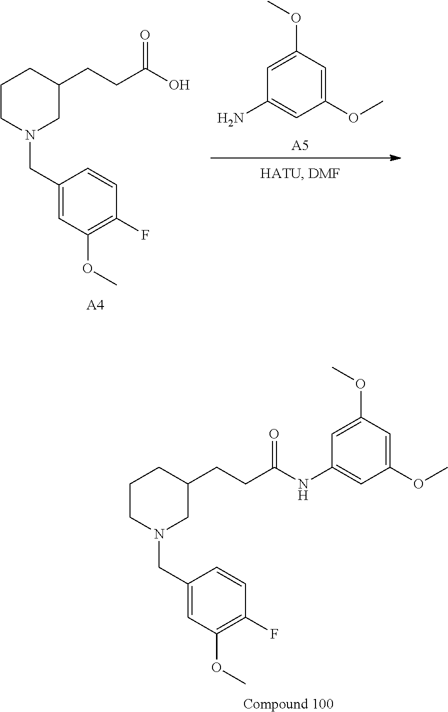

A solution of A4 (1 g, 3.4 mmol, 1 eq), HATU (2.6 g, 6.8 mmol, 2 eq) and DIEA (1.3 g, 10 mmol, 3 eq) was stirred at 25° C. for 30 min, followed by addition of A5 (623 mg, 4.1 mmol, 1.2 eq). The reaction was stirred at 25° C. for 2 hrs, at which point LCMS analysis showed the reaction was complete. The mixture was diluted with water (100 mL) and extracted with ethyl acetate (100 mL*3). The combined organic phase was washed with brine (50 mL*3), dried with anhydrous Na₂SO₄, filtered and concentrated under vacuum. The residue was purified by prep-HPLC (TFA) and the pH was adjusted to 9 with saturated NaHCO₃ (5 mL), followed by extraction with ethyl acetate (50 mL*3) brine (50 mL*1), drying with anhydrous Na₂SO₄, filtration, and concentration under vacuum. Purification by HPLC afforded Compound 100 (250 mg, 580 umol, 17% yield) as a white solid. ¹H NMR: (CDCl₃ 400 MHz) δ 7.09 (dd, J=8.4, 1.2 Hz, 1H) 6.98 (dd, J=11.2, 8.2 Hz, 1H) 6.81-6.86 (m, 1H) 6.79 (d, J=2.2 Hz, 2H) 6.24 (t, J=2.0 Hz, 1H) 3.85 (s, 3H) 3.75 (s, 6H) 3.43-3.51 (m, 2H) 2.78-2.94 (m, 2H) 2.27-2.41 (m, 2H) 1.92-2.04 (m, 1H) 1.80-1.90 (m, 1H) 1.66-1.75 (m, 2H) 1.48-1.65 (m, 4H) 0.86-1.04 (m, 1H). LCMS (ESI+): m/z 431.2 (M+1)⁺, RT: 2.645 min.

### Example 2. Synthesis of N-ethyl-N-((1-(3-methoxyphenethyl)piperidin-3-yl)methyl)-1H-indole-2-carboxamide (Compound 101)

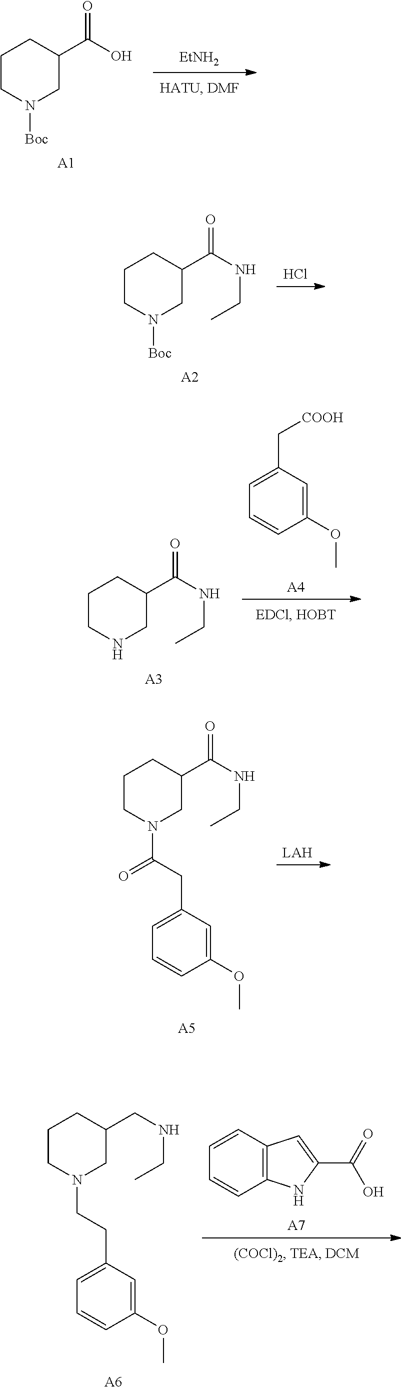

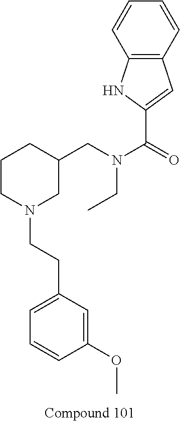

### Step 1: Synthesis of A2

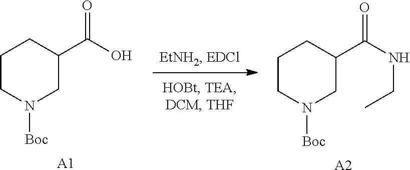

To a solution of ethanamine;hydrochloride (853 mg, 10.5 mmol) in 3:1 of DCM (15 mL):THF (5 mL) was added A1 (2.00 g, 8.72 mmol), TEA (6.18 g, 61.0 mmol), EDCI (3.34 g, 17.4 mmol) and HOBt (2.36 g, 17.4 mmol) at 20° C. The reaction solution was stirred at 20° C. for 12 hrs, after which TLC (Petroleum ether: Ethyl acetate=0:1, R_{f}=0.4) showed that the starting material was consumed. The reaction mixture was poured into water (200 mL) and extracted with DCM/MeOH (v/v=95/5, 70 mL*3). The organic layers were combined and concentrated in vacuo to give a residue. The crude product was purified by column chromatography on silica gel (Petroleum ether: Ethyl acetate=10:1 to 2:1) to give A2 (2.10 g, yield: 93.95%) as a red oil. The product was used directly to the next step. ¹H NMR: (MeOD 400 MHz) δ: ppm 4.07-3.97 (2H, m), 3.21-3.16 (2H, m), 2.80 (2H, m), 2.30-2.24 (1H, m), 1.91-1.88 (1H, m), 1.73-1.64 (2H, m), 1.46 (9H, s), 1.11 (3H, t, J=7.6 Hz).

### Step 2: Synthesis of A3

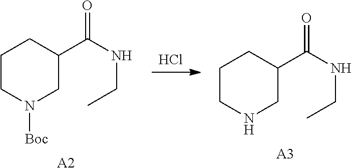

A mixture of A2 (2.10 g, 8.19 mmol) in HCl/EtOAc (50 mL) was stirred at 20° C. for 12 hrs. LCMS showed that the desired MS was detected. The mixture was evaporated under reduced pressure to give crude product A3 (1.50 g, yield: 95.05%, HCl) as a red oil. ¹H NMR: (MeOD 400 MHz) δ: ppm 3.28-3.19 (5H, m), 3.07 (1H, m), 2.76-2.73 (1H, m), 1.99-1.92 (2H, m), 1.82-1.74 (2H, m), 1.12 (3H, t, J=7.6 Hz).

### Step 3: Synthesis of A5

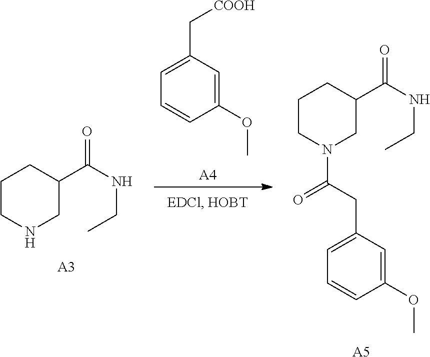

To a solution of A3 (1.40 g, 7.27 mmol, HCl) in 3:1 DCM (15 mL):THF (5 mL) was added 2-(3-methoxyphenyl)acetic acid (1.09 g, 6.54 mmol), EDCI (2.79 g, 14.5 mmol), HOBt (1.96 g, 14.5 mmol) and TEA (5.15 g, 50.9 mmol) at 20° C. The reaction solution was stirred at 20° C. for 12 hrs, until LCMS showed that the desired MS was detected. The reaction was poured into water (150 mL) and extracted with DCM (50 mL*2), and the organic layers were collected and concentrated in vacuo to give a residue, which was purified by HPLC to give A5 (1.60 g, yield: 72.31%) as a colorless solid. ¹H NMR: (CDCl₃ 400 MHz) δ: ppm 7.25 (2H, m), 6.85 (3H, m), 6.26 (1H, br. s), 4.88 (1H, br. s), 4.56 (1H, d, J=11.47 Hz), 3.94 (1H, m), 3.80 (6H, m), 3.55 (2H, m), 3.36 (1H, m), 3.20 (3H, m), 2.57 (1H, m), 2.30 (1H, m), 2.10 (1H, m), 1.72 (2H, m), 1.47 (1H, m), 1.33 (1H, m), 1.11 (3H, m).

### Step 4: Synthesis of A6

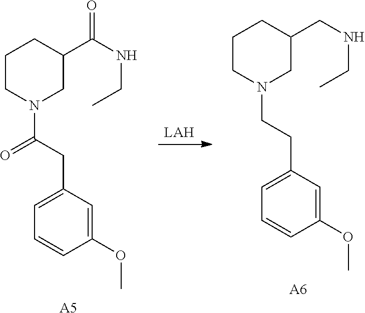 [SMILES: CCNC(=O)C1CCCN(C(=O)Cc2cccc(OC)c2)C1 ; "LAH" ; CCNCC1CCCN(CCc2cccc(OC)c2)C1 ; "A6" ; "A5"]

To a solution of A5 (500 mg, 1.64 mmol) in THF (30 mL) was added LAH (622 mg, 16.4 mmol) at 20° C. The reaction solution was stirred at 75° C. for 12 hrs, until LCMS showed that the desired MS was detected. The reaction was cooled to 0° C. and excess hydride was quenched by drop-wise addition of H₂O (0.622 mL) followed by 15% aq. NaOH (0.622 mL) and then water (1.99 mL). After vigorous stirring for 1 hr at 20° C., the mixture was filtered and the white precipitate was washed with THF (50 mL). The combined organic layers were evaporated under reduced pressure to give crude product, which was purified by prep-HPLC (TFA) to give A6 (600 mg, yield: 72.52%, 2TFA) as a colorless solid. ¹H NMR: (MeOD 400 MHz) δ: ppm 7.26 (1H, t, J=7.94 Hz), 6.84 (3H, m), 3.72 (5H, m), 3.35 (2H, d, J=8.82 Hz), 3.00 (8H, m), 2.34 (1H, br. s), 2.05 (2H, m), 1.87 (1H, m), 1.33 (4H, m).

### Step 5: Synthesis of N-ethyl-N-((1-(3-methoxyphenethyl)piperidin-3-yl)methyl)-1H-indole-2-carboxamide (Compound 101)

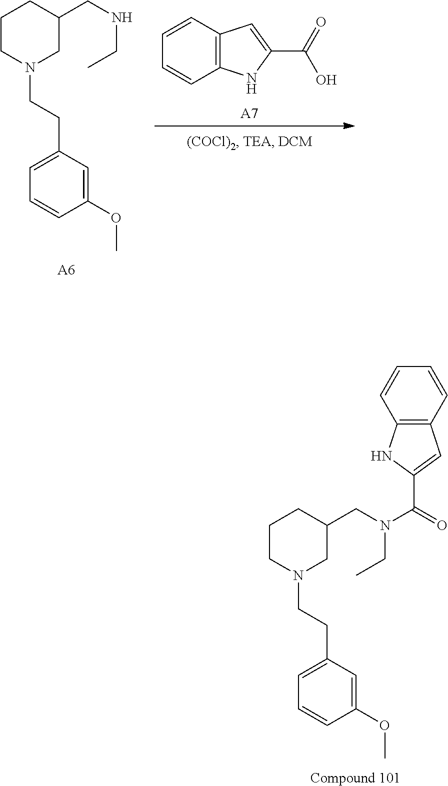

To a solution of A7 (351 mg, 2.18 mmol) and DMF (797 μg, 10.9 umol) in DCM (10 mL) was added drop-wise (COCl)₂ (277 mg, 2.18 mmol) at 0° C. The reaction solution was stirred at 20° C. for 1 hr, after which the solvent was removed. The residue was dissolved in THF (10 mL) and added to a solution of A6 (301 mg, 1.09 mmol) and TEA (221 mg, 2.18 mmol) in THF (10 mL). The mixture was stirred at 20 for 10 hrs until LCMS showed that the desired MS was detected. The mixture was extracted with water (50 mL) and EtOAc (50 mL*2), the organic layers were combined, dried with anhydrous Na₂SO₄ and concentrated in vacuo to give the residue that was purified by prep-HPLC (TFA) to give Compound 101 (50.0 mg, yield: 10.85%) as an off-white solid. 1H NMR: (MeOD 400 MHz) δ: ppm 7.62 (1H, d, J=7.94 Hz), 7.43 (1H, d, J=7.94 Hz), 7.21 (1H, t, J=7.72 Hz), 7.15 (1H, t, J=7.72 Hz), 7.06 (1H, t, J=7.28 Hz), 6.84 (1H, s), 6.72 (3H, m), 3.74 (6H, s), 3.52 (2H, m), 2.94 (2H, br. s), 2.77 (2H, br. s), 2.61 (2H, br. s), 2.14 (2H, br. s), 1.92 (1H, d, J=8.38 Hz), 1.77 (2H, br. s), 1.63 (1H, br. s), 1.30 (2H, m), 1.08 (1H, br. s), 0.88 (1H, br. s). LCMS (ESI+): m/z 420.2 (M+H)⁺.

### Example 3. Synthesis of N-(1-(benzo[d]thiazol-2-yl)piperidin-3-yl)-2-(2-methoxyphenoxy)acetamide (Compound 102)

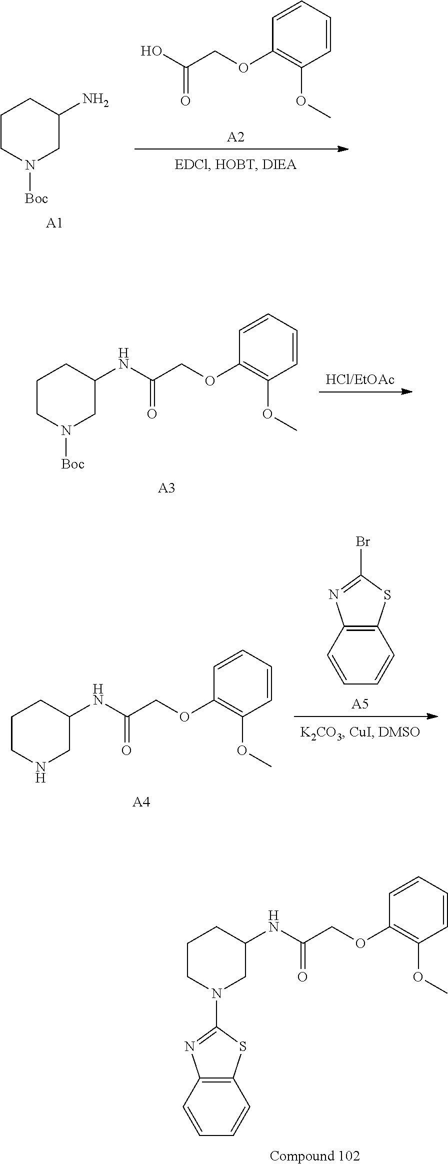

### Step 1: Synthesis of A3

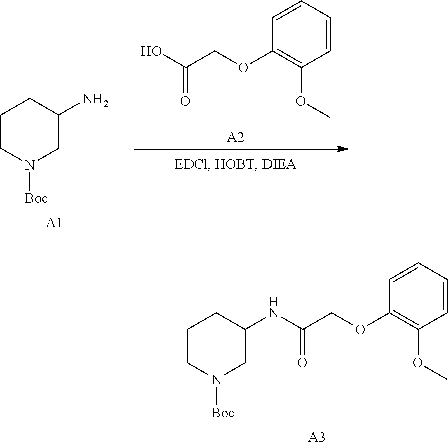

To a mixture of A2 (501 mg, 2.75 mmol), HOBT (507 mg, 3.75 mmol) and EDCI (719 mg, 3.75 mmol) in DMF (5.00 mL) were added DIEA (1.29 g, 10.0 mmol) and A1 (500 mg, 2.50 mmol) at 20° C. The mixture was stirred at 20° C. for 12 h until LCMS showed that the reaction was completed. The mixture was dissolved in EtOAc (30 mL) and washed with water (30 mL*2) and brine (20 mL*2). The organic phase was dried over Na₂SO₄, filtered and the filtrate was concentrated under vacuum to provide a residue, which was purified by silica gel chromatography (petroleum ether/ethyl acetate=30/1 to 1/1) to give A3 (800 mg, yield: 88%) as yellow oil. ¹H NMR: (CDCl₃ 400 MHz) δ: 7.08-7.16 (m, 1H), 7.00-7.06 (m, 1H), 6.91-6.95 (m, 3H), 5.30-5.32 (m, 1H), 4.52-4.55 (m, 2H), 3.97-4.06 (m, 1H), 3.88-3.91 (m, 3H), 3.66-3.76 (m, 1H), 3.46-3.55 (m, 1H), 3.22-3.33 (m, 1H), 1.86-1.95 (m, 1H), 1.56 (d, J=7.0 Hz, 1H), 1.43 (s, 9H), 1.24-1.29 (m, 1H), 1.18-1.23 (m, 1H).

### Step 2: Synthesis of A4

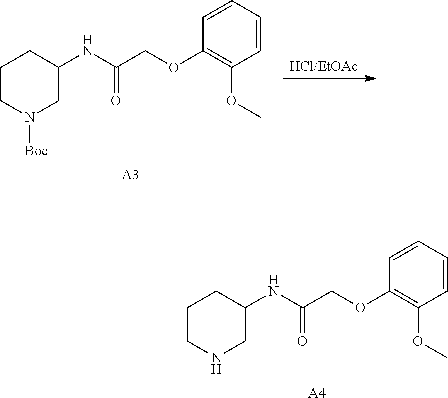 [SMILES: COc1ccccc1OCC(=O)NC1CCCN(C(=O)OC(C)(C)C)C1 ; "HCl/EtOAc" ; "A3" ; COc1ccccc1OCC(=O)NC1CCCNC1 ; "A4"]

A3 (750 mg, 2.06 mmol) was added to HCl/EtOAc (100 mL) at 20° C., and the mixture was stirred at 20° C. for 3 h until LCMS showed that the reaction was complete. The mixture was concentrated in vacuum to afford A4 (500 mg, crude) as a white solid, which was directly in the next step. ¹H NMR: (CDCl₃ 400 MHz) δ: 9.19-9.30 (m, 1H), 9.00-9.11 (m, 1H), 8.21-8.29 (m, 1H), 6.90-7.03 (m, 3H), 6.87 (d, J=7.6 Hz, 1H), 4.48 (s, 2H), 3.98-4.09 (m, 1H), 3.78 (s, 3H), 3.33-3.40 (m, 2H), 3.09-3.21 (m, 2H), 2.72-2.85 (m, 2H), 1.77-1.89 (m, 2H), 1.63-1.74 (m, 1H), 1.45-1.58 (m, 1H).

### Step 3: Synthesis of N-(1-(benzo[d]thiazol-2-yl)piperidin-3-yl)-2-(2-methoxyphenoxy)acetamide (Compound 102)

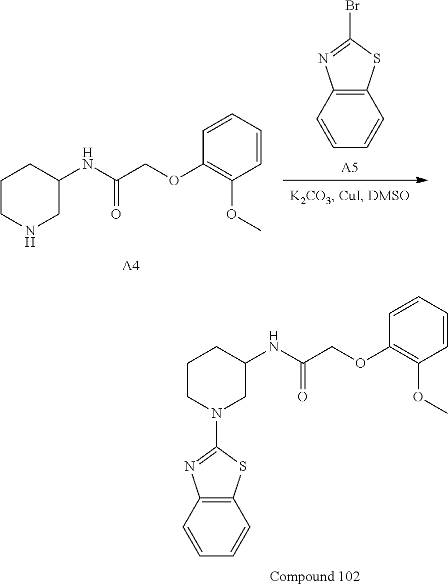

To a mixture of A4 (50.0 mg, 189 umol), K₂CO₃ (131 mg, 946 umol) and CuI (10.8 mg, 56.8 umol) in DMSO (3.00 mL) was added A5 (38.5 mg, 227 umol) at 20° C. The mixture was stirred at 120° C. for 3 h under microwave, until LCMS showed the reaction was complete. Water (30 mL) and EtOAc (40 mL) were added to the mixture. The organic phase was washed with water (20 mL*2) and brine (20 mL*2). The organic layer was dried over Na₂SO₄, filtered and the filtrate was concentrated under vacuum to provide a residue, which was purified by prep-TLC (petroleum ether/ethyl acetate=1/1, R_{f}=0.5) to give Compound 102 (57.0 mg, yield: 74%) as a white solid. ¹H NMR: (CDCl₃ 400 MHz) δ: 7.56-7.59 (m, 1H), 7.51-7.55 (m, 1H), 7.28-7.32 (m, 1H), 7.05-7.10 (m, 1H), 6.93-6.99 (m, 1H), 6.87 (s, 2H), 6.80-6.84 (m, 1H), 4.56 (s, 2H), 4.18-4.27 (m, 1H), 3.84 (br. s., 1H), 3.75 (s, 3H), 3.66 (d, J=4.2 Hz, 2H), 3.43-3.50 (m, 1H), 1.95-2.04 (m, 1H), 1.76 (d, J=7.4 Hz, 3H). LCMS: MS Calcd.: 397.1; MS Found: 398.1 ([M+1]+).

### Example 4: General Protocol A for Synthesis of Exemplary Compounds

General Protocol A to synthesize exemplary compounds of Formula (I) is described in Scheme 1 and the procedures set forth below.

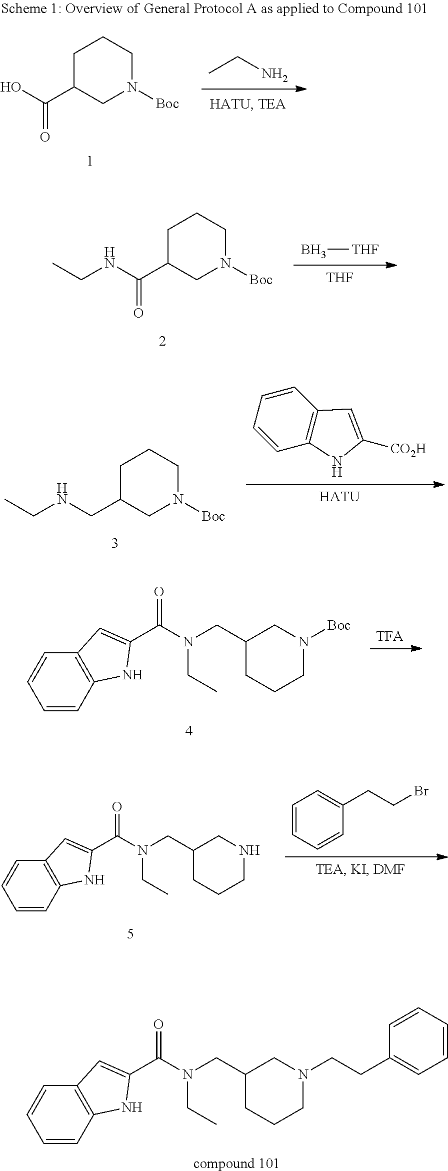

### Synthesis of Exemplary Compounds and Intermediates:

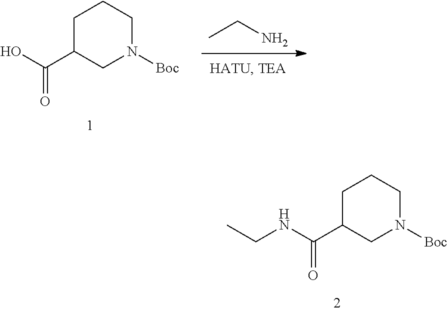

Procedure for the preparation of compound 2: A mixture of acid 1 (5.0 g, 22 mmol, 1.0 eq) and HATU (12.4 g, 32.7 mmol, 1.5 eq) and TEA (3.3 g, 33 mmol, 4.5 mL, 1.5 eq) in DMF (50 mL) was stirred at 25° C. for 0.5 hour, then ethanamine (1.2 g, 26 mmol, 1.2 eq) was added at 25° C., and then the mixture was stirred at 25° C. for 11.5 hours. The reaction was monitored by LCMS and allowed to run until completion. The reaction mixture was diluted with 80 mL of ethyl acetate and washed twice with 80 mL of water. The combined organic layers were washed five times with 100 mL of brine, dried over Na₂SO₄, filtered and concentrated under reduced pressure to give amide 2 (16.0 g, crude) as a brown oil.

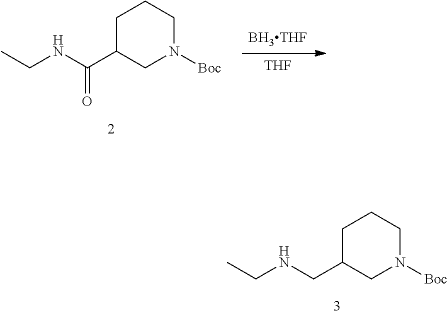 [SMILES: CCNC(=O)C1CCCN(C(=O)OC(C)(C)C)C1 ; "BH3·THF" ; CCNCC1CCCN(C(=O)OC(C)(C)C)C1 ; "THF"]

Procedure for the preparation of compound 3: A mixture of amide 2 (8.0 g, 31.2 mmol, 1.0 eq) in THF (100 mL) was added BH₃.THF (1 M, 93.6 mL, 3.0 eq), and then the mixture was stirred at 60° C. for 4 hours under N₂ atmosphere. The reaction was monitored by LCMS and allowed to run until completion. It was quenched by adding 50 mL of MeOH, concentrated under reduced pressure to give amine 3 (9.0 g, crude) as a white gum and to be used into the next step without further purification.

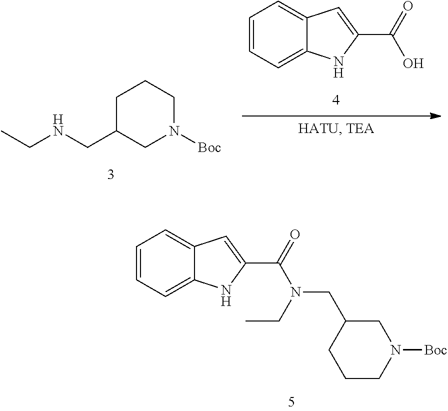 [SMILES: O=C(O)c1cc2ccccc2[nH]1 ; CCNCC1CCCN(C(=O)OC(C)(C)C)C1 ; "HATU, TEA" ; CCN(CC1CCCN(C(=O)OC(C)(C)C)C1)C(=O)c1cc2ccccc2[nH]1]

Procedure for the preparation of compound 5: A mixture of 1H-indole-2-carboxylic acid (3.0 g, 18.6 mmol, 1.0 eq), HATU (8.5 g, 22.3 mmol, 1.2 eq), TEA (5.2 mL, 37.2 mmol, 2.0 eq) in DMF (60 mL) was stirred at 15° C. for 10 mins, then amine 3 (5.0 g, 20.7 mmol, 1.1 eq) was added, and then the mixture was stirred at 15° C. for 12 hrs. The reaction was monitored by LCMS and allowed to run until completion. The reaction mixture was poured into 100 mL of water, stirred for 0.5 hr and filtered to give the filter cake. The residue was washed by petroleum ether (50 mL), and filtered to give 4.5 g of the product amide 5 (11.7 mmol, 62.7% yield) as a white solid.

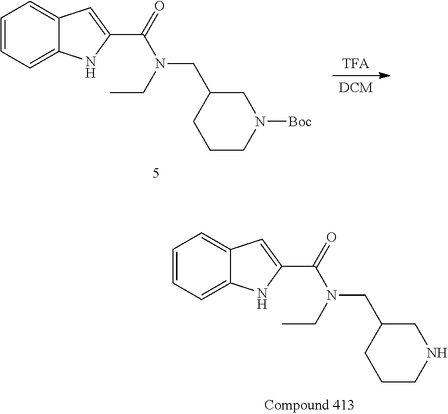

Procedure for the preparation of compound 413: A mixture of amide 5 (1.0 g, 2.6 mmol, 1.0 eq), HCl/MeOH (4 M, 20.0 mL) in DCM (10 mL) was stirred at 20° C. for 4 hours. The reaction was monitored by LCMS and allowed to run until completion. The mixture was evaporated under reduced pressure to give the crude product piperidine 6 (800 mg, crude, HCl salt) as a light brown solid and to be used into the next step without further purification.

¹H NMR (400 MHz, METHANOL-d₄) δ 7.61 (d, J=7.9 Hz, 1H), 7.42 (d, J=8.4 Hz, 1H), 7.20 (t, J=7.7 Hz, 1H), 7.02-7.08 (m, 1H), 6.86 (s, 1H), 3.71 (dd, J=13.7, 8.8 Hz, 2H), 3.45 (d, J=11.9 Hz, 1H), 3.32-3.38 (m, 1H), 3.02-3.09 (m, 1H), 2.89-2.99 (m, 2H), 2.77 (t, J=11.9 Hz, 1H), 2.18-2.33 (m, 1H), 1.88-2.00 (m, 2H), 1.69-1.78 (m, 1H), 1.31 ppm (q, J=7.1 Hz, 4H)

LCMS (ESI+): m/z 286.1 (M+H)

The following compounds were prepared by an analogous method:

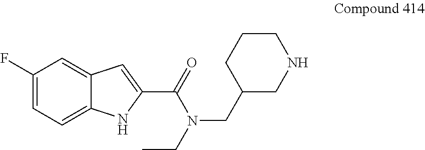 [SMILES: CCN(CC1CCCNC1)C(=O)c1cc2cc(F)ccc2[nH]1]

¹H NMR (400 MHz, METHANOL-d₄) δ ppm 7.39 (dd, J=8.93, 4.52 Hz, 1H) 7.28 (dd, J=9.48, 2.43 Hz, 1H) 6.99 (td, J=9.15, 2.43 Hz, 1H) 6.84 (s, 1H) 3.66-3.84 (m, 3H) 3.29-3.45 (m, 3H) 2.76-3.00 (m, 2H) 2.27 (br s, 1H) 1.88-2.01 (m, 2H) 1.66-1.78 (m, 1H) 1.28-1.43 (m, 4H)

LCMS (ESI+): m/z 304.1 (M+H)

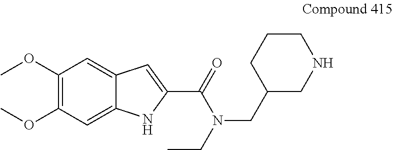 [SMILES: CCN(CC1CCCNC1)C(=O)c1cc2cc(OC)c(OC)cc2[nH]1]

¹H NMR (400 MHz, METHANOL-d₄) δ ppm 7.10 (s, 1H) 6.96 (s, 1H) 6.81 (s, 1H) 3.64-3.94 (m, 8H) 3.28-3.46 (m, 4H) 2.95 (t, J=11.25 Hz, 1H) 2.80 (t, J=11.47 Hz, 1H) 2.28 (d, J=8.38 Hz, 1H) 1.87-2.02 (m, 2H) 1.65-1.78 (m, 1H) 1.26-1.46 (m, 4H)

LCMS (ESI+): m/z 346.1 (M+H)

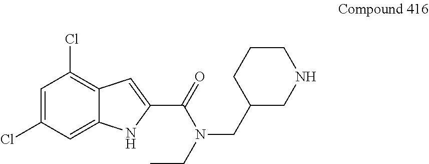 [SMILES: CCN(CC1CCCNC1)C(=O)c1cc2c(Cl)cc(Cl)cc2[nH]1]

¹H NMR (400 MHz, DMSO-d₆) δ ppm 7.41 (s, 1H) 7.21 (d, 1H) 6.77 (s, 1H) 3.45-3.69 (m, 4H) 3.07 (br. s., 2H) 2.61-2.70 (m, 2H) 2.05 (br. s., 1H) 1.70 (br. s., 2H) 1.51 (br. s., 1H) 1.09-1.25 (m, 4H)

LCMS (ESI+): m/z 354.1 (M+H)

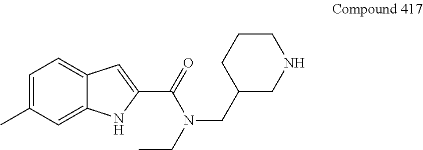 [SMILES: CCN(CC1CCCNC1)C(=O)c1cc2ccc(C)cc2[nH]1]

¹H NMR (400 MHz, METHANOL-d₄) δ ppm 7.50 (br d, J=8.2 Hz, 1H), 7.23 (br s, 1H), 6.92 (br d, J=8.4 Hz, 1H), 6.89-6.95 (m, 1H), 6.84 (br s, 1H), 3.83 (br s, 1H), 3.67-3.79 (m, 2H), 3.43 (br s, 2H), 2.70-3.03 (m, 3H), 2.43 (s, 3H), 2.28 (br s, 1H), 1.95 (br t, J=16.3 Hz, 2H), 1.73 (br d, J=11.5 Hz, 1H), 1.41 (br s, 1H), 1.34 (br t, J=5.7 Hz, 3H)

LCMS (ESI+): m/z 300.1 (M+H)

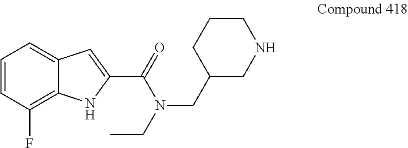 [SMILES: CCN(CC1CCCNC1)C(=O)c1cc2cccc(F)c2[nH]1]

¹H NMR (400 MHz, METHANOL-d₄) δ ppm 7.43 (br d, J=7.7 Hz, 1H), 6.88-7.07 (m, 3H), 3.67-3.87 (m, 3H), 3.41 (br d, J=17.6 Hz, 2H), 2.72-3.07 (m, 3H), 2.28 (br s, 1H), 1.90-2.05 (m, 2H), 1.74 (br d, J=11.0 Hz, 1H), 1.42 (br s, 1H), 1.32 (br t, J=6.6 Hz, 3H)

LCMS (ESI+): m/z 304.1 (M+H)

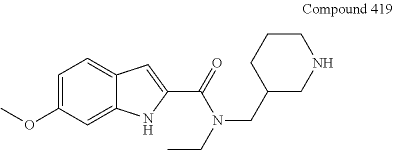 [SMILES: CCN(CC1CCCNC1)C(=O)c1cc2ccc(OC)cc2[nH]1]

¹H NMR (400 MHz, METHANOL-d₄) δ ppm 7.49 (d, J=8.38 Hz, 1H) 6.93 (d, J=1.76 Hz, 1H) 6.85 (s, 1H) 6.74 (dd, J=8.82, 2.21 Hz, 1H) 3.69-3.90 (m, 6H) 3.32-3.47 (m, 2H) 2.75-3.05 (m, 3H) 2.29 (br. s., 1H) 1.87-2.07 (m, 2H) 1.68-1.82 (m, 1H) 1.31-1.46 (m, 4H)

LCMS (ESI+): m/z 316.1 (M+H)

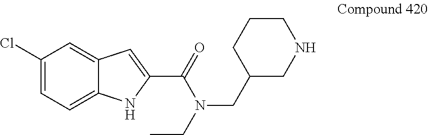 [SMILES: CCN(CC1CCCNC1)C(=O)c1cc2cc(Cl)ccc2[nH]1]

¹H NMR (400 MHz, METHANOL-d₄) δ ppm 7.63 (br. s., 1H) 7.42 (d, J=8.38 Hz, 1H) 7.19 (d, J=8.38 Hz, 1H) 6.86 (br. s., 1H) 4.53 (d, J=6.17 Hz, 1H) 3.62-3.91 (m, 3H) 3.41 (br. s., 2H) 2.91-3.06 (m, 1H) 2.82 (br. s., 1H) 2.19-2.38 (m, 1H) 1.96 (t, J=15.22 Hz, 2H) 1.74 (d, J=11.91 Hz, 1H) 1.42 (br. s., 1H) 1.34 (br. s., 3H)

LCMS (ESI+): m/z 320.1 (M+H)

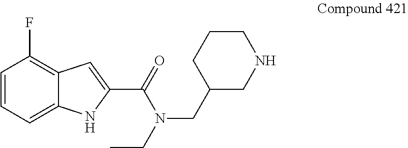 [SMILES: CCN(CC1CCCNC1)C(=O)c1cc2c(F)cccc2[nH]1]

¹H NMR (400 MHz, METHANOL-d₄) δ ppm 7.21-7.27 (m, 1H) 7.16 (td, J=7.99, 5.18 Hz, 1H) 6.89 (s, 1H) 6.74 (ddd, J=10.58, 7.72, 0.66 Hz, 1H) 3.71 (br dd, J=14.00, 8.49 Hz, 3H) 3.30-3.49 (m, 3H) 2.76-3.01 (m, 2H) 2.28 (br s, 1H) 1.88-2.02 (m, 2H) 1.72 (br d, J=10.80 Hz, 1H) 1.29-1.46 (m, 4H)

LCMS (ESI+): m/z 304.1 (M+H)

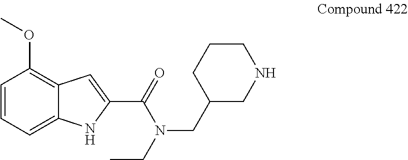 [SMILES: CCN(CC1CCCNC1)C(=O)c1cc2c(OC)cccc2[nH]1]

¹H NMR (400 MHz, METHANOL-d₄) δ ppm 7.12-7.19 (m, 1H) 7.03 (br d, J=9.04 Hz, 1H) 6.94 (s, 1H) 6.53 (br d, J=6.84 Hz, 1H) 3.93 (s, 3H) 3.84 (br s, 1H) 3.69-3.78 (m, 2H) 3.43 (br s, 1H) 3.31-3.35 (m, 2H) 2.96 (brt, J=11.36 Hz, 1H) 2.81 (br s, 1H) 2.29 (br s, 1H) 1.89-2.02 (m, 2H) 1.73 (br d, J=11.25 Hz, 1H) 1.36 (br t, J=6.17 Hz, 3H)

LCMS (ESI+): m/z 316.2 (M+H)

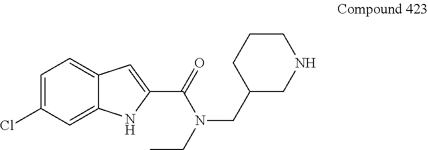 [SMILES: CCN(CC1CCCNC1)C(=O)c1cc2ccc(Cl)cc2[nH]1]

¹H NMR (400 MHz, METHANOL-d₄) δ ppm 7.60 (br d, J=8.60 Hz, 1H) 7.45 (s, 1H) 7.06 (br d, J=8.16 Hz, 1H) 6.90 (br s, 1H) 3.79-3.89 (m, 1H) 3.69-3.77 (m, 2H) 3.41 (br s, 1H) 3.33 (br s, 2H) 2.91-3.04 (m, 1H) 2.82 (br s, 1H) 2.28 (br s, 1H) 1.89-2.02 (m, 2H) 1.73 (br d, J=11.25 Hz, 1H) 1.38-1.48 (m, 1H) 1.31-1.37 (m, 3H)

LCMS (ESI+): m/z 320.1 (M+H)

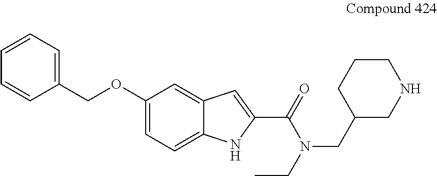 [SMILES: CCN(CC1CCCNC1)C(=O)c1cc2cc(OCc3ccccc3)ccc2[nH]1]

¹H NMR (400 MHz, METHANOL-d₄) δ ppm 7.46 (br d, J=7.50 Hz, 2H) 7.33-7.40 (m, 3H) 7.27-7.32 (m, 1H) 7.17 (s, 1H) 6.96-7.01 (m, 1H) 6.81 (s, 1H) 5.09 (s, 2H) 3.82 (br s, 1H) 3.73 (br dd, J=13.89, 9.04 Hz, 2H) 3.42 (br s, 1H) 3.32 (br s, 2H) 2.91-3.02 (m, 1H) 2.81 (br s, 1H) 2.28 (br s, 1H) 1.88-2.05 (m, 2H) 1.73 (br d, J=12.79 Hz, 1H) 1.38-1.48 (m, 1H) 1.34 (br t, J=6.50 Hz, 3H)

LCMS (ESI+): m/z 392.2 (M+H)

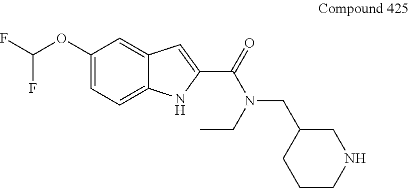 [SMILES: CCN(CC1CCCNC1)C(=O)c1cc2cc(OC(F)F)ccc2[nH]1]

¹H NMR (400 MHz, METHANOL-d₄) δ ppm 7.38-7.46 (m, 2H) 7.05 (dd, J=8.77, 1.75 Hz, 1H) 6.86-6.92 (m, 1H) 6.50-6.74 (m, 1H) 3.72 (br dd, J=13.59, 8.33 Hz, 3H) 3.33-3.40 (m, 2H) 2.72-3.04 (m, 2H) 2.20-2.37 (m, 1H) 1.89-2.04 (m, 2H) 1.73 (br d, J=12.72 Hz, 1H) 1.27-1.48 (m, 5H)

LCMS (ESI+): m/z 352.1 (M+H)

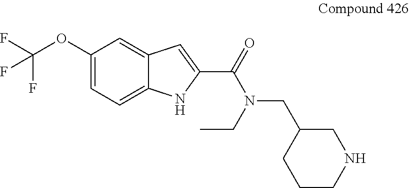 [SMILES: CCN(CC1CCCNC1)C(=O)c1cc2cc(OC(F)(F)F)ccc2[nH]1]

¹H NMR (400 MHz, METHANOL-d₄) δ ppm 7.50 (s, 1H) 7.44 (d, J=8.8 Hz, 1H) 7.09 (br d, J=8.8 Hz, 1H) 6.88 (s, 1H) 3.67 (br dd, J=8.6, 13.9 Hz, 3H) 3.46 (br s, 1H) 3.29-3.23 (m, 2H) 2.96-2.69 (m, 2H) 2.24 (br s, 1H) 1.97-1.84 (m, 2H) 1.75-1.62 (m, 1H) 1.41-1.24 (m, 4H)

LCMS (ESI+): m/z 370.1 (M+H)

### Synthesis of Compound 101:

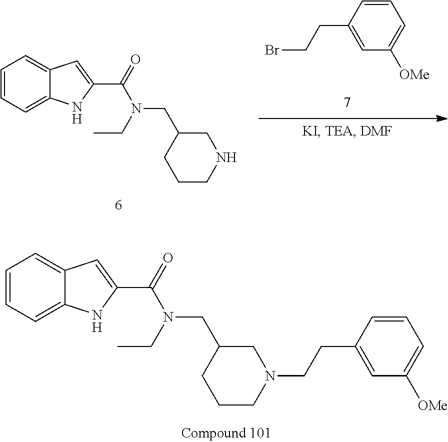 [SMILES: CCN(CC1CCCNC1)C(=O)c1cc2ccccc2[nH]1 ; COc1cccc(CCBr)c1 ; "KI, TEA, DMF" ; CCN(CC1CCCN(CCc2cccc(OC)c2)C1)C(=O)c1cc2ccccc2[nH]1]

Alternate procedure “A” for preparation of compound 101: A mixture of amine 6 (600 mg, 1.9 mmol, 1.0 eq, HCl), alkyl halide 7 (440 mg, 2.1 mmol, 1.1 eq), TEA (376 mg, 3.7 mmol, 2.0 eq), KI (31 mg, 186 μmol, 0.1 eq) in DMF (8 mL) was degassed and purged with N₂ for 3 times, and then the mixture was stirred at 30° C. for 24 hours under N₂ atmosphere. The reaction was monitored by LCMS and TLC and allowed to run until completion. The reaction mixture was partitioned between 30 mL of water and 30 mL of ethyl acetate. The organic phase was separated, washed twice with 30 mL of water, dried over Na₂SO₄, filtered and concentrated under reduced pressure to give a residue. The residue was purified by flash silica gel chromatography (ISCO®; 20 g SepaFlash® Silica Flash Column, eluting with ethyl acetate @ 80 mL/min) to give 297 mg compound 101 (38% yield) as a white solid.

¹H NMR (400 MHz, METHANOL-d₄) δ ppm 7.60 (d, J=8.38 Hz, 1H) 7.41 (d, J=8.38 Hz, 1H) 7.19 (t, J=7.28 Hz, 1H) 7.13 (t, J=7.94 Hz, 1H) 7.04 (t, J=7.50 Hz, 1H) 6.81 (s, 1H) 6.65-6.76 (m, 3H) 3.37-3.83 (m, 7H) 2.68-3.01 (m, 4H) 2.58 (d, J=7.50 Hz, 2H) 2.11 (br. s., 2H) 1.68-1.94 (m, 3H) 1.60 (br. s., 1H) 1.28 (t, J=6.84 Hz, 3H) 1.03 (d, J=10.58 Hz, 1H)

LCMS (ESI+): m/z 420.2 (M+H)

The following compounds were prepared analogously using General Protocol A. Some analogs were isolated as TFA salts from the chromatographic purification.

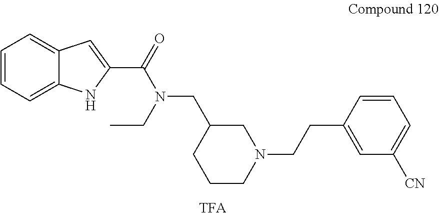 [SMILES: CCN(CC1CCCN(CCc2cccc(C#N)c2)C1)C(=O)c1cc2ccccc2[nH]1]

¹H NMR (400 MHz, METHANOL-d₄) δ ppm 7.67-7.75 (m, 1H) 7.57-7.66 (m, 3H) 7.41-7.54 (m, 2H) 7.18-7.28 (m, 1H) 7.04-7.12 (m, 1H) 6.82-6.94 (m, 1H) 3.60-3.71 (m, 2H) 3.34-3.44 (m, 2H) 3.02-3.21 (m, 2H) 1.68-1.77 (m, 1H) 1.66 (d, J=7.2 Hz, 3H) 1.40-1.55 (m, 2H) 1.21-1.38 (m, 6H) 0.94-1.03 (m, 2H)

LCMS (ESI+): m/z 415.1 (M+H)

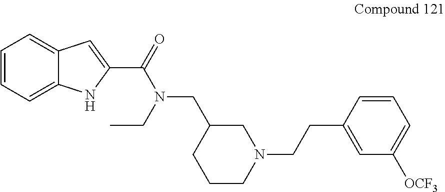 [SMILES: CCN(CC1CCCN(CCc2cccc(OC(F)(F)F)c2)C1)C(=O)c1cc2ccccc2[nH]1]

¹H NMR (400 MHz, METHANOL-d₄) δ ppm 7.62 (d, J=8.4 Hz, 1H) 7.43 (d, J=8.4 Hz, 1H) 7.29-7.38 (m, 1H) 7.16-7.26 (m, 2H) 7.01-7.14 (m, 2H) 6.83 (s, 1H) 3.37-3.85 (m, 4H) 2.83 (m., 4H) 2.60 (d, J=8 Hz, 2H) 2.11 (s., 2H) 1.76 (m., 4H) 1.29 (s., 3H) 0.87-1.16 (m, 2H)

LCMS (ESI+): m/z 474.2 (M+H)

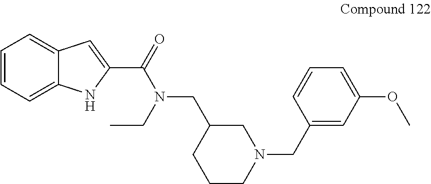 [SMILES: CCN(CC1CCCN(Cc2cccc(OC)c2)C1)C(=O)c1cc2ccccc2[nH]1]

¹H NMR (METHANOL-D₄, 400 MHz) δ ppm 7.61 (d, J=7.9 Hz, 1H), 7.40-7.48 (m, 1H), 7.32 (t, J=7.9 Hz, 1H), 7.22 (t, J=7.3 Hz, 1H), 6.95-7.12 (m, 4H), 6.77 (s, 1H), 4.18-4.36 (m, 3H), 3.73 (s, 3H), 3.35-3.49 (m, 3H), 3.08 (d, J=16.8 Hz, 1H), 2.90 (t, J=12.1 Hz, 1H), 2.71-2.82 (m, 1H), 2.31 (br. s., 1H), 1.87-2.04 (m, 3H), 1.72-1.83 (m, 1H), 1.35 (t, J=7.1 Hz, 1H), 1.27 (t, J=7.1 Hz, 3H)

LCMS (ESI+): m/z 406.2 (M+H)

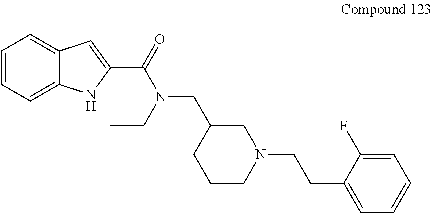 [SMILES: CCN(CC1CCCN(CCc2ccccc2F)C1)C(=O)c1cc2ccccc2[nH]1]

¹H NMR (400 MHz, METHANOL-d₄) δ ppm 7.61 (d, J=7.94 Hz, 1H) 7.42 (d, J=8.38 Hz, 1H) 7.17-7.32 (m, 3H) 7.01-7.14 (m, 3H) 6.87 (br. s., 1H) 3.73 (br. s., 4H) 3.31-3.51 (m, 4H) 3.04 (br. s., 4H) 2.29 (br. s., 1H) 1.68-2.02 (m, 3H) 1.33 (br. s., 4H)

LCMS (ESI+): m/z 408.2 (M+H)

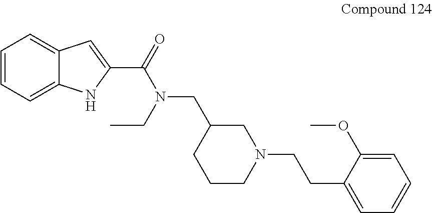 [SMILES: CCN(CC1CCCN(CCc2ccccc2OC)C1)C(=O)c1cc2ccccc2[nH]1]

¹H NMR (400 MHz, METHANOL-d₄) δ ppm 7.62 (d, J=8.38 Hz, 1H) 7.42 (d, J=8.38 Hz, 1H) 7.12-7.25 (m, 3H) 7.00-7.09 (m, 1H) 6.82-6.96 (m, 3H) 3.64-3.93 (m, 7H) 3.35 (br. s., 4H) 2.92-3.20 (m, 4H) 2.33 (br. s., 1H) 1.70-2.03 (m, 3H) 1.33 (t, J=6.39 Hz, 4H)

LCMS (ESI+): m/z 420.2 (M+H)

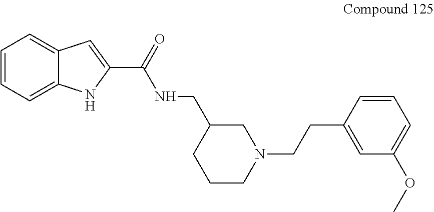 [SMILES: COc1cccc(CCN2CCCC(CNC(=O)c3cc4ccccc4[nH]3)C2)c1]

¹H NMR (400 MHz, METHANOL-d₄) δ ppm 7.59 (d, J=8.38 Hz, 1H) 7.42 (d, J=8.38 Hz, 1H) 7.16-7.24 (m, 2H) 7.01-7.08 (m, 2H) 6.75-6.86 (m, 3H) 3.73 (s, 3H) 3.62 (br. s., 2H) 3.31-3.44 (m, 5H) 2.98-3.06 (m, 2H) 2.86-2.96 (m, 1H) 2.77 (t, J=12.57 Hz, 1H) 2.17 (br. s., 1H) 1.91-2.08 (m, 2H) 1.79 (d, J=11.91 Hz, 1H) 1.25-1.39 (m, 1H)

LCMS (ESI+): m/z 392.2 (M+H)

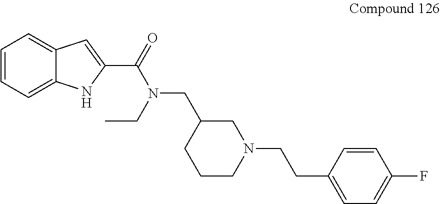 [SMILES: CCN(CC1CCCN(CCc2ccc(F)cc2)C1)C(=O)c1cc2ccccc2[nH]1]

¹H NMR (400 MHz, METHANOL-d₄) δ ppm 7.61 (d, J=7.94 Hz, 1H) 7.42 (d, J=8.38 Hz, 1H) 7.16-7.27 (m, 3H) 6.95-7.08 (m, 3H) 6.86 (br. s., 1H) 3.73 (br. s., 4H) 3.44 (br. s., 4H) 2.91 (br. s., 4H) 2.26 (br. s., 1H) 1.63-1.97 (m, 3H) 1.31 (br. s., 4H)

LCMS (ESI+): m/z 408.2 (M+H)

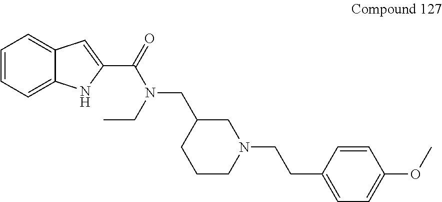 [SMILES: CCN(CC1CCCN(CCc2ccc(OC)cc2)C1)C(=O)c1cc2ccccc2[nH]1]

¹H NMR (400 MHz, METHANOL-d₄) δ ppm 7.59-7.65 (m, 1H) 7.39-7.46 (m, 1H) 7.12-7.26 (m, 3H) 7.06 (t, J=7.28 Hz, 1H) 6.79-6.90 (m, 3H) 3.60-3.85 (m, 7H) 3.31-3.57 (m, 4H) 2.77-3.11 (m, 4H) 2.34 (br. s., 1H) 1.73-2.05 (m, 3H) 1.28-1.42 (m, 4H)

LCMS (ESI+): m/z 420.2 (M+H)

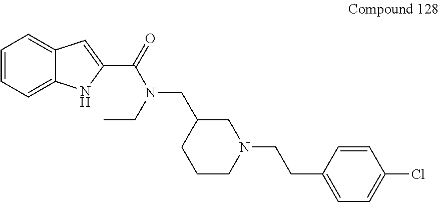 [SMILES: CCN(CC1CCCN(CCc2ccc(Cl)cc2)C1)C(=O)c1cc2ccccc2[nH]1]

¹H NMR (400 MHz, METHANOL-d₄) δ ppm 7.59-7.66 (m, 1H) 7.39-7.46 (m, 1H) 7.19-7.33 (m, 5H) 7.02-7.11 (m, 1H) 6.88 (s, 1H) 3.59-3.83 (m, 4H) 3.32-3.58 (m, 4H) 2.78-3.15 (m, 4H) 2.34 (br. s., 1H) 1.74-2.07 (m, 3H) 1.26-1.43 (m, 4H)

LCMS (ESI+): m/z 424.2 (M+H)

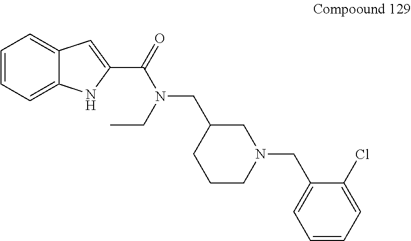 [SMILES: CCN(CC1CCCN(Cc2ccccc2Cl)C1)C(=O)c1cc2ccccc2[nH]1]

¹H NMR (400 MHz, METHANOL-d4) δ ppm 7.58 (d, J=7.94 Hz, 1H) 7.28-7.47 (m, 3H) 7.12-7.23 (m, 3H) 7.00-7.07 (m, 1H) 6.74 (s, 1H) 3.44-3.71 (m, 6H) 2.80 (br. s., 2H) 1.93-2.24 (m, 3H) 1.48-1.80 (m, 3H) 1.23 (br. s., 3H) 1.09 (br. s., 1H)

LCMS (ESI+): m/z 410.2 (M+H)

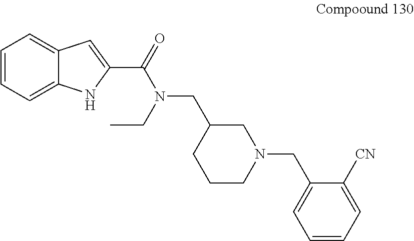 [SMILES: CCN(CC1CCCN(Cc2ccccc2C#N)C1)C(=O)c1cc2ccccc2[nH]1]

¹H NMR (400 MHz, METHANOL-d₄) δ ppm 7.66 (d, J=7.50 Hz, 1H) 7.46-7.61 (m, 3H) 7.34-7.41 (m, 2H) 7.19 (t, J=7.50 Hz, 1H) 7.01-7.07 (m, 1H) 6.74 (s, 1H) 3.44-3.71 (m, 6H) 2.76 (br. s., 2H) 1.93-2.23 (m, 3H) 1.46-1.77 (m, 3H) 1.18-1.28 (m, 3H) 1.09 (br. s., 1H)

LCMS (ESI+): m/z 401.2 (M+H)

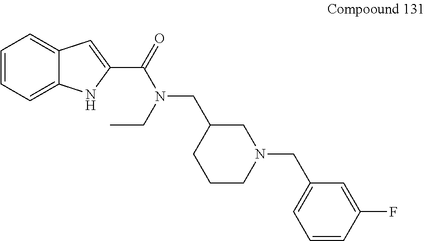 [SMILES: CCN(CC1CCCN(Cc2cccc(F)c2)C1)C(=O)c1cc2ccccc2[nH]1]

¹H NMR (400 MHz, METHANOL-d₄) δ ppm 7.59 (d, J=7.94 Hz, 1H) 7.39 (d, J=8.38 Hz, 1H) 7.15-7.29 (m, 2H) 7.00-7.10 (m, 3H) 6.95 (t, J=8.38 Hz, 1H) 6.73 (s, 1H) 3.45-3.71 (m, 6H) 2.79 (br. s., 2H) 2.06 (br. s., 2H) 1.48-1.94 (m, 4H) 1.23 (t, J=6.84 Hz, 3H) 1.06 (br. s., 1H)

LCMS (ESI+): m/z 394.2 (M+H)

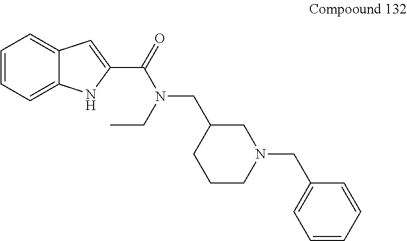 [SMILES: CCN(CC1CCCN(Cc2ccccc2)C1)C(=O)c1cc2ccccc2[nH]1]

¹H NMR (400 MHz, METHANOL-d₄) δ ppm 7.60 (d, J=7.94 Hz, 1H) 7.25-7.45 (m, 6H) 7.21 (t, J=7.72 Hz, 1H) 7.03-7.09 (m, 1H) 6.76 (s, 1H) 3.41-4.06 (m, 7H) 2.97-3.19 (m, 2H) 2.23 (br. s., 2H) 1.59-1.91 (m, 3H) 1.26 (br. s., 4H)

LCMS (ESI+): m/z 376.2 (M+H)

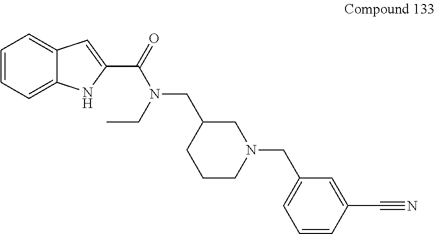 [SMILES: CCN(CC1CCCN(Cc2cccc(C#N)c2)C1)C(=O)c1cc2ccccc2[nH]1]

¹H NMR (400 MHz, METHANOL-d₄) δ ppm 7.68 (br. s., 1H) 7.59 (t, J=7.28 Hz, 3H) 7.40 (d, J=7.94 Hz, 2H) 7.19 (t, J=7.50 Hz, 1H) 7.01-7.08 (m, 1H) 6.74 (s, 1H) 3.36-3.82 (m, 7H) 2.80 (br. s., 2H) 2.14 (br. s., 2H) 1.49-1.82 (m, 3H) 1.24 (t, J=6.39 Hz, 3H) 1.09 (br. s., 1H)

LCMS (ESI+): m/z 401.2 (M+H)

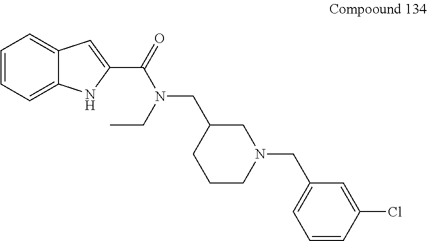 [SMILES: CCN(CC1CCCN(Cc2cccc(Cl)c2)C1)C(=O)c1cc2ccccc2[nH]1]

¹H NMR (400 MHz, METHANOL-d₄) δ ppm 7.59 (d, J=7.94 Hz, 1H) 7.30-7.43 (m, 2H) 7.14-7.26 (m, 4H) 7.04 (t, J=7.50 Hz, 1H) 6.73 (s, 1H) 3.34-3.77 (m, 7H) 2.81 (br. s., 2H) 2.11 (br. s., 2H) 1.50-1.80 (m, 3H) 1.23 (t, J=6.62 Hz, 3H) 1.07 (br. s., 1H)

LCMS (ESI+): m/z 410.2 (M+H)

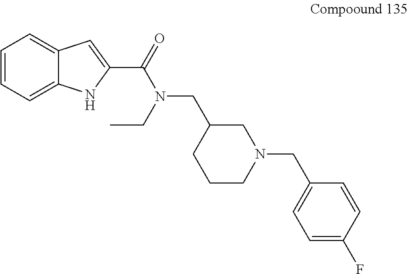 [SMILES: CCN(CC1CCCN(Cc2ccc(F)cc2)C1)C(=O)c1cc2ccccc2[nH]1]

¹H NMR (400 MHz, METHANOL-d₄) δ ppm 7.60 (d, J=7.94 Hz, 1H) 7.41 (d, J=7.94 Hz, 3H) 7.20 (t, J=7.50 Hz, 1H) 6.94-7.09 (m, 3H) 6.75 (s, 1H) 3.33-3.89 (m, 7H) 2.97 (br. s., 2H) 2.17 (br. s., 2H) 1.54-1.85 (m, 3H) 1.25 (br. s., 4H)

LCMS (ESI+): m/z 394.2 (M+H)

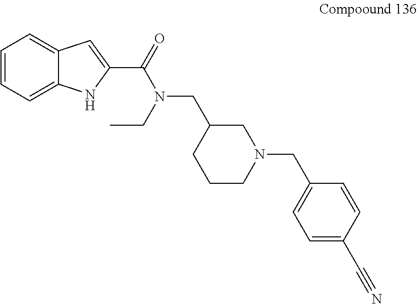 [SMILES: CCN(CC1CCCN(Cc2ccc(C#N)cc2)C1)C(=O)c1cc2ccccc2[nH]1]

¹H NMR (400 MHz, METHANOL-d₄) δ ppm 7.34-7.68 (m, 6H) 7.20 (t, J=7.50 Hz, 1H) 7.06 (t, J=7.50 Hz, 1H) 6.73 (s, 1H) 3.38-3.90 (m, 7H) 2.83 (br. s., 2H) 2.14 (br. s., 2H) 1.49-1.83 (m, 3H) 1.19-1.29 (m, 3H) 1.10 (br. s., 1H)

LCMS (ESI+): m/z 394.2 (M+H)

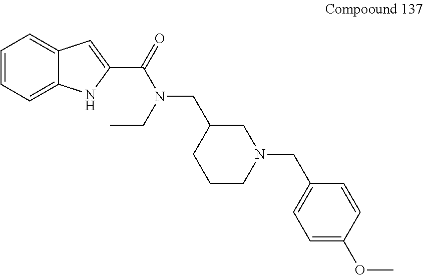 [SMILES: CCN(CC1CCCN(Cc2ccc(OC)cc2)C1)C(=O)c1cc2ccccc2[nH]1]

¹H NMR (400 MHz, METHANOL-d₄) δ ppm 7.60 (d, J=7.94 Hz, 1H) 7.41 (d, J=7.94 Hz, 1H) 7.20 (t, J=7.50 Hz, 3H) 7.05 (t, J=7.50 Hz, 1H) 6.69-6.85 (m, 3H) 3.37-3.84 (m, 10H) 2.97 (br. s., 2H) 2.15 (br. s., 2H) 1.55-1.85 (m, 3H) 1.24 (t, J=6.84 Hz, 4H)

LCMS (ESI+): m/z 406.2 (M+H)

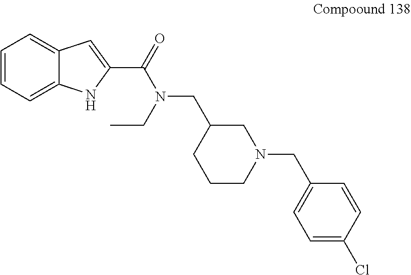 [SMILES: CCN(CC1CCCN(Cc2ccc(Cl)cc2)C1)C(=O)c1cc2ccccc2[nH]1]

¹H NMR (400 MHz, METHANOL-d₄) δ ppm 7.60 (d, J=7.94 Hz, 1H) 7.40 (d, J=7.94 Hz, 1H) 7.16-7.35 (m, 5H) 7.02-7.08 (m, 1H) 6.74 (s, 1H) 3.37-3.86 (m, 7H) 2.69-3.01 (m, 2H) 2.13 (br. s., 2H) 1.52-1.82 (m, 3H) 1.00-1.29 (m, 4H)

LCMS (ESI+): m/z 410.2 (M+H)

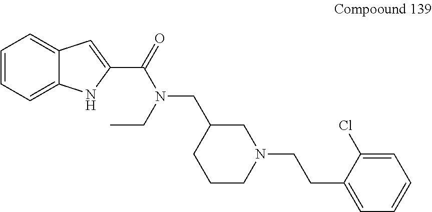 [SMILES: CCN(CC1CCCN(CCc2ccccc2Cl)C1)C(=O)c1cc2ccccc2[nH]1]

¹H NMR (400 MHz, METHANOL-d₄) δ ppm 7.61 (d, J=8.38 Hz, 1H) 7.41 (d, J=8.38 Hz, 1H) 7.25-7.36 (m, 2H) 7.14-7.24 (m, 3H) 7.01-7.08 (m, 1H) 6.85 (br. s., 1H) 3.33-3.94 (m, 7H) 2.76-3.18 (m, 5H) 2.22 (br. s., 1H) 1.60-1.91 (m, 3H) 1.30 (br. s., 4H)

LCMS (ESI+): m/z 424.3 (M+H)

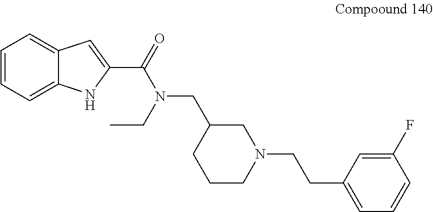 [SMILES: CCN(CC1CCCN(CCc2cccc(F)c2)C1)C(=O)c1cc2ccccc2[nH]1]

¹H NMR (400 MHz, METHANOL-d₄) δ ppm 7.61 (d, J=8.38 Hz, 1H) 7.41 (d, J=7.94 Hz, 1H) 7.16-7.30 (m, 2H) 6.94-7.09 (m, 3H) 6.80-6.93 (m, 2H) 3.34-3.89 (m, 6H) 2.90 (br. s., 6H) 2.24 (br. s., 1H) 1.60-1.92 (m, 3H) 1.31 (br. s., 4H)

LCMS (ESI+): m/z 408.3 (M+H)

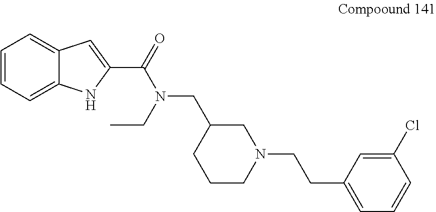 [SMILES: CCN(CC1CCCN(CCc2cccc(Cl)c2)C1)C(=O)c1cc2ccccc2[nH]1]

¹H NMR (400 MHz, METHANOL-d₄) δ ppm 7.61 (d, J=7.94 Hz, 1H) 7.41 (d, J=8.38 Hz, 1H) 7.10-7.28 (m, 5H) 7.01-7.08 (m, 1H) 6.84 (br. s., 1H) 3.34-3.87 (m, 6H) 2.70-3.18 (m, 6H) 2.19 (br. s., 1H) 1.57-1.88 (m, 3H) 1.06-1.36 (m, 4H)

LCMS (ESI+): m/z 424.2 (M+H)

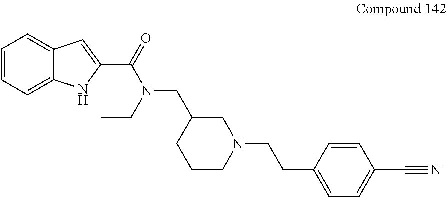 [SMILES: CCN(CC1CCCN(CCc2ccc(C#N)cc2)C1)C(=O)c1cc2ccccc2[nH]1]

¹H NMR (400 MHz, METHANOL-d₄) δ ppm 7.54-7.62 (m, 3H) 7.39 (t, J=7.94 Hz, 3H) 7.19 (t, J=7.72 Hz, 1H) 7.00-7.08 (m, 1H) 6.82 (s, 1H) 3.36-3.85 (m, 5H) 2.87 (br. s., 4H) 2.64 (br. s., 2H) 2.14 (br. s., 2H) 1.53-1.82 (m, 3H) 1.03-1.36 (m, 4H)

LCMS (ESI+): m/z 415.3 (M+H)

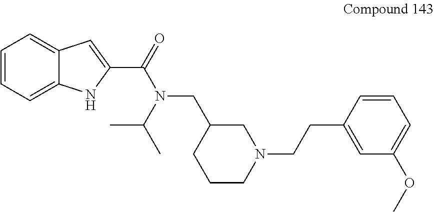 [SMILES: COc1cccc(CCN2CCCC(CN(C(=O)c3cc4ccccc4[nH]3)C(C)C)C2)c1]

¹H NMR (400 MHz, METHANOL-d₄) δ ppm 7.57-7.65 (m, 1H) 7.39-7.46 (m, 1H) 7.14-7.25 (m, 2H) 7.02-7.10 (m, 1H) 6.75-6.85 (m, 4H) 3.67-3.78 (m, 3H) 3.53-3.65 (m, 2H) 3.42-3.51 (m, 1H) 3.30-3.40 (m, 4H) 2.76-3.18 (m, 4H) 2.36 (br. s., 1H) 1.70-2.06 (m, 3H) 1.26-1.36 (m, 7H)

LCMS (ESI+): m/z 434.2 (M+H)

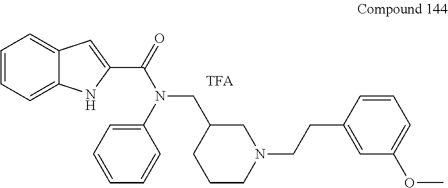 [SMILES: COc1cccc(CCN2CCCC(CN(C(=O)c3cc4ccccc4[nH]3)c3ccccc3)C2)c1 ; "TFA"]

¹H NMR (400 MHz, METHANOL-d₄) δ ppm 7.49-7.57 (m, 3H) 7.39 (dd, J=8.16, 4.19 Hz, 3H) 7.11-7.29 (m, 3H) 6.90-6.97 (m, 1H) 6.83 (br. s., 2H) 6.74 (d, J=8.82 Hz, 1H) 3.73 (s, 3H) 3.37 (br. s., 3H) 3.09-3.26 (m, 2H) 3.00 (br. s., 2H) 2.08-2.26 (m, 1H) 1.60-2.05 (m, 3H) 1.23-1.50 (m, 3H) 0.82-1.02 (m, 1H)

LCMS (ESI+): m/z 468.2 (M+H)

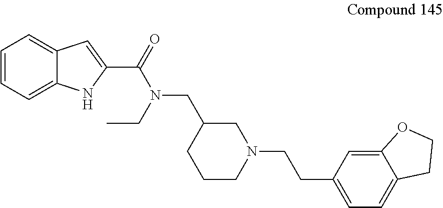 [SMILES: CCN(CC1CCCN(CCc2ccc3c(c2)OCC3)C1)C(=O)c1cc2ccccc2[nH]1]

¹H NMR (400 MHz, METHANOL-d₄) δ ppm 7.57-7.66 (m, 1H) 7.38-7.47 (m, 1H) 7.21 (t, J=7.50 Hz, 1H) 7.02-7.11 (m, 2H) 6.87 (br. s., 1H) 6.62-6.74 (m, 2H) 4.48 (t, J=8.60 Hz, 2H) 3.34-3.83 (m, 7H) 2.70-3.16 (m, 7H) 2.26-2.47 (m, 1H) 1.72-2.05 (m, 3H) 1.23-1.41 (m, 4H)

LCMS (ESI+): m/z 432.2 (M+H)

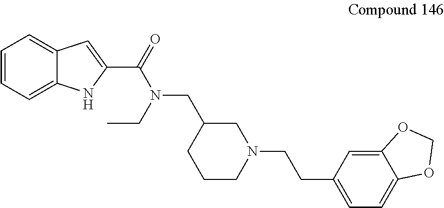 [SMILES: CCN(CC1CCCN(CCc2ccc3c(c2)OCO3)C1)C(=O)c1cc2ccccc2[nH]1]

¹H NMR (400 MHz, METHANOL-d₄) δ ppm 7.62 (d, J=7.94 Hz, 1H) 7.42 (d, J=8.38 Hz, 1H) 7.21 (t, J=7.50 Hz, 1H) 7.06 (t, J=7.50 Hz, 1H) 6.89 (br. s., 1H) 6.77 (s, 1H) 6.68-6.74 (m, 2H) 5.88 (s, 2H) 3.39-3.92 (m, 7H) 3.16-3.25 (m, 1H) 2.67-3.04 (m, 4H) 2.35 (br. s., 1H) 1.74-2.07 (m, 3H) 1.34 (br. s., 4H)

LCMS (ESI+): m/z 434.2 (M+H)

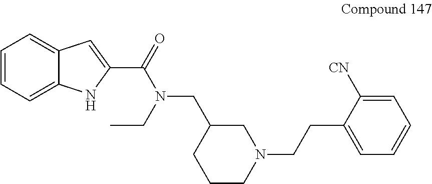

¹H NMR (400 MHz, METHANOL-d₄) δ ppm 7.72-7.84 (m, 1H) 7.61-7.69 (m, 2H) 7.50-7.59 (m, 1H) 7.39-7.49 (m, 2H) 7.20-7.28 (m, 1H) 7.02-7.12 (m, 1H) 6.90 (s, 1H) 3.81 (br. s., 1H) 3.59-3.74 (m, 2H) 3.40 (d, J=6.62 Hz, 3H) 3.34 (br. s., 3H) 3.10-3.27 (m, 1H) 2.83-3.07 (m, 1H) 2.08 (d, J=15.00 Hz, 1H) 1.96 (br. s., 1H) 1.77-1.87 (m, 1H) 1.42-1.49 (m, 1H) 1.36 (t, J=6.84 Hz, 3H)

LCMS (ESI+): m/z 415.2 (M+H)

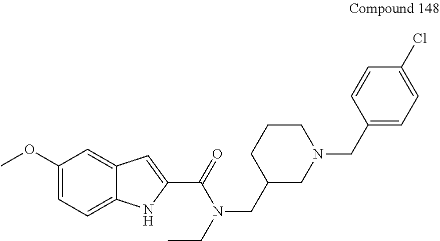 [SMILES: CCN(CC1CCCN(Cc2ccc(Cl)cc2)C1)C(=O)c1cc2cc(OC)ccc2[nH]1]

¹H NMR (400 MHz, METHANOL-d₄) δ ppm 7.28-7.58 (m, 5H) 7.08 (d, J=1.76 Hz, 1H) 6.85-6.95 (m, 1H) 6.72 (s, 1H) 4.21-4.34 (m, 2H) 3.80 (s, 3H) 3.68 (dd, J=13.67, 7.06 Hz, 2H) 3.34-3.50 (m, 3H) 2.98-3.17 (m, 1H) 2.69-2.95 (m, 2H) 2.28 (br. s., 1H) 1.66-2.08 (m, 4H) 1.24-1.38 (m, 3H)

LCMS (ESI+): m/z 440.1 (M+H)

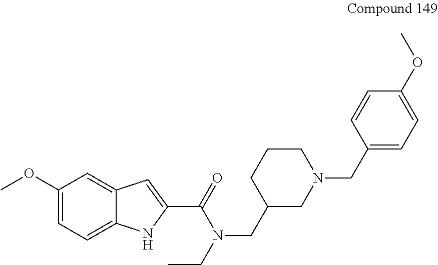 [SMILES: CCN(CC1CCCN(Cc2ccc(OC)cc2)C1)C(=O)c1cc2cc(OC)ccc2[nH]1]

¹H NMR (400 MHz, METHANOL-d₄) δ ppm 7.28-7.45 (m, 3H) 7.05-7.12 (m, 1H) 6.86-6.95 (m, 2H) 6.75-6.80 (m, 1H) 6.70 (s, 1H) 4.13-4.27 (m, 2H) 3.78-3.84 (m, 3H) 3.68 (s, 4H) 3.32-3.52 (m, 4H) 2.98-3.12 (m, 1H) 2.87 (s, 1H) 2.72 (br. s., 1H) 2.26 (br. s., 1H) 1.65-2.09 (m, 4H) 1.24-1.36 (m, 3H)

LCMS (ESI+): m/z 436.2 (M+H)

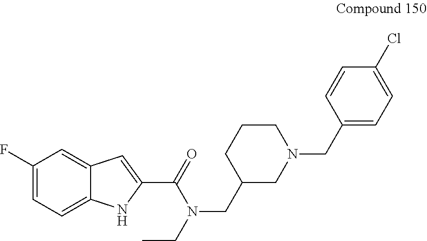 [SMILES: CCN(CC1CCCN(Cc2ccc(Cl)cc2)C1)C(=O)c1cc2cc(F)ccc2[nH]1]

¹H NMR (400 MHz, METHANOL-d₄) δ ppm 7.22-7.60 (m, 6H) 7.00 (t, J=9.04 Hz, 1H) 6.73-6.86 (m, 1H) 4.21-4.37 (m, 2H) 3.60-3.83 (m, 3H) 3.36-3.48 (m, 2H) 2.67-3.14 (m, 3H) 2.30 (br. s., 1H) 1.86-2.04 (m, 2H) 1.75 (d, J=13.67 Hz, 1H) 1.20-1.45 (m, 4H)

LCMS (ESI+): m/z 428.1 (M+H)

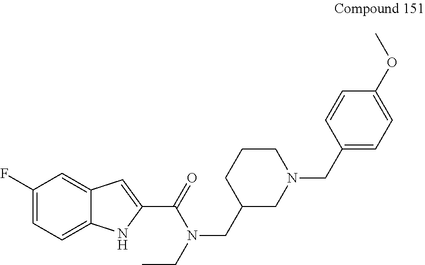 [SMILES: CCN(CC1CCCN(Cc2ccc(OC)cc2)C1)C(=O)c1cc2cc(F)ccc2[nH]1]

¹H NMR (400 MHz, METHANOL-d₄) δ ppm 7.22-7.42 (m, 3H) 6.95-7.11 (m, 2H) 6.83 (br. s., 2H) 6.72 (s, 1H) 3.48-3.93 (m, 10H) 3.11 (q, J=7.20 Hz, 3H) 2.19 (br. s., 1H) 1.60-1.88 (m, 3H) 1.21-1.33 (m, 4H)

LCMS (ESI+): m/z 424.2 (M+H)

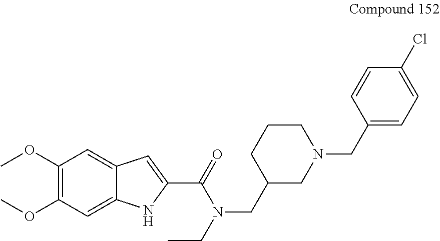 [SMILES: CCN(CC1CCCN(Cc2ccc(Cl)cc2)C1)C(=O)c1cc2cc(OC)c(OC)cc2[nH]1]

¹H NMR (400 MHz, METHANOL-d₄) δ ppm 7.25 (br. s., 4H) 7.11 (s, 1H) 6.96 (br. s., 1H) 6.68 (s, 1H) 3.87 (d, J=5.26 Hz, 8H) 3.41-3.75 (m, 5H) 2.78 (br. s., 2H) 2.00-2.15 (m, 2H) 1.52-1.88 (m, 3H) 1.24 (t, J=6.58 Hz, 3H) 1.07 (d, J=10.09 Hz, 1H)

LCMS (ESI+): m/z 470.1 (M+H)

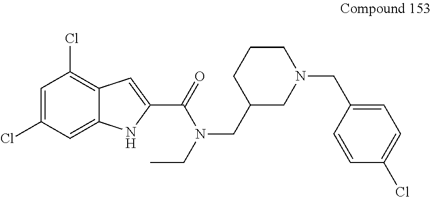 [SMILES: CCN(CC1CCCN(Cc2ccc(Cl)cc2)C1)C(=O)c1cc2c(Cl)cc(Cl)cc2[nH]1]

¹H NMR (400 MHz, METHANOL-d₄) δ ppm 7.42-7.58 (m, 4H) 7.31 (d, J=7.94 Hz, 1H) 7.13-7.20 (m, 1H) 6.81-6.88 (m, 1H) 4.25-4.36 (m, 2H) 3.62-3.82 (m, 3H) 3.36-3.51 (m, 3H) 2.76-2.98 (m, 2H) 2.32 (br. s., 1H) 1.90-2.04 (m, 2H) 1.76 (d, J=14.55 Hz, 1H) 1.25-1.39 (m, 4H)

LCMS (ESI+): m/z 479.1 (M+H)

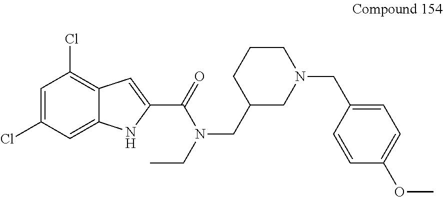 [SMILES: CCN(CC1CCCN(Cc2ccc(OC)cc2)C1)C(=O)c1cc2c(Cl)cc(Cl)cc2[nH]1]

¹H NMR (400 MHz, METHANOL-d₄) δ ppm 7.04-7.46 (m, 4H) 6.79 (br. s., 3H) 3.37-3.81 (m, 10H) 2.54-2.97 (m, 2H) 1.88-2.19 (m, 2H) 1.54-1.80 (m, 3H) 1.24 (br. s., 3H) 1.08 (br. s., 1H)

LCMS (ESI+): m/z 474.1 (M+H)

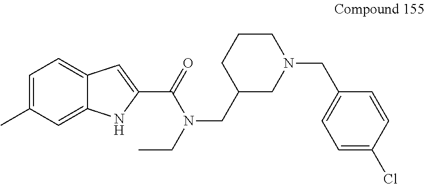 [SMILES: CCN(CC1CCCN(Cc2ccc(Cl)cc2)C1)C(=O)c1cc2ccc(C)cc2[nH]1]

¹H NMR (400 MHz, METHANOL-d₄) δ ppm 7.47-7.59 (m, 2H), 7.44 (br s, 2H), 7.28-7.37 (m, 1H), 7.25 (br s, 1H), 6.93 (br d, J=7.3 Hz, 1H), 6.76 (br s, 1H), 4.28 (br s, 2H), 3.61-3.87 (m, 3H), 3.38-3.51 (m, 2H), 2.74-2.97 (m, 2H), 2.44 (br s, 3H), 2.30 (br s, 1H), 1.87-2.06 (m, 3H), 1.75 (br d, J=13.7 Hz, 1H), 1.36 (br s, 1H), 1.29 (br s, 3H)

LCMS (ESI+): m/z 424.1 (M+H)

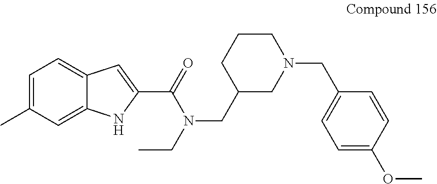

¹H NMR (400 MHz, METHANOL-d₄) δ ppm 7.49 (br d, J=7.5 Hz, 1H), 7.25 (br d, J=17.6 Hz, 3H), 6.93 (br d, J=8.8 Hz, 1H), 6.84 (br d, J=6.8 Hz, 2H), 6.73 (br s, 1H), 3.59-4.07 (m, 8H), 3.39 (br s, 1H), 2.91-3.21 (m, 3H), 2.44 (s, 3H), 2.22 (br s, 1H), 1.85 (br s, 2H), 1.70 (br s, 2H), 1.19-1.33 (m, 4H)

LCMS (ESI+): m/z 420.2 (M+H)

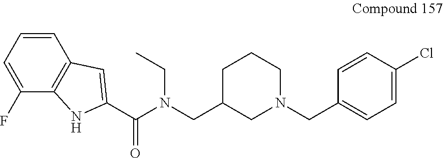 [SMILES: CCN(CC1CCCN(Cc2ccc(Cl)cc2)C1)C(=O)c1cc2cccc(F)c2[nH]1]

¹H NMR (400 MHz, METHANOL-d₄) δ ppm 7.54 (br s, 1H), 7.47 (br s, 3H), 7.34 (br s, 1H), 6.89-7.05 (m, 2H), 6.82 (br s, 1H), 4.30 (br s, 2H), 3.66 (br s, 3H), 3.44 (br d, J=16.8 Hz, 2H), 2.77-3.00 (m, 3H), 2.29 (br s, 1H), 2.03 (br d, J=15.0 Hz, 1H), 1.92 (br s, 1H), 1.76 (br s, 1H), 1.26-1.35 (m, 4H)

LCMS (ESI+): m/z 428.1 (M+H)

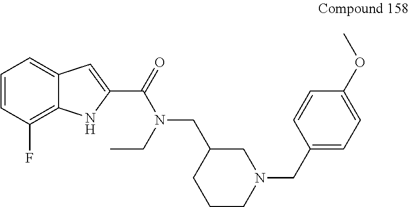 [SMILES: CCN(CC1CCCN(Cc2ccc(OC)cc2)C1)C(=O)c1cc2cccc(F)c2[nH]1]

¹H NMR (400 MHz, METHANOL-d₄) δ ppm 7.41 (d, J=7.9 Hz, 1H), 7.21 (br s, 1H), 6.86-7.05 (m, 3H), 6.79 (br s, 3H), 3.71 (s, 4H), 3.32-3.68 (m, 7H), 1.84-2.19 (m, 2H), 1.51-1.83 (m, 3H), 1.17-1.31 (m, 5H)

LCMS (ESI+): m/z 424.1 (M+H)

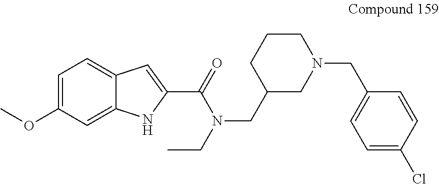 [SMILES: CCN(CC1CCCN(Cc2ccc(Cl)cc2)C1)C(=O)c1cc2ccc(OC)cc2[nH]1]

¹H NMR (400 MHz, METHANOL-d₄) δ ppm 7.30-7.61 (m, 5H) 6.94-7.02 (m, 1H) 6.72-6.87 (m, 2H) 4.24-4.36 (m, 2H) 3.62-3.95 (m, 6H) 3.37-3.48 (m, 2H) 3.00-3.16 (m, 1H) 2.72-2.95 (m, 2H) 2.31 (br. s., 1H) 1.87-2.05 (m, 2H) 1.70-1.83 (m, 1H) 1.21-1.39 (m, 4H)

LCMS (ESI+): m/z 440.1 (M+H)

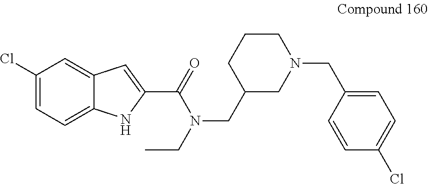 [SMILES: CCN(CC1CCCN(Cc2ccc(Cl)cc2)C1)C(=O)c1cc2cc(Cl)ccc2[nH]1]

¹H NMR (400 MHz, METHANOL-d₄) δ ppm 7.62-7.68 (m, 1H) 7.32-7.58 (m, 5H) 7.18-7.27 (m, 1H) 6.76-6.87 (m, 1H) 4.23-4.36 (m, 2H) 3.61-3.92 (m, 3H) 3.36-3.50 (m, 2H) 3.03-3.16 (m, 1H) 2.73-2.96 (m, 2H) 2.29 (br. s., 1H) 1.88-2.06 (m, 2H) 1.75 (d, J=12.79 Hz, 1H) 1.21-1.39 (m, 4H)

LCMS (ESI+): m/z 444.0 (M+H)

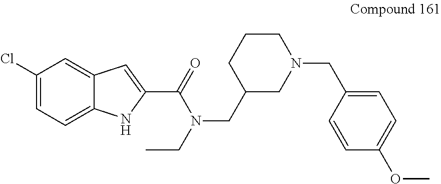 [SMILES: CCN(CC1CCCN(Cc2ccc(OC)cc2)C1)C(=O)c1cc2cc(Cl)ccc2[nH]1]

¹H NMR (400 MHz, METHANOL-d₄) δ ppm 7.61-7.68 (m, 1H) 7.31-7.50 (m, 3H) 7.18-7.26 (m, 1H) 6.94 (d, J=8.38 Hz, 1H) 6.78-6.85 (m, 1H) 6.75 (s, 1H) 4.13-4.29 (m, 2H) 3.34-3.81 (m, 8H) 2.99-3.13 (m, 1H) 2.68-2.93 (m, 2H) 2.27 (br. s., 1H) 1.86-2.05 (m, 2H) 1.75 (d, J=14.11 Hz, 1H) 1.20-1.37 (m, 4H)

LCMS (ESI+): m/z 440.0 (M+H)

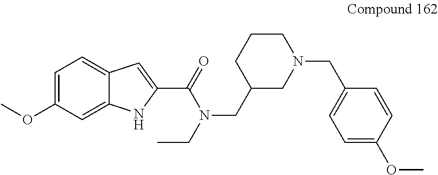

¹H NMR (400 MHz, METHANOL-d₄) δ ppm 7.47 (d, J=8.77 Hz, 1H) 7.19 (br. s., 2H) 6.91 (br. s., 1H) 6.80 (d, J=7.89 Hz, 2H) 6.70-6.76 (m, 2H) 3.80-3.84 (m, 3H) 3.50-3.75 (m, 8H) 3.43 (br. s., 1H) 2.87 (br. s., 1H) 2.16 (s, 2H) 1.90-2.05 (m, 1H) 1.76 (br. s., 2H) 1.60 (br. s., 1H) 1.26 (d, J=7.45 Hz, 4H) 1.11 (br. s., 1H)

LCMS (ESI+): m/z 436.1 (M+H)

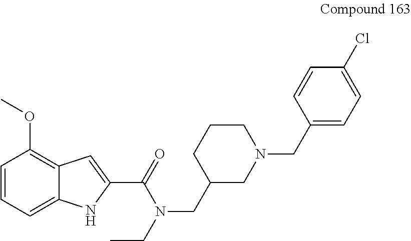 [SMILES: CCN(CC1CCCN(Cc2ccc(Cl)cc2)C1)C(=O)c1cc2c(OC)cccc2[nH]1]

¹H NMR (400 MHz, METHANOL-d₄) δ ppm 7.55 (br s, 1H) 7.44 (br s, 2H) 7.32 (br s, 1H) 7.16 (br s, 1H) 7.05 (br s, 1H) 6.87 (br s, 1H) 6.54 (br s, 1H) 4.29 (br s, 2H) 3.94 (br s, 3H) 3.61-3.72 (m, 2H) 3.39 (br s, 2H) 3.11 (br s, 2H) 2.72-2.98 (m, 2H) 2.54 (s, 1H) 2.30 (br s, 1H) 2.04 (br s, 1H) 1.92 (br s, 1H) 1.75 (br s, 1H) 1.26-1.42 (m, 3H)

LCMS (ESI+): m/z 440.1 (M+H)

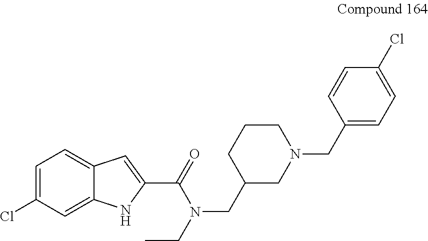 [SMILES: CCN(CC1CCCN(Cc2ccc(Cl)cc2)C1)C(=O)c1cc2ccc(Cl)cc2[nH]1]

¹H NMR (400 MHz, METHANOL-d₄) δ ppm 7.61 (br d, J=9.04 Hz, 1H) 7.53 (br d, J=10.80 Hz, 1H) 7.45 (br s, 3H) 7.35 (br s, 1H) 7.07 (br d, J=7.28 Hz, 1H) 6.82 (s, 1H) 4.29 (br s, 2H) 3.69 (br s, 2H) 3.40-3.51 (m, 2H) 3.09 (br d, J=11.25 Hz, 2H) 2.92 (br s, 1H) 2.80 (br s, 1H) 2.52 (s, 1H) 2.29 (br s, 1H) 2.02 (br d, J=17.64 Hz, 1H) 1.93 (br d, J=14.11 Hz, 1H) 1.73 (br s, 1H) 1.30 (br s, 3H)

LCMS (ESI+): m/z 444.1 (M+H)

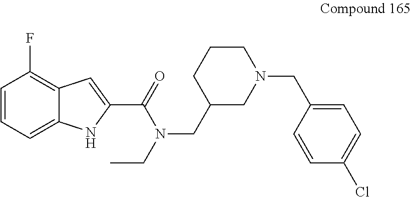 [SMILES: CCN(CC1CCCN(Cc2ccc(Cl)cc2)C1)C(=O)c1cc2c(F)cccc2[nH]1]

¹H NMR (400 MHz, METHANOL-d₄) δ ppm 7.41-7.56 (m, 3H) 7.13-7.35 (m, 3H) 6.83-6.90 (m, 1H) 6.75 (dd, J=10.36, 7.72 Hz, 1H) 4.23-4.35 (m, 2H) 3.67 (d, J=14.11 Hz, 2H) 3.34-3.49 (m, 3H) 3.00-3.15 (m, 1H) 2.73-2.95 (m, 2H) 2.28 (br. s., 1H) 1.86-2.04 (m, 2H) 1.73 (d, J=12.79 Hz, 1H) 1.22-1.39 (m, 4H)

LCMS (ESI+): m/z 428.1 (M+H)

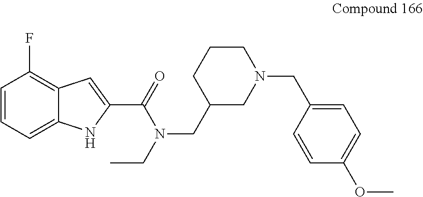 [SMILES: CCN(CC1CCCN(Cc2ccc(OC)cc2)C1)C(=O)c1cc2c(F)cccc2[nH]1]

¹H NMR (400 MHz, METHANOL-d₄) δ ppm 7.14-7.44 (m, 4H) 6.92 (d, J=8.38 Hz, 2H) 6.73-6.82 (m, 2H) 4.15-4.27 (m, 2H) 3.62-3.82 (m, 5H) 3.34-3.52 (m, 3H) 2.99-3.13 (m, 1H) 2.87 (t, J=11.91 Hz, 1H) 2.73 (br. s., 1H) 2.25 (br. s., 1H) 1.87-2.03 (m, 2H) 1.65-1.79 (m, 1H) 1.22-1.38 (m, 4H)

LCMS (ESI+): m/z 424.2 (M+H)

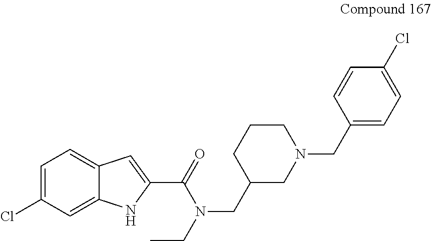 [SMILES: CCN(CC1CCCN(Cc2ccc(Cl)cc2)C1)C(=O)c1cc2ccc(Cl)cc2[nH]1]

¹H NMR (400 MHz, METHANOL-d₄) δ ppm 7.41 (s, 1H) 7.35 (br d, J=7.50 Hz, 1H) 7.17 (br t, J=8.27 Hz, 1H) 7.06 (br d, J=7.50 Hz, 1H) 6.92 (br d, J=9.04 Hz, 1H) 6.86 (s, 1H) 6.77 (br d, J=7.28 Hz, 1H) 6.54 (br d, J=7.06 Hz, 1H) 4.16-4.27 (m, 2H) 3.94 (s, 3H) 3.69 (br s, 3H) 3.49 (br s, 2H) 3.38 (br s, 2H) 3.04 (br s, 1H) 2.89 (br s, 1H) 2.74 (s, 1H) 2.51 (s, 1H) 2.25 (s, 1H) 2.02 (br d, J=16.76 Hz, 1H) 1.90 (br s, 1H) 1.72 (s, 2H) 1.27-1.36 (m, 3H)

LCMS (ESI+): m/z 436.1 (M+H)

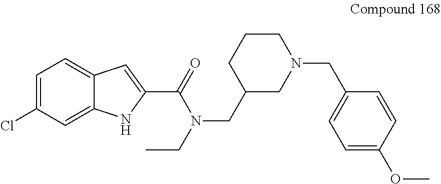 [SMILES: CCN(CC1CCCN(Cc2ccc(OC)cc2)C1)C(=O)c1cc2ccc(Cl)cc2[nH]1]

¹H NMR (400 MHz, METHANOL-d₄) δ ppm 7.57 (br d, J=8.60 Hz, 1H) 7.39 (br s, 1H) 7.16 (br s, 2H) 7.05 (br d, J=9.26 Hz, 1H) 6.76 (br d, J=16.10 Hz, 3H) 3.71 (br s, 6H) 3.38-3.51 (m, 3H) 2.80 (br s, 2H) 2.09 (br s, 2H) 1.84 (br s, 1H) 1.72 (br s, 2H) 1.59 (br s, 1H) 1.23 (br s, 3H) 1.03 (br s, 1H)

LCMS (ESI+): m/z 440.1 (M+H)

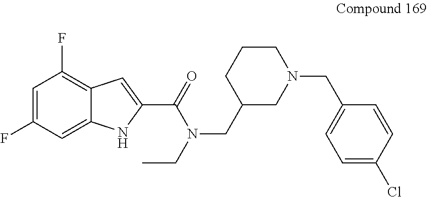 [SMILES: CCN(CC1CCCN(Cc2ccc(Cl)cc2)C1)C(=O)c1cc2c(F)cc(F)cc2[nH]1]

¹H NMR (400 MHz, METHANOL-d₄) δ ppm 7.45 (s, 4H), 7.02 (br d, J=9.0 Hz, 1H), 6.91-6.84 (m, 1H), 6.68 (br t, J=10.0 Hz, 1H), 4.30 (br s, 2H), 3.74-3.34 (m, 6H), 3.00-2.72 (m, 2H), 2.29 (br s, 1H), 2.05-1.90 (m, 2H), 1.30 (br s, 5H)

LCMS (ESI+): m/z 446.1 (M+H)

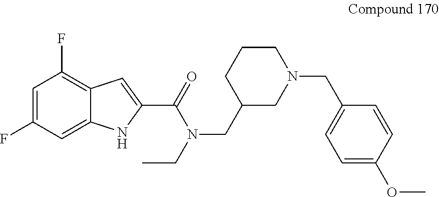 [SMILES: CCN(CC1CCCN(Cc2ccc(OC)cc2)C1)C(=O)c1cc2c(F)cc(F)cc2[nH]1]

¹H NMR (400 MHz, METHANOL-d₄) δ ppm 7.44-7.34 (m, 2H), 7.08-6.99 (m, 1H), 6.95 (br d, J=7.7 Hz, 2H), 6.83 (br d, J=0.9 Hz, 1H), 6.73-6.64 (m, 1H), 4.28-4.17 (m, 2H), 3.76-3.47 (m, 7H), 3.10-2.71 (m, 2H), 2.27 (br s, 1H), 2.08-1.62 (m, 4H), 1.44-1.19 (m, 5H)

LCMS (ESI+): m/z 442.1 (M+H)

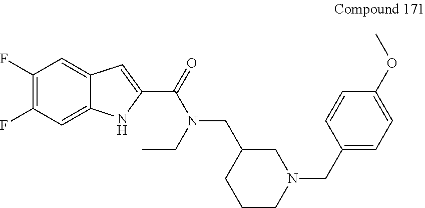 [SMILES: CCN(CC1CCCN(Cc2ccc(OC)cc2)C1)C(=O)c1cc2cc(F)c(F)cc2[nH]1]

¹H NMR (400 MHz, METHANOL-d₄) δ ppm 7.43-7.51 (m, 1H), 7.28-7.41 (m, 3H), 6.95 (d, J=8.3 Hz, 2H), 6.79-6.86 (m, 1H), 4.17-4.29 (m, 2H), 3.73 (s, 3H), 3.57-3.70 (m, 2H), 3.45 (br s, 1H), 3.36 (br d, J=13.2 Hz, 3H), 2.49-3.15 (m, 1H), 2.27 (s, 1H), 1.88-2.06 (m, 2H), 1.74 (d, J=13.2 Hz, 1H), 1.24-1.38 (m, 4H)

LCMS (ESI+): m/z 442.1 (M+H)

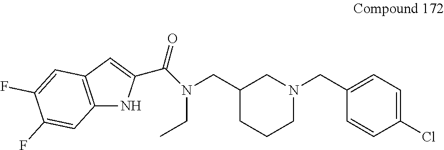 [SMILES: CCN(CC1CCCN(Cc2ccc(Cl)cc2)C1)C(=O)c1cc2cc(F)c(F)cc2[nH]1]

¹H NMR (400 MHz, METHANOL-d₄) δ ppm 7.54 (br d, J=7.7 Hz, 1H), 7.45 (s, 3H), 7.26-7.38 (m, 2H), 6.81 (s, 1H), 4.29 (d, J=5.7 Hz, 2H), 3.62-3.70 (m, 1H), 3.36-3.50 (m, 3H), 2.76-3.15 (m, 2H), 2.30 (br s, 1H), 1.88-2.06 (m, 3H), 1.66-1.83 (m, 1H), 1.24-1.40 (m, 5H)

LCMS (ESI+): m/z 446.2 (M+H)

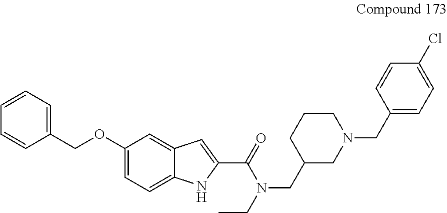 [SMILES: CCN(CC1CCCN(Cc2ccc(Cl)cc2)C1)C(=O)c1cc2cc(OCc3ccccc3)ccc2[nH]1]

¹H NMR (400 MHz, METHANOL-d₄) δ ppm 7.42-7.49 (m, 6H) 7.37-7.40 (m, 2H) 7.36 (s, 1H) 7.31 (br d, J=7.45 Hz, 2H) 7.15-7.20 (m, 1H) 7.01 (br d, J=9.21 Hz, 1H) 6.72 (s, 1H) 5.11 (s, 3H) 4.29 (br d, J=5.26 Hz, 2H) 3.35-3.49 (m, 4H) 2.73-2.98 (m, 2H) 2.29 (br s, 1H) 1.88-2.07 (m, 3H) 1.72 (br s, 1H) 1.26-1.39 (m, 5H)

LCMS (ESI+): m/z 516.2 (M+H)

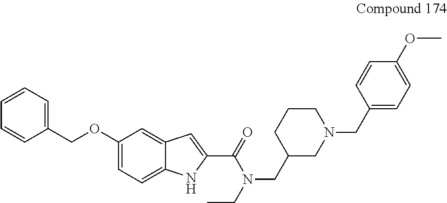 [SMILES: CCN(CC1CCCN(Cc2ccc(OC)cc2)C1)C(=O)c1cc2cc(OCc3ccccc3)ccc2[nH]1]

¹H NMR (400 MHz, METHANOL-d₄) δ ppm 7.47 (br d, J=7.94 Hz, 2H) 7.42 (s, 1H) 7.34-7.39 (m, 4H) 7.15-7.20 (m, 1H) 6.98-7.03 (m, 1H) 6.93 (d, J=8.60 Hz, 2H) 6.76-6.80 (m, 1H) 6.71 (s, 1H) 5.10-5.12 (m, 1H) 5.10-5.12 (m, 1H) 3.68 (s, 3H) 3.47 (br s, 3H) 3.31-3.35 (m, 2H) 2.80-3.09 (m, 2H) 2.25 (br s, 1H) 1.84-2.10 (m, 3H) 1.74 (br d, J=12.57 Hz, 1H) 1.23-1.41 (m, 5H)

LCMS (ESI+): m/z 512.3 (M+H)

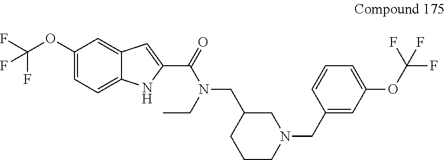 [SMILES: CCN(CC1CCCN(Cc2cccc(OC(F)(F)F)c2)C1)C(=O)c1cc2cc(OC(F)(F)F)ccc2[nH]1]

¹H NMR (400 MHz, METHANOL-d₄) δ ppm 7.54-7.43 (m, 5H) 7.40-7.31 (m, 1H) 7.12 (br d, J=8.6 Hz, 1H) 6.90-6.82 (m, 1H) 4.34 (s, 2H) 3.65 (dt, J=7.4, 14.5 Hz, 3H) 3.41 (br d, J=10.4 Hz, 3H) 2.98-2.77 (m, 2H) 2.54-2.26 (m, 1H) 2.03-1.87 (m, 2H) 1.81-1.66 (m, 1H) 1.36-1.22 (m, 4H)

LCMS (ESI+): m/z 544.1 (M+H)

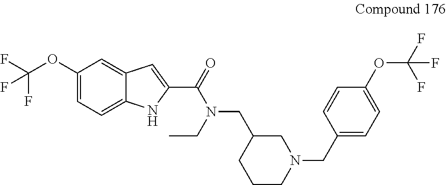 [SMILES: CCN(CC1CCCN(Cc2ccc(OC(F)(F)F)cc2)C1)C(=O)c1cc2cc(OC(F)(F)F)ccc2[nH]1]

¹H NMR (400 MHz, METHANOL-d₄) δ ppm 7.71-7.47 (m, 4H) 7.33 (br d, J=7.9 Hz, 1H) 7.22 (br d, J=6.8 Hz, 1H) 7.15 (br d, J=8.8 Hz, 1H) 6.95-6.82 (m, 1H) 4.39-4.31 (m, 2H) 3.86-3.62 (m, 3H) 3.52-3.37 (m, 3H) 3.22-3.01 (m, 1H) 2.96-2.77 (m, 1H) 2.57-2.27 (m, 1H) 2.06-1.87 (m, 2H) 1.84-1.68 (m, 1H) 1.39-1.24 (m, 4H)

LCMS (ESI+): m/z 544.1 (M+H)

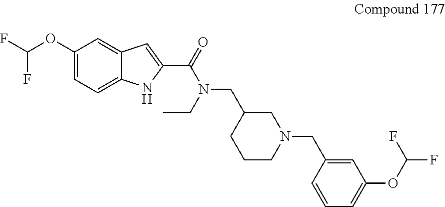 [SMILES: CCN(CC1CCCN(Cc2cccc(OC(F)F)c2)C1)C(=O)c1cc2cc(OC(F)F)ccc2[nH]1]

¹H NMR (400 MHz, METHANOL-d₄) δ ppm 7.38-7.50 (m, 3H) 7.28-7.38 (m, 2H) 7.17-7.28 (m, 1H) 7.00-7.15 (m, 1H) 6.79-6.93 (m, 2H) 6.52-6.76 (m, 1H) 4.27-4.39 (m, 2H) 3.61-3.84 (m, 3H) 3.45 (br t, J=12.94 Hz, 3H) 2.69-3.02 (m, 1H) 2.33 (br s, 1H) 1.87-2.06 (m, 2H) 1.69-1.83 (m, 1H) 1.26-1.37 (m, 4H)

LCMS (ESI+): m/z 508.1 (M+H)

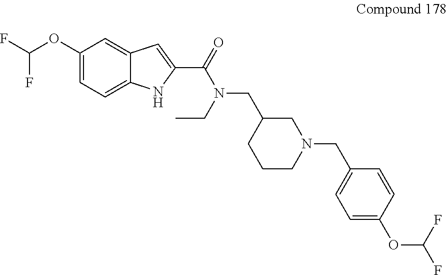 [SMILES: CCN(CC1CCCN(Cc2ccc(OC(F)F)cc2)C1)C(=O)c1cc2cc(OC(F)F)ccc2[nH]1]

¹H NMR (400 MHz, METHANOL-d₄) δ ppm 7.39-7.55 (m, 3H) 7.14-7.24 (m, 2H) 7.08 (br d, J=8.77 Hz, 2H) 6.77-6.94 (m, 2H) 6.52-6.74 (m, 1H) 4.26-4.36 (m, 2H) 3.58-3.84 (m, 3H) 3.36-3.56 (m, 3H) 2.70-2.99 (m, 1H) 2.32 (br s, 1H) 1.87-2.07 (m, 2H) 1.65-1.84 (m, 1H) 1.23-1.41 (m, 4H)

LCMS (ESI+): m/z 508.1 (M+H)

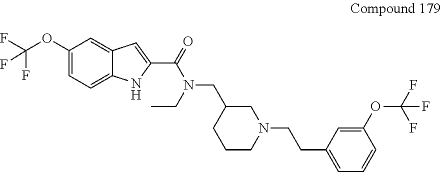 [SMILES: CCN(CC1CCCN(CCc2cccc(OC(F)(F)F)c2)C1)C(=O)c1cc2cc(OC(F)(F)F)ccc2[nH]1]

¹H NMR (400 MHz, METHANOL-d₄) δ ppm 7.55 (s, 1H) 7.49 (br d, J=8.8 Hz, 1H) 7.45-7.40 (m, 1H) 7.29 (br d, J=7.5 Hz, 1H) 7.24 (br s, 1H) 7.18 (br d, J=8.8 Hz, 1H) 7.15-7.12 (m, 1H) 6.94 (br s, 1H) 3.78 (br s, 2H) 3.66 (br d, J=12.3 Hz, 2H) 3.59 (br d, J=11.0 Hz, 1H) 3.40-3.33 (m, 3H) 3.11 (br d, J=8.8 Hz, 2H) 2.99-2.79 (m, 2H) 2.53-2.34 (m, 1H) 2.05 (br d, J=13.2 Hz, 1H) 1.96 (br d, J=11.4 Hz, 1H) 1.88-1.76 (m, 1H) 1.41 (br s, 1H) 1.34 (br t, J=6.8 Hz, 3H)

LCMS (ESI+): m/z 558.1 (M+H)

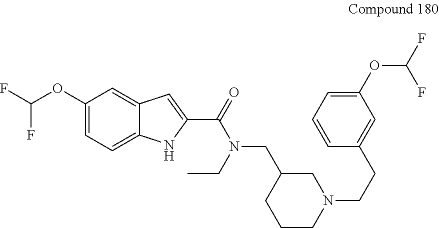 [SMILES: CCN(CC1CCCN(CCc2cccc(OC(F)F)c2)C1)C(=O)c1cc2cc(OC(F)F)ccc2[nH]1]

¹H NMR (400 MHz, METHANOL-d₄) δ ppm 7.39-7.47 (m, 2H) 7.29 (s, 1H) 6.93-7.11 (m, 4H) 6.76-6.92 (m, 2H) 6.50-6.75 (m, 1H) 3.41-3.87 (m, 5H) 2.72-3.01 (m, 4H) 2.12-2.33 (m, 2H) 1.55-1.87 (m, 4H) 1.30 (br s, 5H)

LCMS (ESI+): m/z 522.2 (M+H)

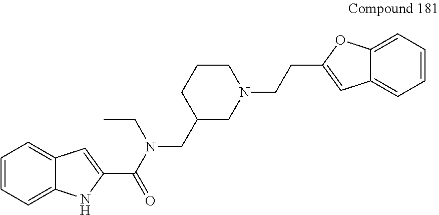 [SMILES: CCN(CC1CCCN(CCc2cc3ccccc3o2)C1)C(=O)c1cc2ccccc2[nH]1]

¹H NMR (400 MHz, CHLOROFORM-d) δ ppm 7.67 (br d, J=8.16 Hz, 1H) 7.35-7.48 (m, 3H) 7.11-7.23 (m, 3H) 6.85 (br d, J=18.08 Hz, 1H) 6.40 (br s, 1H) 3.78 (br s, 2H) 3.40 (br d, J=14.33 Hz, 2H) 3.00 (br d, J=7.06 Hz, 2H) 2.85 (br d, J=5.95 Hz, 2H) 2.21 (br s, 2H) 1.29-1.41 (m, 4H)

LCMS (ESI+): m/z 430.1 (M+H)

### Example 5: General Protocol B for Synthesis of Exemplary Compounds

General Protocol B to synthesize exemplary compounds of Formula (I) is described in Scheme 2 and the procedures set forth below.

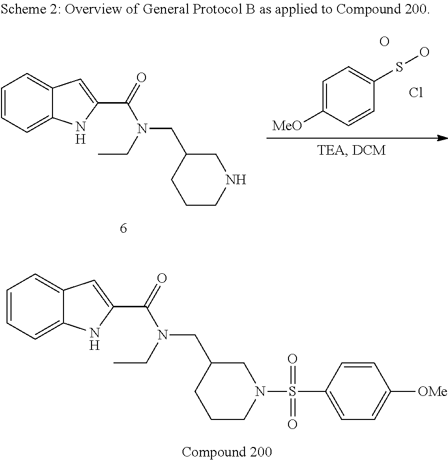

A mixture of compound 6 (30.0 mg, 93.2 μmol, 1.0 eq, HCl salt) and TEA (47.2 mg, 466.1 μmol, 5.0 eq) in DCM (1 mL) was added 4-methoxybenzenesulfonyl chloride (21.2 mg, 102.5 μmol, 1.1 eq) at −10° C., and then the mixture was stirred at 25° C. for 0.2 hour. The reaction was monitored by LCMS and allowed to run until completion. The reaction mixture was filtered and concentrated under reduced pressure to give a residue. The residue was purified by prep-HPLC (TFA condition) to give 6.4 mg of compound 200 (15.1% yield) as a white solid.

¹H NMR (400 MHz, CHLOROFORM-d) δ ppm 9.32 (br. s., 1H) 7.65 (br. s., 3H) 7.42 (br. s., 1H) 7.29 (br. s., 1H) 7.14 (br. s., 1H) 6.94 (br. s., 2H) 6.82 (br. s., 1H) 3.85 (br. s., 3H) 3.59-3.37 (m, 4H) 2.48 (br. s., 1H) 2.36 (br. s., 1H) 2.19 (br. s., 1H) 1.81-1.59 (m, 4H) 1.35 (br. s., 3H) 1.25 (br. s., 1H) 1.12 (br. s., 1H)

LCMS (ESI+): m/z 456.1 (M+H)

### Example 6: General Protocol C for Synthesis of Exemplary Compounds

General Protocol C to synthesize exemplary compounds of Formula (I) is described in Scheme 3 and the procedures set forth below.

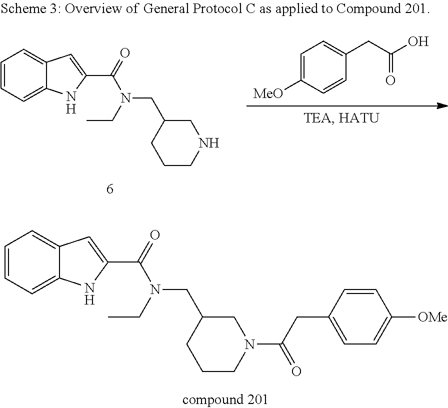

A mixture of compound 6 (30.0 mg, 93.2 μmol, 1.0 eq, HCl), HATU (42.5 mg, 111.9 μmol, 1.2 eq), Et₃N (18.9 mg, 186.4 μmol, 2.0 eq) in DMF (1 mL) was stirred at 15° C. for 10 min, then 2-(4-methoxyphenyl) acetic acid (15.5 mg, 93.2 μmol, 1.0 eq) was added, and then the mixture was stirred at 15° C. for 16 hrs. The reaction was monitored by LCMS and allowed to run until completion. The reaction mixture was filtered. The filtrate was purified by prep-HPLC (TFA condition) to give 23.5 mg of compound 201 (55.9% yield, 96.1% purity) as a white solid.

¹H NMR (400 MHz, METHANOL-d₄) δ ppm 7.40-7.70 (m, 1H) 7.00-7.27 (m, 5H) 6.86 (d, J=6.62 Hz, 2H) 6.58 (br. s., 1H) 4.30-4.55 (m, 1H) 3.72-3.80 (m, 4H) 3.51-3.64 (m, 5H) 2.51-3.21 (m, 2H) 1.77 (d, J=11.03 Hz, 2H) 1.48 (br. s., 2H) 1.15-1.37 (m, 6H)

LCMS (ESI+): m/z 434.2 (M+H)

### Example 7: General Protocol D for Synthesis of Exemplary Compounds

General Protocol D to synthesize exemplary compounds of Formula (I) is described in Scheme 4 and the procedures set forth below.

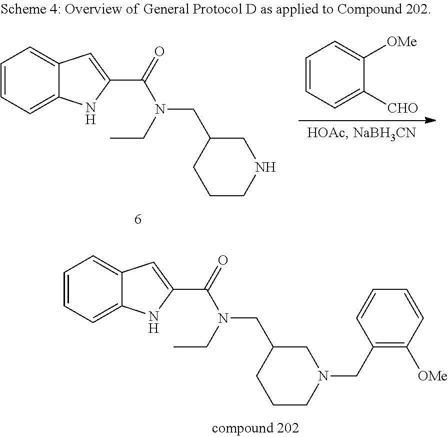

A mixture of compound 6 (30.0 mg, 105.1 μmol, 1.0 eq), 2-methoxybenzaldehyde (21.5 mg, 157.7 μmol, 1.5 eq), HOAc (631 μg, 10.5 μmol, 0.1 eq) in MeOH (1.5 mL) was stirred for 1 hour at 0° C., then NaBH₃CN (13.2 mg, 210 μmol, 2.0 eq) was added at the same temperature. The reaction was allowed to warm to 20° C. and stirred for 3 hours under N₂ atmosphere. The reaction was monitored by LCMS and allowed to run until completion. It was filtered. The filtrate was purified by prep-HPLC (TFA condition) to give 58 mg of compound 202 (99.0% yield, 93.5% purity, TFA salt) as a white solid.

¹H NMR (400 MHz, METHANOL-d₄) δ ppm 7.63 (d, J=7.94 Hz, 1H) 7.35-7.48 (m, 3H) 7.24 (t, J=7.79 Hz, 1H) 6.99-7.12 (m, 3H) 6.80 (s, 2H) 4.24-4.40 (m, 1H) 4.24-4.40 (m, 1H) 3.83-3.90 (m, 3H) 3.63-3.75 (m, 2H) 3.38-3.53 (m, 3H) 3.13 (br s, 1H) 2.97 (br t, J=12.02 Hz, 1H) 2.72-2.90 (m, 1H) 2.34 (br s, 1H) 1.88-2.05 (m, 2H) 1.72-1.85 (m, 1H) 1.25-1.39 (m, 4H)

LCMS (ESI+): m/z 406.1 (M+H)

The following compounds were prepared according to General Protocols B-D:

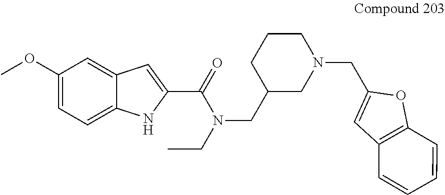 [SMILES: CCN(CC1CCCN(Cc2cc3ccccc3o2)C1)C(=O)c1cc2cc(OC)ccc2[nH]1]

¹H NMR (400 MHz, METHANOL-d₄) δ ppm 7.59 (br s, 1H) 7.45 (br d, J=8.33 Hz, 1H) 7.33 (br d, J=9.21 Hz, 2H) 7.24 (br s, 1H) 7.11 (br s, 1H) 7.06 (d, J=1.75 Hz, 1H) 6.91 (dd, J=9.21, 2.19 Hz, 1H) 6.69 (s, 1H) 4.55 (br s, 2H) 3.83 (s, 4H) 3.63-3.76 (m, 1H) 3.43 (br s, 3H) 2.38 (br s, 1H) 2.04 (br s, 2H) 1.87-1.98 (m, 2H) 1.31 (br t, J=6.80 Hz, 5H)

LCMS (ESI+): m/z 446.1 (M+H)

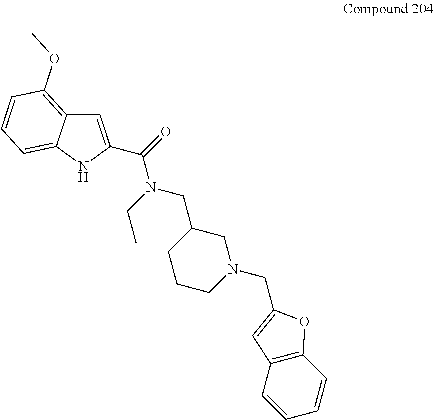 [SMILES: CCN(CC1CCCN(Cc2cc3ccccc3o2)C1)C(=O)c1cc2c(OC)cccc2[nH]1]

¹H NMR (400 MHz, METHANOL-d₄) δ ppm 7.60 (br d, J=7.06 Hz, 1H) 7.44 (br d, J=7.94 Hz, 1H) 7.21-7.33 (m, 2H) 7.17 (t, J=7.94 Hz, 1H) 7.10 (br s, 1H) 7.04 (br d, J=7.94 Hz, 1H) 6.85 (s, 1H) 6.54 (d, J=7.72 Hz, 1H) 6.51-6.56 (m, 1H) 4.56 (br s, 2H) 3.93 (s, 3H) 3.78 (br s, 1H) 3.70 (br s, 2H) 3.52 (br d, J=14.55 Hz, 1H) 3.47 (br s, 1H) 3.12-3.23 (m, 1H) 2.82-3.06 (m, 2H) 2.34 (br s, 1H) 2.05 (br d, J=14.33 Hz, 1H) 1.92 (br d, J=12.79 Hz, 1H) 1.80 (br s, 1H) 1.30 (br s, 4H)

LCMS (ESI+): m/z 446.1 (M+H)

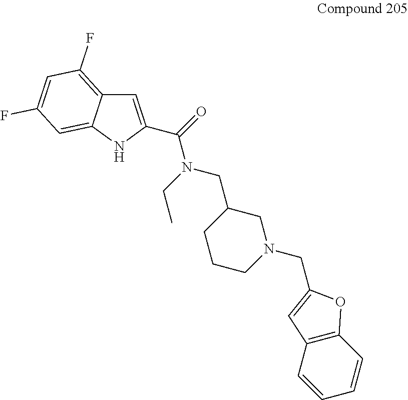 [SMILES: CCN(CC1CCCN(Cc2cc3ccccc3o2)C1)C(=O)c1cc2c(F)cc(F)cc2[nH]1]

¹H NMR (400 MHz, METHANOL-d₄) δ ppm 7.60 (br d, J=7.72 Hz, 1H) 7.46 (br d, J=8.38 Hz, 1H) 7.20-7.34 (m, 2H) 7.11 (s, 1H) 7.00 (br d, J=9.26 Hz, 1H) 6.78 (s, 1H) 6.68 (td, J=10.25, 1.98 Hz, 1H) 4.56 (br d, J=5.73 Hz, 2H) 3.64-3.76 (m, 2H) 3.58 (br d, J=12.13 Hz, 1H) 3.49 (br d, J=13.23 Hz, 2H) 3.18 (br s, 1H) 2.84-3.07 (m, 2H) 2.34 (br s, 1H) 2.05 (br d, J=14.55 Hz, 1H) 1.92 (br d, J=12.35 Hz, 1H) 1.79 (br d, J=14.33 Hz, 1H) 1.28 (br t, J=6.95 Hz, 4H)

LCMS (ESI+): m/z 452.1 (M+H)

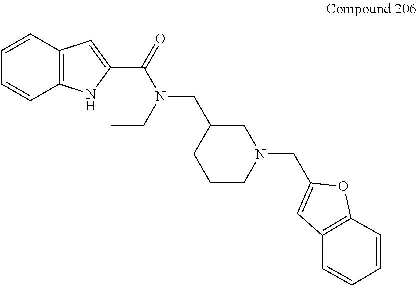 [SMILES: CCN(CC1CCCN(Cc2cc3ccccc3o2)C1)C(=O)c1cc2ccccc2[nH]1]

¹H NMR (400 MHz, METHANOL-d₄) δ ppm 7.54-7.65 (m, 2H) 7.44 (br d, J=4.63 Hz, 2H) 7.20-7.35 (m, 3H) 7.05-7.14 (m, 2H) 6.72-6.78 (m, 1H) 4.57 (br s, 2H) 3.65-3.86 (m, 3H) 3.43-3.64 (m, 3H) 2.82-3.12 (m, 2H) 2.36 (br s, 1H) 2.05 (br d, J=15.21 Hz, 1H) 1.93 (br d, J=13.01 Hz, 1H) 1.71-1.87 (m, 1H) 1.30 (br d, J=6.39 Hz, 5H)

LCMS (ESI+): m/z 416.1 (M+H)

### Example 8: General Protocol E for Synthesis of Exemplary Compounds

General Protocol E to synthesize exemplary compounds of Formula (I) is described in Scheme 5 and the procedures set forth below.

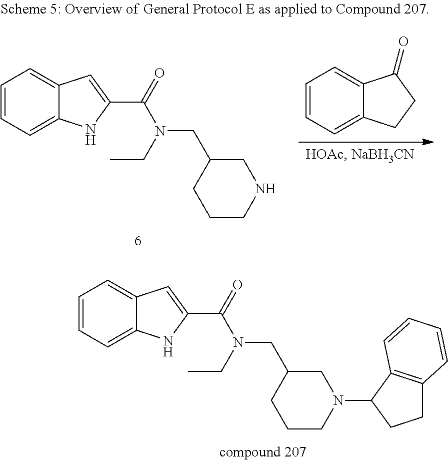

A mixture of compound 6 (50.0 mg, 175.2 μmol, 1.0 eq), indan-1-one (116 mg, 876 μmol, 105 μL, 5.0 eq), AcOH (1.1 mg, 17.5 μmol, 0.1 eq), NaBH₃CN (55 mg, 876 μmol, 5.0 eq) in MeOH (2 mL) was stirred at 80° C. for 12 hours. The reaction was monitored by LCMS and allowed to run until completion. The reaction mixture was filtered. The residue was purified by prep-HPLC (TFA condition) to give 8.8 mg of compound 207 (9.7% yield, TFA salt) as a pink solid.

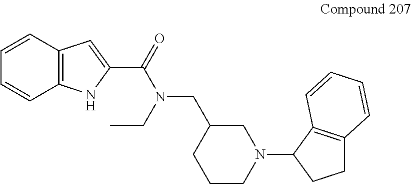 [SMILES: CCN(CC1CCCN(C2CCc3ccccc32)C1)C(=O)c1cc2ccccc2[nH]1]

¹H NMR (400 MHz, METHANOL-d₄) δ ppm 7.66-7.61 (m, 1H) 7.58-7.50 (m, 1H) 7.46 (d, J=8.4 Hz, 1H) 7.40-7.32 (m, 2H) 7.30-7.22 (m, 2H) 7.13-7.06 (m, 1H) 6.88-6.72 (m, 1H) 6.59-6.46 (m, 1H) 3.86-3.61 (m, 3H) 3.50-3.37 (m, 2H) 3.28-3.08 (m, 3H) 3.06-2.95 (m, 2H) 2.84 (br s, 1H) 2.55-2.45 (m, 2H) 2.00 (br d, J=11.5 Hz, 1H) 1.91 (br d, J=11.5 Hz, 1H) 1.83-1.69 (m, 1H) 1.37-1.24 (m, 4H)

LCMS (ESI+): m/z 402.1 (M+H)

The following compounds were prepared analogously:

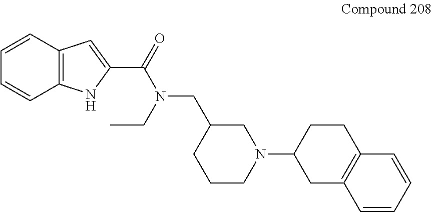 [SMILES: CCN(CC1CCCN(C2CCc3ccccc3C2)C1)C(=O)c1cc2ccccc2[nH]1]

The reaction mixture was stirred at 50° C. for 24 hrs.

¹H NMR (400 MHz, CHLOROFORM-d) δ ppm 7.68 (d, J=7.94 Hz, 1H) 7.39 (br s, 1H) 7.27-7.31 (m, 1H) 7.01-7.17 (m, 5H) 6.90 (br s, 1H) 3.82 (br s, 2H) 3.45 (br s, 1H) 2.78-2.95 (m, 7H) 2.05-2.30 (m, 2H) 1.61-1.85 (m, 7H) 1.27-1.46 (m, 4H)

LCMS (ESI+): m/z 416.3 (M+H)

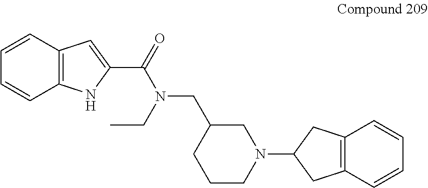 [SMILES: CCN(CC1CCCN(C2Cc3ccccc3C2)C1)C(=O)c1cc2ccccc2[nH]1]

The reaction mixture was stirred at 50° C. for 12 hrs

¹H NMR (400 MHz, METHANOL-d) δ ppm 7.65 (d, J=7.94 Hz, 1H) 7.45 (d, J=7.72 Hz, 1H) 7.16-7.28 (m, 5H) 7.05-7.12 (m, 1H) 6.87 (s, 1H) 4.08 (br t, J=7.72 Hz, 1H) 3.61-3.84 (m, 3H) 3.37-3.59 (m, 5H) 3.16-3.26 (m, 2H) 2.80-3.02 (m, 2H) 2.36 (br s, 1H) 2.05 (br d, J=14.55 Hz, 1H) 1.95 (br d, J=9.70 Hz, 1H) 1.80 (br d, J=14.77 Hz, 1H) 1.26-1.45 (m, 4H)

LCMS (ESI+): m/z 402.1 (M+H)

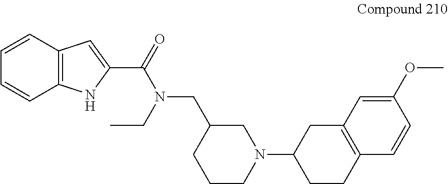 [SMILES: CCN(CC1CCCN(C2CCc3ccc(OC)cc3C2)C1)C(=O)c1cc2ccccc2[nH]1]

The reaction mixture was stirred at 50° C. for 12 hrs.

¹H NMR (400 MHz, METHANOL-d₄) δ ppm 7.62 (d, J=7.9 Hz, 1H) 7.42 (d, J=8.2 Hz, 1H) 7.23-7.18 (m, 1H) 7.09-7.04 (m, 1H) 6.93 (d, J=8.4 Hz, 1H) 6.84 (br s, 1H) 6.66-6.57 (m, 2H) 3.72 (d, J=2.9 Hz, 5H) 3.65-3.40 (m, 2H) 3.04-2.64 (m, 8H) 2.40-2.30 (m, 1H) 2.19-2.02 (m, 3H) 1.84-1.55 (m, 4H) 1.31 (br t, J=6.9 Hz, 3H)

LCMS (ESI+): m/z 396.1 (M+H)

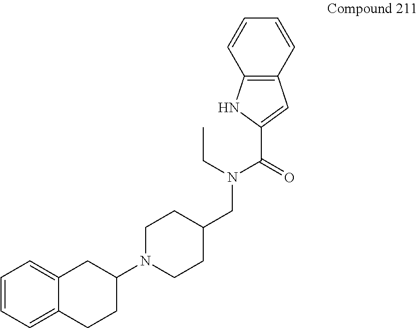 [SMILES: CCN(CC1CCN(C2CCc3ccccc3C2)CC1)C(=O)c1cc2ccccc2[nH]1]

The reaction mixture was stirred at 80° C. for 16 hrs.

¹H NMR (400 MHz, METHANOL-d₄) δ ppm 7.59 (d, J=7.94 Hz, 1H) 7.41 (d, J=8.38 Hz, 1H) 7.19 (td, J=7.72, 1.10 Hz, 1H) 7.00-7.06 (m, 5H) 6.80 (br s, 1H) 3.45-3.84 (m, 5H) 2.67-3.16 (m, 7H) 2.33 (br d, J=11.03 Hz, 2H) 2.13 (br d, J=3.09 Hz, 1H) 1.70-1.92 (m, 3H) 1.58 (br s, 1H) 1.28 (br t, J=7.06 Hz, 4H)

LCMS (ESI+): m/z 416.2 (M+H)

### Example 9: General Protocol F for Synthesis of Exemplary Compounds

General Protocol F to synthesize exemplary compounds of Formula (I) is described in Scheme 6 and the procedures set forth below.

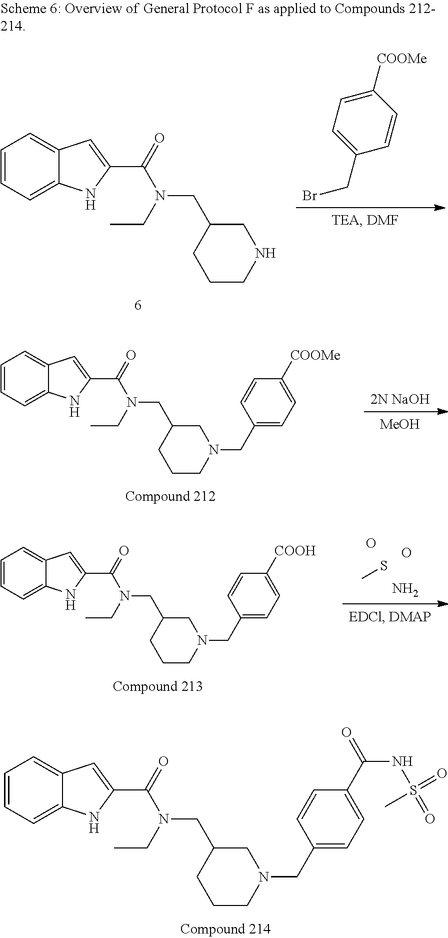

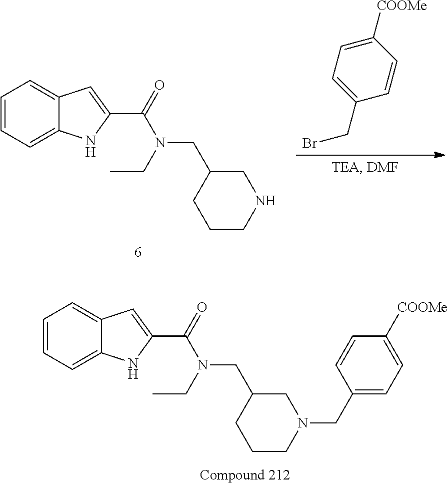

Procedure for the preparation of compound 212: A mixture of compound 6 (40.0 mg, 124.3 μmol, 1.0 eq, HCl salt), methyl 4-(bromomethyl) benzoate (31.3 mg, 136.7 μmol, 1.1 eq), TEA (62.9 mg, 621.4 μmol, 5.0 eq) in DMF (2 mL) was stirred at 25° C. for 1 hour. The reaction was monitored by LCMS and allowed to run until completion. The reaction mixture was extracted with two 4 mL portions of ethyl acetate. The combined organic layers were washed twice with 4 mL of sat. aqueous NH₄Cl, then dried over Na₂SO₄, filtered and concentrated under reduced pressure to give an oil. The residue was purified by prep-TLC (SiO₂, Petroleum ether/ethyl acetate=1:1) to give 13.9 mg of compound 212 (26% yield) as a white solid.

¹H NMR (400 MHz, CHLOROFORM-d) δ ppm 9.21-9.04 (m, 1H) 7.88 (d, J=7.8 Hz, 2H) 7.58 (d, J=7.8 Hz, 1H) 7.29 (br. s., 4H) 7.07 (d, J=7.4 Hz, 1H) 6.74 (br. s., 1H) 3.83 (s, 3H) 3.74-3.55 (m, 2H) 3.47 (br. s., 4H) 2.73-2.52 (m, 2H) 2.11-1.91 (m, 2H) 1.64 (br. s., 2H) 1.34-1.01 (m, 6H)

LCMS (ESI+): m/z 434.3 (M+H)

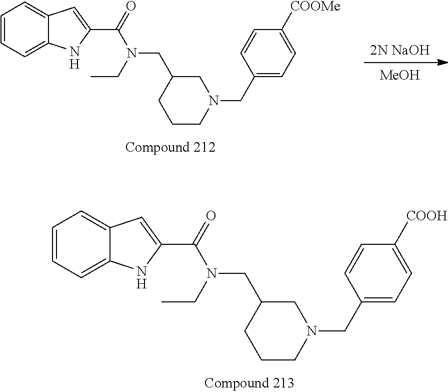 [SMILES: "MeOH" ; CCN(CC1CCCN(Cc2ccc(C(=O)OC)cc2)C1)C(=O)c1cc2ccccc2[nH]1 ; "2N NaOH" ; CCN(CC1CCCN(Cc2ccc(C(=O)O)cc2)C1)C(=O)c1cc2ccccc2[nH]1]

Procedure for the preparation of compound 213: A mixture of compound 212 (89.0 mg, 205.3 μmol, 1.0 eq) in NaOH (500 μL, 2M) and MeOH (2 mL) was stirred at 25° C. for 12 hours. The reaction was monitored by LCMS and allowed to run until completion. The reaction mixture was concentrated under reduced pressure to remove the methanol. The water was acidified to pH=5 with 10 percent aqueous HCl. The resulting solids were filtered, washed with water, and concentrated under reduced pressure to give 8.0 mg of compound 213 as a white solid. (8.5% yield, HCl salt)

¹H NMR (400 MHz, DMSO-d₆) δ ppm 12.84 (s, 2H) 11.54-11.46 (m, 1H) 7.89-7.81 (m, 2H) 7.60-7.54 (m, 1H) 7.43-7.29 (m, 3H) 7.18-7.11 (m, 1H) 7.04-6.97 (m, 1H) 6.70-6.63 (m, 1H) 3.59-3.42 (m, 4H) 3.30 (br. s., 2H) 2.70-2.63 (m, 1H) 1.98 (d, J=9.0 Hz, 2H) 1.87-1.75 (m, 1H) 1.62 (br. s., 2H) 1.46-1.37 (m, 1H) 1.14 (d, J=6.7 Hz, 3H) 1.04-0.92 (m, 1H)

LCMS (ESI+): m/z 380.1 (M+H)

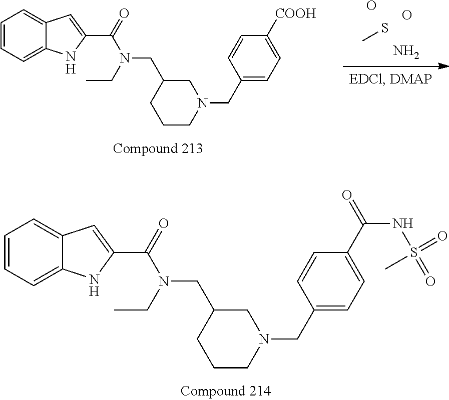

Procedure for the preparation of compound 214: A mixture of compound 213 (90.0 mg, 197.4 μmol, 1.0 eq, HCl salt), methanesulfonamide (20.7 mg, 217.1 μmol, 1.1 eq), EDCI (37.8 mg, 197.4 μmol, 1.0 eq), and DMAP (24.1 mg, 197.4 μmol, 1.0 eq) in 2 mL of DMF was stirred at 40° C. for 12 hours. The reaction was monitored by LCMS and allowed to run until completion. The reaction mixture was filtered. The residue was purified by prep-HPLC (TFA condition) to give 4.2 mg (4% as TFA salt) of compound 214 as a white solid

¹H NMR (400 MHz, METHANOL-d₄) δ ppm 7.95 (br. s., 2H) 7.62 (br. s., 3H) 7.46 (d, J=7.1 Hz, 1H) 7.24 (br. s., 1H) 7.08 (br. s., 1H) 6.80 (br. s., 1H) 4.38 (br. s., 2H) 3.83-3.63 (m, 3H) 3.45 (br. s., 3H) 3.33 (br. s., 2H) 3.00-2.78 (m, 2H) 2.33 (br. s., 1H) 2.05-1.90 (m, 2H) 1.77 (d, J=11.0 Hz, 1H) 1.30 (br. s., 5H)

LCMS (ESI+): m/z 497.1 (M+H)

### Example 10: General Protocol G for Synthesis of Exemplary Compounds

General Protocol G to synthesize exemplary compounds of Formula (I) is described in Scheme 7 and the procedures set forth below.

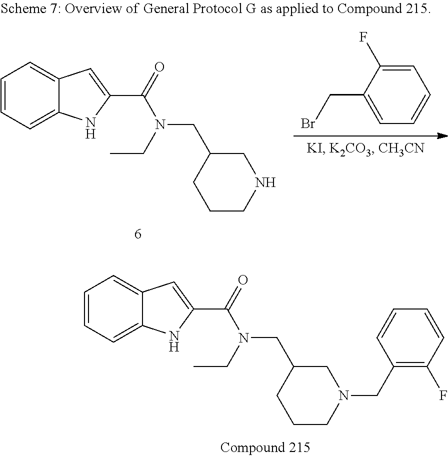

A mixture of compound 6 (50.0 mg, 125.2 μmol, 1.0 eq, TFA salt), KI (2.1 mg, 12.5 μmol, 0.1 eq), K₂CO₃ (51.9 mg, 375.5 μmol, 52.1 uL, 3.0 eq) in 2 mL of CH₃CN was added 1-(bromomethyl)-2-fluoro-benzene (23.7 mg, 125.2 μmol, 1.0 eq), then the mixture was stirred at 20° C. for 12 hours under N₂ atmosphere. The reaction was monitored by LCMS and allowed to run until complete. The mixture was filtered to give a yellow liquid which was purified by prep-HPLC (neutral condition) to give 11.4 mg of compound 215 (22.4% yield, 97% purity) as a light green solid.

¹H NMR (400 MHz, METHANOL-d₄) δ ppm 7.58 (d, J=8.38 Hz, 1H) 7.15-7.42 (m, 4H) 6.96-7.09 (m, 3H) 6.74 (s, 1H) 3.44-3.72 (m, 6H) 2.79 (br. s., 2H) 2.11 (br. s., 2H) 1.90 (br. s., 1H) 1.48-1.77 (m, 3H) 1.23 (t, J=6.84 Hz, 3H) 1.03 (br. s., 1H)

LCMS (ESI+): m/z 394.2 (M+H)

The following compounds were prepared analogously:

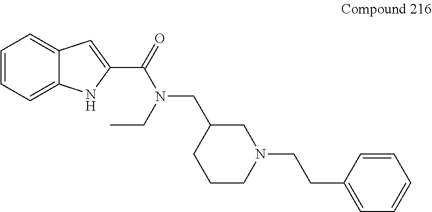 [SMILES: CCN(CC1CCCN(CCc2ccccc2)C1)C(=O)c1cc2ccccc2[nH]1]

¹H NMR (400 MHz, METHANOL-d₄) δ ppm 7.60 (d, J=7.94 Hz, 1H) 7.41 (d, J=8.38 Hz, 1H) 7.11-7.25 (m, 6H) 7.04 (t, J=7.28 Hz, 1H) 6.83 (s, 1H) 3.73 (br. s., 4H) 2.60-3.08 (m, 7H) 2.15 (d, J=11.47 Hz, 2H) 1.55-1.87 (m, 3H) 1.29 (t, J=6.84 Hz, 3H) 1.10 (br. s., 1H)

LCMS (ESI+): m/z 390.2 (M+H)

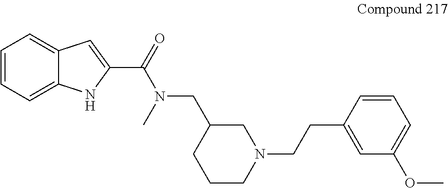 [SMILES: COc1cccc(CCN2CCCC(CN(C)C(=O)c3cc4ccccc4[nH]3)C2)c1]

¹H NMR (400 MHz, METHANOL-d₄) δ ppm 7.61 (d, J=8.38 Hz, 1H) 7.42 (d, J=7.94 Hz, 1H) 7.11-7.24 (m, 2H) 7.05 (t, J=7.50 Hz, 1H) 6.92 (br. s., 1H) 6.68-6.80 (m, 3H) 3.57-3.80 (m, 5H) 3.39 (br. s., 3H) 3.17 (br. s., 1H) 2.84 (br. s., 5H) 2.20 (br. s., 3H) 1.58-1.94 (m, 3H) 1.03-1.31 (m, 1H)

LCMS (ESI+): m/z 406.2 (M+H)

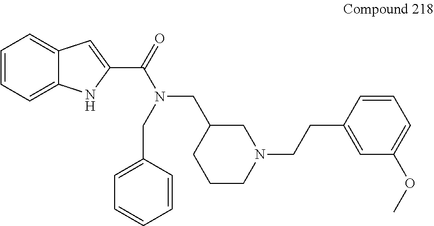 [SMILES: COc1cccc(CCN2CCCC(CN(Cc3ccccc3)C(=O)c3cc4ccccc4[nH]3)C2)c1]

¹H NMR (400 MHz, METHANOL-d₄) δ ppm 7.46-7.72 (m, 1H) 7.23-7.45 (m, 6H) 7.09-7.22 (m, 2H) 7.03 (d, J=7.50 Hz, 1H) 6.59-6.80 (m, 4H) 4.98 (br. s., 2H) 3.71 (s, 3H) 3.33-3.47 (m, 2H) 2.94 (br. s., 1H) 2.74 (br. s., 2H) 2.58 (br. s., 2H) 2.13 (br. s., 2H) 1.82-1.93 (m, 1H) 1.72 (br. s., 2H) 1.57 (br. s., 1H) 1.25-1.34 (m, 1H) 0.86-0.97 (m, 1H)

LCMS (ESI+): m/z 482.3 (M+H)

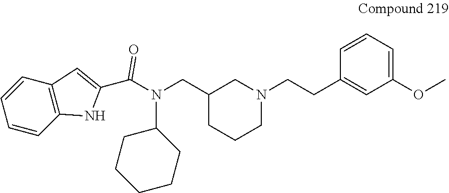 [SMILES: COc1cccc(CCN2CCCC(CN(C(=O)c3cc4ccccc4[nH]3)C3CCCCC3)C2)c1]

¹H NMR (400 MHz, CHLOROFORM-d) δ ppm 9.60 (br. s., 1H) 7.60 (d, J=8.38 Hz, 1H) 7.37 (d, J=8.38 Hz, 1H) 7.21-7.25 (m, 1H) 7.04-7.14 (m, 2H) 6.60-6.70 (m, 4H) 4.17 (t, J=11.69 Hz, 1H) 3.67-3.76 (m, 5H) 3.58 (d, J=11.03 Hz, 1H) 2.89-3.23 (m, 5H) 2.44-2.61 (m, 2H) 2.32 (br. s., 1H) 1.98 (d, J=9.70 Hz, 2H) 1.67-1.93 (m, 5H) 1.60 (d, J=12.79 Hz, 1H) 1.34-1.54 (m, 2H) 1.16-1.28 (m, 3H) 0.98-1.12 (m, 1H)

LCMS (ESI+): m/z 474.3 (M+H)

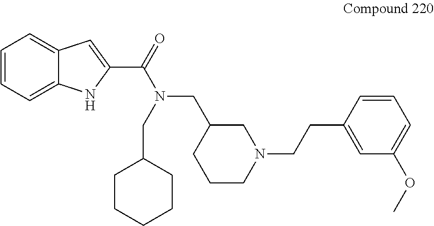 [SMILES: COc1cccc(CCN2CCCC(CN(CC3CCCCC3)C(=O)c3cc4ccccc4[nH]3)C2)c1]

¹H NMR (400 MHz, CHLOROFORM-d) δ ppm 9.97 (br. s., 1H) 7.62 (br. s., 1H) 7.39 (br. s., 1H) 7.10 (d, J=7.94 Hz, 2H) 6.58-6.81 (m, 5H) 3.97 (br. s., 1H) 3.68-3.81 (m, 5H) 3.59 (br. s., 2H) 2.92-3.30 (m, 7H) 2.32-2.59 (m, 4H) 1.78-2.09 (m, 4H) 0.92-1.32 (m, 6H) 0.65-0.86 (m, 2H)

LCMS (ESI+): m/z 380.2 (M+H)

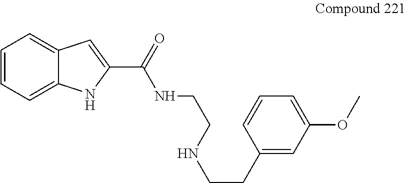 [SMILES: COc1cccc(CCNCCNC(=O)c2cc3ccccc3[nH]2)c1]

¹H NMR (400 MHz, CHLOROFORM-d) δ ppm 9.75 (br. s., 1H) 9.19 (br. s., 1H) 7.99 (br. s., 1H) 7.50 (d, J=7.94 Hz, 1H) 7.30 (d, J=8.38 Hz, 1H) 7.20 (t, J=7.50 Hz, 1H) 7.01-7.14 (m, 2H) 6.94 (s, 1H) 6.61-6.71 (m, 3H) 3.61-3.75 (m, 5H) 3.17 (br. s., 4H) 2.93 (br. s., 2H)

LCMS (ESI+): m/z 338.1 (M+H)

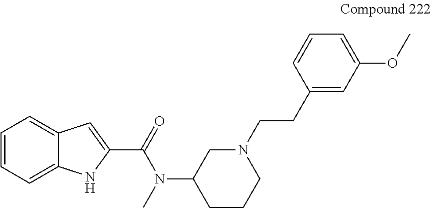 [SMILES: COc1cccc(CCN2CCCC(N(C)C(=O)c3cc4ccccc4[nH]3)C2)c1]

¹H NMR (400 MHz, CHLOROFORM-d) δ ppm 7.62 (d, J=7.9 Hz, 1H), 7.44 (d, J=7.9 Hz, 1H), 7.25-7.13 (m, 2H), 7.07 (t, J=7.3 Hz, 1H), 6.89-6.71 (m, 4H), 3.75 (s, 3H), 3.32-3.31 (m, 3H), 3.13 (d, J=9.3 Hz, 3H), 2.98 (d, J=11.9 Hz, 1H), 2.86-2.76 (m, 2H), 2.72-2.60 (m, 2H), 2.33 (br. s., 1H), 2.05-1.74 (m, 4H)

LCMS (ESI+): m/z 392.2 (M+H)

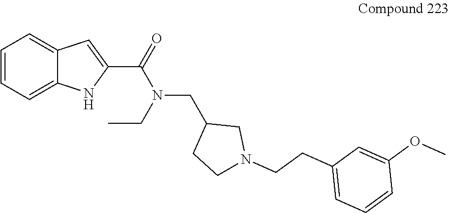 [SMILES: CCN(CC1CCN(CCc2cccc(OC)c2)C1)C(=O)c1cc2ccccc2[nH]1]

Used CH₃CN/K₂CO₃ as a solvent/base system analogously.

¹H NMR (400 MHz, METHANOL-d₄) δ ppm 7.63 (d, J=7.94 Hz, 1H), 7.44 (d, J=8.38 Hz, 1H), 7.27-7.18 (m, 2H), 7.07 (t, J=7.50 Hz, 1H), 6.92-6.76 (m, 4H), 3.87-3.66 (m, 7H), 3.44 (d, J=7.06 Hz, 2H), 3.18 (dd, J=18.08, 10.14 Hz, 1H), 3.09-2.77 (m, 4H), 2.42-2.11 (m, 2H), 2.06-1.72 (m, 2H), 1.34 (t, J=6.84 Hz, 3H)

LCMS (ESI+): m/z 406.2 (M+H)

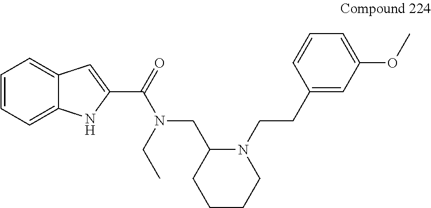 [SMILES: CCN(CC1CCCCN1CCc1cccc(OC)c1)C(=O)c1cc2ccccc2[nH]1]

Used CH₃CN/K₂CO₃ as a solvent/base system analogously

¹H NMR (400 MHz, METHANOL-d4) δ ppm 7.64 (d, J=7.9 Hz, 1H), 7.44 (d, J=8.4 Hz, 1H), 7.28-7.14 (m, 2H), 7.08 (t, J=7.5 Hz, 1H), 6.97-6.85 (m, 3H), 6.84-6.66 (m, 1H), 4.35-4.19 (m, 1H), 3.92-3.73 (m, 4H), 3.65 (s, 3H), 3.59-3.32 (m, 3H), 3.24-2.99 (m, 3H), 2.17-1.67 (m, 6H), 1.37 (t, J=7.1 Hz, 3H)

LCMS (ESI+): m/z 420.2 (M+H)

### Example 11: General Protocol H for Synthesis of Exemplary Compounds

General Protocol H to synthesize exemplary compounds of Formula (I) is described in Scheme 8 and the procedures set forth below.

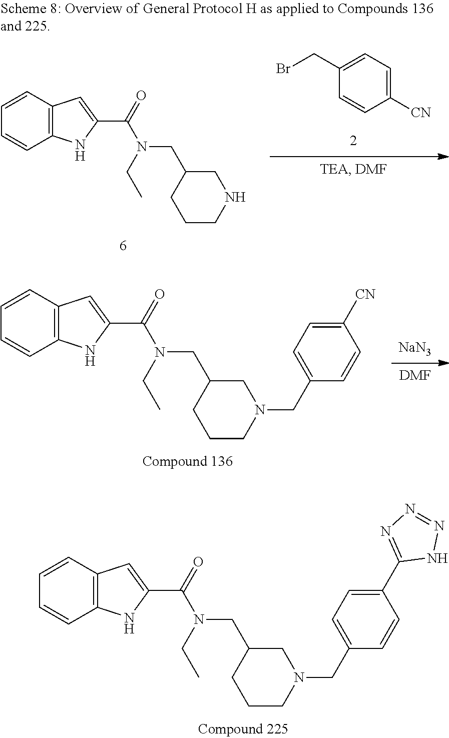

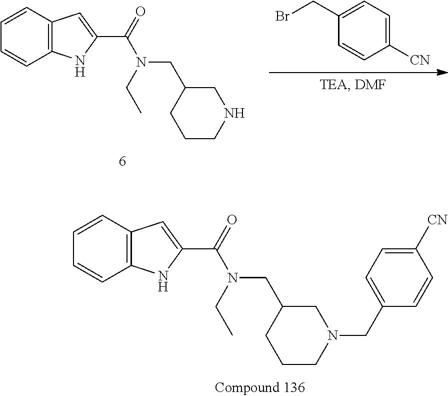

Procedure for the preparation of compound 136: To a mixture of compound 6 (80.0 mg, 248.6 μmol, 1.0 eq, HCl salt) and TEA (125.8 mg, 1.2 mmol, 5.0 eq) in 2 mL of DMF was added 4-cyanobenzyl bromide (58.5 mg, 298.3 μmol, 1.2 eq) at 15° C. and the reaction was stirred for 1 h at 15° C. The reaction was monitored by MS and allowed to run until complete. The reaction mixture was diluted with 5 mL of water, extracted with three 5 mL portions of ethyl acetate. The combined organic layers were washed twice with 10 mL of brine, dried over Na₂SO₄, filtered and the filtrate was concentrated in vacuo. The residue was purified by prep-TLC (SiO₂ eluting with ethyl acetate) to give 73 mg of compound 136 (73% yield) as a colorless gum.

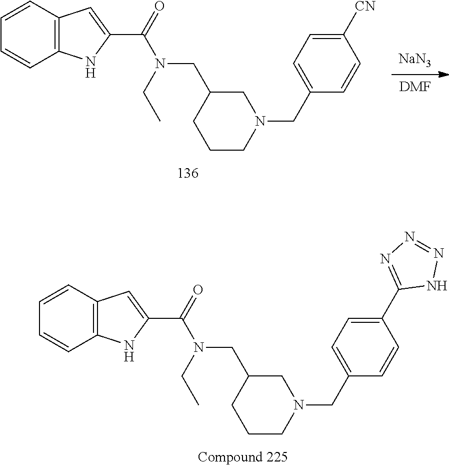

Procedure for the preparation of compound 225: To a solution of compound 136 (35.0 mg, 87.4 μmol, 1.0 eq) in 2 mL of DMF was added NaN₃ (6.3 mg, 96.1 μmol, 1.1 eq) and NH₄Cl (5.1 mg, 96.1 μmol, 1.1 eq) at 15° C. and the reaction was stirred for 12 hrs at 110° C. The reaction was monitored by LCMS and allowed to run until completion. The reaction mixture was filtered and the filtrate was purified by prep-HPLC (TFA condition) to give 4.7 mg of compound 225 (9.7% yield, TFA salt) as a light yellow solid.

¹H NMR (400 MHz, METHANOL-d₄) δ ppm 8.09 (br s, 1H), 7.93 (br s, 1H), 7.69 (br s, 2H), 7.37-7.53 (m, 2H), 7.20 (br s, 1H), 7.02 (br s, 1H), 6.74 (br s, 1H), 4.39 (br s, 2H), 3.61-4.27 (m, 3H), 3.37-3.53 (m, 2H), 2.79-3.26 (m, 3H), 2.24-2.65 (m, 2H), 1.87-2.18 (m, 2H), 1.77 (br s, 1H), 1.29 (br s, 3H).

LCMS (ESI+): m/z 444.2 (M+H)

### Example 12: General Protocol I for Synthesis of Exemplary Compounds

General Protocol I to synthesize exemplary compounds of Formula (I) is described in Scheme 9 and the procedures set forth below.

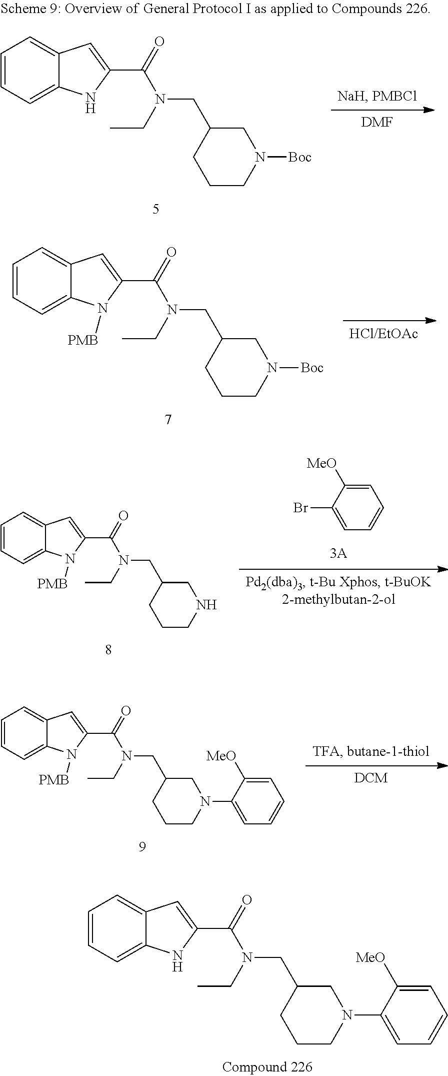

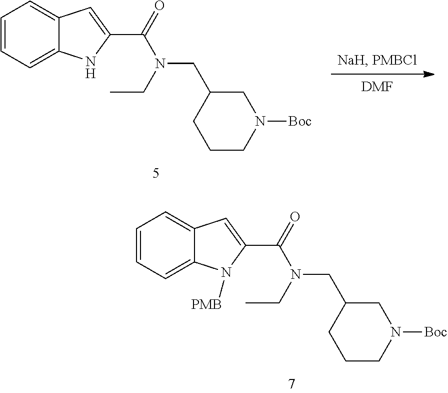

Procedure for the preparation of compound 7: To the mixture of compound 5 (200.0 mg, 518.8 μmol, 1.0 eq) in 3 mL of DMF was added NaH (24.9 mg, 622.6 μmol, 60% purity, 1.2 eq) at 0° C. The mixture was stirred at 0° C. for 30 mins. Then p-methoxybenzyl chloride (89.4 mg, 570.7 μmol, 1.1 eq) was added and the reaction mixture was stirred at 15° C. for 3 hours. The reaction was monitored by TLC and allowed to run until complete. The mixture was poured into 20 mL of water to quench the reaction and extracted with three 5 mL portions of ethyl acetate.

The combined organic phase was washed twice with 10 mL of brine, dried with anhydrous Na₂SO₄, filtered and concentrated in vacuum to give 300 mg of compound 7 as a light yellow oil.

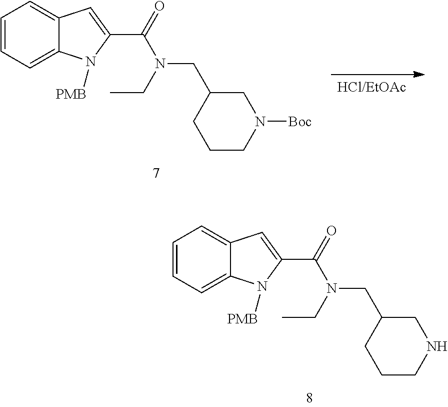

Procedure for the preparation of compound 8: The mixture of compound 7 (490.0 mg, 969.1 μmol, 1.0 eq) in HCl/ethyl acetate (10 mL) was stirred at 15° C. for 1 hour. The reaction was monitored by TLC and allowed to run until complete. The reaction was concentrated in vacuum. The residue was dissolved in 10 mL of H₂O, and adjusted by saturated Na₂CO₃ to pH=7, and extracted with four 5 mL portions of ethyl acetate. The combined organic phase was dried with anhydrous Na₂SO4, filtered and concentrated in vacuum to give 390 mg of compound 8 as a light yellow oil.

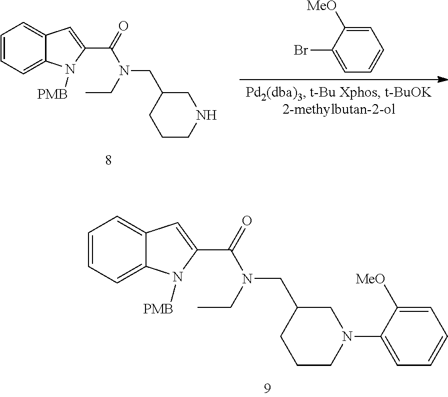

Procedure for the preparation of compound 9: The mixture of compound 8 (130.0 mg, 320.6 μmol, 1.0 eq), 2-bromoanisole (90.0 mg, 384.7 μmol, 1.2 eq), t-BuOK (71.9 mg, 641.1 μmol, 2.0 eq), t-Bu Xphos (13.6 mg, 32.1 μmol, 0.1 eq) and Pd₂(dba)₃ (29.4 mg, 32.1 μmol, 0.1 eq) in 2-methylbutan-2-ol (2 mL) was stirred at 120° C. for 24 hours. The reaction was monitored by TLC and allowed to run until complete. The mixture was concentrated in vacuum. The residue was poured into 20 mL of water and extracted with three 10 mL portions of ethyl acetate. The combined organic phase was dried with anhydrous Na₂SO₄, filtered and concentrated in vacuum. The residue was purified by prep-TLC (SiO₂ eluting with petroleum ether/ethyl acetate=1/1) to give 40 mg (24%) of compound 9 as a light yellow solid.

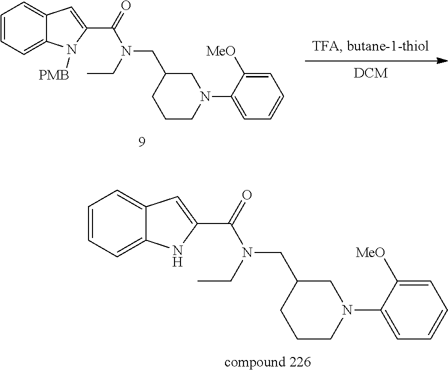

Procedure for the preparation of compound 226: To a solution of compound 9 (40.0 mg, 78.2 μmol, 1.0 eq) in DCM (500 uL) was added butane-1-thiol (168.0 mg, 1.9 mmol, 23.8 eq) and TFA (770.0 mg, 6.8 mmol, 86.4 eq). The mixture was stirred at 15° C. for 16 hours. The reaction was monitored by LCMS. The reaction was concentrated in vacuum. The residue was purified by prep-HPLC (TFA condition) to give 14.1 mg (33% yield, as TFA salt) of compound 226 as a colorless gum.

¹H NMR (400 MHz, METHANOL-d₄) δ (400 MHz, METHANOL-d) δ ppm 7.62 (d, J=7.50 Hz, 2H) 7.52 (t, J=7.50 Hz, 1H) 7.42 (d, J=7.94 Hz, 1H) 7.11-7.31 (m, 3H) 7.07 (t, J=6.84 Hz, 1H) 6.88 (br. s., 1H) 3.98 (br. s., 3H) 3.81 (br. s., 3H) 3.46-3.69 (m, 4H) 2.65 (br. s., 1H) 1.97-2.19 (m, 3H) 1.66 (d, J=6.17 Hz, 2H) 1.44-1.57 (m, 1H) 1.36 (br. s., 3H)

LCMS (ESI+): m/z 392.2 (M+H)

The following compounds were prepared analogously according to Method “I”:

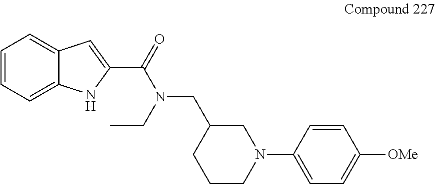 [SMILES: CCN(CC1CCCN(c2ccc(OC)cc2)C1)C(=O)c1cc2ccccc2[nH]1]

¹H NMR (400 MHz, METHANOL-d₄) δ (400 MHz, METHANOL-d) δ ppm 7.53-7.65 (m, 2H) 7.42 (d, J=7.94 Hz, 1H) 7.22 (t, J=7.06 Hz, 1H) 7.01-7.13 (m, 3H) 6.89 (br. s., 1H) 3.70-3.89 (m, 5H) 3.38-3.67 (m, 5H) 2.57 (br. s., 1H) 2.16 (br. s., 1H) 2.03 (br. s., 2H) 1.66 (d, J=6.62 Hz, 2H) 1.55 (br. s., 1H) 1.37 (br. s., 3H)

LCMS (ESI+): m/z 392.2 (M+H)

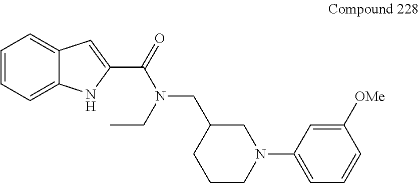 [SMILES: CCN(CC1CCCN(c2cccc(OC)c2)C1)C(=O)c1cc2ccccc2[nH]1]

¹H NMR (400 MHz, METHANOL-d₄) δ (400 MHz, METHANOL-d) δ ppm 7.62 (d, J=7.94 Hz, 1H) 7.38-7.49 (m, 2H) 7.10-7.26 (m, 2H) 6.99-7.10 (m, 2H) 6.89 (br. s., 1H) 3.72-3.93 (m, 5H) 3.64 (br. s., 2H) 3.48 (br. s., 3H) 2.54 (br. s., 1H) 2.12 (br. s., 1H) 1.90-2.06 (m, 2H) 1.66 (d, J=6.62 Hz, 2H) 1.46-1.60 (m, 1H) 1.36 (br. s., 3H)

LCMS (ESI+): m/z 392.3 (M+H)

### Example 13: General Protocol J for Synthesis of Exemplary Compounds

General Protocol J to synthesize exemplary compounds of Formula (I) is described in Scheme 10 and the procedures set forth below.

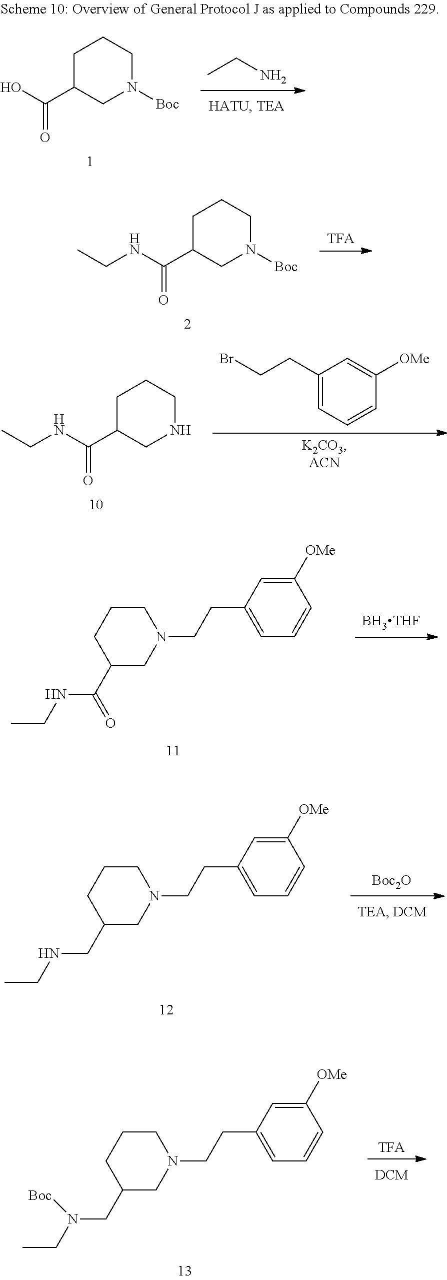

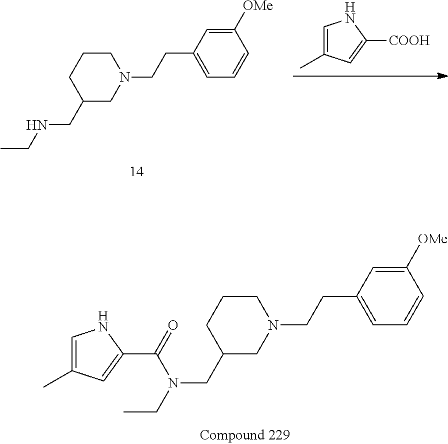

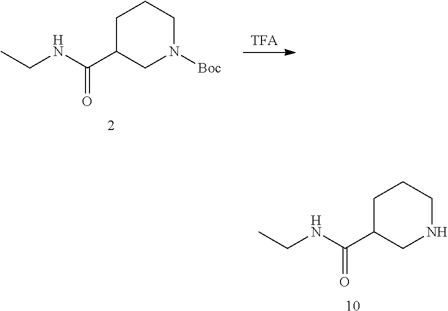

Procedure for the preparation of compound 10: To a solution of compound 2 (8.0 g, 31.2 mmol, 1.0 eq) in TFA (15 mL) and 75 mL of CH₂Cl₂. The mixture was stirred at 25° C. for 1 hour. The reaction was monitored by LC-MS and allowed to run until complete. The reaction mixture was concentrated under reduced pressure to give 10.0 g of compound 10 as an oil. The material was used in subsequent steps without further purification.

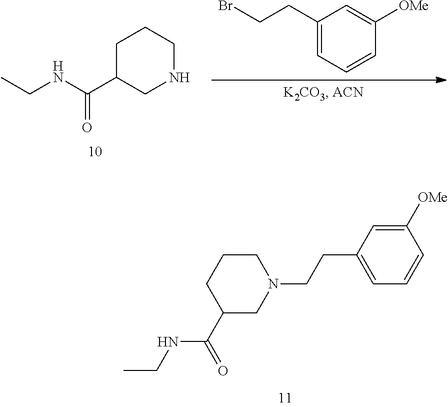

Procedure for the preparation of compound 11: A mixture of compound 10 (7.7 g, 28.5 mmol, 1.0 eq, TFA) and K₂CO₃ (19.7 g, 142.5 mmol, 5.0 eq) and KI (473.0 mg, 2.9 mmol, 0.1 eq) in 80n mL of ACN was stirred at 25° C., then 1-(2-bromoethyl)-3-methoxybenzene (6.1 g, 28.5 mmol, 1.0 eq) was added at 25° C. for 0.5 hour, and then the mixture was stirred at 45° C. for 11.5 hours. The reaction was monitored by LC-MS and allowed to run until complete. The reaction mixture was concentrated under reduced pressure to give a residue, then diluted with water and adjusted to pH˜3 with 6N HCl. It was washed twice with 60 mL of TBME. Then the water layers were made basic with NaOH to pH˜10). The mixture was extracted with five 50 mL portions of ethyl acetate. The combined organic layers were washed twice with 50 mL of brine, dried over Na₂SO₄, filtered and concentrated under reduced pressure to give 4.3 g of compound 11 as a brown oil. This material was used in the next step without further purification.

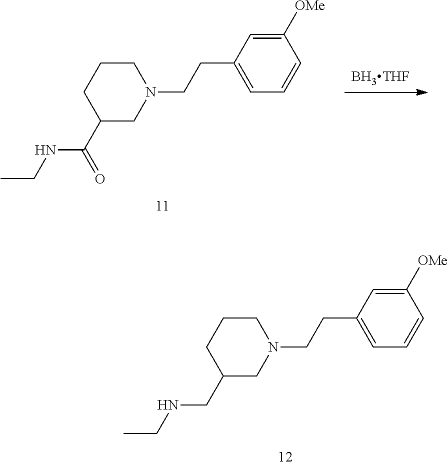 [SMILES: CCNC(=O)C1CCCN(CCc2cccc(OC)c2)C1 ; "BH3·THF" ; CCNCC1CCCN(CCc2cccc(OC)c2)C1]

Procedure for the preparation of compound 12: To a solution of compound 11 (4.3 g, 15.9 mmol, 1.0 eq) in 50 mL of THF (50 mL) was added BH₃.THF (1 M, 47.8 mL, 3.0 eq) at 0° C. The mixture was stirred at 70° C. for 4 hours. The reaction was monitored by LC-MS and allowed to run until complete. The mixture was cooled in an ice bath, quenched by adding 25 mL of 10% aqueous HCl and 8 mL of MeOH, then the mixture was stirred at 65° C. for 2 hours. To the mixture was added HCl/MeOH (30 mL) and it was stirred at 65° C. for 1.5 hours. It was concentrated to afford 5.3 g of the HCl salt of compound 12 as a yellow oil.

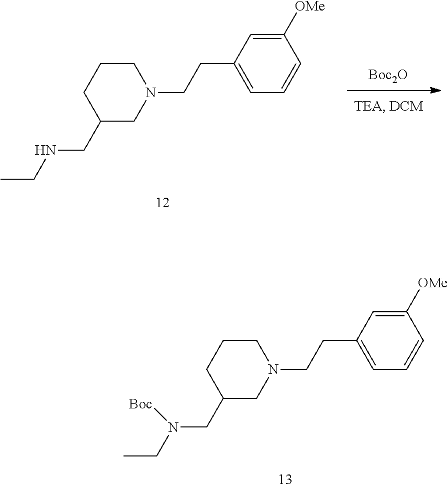 [SMILES: "Boc2O" ; CCNCC1CCCN(CCc2cccc(OC)c2)C1 ; CCN(CC1CCCN(CCc2cccc(OC)c2)C1)C(=O)OC(C)(C)C]

Procedure for the preparation of compound 13: A mixture of compound 12 (2.5 g, 8.0 mmol, 1.0 eq, HCl), Boc₂O (3.5 g, 16.0 mmol, 3.7 mL, 2.0 eq), TEA (4.0 g, 40.0 mmol, 5.0 eq) in DCM was stirred at 25° C. for 12 hours. The reaction was monitored by LC-MS and allowed to run until complete. The reaction mixture was washed five times with 20 mL of saturated NH₄Cl solution, dried over Na₂SO₄, filtered and concentrated under reduced pressure to give an oil. The oil was purified by column chromatography (SiO2 eluting with petroleum ether/ethyl acetate=30/1 to 0/1) to give 3.0 g of compound 13 (˜99%) as a yellow oil. The material was used without further purification directly in the next reaction.

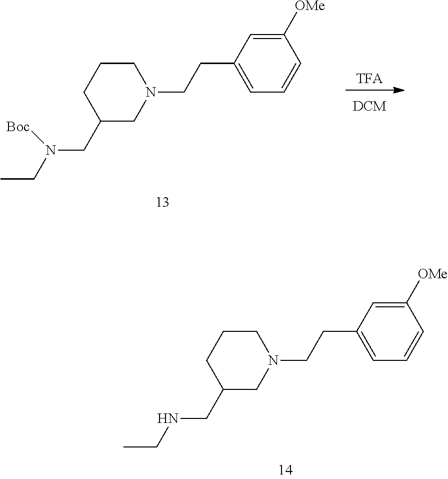 [SMILES: "TFA" ; CCN(CC1CCCN(CCc2cccc(OC)c2)C1)C(=O)OC(C)(C)C ; CCNCC1CCCN(CCc2cccc(OC)c2)C1]

Procedure for the preparation of compound 14: A mixture of compound 13 (1.0 g, 2.7 mmol, 1.0 eq) in 2 mL of TFA and 10 mL of DCM was stirred at 25° C. for 12 hours. The reaction was monitored by LC-MS and allowed to run until completion. The reaction mixture was concentrated under reduced pressure to give 1.9 g of compound 14 (TFA salt) as a yellow oil.

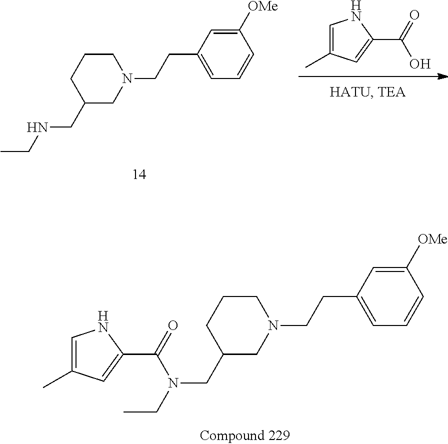

Procedure for the preparation of compound 229: A mixture of 4-methyl-1H-pyrrole-2-carboxylic acid (33.7 mg, 268.9 μmol, 1.5 eq), HATU (81.8 mg, 215.1 μmol, 1.2 eq), TEA (90.7 mg, 896.4 μmol, 5.0 eq) in 2 mL of DMF was stirred at 25° C. for 0.5 hour, then compound 14 (70.0 mg, 179.3 μmol, 1.0 eq, TFA) was added at 25° C., and then the mixture was stirred at 40° C. for 11.5 hours. The reaction was monitored by LC-MS and allowed to run until complete. The reaction mixture was filtered. The residue was purified by prep-HPLC (TFA condition) to give 16.1 mg of the TFA salt of compound 229 as a green oil (18% yield).

¹H NMR (400 MHz, METHANOL-d₄) δ ppm 7.22 (t, J=7.94 Hz, 1H) 6.76-6.88 (m, 3H) 6.73 (s, 1H) 6.46 (s, 1H) 3.77 (s, 3H) 3.46-3.72 (m, 4H) 3.33-3.40 (m, 2H) 2.88-3.23 (m, 4H) 2.74-2.83 (m, 1H) 2.26-2.46 (m, 1H) 2.08-2.16 (m, 3H) 1.98-2.07 (m, 1H) 1.90 (d, J=11.47 Hz, 3H) 1.23-1.40 (m, 4H)

LCMS (ESI+): m/z 384.2 (M+H)

The following compounds were prepared analogously:

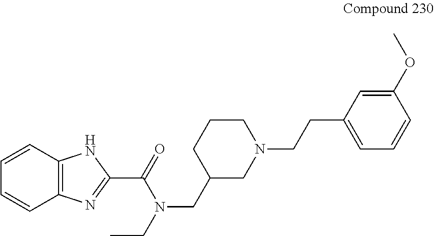 [SMILES: CCN(CC1CCCN(CCc2cccc(OC)c2)C1)C(=O)c1nc2ccccc2[nH]1]

¹H NMR (400 MHz, METHANOL-d₄) δ ppm 7.63-7.77 (m, 2H) 7.30-7.46 (m, 2H) 6.94-7.25 (m, 1H) 6.75-6.87 (m, 2H) 6.45-6.59 (m, 1H) 3.94-4.07 (m, 1H) 3.47-3.83 (m, 7H) 3.32-3.45 (m, 3H) 2.81-3.24 (m, 4H) 2.39 (br. s., 1H) 1.97 (br. s., 3H) 1.19-1.48 (m, 4H)

LCMS (ESI+): m/z 421.2 (M+H)

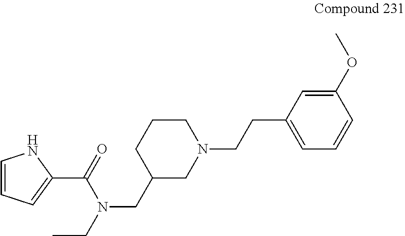 [SMILES: CCN(CC1CCCN(CCc2cccc(OC)c2)C1)C(=O)c1ccc[nH]1]

¹H NMR (400 MHz, METHANOL-d₄) δ ppm 7.18-7.25 (m, 1H) 6.93-7.02 (m, 1H) 6.84 (br. s., 3H) 6.62-6.76 (m, 1H) 6.21-6.29 (m, 1H) 3.77 (s, 3H) 3.47-3.74 (m, 5H) 3.33-3.44 (m, 3H) 3.01 (d, J=8.11 Hz, 4H) 2.24-2.49 (m, 1H) 1.99-2.15 (m, 1H) 1.69-1.95 (m, 2H) 1.24-1.39 (m, 4H)

LCMS (ESI+): m/z 370.2 (M+H)

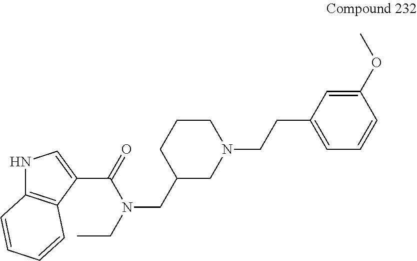 [SMILES: CCN(CC1CCCN(CCc2cccc(OC)c2)C1)C(=O)c1c[nH]c2ccccc12]

¹H NMR (400 MHz, METHANOL-d₄) δ ppm 7.65-7.73 (m, 1H) 7.61 (s, 1H) 7.41-7.49 (m, 1H) 7.08-7.26 (m, 3H) 6.83 (br. s., 3H) 3.71-3.80 (m, 3H) 3.46-3.69 (m, 5H) 3.32-3.38 (m, 2H) 2.67-3.23 (m, 5H) 2.30-2.52 (m, 1H) 1.72-2.07 (m, 3H) 1.16-1.37 (m, 4H)

LCMS (ESI+): m/z 420.2 (M+H)

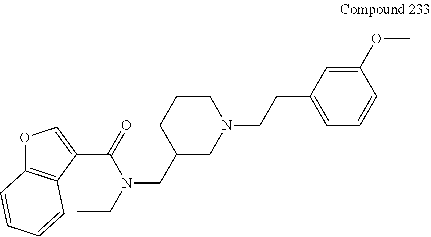 [SMILES: CCN(CC1CCCN(CCc2cccc(OC)c2)C1)C(=O)c1coc2ccccc12]

¹H NMR (400 MHz, METHANOL-d₄) δ ppm 8.10-8.19 (m, 1H) 7.66-7.71 (m, 1H) 7.56-7.60 (m, 1H) 7.40 (s, 1H) 7.34 (s, 1H) 7.24 (t, J=7.94 Hz, 1H) 6.79-6.87 (m, 3H) 3.72-3.81 (m, 3H) 3.54-3.68 (m, 4H) 3.46-3.53 (m, 1H) 3.37 (br. s., 2H) 3.03 (br. s., 5H) 2.30-2.52 (m, 1H) 1.76-2.09 (m, 3H) 1.14-1.46 (m, 4H)

LCMS (ESI+): m/z 421.2 (M+H)

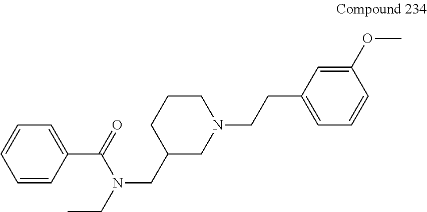 [SMILES: CCN(CC1CCCN(CCc2cccc(OC)c2)C1)C(=O)c1ccccc1]

¹H NMR (400 MHz, METHANOL-d₄) δ ppm 7.32-7.51 (m, 5H) 7.21-7.28 (m, 1H) 6.79-6.90 (m, 3H) 3.73-3.82 (m, 3H) 3.50-3.70 (m, 3H) 3.33-3.46 (m, 4H) 2.79-3.11 (m, 4H) 2.32-2.51 (m, 1H) 1.68-2.12 (m, 4H) 1.03-1.43 (m, 4H)

LCMS (ESI+): m/z 381.2 (M+H)

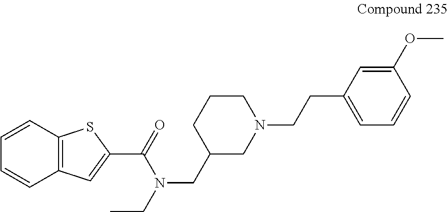 [SMILES: CCN(CC1CCCN(CCc2cccc(OC)c2)C1)C(=O)c1cc2ccccc2s1]

¹H NMR (400 MHz, METHANOL-d₄) δ ppm 7.87-7.95 (m, 2H) 7.66-7.78 (m, 1H) 7.40-7.47 (m, 2H) 7.20-7.29 (m, 1H) 6.77-6.92 (m, 3H) 3.77 (s, 3H) 3.43-3.71 (m, 5H) 3.36 (d, J=8.38 Hz, 2H) 2.75-3.19 (m, 4H) 2.32-2.53 (m, 1H) 1.69-2.14 (m, 4H) 1.29 (s, 4H)

LCMS (ESI+): m/z 437.1 (M+H)

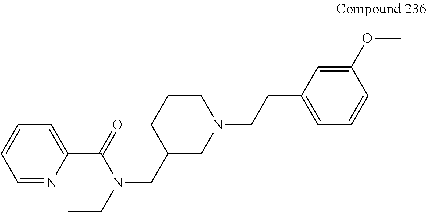 [SMILES: CCN(CC1CCCN(CCc2cccc(OC)c2)C1)C(=O)c1ccccn1]

¹H NMR (400 MHz, METHANOL-d₄) δ ppm 8.59-8.65 (m, 1H) 7.91-8.02 (m, 1H) 7.47-7.66 (m, 2H) 7.25 (s, 1H) 6.86 (br. s., 3H) 3.73-3.82 (m, 3H) 3.50-3.72 (m, 3H) 3.34-3.46 (m, 4H) 2.82-3.11 (m, 4H) 2.34-2.52 (m, 1H) 1.70-2.11 (m, 4H) 1.29 (br. s., 2H) 1.11 (t, J=7.06 Hz, 2H)

LCMS (ESI+): m/z 382.2 (M+H)

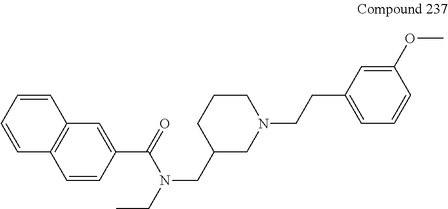 [SMILES: CCN(CC1CCCN(CCc2cccc(OC)c2)C1)C(=O)c1ccc2ccccc2c1]

¹H NMR (400 MHz, METHANOL-d₄) δ ppm 7.83-8.03 (m, 4H) 7.41-7.61 (m, 3H) 7.20-7.28 (m, 1H) 6.76-6.92 (m, 3H) 3.77 (s, 3H) 3.35-3.71 (m, 6H) 3.07 (br. s., 4H) 2.33-2.52 (m, 1H) 2.00 (d, J=13.23 Hz, 4H) 1.24-1.49 (m, 2H) 1.13 (br. s., 2H) 0.81-1.03 (m, 1H)

LCMS (ESI+): m/z 431.2 (M+H)

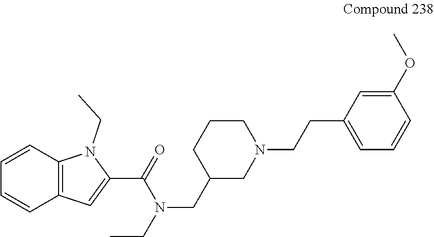 [SMILES: CCN(CC1CCCN(CCc2cccc(OC)c2)C1)C(=O)c1cc2ccccc2n1CC]

¹H NMR (400 MHz, METHANOL-d₄) δ ppm 7.61 (d, J=7.83 Hz, 1H) 7.46 (d, J=8.22 Hz, 1H) 7.19-7.31 (m, 2H) 7.10 (t, J=7.43 Hz, 1H) 6.78-6.90 (m, 3H) 6.64-6.77 (m, 1H) 4.24-4.34 (m, 2H) 3.72-3.80 (m, 3H) 3.46-3.68 (m, 5H) 3.36 (d, J=7.43 Hz, 2H) 3.17-3.25 (m, 1H) 3.04 (d, J=5.48 Hz, 3H) 2.32-2.53 (m, 1H) 2.05 (br. s., 4H) 1.37 (t, J=7.24 Hz, 3H) 1.31 (t, J=7.24 Hz, 1H) 1.23 (br. s., 3H)

LCMS (ESI+): m/z 448.2 (M+H)

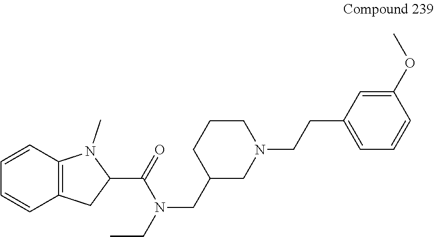 [SMILES: CCN(CC1CCCN(CCc2cccc(OC)c2)C1)C(=O)C1Cc2ccccc2N1C]

¹H NMR (400 MHz, METHANOL-d₄) δ ppm 7.21-7.14 (m, 1H) 7.07-6.96 (m, 2H) 6.87-6.70 (m, 3H) 6.67-6.58 (m, 1H) 6.53-6.45 (m, 1H) 4.40 (s, 1H) 3.76 (d, J=7.8 Hz, 3H) 3.57-3.35 (m, 4H) 3.03-2.77 (m, 5H) 2.73-2.60 (m, 4H) 1.93 (br. s., 4H) 1.82-1.59 (m, 3H) 1.25 (t, J=7.0 Hz, 3H) 1.18-1.05 (m, 2H)

LCMS (ESI+): m/z 436.2 (M+H)

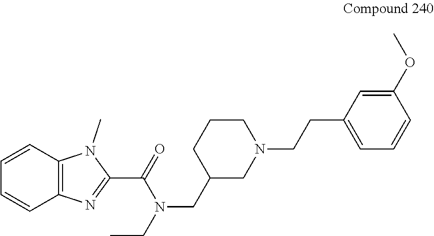 [SMILES: CCN(CC1CCCN(CCc2cccc(OC)c2)C1)C(=O)c1nc2ccccc2n1C]

¹H NMR (400 MHz, METHANOL-d₄) δ ppm 7.74-7.67 (m, 1H) 7.63-7.57 (m, 1H) 7.44-7.31 (m, 2H) 7.20-7.12 (m, 1H) 6.82-6.69 (m, 3H) 3.87 (s, 3H) 3.75 (s, 3H) 3.69-3.62 (m, 1H) 3.58-3.46 (m, 3H) 3.13-2.96 (m, 2H) 2.87-2.76 (m, 2H) 2.67 (d, J=7.4 Hz, 2H) 2.56-2.47 (m, 1H) 2.24-2.11 (m, 1H) 2.04-1.96 (m, 1H) 1.93-1.78 (m, 2H) 1.68-1.49 (m, 2H) 1.32 (t, J=7.2 Hz, 2H) 1.15 (t, J=7.0 Hz, 3H)

LCMS (ESI+): m/z 435.2 (M+H)

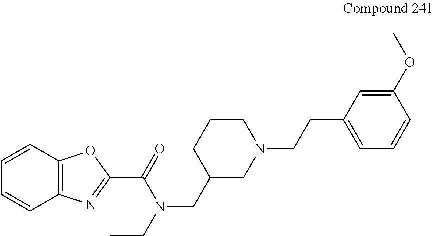 [SMILES: CCN(CC1CCCN(CCc2cccc(OC)c2)C1)C(=O)c1nc2ccccc2o1]

¹H NMR (400 MHz, METHANOL-d₄) δ ppm 7.84 (d, J=7.9 Hz, 1H) 7.71 (s, 1H) 7.58-7.45 (m, 2H) 7.24-7.16 (m, 1H) 6.83 (br. s., 3H) 3.91-3.81 (m, 1H) 3.75-3.71 (m, 3H) 3.67-3.56 (m, 3H) 3.50-3.44 (m, 1H) 3.36 (d, J=8.4 Hz, 3H) 3.06-2.82 (m, 4H) 2.52-2.31 (m, 1H) 2.09-1.89 (m, 2H) 1.85-1.71 (m, 1H) 1.40-1.25 (m, 4H)

LCMS (ESI+): m/z 422.2 (M+H)

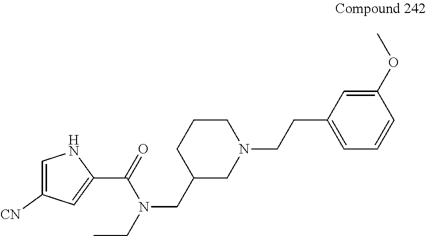

¹H NMR (400 MHz, METHANOL-d₄) δ ppm 7.55 (s, 1H) 7.22 (s, 1H) 7.03-6.92 (m, 1H) 6.85-6.77 (m, 3H) 3.76 (s, 3H) 3.66-3.47 (m, 5H) 3.33 (d, J=8.8 Hz, 3H) 3.00 (d, J=8.8 Hz, 3H) 2.79 (s, 1H) 2.45-2.21 (m, 1H) 2.07-1.99 (m, 1H) 1.93-1.68 (m, 2H) 1.28 (s, 4H)

LCMS (ESI+): m/z 395.2 (M+H)

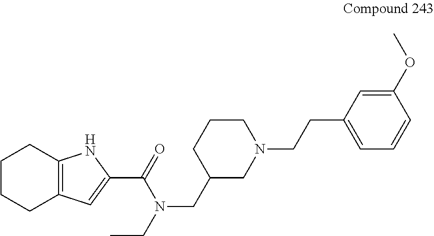 [SMILES: CCN(CC1CCCN(CCc2cccc(OC)c2)C1)C(=O)c1cc2c([nH]1)CCCC2]

¹H NMR (400 MHz, METHANOL-d₄) δ ppm 7.22 (t, J=8.0 Hz, 1H) 6.89-6.76 (m, 3H) 6.36 (s, 1H) 3.80-3.73 (m, 3H) 3.71-3.45 (m, 4H) 3.39-3.33 (m, 2H) 3.01 (d, J=5.9 Hz, 5H) 2.83-2.73 (m, 1H) 2.59 (t, J=5.9 Hz, 2H) 2.51 (d, J=5.5 Hz, 2H) 2.36-2.22 (m, 1H) 2.13-1.98 (m, 1H) 1.94-1.68 (m, 6H) 1.37-1.23 (m, 4H)

LCMS (ESI+): m/z 424.2 (M+H)

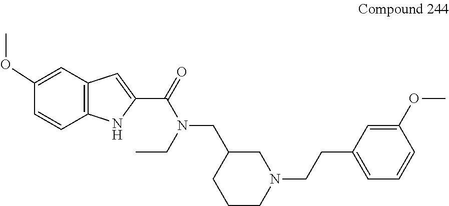 [SMILES: CCN(CC1CCCN(CCc2cccc(OC)c2)C1)C(=O)c1cc2cc(OC)ccc2[nH]1]

¹H NMR (400 MHz, METHANOL-d₄) δ ppm 7.31-7.37 (m, 1H) 7.21 (t, J=8.11 Hz, 1H) 7.09-7.13 (m, 1H) 6.77-6.93 (m, 5H) 3.81 (s, 4H) 3.70-3.78 (m, 4H) 3.59-3.70 (m, 2H) 3.54 (br d, J=10.52 Hz, 1H) 3.32-3.38 (m, 2H) 3.01 (br d, J=7.45 Hz, 2H) 2.86-2.98 (m, 1H) 2.35 (br s, 1H) 1.97-2.12 (m, 1H) 1.93 (br d, J=11.84 Hz, 1H) 1.27-1.51 (m, 5H)

LCMS (ESI+): m/z 450.2 (M+H)

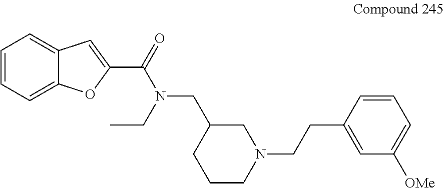 [SMILES: CCN(CC1CCCN(CCc2cccc(OC)c2)C1)C(=O)c1cc2ccccc2o1]

¹H NMR (400 MHz, TFA salt, METHANOL-d₄) δ ppm 7.74-7.72 (d, J=7.2 Hz, 1H) 7.60-7.58 (d, J=7.6 Hz, 1H) 7.48-7.44 (m, 2H) 7.35-7.32 (m, 1H) 7.28-7.21 (m, 1H) 6.88-6.77 (m, 3H) 3.75 (s, 3H) 3.66-3.56 (m, 4H) 3.38-3.34 (m, 2H) 3.04-2.84 (m, 4H) 2.50-2.38 (m, 2H) 2.06-1.82 (m, 4H) 1.40-1.26 (m, 4H)

LCMS (ESI+): m/z 421.2 (M+H)

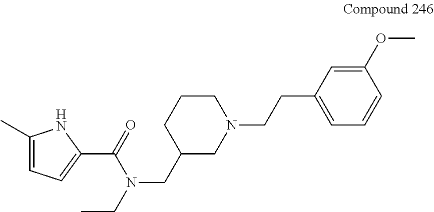

¹H NMR (400 MHz, METHANOL-d₄) δ ppm 7.19 (t, J=8.0 Hz, 1H) 6.85-6.75 (m, 3H) 6.64-6.47 (m, 1H) 5.98-5.88 (m, 1H) 4.16-3.81 (m, 1H) 3.75 (s, 3H) 3.70-3.44 (m, 4H) 3.38-3.32 (m, 1H) 3.29 (br. s., 1H) 3.22-3.11 (m, 1H) 3.05-2.97 (m, 2H) 2.95-2.69 (m, 2H) 2.37-2.21 (m, 4H) 2.07-1.63 (m, 3H) 1.35-1.29 (m, 1H) 1.26 (t, J=6.8 Hz, 3H)

LCMS (ESI+): m/z 384.2 (M+H)

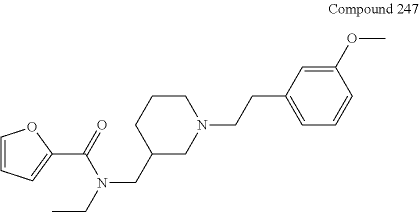 [SMILES: CCN(CC1CCCN(CCc2cccc(OC)c2)C1)C(=O)c1ccco1]

¹H NMR (400 MHz, METHANOL-d₄) δ ppm 7.75-7.69 (m, 1H) 7.22 (t, J=8.0 Hz, 1H) 7.10 (d, J=3.1 Hz, 1H) 6.89-6.77 (m, 3H) 6.64-6.58 (m, 1H) 3.78 (s, 3H) 3.74-3.45 (m, 5H) 3.35 (t, J=8.2 Hz, 3H) 3.15-2.75 (m, 4H) 2.50-2.25 (m, 1H) 2.09-1.74 (m, 3H) 1.41-1.16 (m, 4H)

LCMS (ESI+): m/z 371.2 (M+H)

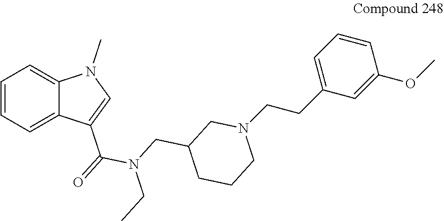 [SMILES: CCN(CC1CCCN(CCc2cccc(OC)c2)C1)C(=O)c1cn(C)c2ccccc12]

¹H NMR (400 MHz, METHANOL-d₄) δ ppm 7.72 (d, J=7.8 Hz, 1H) 7.57 (s, 1H) 7.43 (d, J=8.2 Hz, 1H) 7.27-7.14 (m, 3H) 6.84-6.76 (m, 3H) 3.87-3.81 (m, 3H) 3.77-3.70 (m, 3H) 3.68-3.59 (m, 3H) 3.58-3.41 (m, 3H) 3.28-3.20 (m, 2H) 3.08-2.92 (m, 2H) 2.82 (t, J=11.9 Hz, 1H) 2.73-2.29 (m, 2H) 2.03-1.76 (m, 3H) 1.32-1.15 (m, 4H)

LCMS (ESI+): m/z 434.2 (M+H)

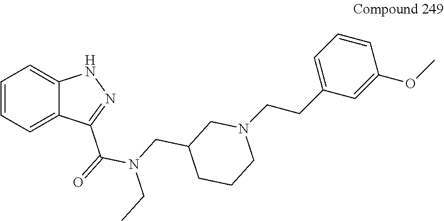 [SMILES: CCN(CC1CCCN(CCc2cccc(OC)c2)C1)C(=O)c1n[nH]c2ccccc12]

¹H NMR (400 MHz, METHANOL-d₄) δ ppm 8.17-7.90 (m, 1H) 7.59 (d, J=8.4 Hz, 1H) 7.42 (t, J=7.6 Hz, 1H) 7.21 (td, J=7.8, 15.4 Hz, 2H) 6.87-6.73 (m, 3H) 3.84 (br d, J=5.7 Hz, 1H) 3.74 (s, 3H) 3.64 (br s, 4H) 3.35 (br d, J=7.4 Hz, 2H) 3.15-2.64 (m, 4H) 2.42 (br s, 1H) 2.16-1.74 (m, 3H) 1.53-1.03 (m, 5H)

LCMS (ESI+): m/z 421.2 (M+H)

### Example 14: General Protocol K for Synthesis of Exemplary Compounds

General Protocol K to synthesize exemplary compounds of Formula (I) is described in Scheme 11 and the procedures set forth below.

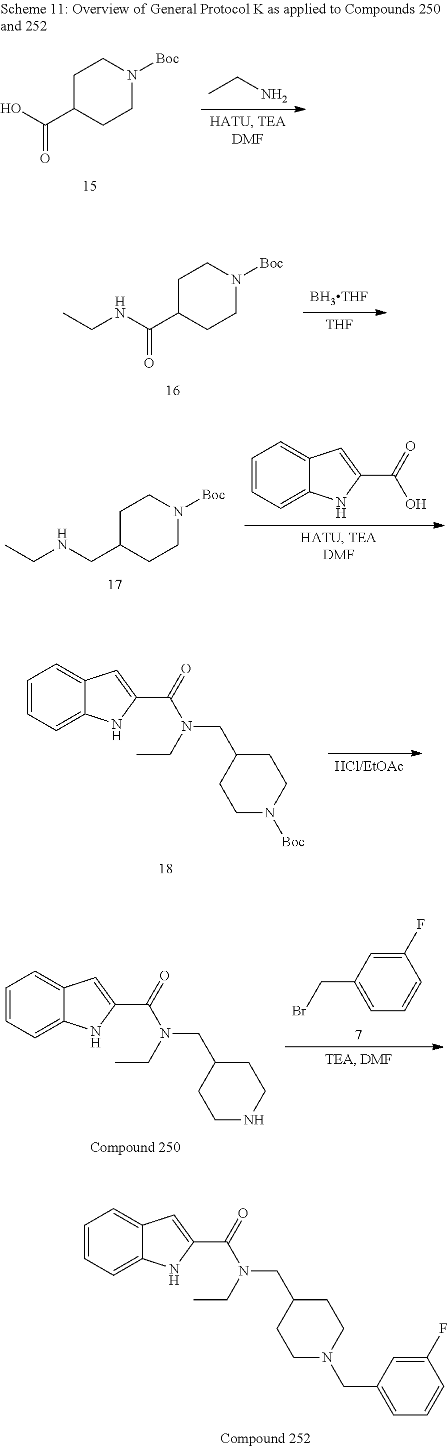

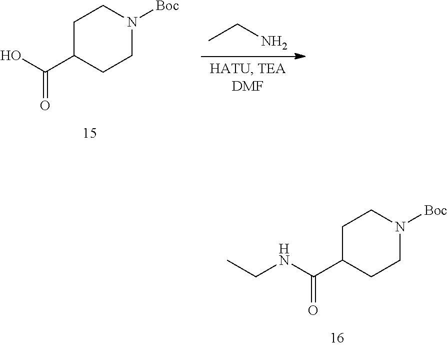

Procedure for the preparation of compound 16: A mixture of compound 15 (2.0 g, 8.7 mmol, 1.0 eq), ethanamine (1.4 g, 17.4 mmol, 2.0 eq, HCl salt), HATU (4.0 g, 10.5 mmol, 1.2 eq), and TEA (4.4 g, 43.6 mmol, 5.0 eq) in 20 mL of DMF was stirred at 15° C. for 16 hrs. LCMS showed the reactant was consumed completely. The reaction mixture was partitioned between 20 mL of ethyl acetate and 20 mL of water. The organic phase was separated, washed with four 20 mL portions of brine, dried over anhydrous Na₂SO₄, filtered and concentrated under reduced pressure to give 4.0 g of crude intermediate 16 as an orange oil. The crude product was used into the next step without further purification.

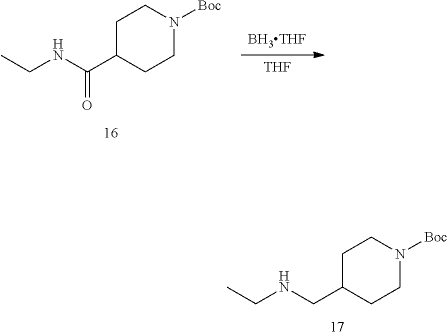 [SMILES: CCNC(=O)C1CCN(C(=O)OC(C)(C)C)CC1 ; CCNCC1CCN(C(=O)OC(C)(C)C)CC1 ; "BH3·THF" ; "THF"]

Procedure for the preparation of compound 17: To a solution of intermediate 16 (4.0 g, 15.6 mmol, 1.0 eq) in 50 mL of THF was added BH₃-THF (1 M, 46.8 mL, 3.0 eq) at 15° C. The mixture was allowed to stir at 60° C. for 16 hrs. LCMS analysis showed the reactant was consumed completely. The reaction mixture was quenched by addition of 50 mL of methanol at 60° C., and then concentrated under reduced pressure to give 4.7 g of compound 17 as a white gum. The crude product 17 was used into the next step without further purification.

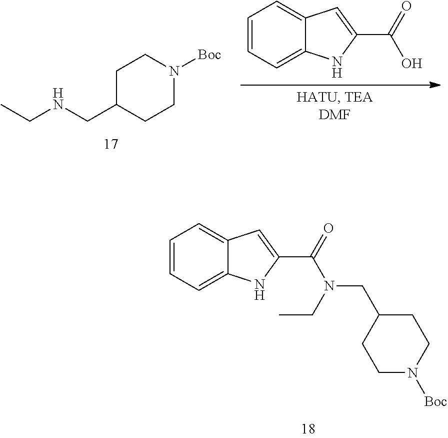

Procedure for the preparation of compound 18: To a solution of indole-2-carboxylic acid (1.6 g, 9.9 mmol, 1.2 eq) in 20 mL of DMF was added HATU (3.8 g, 9.9 mmol, 1.2 eq) and TEA (1.3 g, 12.4 mmol, 1.5 eq) at 15° C. The mixture was stirred at for 0.5 hr at the same temperature. Then compound 17 (2.0 g, 8.3 mmol, 1.0 eq) was added, the mixture was stirred at 15° C. for additional 15.5 hrs. LCMS analysis showed the reactant was consumed completely. The reaction mixture was partitioned between 20 mL of ethyl acetate and 20 mL of water. The organic phase was separated, washed with three 20 mL portions of brine, dried over anhydrous Na₂SO₄, filtered and concentrated under reduced pressure to give an oil. The residue was purified by column chromatography (SiO2 eluting with petroleum ether/ethyl acetate=20/1 to 2/1) to afford 600 mg of compound 18 (19% yield) as a light yellow solid.

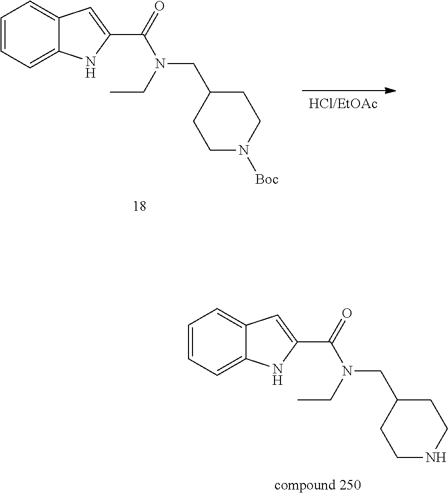 [SMILES: CCN(CC1CCN(C(=O)OC(C)(C)C)CC1)C(=O)c1cc2ccccc2[nH]1 ; "HCl/EtOAc" ; CCN(CC1CCNCC1)C(=O)c1cc2ccccc2[nH]1]

A mixture of compound 18 (600.0 mg, 1.6 mmol, 1.0 eq) in 10 mL of 4M HCl/ethyl acetate was stirred at 15° C. for 16 hrs. LCMS analysis showed the reactant was consumed completely. The reaction mixture was concentrated under reduced pressure to remove the solvent to afford a yellow solid. 70 mg of the residue was purified by prep-HPLC (TFA condition) to afford compound 250 (17.3 mg, 3.5% yield, as the HCl salt) as a white solid for delivery. And the other part of compound 250 (500.0 mg, crude) was used directly in the next step as a light yellow solid.

¹H NMR: (400 MHz, METHANOL-d₄) δ ppm 7.62 (d, J=8.4 Hz, 1H), 7.44 (d, J=7.9 Hz, 1H), 7.21 (t, J=7.5 Hz, 1H), 7.10-7.04 (m, 1H), 6.85 (br s, 1H), 3.86-3.50 (m, 4H), 3.39 (br d, J=11.5 Hz, 2H), 3.03-2.90 (m, 2H), 2.16 (br s, 1H), 1.94 (br d, J=13.7 Hz, 2H), 1.48 (br d, J=6.6 Hz, 2H), 1.31 (br t, J=7.1 Hz, 3H)

LCMS (ESI+): m/z 286.1 (M+H)

The following compounds were prepared analogously:

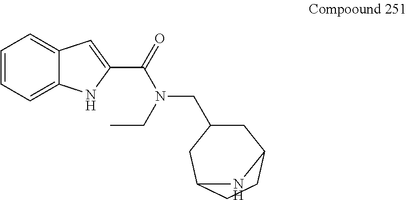 [SMILES: CCN(CC1CC2CCC(C1)N2)C(=O)c1cc2ccccc2[nH]1]

¹H NMR (400 MHz, METHANOL-d₄) 6=7.62 (d, J=8.2 Hz, 1H), 7.43 (d, J=8.2 Hz, 1H), 7.22 (t, J=7.6 Hz, 1H), 7.04-7.10 (m, 1H), 6.87 (s, 1H), 3.96 (br s, 2H), 3.85 (br d, J=6.2 Hz, 2H), 3.76 (br s, 2H), 2.44 (br s, 1H), 2.36-2.16 (m, 4H), 2.16-1.99 (m, 2H), 1.79 (br d, J=13.5 Hz, 2H), 1.29 (t, J=7.1 Hz, 3H).

LCMS (ESI+): m/z 312.1 (M+H)

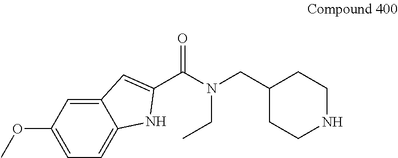 [SMILES: CCN(CC1CCNCC1)C(=O)c1cc2cc(OC)ccc2[nH]1]

¹H NMR (400 MHz, METHANOL-d₄) δ ppm 7.30 (d, J=8.8 Hz, 1H) 7.07 (d, J=1.8 Hz, 1H) 6.86 (dd, J=2.2, 8.8 Hz, 1H) 6.76 (br. s., 1H) 3.81-3.67 (m, 5H) 3.54 (br. s., 2H) 3.37 (d, J=11.9 Hz, 2H) 2.94 (t, J=11.9 Hz, 2H) 2.13 (br. s., 1H) 1.91 (d, J=13.7 Hz, 2H) 1.45 (br. s., 2H) 1.29 (t, J=6.8 Hz, 3H)

LCMS (ESI+): m/z 316.1 (M+H)

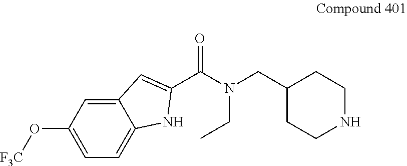 [SMILES: CCN(CC1CCNCC1)C(=O)c1cc2cc(OC(F)(F)F)ccc2[nH]1]

¹H NMR (400 MHz, METHANOL-d₄) δ ppm 7.54-7.46 (m, 2H) 7.12 (dd, J=1.3, 8.8 Hz, 1H) 6.89 (br s, 1H) 3.74 (br s, 2H) 3.57 (br s, 2H) 3.39 (br d, J=9.9 Hz, 2H) 2.97 (br t, J=11.9 Hz, 2H) 2.15 (br d, J=5.7 Hz, 1H) 1.94 (br d, J=12.6 Hz, 2H) 1.50 (br s, 2H) 1.30 (t, J=7.1 Hz, 3H)

LCMS (ESI+): m/z 370.1 (M+H)

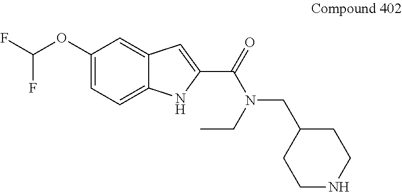 [SMILES: CCN(CC1CCNCC1)C(=O)c1cc2cc(OC(F)F)ccc2[nH]1]

¹H NMR (400 MHz, METHANOL-d₄) δ ppm 7.37-7.49 (m, 2H) 7.05 (dd, J=9.21, 2.19 Hz, 1H) 6.88 (br d, J=19.73 Hz, 1H) 6.51-6.75 (m, 1H) 3.48-3.86 (m, 4H) 3.34-3.43 (m, 2H) 2.96 (br t, J=12.94 Hz, 2H) 2.16 (br s, 1H) 1.94 (s, 2H) 1.39-1.57 (m, 1H) 1.24-1.37 (m, 5H)

LCMS (ESI+): m/z 352.1 (M+H)

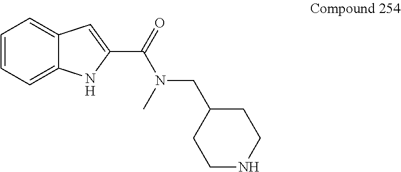 [SMILES: CN(CC1CCNCC1)C(=O)c1cc2ccccc2[nH]1]

¹H NMR: (400 MHz, METHANOL-d₄) δ ppm 7.62 (d, J=7.9 Hz, 1H) 7.43 (d, J=8.3 Hz, 1H) 7.22 (t, J=7.7 Hz, 1H) 7.09-7.03 (m, 1H) 6.97-6.87 (m, 1H) 3.58 (br. s., 2H) 3.43 (br. s., 5H) 2.98 (br. s., 2H) 2.16 (br. s., 1H) 1.94 (d, J=11.4 Hz, 2H) 1.51 (br. s., 2H)

LCMS (ESI+): m/z 272.1 (M+H)

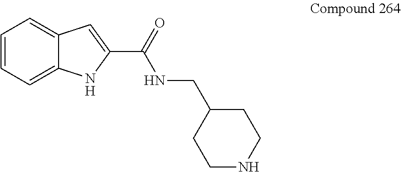 [SMILES: O=C(NCC1CCNCC1)c1cc2ccccc2[nH]1]

¹H NMR: (400 MHz, METHANOL-d₄) δ ppm 7.59 (d, J=8.16 Hz, 1H) 7.43 (d, J=8.38 Hz, 1H) 7.21 (t, J=7.61 Hz, 1H) 7.03-7.09 (m, 2H) 3.42 (br d, J=13.23 Hz, 2H) 3.35 (d, J=6.62 Hz, 2H) 2.95-3.03 (m, 2H) 1.97-2.06 (m, 3H) 1.43-1.55 (m, 2H)

LCMS (ESI+): m/z 258.1 (M+H)

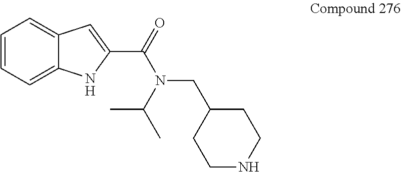 [SMILES: CC(C)N(CC1CCNCC1)C(=O)c1cc2ccccc2[nH]1]

¹H NMR: (400 MHz, METHANOL-d₄) δ ppm 7.61 (d, J=8.38 Hz, 1H) 7.43 (d, J=7.94 Hz, 1H) 7.21 (t, J=7.72 Hz, 1H) 7.04-7.11 (m, 1H) 6.79 (s, 1H) 3.40 (d, J=13.67 Hz, 4H) 2.96 (t, J=11.91 Hz, 2H) 2.18 (br. s., 2H) 2.00 (d, J=14.11 Hz, 2H) 1.46 (d, J=8.82 Hz, 2H) 1.32 (d, J=6.62 Hz, 6H)

LCMS (ESI+): m/z 300.1 (M+H)

### Example 15: General Protocol L for Synthesis of Exemplary Compounds

General Protocol L to synthesize exemplary compounds of Formula (I) is described in Scheme 12 and the procedures set forth below.

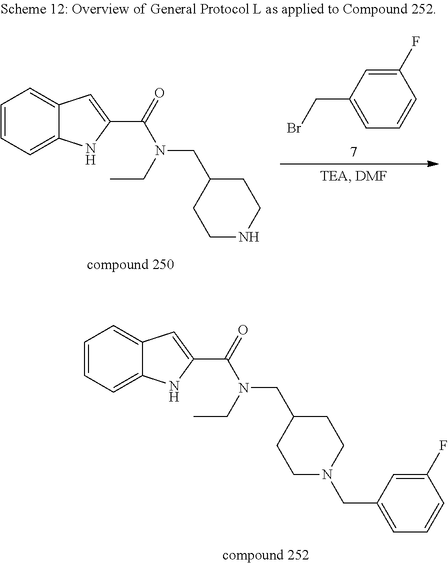

Procedure for the preparation of compound 252: A mixture of compound 250 (30.0 mg, 93.2 μmol, 1.0 eq, HCl), 3-fluoro-benzyl bromide (17.6 mg, 93.2 μmol, 1.0 eq), and TEA (28.3 mg, 279.6 μmol, 3.0 eq) in 1 mL of DMF was stirred at 15° C. for 2 hrs. LCMS analysis showed the reactant was consumed completely. The reaction mixture was filtered and the filtrate was purified by prep-HPLC (TFA condition) to afford 15.3 mg (32%) of the TFA salt of compound 252 as a white solid.

¹H NMR: (400 MHz, METHANOL-d₄) δ ppm 7.61 (br d, J=7.9 Hz, 1H), 7.55-7.48 (m, 1H), 7.43 (br d, J=7.9 Hz, 1H), 7.35-7.19 (m, 4H), 7.10-7.04 (m, 1H), 6.85 (br s, 1H), 4.30 (s, 2H), 3.77 (br s, 2H), 3.51 (br d, J=11.5 Hz, 4H), 3.00 (br t, J=12.3 Hz, 2H), 2.21-1.91 (m, 3H), 1.52 (br s, 2H), 1.31 (br t, J=7.1 Hz, 3H)

LCMS (ESI+): m/z 394.1 (M+H)

The following compounds were prepared analogously using General Protocol L:

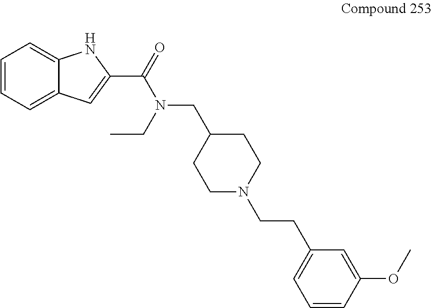 [SMILES: CCN(CC1CCN(CCc2cccc(OC)c2)CC1)C(=O)c1cc2ccccc2[nH]1]

¹H NMR: (400 MHz, METHANOL-d₄) δ 7.60 (d, J=7.94 Hz, 1H) 7.42 (d, J=7.94 Hz, 1H) 7.16-7.27 (m, 2H) 7.05 (t, J=7.50 Hz, 1H) 6.77-6.90 (m, 4H) 3.49-3.86 (m, 9H) 3.29-3.47 (m, 4H) 2.91-3.05 (m, 4H) 2.15 (br. s., 1H) 1.89-2.07 (m, 2H) 1.31 (t, J=6.84 Hz, 3H)

LCMS (ESI+): m/z 420.2 (M+H)

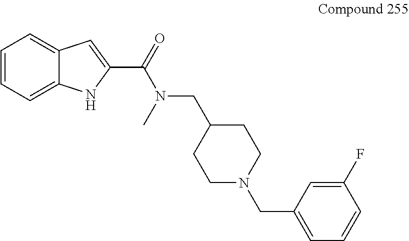 [SMILES: CN(CC1CCN(Cc2cccc(F)c2)CC1)C(=O)c1cc2ccccc2[nH]1]

¹H NMR: (400 MHz, METHANOL-d₄) δ ppm 7.60 (d, J=7.9 Hz, 1H) 7.52-7.45 (m, 1H) 7.41 (d, J=7.9 Hz, 1H) 7.31 (d, J=6.2 Hz, 2H) 7.25-7.17 (m, 2H) 7.07-7.01 (m, 1H) 6.94-6.87 (m, 1H) 4.29 (br. s., 2H) 3.62-3.32 (m, 7H) 2.99 (t, J=12.1 Hz, 2H) 2.13 (d, J=3.5 Hz, 1H) 1.95 (d, J=11.5 Hz, 2H) 1.54 (br. s., 2H)

LCMS (ESI+): m/z 380.2 (M+H)

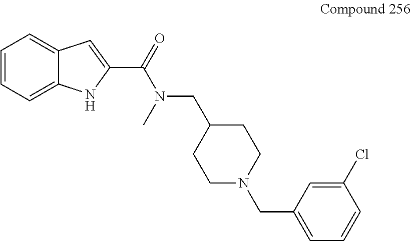 [SMILES: CN(CC1CCN(Cc2cccc(Cl)c2)CC1)C(=O)c1cc2ccccc2[nH]1]

¹H NMR: (400 MHz, METHANOL-d₄) δ ppm 7.60 (d, J=7.9 Hz, 1H) 7.55 (br. s., 1H) 7.52-7.48 (m, 1H) 7.47-7.44 (m, 1H) 7.41 (d, J=7.9 Hz, 2H) 7.20 (t, J=7.7 Hz, 1H) 7.04 (t, J=7.5 Hz, 1H) 6.91 (br. s., 1H) 4.28 (br. s., 2H) 3.61-3.33 (m, 7H) 3.04-2.95 (m, 2H) 2.12 (br. s., 1H) 1.97 (d, J=12.8 Hz, 2H) 1.52 (br. s., 2H)

LCMS (ESI+): m/z 396.1 (M+H)

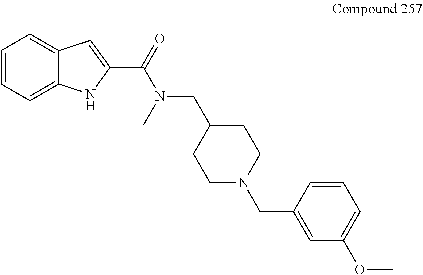 [SMILES: COc1cccc(CN2CCC(CN(C)C(=O)c3cc4ccccc4[nH]3)CC2)c1]

¹H NMR: (400 MHz, METHANOL-d₄) δ ppm 7.60 (d, J=7.9 Hz, 1H) 7.44-7.34 (m, 2H) 7.20 (t, J=7.5 Hz, 1H) 7.07-6.97 (m, 4H) 6.92 (d, J=10.1 Hz, 1H) 4.23 (br. s., 2H) 3.81 (s, 3H) 3.62-3.32 (m, 7H) 2.97 (t, J=12.1 Hz, 2H) 2.12 (br. s., 1H) 1.96 (d, J=12.8 Hz, 2H) 1.52 (br. s., 2H)

LCMS (ESI+): m/z 392.1 (M+H)

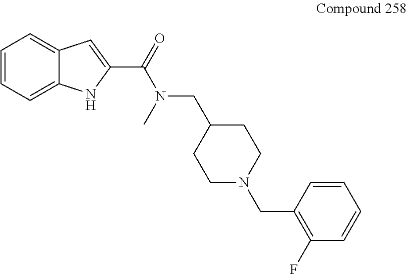 [SMILES: CN(CC1CCN(Cc2ccccc2F)CC1)C(=O)c1cc2ccccc2[nH]1]

¹H NMR: (400 MHz, METHANOL-d₄) δ ppm 7.60 (d, J=7.9 Hz, 1H) 7.57-7.50 (m, 2H) 7.41 (d, J=8.4 Hz, 1H) 7.32-7.18 (m, 3H) 7.04 (t, J=7.5 Hz, 1H) 6.91 (br. s., 1H) 4.36 (br. s., 2H) 3.54 (d, J=7.9 Hz, 4H) 3.40 (br. s., 3H) 3.06 (t, J=12.3 Hz, 2H) 2.13 (br. s., 1H) 1.97 (d, J=12.8 Hz, 2H) 1.54 (br. s., 2H)

LCMS (ESI+): m/z 380.1 (M+H)

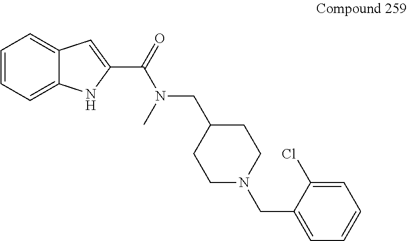 [SMILES: CN(CC1CCN(Cc2ccccc2Cl)CC1)C(=O)c1cc2ccccc2[nH]1]

¹H NMR: (400 MHz, METHANOL-d₄) δ ppm 7.63-7.54 (m, 3H) 7.51-7.39 (m, 3H) 7.20 (t, J=7.7 Hz, 1H) 7.04 (t, J=7.5 Hz, 1H) 6.92 (br. s., 1H) 4.46 (br. s., 2H) 3.55 (br. s., 4H) 3.47-3.33 (m, 3H) 3.15 (t, J=12.6 Hz, 2H) 2.15 (br. s., 1H) 1.97 (d, J=12.3 Hz, 2H) 1.56 (br. s., 2H)

LCMS (ESI+): m/z 396.1 (M+H)

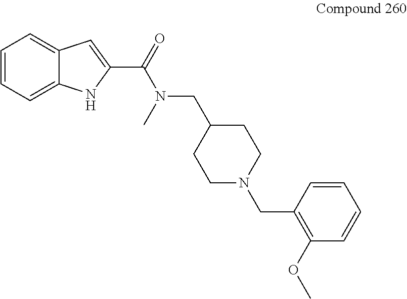 [SMILES: COc1ccccc1CN1CCC(CN(C)C(=O)c2cc3ccccc3[nH]2)CC1]

¹H NMR: (400 MHz, METHANOL-d₄) δ ppm 7.60 (d, J=8.4 Hz, 1H) 7.49-7.44 (m, 1H) 7.43-7.36 (m, 2H) 7.23-7.17 (m, 1H) 7.13-6.99 (m, 3H) 6.93 (d, J=7.1 Hz, 1H) 4.27 (br. s., 2H) 3.89 (s, 3H) 3.50 (d, J=11.5 Hz, 4H) 3.40 (br. s., 3H) 3.01 (t, J=12.3 Hz, 2H) 2.11 (d, J=3.5 Hz, 1H) 1.92 (br. s., 2H) 1.52 (br. s., 2H)

LCMS (ESI+): m/z 392.1 (M+H)

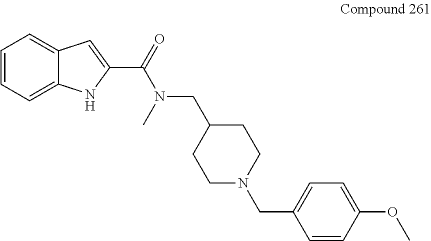 [SMILES: COc1ccc(CN2CCC(CN(C)C(=O)c3cc4ccccc4[nH]3)CC2)cc1]

¹H NMR: (400 MHz, METHANOL-d₄) δ ppm 7.59 (d, J=7.9 Hz, 1H) 7.39 (dd, J=8.2, 14.3 Hz, 3H) 7.20 (t, J=7.5 Hz, 1H) 7.04 (t, J=7.5 Hz, 1H) 6.99 (d, J=7.9 Hz, 2H) 6.92 (d, J=10.6 Hz, 1H) 4.19 (br. s., 2H) 3.79 (s, 3H) 3.57-3.34 (m, 7H) 2.92 (t, J=12.3 Hz, 2H) 2.10 (br. s., 1H) 1.94 (d, J=12.3 Hz, 2H) 1.50 (br. s., 2H)

LCMS (ESI+): m/z 392.2 (M+H)

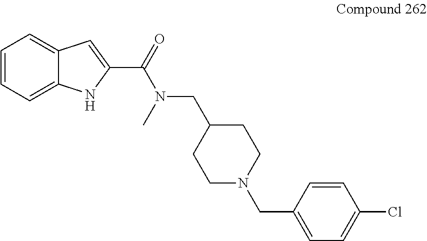 [SMILES: CN(CC1CCN(Cc2ccc(Cl)cc2)CC1)C(=O)c1cc2ccccc2[nH]1]

¹H NMR: (400 MHz, METHANOL-d₄) δ ppm 7.60 (d, J=7.9 Hz, 1H) 7.55-7.39 (m, 5H) 7.20 (t, J=7.5 Hz, 1H) 7.04 (t, J=7.5 Hz, 1H) 6.89 (d, J=14.6 Hz, 1H) 4.26 (br. s., 2H) 3.70-3.31 (m, 7H) 2.97 (t, J=12.3 Hz, 2H) 2.11 (br. s., 1H) 1.96 (d, J=11.9 Hz, 2H) 1.51 (br. s., 2H)

LCMS (ESI+): m/z 396.1 (M+H)

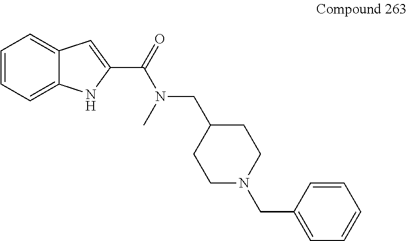 [SMILES: CN(CC1CCN(Cc2ccccc2)CC1)C(=O)c1cc2ccccc2[nH]1]

¹H NMR: (400 MHz, METHANOL-d₄) δ ppm 7.60 (d, J=7.9 Hz, 1H) 7.47 (s, 5H) 7.41 (d, J=8.4 Hz, 1H) 7.20 (t, J=7.7 Hz, 1H) 7.07-7.01 (m, 1H) 6.92 (d, J=9.7 Hz, 1H) 4.27 (br. s., 2H) 3.60-3.34 (m, 7H) 2.98 (t, J=12.3 Hz, 2H) 2.12 (br. s., 1H) 1.96 (d, J=12.8 Hz, 2H) 1.52 (br. s., 2H)

LCMS (ESI+): m/z 362.1 (M+H)

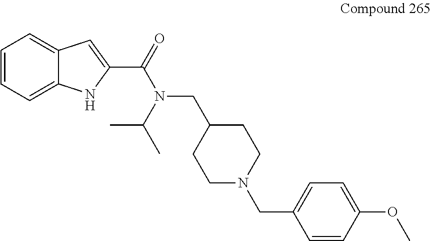 [SMILES: COc1ccc(CN2CCC(CN(C(=O)c3cc4ccccc4[nH]3)C(C)C)CC2)cc1]

¹H NMR: (400 MHz, METHANOL-d₄) δ ppm 7.60 (d, J=7.94 Hz, 1H) 7.41 (dd, J=11.47, 8.82 Hz, 3H) 7.21 (t, J=7.72 Hz, 1H) 6.97-7.12 (m, 3H) 6.78 (s, 1H) 4.20 (s, 2H) 3.82 (s, 3H) 3.49 (d, J=11.91 Hz, 2H) 3.41 (br. s., 2H) 2.93 (t, J=13.23 Hz, 2H) 2.14 (br. s., 1H) 2.02 (d, J=13.67 Hz, 2H) 1.92 (d, J=15.44 Hz, 1H) 1.38-1.57 (m, 2H) 1.25-1.35 (m, 6H)

LCMS (ESI+): m/z 420.2 (M+H)

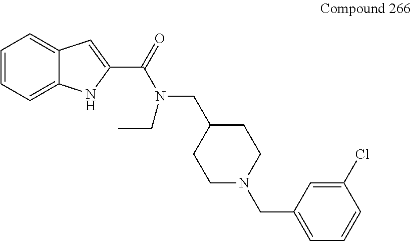 [SMILES: CCN(CC1CCN(Cc2cccc(Cl)c2)CC1)C(=O)c1cc2ccccc2[nH]1]

¹H NMR: (400 MHz, METHANOL-d₄) δ ppm 7.67-7.38 (m, 6H), 7.21 (br t, J=7.7 Hz, 1H), 7.11-7.02 (m, 1H), 6.91-6.80 (m, 1H), 4.34-4.22 (m, 2H), 3.76 (br s, 2H), 3.67-3.36 (m, 4H), 3.05-2.91 (m, 2H), 2.23-1.82 (m, 3H), 1.62-1.35 (m, 2H), 1.31 (br t, J=6.8 Hz, 3H)

LCMS (ESI+): m/z 410.1 (M+H)

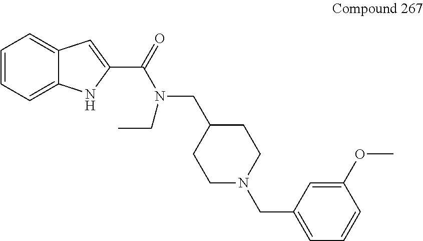 [SMILES: CCN(CC1CCN(Cc2cccc(OC)c2)CC1)C(=O)c1cc2ccccc2[nH]1]

¹H NMR: (400 MHz, METHANOL-d₄) δ ppm 7.61 (br d, J=7.9 Hz, 1H), 7.46-7.35 (m, 2H), 7.21 (t, J=7.5 Hz, 1H), 7.10-7.00 (m, 4H), 6.90-6.80 (m, 1H), 4.24 (s, 2H), 3.82 (s, 3H), 3.76 (br s, 2H), 3.66-3.43 (m, 4H), 3.04-2.91 (m, 2H), 2.22-1.86 (m, 3H), 1.65-1.37 (m, 2H), 1.31 (br t, J=7.1 Hz, 3H)

LCMS (ESI+): m/z 406.2 (M+H)

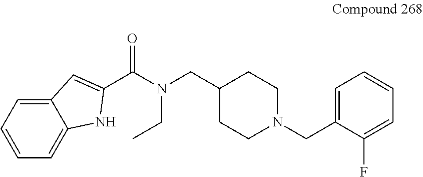 [SMILES: CCN(CC1CCN(Cc2ccccc2F)CC1)C(=O)c1cc2ccccc2[nH]1]

¹H NMR: (400 MHz, METHANOL-d₄) δ ppm 7.67-7.50 (m, 3H), 7.43 (br d, J=8.4 Hz, 1H), 7.36-7.16 (m, 3H), 7.11-7.02 (m, 1H), 6.90-6.79 (m, 1H), 4.37 (br s, 2H), 3.76 (br s, 2H), 3.68-3.32 (m, 4H), 3.16-2.98 (m, 2H), 1.99 (br d, J=13.7 Hz, 3H), 1.60-1.24 (m, 5H)

LCMS (ESI+): m/z 394.1 (M+H)

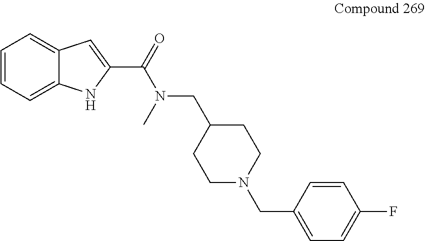 [SMILES: CN(CC1CCN(Cc2ccc(F)cc2)CC1)C(=O)c1cc2ccccc2[nH]1]

¹H NMR: (400 MHz, METHANOL-d₄) δ ppm 7.59 (d, J=7.9 Hz, 1H) 7.49 (d, J=12.3 Hz, 2H) 7.41 (d, J=7.9 Hz, 1H) 7.20 (d, J=3.5 Hz, 3H) 7.04 (t, J=7.5 Hz, 1H) 6.89 (d, J=12.8 Hz, 1H) 4.27 (br. s., 2H) 3.33-3.69 (m, 7H) 2.97 (t, J=12.6 Hz, 2H) 2.12 (br. s., 1H) 1.89-2.02 (m, 2H) 1.50 ppm (br. s., 2H)

LCMS (ESI+): m/z 380.1 (M+H)

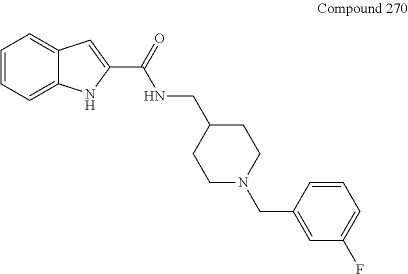 [SMILES: O=C(NCC1CCN(Cc2cccc(F)c2)CC1)c1cc2ccccc2[nH]1]

¹H NMR: (400 MHz, METHANOL-d₄) δ ppm 7.57 (d, J=8.16 Hz, 1H) 7.47-7.53 (m, 1H) 7.41 (d, J=8.16 Hz, 1H) 7.23-7.31 (m, 3H) 7.17-7.21 (m, 1H) 7.01-7.07 (m, 1H) 7.01-7.07 (m, 1H) 4.30 (s, 2H) 3.51 (br d, J=11.91 Hz, 2H) 3.32 (d, J=6.61 Hz, 2H) 2.95-3.05 (m, 2H) 2.04 (br d, J=15.44 Hz, 2H) 1.95 (br s, 1H) 1.43-1.56 (m, 2H)

LCMS (ESI+): m/z 366.2 (M+H)

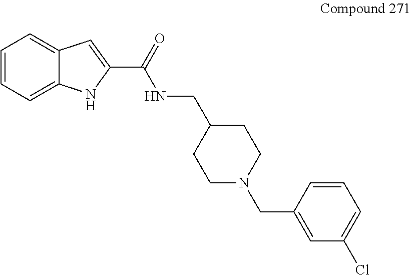 [SMILES: O=C(NCC1CCN(Cc2cccc(Cl)c2)CC1)c1cc2ccccc2[nH]1]

¹H NMR: (400 MHz, METHANOL-d₄) δ ppm 7.53-7.59 (m, 2H) 7.37-7.50 (m, 4H) 7.19 (br s, 1H) 7.02 (br s, 2H) 4.28 (br s, 2H) 3.50 (br d, J=10.36 Hz, 2H) 3.31-3.36 (m, 2H) 2.99 (br t, J=13.89 Hz, 2H) 2.03 (br d, J=14.77 Hz, 2H) 1.94 (br s, 1H) 1.49 (br d, J=13.67 Hz, 2H)

LCMS (ESI+): m/z 382.1 (M+H)

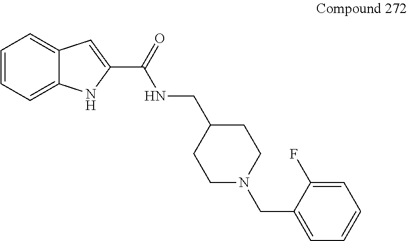 [SMILES: O=C(NCC1CCN(Cc2ccccc2F)CC1)c1cc2ccccc2[nH]1]

¹H NMR: (400 MHz, METHANOL-d₄) δ ppm 7.51-7.59 (m, 3H) 7.41 (d, J=8.16 Hz, 1H) 7.23-7.33 (m, 2H) 7.19 (t, J=7.61 Hz, 1H) 7.00-7.06 (m, 2H) 4.36 (s, 2H) 3.55 (br d, J=13.23 Hz, 2H) 3.32-3.37 (m, 2H) 3.00-3.12 (m, 2H) 2.04 (br d, J=13.89 Hz, 2H) 1.95 (br s, 1H) 1.43-1.58 (m, 2H)

LCMS (ESI+): m/z 366.2 (M+H)

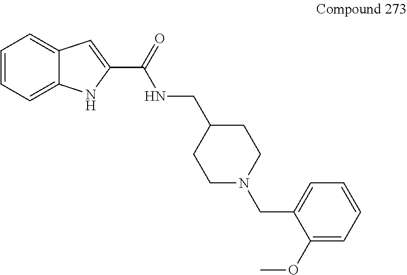 [SMILES: COc1ccccc1CN1CCC(CNC(=O)c2cc3ccccc3[nH]2)CC1]

¹H NMR: (400 MHz, METHANOL-d₄) δ ppm 7.64 (d, J=8.33 Hz, 1H) 7.38-7.46 (m, 4H) 7.18 (s, 1H) 7.10 (t, J=7.45 Hz, 1H) 7.04 (t, J=7.24 Hz, 1H) 6.96 (d, J=8.77 Hz, 1H) 4.21 (br s, 2H) 3.87 (s, 3H) 3.53 (br s, 2H) 2.74 (br d, J=10.96 Hz, 2H) 2.41 (br d, J=13.59 Hz, 2H) 2.02 (br s, 3H) 1.82 (br d, J=14.91 Hz, 2H)

LCMS (ESI+): m/z 378.1 (M+H)

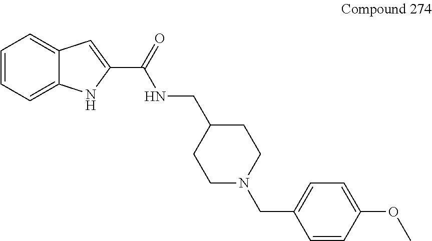 [SMILES: COc1ccc(CN2CCC(CNC(=O)c3cc4ccccc4[nH]3)CC2)cc1]

¹H NMR: (400 MHz, METHANOL-d₄) δ ppm 7.65 (d, J=8.33 Hz, 1H) 7.44 (s, 1H) 7.37 (br d, J=8.33 Hz, 2H) 7.08-7.17 (m, 3H) 6.95 (br d, J=7.89 Hz, 2H) 4.08 (br s, 2H) 3.83 (s, 3H) 3.54 (br s, 2H) 2.67 (br s, 2H) 2.37 (s, 2H) 2.02-2.09 (m, 1H) 1.86 (br d, J=9.65 Hz, 4H)

LCMS (ESI+): m/z 378.1 (M+H)

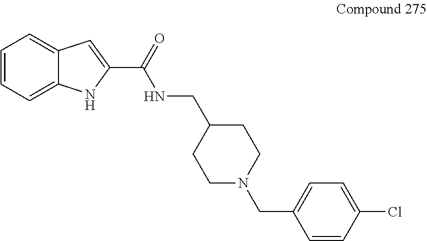 [SMILES: O=C(NCC1CCN(Cc2ccc(Cl)cc2)CC1)c1cc2ccccc2[nH]1]

¹H NMR: (400 MHz, METHANOL-d₄) δ ppm 7.58 (br d, J=8.60 Hz, 1H) 7.36-7.46 (m, 5H) 7.20 (br t, J=7.28 Hz, 1H) 7.05 (br s, 2H) 3.91 (br s, 2H) 3.32-3.34 (m, 2H) 3.32-3.34 (m, 2H) 3.20 (br s, 2H) 2.54 (br s, 2H) 1.76-1.95 (m, 3H) 1.38-1.52 (m, 2H)

LCMS (ESI+): m/z 382.1 (M+H)

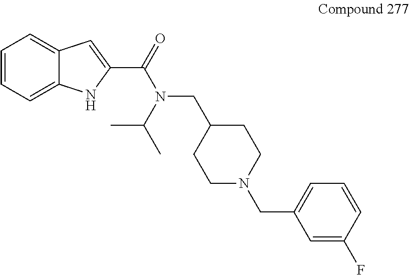 [SMILES: CC(C)N(CC1CCN(Cc2cccc(F)c2)CC1)C(=O)c1cc2ccccc2[nH]1]

¹H NMR: (400 MHz, METHANOL-d₄) δ ppm 7.60 (d, J=7.94 Hz, 1H) 7.42 (d, J=8.38 Hz, 1H) 7.28-7.35 (m, 1H) 7.20 (t, J=7.28 Hz, 1H) 6.95-7.14 (m, 4H) 6.74 (s, 1H) 3.50 (br. s., 2H) 3.37 (br. s., 2H) 2.88 (br. s., 2H) 1.91-2.08 (m, 3H) 1.82-1.91 (m, 1H) 1.74 (d, J=12.79 Hz, 2H) 1.30 (d, J=6.17 Hz, 8H)

LCMS (ESI+): m/z 408.3 (M+H)

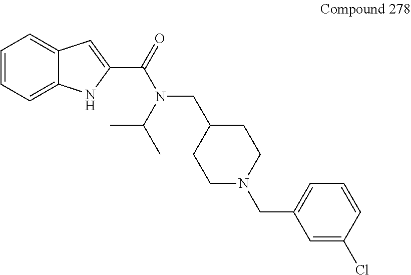 [SMILES: CC(C)N(CC1CCN(Cc2cccc(Cl)c2)CC1)C(=O)c1cc2ccccc2[nH]1]

¹H NMR: (400 MHz, METHANOL-d₄) δ ppm 7.60 (d, J=7.94 Hz, 1H) 7.42 (d, J=8.38 Hz, 1H) 7.35 (br. s., 1H) 7.14-7.30 (m, 4H) 7.06 (t, J=7.50 Hz, 1H) 6.74 (s, 1H) 3.48 (br. s., 2H) 3.36 (d, J=10.14 Hz, 2H) 2.88 (br. s., 2H) 1.99 (t, J=11.03 Hz, 3H) 1.89 (br. s., 1H) 1.75 (d, J=11.91 Hz, 2H) 1.30 (d, J=6.17 Hz, 8H)

LCMS (ESI+): m/z 424.1 (M+H)

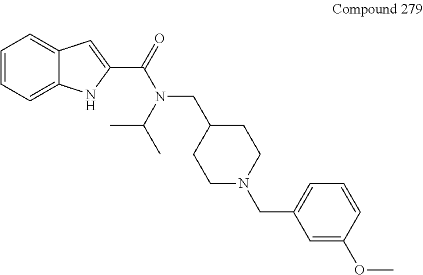 [SMILES: COc1cccc(CN2CCC(CN(C(=O)c3cc4ccccc4[nH]3)C(C)C)CC2)c1]

¹H NMR: (400 MHz, METHANOL-d₄) δ ppm 7.57-7.63 (m, 1H) 7.42 (d, J=7.94 Hz, 1H) 7.16-7.25 (m, 2H) 7.02-7.09 (m, 1H) 6.80-6.93 (m, 3H) 6.74 (s, 1H) 3.78 (s, 3H) 3.51 (br. s., 2H) 3.36 (d, J=9.70 Hz, 2H) 2.95 (br. s., 2H) 2.02 (d, J=8.82 Hz, 2H) 1.75 (d, J=11.47 Hz, 3H) 1.30 (d, J=6.17 Hz, 8H)

LCMS (ESI+): m/z 420.3 (M+H)

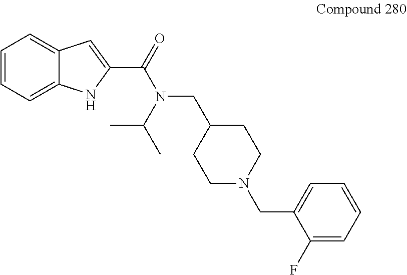 [SMILES: CC(C)N(CC1CCN(Cc2ccccc2F)CC1)C(=O)c1cc2ccccc2[nH]1]

¹H NMR: (400 MHz, METHANOL-d₄) δ ppm 7.59 (d, J=8.38 Hz, 1H) 7.34-7.44 (m, 2H) 7.26-7.33 (m, 1H) 7.11-7.23 (m, 2H) 7.06 (q, J=7.50 Hz, 2H) 6.73 (s, 1H) 3.59 (br. s., 2H) 3.36 (d, J=10.58 Hz, 2H) 2.86-3.02 (m, 2H) 1.99-2.13 (m, 2H) 1.85 (br. s., 1H) 1.74 (d, J=12.35 Hz, 2H) 1.11-1.43 (m, 8H)

LCMS (ESI+): m/z 408.2 (M+H)

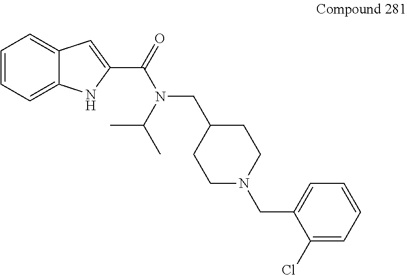 [SMILES: CC(C)N(CC1CCN(Cc2ccccc2Cl)CC1)C(=O)c1cc2ccccc2[nH]1]

¹H NMR: (400 MHz, METHANOL-d₄) δ ppm 7.59 (d, J=7.94 Hz, 1H) 7.40-7.49 (m, 2H) 7.37 (d, J=7.06 Hz, 1H) 7.15-7.31 (m, 3H) 7.01-7.09 (m, 1H) 6.74 (s, 1H) 3.62 (s, 2H) 3.37 (br. s., 2H) 2.93 (br. s., 2H) 1.98-2.13 (m, 2H) 1.88 (br. s., 1H) 1.73 (d, J=11.91 Hz, 2H) 1.20-1.39 (m, 8H)

LCMS (ESI+): m/z 424.2 (M+H)

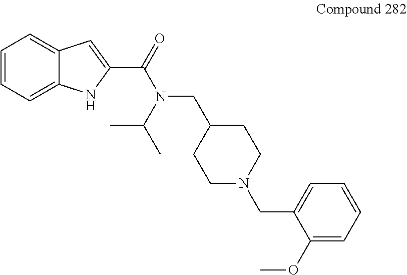 [SMILES: COc1ccccc1CN1CCC(CN(C(=O)c2cc3ccccc3[nH]2)C(C)C)CC1]

¹H NMR: (400 MHz, METHANOL-d₄) δ ppm 7.60 (d, J=7.94 Hz, 1H) 7.42 (d, J=8.38 Hz, 1H) 7.29 (d, J=7.06 Hz, 2H) 7.20 (t, J=7.72 Hz, 1H) 7.06 (t, J=7.28 Hz, 1H) 6.89-7.02 (m, 2H) 6.74 (s, 1H) 3.83 (s, 3H) 3.72 (br. s., 2H) 3.36 (br. s., 2H) 3.06 (br. s., 3H) 2.16-2.31 (m, 2H) 1.88-2.01 (m, 1H) 1.77 (br. s., 2H) 1.30 (d, J=6.62 Hz, 8H)

LCMS (ESI+): m/z 420.2 (M+H)

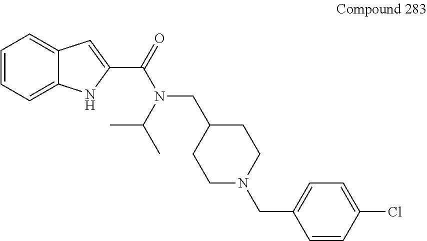 [SMILES: CC(C)N(CC1CCN(Cc2ccc(Cl)cc2)CC1)C(=O)c1cc2ccccc2[nH]1]

¹H NMR: (400 MHz, METHANOL-d₄) δ ppm 7.60 (d, J=8.38 Hz, 1H) 7.42 (d, J=7.94 Hz, 1H) 7.32 (br. s., 4H) 7.17-7.23 (m, 1H) 7.03-7.09 (m, 1H) 6.74 (s, 1H) 3.56 (br. s., 2H) 3.37 (br. s., 2H) 2.94 (br. s., 2H) 2.08 (br. s., 2H) 1.90 (br. s., 1H) 1.77 (d, J=12.35 Hz, 2H) 1.20-1.37 (m, 8H)

LCMS (ESI+): m/z 424.1 (M+H)

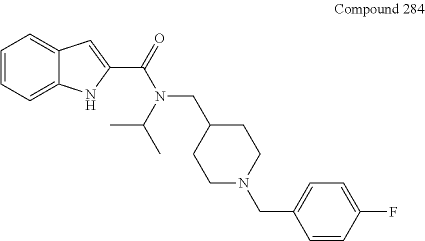 [SMILES: CC(C)N(CC1CCN(Cc2ccc(F)cc2)CC1)C(=O)c1cc2ccccc2[nH]1]

¹H NMR: (400 MHz, METHANOL-d₄) δ ppm 7.60 (d, J=7.50 Hz, 1H) 7.52 (br. s., 2H) 7.43 (d, J=7.94 Hz, 1H) 7.17-7.29 (m, 3H) 7.07 (d, J=6.62 Hz, 1H) 6.78 (br. s., 1H) 4.26 (br. s., 2H) 3.34-3.54 (m, 4H) 2.94 (t, J=12.57 Hz, 2H) 2.15 (br. s., 1H) 2.02 (d, J=14.11 Hz, 2H) 1.91 (br. s., 1H) 1.49 (d, J=7.94 Hz, 2H) 1.30 (d, J=5.29 Hz, 6H)

LCMS (ESI+): m/z 408.2 (M+H)

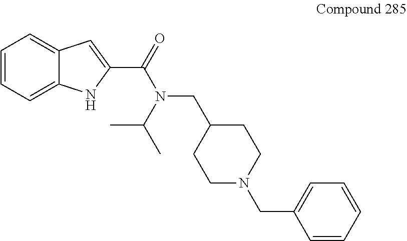 [SMILES: CC(C)N(CC1CCN(Cc2ccccc2)CC1)C(=O)c1cc2ccccc2[nH]1]

¹H NMR: (400 MHz, METHANOL-d₄) δ ppm 7.60 (d, J=8.38 Hz, 1H) 7.42 (d, J=8.38 Hz, 1H) 7.25-7.36 (m, 5H) 7.20 (t, J=7.72 Hz, 1H) 7.02-7.09 (m, 1H) 6.74 (s, 1H) 3.54 (br. s., 2H) 3.37 (br. s., 2H) 2.94 (br. s., 2H) 2.02 (d, J=7.94 Hz, 2H) 1.92 (d, J=11.47 Hz, 1H) 1.75 (d, J=12.35 Hz, 2H) 1.30 (d, J=6.17 Hz, 8H)

LCMS (ESI+): m/z 390.3 (M+H)

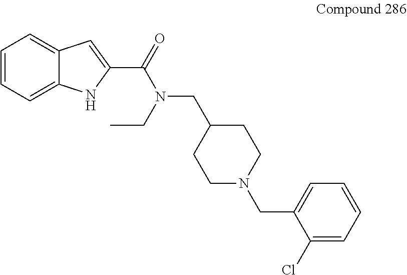 [SMILES: CCN(CC1CCN(Cc2ccccc2Cl)CC1)C(=O)c1cc2ccccc2[nH]1]

¹H NMR: (400 MHz, METHANOL-d₄) δ ppm 7.69-7.55 (m, 3H), 7.54-7.38 (m, 3H), 7.22 (br t, J=7.5 Hz, 1H), 7.07 (br t, J=7.5 Hz, 1H), 6.91-6.77 (m, 1H), 4.48 (br s, 2H), 3.77 (br s, 2H), 3.56 (br s, 4H), 3.15 (br t, J=12.1 Hz, 2H), 2.29-1.86 (m, 3H), 1.69-1.38 (m, 2H), 1.35-1.28 (m, 3H)

LCMS (ESI+): m/z 410.1 (M+H)

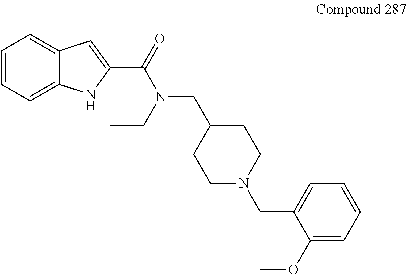 [SMILES: CCN(CC1CCN(Cc2ccccc2OC)CC1)C(=O)c1cc2ccccc2[nH]1]

¹H NMR: (400 MHz, METHANOL-d₄) δ ppm 7.66-7.58 (m, 1H), 7.52-7.35 (m, 3H), 7.21 (br t, J=7.7 Hz, 1H), 7.14-7.00 (m, 3H), 6.91-6.79 (m, 1H), 4.28 (br s, 2H), 3.90 (s, 3H), 3.75 (br s, 2H), 3.50 (br d, J=11.9 Hz, 4H), 3.01 (br t, J=12.1 Hz, 2H), 2.29-1.80 (m, 3H), 1.67-1.41 (m, 2H), 1.31 (br t, J=6.6 Hz, 3H)

LCMS (ESI+): m/z 406.2 (M+H)

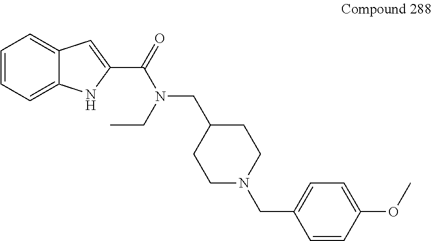 [SMILES: CCN(CC1CCN(Cc2ccc(OC)cc2)CC1)C(=O)c1cc2ccccc2[nH]1]

¹H NMR: (400 MHz, METHANOL-d₄) δ ppm 7.61 (br d, J=7.7 Hz, 1H), 7.46-7.35 (m, 3H), 7.25-7.17 (m, 1H), 7.11-6.96 (m, 3H), 6.91-6.79 (m, 1H), 4.20 (br s, 2H), 3.87-3.69 (m, 5H), 3.64-3.32 (m, 4H), 2.94 (br t, J=12.2 Hz, 2H), 2.12 (br s, 1H), 1.97 (br d, J=14.6 Hz, 2H), 1.67-1.37 (m, 1H), 1.37-1.36 (m, 1H), 1.30 (br s, 3H)

LCMS (ESI+): m/z 406.2 (M+H)

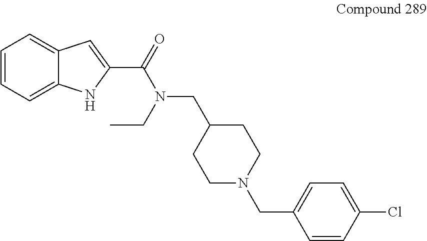 [SMILES: CCN(CC1CCN(Cc2ccc(Cl)cc2)CC1)C(=O)c1cc2ccccc2[nH]1]

¹H NMR: (400 MHz, METHANOL-d₄) δ ppm 7.61 (br d, J=7.5 Hz, 1H), 7.54-7.39 (m, 5H), 7.21 (br t, J=7.3 Hz, 1H), 7.07 (br t, J=7.5 Hz, 1H), 6.85 (br s, 1H), 4.28 (br s, 2H), 3.77 (br s, 2H), 3.50 (br d, J=11.9 Hz, 4H), 3.06-2.93 (m, 2H), 2.18-1.94 (m, 3H), 1.52 (br s, 2H), 1.32 (br d, J=6.6 Hz, 3H)

LCMS (ESI+): m/z 410.1 (M+H)

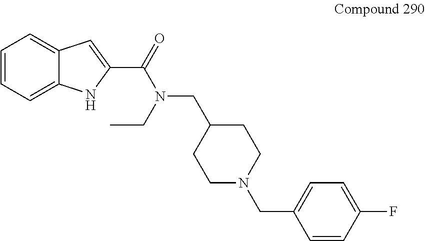 [SMILES: CCN(CC1CCN(Cc2ccc(F)cc2)CC1)C(=O)c1cc2ccccc2[nH]1]

¹H NMR: (400 MHz, METHANOL-d₄) δ ppm 7.62 (br d, J=7.5 Hz, 1H), 7.53 (br s, 2H), 7.43 (br d, J=7.9 Hz, 1H), 7.30-7.18 (m, 3H), 7.11-7.02 (m, 1H), 6.85 (br s, 1H), 4.28 (br s, 2H), 3.77 (br s, 2H), 3.50 (br d, J=11.0 Hz, 4H), 2.98 (br t, J=12.1 Hz, 2H), 2.24-1.89 (m, 3H), 1.66-1.36 (m, 2H), 1.31 (br d, J=6.2 Hz, 3H)

LCMS (ESI+): m/z 394.1 (M+H)

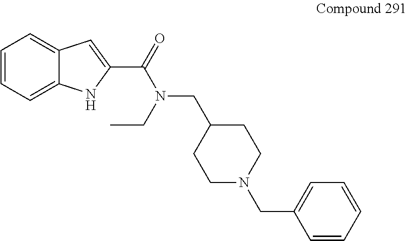 [SMILES: CCN(CC1CCN(Cc2ccccc2)CC1)C(=O)c1cc2ccccc2[nH]1]

¹H NMR: (400 MHz, METHANOL-d₄) δ ppm 7.61 (br d, J=7.9 Hz, 1H), 7.50-7.41 (m, 6H), 7.21 (br t, J=7.5 Hz, 1H), 7.10-7.03 (m, 1H), 6.91-6.76 (m, 1H), 4.28 (br s, 2H), 3.76 (br s, 2H), 3.50 (br d, J=11.5 Hz, 4H), 2.99 (br t, J=12.3 Hz, 2H), 2.30-1.88 (m, 3H), 1.67-1.39 (m, 2H), 1.31 (br t, J=6.6 Hz, 3H)

LCMS (ESI+): m/z 376.1 (M+H)

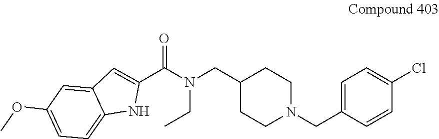 [SMILES: CCN(CC1CCN(Cc2ccc(Cl)cc2)CC1)C(=O)c1cc2cc(OC)ccc2[nH]1]

¹H NMR (400 MHz, METHANOL-d₄) δ ppm 7.47 (d, J=5.7 Hz, 4H) 7.29 (d, J=9.3 Hz, 1H) 7.06 (br. s., 1H) 6.86 (d, J=8.8 Hz, 1H) 6.81-6.74 (m, 1H) 4.26 (br. s., 2H) 3.83-3.67 (m, 6H) 3.48 (d, J=11.5 Hz, 3H) 3.34 (br. s., 2H) 2.96 (t, J=12.1 Hz, 2H) 2.12 (br. s., 1H) 1.97 (d, J=12.8 Hz, 2H) 1.30 (d, J=6.2 Hz, 3H)

LCMS (ESI+): m/z 440.1 (M+H)

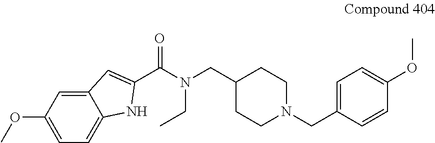 [SMILES: CCN(CC1CCN(Cc2ccc(OC)cc2)CC1)C(=O)c1cc2cc(OC)ccc2[nH]1]

¹H NMR (400 MHz, METHANOL-d₄) δ ppm 7.37 (d, J=7.9 Hz, 2H) 7.29 (d, J=8.8 Hz, 1H) 7.06 (d, J=2.2 Hz, 1H) 7.00 (d, J=8.4 Hz, 2H) 6.86 (dd, J=2.6, 8.8 Hz, 1H) 6.76 (br. s., 1H) 4.19 (s, 2H) 3.83-3.70 (m, 10H) 3.47 (d, J=11.9 Hz, 4H) 2.93 (t, J=11.9 Hz, 2H) 2.11 (br. s., 1H) 1.96 (d, J=13.7 Hz, 2H) 1.32-1.27 (m, 3H)

LCMS (ESI+): m/z 436.2 (M+H)

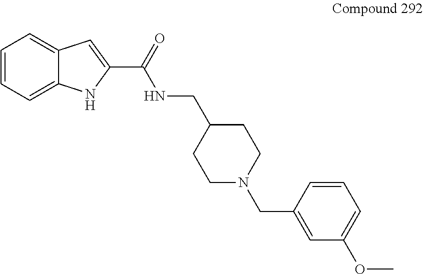 [SMILES: COc1cccc(CN2CCC(CNC(=O)c3cc4ccccc4[nH]3)CC2)c1]

¹H NMR: (400 MHz, METHANOL-d₄) δ ppm 7.59 (br d, J=7.28 Hz, 1H) 7.42 (br s, 2H) 7.22-7.24 (m, 1H) 7.05 (br s, 5H) 4.26 (br s, 2H) 3.83 (s, 3H) 3.52 (br d, J=11.47 Hz, 2H) 3.33-3.36 (m, 2H) 3.01 (br t, J=14.00 Hz, 2H) 2.05 (br d, J=16.32 Hz, 2H) 1.95 (br s, 1H) 1.45-1.58 (m, 2H)

LCMS (ESI+): m/z 378.2 (M+H)

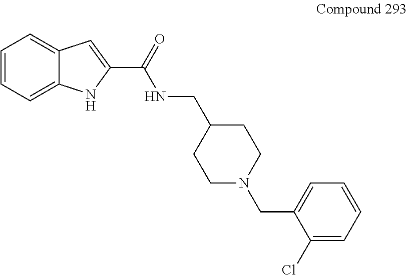 [SMILES: O=C(NCC1CCN(Cc2ccccc2Cl)CC1)c1cc2ccccc2[nH]1]

¹H NMR: (400 MHz, METHANOL-d₄) δ ppm 7.56-7.66 (m, 3H) 7.40-7.54 (m, 3H) 7.18-7.24 (m, 1H) 7.05 (br s, 1H) 7.03-7.09 (m, 1H) 4.49 (br s, 2H) 3.60 (br d, J=10.36 Hz, 2H) 3.34 (br d, J=4.63 Hz, 2H) 3.17 (br t, J=12.90 Hz, 1H) 3.12-3.22 (m, 1H) 3.12-3.22 (m, 1H) 1.93-2.10 (m, 3H) 1.49-1.62 (m, 2H)

LCMS (ESI+): m/z 382.1 (M+H)

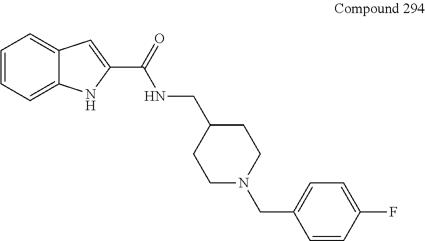 [SMILES: O=C(NCC1CCN(Cc2ccc(F)cc2)CC1)c1cc2ccccc2[nH]1]

¹H NMR: (400 MHz, METHANOL-d₄) δ ppm 7.51-7.61 (m, 3H) 7.43 (br d, J=8.38 Hz, 1H) 7.22 (q, J=7.86 Hz, 3H) 7.04 (br s, 2H) 4.29 (s, 2H) 3.52 (br d, J=10.80 Hz, 2H) 3.34 (br s, 2H) 3.00 (br t, J=13.01 Hz, 2H) 2.06 (br d, J=14.55 Hz, 2H) 1.96 (br s, 1H) 1.44-1.57 (m, 2H)

LCMS (ESI+): m/z 366.1 (M+H)

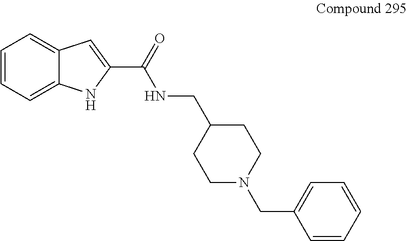 [SMILES: O=C(NCC1CCN(Cc2ccccc2)CC1)c1cc2ccccc2[nH]1]

¹H NMR: (400 MHz, METHANOL-d₄) δ ppm 7.59 (br d, J=7.06 Hz, 1H) 7.49 (br s, 5H) 7.43 (br d, J=8.16 Hz, 1H) 7.21 (br t, J=7.61 Hz, 1H) 7.02-7.08 (m, 1H) 7.02-7.08 (m, 1H) 4.29 (br s, 2H) 3.52 (br d, J=11.25 Hz, 2H) 3.34 (br s, 3H) 3.01 (br t, J=12.24 Hz, 2H) 2.05 (br d, J=13.45 Hz, 2H) 1.96 (br s, 1H) 1.45-1.59 (m, 2H)

LCMS (ESI+): m/z 348.1 (M+H)

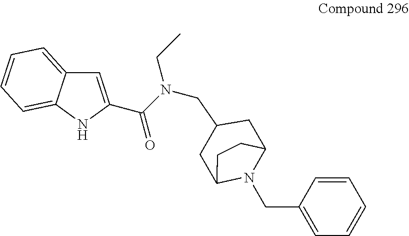 [SMILES: CCN(CC1CC2CCC(C1)N2Cc1ccccc1)C(=O)c1cc2ccccc2[nH]1]

¹H NMR (400 MHz, METHANOL-d₄) δ ppm 7.62 (br d, J=8.2 Hz, 1H), 7.50 (br s, 4H), 7.43 (br d, J=8.4 Hz, 1H), 7.22 (br t, J=8.0 Hz, 1H), 7.11-7.02 (m, 1H), 6.88 (br s, 1H), 4.19 (br s, 2H), 3.96-3.71 (m, 5H), 2.72-2.13 (m, 7H), 1.90 (br s, 2H), 1.74 (br s, 1H), 1.37-1.24 (m, 3H).

LCMS (ESI+): m/z 402.2 (M+H)

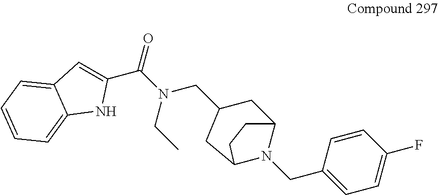 [SMILES: CCN(CC1CC2CCC(C1)N2Cc1ccc(F)cc1)C(=O)c1cc2ccccc2[nH]1]

¹H NMR (400 MHz, METHANOL-d₄) δ ppm 7.62 (d, J=8.3 Hz, 1H), 7.56 (br s, 2H), 7.43 (d, J=8.3 Hz, 1H), 7.28-7.19 (m, 3H), 7.10-7.04 (m, 1H), 6.88 (s, 1H), 4.18 (br s, 2H), 3.95-3.70 (m, 6H), 2.80-2.36 (m, 3H), 2.35-2.16 (m, 3H), 1.90 (br s, 2H), 1.36-1.23 (m, 4H). LCMS (ESI+): m/z 420.1 (M+H)

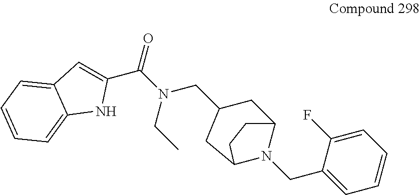 [SMILES: CCN(CC1CC2CCC(C1)N2Cc1ccccc1F)C(=O)c1cc2ccccc2[nH]1]

¹H NMR (400 MHz, METHANOL-d₄) δ ppm 7.62 (br d, J=7.9 Hz, 2H), 7.58-7.52 (m, 1H), 7.43 (d, J=8.3 Hz, 1H), 7.36-7.27 (m, 2H), 7.27-7.19 (m, 1H), 7.11-7.03 (m, 1H), 6.88 (s, 1H), 4.28 (br s, 2H), 4.04-3.74 (m, 6H), 2.71-2.38 (m, 3H), 2.26 (br d, J=12.3 Hz, 3H), 1.91 (br s, 2H), 1.36-1.23 (m, 4H) LCMS (ESI+): m/z 420.2 (M+H)

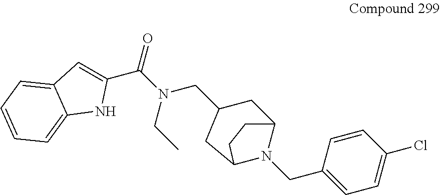 [SMILES: CCN(CC1CC2CCC(C1)N2Cc1ccc(Cl)cc1)C(=O)c1cc2ccccc2[nH]1]

¹H NMR (400 MHz, METHANOL-d₄) δ ppm 7.62 (d, J=8.3 Hz, 1H), 7.48-7.59 (m, 4H), 7.43 (d, J=7.9 Hz, 1H), 7.22 (t, J=7.7 Hz, 1H), 7.04-7.10 (m, 1H), 6.88 (s, 1H), 4.18 (br s, 2H), 3.71-4.00 (m, 6H), 2.42 (br s, 3H), 2.16-2.34 (m, 3H), 1.87 (br s, 2H), 1.23-1.35 (m, 4H) LCMS (ESI+): m/z 436.1 (M+H)

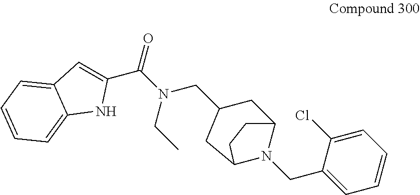 [SMILES: CCN(CC1CC2CCC(C1)N2Cc1ccccc1Cl)C(=O)c1cc2ccccc2[nH]1]

¹H NMR (400 MHz, METHANOL-d₄) δ ppm 7.68 (br s, 1H), 7.56-7.64 (m, 2H), 7.42-7.52 (m, 3H), 7.22 (t, J=7.7 Hz, 1H), 7.04-7.10 (m, 1H), 6.88 (s, 1H), 4.37 (br s, 2H), 3.75-4.05 (m, 6H), 2.51 (br s, 3H), 2.27-2.35 (m, 3H), 1.90 (br s, 2H), 1.23-1.36 (m, 4H)

LCMS (ESI+): m/z 436.1 (M+H)

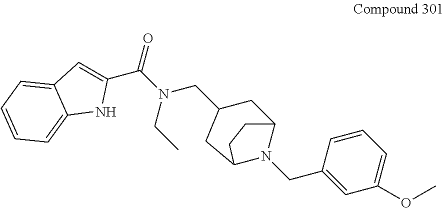 [SMILES: CCN(CC1CC2CCC(C1)N2Cc1cccc(OC)c1)C(=O)c1cc2ccccc2[nH]1]

¹H NMR (400 MHz, METHANOL-d₄) δ ppm 7.62 (d, J=8.3 Hz, 1H), 7.38-7.44 (m, 2H), 7.22 (t, J=7.7 Hz, 1H), 7.07 (br t, J=7.7 Hz, 4H), 6.88 (s, 1H), 4.15 (br s, 2H), 3.76-3.88 (m, 9H), 2.42-2.66 (m, 3H), 2.17-2.32 (m, 3H), 1.90 (br s, 2H), 1.23-1.37 (m, 4H).

LCMS (ESI+): m/z 432.2 (M+H)

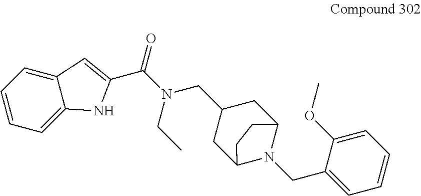 [SMILES: CCN(CC1CC2CCC(C1)N2Cc1ccccc1OC)C(=O)c1cc2ccccc2[nH]1]

¹H NMR (400 MHz, METHANOL-d₄) δ ppm 7.62 (d, J=7.9 Hz, 1H), 7.41-7.51 (m, 3H), 7.22 (t, J=7.7 Hz, 1H), 7.12 (d, J=8.3 Hz, 1H), 7.06 (q, J=7.7 Hz, 2H), 6.87 (s, 1H), 4.20 (br s, 2H), 3.84-4.01 (m, 7H), 3.76 (br s, 2H), 2.36-2.71 (m, 3H), 2.26 (brd, J=11.4 Hz, 3H), 1.88 (br s, 2H), 1.22-1.36 (m, 4H). LCMS (ESI+): m/z 432.2 (M+H)

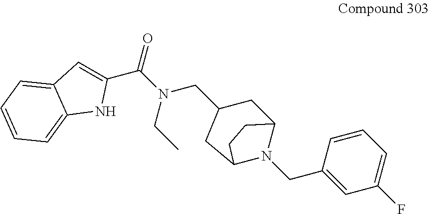 [SMILES: CCN(CC1CC2CCC(C1)N2Cc1cccc(F)c1)C(=O)c1cc2ccccc2[nH]1]

¹H NMR (400 MHz, METHANOL-d₄) δ ppm 7.62 (d, J=7.9 Hz, 1H), 7.52 (br d, J=7.0 Hz, 1H), 7.43 (d, J=8.3 Hz, 1H), 7.35 (br s, 2H), 7.20-7.27 (m, 2H), 7.03-7.10 (m, 1H), 6.88 (s, 1H), 4.21 (br s, 2H), 3.73-3.96 (m, 7H), 2.19-2.41 (m, 4H), 1.91 (br s, 2H), 1.25-1.36 (m, 5H).

LCMS (ESI+): m/z 420.1 (M+H)

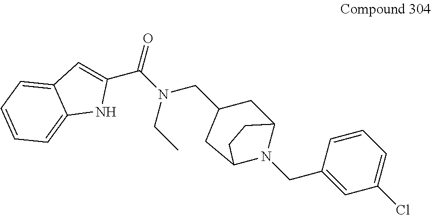 [SMILES: CCN(CC1CC2CCC(C1)N2Cc1cccc(Cl)c1)C(=O)c1cc2ccccc2[nH]1]

¹H NMR (400 MHz, METHANOL-d₄) δ ppm 7.62 (br d, J=7.5 Hz, 2H), 7.42-7.51 (m, 4H), 7.22 (t, J=7.7 Hz, 1H), 7.04-7.10 (m, 1H), 6.88 (s, 1H), 4.19 (br s, 2H), 3.74-3.91 (m, 7H), 2.38-2.58 (m, 2H), 2.17-2.33 (m, 3H), 1.87 (s, 1H), 1.29 (t, J=7.2 Hz, 5H).

LCMS (ESI+): m/z 436.1 (M+H)

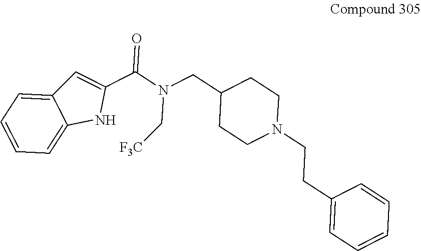 [SMILES: O=C(c1cc2ccccc2[nH]1)N(CC1CCN(CCc2ccccc2)CC1)CC(F)(F)F]

¹H NMR (400 MHz, METHANOL-d₄) δ ppm 7.61 (d, J=7.9 Hz, 1H) 7.42 (d, J=8.2 Hz, 1H) 7.25-7.18 (m, 3H) 7.16-7.10 (m, 3H), 7.08-7.02 (m, 1H) 6.86 (s, 1H) 4.42 (br d, J=8.4 Hz, 2H) 3.77 (br s, 2H) 2.94 (br d, J=10.8 Hz, 2H) 2.76-2.67 (m, 2H) 2.55-2.45 (m, 2H) 1.99 (br t, J=11.2 Hz, 2H) 1.82 (br s, 1H) 1.66 (br d, J=12.6 Hz, 2H) 1.28-1.14 (m, 2H)

LCMS (ESI+): m/z 444.3 (M+H)

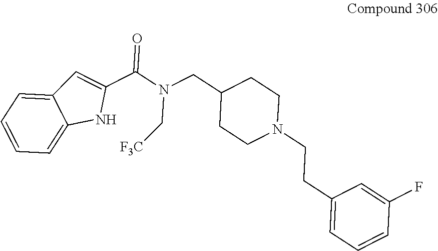 [SMILES: O=C(c1cc2ccccc2[nH]1)N(CC1CCN(CCc2cccc(F)c2)CC1)CC(F)(F)F]

¹H NMR (400 MHz, METHANOL-d₄) δ ppm 7.61 (d, J=7.7 Hz, 1H) 7.42 (d, J=8.4 Hz, 1H) 7.26-7.18 (m, 2H) 7.09-7.03 (m, 1H) 6.98-6.83 (m, 4H) 4.43 (br d, J=8.4 Hz, 2H) 3.77 (br s, 2H) 2.95 (br d, J=9.9 Hz, 2H) 2.77-2.69 (m, 2H) 2.56-2.47 (m, 2H) 2.02 (br t, J=11.5 Hz, 2H) 1.83 (br s, 1H) 1.67 (br d, J=12.1 Hz, 2H) 1.24 (br d, J=16.8 Hz, 1H) 1.31-1.15 (m, 1H)

LCMS (ESI+): m/z 462.3 (M+H)

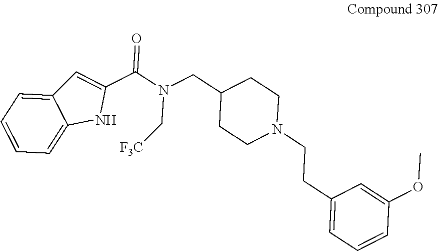 [SMILES: COc1cccc(CCN2CCC(CN(CC(F)(F)F)C(=O)c3cc4ccccc4[nH]3)CC2)c1]

¹H NMR (400 MHz, METHANOL-d₄) δ ppm 7.61 (d, J=7.9 Hz, 1H) 7.42 (d, J=8.2 Hz, 1H) 7.21 (t, J=7.6 Hz, 1H) 7.13 (br t, J=8.2 Hz, 1H) 7.08-7.02 (m, 1H) 6.86 (s, 1H) 6.73-6.68 (m, 1H) 6.70 (br s, 2H) 4.42 (br d, J=8.2 Hz, 2H) 3.72 (s, 5H) 2.94 (br d, J=10.4 Hz, 2H) 2.73-2.64 (m, 2H) 2.55-2.45 (m, 2H) 2.05-1.95 (m, 2H) 1.82 (br s, 1H) 1.66 (br d, J=12.3 Hz, 2H) 1.24 (br d, J=17.2 Hz, 2H)

LCMS (ESI+): m/z 474.3 (M+H)

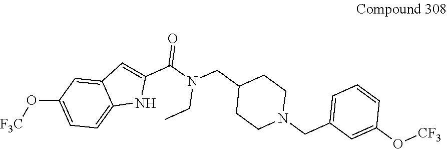 [SMILES: CCN(CC1CCN(Cc2cccc(OC(F)(F)F)c2)CC1)C(=O)c1cc2cc(OC(F)(F)F)ccc2[nH]1]

¹H NMR (400 MHz, METHANOL-d₄) δ ppm 7.62-7.55 (m, 1H) 7.53-7.46 (m, 4H) 7.42 (br d, J=8.2 Hz, 1H) 7.19-7.10 (m, 1H) 6.88 (br s, 1H) 4.34 (br s, 1H) 4.40-4.29 (m, 1H) 3.79-3.47 (m, 6H) 3.01 (br t, J=11.7 Hz, 2H) 2.15 (br s, 1H) 2.04-1.94 (m, 2H) 1.56 (br s, 2H) 1.30 (br t, J=6.9 Hz, 3H)

LCMS (ESI+): m/z 544.1 (M+H)

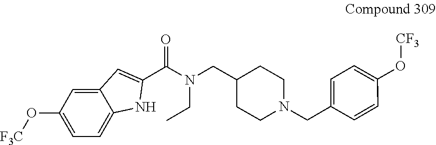 [SMILES: CCN(CC1CCN(Cc2ccc(OC(F)(F)F)cc2)CC1)C(=O)c1cc2cc(OC(F)(F)F)ccc2[nH]1]

¹H NMR (400 MHz, METHANOL-d₄) δ ppm 7.61 (br d, J=7.5 Hz, 2H) 7.54-7.46 (m, 2H) 7.39 (br d, J=8.2 Hz, 2H) 7.19-7.10 (m, 1H) 6.89 (br s, 1H) 4.32 (br s, 2H) 3.80-3.48 (m, 6H) 3.00 (br t, J=12.1 Hz, 2H) 2.20-2.08 (m, 1H) 2.03-1.94 (m, 2H) 1.62-1.39 (m, 2H) 1.33-1.27 (m, 3H)

LCMS (ESI+): m/z 544.1 (M+H)

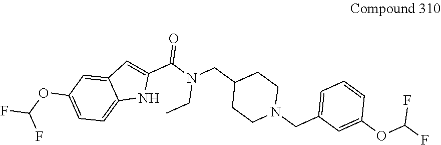 [SMILES: CCN(CC1CCN(Cc2cccc(OC(F)F)c2)CC1)C(=O)c1cc2cc(OC(F)F)ccc2[nH]1]

¹H NMR (400 MHz, METHANOL-d₄) δ ppm 7.52 (t, J=7.89 Hz, 1H) 7.24-7.48 (m, 5H) 6.99-7.12 (m, 1H) 6.80-6.93 (m, 2H) 6.51-6.73 (m, 1H) 4.21-4.40 (m, 2H) 3.75 (br s, 2H) 3.43-3.63 (m, 1H) 3.51 (br d, J=11.69 Hz, 2H) 3.00 (br t, J=12.13 Hz, 2H) 2.07-2.22 (m, 1H) 1.90-2.06 (m, 2H) 1.42-1.63 (m, 2H) 1.23-1.36 (m, 4H)

LCMS (ESI+): m/z 508.1 (M+H)

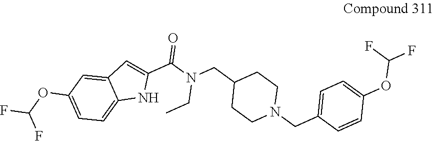 [SMILES: CCN(CC1CCN(Cc2ccc(OC(F)F)cc2)CC1)C(=O)c1cc2cc(OC(F)F)ccc2[nH]1]

¹H NMR (400 MHz, METHANOL-d₄) δ ppm 7.35-7.45 (m, 4H) 7.01-7.13 (m, 3H) 6.76-7.00 (m, 2H) 6.50-6.73 (m, 1H) 3.47-3.78 (m, 7H) 3.00 (br s, 2H) 2.18 (br s, 2H) 1.75 (br s, 2H) 1.35-1.51 (m, 2H) 1.28 (br t, J=6.80 Hz, 4H)

LCMS (ESI+): m/z 508.3 (M+H)

### Example 16: General Protocol M for Synthesis of Exemplary Compounds

General Protocol M to synthesize exemplary compounds of Formula (I) is described in Scheme 13 and the procedures set forth below.

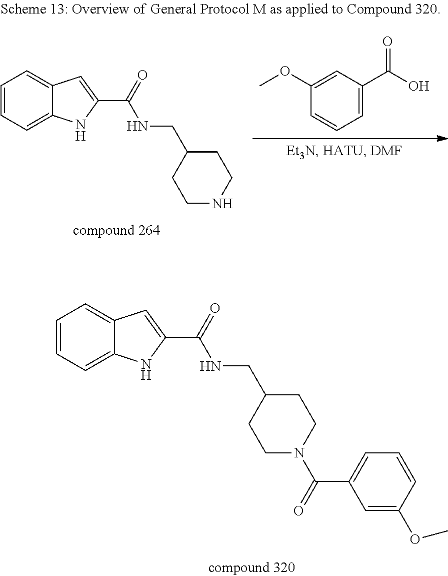

Procedure for the preparation of compound 320: To a mixture of compound 264 (40.0 mg, 136.2 μmol, 1.0 eq, HCl) and 3-methoxy-benzoic acid (16.6 mg, 108.9 μmol, 0.8 eq) in 3 mL of DMF was added HATU (62.1 mg, 163.4 μmol, 1.2 eq), Et₃N (41.3 mg, 408.5 μmol, 3.0 eq) in one portion at 20° C. under N₂. The mixture was then stirred for 16 hours at 20° C. The reaction mixture was diluted with 5 mL of water and extracted with three 5 mL potions of DCM. The combined organic layers were washed twice with 5 mL potions of brine, dried over Na₂SO₄, filtered and concentrated under reduced pressure to give a residue. The residue was purified by prep-HPLC (TFA condition) to afford 4.0 mg (7%) of compound 320 as yellow solid.

¹H NMR: (400 MHz, METHANOL-d₄) δ ppm 7.57 (d, J=7.94 Hz, 1H) 7.41 (dd, J=8.38, 0.88 Hz, 1H) 7.31-7.36 (m, 1H) 7.18 (ddd, J=8.32, 7.11, 1.10 Hz, 1H) 6.98-7.05 (m, 3H) 6.90-6.94 (m, 1H) 6.90-6.94 (m, 1H) 3.79 (s, 3H) 3.72 (br s, 2H) 3.01-3.15 (m, 2H) 2.72-2.98 (m, 2H) 1.86-2.00 (m, 2H) 1.74 (br d, J=14.33 Hz, 1H) 1.20-1.34 (m, 2H)

LCMS (ESI+): m/z 392.1 (M+H)

The following compounds were prepared according to General Protocol M:

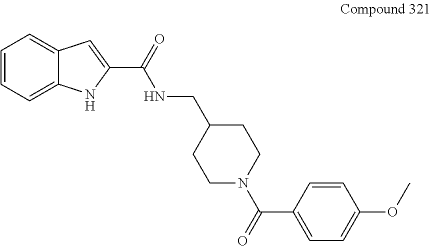 [SMILES: COc1ccc(C(=O)N2CCC(CNC(=O)c3cc4ccccc4[nH]3)CC2)cc1]

¹H NMR: (400 MHz, METHANOL-d₄) δ ppm 7.56-7.59 (m, 1H) 7.41 (dd, J=8.38, 0.88 Hz, 1H) 7.33-7.37 (m, 2H) 7.19 (ddd, J=8.32, 7.11, 1.10 Hz, 1H) 7.02-7.05 (m, 2H) 6.95-6.98 (m, 2H) 4.48-4.64 (m, 2H) 3.81 (s, 1H) 3.80-3.82 (m, 1H) 3.80-3.82 (m, 1H) 2.73-3.21 (m, 4H) 1.83-2.00 (m, 2H) 1.71-1.81 (m, 1H) 1.27 (br s, 2H)

LCMS (ESI+): m/z 392.1 (M+H)

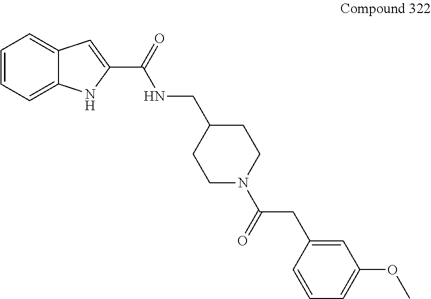 [SMILES: COc1cccc(CC(=O)N2CCC(CNC(=O)c3cc4ccccc4[nH]3)CC2)c1]

¹H NMR: (400 MHz, METHANOL-d₄) δ ppm 7.57 (d, J=8.16 Hz, 1H) 7.41 (dd, J=8.27, 0.77 Hz, 1H) 7.14-7.21 (m, 2H) 7.00-7.06 (m, 2H) 6.78-6.81 (m, 2H) 6.75 (dd, J=8.16, 2.43 Hz, 1H) 6.72-6.76 (m, 1H) 6.72-6.76 (m, 1H) 6.72-6.76 (m, 1H) 4.55 (br d, J=13.45 Hz, 1H) 3.99 (br d, J=13.89 Hz, 1H) 3.70-3.74 (m, 5H) 3.21 (d, J=5.95 Hz, 2H) 2.97-3.06 (m, 1H) 2.64 (td, J=12.84, 2.76 Hz, 1H) 1.75-1.90 (m, 2H) 1.67 (br d, J=12.79 Hz, 1H) 1.12 (qd, J=12.27, 3.97 Hz, 1H) 0.92 (qd, J=12.35, 3.97 Hz, 1H) 0.86-0.97 (m, 1H)

LCMS (ESI+): m/z 406.2 (M+H)

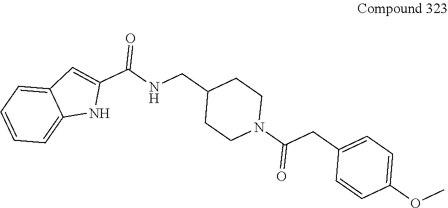 [SMILES: COc1ccc(CC(=O)N2CCC(CNC(=O)c3cc4ccccc4[nH]3)CC2)cc1]

¹H NMR: (400 MHz, METHANOL-d₄) δ ppm 7.57 (d, J=7.94 Hz, 1H) 7.41 (dd, J=8.16, 0.88 Hz, 1H) 7.12-7.21 (m, 3H) 7.00-7.05 (m, 2H) 6.82 (d, J=8.82 Hz, 2H) 4.54 (br d, J=12.57 Hz, 1H) 4.00 (br d, J=14.33 Hz, 1H) 3.66-3.72 (m, 5H) 3.22 (dd, J=6.84, 2.43 Hz, 2H) 3.01 (br t, J=11.58 Hz, 1H) 2.57-2.67 (m, 1H) 1.75-1.91 (m, 3H) 1.67 (br d, J=12.57 Hz, 1H) 1.06-1.17 (m, 1H) 0.86-0.98 (m, 1H)

LCMS (ESI+): m/z 406.1 (M+H)

### Example 17: General Protocol N for Synthesis of Exemplary Compounds

General Protocol N to synthesize exemplary compounds of Formula (I) is described in Scheme 14 and the procedures set forth below.

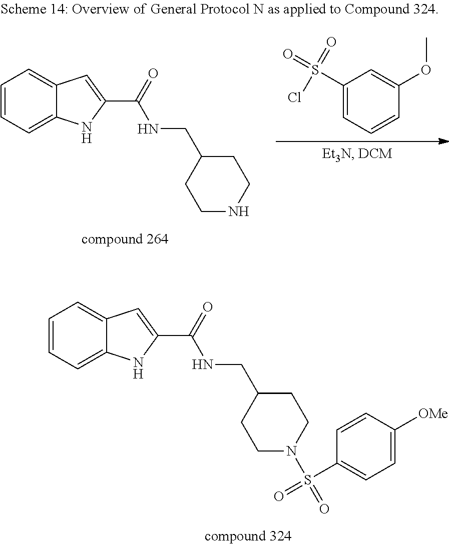

Procedure for the preparation of compound 324: To a mixture of compound 264 (40.0 mg, 136.2 μmol, 1.0 eq, HCl) and 3-methoxyphenyl sulfonyl chloride (28.1 mg, 136.2 μmol, 1.0 eq) in 3 mL of DCM was added Et₃N (41.3 mg, 408.5 μmol, 3.0 eq) in one portion at 20 C under N₂. The mixture was stirred at 20° C. for 16 hours. The reaction mixture was diluted with 5 mL of water and extracted with three 5 mL potions of DCM. The combined organic layers were washed twice with 5 mL potions of brine, dried over Na₂SO₄, filtered and concentrated under reduced pressure to give a residue. The residue was purified by prep-HPLC (TFA condition) to afford 4.0 mg of compound 324 (6% yield) as white solid.

¹H NMR: (400 MHz, METHANOL-d₄) δ ppm 7.56 (d, J=8.16 Hz, 1H) 7.45-7.50 (m, 1H) 7.38-7.42 (m, 1H) 7.30 (dd, J=8.05, 1.21 Hz, 1H) 7.21-7.24 (m, 1H) 7.14-7.19 (m, 2H) 6.99-7.05 (m, 2H) 3.83 (s, 3H) 3.76 (br d, J=11.69 Hz, 2H) 3.23 (d, J=7.06 Hz, 2H) 2.28-2.36 (m, 2H) 1.81 (br d, J=12.79 Hz, 2H) 1.55-1.62 (m, 1H) 1.27-1.36 (m, 2H)

LCMS (ESI+): m/z 428.1 (M+H)

The following compounds were prepared according to General Protocol N:

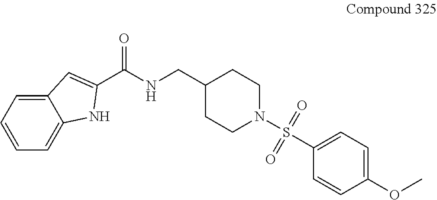 [SMILES: COc1ccc(S(=O)(=O)N2CCC(CNC(=O)c3cc4ccccc4[nH]3)CC2)cc1]

¹H NMR: (400 MHz, METHANOL-d₄) δ ppm 9.12 (br s, 1H) 7.69 (ddd, J=8.38, 1.43, 0.77 Hz, 2H) 7.64 (br d, J=7.50 Hz, 1H) 7.42 (br d, J=8.16 Hz, 1H) 7.29-7.32 (m, 1H) 7.11-7.18 (m, 1H) 6.96-7.01 (m, 2H) 6.80 (br s, 1H) 6.23 (br s, 1H) 3.86 (dd, J=1.32, 0.66 Hz, 3H) 3.79 (br d, J=10.36 Hz, 2H) 3.35 (br s, 2H) 2.26 (t, J=11.69 Hz, 2H) 1.81 (br d, J=13.23 Hz, 2H) 1.62 (br s, 1H) 1.38-1.46 (m, 2H)

LCMS (ESI+): m/z 428.0 (M+H)

### Example 18: General Protocol O for Synthesis of Exemplary Compounds

General Protocol O to synthesize exemplary compounds of Formula (I) is described in Scheme 15 and the procedures set forth below.

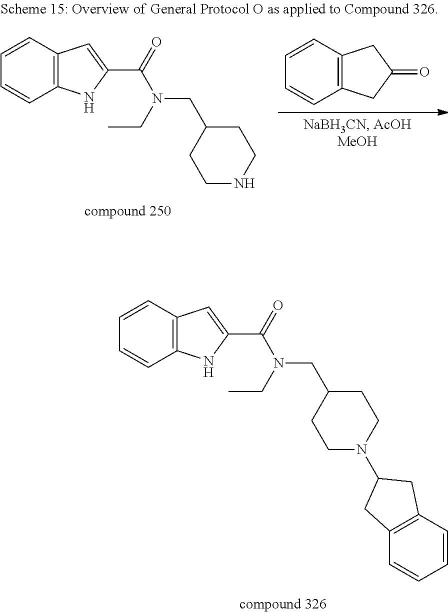

To the mixture of compound 250 (40.0 mg, 140.2 μmol, 1.0 eq), 2-indanone (55.6 mg, 420.5 μmol, 3.0 eq) and AcOH (8.4 mg, 140.2 μmol, 1.0 eq) in 1 mL of MeOH was added NaBH₃CN (17.6 mg, 280.3 μmol, 2.0 eq) in batches at 20° C. The reaction mixture was stirred at 80° C. for 16 hrs. The reaction mixture was quenched by addition of 3 mL of water at 20° C., and then diluted with 5 mL of water and extracted with three 5 mL potions of DCM. The combined organic layers were washed twice with brine, dried over Na₂SO₄, filtered and concentrated under reduced pressure to give a residue. The residue was purified by prep-HPLC (TFA condition) to afford 7.6 mg of compound 326 (10% yield) as white solid (TFA salt).

¹H NMR: (400 MHz, METHANOL-d₄) δ ppm 7.61 (d, J=7.94 Hz, 1H) 7.43 (d, J=8.38 Hz, 1H) 7.20-7.29 (m, 5H) 7.04-7.09 (m, 1H) 6.85-6.90 (m, 1H) 4.06 (br t, J=8.05 Hz, 1H) 3.79 (br s, 2H) 3.63 (br d, J=11.69 Hz, 3H) 3.38-3.48 (m, 2H) 3.12-3.23 (m, 3H) 3.05 (br t, J=12.24 Hz, 1H) 2.99-3.09 (m, 1H) 2.18 (br s, 1H) 2.06 (br d, J=13.89 Hz, 2H) 1.53 (br s, 2H) 1.33 (br t, J=6.95 Hz, 3H)

LCMS (ESI+): m/z 402.1 (M+H)

The following compounds were prepared according to General Protocol O:

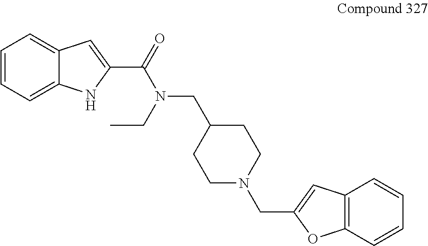 [SMILES: CCN(CC1CCN(Cc2cc3ccccc3o2)CC1)C(=O)c1cc2ccccc2[nH]1]

¹H NMR: (400 MHz, METHANOL-d₄) δ ppm 7.67 (d, J=7.72 Hz, 1H) 7.61 (br d, J=8.16 Hz, 1H) 7.55 (d, J=8.38 Hz, 1H) 7.41 (dd, J=7.72, 5.73 Hz, 2H) 7.27-7.33 (m, 1H) 7.21 (t, J=7.28 Hz, 1H) 7.12 (s, 1H) 7.03-7.09 (m, 1H) 6.84 (br s, 1H) 4.55 (s, 2H) 3.77 (br s, 2H) 3.61 (br d, J=12.57 Hz, 4H) 3.09 (br t, J=12.35 Hz, 2H) 2.15 (br s, 1H) 2.02 (br d, J=13.89 Hz, 2H) 1.54 (br s, 2H) 1.31 (br t, J=6.84 Hz, 3H)

LCMS (ESI+): m/z 416.1 (M+H)

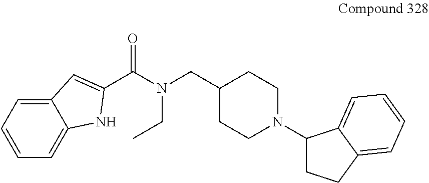 [SMILES: CCN(CC1CCN(C2CCc3ccccc32)CC1)C(=O)c1cc2ccccc2[nH]1]

¹H NMR: (400 MHz, METHANOL-d₄) δ ppm 7.59 (d, J=7.94 Hz, 1H) 7.54 (br d, J=7.94 Hz, 1H) 7.38-7.43 (m, 3H) 7.31-7.36 (m, 1H) 7.16-7.22 (m, 1H) 7.02-7.07 (m, 1H) 6.82 (s, 1H) 3.74 (br s, 2H) 3.44-3.58 (m, 3H) 3.22-3.27 (m, 1H) 2.85-3.19 (m, 5H) 2.42-2.54 (m, 2H) 2.09 (br s, 1H) 1.96 (br s, 2H) 1.46 (br s, 2H) 1.29 (t, J=7.06 Hz, 3H)

LCMS (ESI+): m/z 402.1 (M+H)

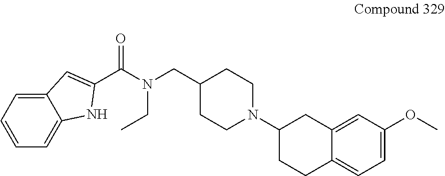 [SMILES: CCN(CC1CCN(C2CCc3ccc(OC)cc3C2)CC1)C(=O)c1cc2ccccc2[nH]1]

¹H NMR: (400 MHz, METHANOL-d₄) δ ppm 7.59 (d, J=8.16 Hz, 1H) 7.41 (d, J=8.38 Hz, 1H) 7.19 (t, J=7.61 Hz, 1H) 7.01-7.07 (m, 1H) 6.93 (br d, J=7.94 Hz, 1H) 6.81 (s, 1H) 6.58-6.67 (m, 2H) 3.71 (s, 4H) 3.46-3.64 (m, 2H) 3.11 (s, 4H) 2.80 (br s, 5H) 2.44 (s, 2H) 1.77-2.30 (m, 5H) 1.59 (s, 2H) 1.29 (br t, J=6.95 Hz, 4H)

LCMS (ESI+): m/z 446.2 (M+H)

### Example 19: General Protocol P for Synthesis of Exemplary Compounds

General Protocol P to synthesize exemplary compounds of Formula (I) is described in Scheme 16 and the procedures set forth below.

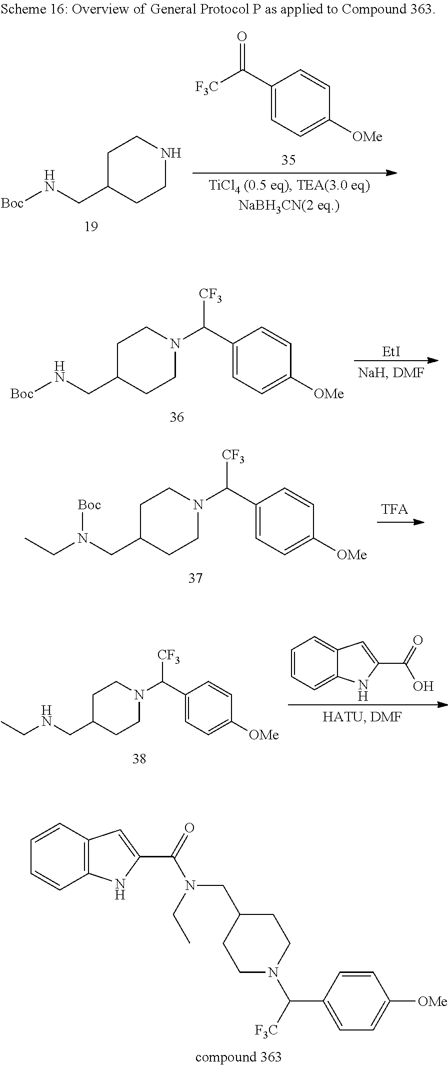

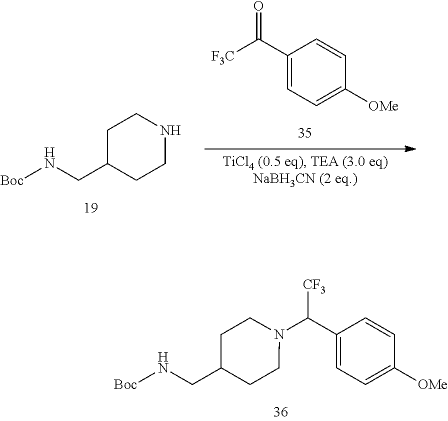 [SMILES: COc1ccc(C(=O)C(F)(F)F)cc1 ; CC(C)(C)OC(=O)NCC1CCNCC1 ; "TiCl4 (0.5 eq), TEA (3.0 eq)NaBH3CN (2 eq.)" ; COc1ccc(C(N2CCC(CNC(=O)OC(C)(C)C)CC2)C(F)(F)F)cc1]

Procedure for the preparation of compound 36: To a solution of compound 19 (100.0 mg, 467 μmol, 1.0 eq), trifluoroketone 35 (95.3 mg, 467 μmol, 1.0 eq) in 2 mL of DCM was added TiCl₄ (44.3 mg, 233 μmol, 0.5 eq), followed by dropwise addition of TEA (142 mg, 1.4 μmol, 3.0 eq). The mixture was stirred at 25° C. for 12 hrs, then NaBH₃CN (58.6 mg, 933 μmol, 2.0 eq) in 1 mL of MeOH was added. The mixture was stirred at 25° C. for another 2 hrs. LCMS showed the reaction was complete. The reaction mixture was quenched by adding 5 mL of 1N HCl at 0° C., and then extracted with three 3 mL portions of DCM. The combined organic layers were dried over Na₂SO₄, filtered and concentrated under reduced pressure to give a residue. The crude product was purified by TLC (SiO₂, eluting with petroleum ether/ethyl acetate=3/1) to give 80.0 mg (43%) of compound 36 as a yellow oil.

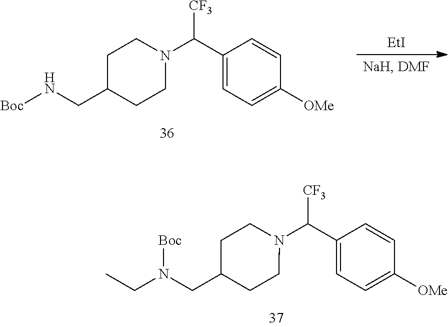

Procedure for the preparation of compound 37: To a solution of compound 36 (80.0 mg, 198.8 μmol, 1.0 eq) in 2 mL of DMF was added NaH (17.9 mg, 298.2 μmol, 60% purity, 1.5 eq) at 0° C. and the mixture was stirred for 10 min. EtI (62.0 mg, 398 μmol, 2.0 eq) was added at that temperature. The mixture was stirred at 25° C. for 3 hrs. The reaction was monitored by TLC and allowed to run until complete. The reaction mixture was quenched by adding 5 mL of saturated aqueous NH₄Cl at 0° C., and then extracted with three 3 mL portions of ethyl acetate. The combined organic layers were dried over Na₂SO₄, filtered and concentrated under reduced pressure to give 85 mg the crude product compound 37 as yellow oil.

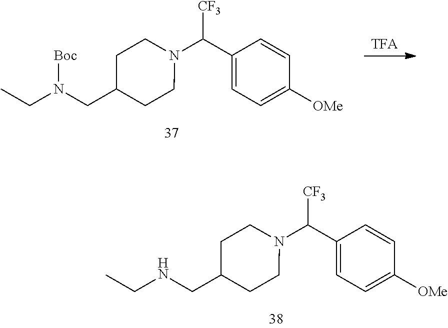 [SMILES: CCN(CC1CCN(C(c2ccc(OC)cc2)C(F)(F)F)CC1)C(=O)OC(C)(C)C ; CCNCC1CCN(C(c2ccc(OC)cc2)C(F)(F)F)CC1 ; "TFA"]

Procedure for the preparation of compound 38: To a solution of compound 37 (85.0 mg, 197 μmol, 1.00 eq) in 1.5 mL of DCM was added TFA (225 mg, 2.0 μmol, 10.0 eq). The mixture was stirred at 25° C. for 1 hour. LCMS showed the reaction was complete. The mixture was concentrated to give 60 mg of the crude product 38 as yellow oil, which was used in the next step without further purification.

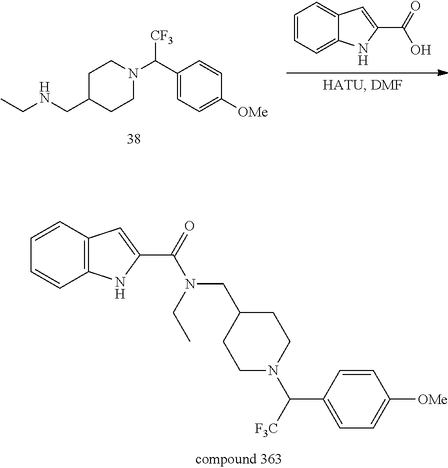

Procedure for the preparation of compound 363: To a solution of compound 38 (65.0 mg, 0.2 μmol, 1.00 eq) and 1H-indole-2-carboxylic acid (31.7 mg, 0.2 μmol, 1.00 eq) in 2 mL of DMF was added HATU (74.8 mg, 0.2 μmol, 1.00 eq) and TEA (39.8 mg, 0.4 μmol, 2.00 eq). The mixture was stirred at 25° C. for 12 hours. The reaction was complete as monitored by LCMS.

The reaction mixture was quenched by adding 5 mL of saturated aqueous NH₄Cl, and then extracted with three 3 mL portions of ethyl acetate. The combined organic layers were concentrated under reduced pressure to give a residue. The crude product was purified by HPLC to give 1.4 mg of the TFA salt of compound 363 as a yellow solid.

¹H NMR (400 MHz, METHANOL-d4) δ ppm 7.59 (d, J=7.72 Hz, 1H) 7.40 (d, J=8.60 Hz, 1H) 7.29 (br s, 2H) 7.18 (t, J=7.17 Hz, 1H) 7.04 (t, J=7.39 Hz, 1H) 6.92 (br d, J=8.16 Hz, 2H) 6.77 (br s, 1H) 4.14-4.42 (m, 1H) 3.78 (s, 3H) 3.36-3.75 (m, 4H) 3.03 (br s, 2H) 2.41 (br s, 1H) 2.17 (br t, J=7.39 Hz, 2H) 2.01 (br d, J=5.95 Hz, 1H) 1.54-1.82 (m, 4H) 1.24-1.27 (m, 4H)

LCMS (ESI+): m/z 474.3 (M+H)

The following compounds were prepared analogously:

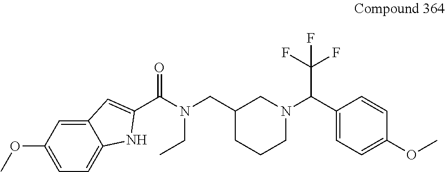

¹H NMR (400 MHz, CHLOROFORM-d) δ ppm 9.23-9.38 (m, 1H) 7.32 (d, J=8.77 Hz, 1H) 7.23 (br s, 2H) 7.06 (s, 1H) 6.96 (dd, J=8.77, 2.63 Hz, 1H) 6.84 (br d, J=8.33 Hz, 2H) 6.70 (br d, J=10.52 Hz, 1H) 3.97-4.09 (m, 1H) 3.86 (s, 3H) 3.78 (d, J=3.95 Hz, 3H) 3.61-3.75 (m, 1H) 3.51 (br s, 1H) 2.70-2.92 (m, 2H) 2.36-2.52 (m, 1H) 1.92-2.34 (m, 3H) 1.67 (br d, J=10.09 Hz, 2H) 1.47-1.61 (m, 1H) 1.24-1.40 (m, 3H) 1.05 (br s, 1H)

LCMS (ESI+): m/z 504.2 (M+H)

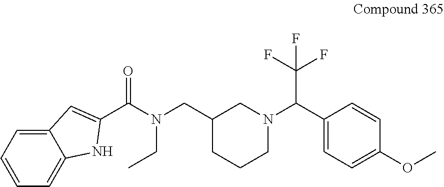 [SMILES: CCN(CC1CCCN(C(c2ccc(OC)cc2)C(F)(F)F)C1)C(=O)c1cc2ccccc2[nH]1]

¹H NMR (400 MHz, DMSO-d₆) δ ppm 11.48 (br s, 1H) 7.57 (br d, J=7.94 Hz, 1H) 7.40 (d, J=8.16 Hz, 1H) 7.11-7.28 (m, 3H) 6.97-7.04 (m, 1H) 6.85 (br s, 2H) 6.67 (br s, 1H) 4.44 (br s, 1H) 3.70 (s, 3H) 3.55 (br s, 3H) 2.67-2.88 (m, 2H) 1.94 (br s, 2H) 1.61-1.63 (m, 1H) 1.55 (br s, 2H) 1.39 (br s, 1H) 1.10-1.21 (m, 3H) 0.82 (br s, 1H)

LCMS (ESI+): m/z 474.2 (M+H)

### Example 20: General Protocol Q for Synthesis of Exemplary Compounds

General Protocol Q to synthesize exemplary compounds of Formula (I) is described in Scheme 17 and the procedures set forth below.

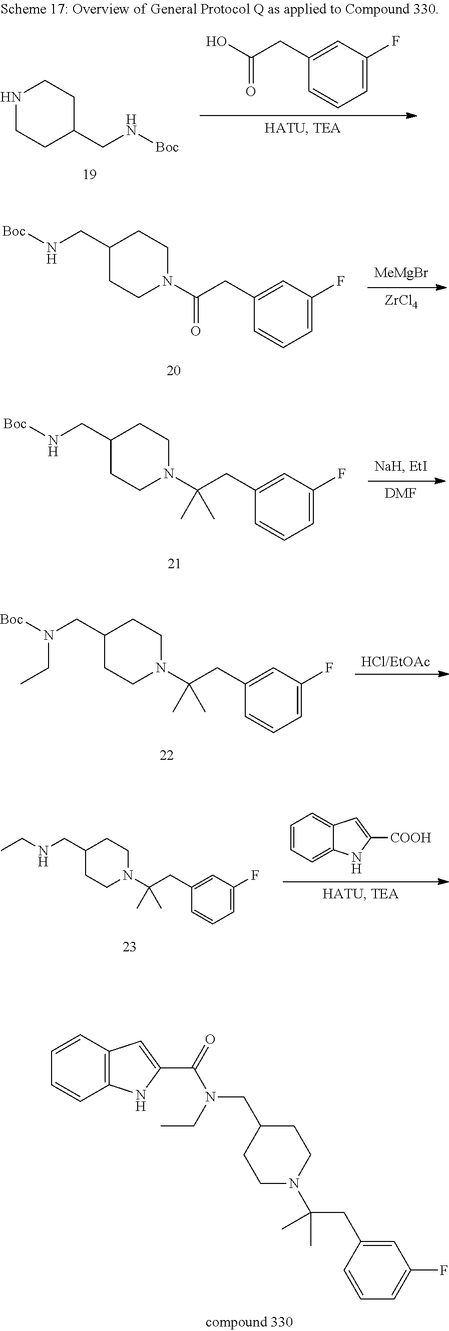

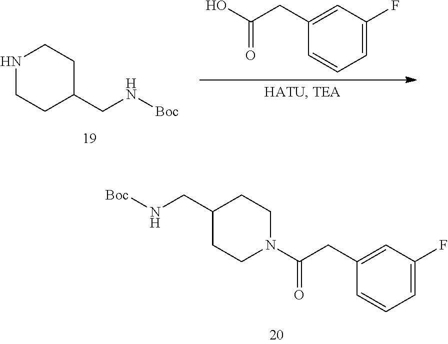

Procedure for the preparation of compound 20: A mixture of piperidine 19 (10.0 g, 46.7 mmol, 1.0 eq), 2-(3-fluorophenyl)acetic acid (7.2 g, 46.7 mmol, 1.0 eq), HATU (17.7 g, 46.7 mmol, 1.0 eq), and TEA (9.4 g, 93.3 mmol, 2.0 eq) in 100 mL of DMF was stirred at 25° C. for 1 hour. The reaction was monitored by TLC and allowed to run until completion. The reaction mixture was diluted with 300 mL of ethyl acetate and washed twice with 300 mL of water. The combined organic layers were washed five times with 200 mL of brine, dried over Na₂SO₄, filtered and concentrated under reduced pressure to give an oil which was purified by flash column chromatography (SiO₂, eluting with petroleum ether/ethyl acetate=1/1 to 1/1) to give 41.0 g of compound 20 as a yellow oil.

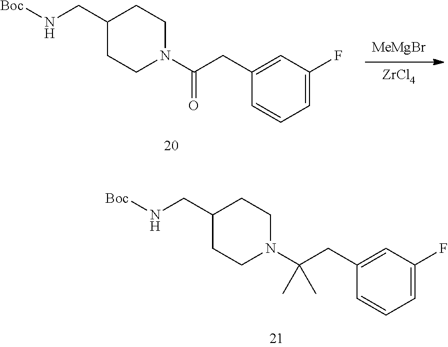

Procedure for the preparation of compound 21: A mixture of compound 20 (41 g, 39.1 mmol, 1.0 eq) in 140 mL of THF was cooled to −40° C., then ZrCl₄ (10.0 g, 43.0 mmol, 1.1 eq) was added and stirred at −40° C. for 0.5 hour, then MeMgBr (3M, 78 mL, 6.0 eq) was added slowly and the temperature kept at −20° C. The mixture was stirred cooled in an ice-bath for 15 min, then stirred at 25° C. for 1 hour under N₂ atmosphere. The reaction was monitored by TLC and allowed to run until completion. The reaction mixture was quenched by adding 3.5 L of icy saturated aqueous NH₄Cl, then added HCl (1M in water, ˜600 mL) until the reaction liquid become a little clearer. The mixture was extracted with three 500 mL portions of ethyl acetate. The combined organic layers were washed with 1000 mL of brine, dried over Na₂SO₄, filtered and concentrated under reduced pressure to give a solid. The residue was washed by the mixture of petroleum ether:dichloromethane=5/1 (˜200 mL), and filtered to give 16.0 g of crude compound 21 as a white solid.

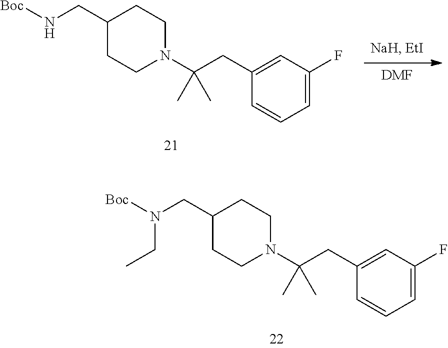

Procedure for the preparation of compound 22: A mixture of compound 21 (15.3 g, 14.0 mmol, 1.0 eq) in 10 mL of DMF was added NaH (2.8 g, 70.0 mmol, 60% purity, 5.0 eq) at 0° C. and stirred at 25° C. for 15 min. EtI (10.9 g, 70.0 mmol, 5.0 eq) was added and the mixture was stirred at 25° C. for 45 min under N₂ atmosphere. The reaction was monitored by TLC and allowed to run until completion. The reaction mixture was quenched by adding 400 mL of icy saturated aqueous NH₄Cl, then the mixture was extracted with three 200 mL portions of ethyl acetate. The combined organic layers were washed with 500 mL of brine, dried over Na₂SO₄, filtered and concentrated under reduced pressure to give 17.0 g of compound 22 as a yellow oil.

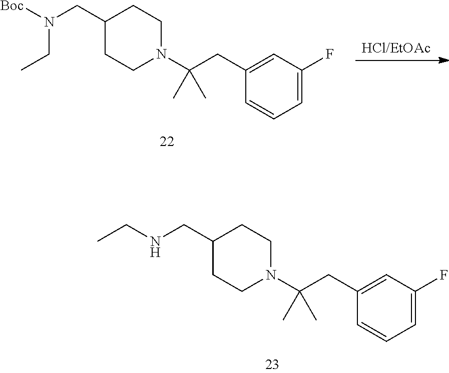 [SMILES: CCN(CC1CCN(C(C)(C)Cc2cccc(F)c2)CC1)C(=O)OC(C)(C)C ; "HCl/EtOAc" ; CCNCC1CCN(C(C)(C)Cc2cccc(F)c2)CC1]

Procedure for the preparation of compound 23: A mixture of compound 22 (17.0 g, 43.3 mmol, 1.0 eq) in HCl/ethyl acetate (200 mL, 4M) was stirred at 25° C. for 0.5 hour. The reaction was monitored by TLC and allowed to run until complete. The reaction mixture was concentrated under reduced pressure to give an oil. The oil was washed by the mixture of petroleum ether/ethyl acetate=5/1 (120 mL), and filtered to give 10.0 g of compound 23 as a light yellow solid (HCl salt).

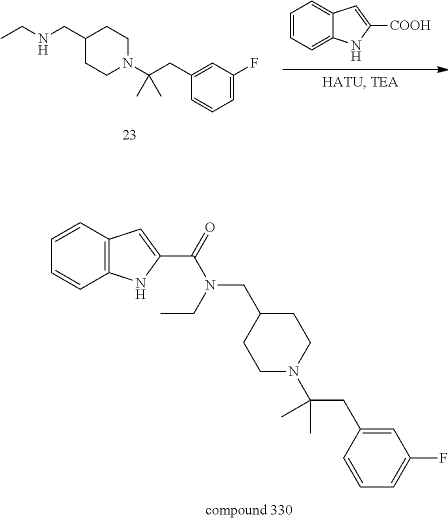

Procedure for the preparation of compound 330: A mixture of 1H-indole-2-carboxylic acid (2.7 g, 16.4 mmol, 1.0 eq), HATU (6.2 g, 16.4 mmol, 1.0 eq), and TEA (3.3 g, 32.8 mmol, 2.0 eq) in 60 mL of DMF was stirred at 25° C. for 0.5 hour, then compound 23 (6.0 g, 16.4 mmol, 1.0 eq) and TEA (3.3 g, 32.8 mmol, 2.0 eq) was then added in the mixture and the mixture was stirred at 25° C. for 11 hours. The reaction was monitored by TLC and allowed to run until complete. The reaction mixture was poured into 400 mL of ice-water (400 mL) forming some solid precipitates. The mixture was filtered to get the crude filter cake. The residue was dissolved in 100 mL of ethyl acetate. The solids were washed with 150 mL of petroleum ether and stirred at 25° C. for 5 min, and then the mixture was filtered. The solid was dissolved in HCl/ethyl acetate (100 mL, 4M). Some MeOH and ethyl acetate was added and the mixture was stirred at 25° C. for 0.5 hour. The mixture was filtered and the clear filtrate was concentrated under reduced pressure to give a solid. The solid was washed three times with 100 mL of a 5/1 mixture of ethyl acetate/methanol to give 5.69 g (73%) of compound 330 as a white solid (HCl salt).

¹H NMR (400 MHz, METHANOL-d₄) δ ppm 7.63 (d, J=7.9 Hz, 1H) 7.44 (d, J=8.3 Hz, 1H) 7.39-7.32 (m, 1H) 7.22 (t, J=7.7 Hz, 1H) 7.11-7.01 (m, 4H) 6.87 (s, 1H) 3.86-3.53 (m, 6H) 3.17-3.02 (m, 4H) 2.26-2.03 (m, 3H) 1.69 (br s, 2H) 1.36-1.27 (m, 9H)

LCMS (ESI+): m/z 436.1 (M+H)

The following compounds were prepared analogously

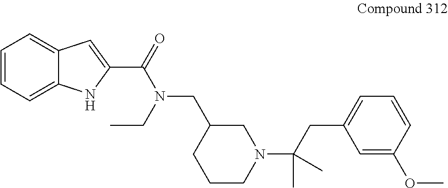 [SMILES: CCN(CC1CCCN(C(C)(C)Cc2cccc(OC)c2)C1)C(=O)c1cc2ccccc2[nH]1]

¹H NMR (400 MHz, METHANOL-d₄) δ ppm 7.59-7.70 (m, 1H) 7.40-7.47 (m, 1H) 7.18-7.27 (m, 2H) 7.07 (br t, J=7.45 Hz, 1H) 6.91 (s, 1H) 6.78-6.88 (m, 3H) 3.41-3.97 (m, 8H) 2.66-3.14 (m, 4H) 1.66-2.46 (m, 4H) 1.20-1.44 (m, 10H)

LCMS (ESI+): m/z 448.2 (M+H)

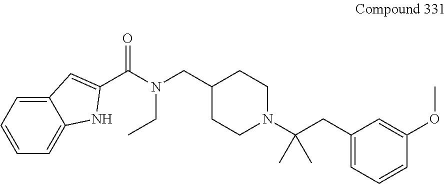 [SMILES: CCN(CC1CCN(C(C)(C)Cc2cccc(OC)c2)CC1)C(=O)c1cc2ccccc2[nH]1]

¹H NMR (400 MHz, METHANOL-d₄) δ ppm 7.60 (d, J=7.94 Hz, 1H) 7.42 (d, J=8.38 Hz, 1H) 7.13-7.23 (m, 2H) 7.04 (t, J=7.28 Hz, 1H) 6.71-6.86 (m, 4H) 3.49-3.80 (m, 7H) 2.62-2.92 (m, 4H) 1.70-2.09 (m, 5H) 1.48-1.60 (m, 1H) 1.00-1.34 (m, 10H)

LCMS (ESI+): m/z 448.2 (M+H)

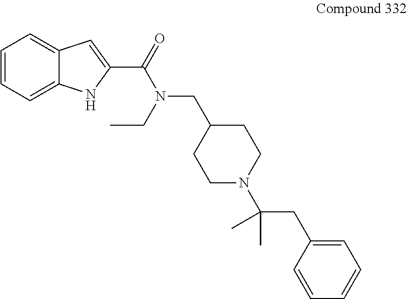 [SMILES: CCN(CC1CCN(C(C)(C)Cc2ccccc2)CC1)C(=O)c1cc2ccccc2[nH]1]

¹H NMR (400 MHz, METHANOL-d₄) δ ppm 7.63 (d, J=7.94 Hz, 1H) 7.44 (d, J=8.38 Hz, 1H) 7.20-7.37 (m, 6H) 7.07 (td, J=7.50, 0.88 Hz, 1H) 6.84-6.91 (m, 1H) 3.80 (br d, J=11.47 Hz, 4H) 3.55-3.70 (m, 2H) 3.02-3.17 (m, 4H) 2.02-2.29 (m, 3H) 1.66 (br s, 2H) 1.28-1.37 (m, 9H)

LCMS (ESI+): m/z 418.2 (M+H)

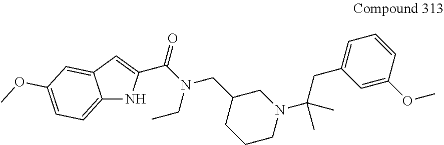

¹H NMR (400 MHz, METHANOL-d₄) δ (400 MHz, METHANOL-d₄) δ ppm 7.32-7.37 (m, 1H) 7.22-7.29 (m, 1H) 7.10 (d, J=2.43 Hz, 1H) 6.79-6.93 (m, 5H) 3.77-3.82 (m, 8H) 3.74 (br s, 2H) 3.61-3.70 (m, 1H) 2.96-3.10 (m, 3H) 2.37 (br s, 1H) 2.04-2.27 (m, 2H) 1.97 (br d, J=13.45 Hz, 1H) 1.73-1.92 (m, 2H) 1.29-1.41 (m, 10H)

LCMS (ESI+): m/z 478.2 (M+H)

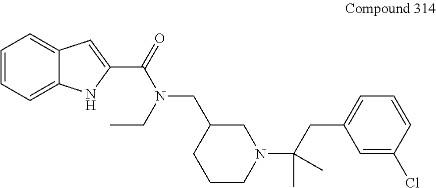 [SMILES: CCN(CC1CCCN(C(C)(C)Cc2cccc(Cl)c2)C1)C(=O)c1cc2ccccc2[nH]1]

¹H NMR (400 MHz, METHANOL-d₄) δ ppm 7.63 (d, J=8.16 Hz, 1H) 7.41-7.48 (m, 1H) 7.28-7.34 (m, 3H) 7.14-7.26 (m, 2H) 7.03-7.10 (m, 1H) 6.90 (s, 1H) 3.37-4.02 (m, 6H) 2.80-3.10 (m, 4H) 2.39 (br s, 1H) 2.11 (br d, J=14.77 Hz, 1H) 1.96 (br d, J=12.57 Hz, 1H) 1.72-1.88 (m, 1H) 1.24-1.40 (m, 10H)

LCMS (ESI+): m/z 452.3 (M+H)

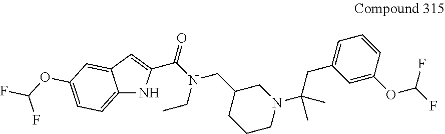 [SMILES: CCN(CC1CCCN(C(C)(C)Cc2cccc(OC(F)F)c2)C1)C(=O)c1cc2cc(OC(F)F)ccc2[nH]1]

¹H NMR (400 MHz, METHANOL-d₄) δ ppm 7.45 (d, J=8.82 Hz, 1H) 7.41 (d, J=2.21 Hz, 1H) 7.37 (t, J=7.94 Hz, 1H) 7.14 (br d, J=7.28 Hz, 1H) 6.99-7.12 (m, 4H) 6.89-6.92 (m, 1H) 6.69-6.84 (m, 1H) 3.70-3.87 (m, 4H) 3.07 (br s, 2H) 2.92 (br s, 1H) 2.37 (br d, J=9.70 Hz, 2H) 2.12 (br d, J=13.23 Hz, 2H) 1.97 (br d, J=13.01 Hz, 2H) 1.75-1.90 (m, 2H) 1.31-1.37 (m, 9H)

LCMS (ESI+): m/z 550.2 (M+H)

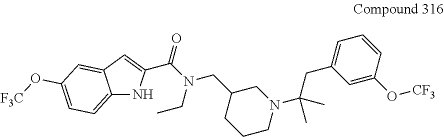 [SMILES: CCN(CC1CCCN(C(C)(C)Cc2cccc(OC(F)(F)F)c2)C1)C(=O)c1cc2cc(OC(F)(F)F)ccc2[nH]1]

¹H NMR (400 MHz, METHANOL-d₄) δ ppm 7.40-7.56 (m, 3H) 7.19-7.30 (m, 3H) 7.12 (br d, J=9.04 Hz, 1H) 6.94 (s, 1H) 3.65-3.87 (m, 4H) 3.33-3.56 (m, 2H) 3.19 (q, J=7.42 Hz, 1H) 3.04-3.12 (m, 2H) 2.90 (br s, 1H) 2.35 (br d, J=8.38 Hz, 1H) 2.11 (br d, J=13.89 Hz, 1H) 1.91-2.01 (m, 1H) 1.76-1.89 (m, 1H) 1.20-1.39 (m, 12H)

LCMS (ESI+): m/z 586.2 (M+H)

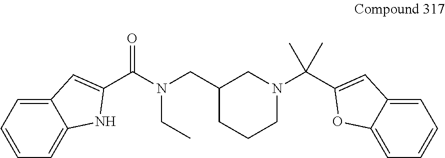 [SMILES: CCN(CC1CCCN(C(C)(C)c2cc3ccccc3o2)C1)C(=O)c1cc2ccccc2[nH]1]

¹H NMR (400 MHz, METHANOL-d₄) δ ppm 7.49-7.56 (m, 2H) 7.42 (dd, J=8.16, 2.20 Hz, 2H) 7.11-7.29 (m, 4H) 7.03-7.11 (m, 2H) 3.57-3.71 (m, 4H) 3.33-3.43 (m, 2H) 3.29-3.51 (m, 3H) 3.10 (br d, J=12.13 Hz, 1H) 2.91-3.02 (m, 1H) 2.34 (br s, 1H) 2.04 (br d, J=14.33 Hz, 1H) 1.87 (d, J=8.82 Hz, 6H) 1.26-1.32 (m, 1H) 1.17 (t, J=7.17 Hz, 3H)

LCMS (ESI+): m/z 444.1 (M+H)

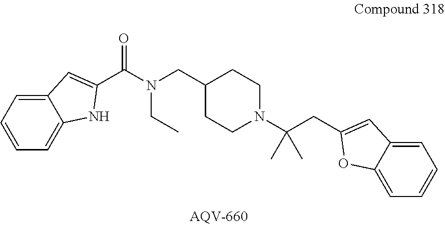 [SMILES: CCN(CC1CCN(C(C)(C)Cc2cc3ccccc3o2)CC1)C(=O)c1cc2ccccc2[nH]1]

¹H NMR (400 MHz, METHANOL-d₄) δ ppm 7.61 (d, J=8.16 Hz, 1H) 7.55 (d, J=7.94 Hz, 1H) 7.39-7.47 (m, 2H) 7.18-7.30 (m, 1H) 7.18-7.30 (m, 2H) 7.04-7.09 (m, 1H) 6.86 (s, 1H) 6.75 (s, 1H) 3.81 (br d, J=11.03 Hz, 4H) 3.61 (br s, 2H) 3.32-3.39 (m, 1H) 3.12 (br t, J=12.24 Hz, 2H) 2.03-2.25 (m, 2H) 1.48 (s, 8H) 1.34 (br t, J=7.06 Hz, 3H)

LCMS (ESI+): m/z 458.2 (M+H)

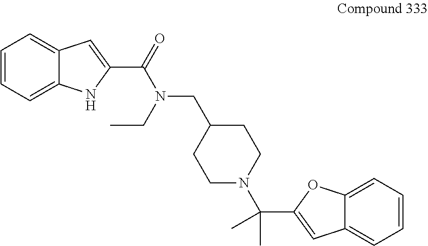 [SMILES: CCN(CC1CCN(C(C)(C)c2cc3ccccc3o2)CC1)C(=O)c1cc2ccccc2[nH]1]

¹H NMR (400 MHz, METHANOL-d₄) δ ppm 7.65 (d, J=7.7 Hz, 1H), 7.58 (d, J=7.9 Hz, 1H), 7.54 (br d, J=8.6 Hz, 1H), 7.36-7.42 (m, 2H), 7.27-7.31 (m, 1H), 7.27-7.32 (m, 1H), 7.19 (t, J=7.6 Hz, 1H), 7.11 (s, 1H), 7.04 (t, J=7.5 Hz, 1H), 6.81 (br s, 1H), 3.73 (br s, 2H), 3.49 (br d, J=12.3 Hz, 4H), 2.99 (br t, J=11.8 Hz, 2H), 1.99 (br d, J=14.6 Hz, 1H), 1.87 (s, 6H), 1.58 (br s, 3H), 1.21-1.32 (m, 4H)

LCMS (ESI+): m/z 444.1 (M+H)

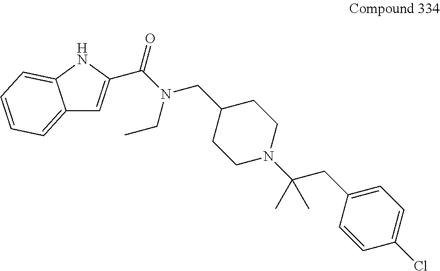 [SMILES: CCN(CC1CCN(C(C)(C)Cc2ccc(Cl)cc2)CC1)C(=O)c1cc2ccccc2[nH]1]

¹H NMR (400 MHz, METHANOL-d₄) δ ppm 7.62 (d, J=8.16 Hz, 1H) 7.44 (d, J=7.50 Hz, 1H) 7.32-7.38 (m, 2H) 7.19-7.28 (m, 3H) 7.07 (td, J=7.50, 0.88 Hz, 1H) 6.87 (s, 1H) 3.79 (br d, J=11.91 Hz, 4H) 3.63 (br s, 2H) 3.13 (br t, J=12.90 Hz, 2H) 3.03 (s, 2H) 2.05-2.23 (m, 3H) 1.28-1.37 (m, 10H)

LCMS (ESI+): m/z 452.2 (M+H)

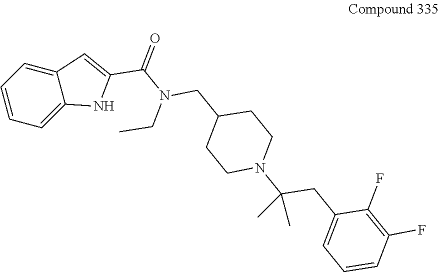 [SMILES: CCN(CC1CCN(C(C)(C)Cc2cccc(F)c2F)CC1)C(=O)c1cc2ccccc2[nH]1]

¹H NMR (400 MHz, METHANOL-d₄) δ ppm 7.62 (d, J=7.9 Hz, 1H), 7.44 (dd, J=8.4, 0.9 Hz, 1H), 7.11-7.30 (m, 4H), 7.07 (ddd, J=8.0, 7.1, 1.0 Hz, 1H), 6.87 (s, 1H), 3.81 (br d, J=11.5 Hz, 4H), 3.61 (br s, 2H), 3.06-3.20 (m, 4H), 2.21 (br s, 1H), 2.11 (br d, J=13.5 Hz, 2H), 1.60 (br s, 2H), 1.24-1.39 (m, 9H)

LCMS (ESI+): m/z 454.2 (M+H)

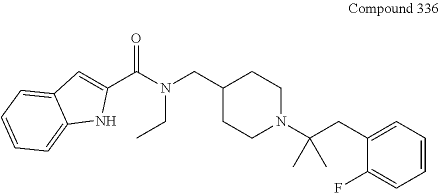 [SMILES: CCN(CC1CCN(C(C)(C)Cc2ccccc2F)CC1)C(=O)c1cc2ccccc2[nH]1]

¹H NMR (400 MHz, METHANOL-d₄) δ ppm 7.63 (br d, J=7.9 Hz, 1H), 7.45 (d, J=8.2 Hz, 1H), 7.34 (qd, J=7.0, 13.7 Hz, 2H), 7.27-7.11 (m, 3H), 7.10-7.05 (m, 1H), 6.92-6.85 (m, 1H), 3.91-3.45 (m, 5H), 3.23-3.01 (m, 3H), 2.31-1.95 (m, 3H), 1.78-1.46 (m, 2H), 1.42-1.11 (m, 11H)

LCMS (ESI+): m/z 436.1 (M+H)

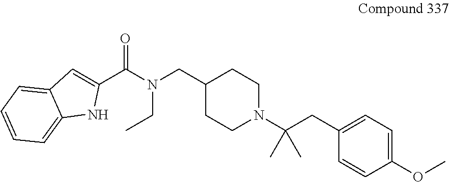 [SMILES: CCN(CC1CCN(C(C)(C)Cc2ccc(OC)cc2)CC1)C(=O)c1cc2ccccc2[nH]1]

¹H NMR (400 MHz, METHANOL-d₄) δ ppm 7.63 (d, J=7.94 Hz, 1H) 7.44 (br d, J=8.16 Hz, 1H) 7.13-7.26 (m, 3H) 7.08 (t, J=7.02 Hz, 1H) 6.81-6.95 (m, 3H) 3.78 (s, 6H) 3.59 (s, 1H) 2.92-3.16 (m, 4H) 2.16-2.26 (m, 1H) 2.00-2.16 (m, 2H) 1.48-1.64 (m, 2H) 1.25-1.38 (m, 9H)

LCMS (ESI+): m/z 448.2 (M+H)

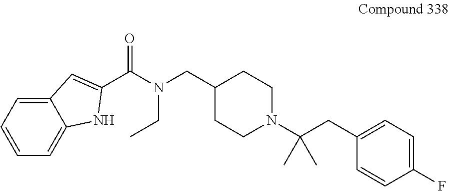 [SMILES: CCN(CC1CCN(C(C)(C)Cc2ccc(F)cc2)CC1)C(=O)c1cc2ccccc2[nH]1]

¹H NMR (400 MHz, METHANOL-d₄) δ ppm 7.64 (d, J=7.9 Hz, 1H), 7.48-7.44 (m, 1H), 7.30 (dd, J=5.4, 8.3 Hz, 2H), 7.24 (dt, J=1.1, 7.7 Hz, 1H), 7.13-7.07 (m, 3H), 6.93-6.87 (m, 1H), 3.88-3.60 (m, 6H), 3.19-3.03 (m, 4H), 2.27-2.09 (m, 3H), 1.62 (br s, 2H), 1.39-1.31 (m, 9H)

LCMS (ESI+): m/z 436.1 (M+H)

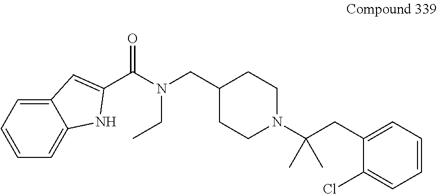 [SMILES: CCN(CC1CCN(C(C)(C)Cc2ccccc2Cl)CC1)C(=O)c1cc2ccccc2[nH]1]

¹H NMR (400 MHz, METHANOL-d₄) δ (400 MHz, METHANOL-d) δ ppm 7.61 (d, J=7.94 Hz, 1H) 7.40-7.47 (m, 2H) 7.36 (br d, J=4.41 Hz, 1H) 7.27-7.32 (m, 2H) 7.20 (t, J=7.72 Hz, 1 H) 7.03-7.09 (m, 1H) 6.86 (s, 1H) 3.81 (br d, J=11.25 Hz, 3H) 3.61 (br s, 1H) 3.25 (br s, 2H) 3.13-3.21 (m, 4H) 2.04-2.25 (m, 3H) 1.61 (br s, 1H) 1.28-1.35 (m, 10H)

LCMS (ESI+): m/z 452.1 (M+H)

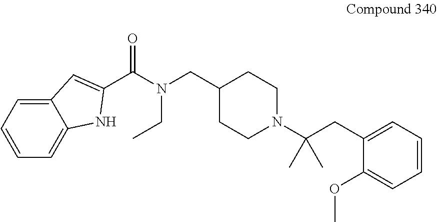 [SMILES: CCN(CC1CCN(C(C)(C)Cc2ccccc2OC)CC1)C(=O)c1cc2ccccc2[nH]1]

¹H NMR (400 MHz, METHANOL-d₄) δ (400 MHz, METHANOL-d₄) δ ppm 7.59-7.65 (m, 1H) 7.45 (d, J=8.38 Hz, 1H) 7.13-7.33 (m, 3H) 6.84-7.11 (m, 4H) 3.67-3.90 (m, 5H) 3.58 (br s, 1H) 2.99-3.14 (m, 4H) 2.14-2.27 (m, 1H) 2.07 (br d, J=13.45 Hz, 2H) 1.55-1.78 (m, 2H) 1.24-1.43 (m, 12H)

LCMS (ESI+): m/z 448.3 (M+H)

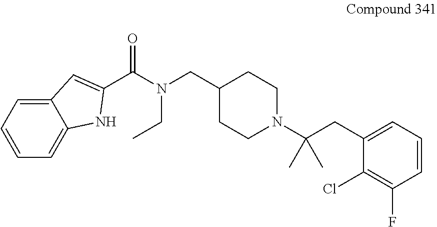 [SMILES: CCN(CC1CCN(C(C)(C)Cc2cccc(F)c2Cl)CC1)C(=O)c1cc2ccccc2[nH]1]

¹H NMR (400 MHz, METHANOL-d₄) δ (400 MHz, METHANOL-d₄) δ ppm 7.61 (d, J=7.72 Hz, 1H) 7.42 (d, J=8.38 Hz, 1H) 7.29-7.36 (m, 1H) 7.17-7.24 (m, 3H) 7.05 (t, J=7.50 Hz, 1H) 6.86 (s, 1H) 3.81 (br d, J=11.03 Hz, 4H) 3.61 (br s, 2H) 3.17 (br t, J=12.68 Hz, 2H) 2.21 (br s, 1H) 2.10 (br d, J=13.23 Hz, 2H) 1.62 (br s, 2H) 1.28-1.39 (m, 10H)

LCMS (ESI+): m/z 470.1 (M+H)

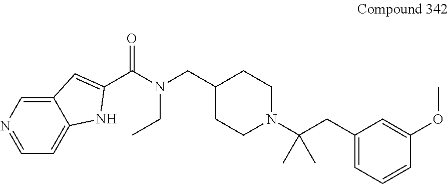 [SMILES: CCN(CC1CCN(C(C)(C)Cc2cccc(OC)c2)CC1)C(=O)c1cc2cnccc2[nH]1]

¹H NMR (400 MHz, METHANOL-d₄) δ (400 MHz, METHANOL-d) δ ppm 9.23 (s, 1H) 8.39 (d, J=6.84 Hz, 1H) 7.95 (d, J=6.61 Hz, 1H) 7.41 (br s, 1H) 7.24 (br t, J=7.83 Hz, 1H) 6.76-6.89 (m, 3H) 3.77 (s, 6H) 3.60 (br s, 2H) 3.13 (br t, J=11.80 Hz, 2H) 3.00 (br s, 2H) 2.11 (br s, 3H) 1.64 (br s, 2H) 1.32 (br s, 10H)

LCMS (ESI+): m/z 225.2 (M/2+H)

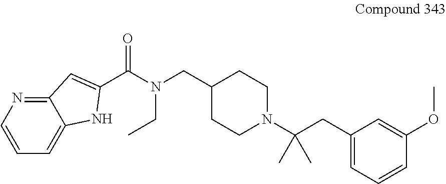 [SMILES: CCN(CC1CCN(C(C)(C)Cc2cccc(OC)c2)CC1)C(=O)c1cc2ncccc2[nH]1]

¹H NMR (400 MHz, METHANOL-d₄) δ (400 MHz, METHANOL-d₄) δ ppm 8.68 (dd, J=5.84, 0.99 Hz, 1H) 8.63 (d, J=8.38 Hz, 1H) 7.79 (dd, J=8.38, 5.73 Hz, 1H) 7.25 (br d, J=7.94 Hz, 1H) 7.15 (s, 1H) 6.75-6.90 (m, 3H) 3.79 (s, 5H) 3.64-3.71 (m, 2H) 3.60 (d, J=7.28 Hz, 2H) 3.08-3.20 (m, 2H) 2.94-3.05 (m, 2H) 2.05-2.28 (m, 3H) 1.70 (br d, J=11.91 Hz, 2H) 1.29-1.37 (m, 9H)

LCMS (ESI+): m/z 225.2 (M/2+H)

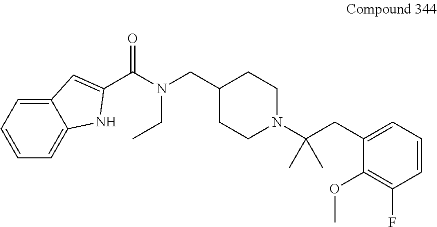 [SMILES: CCN(CC1CCN(C(C)(C)Cc2cccc(F)c2OC)CC1)C(=O)c1cc2ccccc2[nH]1]

¹H NMR (400 MHz, METHANOL-d₄) δ (400 MHz, METHANOL-d₄) δ ppm 7.62 (d, J=7.89 Hz, 1H) 7.44 (d, J=8.33 Hz, 1H) 7.22 (t, J=7.67 Hz, 1H) 6.99-7.14 (m, 4H) 6.87 (s, 1H) 3.94 (s, 3H) 3.80 (br d, J=10.52 Hz, 4H) 3.61 (br s, 2H) 3.04-3.17 (m, 4H) 2.05-2.25 (m, 3H) 1.59 (br s, 1H) 1.29-1.39 (m, 9H)

LCMS (ESI+): m/z 466.4 (M+H)

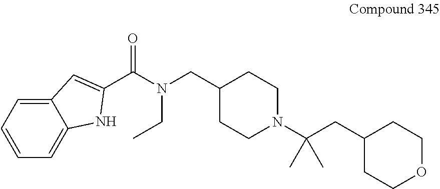 [SMILES: CCN(CC1CCN(C(C)(C)CC2CCOCC2)CC1)C(=O)c1cc2ccccc2[nH]1]

¹H NMR (400 MHz, METHANOL-d₄) δ (400 MHz, METHANOL-d) δ ppm 7.62 (d, J=7.94 Hz, 1H) 7.44 (d, J=8.38 Hz, 1H) 7.22 (t, J=7.72 Hz, 1H) 7.03-7.11 (m, 1H) 6.86 (br s, 1H) 3.89 (br dd, J=11.25, 3.09 Hz, 2H) 3.79 (br s, 2H) 3.49-3.72 (m, 4H) 3.43 (br t, J=11.80 Hz, 2H) 2.99 (br t, J=12.79 Hz, 2H) 2.15 (br s, 1H) 2.05 (br d, J=13.89 Hz, 2H) 1.59-1.77 (m, 6H) 1.43 (br s, 6H) 1.30-1.40 (m, 4H)

LCMS (ESI+): m/z 426.4 (M+H)

### Example 21: General Protocol R for Synthesis of Exemplary Compounds

General Protocol R to synthesize exemplary compounds of Formula (I) is described in Scheme 18 and the procedures set forth below.

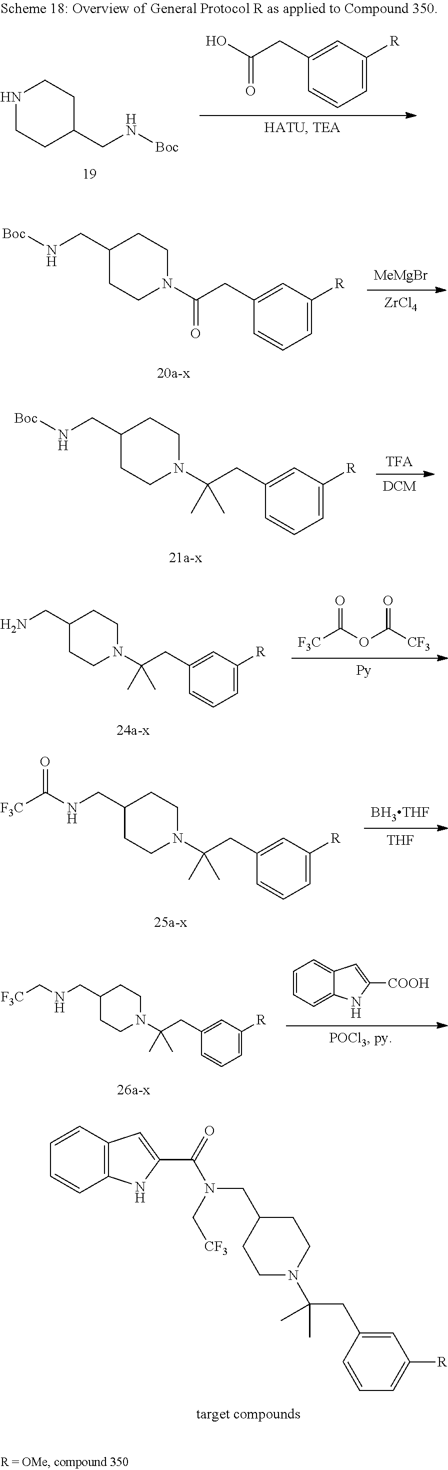

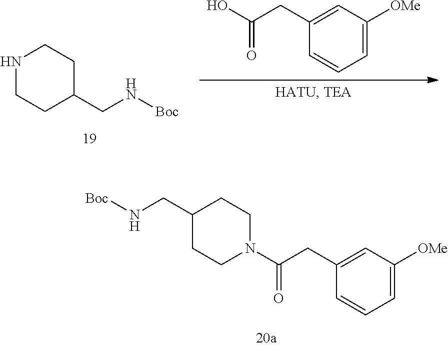

Procedure for the preparation of amide 20a: The mixture of compound 19 (600 mg, 2.8 mmol, 1.0 eq), 2-(3-methoxyphenyl)acetic acid (465 mg, 2.8 mmol, 1.0 eq) and HATU (1.1 g, 2.8 mmol, 1.0 eq) in 2 mL of DMF was added Et₃N (708 mg, 7.0 mmol, 2.5 eq) in one portion at 25° C. The mixture was stirred at 25° C. for 2 hrs. The reaction was monitored by TLC and allowed to run until completion. The mixture was poured into 30 mL of ice-water and extracted with three 10 mL portions of ethyl acetate. The combined organic phase was washed twice with 20 mL of brine, dried with anhydrous Na₂SO₄. The residue was purified by flash silica gel chromatography (ISCO®; 20 g SepaFlash® Silica Flash Column, Eluent of 0-60% ethyl acetate/petroleum ether gradient @ 70 mL/min) to give 1.2 g of compound 20a as a colorless oil.

The procedure above can be used generally to produce similar amides.

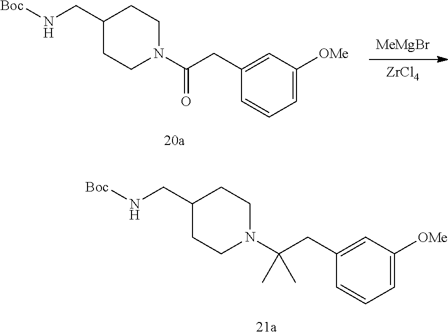 [SMILES: "MeMgBr" ; COc1cccc(CC(=O)N2CCC(CNC(=O)OC(C)(C)C)CC2)c1 ; "ZrCl4" ; COc1cccc(CC(C)(C)N2CCC(CNC(=O)OC(C)(C)C)CC2)c1]

Procedure for the preparation of gem dimethyl intermediate 21a: To a solution of compound 20a (1.2 g, 3.3 mmol, 1.0 eq) in 12 mL of THF was added ZrCl₄ (849 mg, 3.6 mmol, 1.1 eq) at −10° C. The mixture was stirred at −10° C. for 1 hr. Then MeMgBr (3M, 7.7 mL, 7.0 eq) was added dropwise at −10° C. The reaction mixture was warmed to 25° C. and stirred for 4 hrs. The reaction was monitored by TLC and allowed to run until completion. The mixture was poured into 50 mL of ice aq. NH₄Cl and then 10 mL of 1N aqueous HCl was added HCl. The mixture was extracted with three 20 mL portions of ethyl acetate. The combined organic phase was washed with 50 mL of brine, dried with anhydrous Na₂SO₄, filtered and concentrated in vacuum to give 700 mg (56%) of compound 21a as a yellow solid.

The procedure above can be used analogously to prepare related compounds 21

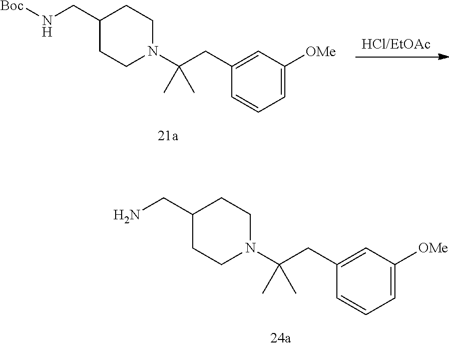 [SMILES: COc1cccc(CC(C)(C)N2CCC(CNC(=O)OC(C)(C)C)CC2)c1 ; "HCl/EtOAc" ; COc1cccc(CC(C)(C)N2CCC(CN)CC2)c1]

Procedure for the preparation of compound 24a: The mixture of compound 21a (700 mg, 1.9 mmol, 1.0 eq) in 10 mL of 4M HCl in ethyl acetate was stirred at 25° C. for 1 hr. The reaction was monitored by TLC and allowed to run until complete. The reaction was concentrated in vacuum to give 450 mg of compound 24a (HCl salt) as a yellow oil.

The procedure above can be used analogously to prepare related compounds 24.

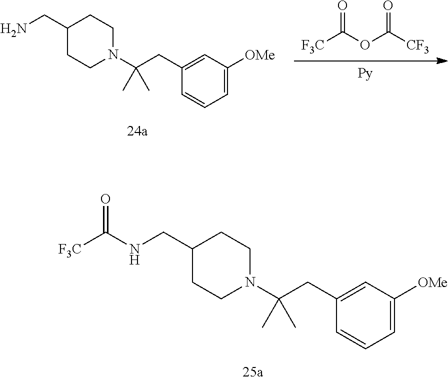

Procedure for the preparation of compound 25a: To a mixture of compound 24a (450 mg, 1.4 mmol, 1.0 eq, HCl salt), TEA (723 mg, 7.1 mmol, 3.0 eq) in 8 mL of pyridine was added (2, 2, 2-trifluoroacetyl) 2, 2, 2-trifluoroacetate (363 mg, 1.7 mmol, 1.2 eq), the mixture was stirred at 25° C. for 2 hours under N₂ atmosphere. The reaction was monitored by LCMS and allowed to run until complete. It was evaporated under reduced pressure to give a residue which was diluted with 30 mL of ethyl acetate, washed with 30 mL of saturated aqueous NH₄Cl and 30 mL of brine, dried over Na₂SO₄, filtered and evaporated to give 400 mg of the crude trifluoroacetate 25a as an orange oil.

The procedure above can be used analogously to prepare related compounds 25.

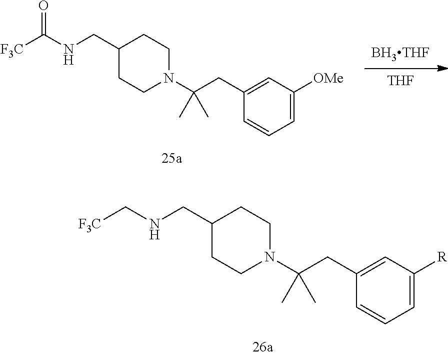

Procedure for the preparation of compound 26a: A mixture of compound 25a (400 mg, 1.1 mmol, 1.0 eq) in 5 mL of THF was degassed and purged with N₂ 3 times. To the mixture was added BH₃.THF (1 M, 3.2 mL, 3.0 eq) dropwise at 0° C. The mixture was stirred at 70° C. for 3 hours under N₂ atmosphere. The reaction was monitored by LCMS and allowed to run until complete. It was quenched by adding 10 mL of MeOH slowly, evaporated under reduced pressure to give the crude product which was partitioned between 20 mL of saturated aqueous NaHCO₃ and 25 mL of ethyl acetate. The organic phase was separated, washed with 20 mL of brine, dried over Na₂SO₄, filtered and concentrated under reduced pressure to give 300 mg of compound 26a as a colorless oil.

The procedure above can be used analogously to prepare related compounds 26.

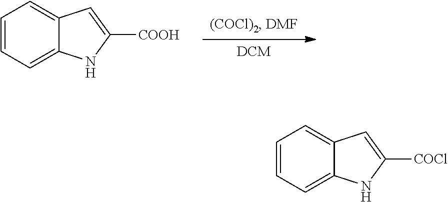

Procedure for the preparation of acid chloride: A mixture of indole-2-carboxylic acid (200 mg, 1.2 mmol, 1.0 eq), oxalyl chloride (473 mg, 3.7 mmol, 3.0 eq), DMF (9.1 mg, 124.0 μmol, 0.1 eq) in 6b mL of DCM was degassed and purged with N₂ 3 times. The mixture was stirred at 25° C. for 0.5 hour under N₂ atmosphere. The reaction was monitored by TLC and allowed to run until complete. It was evaporated under reduced pressure to give the crude acid chloride (230 mg) as yellow gum and to be used into the next step without further purification.

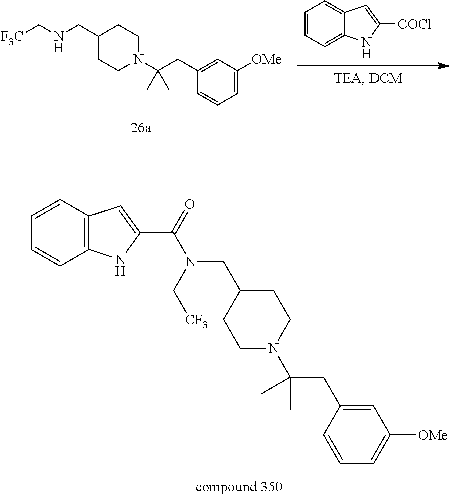

Procedure for the preparation of compound 350: A mixture of trifluoroethyl amine 26a (50.0 mg, 139.5 μmol, 1.0 eq), Et₃N (42.3 mg, 418.5 μmol, 3.0 eq) in 1 mL of DCM was added 1H-indole-2-carbonyl chloride (25.1 mg, 139.5 μmol, 1.0 eq) at 0° C., then the mixture was stirred at 25° C. for 1 hour under N₂ atmosphere. The reaction was monitored by LCMS and allowed to run until complete. The reaction mixture was quenched by adding 20 mL of aqueous NH₄Cl and extracted with three 10 mL portions of DCM. The combined organic phase was washed with 30 mL of brine, dried with anhydrous Na₂SO₄, filtered and concentrated in vacuum. The residue was purified by prep-HPLC (TFA condition) to give 22.5 mg (25%) of the TFA salt of compound 350 as a violet solid.

¹H NMR (400 MHz, METHANOL-d₄) δ ppm 7.64 (d, J=7.94 Hz, 1H) 7.45 (d, J=8.16 Hz, 1H) 7.25 (t, J=7.83 Hz, 2H) 7.09 (t, J=7.50 Hz, 1H) 6.93 (s, 1H) 6.87 (br d, J=7.94 Hz, 1H) 6.77-6.83 (m, 2H) 4.51 (br d, J=8.60 Hz, 2H) 3.71-3.89 (m, 7H) 3.08 (br t, J=12.57 Hz, 2H) 2.97 (s, 2H) 2.13-2.26 (m, 1H) 2.05 (br d, J=13.45 Hz, 2H) 1.35-1.62 (m, 2H) 1.29 (s, 6H)

LCMS (ESI+): m/z 502.3 (M+H)

The following compounds were prepared analogously using General Protocol R:

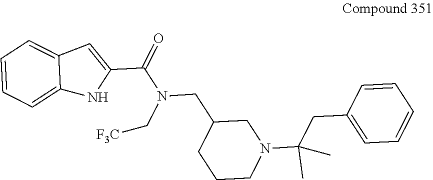 [SMILES: CC(C)(Cc1ccccc1)N1CCCC(CN(CC(F)(F)F)C(=O)c2cc3ccccc3[nH]2)C1]

¹H NMR (400 MHz, METHANOL-d₄) δ ppm 7.65 (d, J=7.94 Hz, 1H) 7.46 (dd, J=8.27, 0.77 Hz, 1H) 7.20-7.35 (m, 6H) 7.09 (td, J=7.61, 0.88 Hz, 1H) 7.07-7.12 (m, 1H) 6.97 (s, 1H) 6.95-6.98 (m, 1H) 4.55 (q, J=8.45 Hz, 2H) 3.92 (br d, J=5.51 Hz, 1H) 3.73 (br d, J=10.80 Hz, 2H) 3.61 (br d, J=11.03 Hz, 1H) 2.95-3.08 (m, 3H) 2.84 (br t, J=11.91 Hz, 1H) 2.37 (br s, 1H) 2.09 (br d, J=14.55 Hz, 1H) 1.94 (br d, J=14.11 Hz, 1H) 1.79 (q, J=13.60 Hz, 1H) 1.28 (s, 7H)

LCMS (ESI+): m/z 472.2 (M+H)

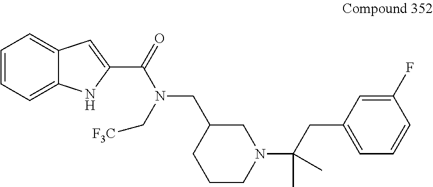 [SMILES: CC(C)(Cc1cccc(F)c1)N1CCCC(CN(CC(F)(F)F)C(=O)c2cc3ccccc3[nH]2)C1]

¹H NMR (400 MHz, METHANOL-d₄) δ ppm 7.65 (d, J=7.72 Hz, 1H) 7.46 (d, J=8.38 Hz, 1H) 7.23-7.40 (m, 2H) 6.92-7.16 (m, 5H) 4.56 (br d, J=9.26 Hz, 2H) 3.92 (s, 1H) 3.73 (br d, J=12.79 Hz, 2H) 3.61 (br d, J=11.03 Hz, 1H) 3.01 (br s, 2H) 2.85 (br s, 2H) 2.37 (br s, 1H) 1.93 (br s, 2H) 1.30 (s, 8H)

LCMS (ESI+): m/z 490.3 (M+H)

### Example 22: General Protocol S for Synthesis of Exemplary Compounds

General Protocol S to synthesize exemplary compounds of Formula (I) is described in Scheme 19 and the procedures set forth below.

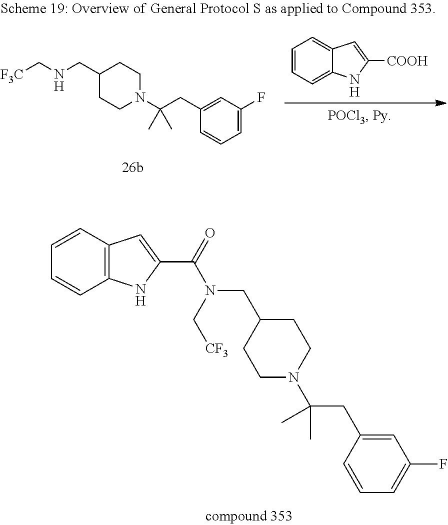

Procedure for the preparation of compound 353: A mixture of 1H-indole-2-carboxylic acid (50.0 mg, 310.3 μmol, 1.0 eq), compound 26b (107.5 mg, 310.3 μmol, 1.0 eq) in 2 mL of pyridine was cooled to 0° C. POCl₃ (71.4 mg, 465.4 μmol, 1.5 eq) was added slowly, then the mixture was stirred at 0° C. for 1 hour under N₂ atmosphere. The reaction was monitored by LCMS and allowed to run until complete. It was evaporated under reduced pressure to give a residue which was partitioned between 10 mL of saturated aqueous NH₄Cl and 10 mL of ethyl acetate. The organic phase was separated, washed with 10 mL of brine, dried over Na₂SO₄, filtered and concentrated under reduced pressure to give a residue. It was purified by prep-TLC (eluting with petroleum ether/ethyl acetate=1/1). Then it was re-purified by prep-HPLC (neutral condition) to give 2.4 mg (1.5%) compound 353 as a light brown gum.

¹H NMR (400 MHz, METHANOL-d₄) δ ppm 7.56 (d, J=8.16 Hz, 1H) 7.37 (dd, J=8.27, 0.77 Hz, 1H) 7.11-7.19 (m, 2H) 7.01 (ddd, J=8.05, 7.06, 0.99 Hz, 1H) 6.79-6.90 (m, 4H) 4.50 (br s, 3H) 4.38 (br d, J=8.82 Hz, 2H) 3.74 (br s, 1H) 3.08 (br d, J=15.21 Hz, 1H) 2.61-2.73 (m, 2H) 2.28 (br s, 1H) 1.76-1.86 (m, 1H) 1.82 (br s, 1H) 1.69 (br d, J=11.03 Hz, 2H) 1.10-1.32 (m, 8H)

LCMS (ESI+): m/z 490.3 (M+H)

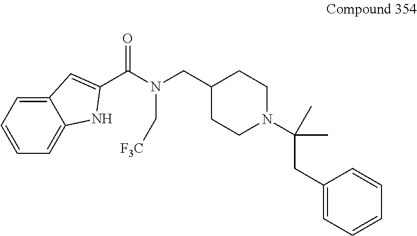 [SMILES: CC(C)(Cc1ccccc1)N1CCC(CN(CC(F)(F)F)C(=O)c2cc3ccccc3[nH]2)CC1]

¹H NMR (400 MHz, METHANOL-d₄) δ ppm 7.64 (d, J=7.94 Hz, 1H) 7.45 (d, J=8.38 Hz, 1H) 7.21-7.35 (m, 6H) 7.10 (t, J=7.50 Hz, 1H) 6.93 (s, 1H) 4.51 (br d, J=8.38 Hz, 2H) 3.86 (br s, 2H) 3.77 (br d, J=11.47 Hz, 2H) 3.10 (br t, J=12.79 Hz, 2H) 3.00 (s, 2H) 2.11-2.28 (m, 1H) 2.06 (br d, J=14.33 Hz, 2H) 1.38 (s, 2H) 1.28 (s, 6H)

LCMS (ESI+): m/z 472.2 (M+H).

### Example 23: General Protocol T for Synthesis of Exemplary Compounds

General Protocol T to synthesize exemplary compounds of Formula (I) is described in Scheme 20 and the procedures set forth below.

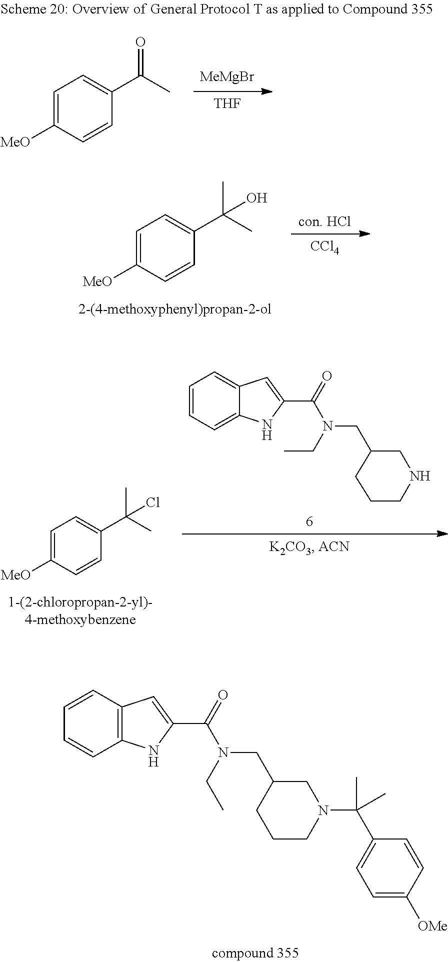

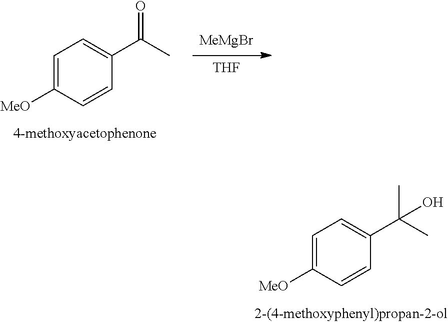

General procedure for the preparation of 2-(4-methoxyphenyl)propan-2-ol: A mixture of 4-methoxyacetophenone (2.0 g, 13.3 μmol, 1.0 eq) in 20 mL of THF was added 13.3 mL of 3M MeMgBr (3.0 eq) at 0° C., and then the mixture was stirred at 25° C. for 36 h under N₂ atmosphere. The reaction was monitored by TLC and allowed to run until complete. The reaction mixture was quenched by 40 mL of icy saturated aqueous NH₄Cl and extracted twice with 30 mL of ethyl acetate. The combined organic layers were washed twice with 50 mL of brine, dried over Na₂SO₄, filtered and concentrated under reduced pressure. The residue was purified by column chromatography (SiO₂, eluting with petroleum ether/ethyl acetate=30/1 to 8/1) to give 900 mg of 2-(4-methoxyphenyl)propan-2-ol as a yellow oil.

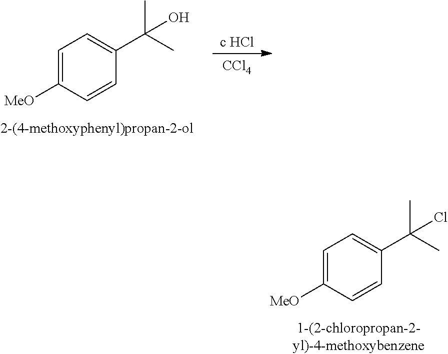

General procedure for the preparation of 1-(2-chloropropan-2-yl)-4-methoxybenzene: A mixture of 2-(4-methoxyphenyl)propan-2-ol (300 mg, 1.8 μmol, 1.0 eq) in 2.5 mL of CCl₄ was added HCl (50.00 μL, 12M) at 0° C., and then the mixture was stirred at 0° C. for 1 min. The reaction was monitored by TLC and allowed to run until complete. The organic layer was separated and the crude chloro intermediate (in CCl₄) was used into the next step without further purification.

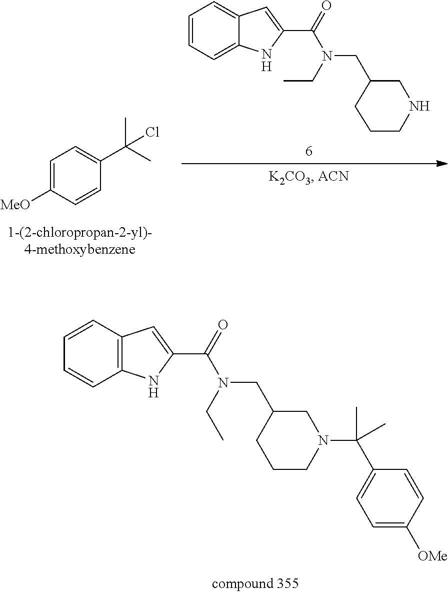

Procedure for preparation of compound 355: To a mixture of 1-(2-chloropropan-2-yl)-4-methoxybenzene (30.0 mg, 105.1 μmol, 1.0 eq), TEA (21.3 mg, 210 μmol, 2.0 eq) in 2.0 mL of ACN was added compound 6 (38.8 mg, 210.2 μmol, 2.0 eq) at 0° C., and then the mixture was stirred at 25° C. for 0.5 h. The reaction was monitored by LCMS and allowed to run until complete. The reaction mixture was filtered and the residue was purified by prep-TLC (SiO₂, eluting with petroleum ether/ethyl acetate=9/1) then prep-HPLC (TFA condition) to give 2.5 mg (4%) of the TFA salt of compound 355 as a white solid.

¹H NMR (400 MHz, METHANOL-d₄) δ ppm 7.64 (d, J=7.9 Hz, 1H), 7.56-7.45 (m, 3H), 7.25 (dt, J=1.1, 7.6 Hz, 1H), 7.12-7.07 (m, 1H), 6.88 (d, J=9.0 Hz, 2H), 6.71 (s, 1H), 3.73-3.47 (m, 7H), 3.42-3.33 (m, 1H), 3.23 (br d, J=13.0 Hz, 1H), 3.06 (br d, J=10.6 Hz, 1H), 2.86 (br s, 1H), 2.60 (br s, 1H), 2.30-2.20 (m, 1H), 2.03 (br d, J=5.3 Hz, 1H), 1.88-1.76 (m, 8H), 1.24-1.24 (m, 1H), 1.22 (t, J=7.1 Hz, 2H).

LCMS (ESI+): m/z 434.3 (M+H)

The following compounds were prepared analogously:

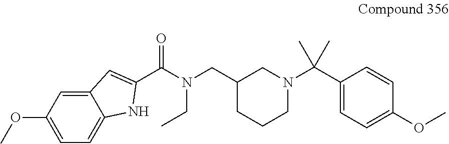

¹H NMR (400 MHz, METHANOL-d₄) δ ppm 7.50 (br d, J=6.8 Hz, 2H), 7.37 (d, J=9.0 Hz, 1H), 7.11 (d, J=2.2 Hz, 1H), 6.92 (dd, J=8.8, 2.4 Hz, 1H), 6.88 (br d, J=8.8 Hz, 2H), 6.64 (s, 1H), 3.83 (s, 3H), 3.75-3.48 (m, 7H), 3.42-3.33 (m, 1H), 3.30-3.18 (m, 1H), 3.12-3.11 (m, 1H), 3.07 (br d, J=11.9 Hz, 1H), 2.85 (br s, 1H), 2.60 (br s, 1H), 2.25 (br d, J=3.5 Hz, 1H), 2.02 (br d, J=13.9 Hz, 1H), 1.89-1.71 (m, 8H), 1.22 (t, J=7.1 Hz, 3H).

LCMS (ESI+): m/z 464.2 (M+H)

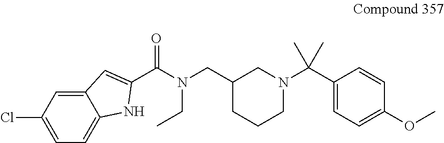 [SMILES: CCN(CC1CCCN(C(C)(C)c2ccc(OC)cc2)C1)C(=O)c1cc2cc(Cl)ccc2[nH]1]

¹H NMR (400 MHz, METHANOL-d₄) δ ppm 7.63 (d, J=1.8 Hz, 1H), 7.53-7.42 (m, 3H), 7.21 (dd, J=1.8, 8.8 Hz, 1H), 6.88 (d, J=8.8 Hz, 2H), 6.65 (s, 1H), 3.71-3.47 (m, 7H), 3.39-3.32 (m, 1H), 3.29 (br s, 1H), 3.05 (br d, J=11.0 Hz, 1H), 2.86 (br s, 1H), 2.63 (br s, 1H), 2.25 (br s, 1H), 2.02 (br d, J=7.0 Hz, 1H), 1.81 (br d, J=8.8 Hz, 8H), 1.20 (t, J=7.2 Hz, 3H).

LCMS (ESI+): m/z 468.3 (M+H)

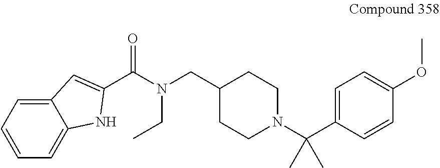 [SMILES: CCN(CC1CCN(C(C)(C)c2ccc(OC)cc2)CC1)C(=O)c1cc2ccccc2[nH]1]

¹H NMR (400 MHz, METHANOL-d₄) δ ppm 7.58 (br dd, J=8.3, 18.9 Hz, 3H), 7.42 (d, J=8.4 Hz, 1H), 7.21 (dt, J=1.0, 7.7 Hz, 1H), 7.03 (br d, J=9.0 Hz, 3H), 6.87-6.79 (m, 1H), 3.82 (s, 3H), 3.72 (br s, 2H), 3.54 (br s, 1H), 3.42 (br d, J=11.7 Hz, 2H), 2.83 (br t, J=11.9 Hz, 2H), 2.03 (br d, J=5.5 Hz, 1H), 1.99-1.91 (m, 2H), 1.82 (s, 5H), 1.60-1.47 (m, 1H), 1.30 (q, J=6.9 Hz, 6H).

LCMS (ESI+): m/z 434.3 (M+H)

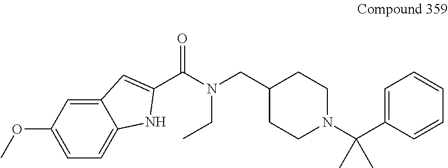 [SMILES: CCN(CC1CCN(C(C)(C)c2ccccc2)CC1)C(=O)c1cc2cc(OC)ccc2[nH]1]

¹H NMR (400 MHz, METHANOL-d₄) δ ppm 8.00 (br d, J=7.9 Hz, 2H), 7.74-7.69 (m, 1H), 7.57 (br t, J=7.7 Hz, 2H), 7.31 (br d, J=9.0 Hz, 1H), 7.08 (d, J=2.0 Hz, 1H), 6.87 (dd, J=8.9, 2.3 Hz, 1H), 6.79 (s, 1H), 3.80 (s, 5H), 3.72-3.53 (m, 5H), 3.49-3.38 (m, 2H), 3.08 (br d, J=12.6 Hz, 2H), 2.19 (br s, 1H), 2.02 (br d, J=14.3 Hz, 3H), 1.70 (br s, 2H), 1.43-1.25 (m, 5H).

LCMS (ESI+): m/z 434.3 (M+H)

### Example 24: General Protocol U for Synthesis of Exemplary Compounds

General Protocol U to synthesize exemplary compounds of Formula (I) is described in Scheme 21 and the procedures set forth below.

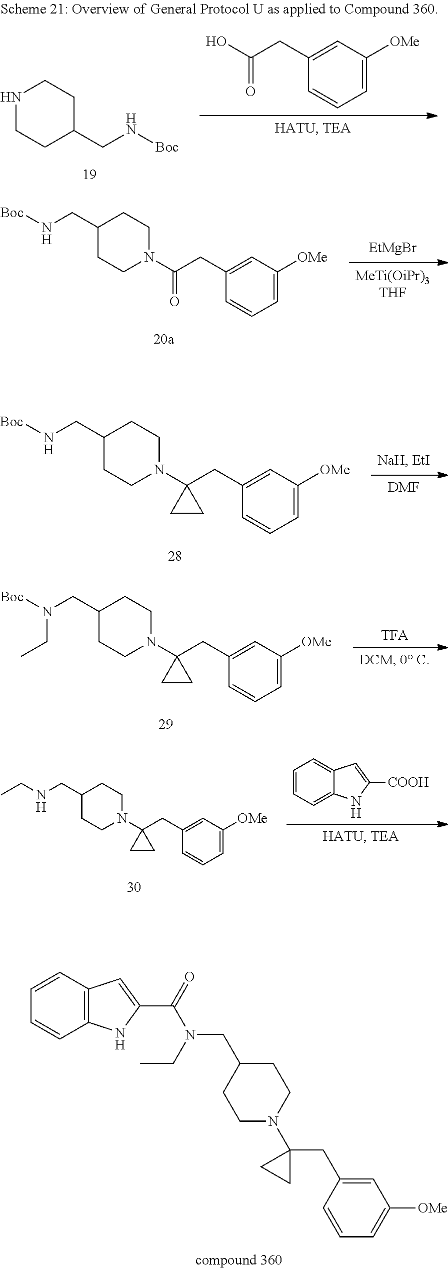

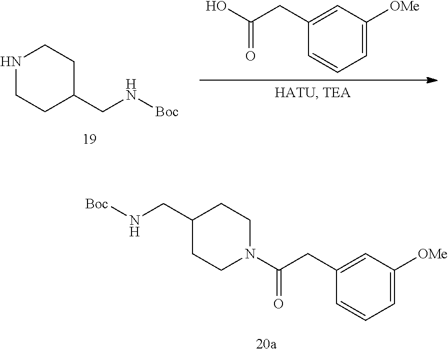

Procedure for the preparation of 20a: A mixture of compound 19 (1.5 g, 7.0 mmol, 1.0 eq), 3-methoxyphenyl acetic acid (1.2 g, 7.0 mmol, 1.0 eq), HATU (2.7 g, 7.0 mmol, 1.0 eq), TEA (1.4 g, 14.0 mmol, 1.9 mL, 2.0 eq) in 15 mL of DMF was stirred at 25° C. for 1 hour. The reaction was monitored by TLC and allowed to run until complete. The reaction mixture was diluted with 50 mL of ethyl acetate and washed twice with 100 mL of water. The organic layer was washed five times with 150 mL of brine, dried over Na₂SO₄, filtered and concentrated under reduced pressure to give 3.0 g of compound 20a as a yellow oil.

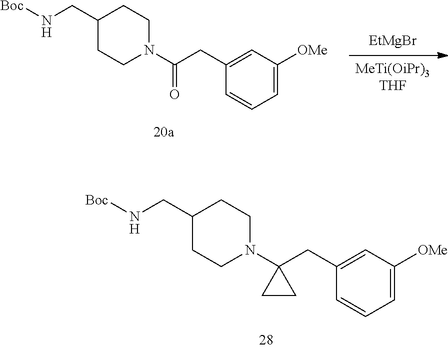 [SMILES: "EtMgBr" ; "MeTi(OiPr)3THF" ; COc1cccc(CC(=O)N2CCC(CNC(=O)OC(C)(C)C)CC2)c1 ; COc1cccc(CC2(N3CCC(CNC(=O)OC(C)(C)C)CC3)CC2)c1]

Procedure for the preparation of compound 28: A mixture of compound 20a (400 mg, 1.1 mmol, 1.0 eq) in 5 mL of THF was cooled to 0° C., triisopropoxy(methyl)titanium (636 mg, 2.7 mmol, 2.4 eq) was added in one portion and stirred for 15 min at 0° C., then EtMgBr (3M, 1.5 mL, 4.0 eq) was added dropwise. The mixture was stirred at 25° C. for another 1 hour under N₂ atmosphere. The reaction was monitored by TLC and allowed to run until complete. It was quenched by adding 20 mL of water, filtered to remove the solid, the filtrate was extracted with two 20 mL portions of ethyl acetate, the organic layer was washed with 25 mL of brine, dried over Na₂SO₄, filtered and evaporated under reduced pressure to give the crude product. The product was purified by prep-TLC (petroleum ether/ethyl acetate=2/1) to give 120 mg (29%) of compound 28 as a colorless gum.

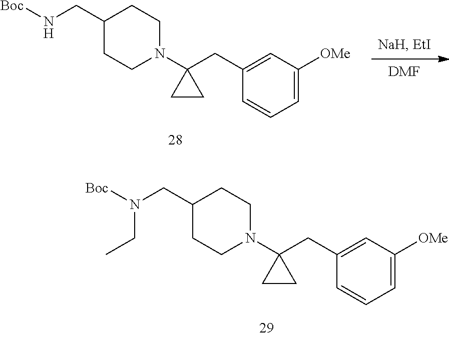

Procedure for the preparation of compound 29: A mixture of compound 28 (120 mg, 320.4 μmol, 1.0 eq) in 3 mL of DMF was cooled to 0° C. NaH (102.5 mg, 2.6 mmol, 60% purity, 8.0 eq) was added and stirred at 25° C. for 0.5 hour, then EtI (400 mg, 2.6 mmol, 205.0 μL, 8.0 eq) was added and the mixture was stirred at 25° C. for another 1.5 hours. The reaction was monitored by TLC and allowed to run until completion. The reaction mixture was partitioned between 15 mL of saturated aqueous NH₄Cl and 15 mL of ethyl acetate. The organic phase was separated, washed three times with 10 mL of water and 15 mL of brine, dried over Na₂SO₄, filtered and concentrated under reduced pressure to give 130 mg of the crude product compound 29 as yellow gum. This material was used in subsequent reactions without further purification.

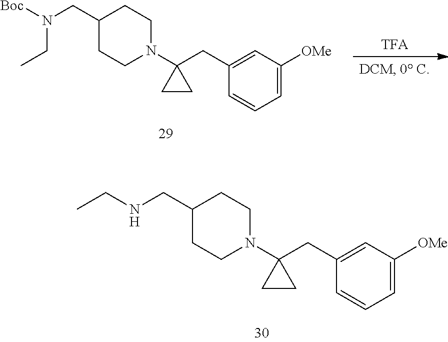 [SMILES: CCN(CC1CCN(C2(Cc3cccc(OC)c3)CC2)CC1)C(=O)OC(C)(C)C ; "DCM, 0° C." ; "TFA" ; CCNCC1CCN(C2(Cc3cccc(OC)c3)CC2)CC1]

Procedure for the preparation of compound 30: A mixture of compound 29 (50.0 mg, 124.2 μmol, 1.0 eq), TFA (1.2 g, 10.1 mmol, 750.0 μL, 81.6 eq) in 3 mL of DCM was stirred at 0° C. for 0.5 hour. The reaction was monitored by LCMS and allowed to run until complete. It was evaporated under reduced pressure (below 30° C.) to give the crude product compound 30 (55.0 mg, crude, TFA salt) as brown oil and to be used into the next step without further purification.

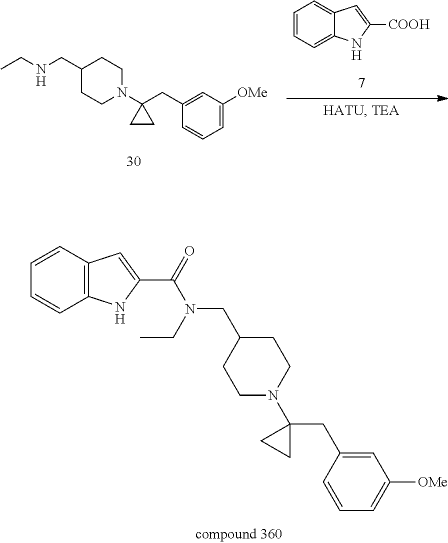 [SMILES: O=C(O)c1cc2ccccc2[nH]1 ; CCNCC1CCN(C2(Cc3cccc(OC)c3)CC2)CC1 ; "HATU, TEA" ; CCN(CC1CCN(C2(Cc3cccc(OC)c3)CC2)CC1)C(=O)c1cc2ccccc2[nH]1]

Procedure for the preparation of compound 360: A mixture of 1H-indole-2-carboxylic acid (20.0 mg, 124.1 μmol, 1.0 eq), compound 30 (51.7 mg, 124.1 μmol, 1.0 eq, TFA salt), TEA (37.7 mg, 372.3 μmol, 3.0 eq), and HATU (47.2 mg, 124.1 μmol, 1.0 eq) in 2 mL DMF was degassed and purged with N₂ 3 times. The mixture was stirred at 25° C. for 12 hours under N₂ atmosphere. The reaction was monitored by LCMS and allowed to run until completion. It was filtered and the filtrate was concentrated and purified by prep-HPLC (TFA condition) to give 49.9 mg (71%, TFA salt) of compound 360 as a brown solid.

¹H NMR (400 MHz, METHANOL-d₄) δ ppm 7.60 (br d, J=7.94 Hz, 1H) 7.42 (br d, J=8.16 Hz, 1H) 7.16-7.26 (m, 2H) 7.01-7.08 (m, 1H) 6.75-6.88 (m, 4H) 3.76 (s, 5H) 3.54 (br d, J=11.03 Hz, 4H) 3.34-3.44 (m, 2H) 3.16-3.23 (m, 1H) 3.20 (s, 1H) 2.13 (br s, 1H) 2.00 (br d, J=14.77 Hz, 2H) 1.41-1.63 (m, 1H) 1.53 (br d, J=9.70 Hz, 1H) 1.30 (br t, J=6.95 Hz, 3H) 1.03 (br s, 2H) 0.77 (br s, 2H)

LCMS (ESI+): m/z 446.1 (M+H)

### Example 25: General Protocol V for Synthesis of Exemplary Compounds

General Protocol V to synthesize exemplary compounds of Formula (I) is described in Scheme 22 and the procedures set forth below.

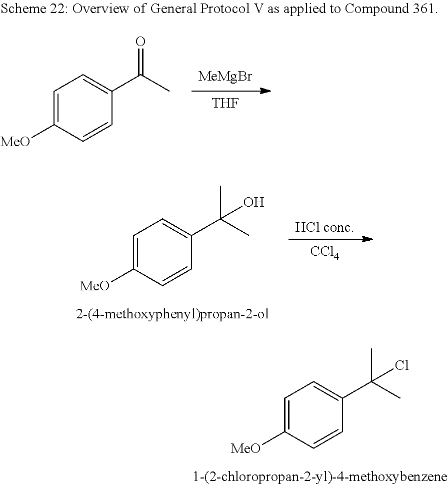

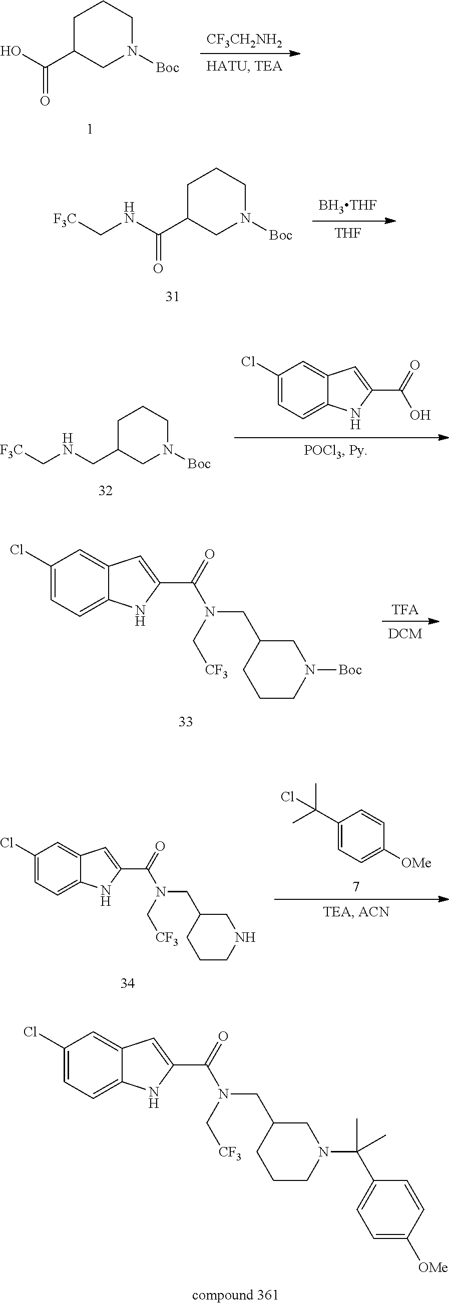

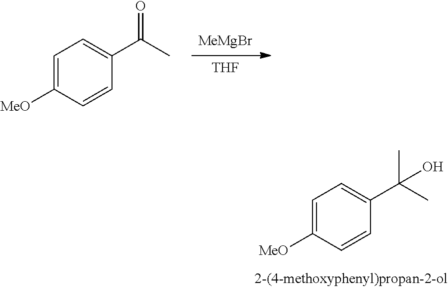

Procedure for the preparation of 2-(4-methoxyphenyl)propan-2-ol: To a solution of 4-methoxyacetophenone (5.0 g, 33.3 mmol, 1.0 eq) in 60 mL THF was added MeMgBr (3M, 33.3 mL, 3.0 eq) at 0° C. and the reaction was stirred for 12 hours at 20° C. The reaction was monitored by TLC and allowed to run until complete. The reaction mixture was quenched by 15 mL of saturated aqueous NH₄Cl and extracted with three 10 mL portions of ethyl acetate. The combined organic layers were washed twice with 20 mL of brine, dried over Na₂SO₄, filtered and the filtrate was concentrated to give a residue. The residue was purified by column chromatography (SiO₂, eluting with petroleum ether/ethyl acetate=100/1 to 10/1) to give 2.4 g of 2-(4-methoxyphenyl)propan-2-ol (43% yield) as an colorless oil.

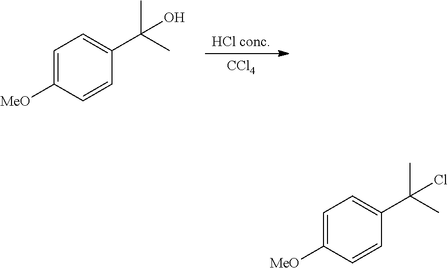

Procedure for the preparation of 1-(2-chloropropan-2-yl)-4-methoxybenzene: To a solution of 2-(4-methoxyphenyl)propan-2-ol (400 mg, 2.4 mmol, 1.0 eq) in 3 mL of CCl₄ was added 1 mL of 12N HCl (5.0 eq) at 0° C. and the reaction was stirred for 15 mins at this temperature. The reaction was monitored by TLC and allowed to run until complete. The reaction mixture was separated to isolate the CCl₄ layer and the alkyl chloride was used crude as a pink solution in CCl₄.

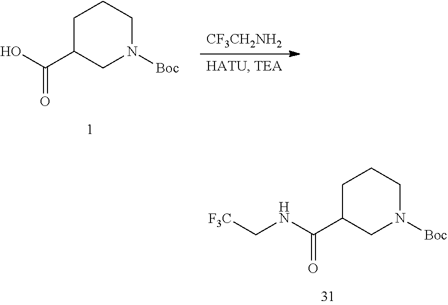 [SMILES: CC(C)(C)OC(=O)N1CCCC(C(=O)O)C1 ; "CF3CH2NH2" ; "HATU, TEA" ; CC(C)(C)OC(=O)N1CCCC(C(=O)NCC(F)(F)F)C1]

Procedure for the preparation of compound 31: To a mixture of compound 1 (15.0 g, 65.4 mmol, 1.1 eq) in 150 mL of DMF was added HATU (24.9 g, 65.4 mmol, 1.1 eq) and Et₃N (18.1 g, 178.5 mmol, 3.0 eq) in one portion at 25° C. The mixture was stirred at 25° C. for 0.5 hour. Then 2,2,2-trifluoroethanamine (5.9 g, 59.5 mmol, 1.0 eq) was added. The reaction mixture was stirred at 25° C. for 4 hours. The reaction was monitored by LCMS and allowed to run until complete. The mixture was poured into 300 mL of ice-water and extracted with three 150 mL portions of ethyl acetate. The combined organic phase was washed twice with 200 mL of brine, dried with anhydrous Na₂SO₄, filtered and concentrated in vacuum to give compound 31 (12.0 g, 38.7 mmol, 65.0% yield) as a colorless oil.

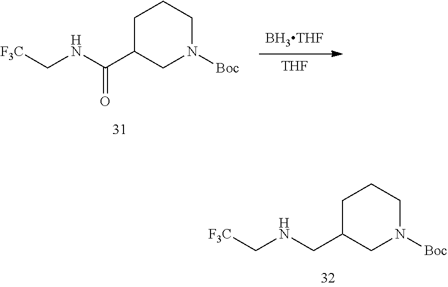 [SMILES: CC(C)(C)OC(=O)N1CCCC(C(=O)NCC(F)(F)F)C1 ; CC(C)(C)OC(=O)N1CCCC(CNCC(F)(F)F)C1 ; "BH3·THF" ; "THF"]

Procedure for the preparation of compound 32: To a mixture of compound 31 (12.0 g, 38.7 mmol, 1.0 eq) in 150 mL of THF was added BH₃.THF (1M, 116.0 mL, 3.0 eq) at 25° C., and then the mixture was stirred at 70° C. for 16 hours under N₂ atmosphere. The reaction was monitored by LCMS and allowed to run until complete. The mixture was cooled in a water bath, and quenched with 200 mL of MeOH, then the mixture was stirred at 70° C. for 1 hour, then concentrated to give 10.6 g amine 32 as a colorless oil.

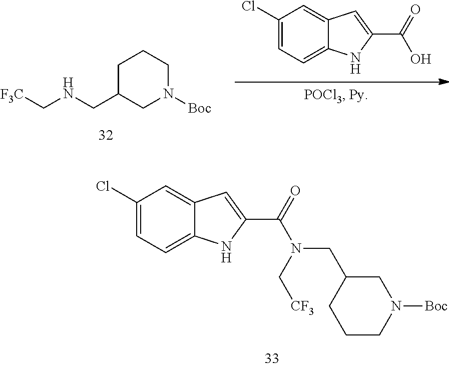

Procedure for the preparation of Compound 33: To a mixture of amine 32 (250.0 mg, 843.7 μmol, 1.0 eq) and 5-chloro-indol-2-carboxylic acid (165.0 mg, 843.7 μmol, 1.0 eq) in 5 mL of pyridine was added POCl₃ (388.1 mg, 2.5 mmol, 3.0 eq) dropwise at 0° C. The mixture was stirred at 25° C. for 1 hour. The reaction was monitored by TLC and allowed to run until complete. The reaction was quenched by saturated aqueous NaHCO₃ and the pH adjusted to pH 7. The mixture was concentrated in vacuum. The residue was dissolved in 10 mL of H₂O, and extracted with three 10 mL portions of ethyl acetate. The combined organic phase was washed with 20 mL of brine, dried with anhydrous Na₂SO₄, filtered and concentrated in vacuum. The residue was purified by prep-TLC (SiO₂, eluting with petroleum ether/ethyl acetate=3/1) to give 160 mg (20%) of compound 33 as a yellow oil. This material was used directly in the next reaction.

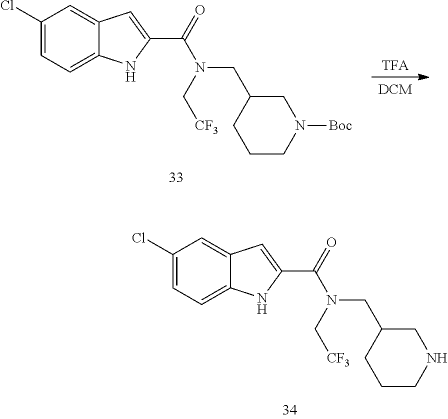

Procedure for the preparation of compound 34: A mixture of compound 33 (160.0 mg, 337.6 μmol, 1.0 eq) in 1 mL of DCM and TFA (308.0 mg, 2.7 mmol, 8.0 eq) was stirred at 25° C. for 2 hours. The reaction was monitored by TLC and allowed to run until complete. The reaction mixture was adjusted to pH˜8 by saturated NaHCO₃, and extracted with three 3 mL portions of DCM. The combined organic phase was dried with anhydrous Na₂SO₄, filtered and concentrated in vacuum to give 100 mg (79%) of compound 34 as a yellow solid.

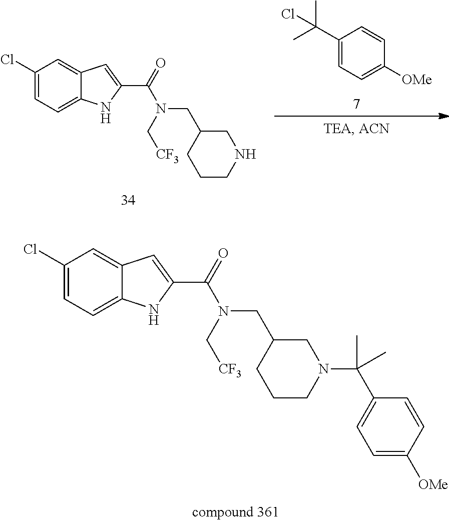

Procedure for the preparation of compound 361: To a solution of compound 34 (24.0 mg, 64.2 μmol, 1.0 eq) and TEA (1.5 g, 14.4 mmol, 224.7 eq) in 3 mL of ACN was added a solution of 1-(2-chloropropan-2-yl)-4-methoxybenzene (400 mg, 2.2 mmol, 33.7 eq) in 3 mL of CCl₄ at 0° C. The reaction was stirred for 1 hour at 20° C. The reaction was monitored by LCMS and allowed to run until complete. The reaction mixture was concentrated to give a residue. The reaction mixture was purified by prep-TLC and then repurified by prep-HPLC (TFA condition) to give 3.7 mg (9%) of the TFA salt of compound 361 as a white solid.

¹H NMR (400 MHz, DMSO-d₆) δ (400 MHz, DMSO-d₆) δ ppm 11.90 (br s, 1H), 7.67 (s, 1H), 7.41-7.51 (m, 3H), 7.19-7.25 (m, 1H), 6.89-6.98 (m, 2H), 6.80 (br s, 1H), 4.45 (br s, 2H), 3.71 (s, 3H), 3.59 (br s, 2H), 3.10-3.26 (m, 1H), 2.25 (br s, 2H), 1.57-1.84 (m, 9H), 1.21 (br s, 1H), 1.06 (br s, 2H)

LCMS (ESI+): m/z 522.2 (M+H)

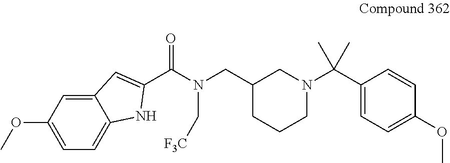

¹H NMR (400 MHz, DMSO-d₆) δ (400 MHz, DMSO-d₆) δ ppm 11.57 (br s, 1H), 7.50 (br d, J=8.4 Hz, 2H), 7.36 (d, J=8.9 Hz, 1H), 7.09 (d, J=2.1 Hz, 1H), 6.95 (br d, J=8.7 Hz, 2H), 6.91 (dd, J=8.9, 2.3 Hz, 1H), 6.76 (br s, 1H), 4.48 (br d, J=8.1 Hz, 2H), 3.78 (s, 3H), 3.74 (s, 3H), 3.55-3.69 (m, 3H), 3.17-3.28 (m, 1H), 2.28 (br s, 1H), 1.56-1.87 (m, 10H), 1.17-1.28 (m, 1H), 1.02-1.16 (m, 1H)

LCMS (ESI+): m/z 518.3 (M+H)

### Example 26: General Protocol W for Synthesis of Exemplary Compounds

General Protocol W to synthesize exemplary compounds of Formula (I) is described in Scheme 23 and the procedures set forth below.

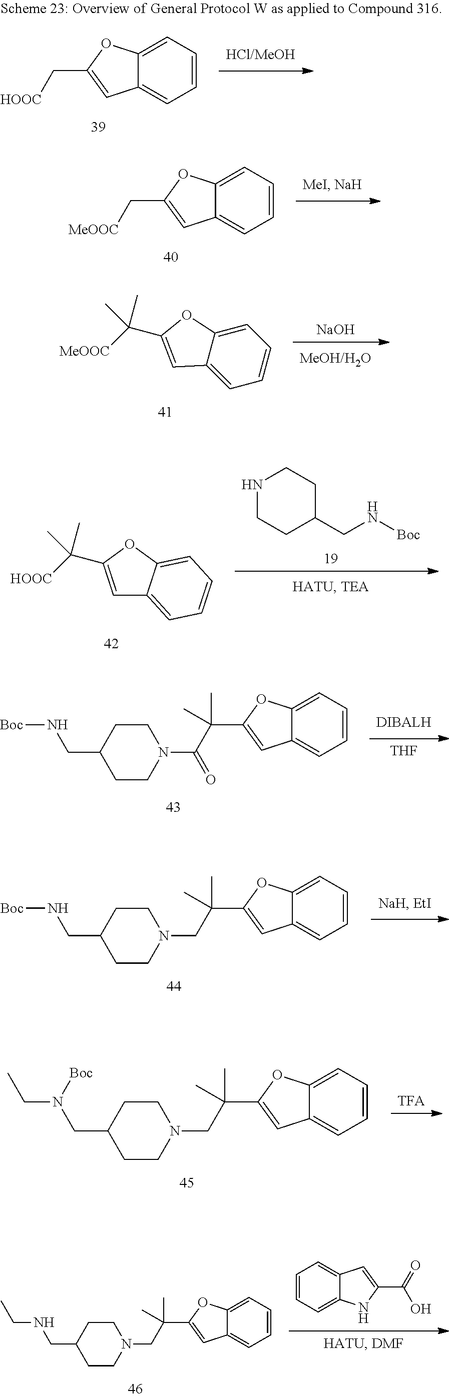

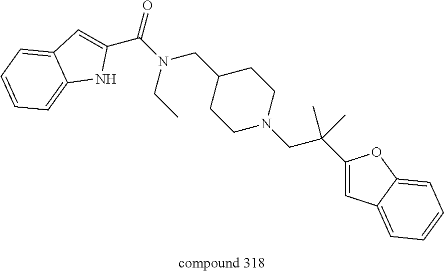 [SMILES: CCN(CC1CCN(CC(C)(C)c2cc3ccccc3o2)CC1)C(=O)c1cc2ccccc2[nH]1]

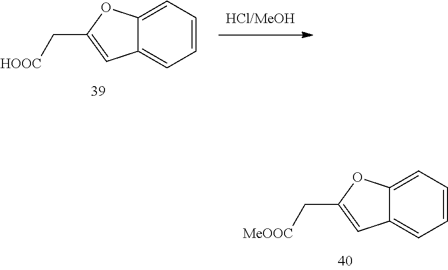 [SMILES: O=C(O)Cc1cc2ccccc2o1 ; COC(=O)Cc1cc2ccccc2o1]

General procedure for the preparation of ester 40: A solution of compound 39 (800 mg, 4.5 μmol) in HCl/MeOH (4M, 10.0 mL) was stirred for 2 h at 70° C. The reaction was monitored by TLC and allowed to run until complete. The reaction mixture was concentrated and the residue was diluted with 10 mL of ethyl acetate and washed twice with 10 mL of water, then 10 mL of brine and concentrated to give 1.0 g of ester 40 as a brown oil.

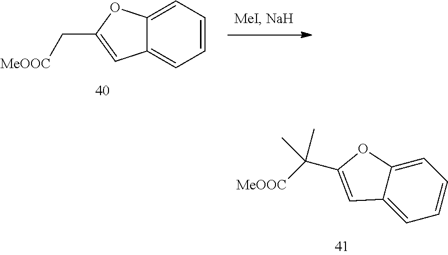 [SMILES: COC(=O)Cc1cc2ccccc2o1 ; COC(=O)C(C)(C)c1cc2ccccc2o1]

General procedure for the preparation of compound 41: To a solution of methyl ester 40 (800 mg, 4.2 μmol, 1.0 eq) in 2.0 mL of was added NaH (404 mg, 10.1 μmol, 60% purity, 2.4 eq) at 0° C. After stirring for 30 min, MeI (1.8 g, 12.6 μmol, 3.0 eq) was added into the mixture at 0° C. and the reaction was stirred for another 30 min at this temperature. The reaction was monitored by TLC and allowed to run until complete. The reaction mixture was quenched with iced aqueous NH₄Cl (10 mL) and extracted with three 10 mL portions of ethyl acetate. The combined organic layers were washed twice with 20 mL of brine, dried over Na₂SO₄, filtered and the filtrate was concentrated. The residue was purified by prep-TLC (SiO₂, eluting with petroleum ether/ethyl acetate=5/1) to give 390 mg of methyl ester 41 as a yellow oil.

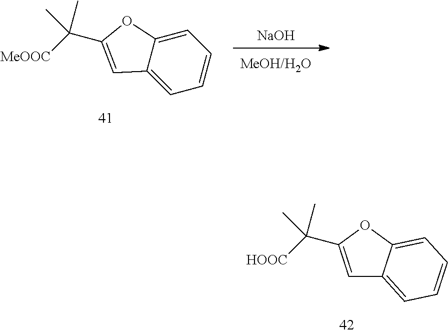 [SMILES: COC(=O)C(C)(C)c1cc2ccccc2o1 ; "NaOH" ; CC(C)(C(=O)O)c1cc2ccccc2o1]

General procedure for the preparation of compound 42: A mixture of methyl ester 41 (200 mg, 916 μmol, 1.0 eq) and NaOH (183 mg, 4.6 μmol, 5.0 eq) in 5.0 mL of methanol and 2.0 mL of water was stirred for 2 h at 20° C. The reaction was monitored by TLC and allowed to run until complete. The reaction mixture was concentrated to remove methanol. The remaining solution was made acidic with 1N HCl to pH 2-3. Then the mixture was extracted with three 5 mL portions of ethyl acetate. The combined organic layers were dried over Na₂SO₄, filtered and the filtrate was concentrated to give 180 mg of compound 42 as a yellow solid.

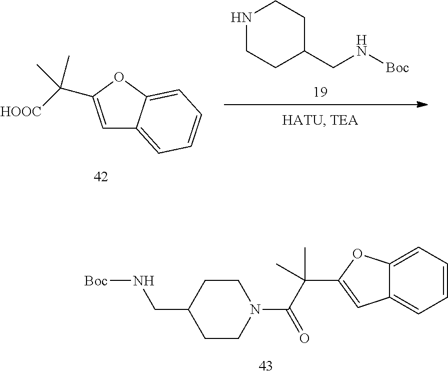

General procedure for the preparation of compound 43: To a solution of compound 42 (89.1 mg, 436.3 μmol, 1.1 eq) and TEA (200.7 mg, 2.0 μmol, 5.0 eq) in 2.0 mL of DMF was added HATU (166 mg, 436 μmol, 1.1 eq) at 20° C. After stirring for 30 min, amine 19 (85.0 mg, 396.6 μmol, 1.0 eq) was added into the mixture and the final reaction was stirred for 16 h at 20° C. The reaction was monitored by LCMS and allowed to run until complete. The reaction mixture was diluted with 10 mL of water and extracted with three 10 mL portions of ethyl acetate. The combined organic layers were washed twice with 20 mL of brine, dried over Na₂SO₄, filtered and the filtrate was concentrated. The residue was purified by prep-TLC (SiO₂, eluting with petroleum ether/ethyl acetate=2/1) to give 185 mg of compound 43 as a light yellow solid.

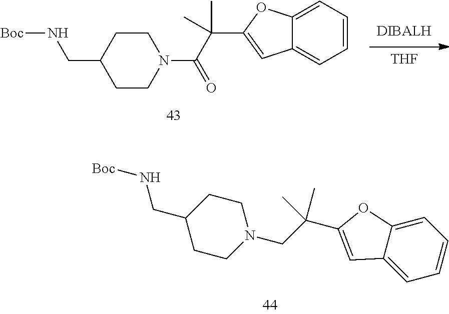 [SMILES: CC(C)(C)OC(=O)NCC1CCN(C(=O)C(C)(C)c2cc3ccccc3o2)CC1 ; "THF" ; CC(C)(C)OC(=O)NCC1CCN(CC(C)(C)c2cc3ccccc3o2)CC1]

General procedure for the preparation of compound 44: To a solution of compound 43 (80.0 mg, 199.8 μmol, 1.0 eq) in 1.5 mL of THF was added DIBAL-H (1M, 1.0 mL, 5.0 eq) at 0° C. The reaction was stirred at 0° C. for an hour then 50° C. for 12 h. The reaction was monitored by LCMS and allowed to run until complete. The reaction mixture was poured into 10 mL of aqueous Na₂CO₃ and extracted with three 10 mL portions of ethyl acetate. The combined organic layers were washed twice with 20 mL of brine, dried over Na₂SO₄, filtered and the filtrate was concentrated to give 75 mg of compound 44 as a colorless gum.

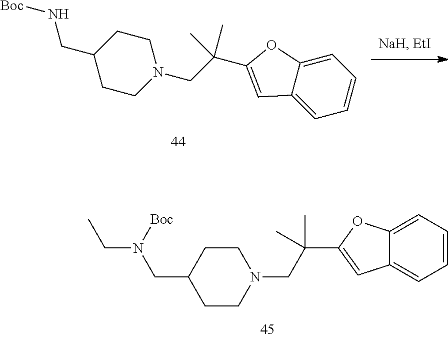 [SMILES: CC(C)(C)OC(=O)NCC1CCN(CC(C)(C)c2cc3ccccc3o2)CC1 ; CCN(CC1CCN(CC(C)(C)c2cc3ccccc3o2)CC1)C(=O)OC(C)(C)C]

General procedure for the preparation of compound 45: To a solution of compound 44 (75.0 mg, 194.0 μmol, 1.0 eq) in 2.0 mL of DMF was added NaH (15.5 mg, 388.1 μmol, 60% purity, 2.0 eq) at 0° C. After stirring for 15 min at 0° C., EtI (60.5 mg, 388.1 μmol, 2.0 eq) was added and the reaction was allowed to warm to 20° C. and stirred for 45 min at this temperature. The reaction was monitored by TLC and allowed to run until complete. The reaction mixture was quenched with 10 mL of iced saturated aqueous NH₄Cl and extracted three times with 10 mL of ethyl acetate. The combined organic layers were washed twice with 20 mL of brine, dried over Na₂SO₄, filtered and the filtrate was concentrated to give 80 mg of compound 45 as a yellow gum.

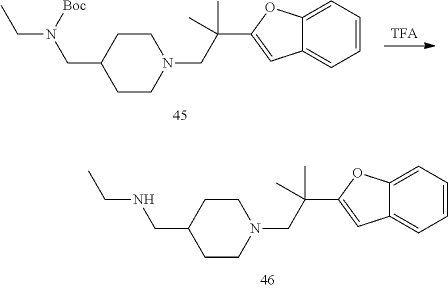 [SMILES: "TFA" ; CCN(CC1CCN(CC(C)(C)c2cc3ccccc3o2)CC1)C(=O)OC(C)(C)C ; CCNCC1CCN(CC(C)(C)c2cc3ccccc3o2)CC1]

General procedure for the preparation of compound 46: A solution of compound 45 (80.0 mg, 193.0 μmol, 1.0 eq) in 2.0 mL of DCM containing TFA (500.0 μL) was stirred for 1 h at 20° C. The reaction was monitored by TLC and allowed to run until completion. The reaction was concentrated to give 85 mg of crude compound 46 (TFA salt) as a brown gum.

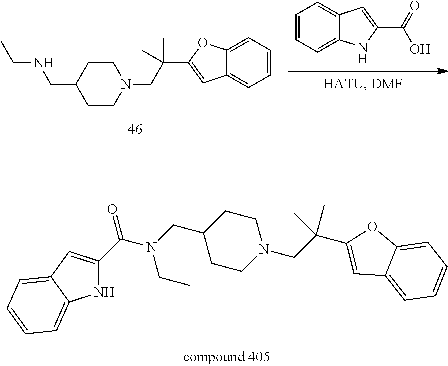

General procedure for the preparation of compound 405: To a solution of indole-2-carboxylic acid (35 mg, 218 μmol, 1.1 eq) and TEA (100 mg, 992 μmol, 5.0 eq) in 1.5 mL of DMF was added HATU (83.0 mg, 218 μmol, 1.1 eq) at 20° C. After stirring for 30 min, compound 46 (85.0 mg, 198.4 μmol, 1.0 eq, TFA salt) was added and the reaction was stirred for 12 h at 20° C. The reaction was monitored by LCMS and allowed to run until complete. The reaction mixture was filtered and the solution was purified by prep-HPLC (TFA condition) to give 14.2 mg of compound 405 (TFA salt) as a white solid.

¹H NMR (400 MHz, METHANOL-d₄) 6=ppm 7.62-7.52 (m, 2H), 7.45 (br d, J=8.8 Hz, 1H), 7.38 (br d, J=8.2 Hz, 1H), 7.30-7.15 (m, 3H), 7.07-7.00 (m, 1H), 6.84-6.71 (m, 2H), 3.66 (br d, J=17.9 Hz, 1H), 3.54 (br s, 4H), 3.25-2.97 (m, 3H), 2.96-2.88 (m, 1H), 2.15-1.90 (m, 2H), 1.78 (br s, 2H), 1.63-1.34 (m, 6H), 1.32-1.15 (m, 4H).

LCMS (ESI+): m/z 458.1 (M+H)

The following compounds were prepared analogously:

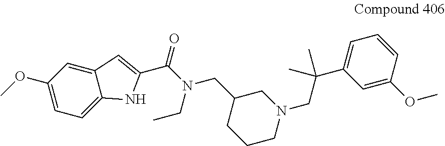

¹H NMR (400 MHz, METHANOL-d₄) δ ppm 7.36 (br d, J=8.8 Hz, 1H), 7.18-7.10 (m, 2H), 7.03-6.89 (m, 3H), 6.75 (br s, 1H), 6.63 (br s, 1H), 3.82 (s, 3H), 3.65 (s, 3H), 3.63-3.37 (m, 4H), 3.29-3.17 (m, 3H), 2.99 (br d, J=13.2 Hz, 1H), 2.82 (br s, 1H), 2.57 (br s, 1H), 2.28 (br s, 1H), 1.82 (br s, 3H), 1.47 (s, 3H), 1.40 (s, 3H), 1.33 (br s, 1H), 1.23 (br t, J=6.6 Hz, 1H), 1.18 (br s, 1H).

LCMS (ESI+): m/z 478.2 (M+H)

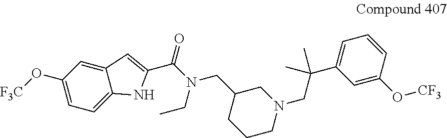 [SMILES: CCN(CC1CCCN(CC(C)(C)c2cccc(OC(F)(F)F)c2)C1)C(=O)c1cc2cc(OC(F)(F)F)ccc2[nH]1]

¹H NMR (400 MHz, METHANOL-d₄) δ ppm 7.57 (br s, 1H), 7.54-7.45 (m, 2H), 7.43-7.29 (m, 2H), 7.16 (br d, J=8.8 Hz, 1H), 7.05 (br s, 1H), 6.98-6.79 (m, 1H), 3.74-3.33 (m, 6H), 3.28-3.05 (m, 2H), 2.92-2.59 (m, 2H), 2.37 (br d, J=7.9 Hz, 1H), 1.83 (br s, 3H), 1.49 (br d, J=10.4 Hz, 6H), 1.33-1.19 (m, 4H)

LCMS (ESI+): m/z 586.3 (M+H)

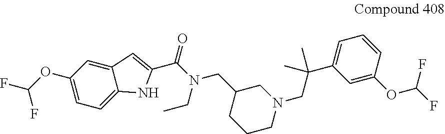 [SMILES: CCN(CC1CCCN(CC(C)(C)c2cccc(OC(F)F)c2)C1)C(=O)c1cc2cc(OC(F)F)ccc2[nH]1]

¹H NMR (400 MHz, METHANOL-d₄) δ ppm 7.50-7.43 (m, 2H), 7.32-7.21 (m, 3H), 7.08 (br d, J=8.8 Hz, 1H), 7.00-6.52 (m, 4H), 3.67-3.38 (m, 3H), 3.11-2.97 (m, 2H), 2.91-2.77 (m, 2H), 2.42-2.26 (m, 2H), 1.89-1.73 (m, 4H), 1.57-1.41 (m, 6H), 1.37-1.19 (m, 5H).

LCMS (ESI+): m/z 550.3 (M+H)

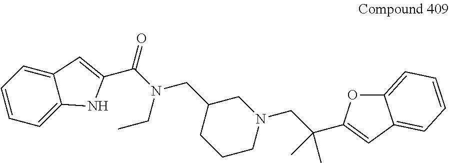 [SMILES: CCN(CC1CCCN(CC(C)(C)c2cc3ccccc3o2)C1)C(=O)c1cc2ccccc2[nH]1]

¹H NMR (methanol-d₄, 400 MHz) δ ppm 7.67 (br d, J=7.9 Hz, 1H), 7.48 (br d, J=7.9 Hz, 1H), 7.40-7.23 (m, 3H), 7.15-7.04 (m, 3H), 6.73-6.58 (m, 2H), 3.66-3.49 (m, 2H), 3.46-3.32 (m, 2H), 3.17-3.06 (m, 2H), 2.94 (br s, 2H), 2.75 (br t, J=12.6 Hz, 1H), 2.39 (br s, 1H), 1.92-1.77 (m, 3H), 1.56 (s, 3H), 1.50 (s, 3H), 1.36-1.19 (m, 2H), 1.11 (br t, J=6.9 Hz, 3H).

LCMS (ESI+): m/z 458.2 (M+H)

### Example 26: General Protocol X for Synthesis of Exemplary Compounds

General Protocol X to synthesize exemplary compounds of Formula (I) is described in Scheme 24 and the procedures set forth below.

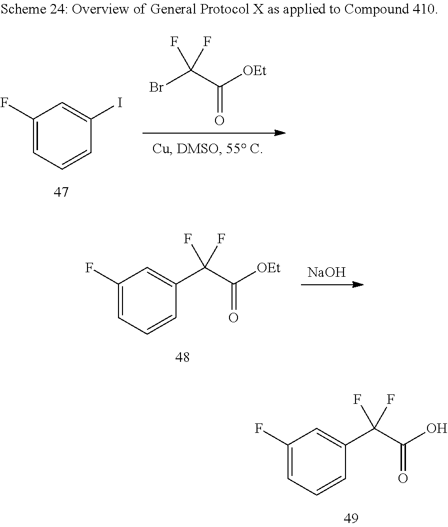

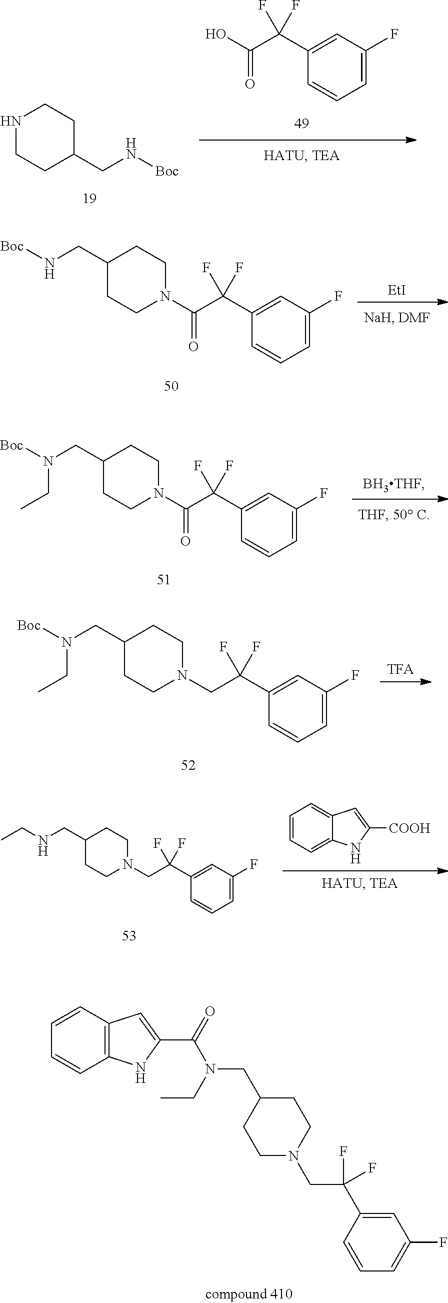

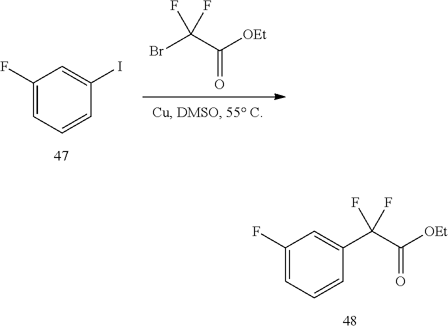

General procedure for the preparation of ethyl ester 48: To a solution of compound 47 (3.0 g, 13.5 μmol, 1.0 eq) and ethyl 2-bromo-2,2-difluoro-acetate (5.5 g, 27.0 μmol, 2.0 eq) in 30 mL of DMSO was added Cu powder (2.6 g, 40.5 μmol, 3.0 eq). The mixture was stirred at 60° C. for 12 hours under N₂. The reaction was monitored by TLC and allowed to run until complete. The reaction mixture was quenched by adding 40 mL of water and then extracted with three 15 mL portions of ethyl acetate. The combined organic layers were washed twice with 20 mL portions of brine, dried over Na₂SO₄, filtered and concentrated under reduced pressure to give a residue. The residue was purified by column chromatography (SiO2, eluting with petroleum ether/ethyl acetate=100/1 to 20/1) to give 1.8 g ethyl ester 48 as white solid.

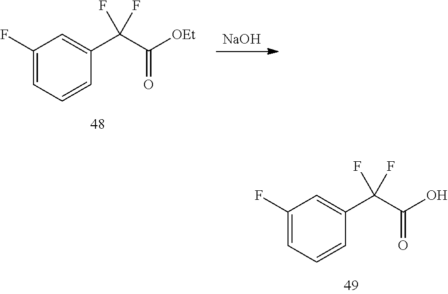 [SMILES: CCOC(=O)C(F)(F)c1cccc(F)c1 ; "NaOH" ; O=C(O)C(F)(F)c1cccc(F)c1]

General procedure for the preparation of carboxylic acid 49: To a solution of ethyl ester 48 (1.6 g, 7.3 μmol, 1.0 eq) in 15.0 mL of MeOH and 5 mL of water was added NaOH (881 mg, 20.0 μmol, 3.0 eq). The mixture was stirred at 25° C. for 1 hour. The reaction was monitored by TLC and allowed to run until complete. The mixture combined with another similar batch was concentrated to remove MeOH, then made acidic with 1N HCl to pH˜2. The aqueous solution was extracted with three 10 mL portions of DCM and the combined organic layers were dried over Na₂SO₄ and concentrated to give a 2.5 g of crude acid 49 as a white solid.

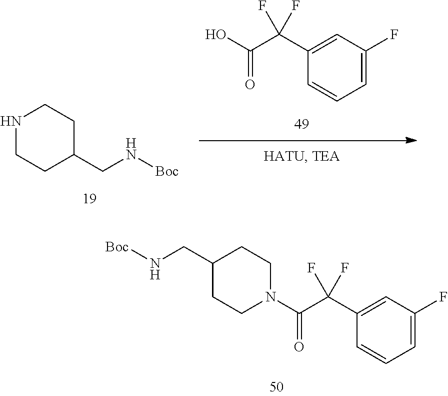 [SMILES: O=C(O)C(F)(F)c1cccc(F)c1 ; CC(C)(C)OC(=O)NCC1CCNCC1 ; CC(C)(C)OC(=O)NCC1CCN(C(=O)C(F)(F)c2cccc(F)c2)CC1]

General procedure for the preparation of compound 50: To a solution of compound 19 (200 mg, 933 μmol, 1.0 eq) and compound 49 (177 mg, 933 μmol, 1.00 eq) in 8 mL of DMF was added HATU (355 mg, 933 μmol, 1.0 eq) and TEA (189 mg, 1.9 μmol, 2.0 eq). The mixture was stirred at 25° C. for 1 hour. The reaction was monitored by TLC and allowed to run until complete. The reaction mixture was quenched by addition of 20 mL of aqueous NH₄Cl and then extracted with three 5 mL portions of ethyl acetate. The combined organic layers were washed twice with 25 mL portions of brine, dried over Na₂SO₄, filtered and concentrated under reduced pressure to give 265 mg of the crude product compound 50 as a yellow oil, which was used to do next step without further purification.

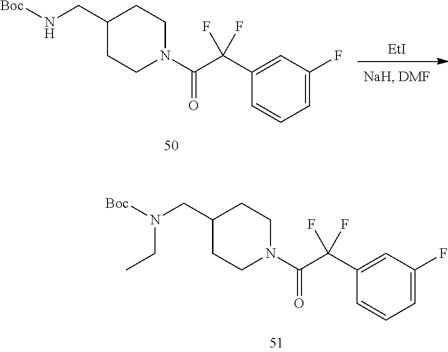 [SMILES: "EtI" ; CC(C)(C)OC(=O)NCC1CCN(C(=O)C(F)(F)c2cccc(F)c2)CC1 ; CCN(CC1CCN(C(=O)C(F)(F)c2cccc(F)c2)CC1)C(=O)OC(C)(C)C]

General procedure for preparation of compound 51: To a solution of compound 50 (265 mg, 686 μmol, 1.0 eq) in 10 mL of DMF was added NaH (55 mg, 1.4 μmol, 60% purity, 2.0 eq) at 0° C. EtI (214 mg, 1.4 μmol, 2.0 eq) was added. The mixture was stirred at 25° C. or 3 hours. The reaction mixture was quenched by adding 20 mL of aqueous NH₄Cl and then extracted with three 8 mL portions of ethyl acetate. The combined organic layers were washed twice with 20 mL portions of brine, dried over Na₂SO₄, filtered and concentrated under reduced pressure to give a residue. The residue was purified by SiO₂ chromatography, eluting with petroleum ether/ethyl acetate=10/1 to 1:1) to give 90 mg of compound 51 as a yellow oil.

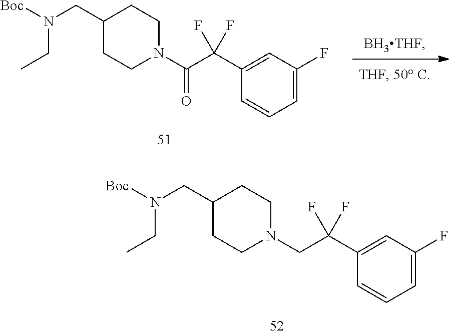 [SMILES: CCN(CC1CCN(C(=O)C(F)(F)c2cccc(F)c2)CC1)C(=O)OC(C)(C)C ; "BH3·THF," ; CCN(CC1CCN(CC(F)(F)c2cccc(F)c2)CC1)C(=O)OC(C)(C)C]

General procedure for the preparation of compound 52: To a solution of compound 53 (90.0 mg, 217.1 μmol, 1.0 eq) in 2 mL of THF was added BH₃.THF (1M, 652 μL, 3.0 eq) at 0° C. The mixture was stirred at 50° C. for 12 hours. The reaction mixture was quenched by addition of 5 mL of MeOH at 0° C. and concentrated under reduced pressure to give 90 mg of the amine 52 as a yellow oil, which was used to do next step without further purification.

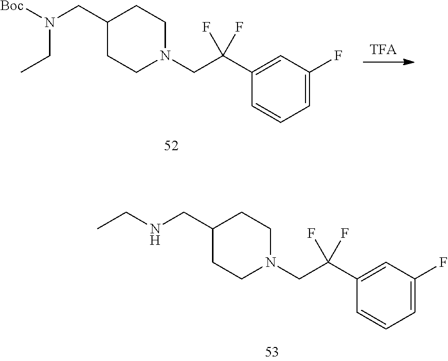 [SMILES: "TFA" ; CCN(CC1CCN(CC(F)(F)c2cccc(F)c2)CC1)C(=O)OC(C)(C)C ; CCNCC1CCN(CC(F)(F)c2cccc(F)c2)CC1]

General procedure for preparation of compound 53: To a solution of compound 52 (90 mg, 235 μmol, 1.0 eq) in 1 mL of DCM was added TFA (537 mg, 4.7 μmol, 20.0 eq). The mixture was stirred at 25° C. for 1 hour. The reaction was monitored by TLC and allowed to run until completion. The mixture was basified by 10% aqueous NaHCO₃ to pH˜8, and then extracted with three 5 mL potions of DCM. The combined organic layers were concentrated to give 60 mg of the crude amine 53 as a yellow oil. This material was used to do next step without further purification.

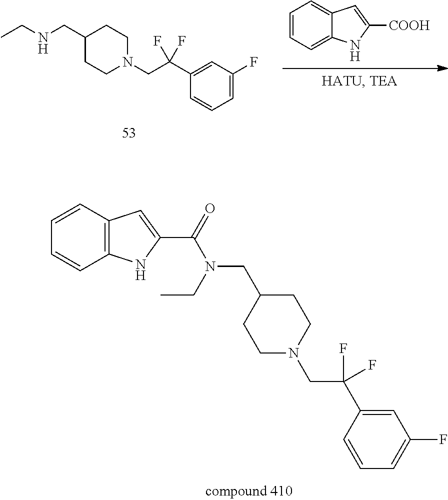

General procedure for the preparation of compound 410: To a solution of compound 53 (60 mg, 200 μmol, 1.0 eq) and 1H-indole-2-carboxylic acid (32.2 mg, 200 μmol, 1.0 eq) in 2 mL of DMF was added HATU (76 mg, 200 μmol, 1.00 eq) and TEA (61 mg, 600 μmol, 3.0 eq). The mixture was stirred at 25° C. for 12 hours. The reaction was monitored by TLC and allowed to run until complete. The reaction mixture was quenched by adding 10 mL of aqueous NH₄Cl, and then extracted with three 3 mL portions of ethyl acetate. The combined organic layers were washed with twice with 20 mL of brine, dried over Na₂SO₄, filtered and concentrated under reduced pressure to give a residue. The residue was purified by TLC (SiO2, eluting with petroleum ether/ethyl acetate=1/1) to give 44.1 mg of amide 410 as a white solid.

¹H NMR (400 MHz, CHLOROFORM-d) δ (400 MHz, CHLOROFORM-d) δ ppm 9.25 (br s, 1H) 7.64 (d, J=7.94 Hz, 1H) 7.30-7.42 (m, 2H) 7.27 (br d, J=4.63 Hz, 1H) 7.21 (br d, J=9.48 Hz, 1H) 7.04-7.15 (m, 2H) 6.78 (br s, 1H) 3.31-3.87 (m, 4H) 2.90 (t, J=14.11 Hz, 2H) 2.80 (br d, J=11.25 Hz, 2H) 2.22 (br t, J=11.36 Hz, 2H) 1.78 (br s, 1H) 1.67-1.71 (m, 1H) 1.62 (br s, 2H) 1.21-1.39 (m, 5H)

LCMS (ESI+): m/z 444.1 (M+H)

The following compounds were prepared analogously:

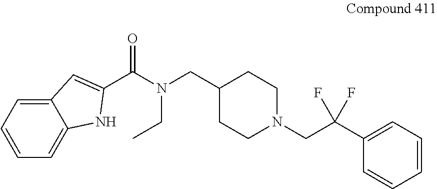 [SMILES: CCN(CC1CCN(CC(F)(F)c2ccccc2)CC1)C(=O)c1cc2ccccc2[nH]1]

¹H NMR (400 MHz, CHLOROFORM-d) δ ppm 9.27 (br s, 1H) 7.64 (d, J=7.94 Hz, 1H) 7.45-7.50 (m, 1H) 7.35-7.42 (m, 3H) 7.25-7.29 (m, 1H) 7.05-7.17 (m, 1H) 6.78 (br s, 1H) 3.30-3.91 (m, 4H) 2.92 (br t, J=14.33 Hz, 2H) 2.84 (br d, J=11.25 Hz, 2H) 2.22 (br t, J=11.25 Hz, 2H) 1.78 (br s, 1H) 1.59 (br s, 4H) 1.22-1.40 (m, 4H)

LCMS (ESI+): m/z 426.1 (M+H)

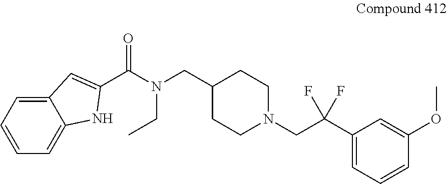 [SMILES: CCN(CC1CCN(CC(F)(F)c2cccc(OC)c2)CC1)C(=O)c1cc2ccccc2[nH]1]

¹H NMR (400 MHz, CHLOROFORM-d) δ ppm 9.29 (br s, 1H) 7.41 (d, J=8.33 Hz, 1H) 7.26-7.33 (m, 2H) 7.09-7.16 (m, 1H) 7.09-7.09 (m, 1H) 7.00-7.09 (m, 2H) 6.94 (br d, J=7.89 Hz, 1H) 6.80 (br s, 1H) 3.82 (s, 3H) 3.80-3.80 (m, 1H) 3.80-3.80 (m, 1H) 3.80-3.80 (m, 1H) 3.44 (br s, 4H) 2.83-2.99 (m, 4H) 2.25 (br t, J=11.18 Hz, 2H) 1.80 (br s, 1H) 1.59-1.69 (m, 4H) 1.33 (br d, J=9.65 Hz, 3H)

LCMS (ESI+): m/z 474.2 (M+H)

### Example 27. Dose Response Assay for TDP-43 Inhibition

Exemplary compounds of the invention were evaluated for efficacy in inhibiting TDP-43 inclusions using a dose response assay. Briefly, PC12 cells stably expressing wild type (WT) TDP-43-GFP were stressed with 15 μM to induce TDP-43 inclusions. The cells were then treated with exemplary compounds of the invention and the inhibitory effect on TDP-43 inclusions was observed using fluorescent microscopy. The ratio of cells with TDP-43 inclusions was calculated based on the total number of cells with detectable GFP expression. A 12-point dose response curve was generated, and the IC₅₀ for each compound tested was determined. Results of the dose response assay for exemplary compounds of the invention are summarized in Table 2, wherein A represents an IC₅₀ value of <100 nM; B represents an IC₅₀ value of 101-250 nM; C represents an IC₅₀ value of 251-500 nM; D represents an IC₅₀ value of >500 nM; and ND signifies that the IC₅₀ value was not determined.

### Example 28. Neuroprotection Assay

### Assay Media:

CMF dissection buffer: 1× Hank's balanced salt solution (Ca—/Mg, 500 mL) and 10 mM HEPES, pH 7.25-7.3 (1M stock, 5 mL)

Plating media: MEM (Earle salts+/Glutamine, 95 mL), FBS (to 2.5%, 2.5 mL), Pen/Strep (lx, 1 mL), glutamine (lx, 1 mL), and D-glucose (0.6% w/v, 0.6 g)

Feeding media: neurobasal media (96 mL), B27 supplement (2 mL), Pen/Strep (1 mL), and glutamine (1 mL).

### Procedure:

Embryonic mouse hippocampal neurons were cultured according to Kaech, S. and Banker, G. (2006) Nat Protoc 1:2406-2415 and dissected at PO from CD1 mice. Once all the hippocampi were removed, they were placed in a 15 mL conical Falcon tube on ice and brought to a final volume of 4.5 mL with CMF dissection buffer. 0.5 mL of a 2.5% trypsin-EDTA solution was then added, and the mixture was incubated at 37° C. for 15 min. The trypsin solution was gently removed, leaving the tissue at the bottom of the Falcon tube. 5 mL CMF dissection buffer was then added, and after gentle mixing, the tissue was allowed to sediment. This procedure was repeated three times. The hippocampi were then dissociated by adding 1.8 mL platting media and repeatedly pipetting in a glass Pasteur pipette; the dissociation process was repeated 5-10 times. The cells were then passed through a 70 um cell strainer into a 50 mL conical tube to remove clumps and debris, and the neurons were plated on glass coverslips coated with poly-D-lysine/laminin. On DIV 1 neurons were transduced with AAV1 EGFP, WT TDP-43 EGFP, A315T TDP-43 EGFP, or Q331K TDP-43 EGFP. Starting at DIV7 neurons were treated every 48 h (DIV7, 9, 11) with an exemplary compound of the invention at a concentration of 10 times the IC₅₀ value. On DIV12, neurons were fixed in 4% PFA and stained for MAP2 or β-3-tubulin (0.1% Triton-X100 antigen retrieval, block in 10% Donkey Serum, primary overnight 1:1000 (Aves) or 1:500 (Millipore) at 4° C. in 5% Donkey Serum). Imaging was done on the Zeiss microscope at 20× with 6×6 tiling. Neurons were traced and analyzed using NeuronJ.

Results of the neuroprotection assay for exemplary compounds of the invention are summarized in Table 2, wherein A represents an average rescue total dendrite length of >150%; B represents an average rescue total dendrite length of 100-149%; C represents an average rescue total dendrite length of 50-99%; D represents an average rescue total dendrite length of 0-49%; E represents an average rescue total dendrite length of <0%; and ND signifies that the average rescue total dendrite length was not determined.

TABLE 2
Efficacy of Exemplary Compounds of the Invention

| | | Average Additive |
|---|---|---|
| Compound No. | IC₅₀ (nM) | Dendrite Length (%) |
| 100 | B | E |
| 101 | B | C |
| 102 | C | B |
| 103 | D | ND |
| 104 | B | ND |
| 105 | A | ND |
| 106 | B | ND |
| 107 | D | ND |
| 108 | B | ND |
| 109 | D | ND |
| 110 | A | ND |
| 111 | D | ND |
| 112 | D | ND |
| 113 | D | ND |

### Example 29: In Vitro Efficacy Assay of Exemplary Compounds

Exemplary compounds of the invention were evaluated for efficacy in inhibiting TDP-43 inclusions using a concentration-response assay. Briefly, PC12 cells stably expressing a GFP-tagged mutant form of TDP-43 (TDP-43^{Q331K}::eGFP) were pre-treated for 1 hour with exemplary compounds and stressed with 15 μM sodium arsenite for 23 hours to induce TDP-43 aggregation. The inhibitory effect on TDP-43 aggregation was measured using fluorescence microscopy. The ratio of cells with TDP-43 aggregates was calculated based on the total number of cells with detectable GFP expression. A 10-point dose response curve was generated, and the IC₅₀ for each compound tested was determined and is summarized in Table 3 below. In the table, “A” indicates an IC₅₀ of less than or equal to 1.5 μM, “B” indicates an IC₅₀ range from 1.5 μM to 4 μM; “C” indicates an IC₅₀ range from 4 μM to 7 μM; “D” indicates an IC₅₀ range from 7 μM to 9.9 μM; “E” indicates an IC₅₀ greater than or equal to 10 μM; and “F” indicates that the IC₅₀ was not determined.

TABLE 3
Efficacy of Exemplary Compounds of the Invention

| Compound No. | Average IC₅₀ (nM) |
|---|---|
| 101 | A |
| 120 | F |
| 122 | A |
| 123 | B |
| 124 | C |
| 125 | A |
| 126 | E |
| 127 | A |
| 128 | A |
| 129 | E |
| 130 | B |
| 131 | E |
| 132 | B |
| 133 | B |
| 134 | B |
| 135 | A |
| 137 | B |
| 138 | A |
| 139 | B |
| 140 | A |
| 141 | A |
| 142 | E |
| 143 | A |
| 144 | A |
| 145 | B |
| 146 | B |
| 147 | E |
| 148 | A |
| 149 | A |
| 150 | A |
| 151 | A |
| 152 | E |
| 153 | B |
| 154 | B |
| 155 | B |
| 156 | B |
| 157 | E |
| 158 | C |
| 159 | B |
| 160 | A |
| 161 | A |
| 162 | B |
| 163 | A |
| 164 | B |
| 165 | B |
| 166 | E |
| 167 | A |
| 168 | A |
| 169 | A |
| 170 | A |
| 171 | A |
| 172 | B |
| 173 | B |
| 174 | B |
| 175 | E |
| 176 | E |
| 177 | A |
| 178 | A |
| 179 | C |
| 180 | E |
| 181 | D |
| 200 | B |
| 201 | A |
| 202 | A |
| 203 | B |
| 204 | A |
| 205 | B |
| 206 | A |
| 207 | A |
| 208 | C |
| 209 | C |
| 210 | E |
| 211 | B |
| 212 | E |
| 213 | E |
| 214 | E |
| 215 | E |
| 216 | B |
| 217 | A |
| 218 | E |
| 219 | A |
| 220 | E |
| 221 | B |
| 222 | E |
| 223 | B |
| 224 | B |
| 225 | E |
| 226 | F |
| 227 | B |
| 228 | E |
| 229 | B |
| 230 | A |
| 231 | B |
| 232 | A |
| 233 | A |
| 234 | E |
| 235 | B |
| 236 | E |
| 237 | B |
| 238 | B |
| 239 | E |
| 240 | E |
| 241 | E |
| 242 | B |
| 243 | A |
| 244 | E |
| 245 | F |
| 246 | B |
| 247 | C |
| 248 | A |
| 249 | A |
| 250 | A |
| 251 | E |
| 252 | E |
| 253 | B |
| 254 | E |
| 255 | E |
| 256 | E |
| 257 | C |
| 258 | E |
| 259 | E |
| 260 | C |
| 261 | B |
| 262 | E |
| 263 | E |
| 264 | B |
| 265 | A |
| 266 | C |
| 267 | B |
| 268 | B |
| 269 | E |
| 270 | E |
| 271 | A |
| 272 | E |
| 273 | B |
| 274 | E |
| 275 | E |
| 276 | E |
| 277 | E |
| 278 | C |
| 279 | E |
| 280 | E |
| 281 | E |
| 282 | E |
| 283 | E |
| 284 | E |
| 285 | B |
| 286 | B |
| 287 | C |
| 288 | A |
| 289 | E |
| 290 | E |
| 291 | B |
| 292 | E |
| 293 | E |
| 294 | E |
| 295 | C |
| 296 | B |
| 297 | B |
| 298 | B |
| 299 | E |
| 300 | B |
| 301 | E |
| 302 | E |
| 303 | E |
| 304 | E |
| 305 | A |
| 306 | A |
| 307 | A |
| 308 | E |
| 309 | E |
| 310 | C |
| 311 | D |
| 312 | B |
| 313 | E |
| 314 | A |
| 315 | A |
| 316 | C |
| 317 | B |
| 318 | A |
| 320 | E |
| 321 | A |
| 322 | B |
| 323 | A |
| 324 | B |
| 325 | E |
| 326 | B |
| 327 | D |
| 328 | C |
| 329 | A |
| 330 | A |
| 331 | B |
| 332 | A |
| 333 | E |
| 334 | A |
| 335 | A |
| 336 | A |
| 337 | A |
| 338 | A |
| 339 | A |
| 340 | A |
| 341 | A |
| 342 | E |
| 343 | B |
| 344 | E |
| 345 | B |
| 350 | A |
| 351 | A |
| 352 | A |
| 353 | A |
| 354 | A |
| 355 | A |
| 356 | A |
| 357 | A |
| 358 | A |
| 359 | A |
| 360 | A |
| 361 | F |
| 362 | F |
| 363 | A |
| 364 | A |
| 365 | B |
| 400 | E |
| 401 | C |
| 402 | E |
| 403 | B |
| 404 | A |
| 405 | C |
| 406 | A |
| 407 | E |
| 408 | A |
| 409 | B |
| 410 | B |
| 411 | A |
| 412 | B |
| 413 | E |
| 414 | E |
| 415 | E |
| 416 | E |
| 417 | E |
| 418 | E |
| 419 | E |
| 420 | E |
| 421 | C |
| 422 | E |
| 423 | A |
| 424 | C |
| 425 | E |
| 426 | E |

### EQUIVALENTS

It will be recognized that one or more features of any embodiments disclosed herein may be combined and/or rearranged within the scope of the invention to produce further embodiments that are also within the scope of the invention.

Those skilled in the art will recognize, or be able to ascertain using no more than routine experimentation, many equivalents to the specific embodiments of the invention described herein. Such equivalents are intended to be within the scope of the present invention.

Although the invention has been described and illustrated in the foregoing illustrative embodiments, it is understood that the present disclosure has been made only by way of example, and that numerous changes in the details of implementation of the invention can be made without departing from the spirit and scope of the invention, which is limited only by the claims that follow. Features of the disclosed embodiments can be combined and/or rearranged in various ways within the scope and spirit of the invention to produce further embodiments that are also within the scope of the invention. Those skilled in the art will recognize, or be able to ascertain, using no more than routine experimentation, numerous equivalents to the specific embodiments described specifically in this disclosure. Such equivalents are intended to be encompassed in the scope of the following claims.

All patents, patent applications and publications cited herein are hereby incorporated by reference in their entirety. The disclosures of these publications in their entireties are hereby incorporated by reference into this application in order to more fully describe the state of the art as known to those skilled therein as of the date of the invention described and claimed herein.

1. A compound of Formula (I): 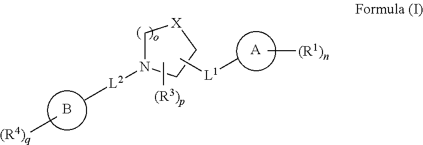
each of Ring A and Ring B is independently cycloalkyl, heterocyclyl, aryl, or heteroaryl;
X is C(R′), C(R′)(R″), N, or NR^{A};
each of L¹ and L² is independently a bond, —C₁-C₆ alkyl-, —C₂-C₆ alkenyl-, —C₂-C₆ alkynyl-, —C₁-C₆ heteroalkyl-, —C(O)—, —OC(O)—, —C(O)O—, —OC(O)O—, —C(O)NR^{A}—, —NR^{A}C(O)—, —C(O)NR^{A}—C₁-C₆ alkyl, —C₁-C₆ alkyl-C(O)NR^{A}—, —NR^{A}C(O)—C₁-C₆ alkyl-, —C₁-C₆ alkyl-NR^{A}C(O)—, —C(O)NR^{A}—C₁-C₆ heteroalkyl-, —C₁-C₆ heteroalkyl-C(O)NR^{A}—, —NR^{A}C(O)—C₁-C₆ heteroalkyl-, —C₁-C₆ heteroalkyl-NR^{A}C(O)—, —C₁-C₆ alkyl-C(O)—, —C(O)—C₁-C₆ alkyl, —C₁-C₆ heteroalkyl-C(O)—, —C(O)—C₁-C₆ heteroalkyl, —C(O)—C₁-C₆ alkyl-C(O)NR^{A}—, —S(O)ₓ—, —OS(O)ₓ, —C(O)NR^{A}S(O)ₓ—, —NR^{A}S(O)ₓ—, or —S(O)ₓNR^{A}—, each of which is optionally substituted with 1-5 R⁵;
each of R¹ and R⁴ is independently C₁-C₆ alkyl, C₂-C₆ alkenyl, C₂-C₆ alkynyl, C₁-C₆ heteroalkyl, C₁-C₆ haloalkyl, halo, cyano, cycloalkyl, heterocyclyl, aryl, heteroaryl, —OR^{B}, —NR^{A}R^{C}, —NR^{A}C(O)R^{D}, —S(O)ₓR^{E}, —OS(O)ₓR^{E}, —C(O)NR^{A}S(O)ₓR^{E}, —NR^{A}S(O)ₓR^{E}, or —S(O)ₓNR^{A}, each of which is optionally substituted with 1-5 R⁶;
R³ is H, C₁-C₆ alkyl, C₂-C₆ alkenyl, C₂-C₆ alkynyl, C₁-C₆ heteroalkyl, C₁-C₆ haloalkyl, halo, cyano, nitro, cycloalkyl, heterocyclyl, aryl, heteroaryl, —OR^{B}, —NR^{A}R^{C}, —C(O)R^{D}, —C(O)OR^{B}, —C(O)NR^{A}R^{C}, —NR^{A}C(O)R^{D}, —NR^{A}C(O)NR^{B}R^{C}, —SR^{E}, —S(O)ₓR^{E}, —NR^{A}S(O)ₓR^{E}, or —S(O)ₓNR^{A}R^{C}, each of which is optionally substituted with 1-5 R⁷; or
or two R³, taken together with the atoms to which they are attached, form a ring (e.g., a 5-7 membered ring), optionally substituted with 1-5 R⁷;
each of R′ and R″ is independently H, C₁-C₆ alkyl, C₁-C₆ heteroalkyl, C₁-C₆ haloalkyl, halo, cyano, cycloalkyl, or heterocyclyl, each of which is optionally substituted with 1-5 R⁷;
each of R⁵, R⁶, and R⁷ is independently C₁-C₆ alkyl, C₂-C₆ alkenyl, C₂-C₆ alkynyl, C₁-C₆ heteroalkyl, C₁-C₆ haloalkyl, halo, cyano, cycloalkyl, heterocyclyl, aryl, heteroaryl, —OR^{B}, —C(O)R^{D}, —C(O)OR^{B}, —C(O)NR^{A}R^{C}, or —SR^{E}, each of which is optionally substituted with 1-5 R⁸;
each R^{A}, R^{B}, R^{C}, R^{D}, or R^{E} is independently H, C₁-C₆ alkyl, C₁-C₆ heteroalkyl, C₁-C₆ haloalkyl, cycloalkyl, heterocyclyl, aryl, heteroaryl, arylalkyl, heteroarylalkyl, cycloalkylalkyl, or heterocycloalkyl, each of which is optionally substituted with 1-4 R⁸;
or R^{A} and R^{C}, together with the atoms to which each is attached, form a heterocyclyl ring optionally substituted with 1-4 R⁸;
each R⁸ is independently C₁-C₆ alkyl, C₂-C₆ alkenyl, C₂-C₆ alkynyl, C₁-C₆ heteroalkyl, C₁-C₆ haloalkyl, cycloalkyl, heterocyclyl, aryl, heteroaryl, halo, cyano, or nitro, each of which is optionally substituted with 1-5 R⁹;
each R⁹ is C₁-C₆ alkyl, halo, hydroxy, cycloalkyl, alkoxy, keto, cyano, or nitro;
each of n and q is independently 0, 1, 2, 3, 4, 5, or 6;
o is 1, 2, or 3;
p is 0, 1, 2, 3 or 4; and
x is 0, 1, or 2;
wherein when L¹ is connected to X, X is C(R′) or N,
provided the compound is not

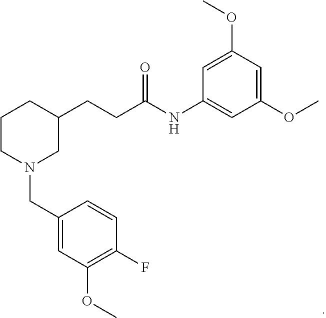 [SMILES: COc1cc(NC(=O)CCC2CCCN(Cc3ccc(F)c(OC)c3)C2)cc(OC)c1]

2. The compound of claim 1, wherein Ring A is aryl.

3. The compound of claim 1, wherein Ring A is phenyl.

4. The compound of claim 1, wherein Ring A is heteroaryl.

5. The compound of claim 4, wherein Ring A is a monocyclic heteroaryl or bicyclic heteroaryl.

6. The compound of claim 4, wherein Ring A is indolyl, indolinyl, indazolyl, benzofuranyl, benzoimidazolyl, benzooxazolyl, or benzothiazolyl.

7. The compound of claim 4, wherein Ring A is indolyl.

8. The compound of claim 4, wherein Ring A is pyrrolyl, furanyl, or pyridyl.

9. The compound of claim 1, wherein n is 0.

10. The compound of claim 1, wherein n is 1, 2, or 3, and R¹ is C₁-C₆ alkyl (e.g., methyl or ethyl), halo, cyano, or —OR^{B}.

11. The compound of claim 1, wherein Ring B is aryl.

12. The compound of claim 1, wherein Ring B is phenyl.

13. The compound of claim 1, wherein Ring B is heteroaryl.

14. The compound of claim 13, wherein Ring B is a bicyclic heteroaryl.

15. The compound of claim 14, wherein Ring B is indolyl, benzofuranyl, benzoimidazolyl, or benzothiazolyl.

16. The compound of claim 1, wherein q is 0.

17. The compound of claim 1, wherein q is 1, 2, or 3, and R⁴ is C₁-C₆ alkyl, halo, cyano, —C(O)OR^{B}.

18. The compound of claim 1, wherein X is C(R′)(R″).

19. The compound of claim 1, wherein each of R′ and R″ is independently H.

20. The compound of claim 1, wherein X is NR^{A}, and R^{A} is H.

21. The compound of claim 1, wherein each of L¹ and L² is independently a bond, C₁-C₆ alkyl, C₁-C₆ heteroalkyl, —C(O)—, —C(O)NR^{A}—, —NR^{A}C(O)—, —C(O)NR^{A}—C₁-C₆ alkyl, —NR^{A}C(O)—C₁-C₆ alkyl, —NR^{A}C(O)—C₁-C₆ heteroalkyl, —C(O)—C₁-C₆ alkyl, C₁-C₆ alkyl-C(O)—, C₁-C₆ alkyl-NR^{A}C(O)—, —S(O)ₓ—, —OS(O)ₓ, —C(O)NR^{A}S(O)ₓ—, —NR^{A}S(O)ₓ—, or —S(O)ₓNR^{A}—, each of which is optionally substituted with 1-5 R⁵.

22. The compound of claim 1, wherein L¹ is C₁-C₆ alkyl or C₁-C₆ alkyl-NR^{A}C(O)—.

23. The compound of claim 1, wherein L¹ is C₁-C₆ alkyl-NR^{A}C(O)— and R^{A} is H, C₁-C₆ alkyl, C₁-C₆ heteroalkyl, C₁-C₆ haloalkyl, cycloalkyl, aryl, cycloalkylalkyl, or arylalkyl.

24. (canceled)

25. The compound of claim 1, wherein L² is a bond, C₁-C₆ alkyl, —S(O)ₓ— (e.g., S(O)₂), or —C(O)—C₁-C₆ alkyl, each of which is optionally substituted with 1-5 R⁵.

26. The compound of claim 1, wherein L² is C₁-C₆ alkyl.

27. The compound of claim 1, wherein p is 0.

28. The compound of claim 1, wherein p is 2 and each R³ is independently C₁-C₆ alkyl, wherein both R³ are joined together to form a 6- or 7-membered ring.

29. The compound of claim 1, wherein o is 2.

30. The compound of claim 1, wherein the compound of Formula (I) is a compound of Formula (I-d), Formula (I-e), or Formula (I-f): 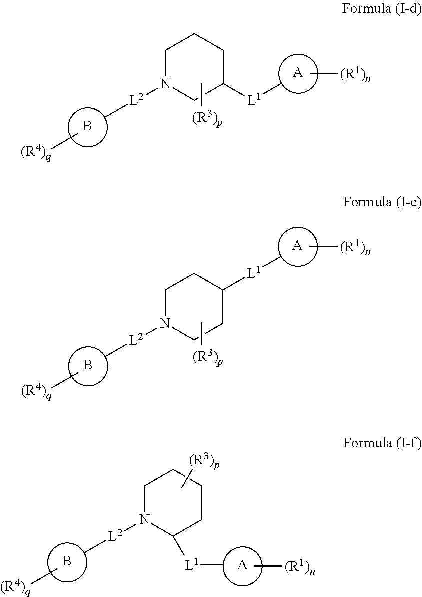
or a pharmaceutically acceptable salt thereof.

31. The compound of claim 1, wherein the compound of Formula (I) is a compound of Formula (I-g), Formula (I-h), or Formula (I-i): 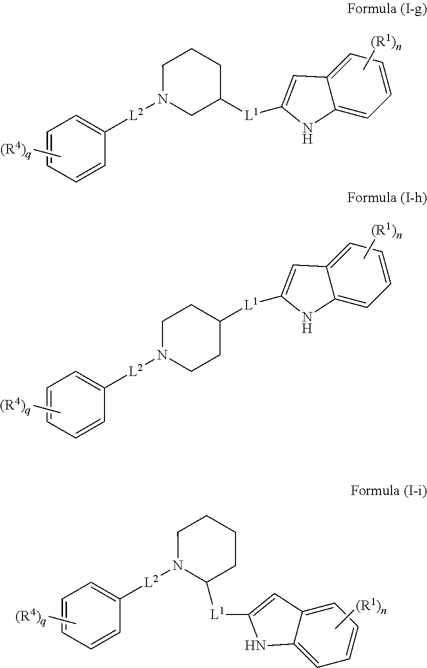
or a pharmaceutically acceptable salt thereof.

32. The compound of claim 1, wherein the compound of Formula (I) is selected from

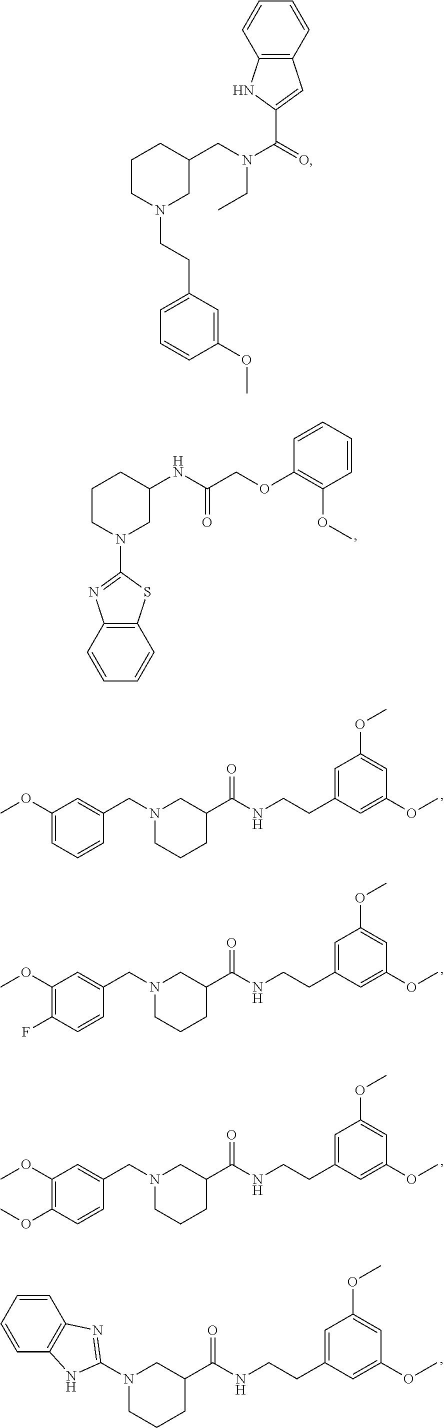

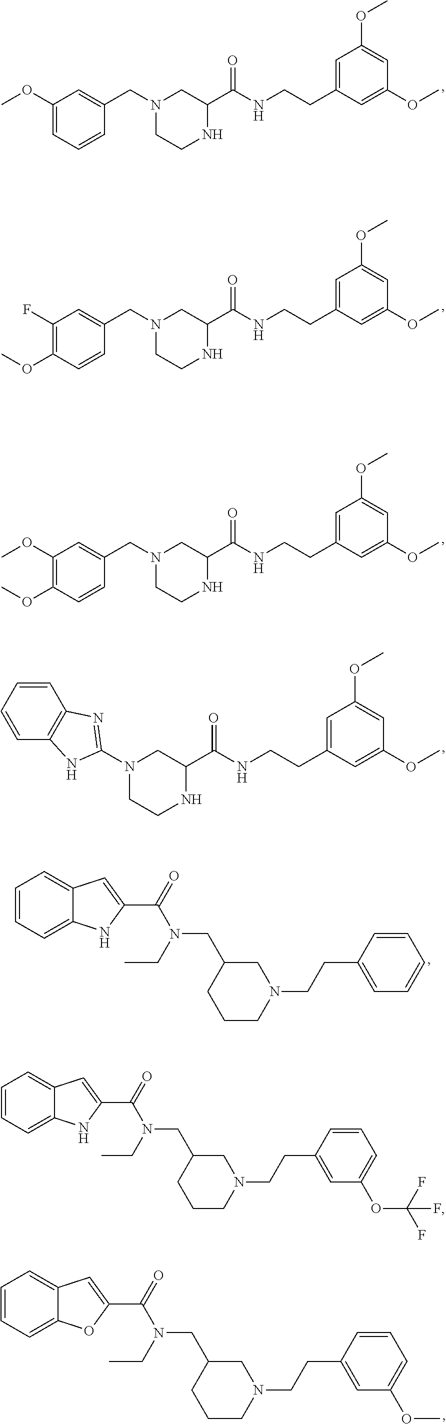

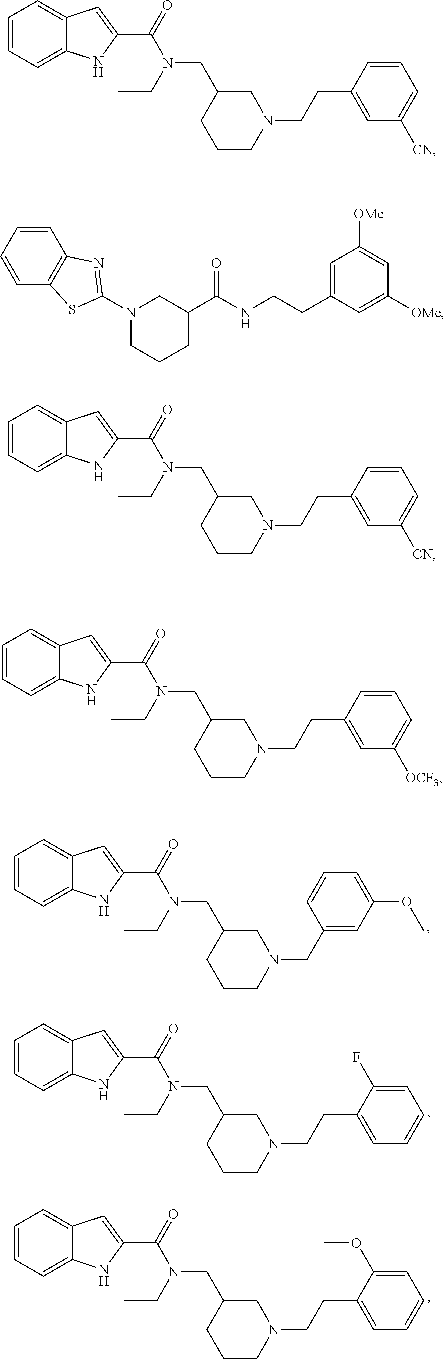

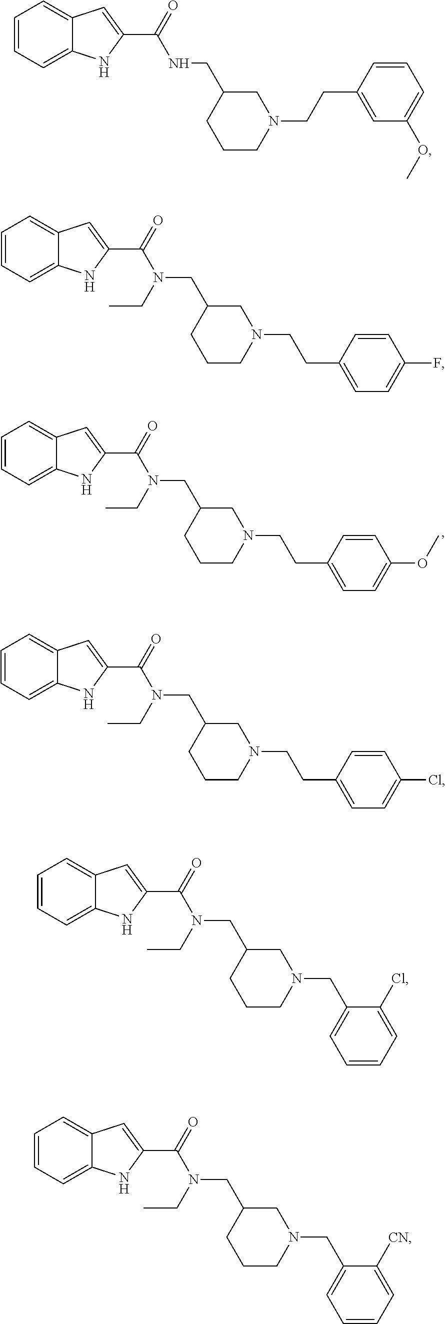 [SMILES: COc1cccc(CCN2CCCC(CNC(=O)c3cc4ccccc4[nH]3)C2)c1 ; CCN(CC1CCCN(CCc2ccc(F)cc2)C1)C(=O)c1cc2ccccc2[nH]1 ; CCN(CC1CCCN(CCc2ccc(OC)cc2)C1)C(=O)c1cc2ccccc2[nH]1 ; CCN(CC1CCCN(CCc2ccc(Cl)cc2)C1)C(=O)c1cc2ccccc2[nH]1 ; CCN(CC1CCCN(Cc2ccccc2Cl)C1)C(=O)c1cc2ccccc2[nH]1 ; CCN(CC1CCCN(Cc2ccccc2C#N)C1)C(=O)c1cc2ccccc2[nH]1]

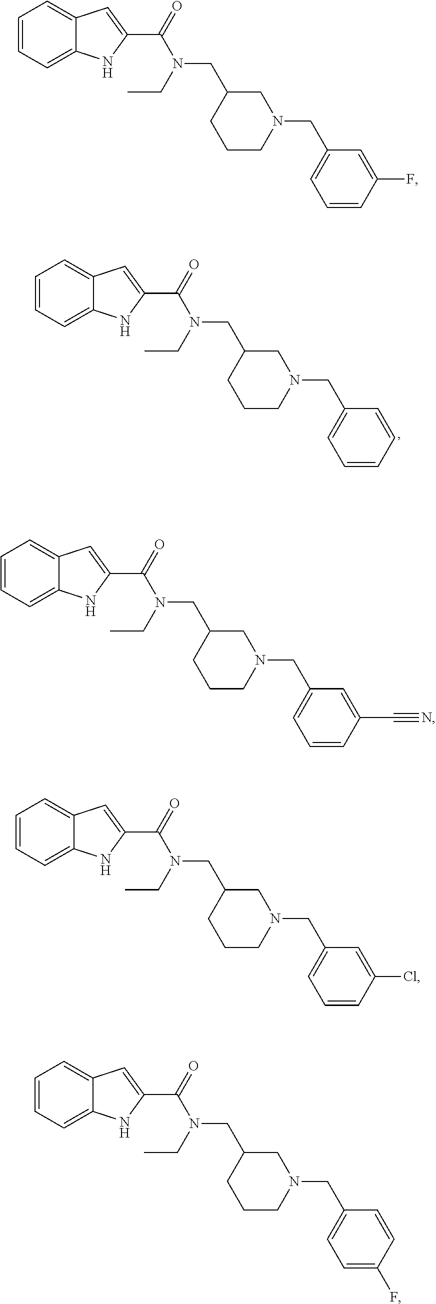 [SMILES: CCN(CC1CCCN(Cc2cccc(F)c2)C1)C(=O)c1cc2ccccc2[nH]1 ; CCN(CC1CCCN(Cc2ccccc2)C1)C(=O)c1cc2ccccc2[nH]1 ; CCN(CC1CCCN(Cc2cccc(C#N)c2)C1)C(=O)c1cc2ccccc2[nH]1 ; CCN(CC1CCCN(Cc2cccc(Cl)c2)C1)C(=O)c1cc2ccccc2[nH]1 ; CCN(CC1CCCN(Cc2ccc(F)cc2)C1)C(=O)c1cc2ccccc2[nH]1]

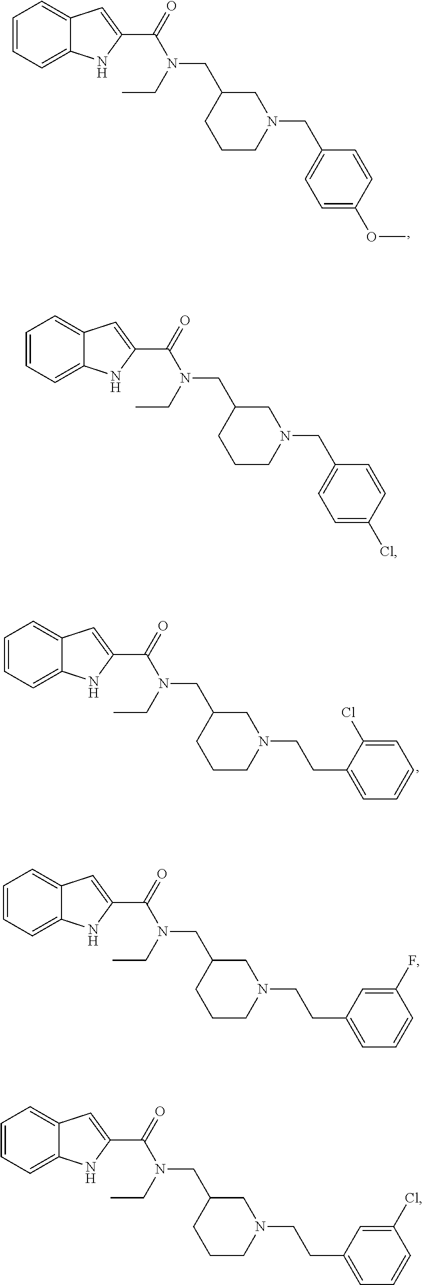 [SMILES: CCN(CC1CCCN(Cc2ccc(OC)cc2)C1)C(=O)c1cc2ccccc2[nH]1 ; CCN(CC1CCCN(Cc2ccc(Cl)cc2)C1)C(=O)c1cc2ccccc2[nH]1 ; CCN(CC1CCCN(CCc2ccccc2Cl)C1)C(=O)c1cc2ccccc2[nH]1 ; CCN(CC1CCCN(CCc2cccc(F)c2)C1)C(=O)c1cc2ccccc2[nH]1 ; CCN(CC1CCCN(CCc2cccc(Cl)c2)C1)C(=O)c1cc2ccccc2[nH]1]

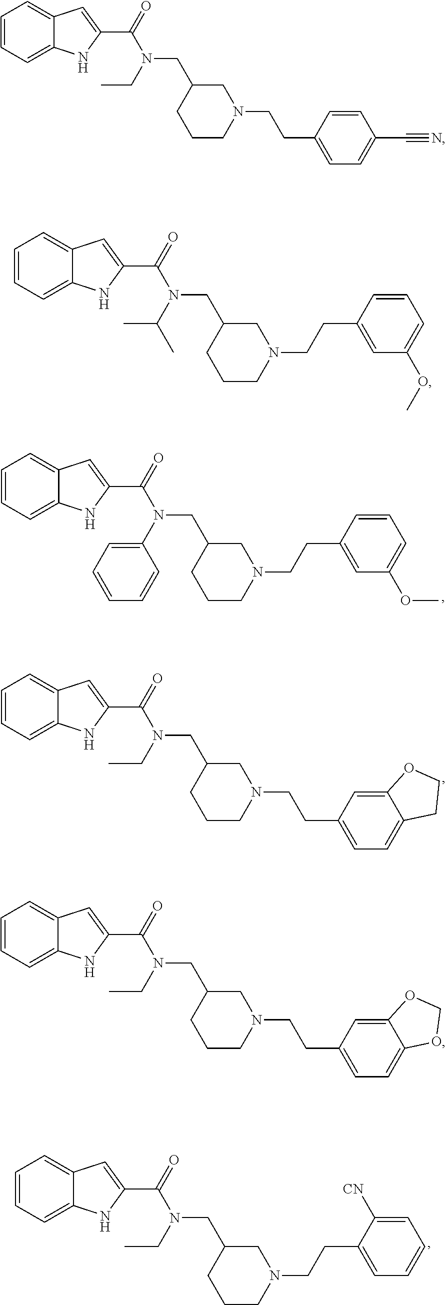

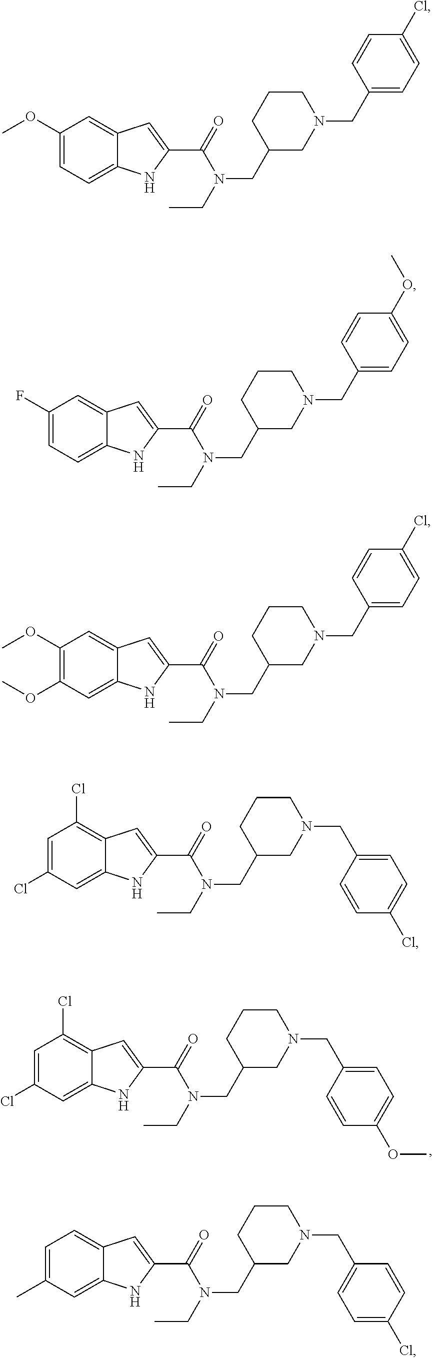

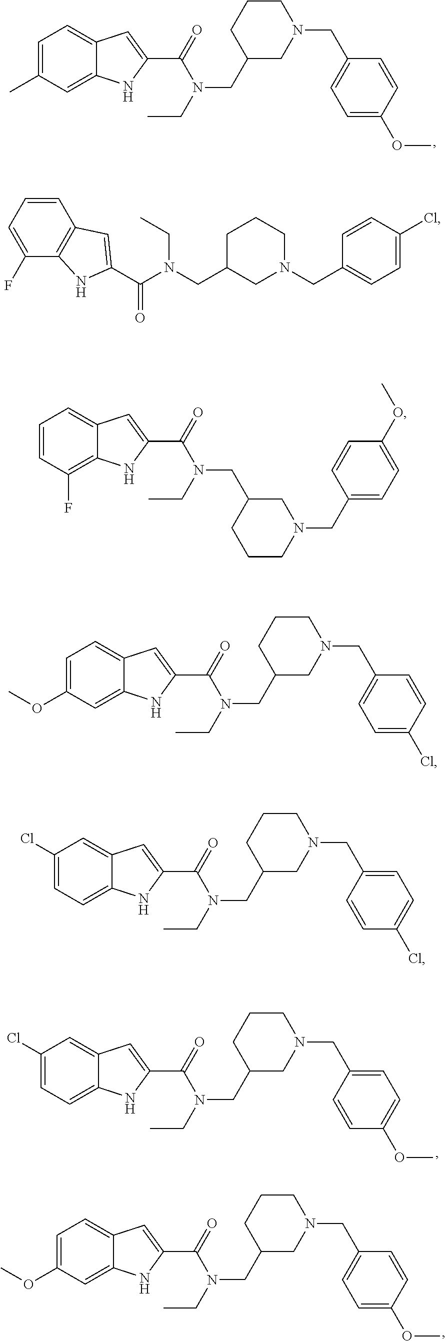

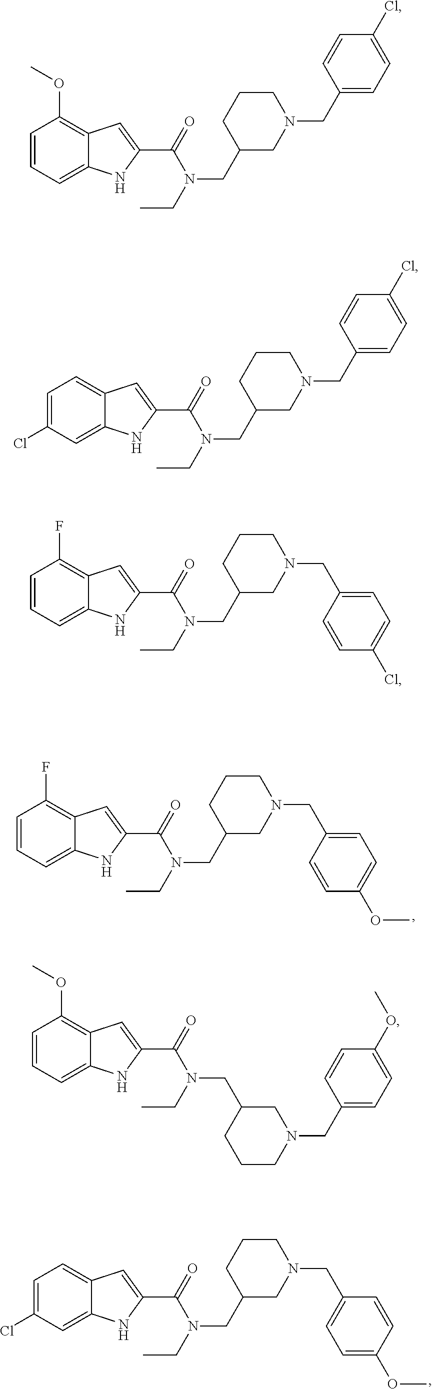 [SMILES: CCN(CC1CCCN(Cc2ccc(Cl)cc2)C1)C(=O)c1cc2c(OC)cccc2[nH]1 ; CCN(CC1CCCN(Cc2ccc(Cl)cc2)C1)C(=O)c1cc2ccc(Cl)cc2[nH]1 ; CCN(CC1CCCN(Cc2ccc(Cl)cc2)C1)C(=O)c1cc2c(F)cccc2[nH]1 ; CCN(CC1CCCN(Cc2ccc(OC)cc2)C1)C(=O)c1cc2c(F)cccc2[nH]1 ; CCN(CC1CCCN(Cc2ccc(OC)cc2)C1)C(=O)c1cc2c(OC)cccc2[nH]1 ; CCN(CC1CCCN(Cc2ccc(OC)cc2)C1)C(=O)c1cc2ccc(Cl)cc2[nH]1]

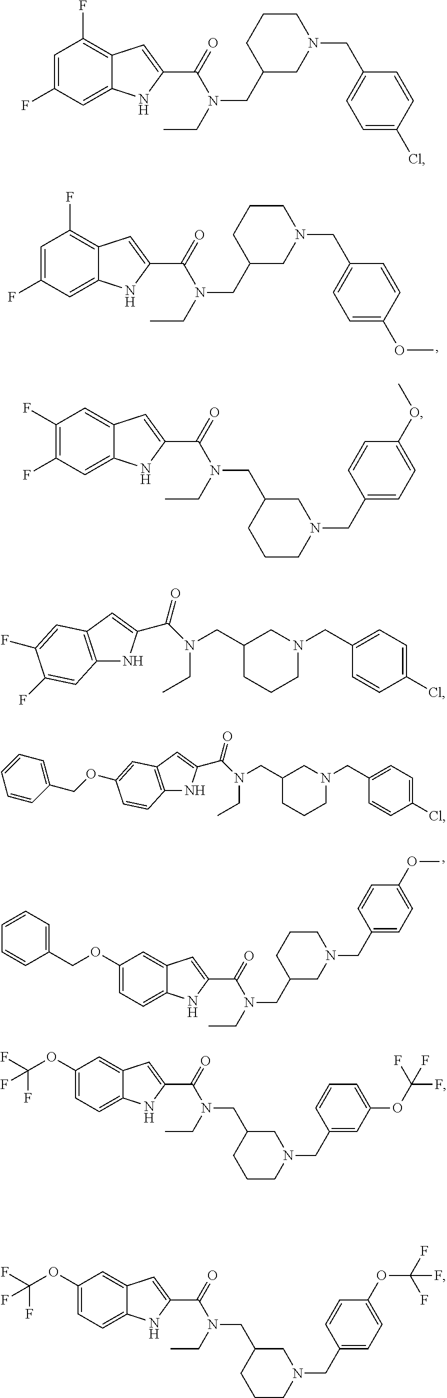

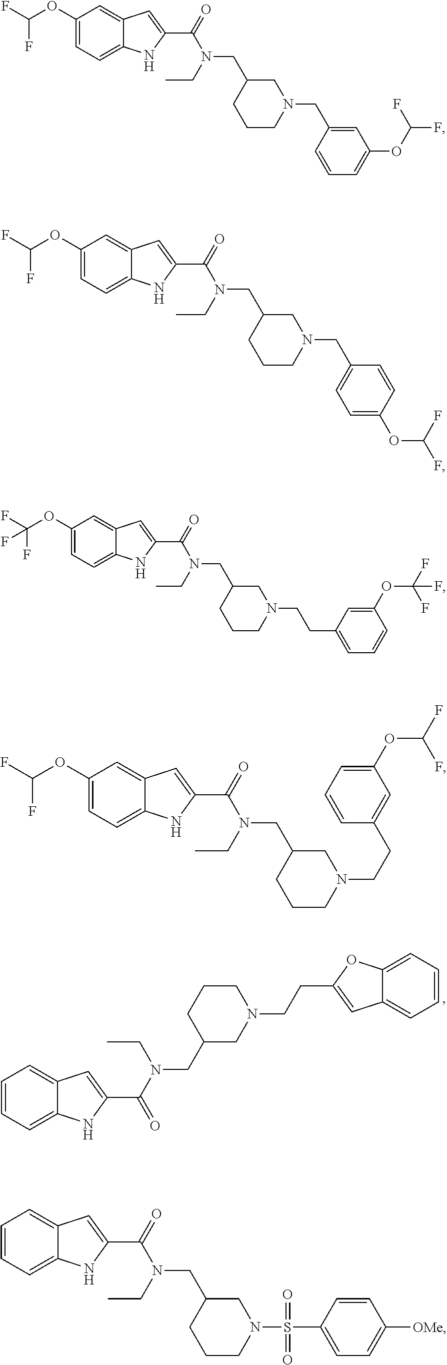 [SMILES: CCN(CC1CCCN(Cc2cccc(OC(F)F)c2)C1)C(=O)c1cc2cc(OC(F)F)ccc2[nH]1 ; CCN(CC1CCCN(Cc2ccc(OC(F)F)cc2)C1)C(=O)c1cc2cc(OC(F)F)ccc2[nH]1 ; CCN(CC1CCCN(CCc2cccc(OC(F)(F)F)c2)C1)C(=O)c1cc2cc(OC(F)(F)F)ccc2[nH]1 ; CCN(CC1CCCN(CCc2cccc(OC(F)F)c2)C1)C(=O)c1cc2cc(OC(F)F)ccc2[nH]1 ; CCN(CC1CCCN(CCc2cc3ccccc3o2)C1)C(=O)c1cc2ccccc2[nH]1 ; CCN(CC1CCCN(S(=O)(=O)c2ccc(OC)cc2)C1)C(=O)c1cc2ccccc2[nH]1]

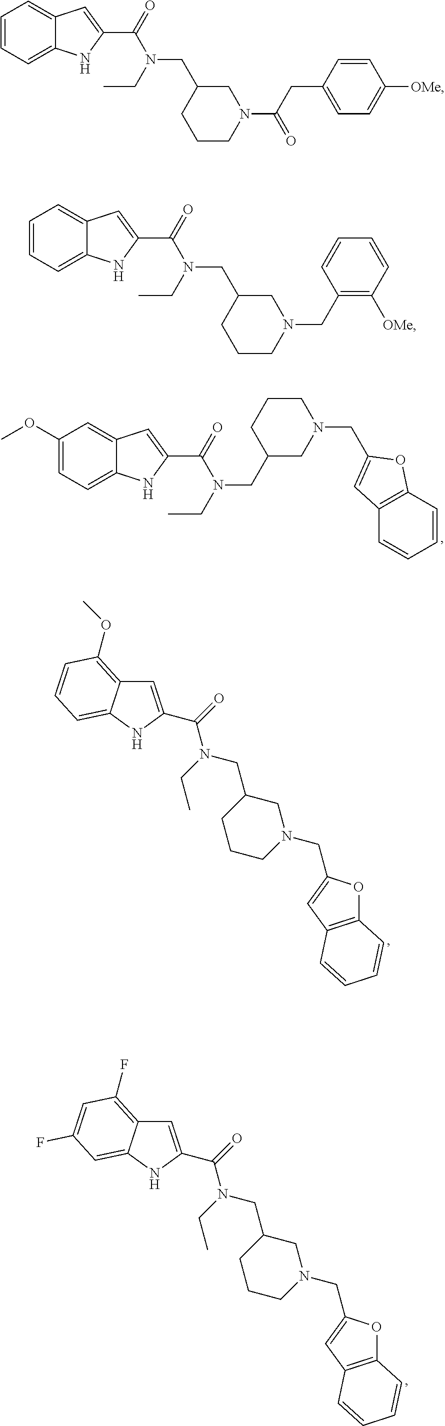

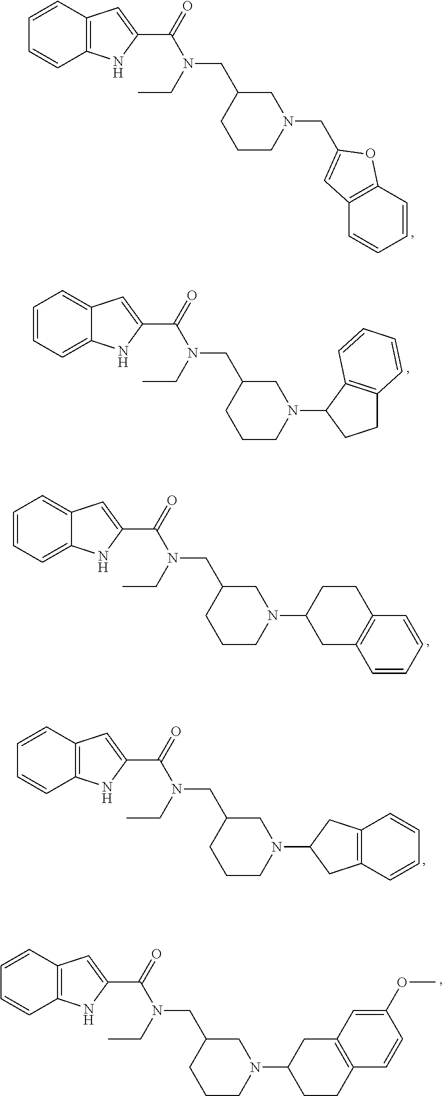 [SMILES: CCN(CC1CCCN(Cc2cc3ccccc3o2)C1)C(=O)c1cc2ccccc2[nH]1 ; CCN(CC1CCCN(C2CCc3ccccc32)C1)C(=O)c1cc2ccccc2[nH]1 ; CCN(CC1CCCN(C2CCc3ccccc3C2)C1)C(=O)c1cc2ccccc2[nH]1 ; CCN(CC1CCCN(C2Cc3ccccc3C2)C1)C(=O)c1cc2ccccc2[nH]1 ; CCN(CC1CCCN(C2CCc3ccc(OC)cc3C2)C1)C(=O)c1cc2ccccc2[nH]1]

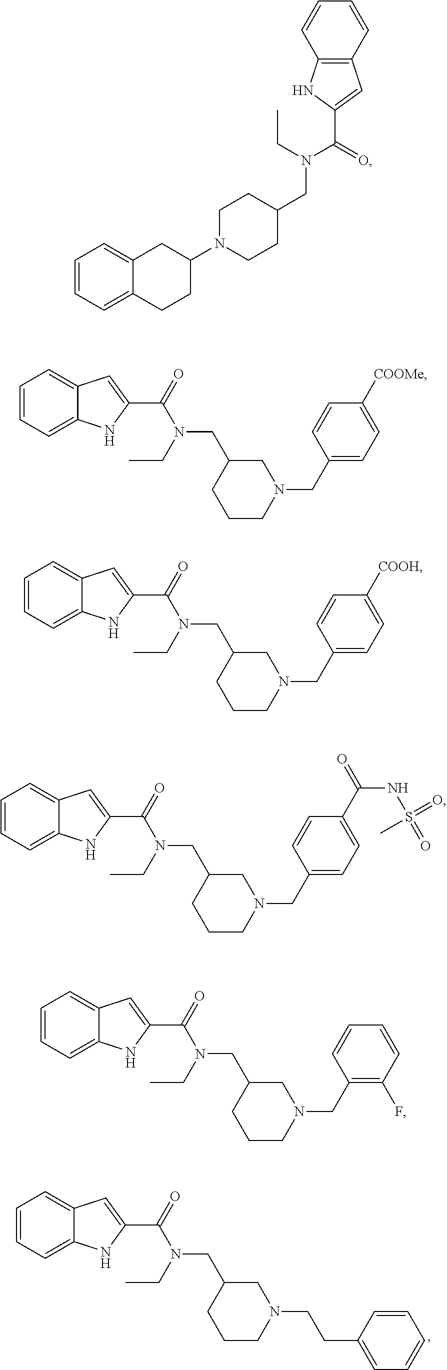 [SMILES: CCN(CC1CCN(C2CCc3ccccc3C2)CC1)C(=O)c1cc2ccccc2[nH]1 ; CCN(CC1CCCN(Cc2ccc(C(=O)OC)cc2)C1)C(=O)c1cc2ccccc2[nH]1 ; CCN(CC1CCCN(Cc2ccc(C(=O)O)cc2)C1)C(=O)c1cc2ccccc2[nH]1 ; CCN(CC1CCCN(Cc2ccc(C(=O)NS(C)(=O)=O)cc2)C1)C(=O)c1cc2ccccc2[nH]1 ; CCN(CC1CCCN(Cc2ccccc2F)C1)C(=O)c1cc2ccccc2[nH]1 ; CCN(CC1CCCN(CCc2ccccc2)C1)C(=O)c1cc2ccccc2[nH]1]

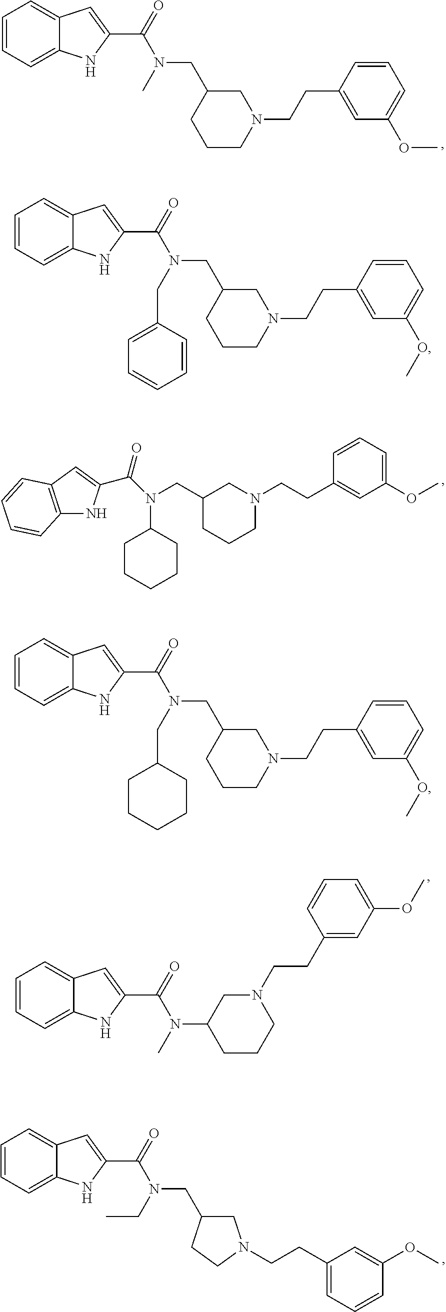 [SMILES: COc1cccc(CCN2CCCC(CN(C)C(=O)c3cc4ccccc4[nH]3)C2)c1 ; COc1cccc(CCN2CCCC(CN(Cc3ccccc3)C(=O)c3cc4ccccc4[nH]3)C2)c1 ; COc1cccc(CCN2CCCC(CN(C(=O)c3cc4ccccc4[nH]3)C3CCCCC3)C2)c1 ; COc1cccc(CCN2CCCC(CN(CC3CCCCC3)C(=O)c3cc4ccccc4[nH]3)C2)c1 ; COc1cccc(CCN2CCCC(N(C)C(=O)c3cc4ccccc4[nH]3)C2)c1 ; CCN(CC1CCN(CCc2cccc(OC)c2)C1)C(=O)c1cc2ccccc2[nH]1]

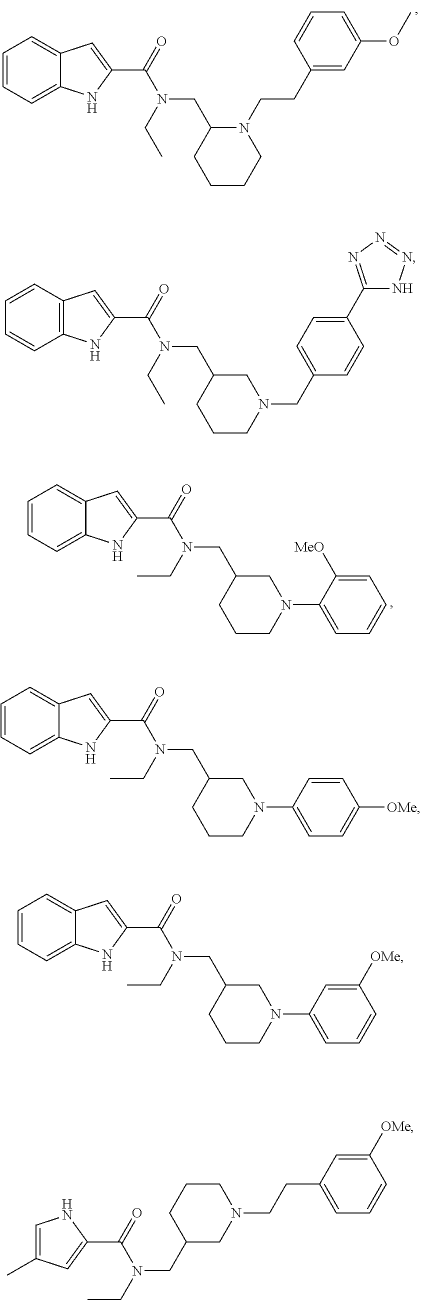

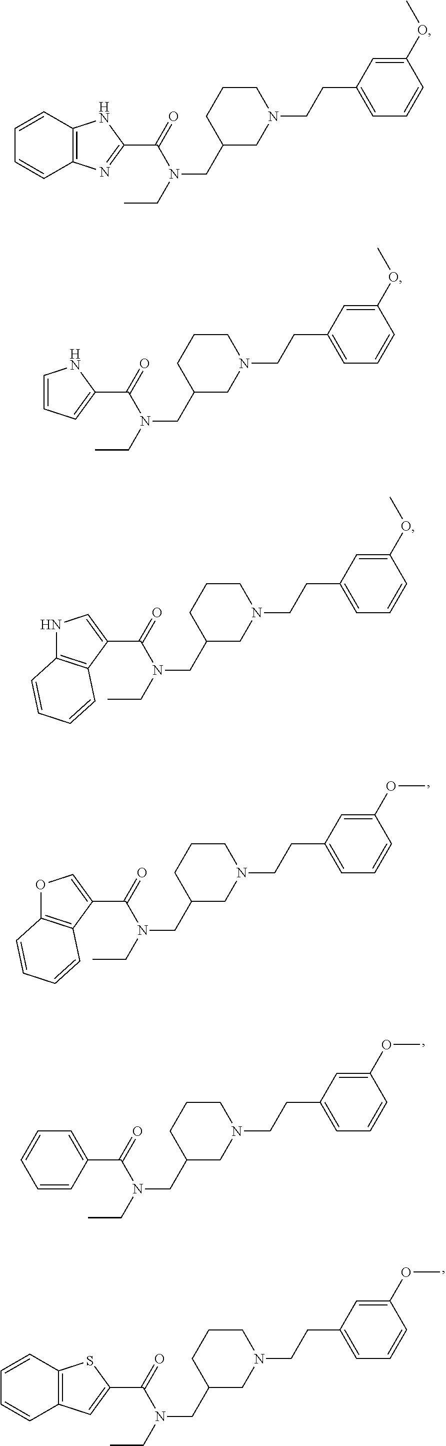 [SMILES: CCN(CC1CCCN(CCc2cccc(OC)c2)C1)C(=O)c1nc2ccccc2[nH]1 ; CCN(CC1CCCN(CCc2cccc(OC)c2)C1)C(=O)c1ccc[nH]1 ; CCN(CC1CCCN(CCc2cccc(OC)c2)C1)C(=O)c1c[nH]c2ccccc12 ; CCN(CC1CCCN(CCc2cccc(OC)c2)C1)C(=O)c1coc2ccccc12 ; CCN(CC1CCCN(CCc2cccc(OC)c2)C1)C(=O)c1ccccc1 ; CCN(CC1CCCN(CCc2cccc(OC)c2)C1)C(=O)c1cc2ccccc2s1]

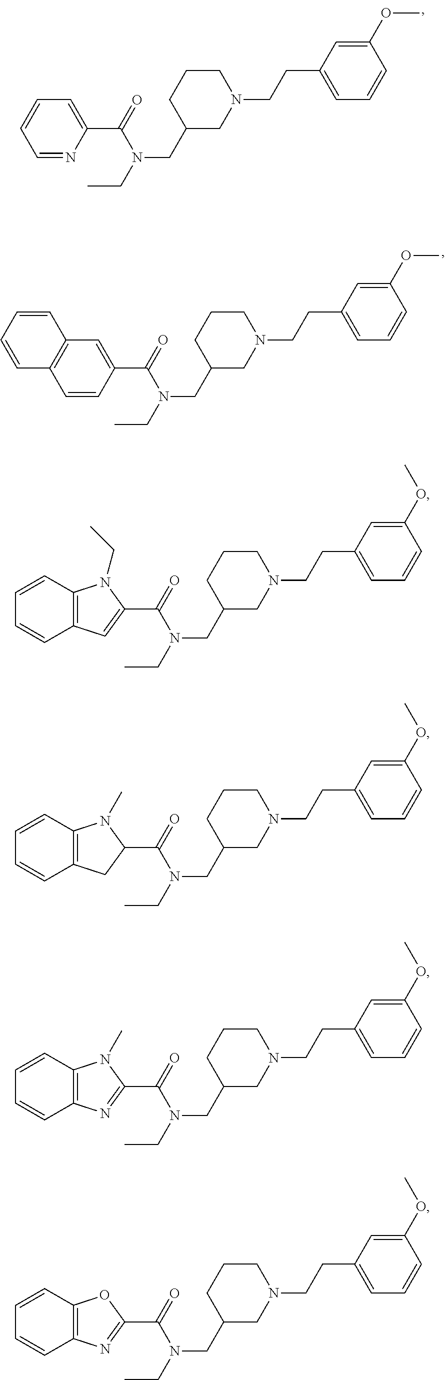 [SMILES: CCN(CC1CCCN(CCc2cccc(OC)c2)C1)C(=O)c1ccccn1 ; CCN(CC1CCCN(CCc2cccc(OC)c2)C1)C(=O)c1ccc2ccccc2c1 ; CCN(CC1CCCN(CCc2cccc(OC)c2)C1)C(=O)c1cc2ccccc2n1CC ; CCN(CC1CCCN(CCc2cccc(OC)c2)C1)C(=O)C1Cc2ccccc2N1C ; CCN(CC1CCCN(CCc2cccc(OC)c2)C1)C(=O)c1nc2ccccc2n1C ; CCN(CC1CCCN(CCc2cccc(OC)c2)C1)C(=O)c1nc2ccccc2o1]

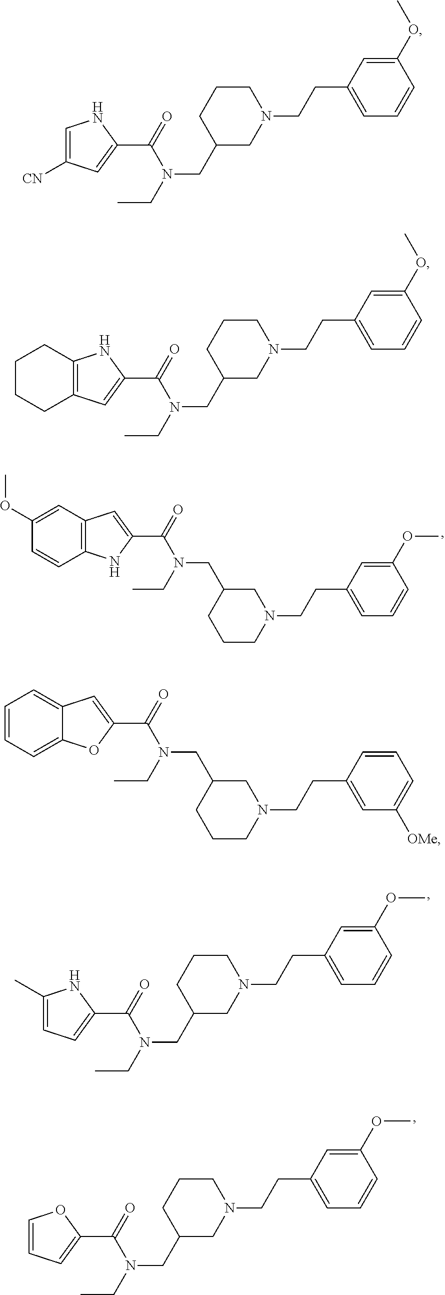

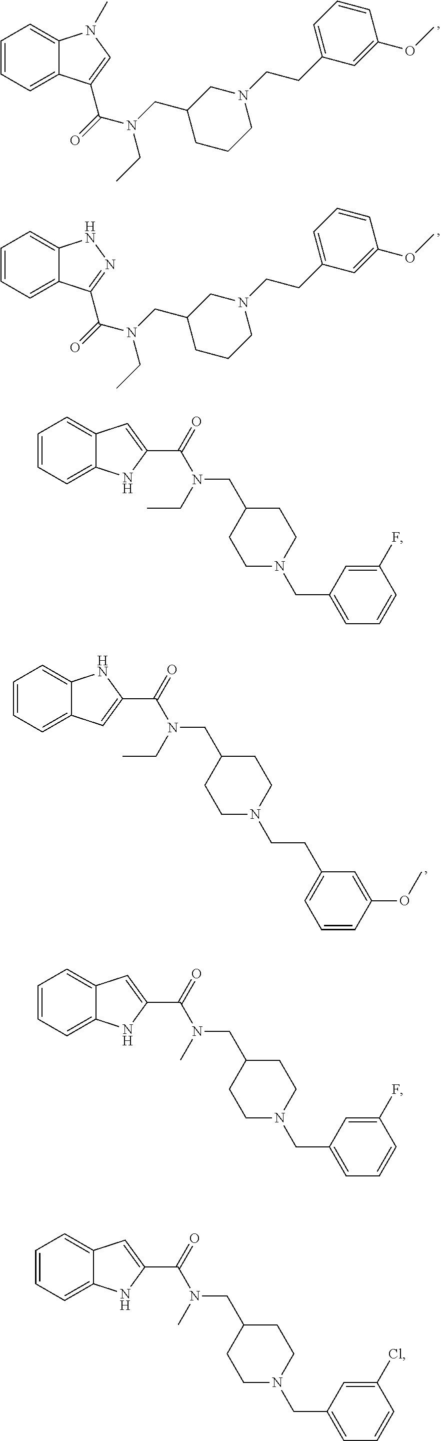

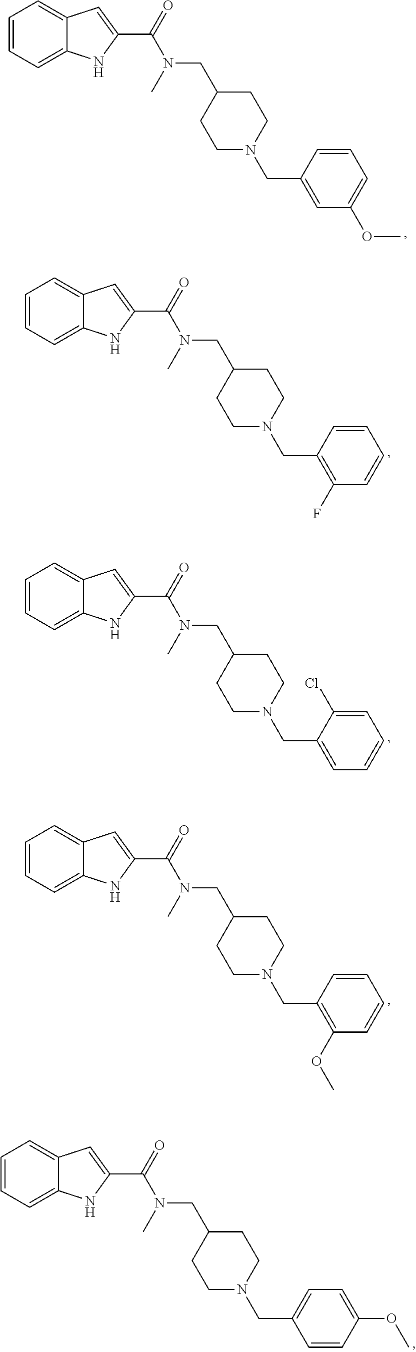 [SMILES: COc1cccc(CN2CCC(CN(C)C(=O)c3cc4ccccc4[nH]3)CC2)c1 ; CN(CC1CCN(Cc2ccccc2F)CC1)C(=O)c1cc2ccccc2[nH]1 ; CN(CC1CCN(Cc2ccccc2Cl)CC1)C(=O)c1cc2ccccc2[nH]1 ; COc1ccccc1CN1CCC(CN(C)C(=O)c2cc3ccccc3[nH]2)CC1 ; COc1ccc(CN2CCC(CN(C)C(=O)c3cc4ccccc4[nH]3)CC2)cc1]

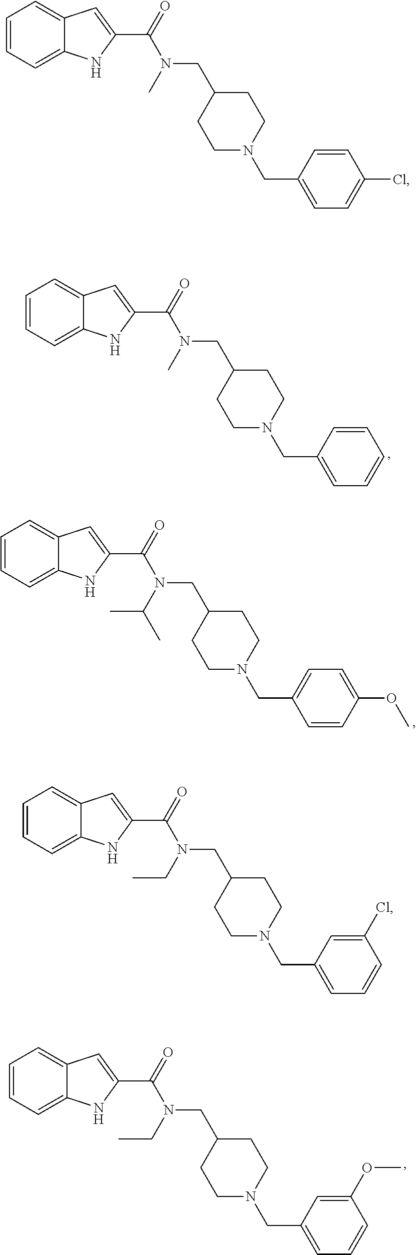 [SMILES: CN(CC1CCN(Cc2ccc(Cl)cc2)CC1)C(=O)c1cc2ccccc2[nH]1 ; CN(CC1CCN(Cc2ccccc2)CC1)C(=O)c1cc2ccccc2[nH]1 ; COc1ccc(CN2CCC(CN(C(=O)c3cc4ccccc4[nH]3)C(C)C)CC2)cc1 ; CCN(CC1CCN(Cc2cccc(Cl)c2)CC1)C(=O)c1cc2ccccc2[nH]1 ; CCN(CC1CCN(Cc2cccc(OC)c2)CC1)C(=O)c1cc2ccccc2[nH]1]

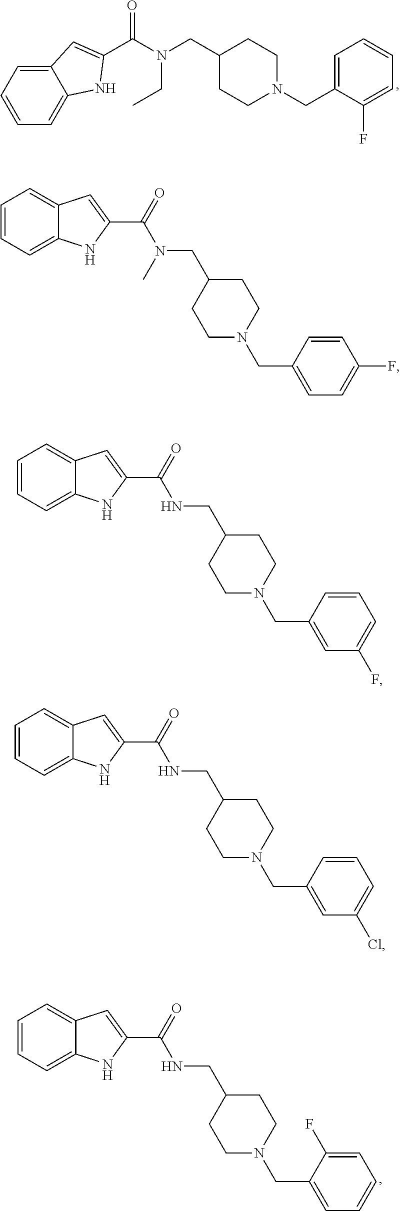 [SMILES: CCN(CC1CCN(Cc2ccccc2F)CC1)C(=O)c1cc2ccccc2[nH]1 ; CN(CC1CCN(Cc2ccc(F)cc2)CC1)C(=O)c1cc2ccccc2[nH]1 ; O=C(NCC1CCN(Cc2cccc(F)c2)CC1)c1cc2ccccc2[nH]1 ; O=C(NCC1CCN(Cc2cccc(Cl)c2)CC1)c1cc2ccccc2[nH]1 ; O=C(NCC1CCN(Cc2ccccc2F)CC1)c1cc2ccccc2[nH]1]

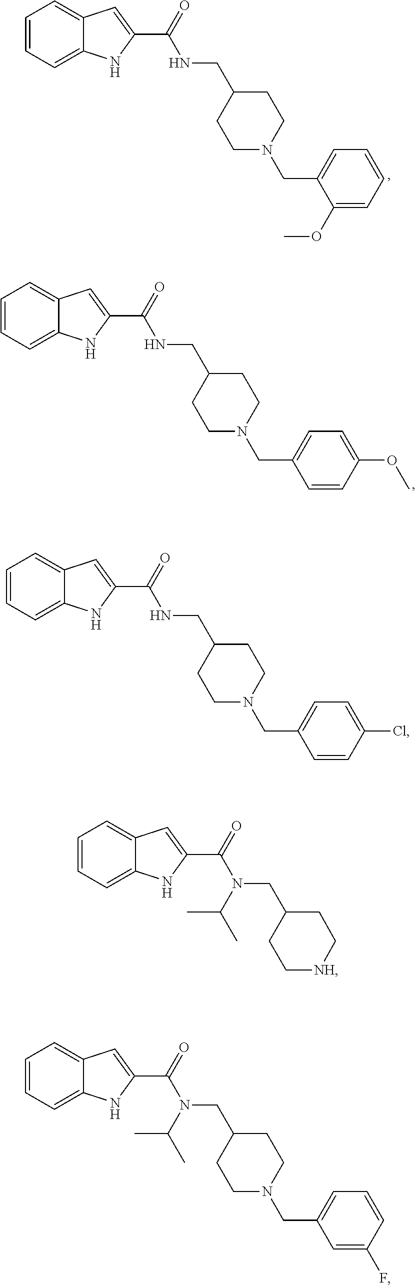 [SMILES: COc1ccccc1CN1CCC(CNC(=O)c2cc3ccccc3[nH]2)CC1 ; COc1ccc(CN2CCC(CNC(=O)c3cc4ccccc4[nH]3)CC2)cc1 ; O=C(NCC1CCN(Cc2ccc(Cl)cc2)CC1)c1cc2ccccc2[nH]1 ; CC(C)N(CC1CCNCC1)C(=O)c1cc2ccccc2[nH]1 ; CC(C)N(CC1CCN(Cc2cccc(F)c2)CC1)C(=O)c1cc2ccccc2[nH]1]

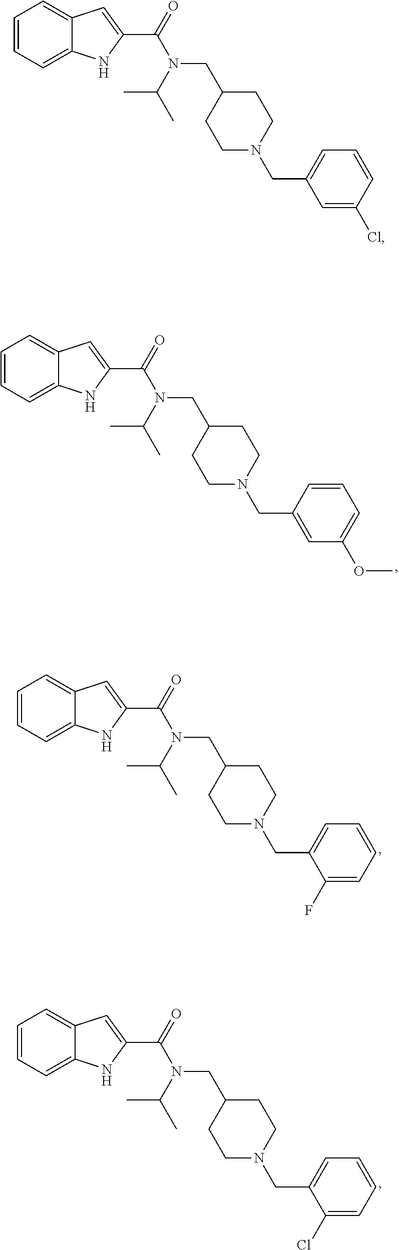 [SMILES: CC(C)N(CC1CCN(Cc2cccc(Cl)c2)CC1)C(=O)c1cc2ccccc2[nH]1 ; COc1cccc(CN2CCC(CN(C(=O)c3cc4ccccc4[nH]3)C(C)C)CC2)c1 ; CC(C)N(CC1CCN(Cc2ccccc2F)CC1)C(=O)c1cc2ccccc2[nH]1 ; CC(C)N(CC1CCN(Cc2ccccc2Cl)CC1)C(=O)c1cc2ccccc2[nH]1]

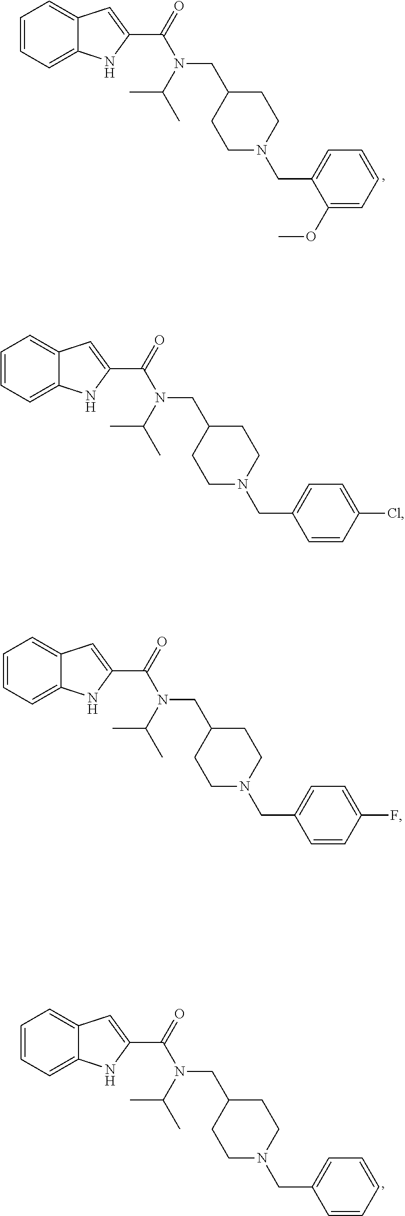 [SMILES: COc1ccccc1CN1CCC(CN(C(=O)c2cc3ccccc3[nH]2)C(C)C)CC1 ; CC(C)N(CC1CCN(Cc2ccc(Cl)cc2)CC1)C(=O)c1cc2ccccc2[nH]1 ; CC(C)N(CC1CCN(Cc2ccc(F)cc2)CC1)C(=O)c1cc2ccccc2[nH]1 ; CC(C)N(CC1CCN(Cc2ccccc2)CC1)C(=O)c1cc2ccccc2[nH]1]

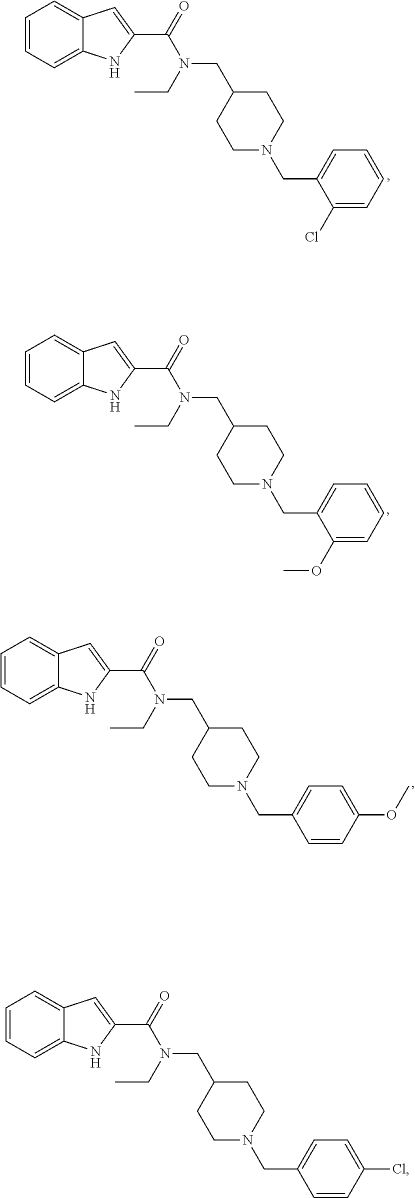 [SMILES: CCN(CC1CCN(Cc2ccccc2Cl)CC1)C(=O)c1cc2ccccc2[nH]1 ; CCN(CC1CCN(Cc2ccccc2OC)CC1)C(=O)c1cc2ccccc2[nH]1 ; CCN(CC1CCN(Cc2ccc(OC)cc2)CC1)C(=O)c1cc2ccccc2[nH]1 ; CCN(CC1CCN(Cc2ccc(Cl)cc2)CC1)C(=O)c1cc2ccccc2[nH]1]

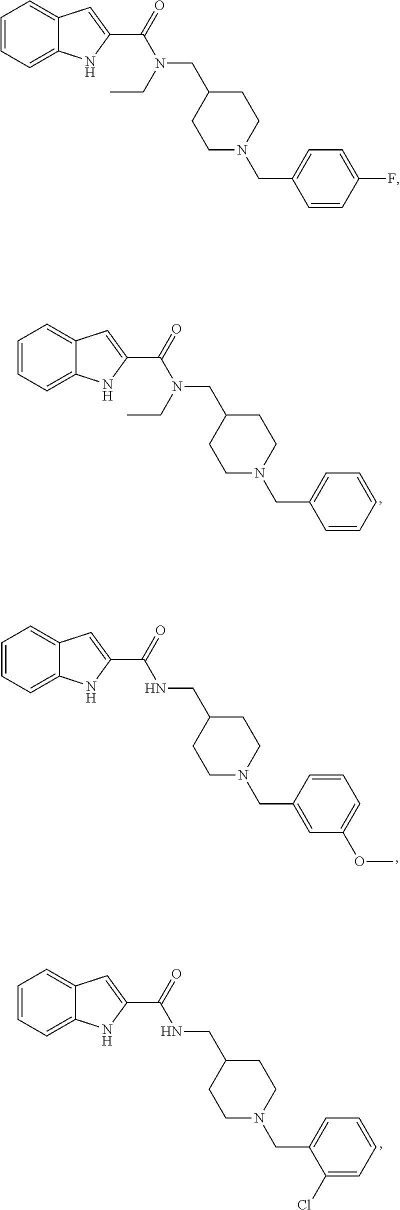 [SMILES: CCN(CC1CCN(Cc2ccc(F)cc2)CC1)C(=O)c1cc2ccccc2[nH]1 ; CCN(CC1CCN(Cc2ccccc2)CC1)C(=O)c1cc2ccccc2[nH]1 ; COc1cccc(CN2CCC(CNC(=O)c3cc4ccccc4[nH]3)CC2)c1 ; O=C(NCC1CCN(Cc2ccccc2Cl)CC1)c1cc2ccccc2[nH]1]

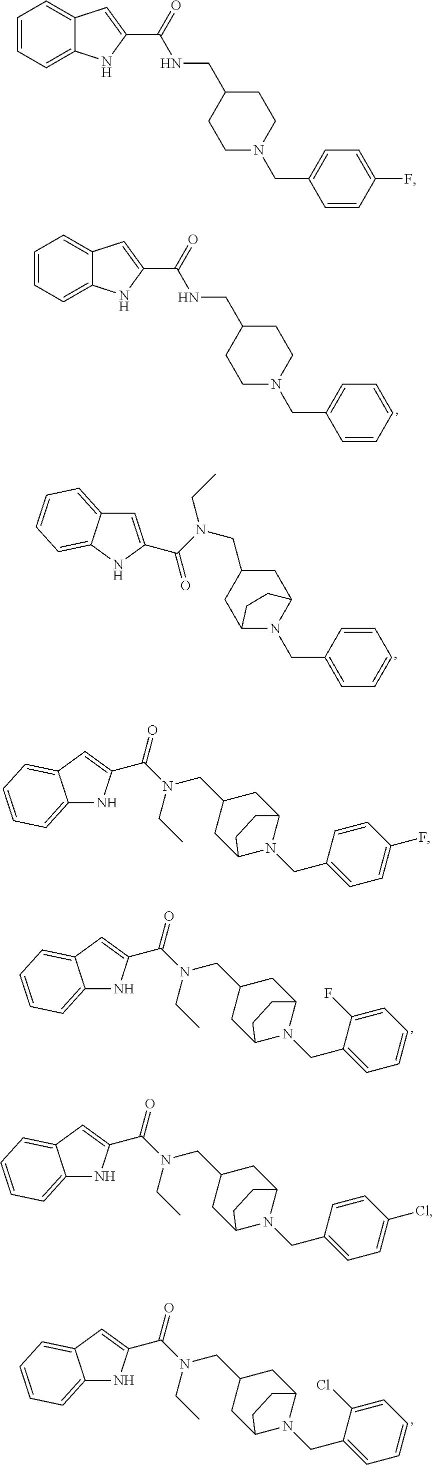

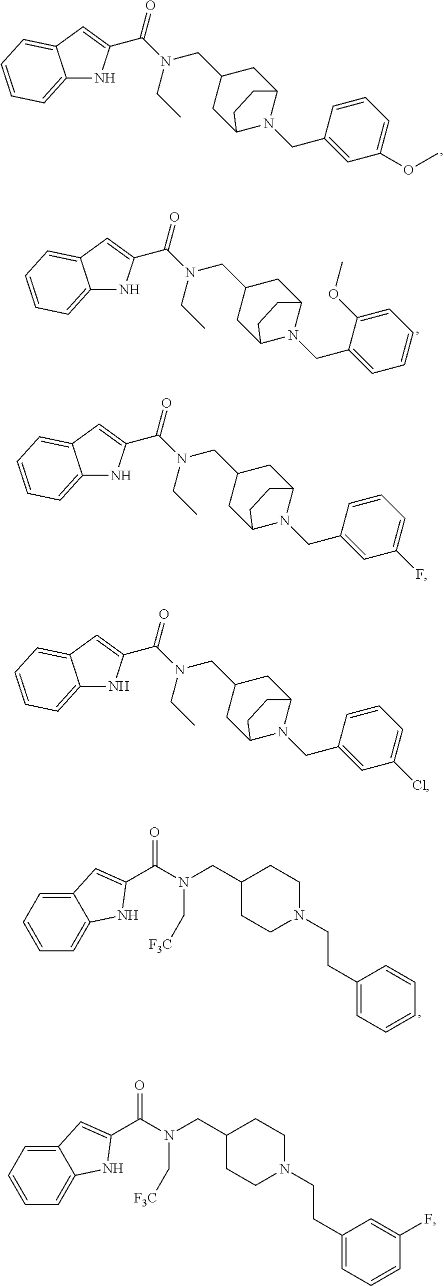 [SMILES: CCN(CC1CC2CCC(C1)N2Cc1cccc(OC)c1)C(=O)c1cc2ccccc2[nH]1 ; CCN(CC1CC2CCC(C1)N2Cc1ccccc1OC)C(=O)c1cc2ccccc2[nH]1 ; CCN(CC1CC2CCC(C1)N2Cc1cccc(F)c1)C(=O)c1cc2ccccc2[nH]1 ; CCN(CC1CC2CCC(C1)N2Cc1cccc(Cl)c1)C(=O)c1cc2ccccc2[nH]1 ; O=C(c1cc2ccccc2[nH]1)N(CC1CCN(CCc2ccccc2)CC1)CC(F)(F)F ; O=C(c1cc2ccccc2[nH]1)N(CC1CCN(CCc2cccc(F)c2)CC1)CC(F)(F)F]

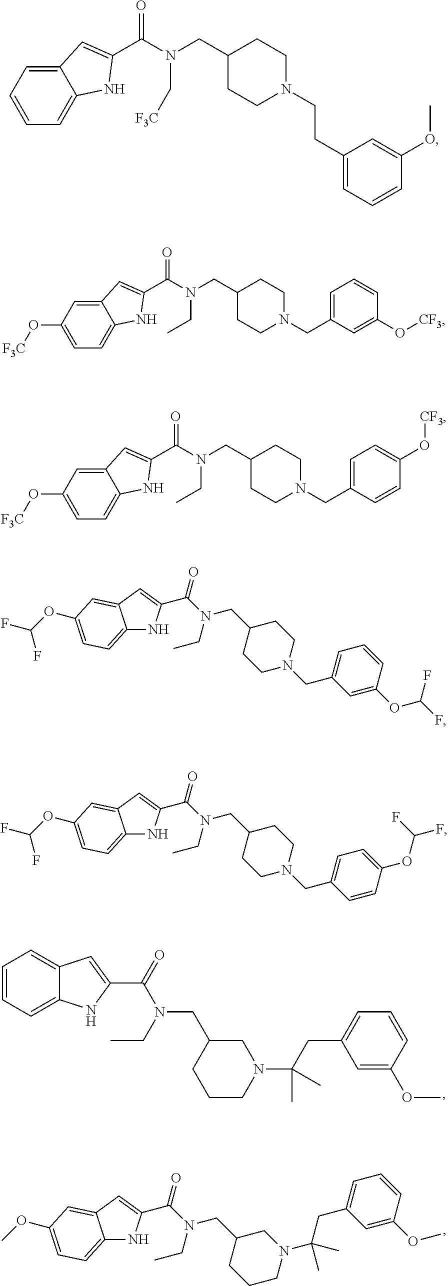

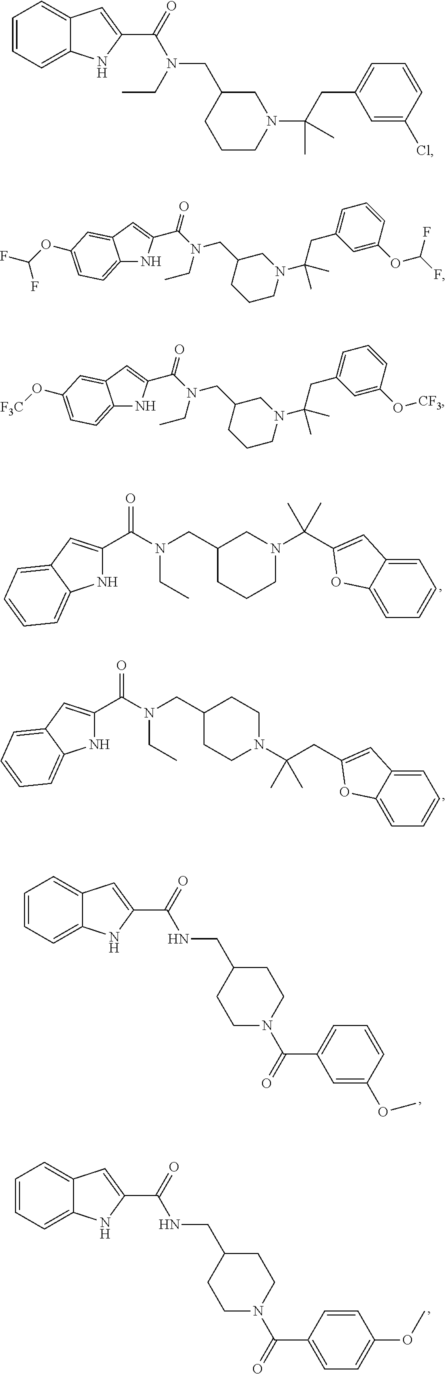

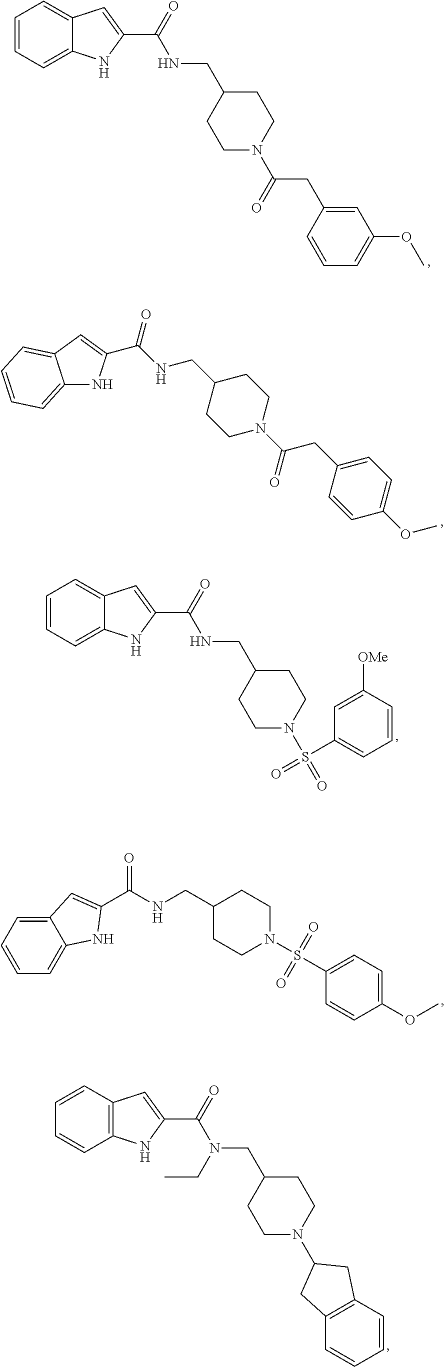 [SMILES: COc1cccc(CC(=O)N2CCC(CNC(=O)c3cc4ccccc4[nH]3)CC2)c1 ; COc1ccc(CC(=O)N2CCC(CNC(=O)c3cc4ccccc4[nH]3)CC2)cc1 ; COc1cccc(S(=O)(=O)N2CCC(CNC(=O)c3cc4ccccc4[nH]3)CC2)c1 ; COc1ccc(S(=O)(=O)N2CCC(CNC(=O)c3cc4ccccc4[nH]3)CC2)cc1 ; CCN(CC1CCN(C2Cc3ccccc3C2)CC1)C(=O)c1cc2ccccc2[nH]1]

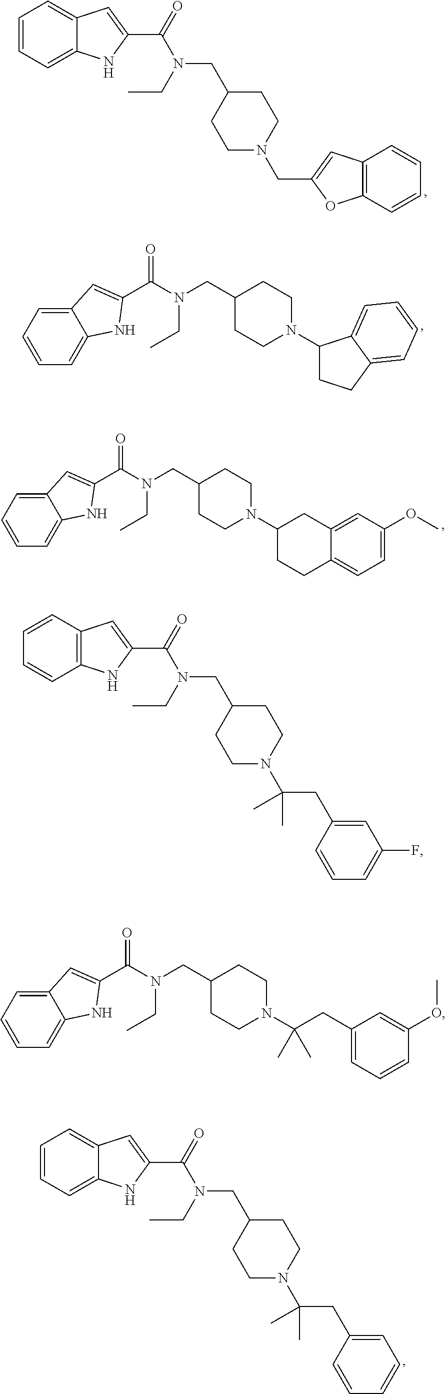 [SMILES: CCN(CC1CCN(Cc2cc3ccccc3o2)CC1)C(=O)c1cc2ccccc2[nH]1 ; CCN(CC1CCN(C2CCc3ccccc32)CC1)C(=O)c1cc2ccccc2[nH]1 ; CCN(CC1CCN(C2CCc3ccc(OC)cc3C2)CC1)C(=O)c1cc2ccccc2[nH]1 ; CCN(CC1CCN(C(C)(C)Cc2cccc(F)c2)CC1)C(=O)c1cc2ccccc2[nH]1 ; CCN(CC1CCN(C(C)(C)Cc2cccc(OC)c2)CC1)C(=O)c1cc2ccccc2[nH]1 ; CCN(CC1CCN(C(C)(C)Cc2ccccc2)CC1)C(=O)c1cc2ccccc2[nH]1]

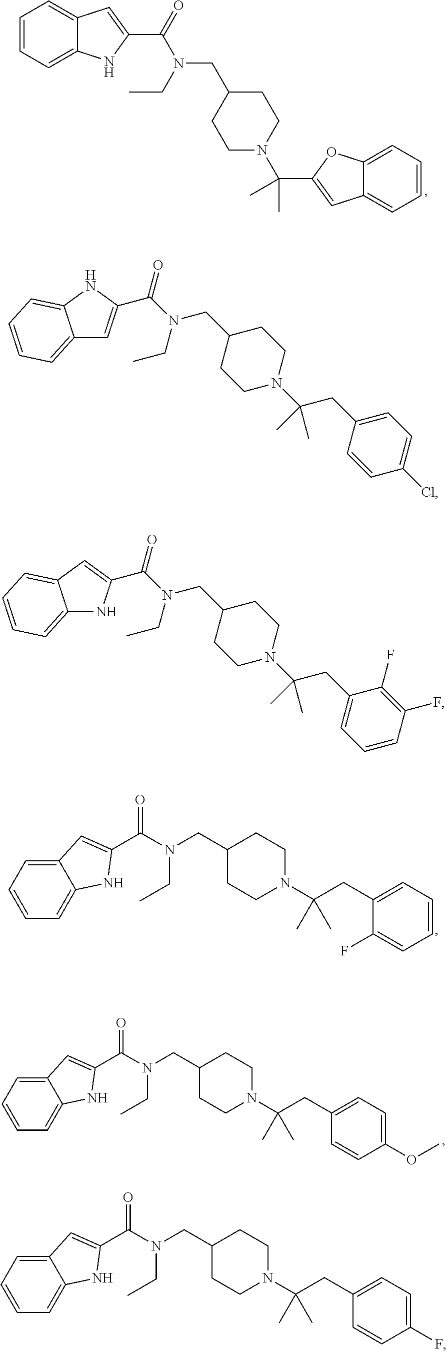 [SMILES: CCN(CC1CCN(C(C)(C)c2cc3ccccc3o2)CC1)C(=O)c1cc2ccccc2[nH]1 ; CCN(CC1CCN(C(C)(C)Cc2ccc(Cl)cc2)CC1)C(=O)c1cc2ccccc2[nH]1 ; CCN(CC1CCN(C(C)(C)Cc2cccc(F)c2F)CC1)C(=O)c1cc2ccccc2[nH]1 ; CCN(CC1CCN(C(C)(C)Cc2ccccc2F)CC1)C(=O)c1cc2ccccc2[nH]1 ; CCN(CC1CCN(C(C)(C)Cc2ccc(OC)cc2)CC1)C(=O)c1cc2ccccc2[nH]1 ; CCN(CC1CCN(C(C)(C)Cc2ccc(F)cc2)CC1)C(=O)c1cc2ccccc2[nH]1]

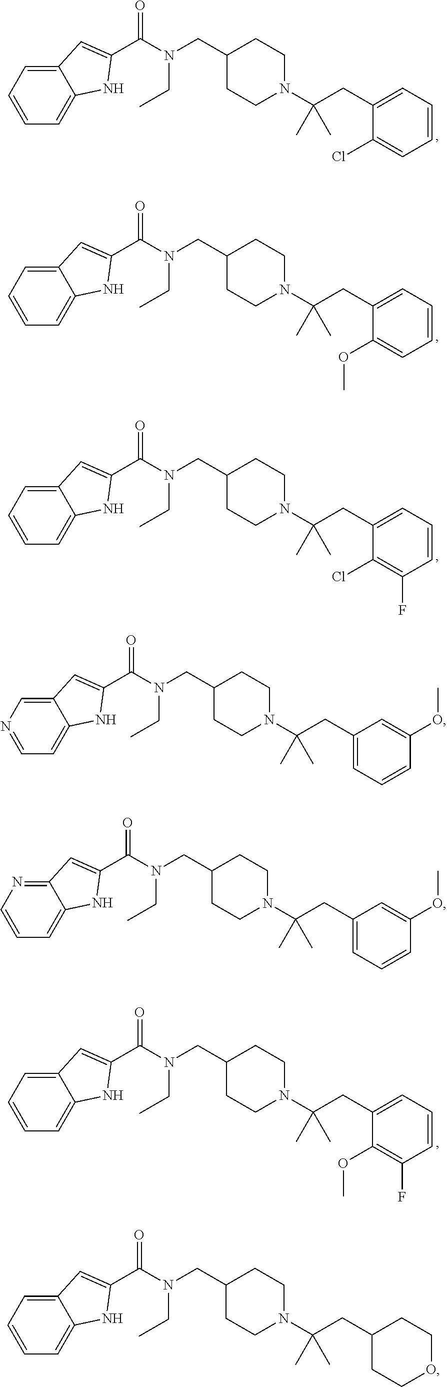

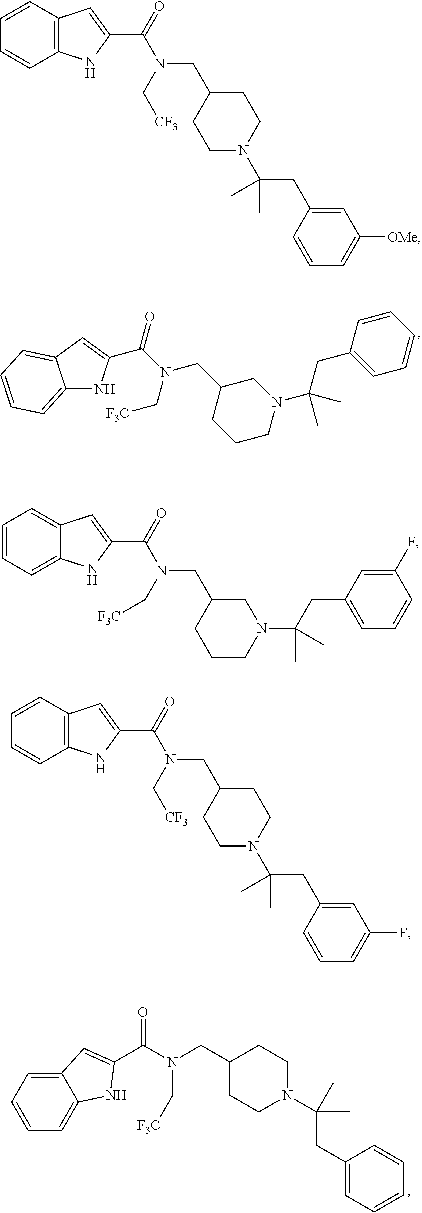 [SMILES: COc1cccc(CC(C)(C)N2CCC(CN(CC(F)(F)F)C(=O)c3cc4ccccc4[nH]3)CC2)c1 ; CC(C)(Cc1ccccc1)N1CCCC(CN(CC(F)(F)F)C(=O)c2cc3ccccc3[nH]2)C1 ; CC(C)(Cc1cccc(F)c1)N1CCCC(CN(CC(F)(F)F)C(=O)c2cc3ccccc3[nH]2)C1 ; CC(C)(Cc1cccc(F)c1)N1CCC(CN(CC(F)(F)F)C(=O)c2cc3ccccc3[nH]2)CC1 ; CC(C)(Cc1ccccc1)N1CCC(CN(CC(F)(F)F)C(=O)c2cc3ccccc3[nH]2)CC1]

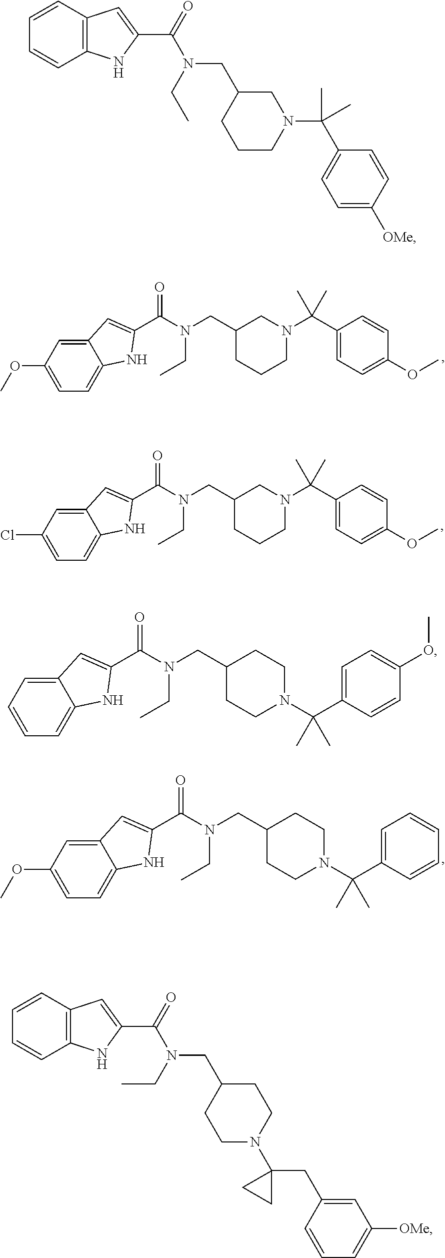

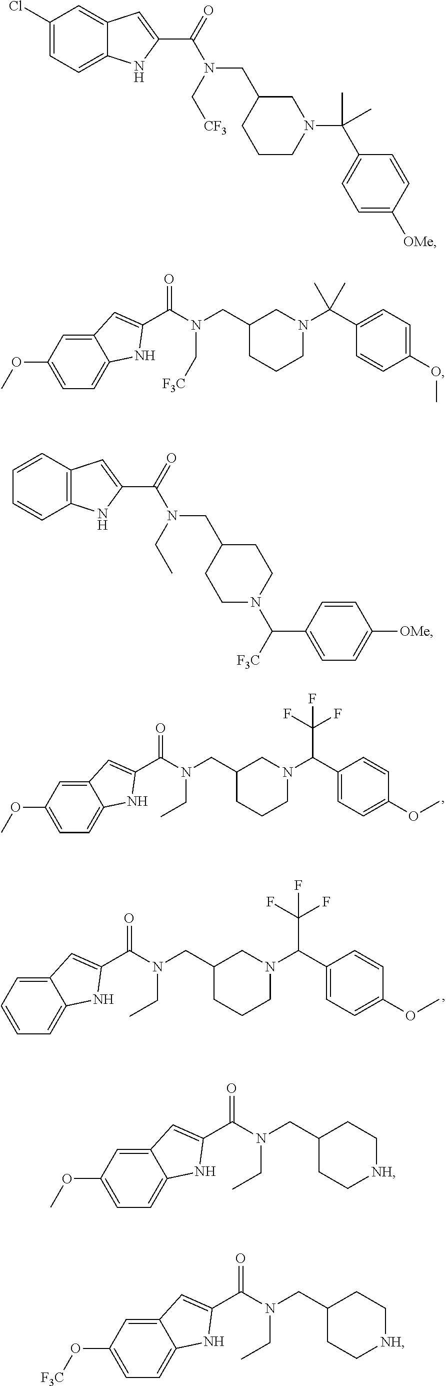

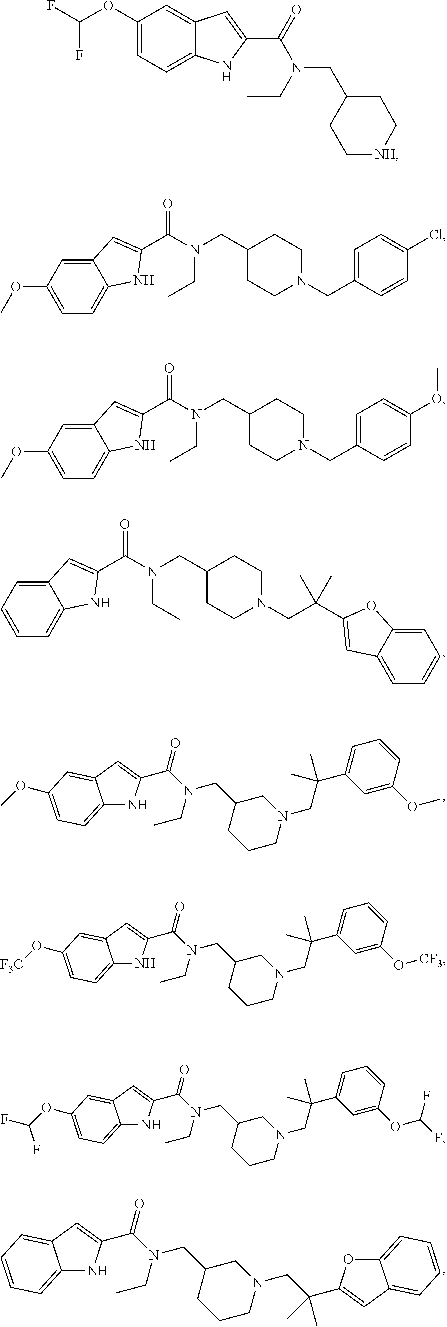

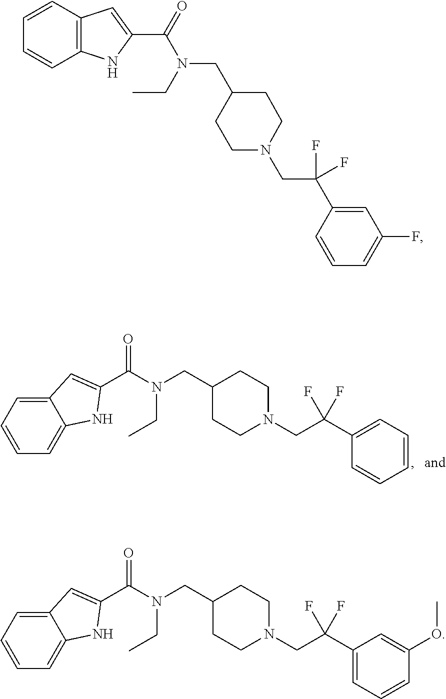 [SMILES: CCN(CC1CCN(CC(F)(F)c2cccc(F)c2)CC1)C(=O)c1cc2ccccc2[nH]1 ; CCN(CC1CCN(CC(F)(F)c2ccccc2)CC1)C(=O)c1cc2ccccc2[nH]1 ; CCN(CC1CCN(CC(F)(F)c2cccc(OC)c2)CC1)C(=O)c1cc2ccccc2[nH]1]

33. A compound of Formula (II): 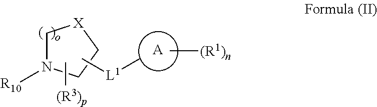
or a pharmaceutically acceptable salt thereof, wherein:
Ring A is cycloalkyl, heterocyclyl, aryl, or heteroaryl;
X is C(R′), C(R′)(R″), N, or NR^{A};
L¹ is a bond, —C₁-C₆ alkyl-, —C₂-C₆ alkenyl-, —C₂-C₆ alkynyl-, —C₁-C₆ heteroalkyl-, —C(O)—, —OC(O)—, —C(O)O—, —OC(O)O—, —C(O)NR^{A}—, —NR^{A}C(O)—, —C(O)NR^{A}—C₁-C₆ alkyl, —C₁-C₆ alkyl-C(O)NR^{A}—, —NR^{A}C(O)—C₁-C₆ alkyl-, —C₁-C₆ alkyl-NR^{A}C(O)—, —C(O)NR^{A}—C₁-C₆ heteroalkyl-, —C₁-C₆ heteroalkyl-C(O)NR^{A}—, —NR^{A}C(O)—C₁-C₆ heteroalkyl-, —C₁-C₆ heteroalkyl-NR^{A}C(O)—, —C₁-C₆ alkyl-C(O)—, —C(O)—C₁-C₆ alkyl, —C₁-C₆ heteroalkyl-C(O)—, —C(O)—C₁-C₆ heteroalkyl, —C(O)—C₁-C₆ alkyl-C(O)NR^{A}—, —S(O)ₓ—, —OS(O)ₓ, —C(O)NR^{A}S(O)ₓ—, —NR^{A}S(O)ₓ—, or —S(O)ₓNR^{A}—, each of which is optionally substituted with 1-5 R⁵;
each R¹ is independently C₁-C₆ alkyl, C₂-C₆ alkenyl, C₂-C₆ alkynyl, C₁-C₆ heteroalkyl, C₁-C₆ haloalkyl, halo, cyano, cycloalkyl, heterocyclyl, aryl, heteroaryl, —OR^{B}, —NR^{A}R^{C}, —NR^{A}C(O)R^{D}, —S(O)ₓR^{E}, —OS(O)ₓR^{E}, —C(O)NR^{A}S(O)ₓR^{E}, —NR^{A}S(O)ₓR^{E}, or —S(O)ₓNR^{A}, each of which is optionally substituted with 1-5 R⁶;
each R³ is independently H, C₁-C₆ alkyl, C₂-C₆ alkenyl, C₂-C₆ alkynyl, C₁-C₆ heteroalkyl, C₁-C₆ haloalkyl, halo, cyano, nitro, cycloalkyl, heterocyclyl, aryl, heteroaryl, —OR^{B}, —NR^{A}R^{C}, —C(O)R^{D}, —C(O)OR^{B}, —C(O)NR^{A}R^{C}, —NR^{A}C(O)R^{D}, —NR^{A}C(O)NR^{B}R^{C}, —SR^{E}, —S(O)ₓR^{E}, —NR^{A}S(O)ₓR^{E}, or —S(O)ₓNR^{A}R^{C}, each of which is optionally substituted with 1-5 R⁷; or
or two R³, taken together with the atoms to which they are attached, form a ring (e.g., a 5-7 membered ring), optionally substituted with 1-5 R⁷;
each of R′ and R″ is independently H, C₁-C₆ alkyl, C₁-C₆ heteroalkyl, C₁-C₆ haloalkyl, halo, cyano, cycloalkyl, or heterocyclyl, each of which is optionally substituted with 1-5 R⁷;
each of R⁵, R⁶, and R⁷ is independently C₁-C₆ alkyl, C₂-C₆ alkenyl, C₂-C₆ alkynyl, C₁-C₆ heteroalkyl, C₁-C₆ haloalkyl, halo, cyano, cycloalkyl, heterocyclyl, aryl, heteroaryl, —OR^{B}, —C(O)R^{D}, —C(O)OR^{B}, —C(O)NR^{A}R^{C}, or —SR^{E}, each of which is optionally substituted with 1-5 R⁸;
each R¹⁰ is independently H, C₁-C₆ alkyl, C₂-C₆ alkenyl, C₂-C₆ alkynyl, C₁-C₆ heteroalkyl, C₁-C₆ haloalkyl, cycloalkyl, heterocyclyl, or —C(O)R^{D}, each of which is optionally substituted with 1-5 R⁸;
each R^{A}, R^{B}, R^{C}, R^{D}, or R^{E} is independently H, C₁-C₆ alkyl, C₁-C₆ heteroalkyl, C₁-C₆ haloalkyl, cycloalkyl, heterocyclyl, aryl, heteroaryl, arylalkyl, heteroarylalkyl, cycloalkylalkyl, or heterocycloalkyl, each of which is optionally substituted with 1-4 R⁸;
or R^{A} and R^{C}, together with the atoms to which each is attached, form a heterocyclyl ring optionally substituted with 1-4 R⁸;
each R⁸ is independently C₁-C₆ alkyl, C₂-C₆ alkenyl, C₂-C₆ alkynyl, C₁-C₆ heteroalkyl, C₁-C₆ haloalkyl, cycloalkyl, heterocyclyl, aryl, heteroaryl, halo, cyano, or nitro, each of which is optionally substituted with 1-5 R⁹;
each R⁹ is C₁-C₆ alkyl, halo, hydroxy, cycloalkyl, alkoxy, keto, cyano, or nitro;
n is 0, 1, 2, 3, 4, 5, or 6;
o is 1, 2, or 3;
p is 0, 1, 2, 3 or 4; and
x is 0, 1, or 2;
wherein when L¹ is connected to X, X is C(R′) or N.

34. The compound of claim 33, wherein Ring A is aryl.

35. The compound of claim 33, wherein Ring A is phenyl.

36. The compound of claim 33, wherein Ring A is heteroaryl.

37. The compound of claim 36, wherein Ring A is a monocyclic heteroaryl or bicyclic heteroaryl.

38. The compound of claim 36, wherein Ring A is indolyl, indolinyl, indazolyl, benzofuranyl, benzoimidazolyl, benzooxazolyl, or benzothiazolyl.

39. The compound of claim 36, wherein Ring A is indolyl.

40. The compound of any-one of claim 36, wherein Ring A is pyrrolyl, furanyl, or pyridyl.

41. The compound of claim 33, wherein n is 0.

42. The compound of claim 33, wherein n is 1, 2, or 3, and R¹ is C₁-C₆ alkyl, halo, cyano, or —OR^{B}.

43. The compound of claim 33, wherein X is C(R′)(R″).

44. The compound of claim 33, wherein each of R′ and R″ is independently H.

45. The compound of claim 33, wherein X is NR^{A}, and R^{A} is H.

46. The compound of claim 33, wherein L¹ is a bond, C₁-C₆ alkyl, C₁-C₆ heteroalkyl, —C(O)—, —C(O)NR^{A}—, —NR^{A}C(O)—, —C(O)NR^{A}—C₁-C₆ alkyl, —NR^{A}C(O)—C₁-C₆ alkyl, —NR^{A}C(O)—C₁-C₆ heteroalkyl, —C(O)—C₁-C₆ alkyl, C₁-C₆ alkyl-C(O)—, C₁-C₆ alkyl-NR^{A}C(O)—, —S(O)ₓ—, —OS(O)ₓ, —C(O)NR^{A}S(O)ₓ—, —NR^{A}S(O)ₓ—, or —S(O)ₓNR^{A}—, each of which is optionally substituted with 1-5 R⁵.

47. The compound of claim 33, wherein L¹ is C₁-C₆ alkyl or C₁-C₆ alkyl-NR^{A}C(O)—.

48. The compound of claim 33, wherein L¹ is C₁-C₆ alkyl-NR^{A}C(O)— and R^{A} is H, C₁-C₆ alkyl, C₁-C₆ heteroalkyl, C₁-C₆ haloalkyl, cycloalkyl, aryl, cycloalkylalkyl, or arylalkyl.

49. The compound of claim 33, wherein p is 0.

50. The compound of claim 33, wherein p is 2 and each R³ is independently C₁-C₆ alkyl (e.g., methyl or ethyl), wherein both R³ is joined together to form a 6- or 7-membered ring.

51. The compound of any one of claim 33, wherein o is 2.

52. The compound of claim 33, wherein the compound of Formula (I) is selected from

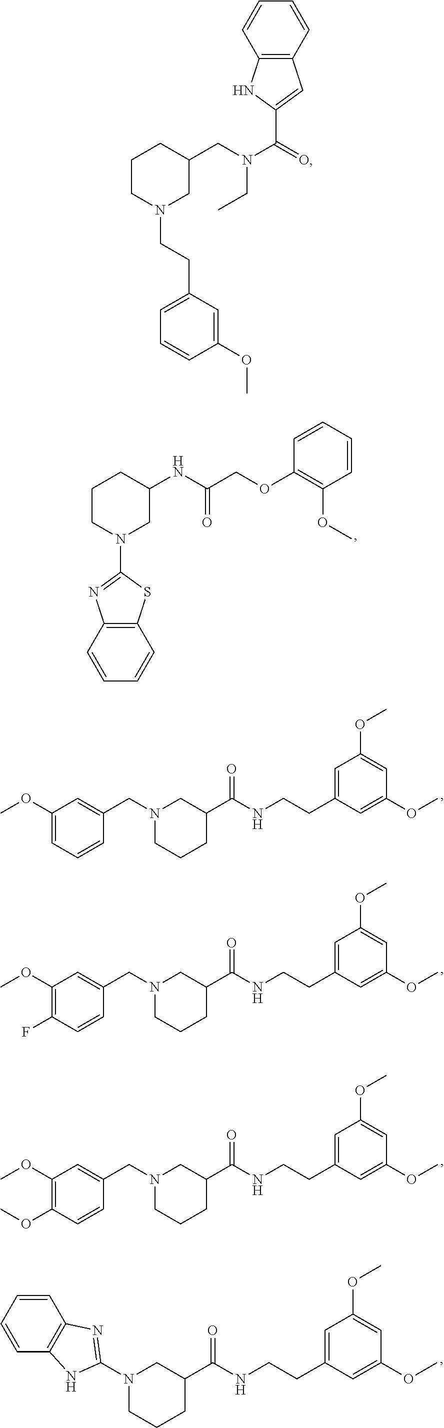

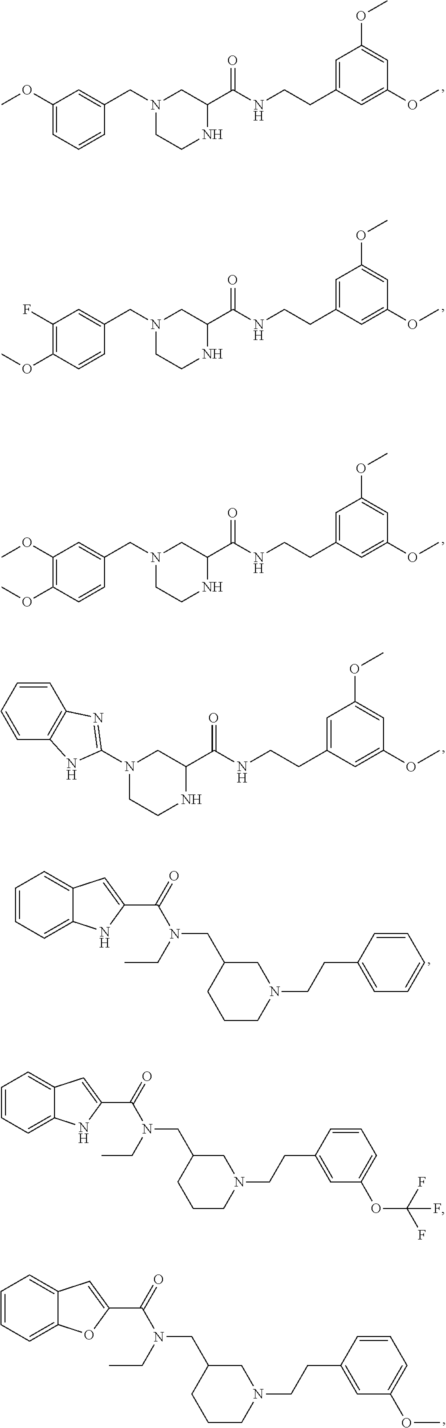

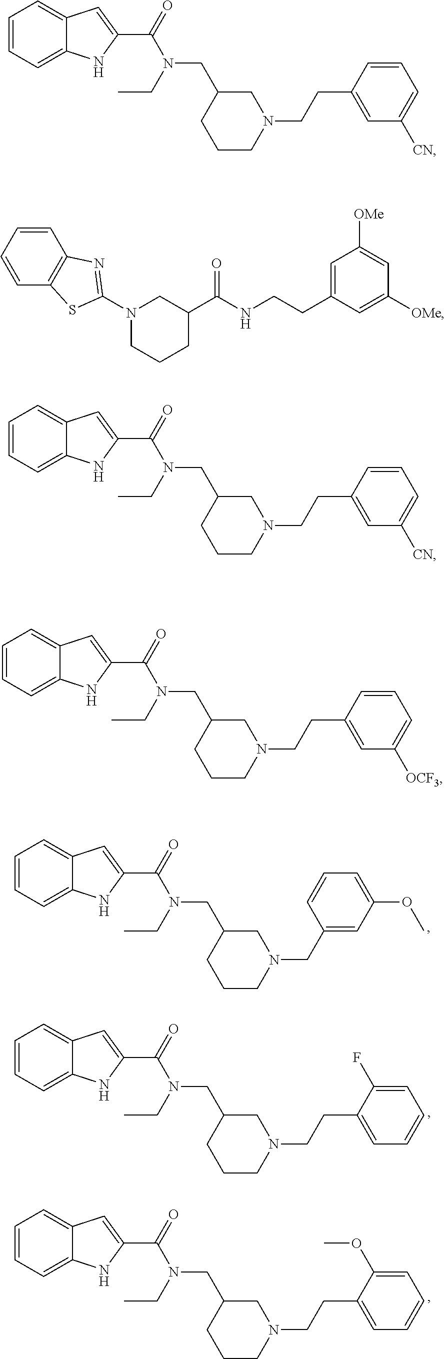

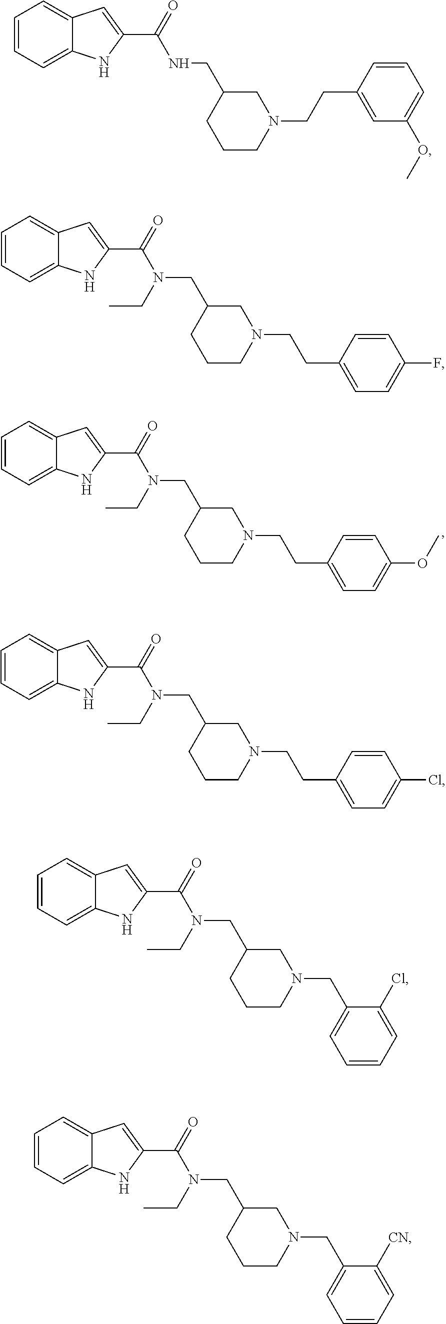 [SMILES: COc1cccc(CCN2CCCC(CNC(=O)c3cc4ccccc4[nH]3)C2)c1 ; CCN(CC1CCCN(CCc2ccc(F)cc2)C1)C(=O)c1cc2ccccc2[nH]1 ; CCN(CC1CCCN(CCc2ccc(OC)cc2)C1)C(=O)c1cc2ccccc2[nH]1 ; CCN(CC1CCCN(CCc2ccc(Cl)cc2)C1)C(=O)c1cc2ccccc2[nH]1 ; CCN(CC1CCCN(Cc2ccccc2Cl)C1)C(=O)c1cc2ccccc2[nH]1 ; CCN(CC1CCCN(Cc2ccccc2C#N)C1)C(=O)c1cc2ccccc2[nH]1]

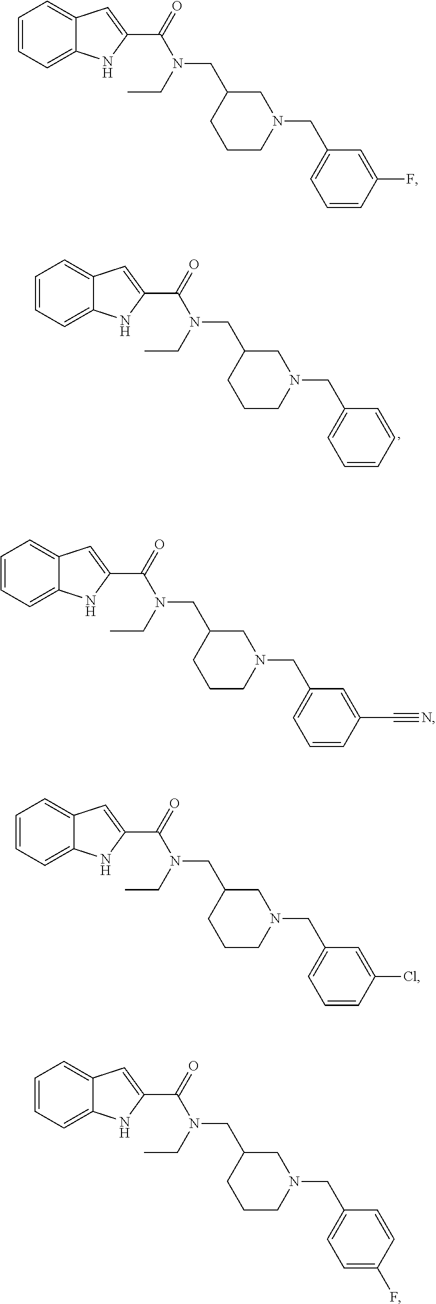 [SMILES: CCN(CC1CCCN(Cc2cccc(F)c2)C1)C(=O)c1cc2ccccc2[nH]1 ; CCN(CC1CCCN(Cc2ccccc2)C1)C(=O)c1cc2ccccc2[nH]1 ; CCN(CC1CCCN(Cc2cccc(C#N)c2)C1)C(=O)c1cc2ccccc2[nH]1 ; CCN(CC1CCCN(Cc2cccc(Cl)c2)C1)C(=O)c1cc2ccccc2[nH]1 ; CCN(CC1CCCN(Cc2ccc(F)cc2)C1)C(=O)c1cc2ccccc2[nH]1]

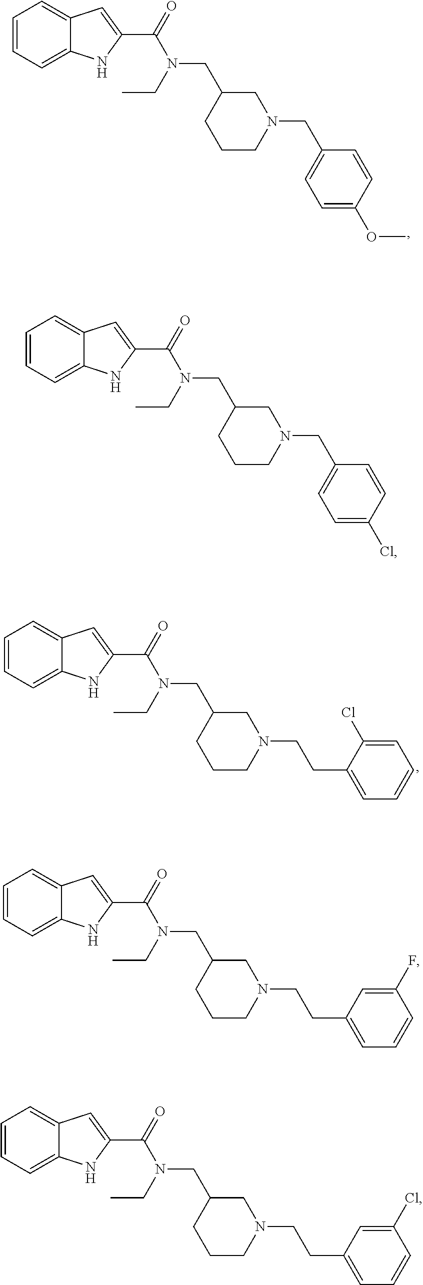 [SMILES: CCN(CC1CCCN(Cc2ccc(OC)cc2)C1)C(=O)c1cc2ccccc2[nH]1 ; CCN(CC1CCCN(Cc2ccc(Cl)cc2)C1)C(=O)c1cc2ccccc2[nH]1 ; CCN(CC1CCCN(CCc2ccccc2Cl)C1)C(=O)c1cc2ccccc2[nH]1 ; CCN(CC1CCCN(CCc2cccc(F)c2)C1)C(=O)c1cc2ccccc2[nH]1 ; CCN(CC1CCCN(CCc2cccc(Cl)c2)C1)C(=O)c1cc2ccccc2[nH]1]

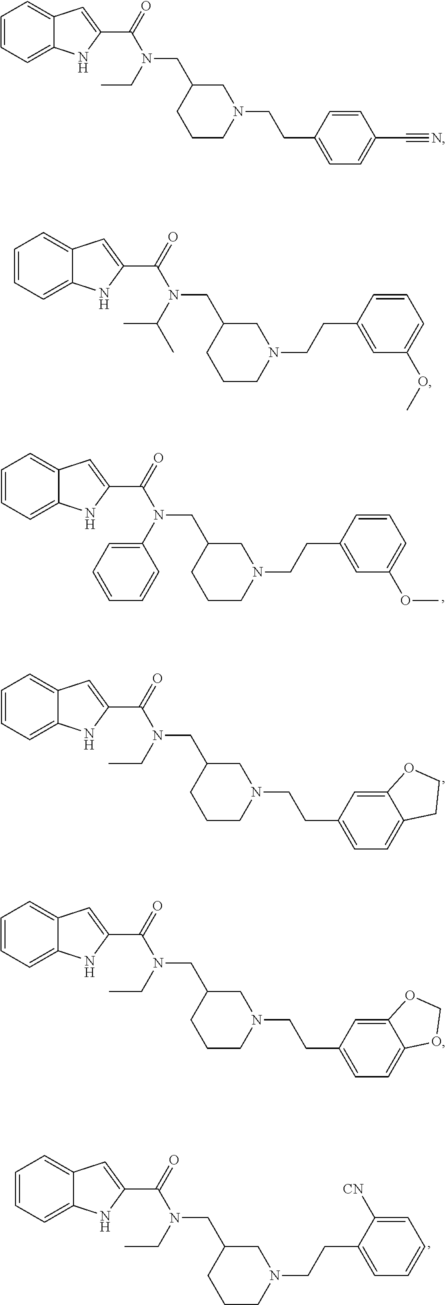

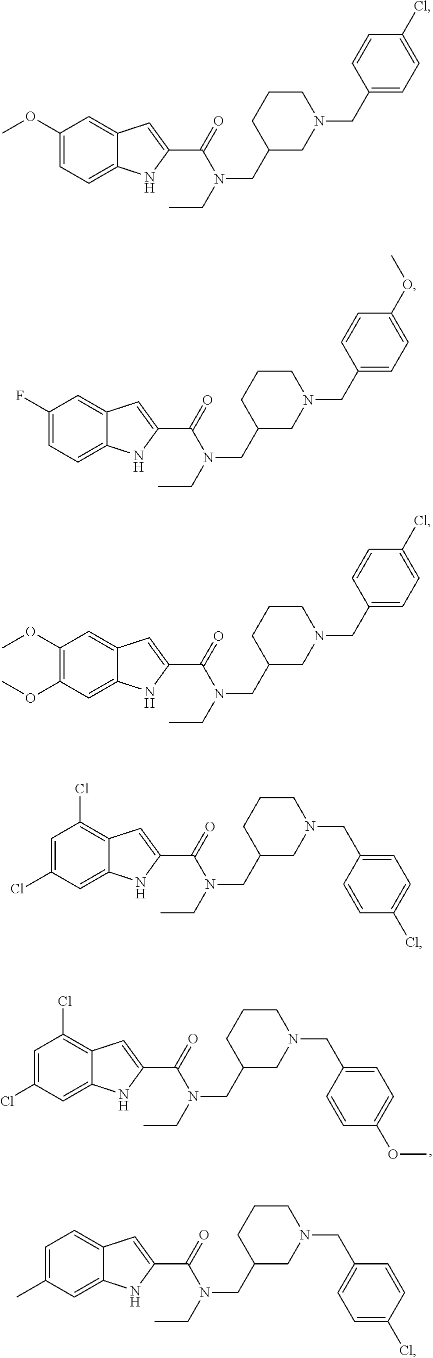

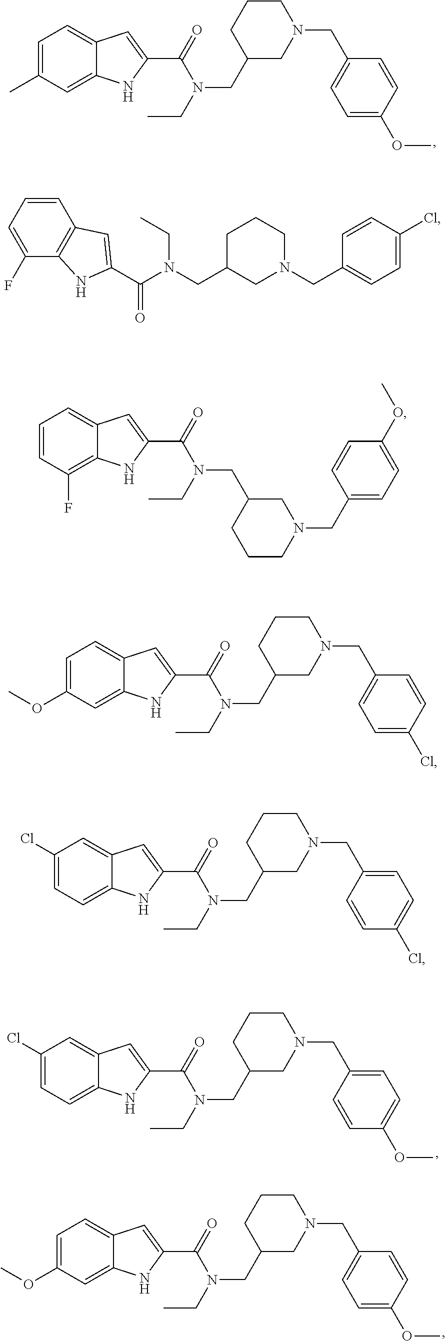

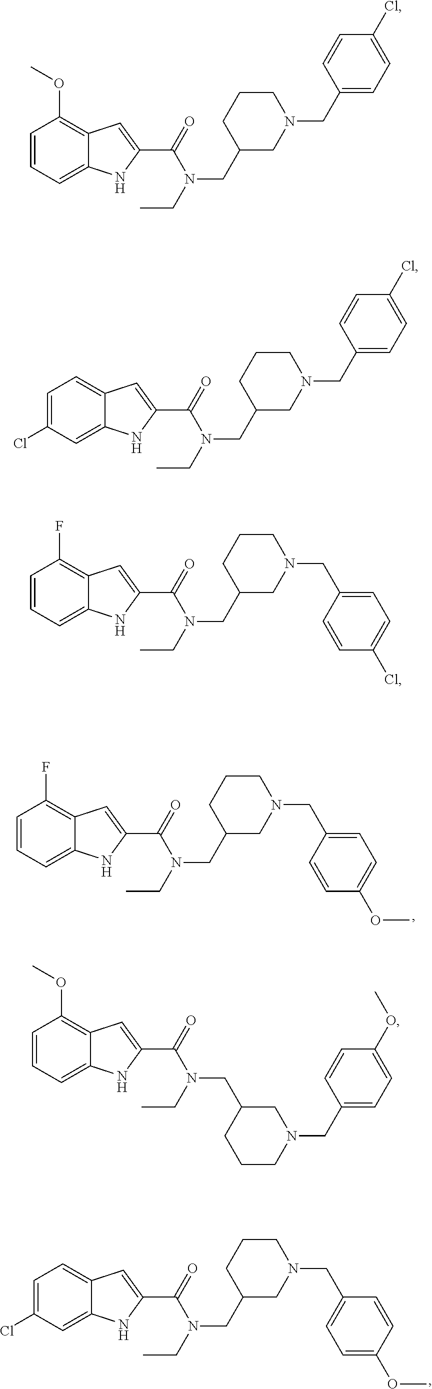 [SMILES: CCN(CC1CCCN(Cc2ccc(Cl)cc2)C1)C(=O)c1cc2c(OC)cccc2[nH]1 ; CCN(CC1CCCN(Cc2ccc(Cl)cc2)C1)C(=O)c1cc2ccc(Cl)cc2[nH]1 ; CCN(CC1CCCN(Cc2ccc(Cl)cc2)C1)C(=O)c1cc2c(F)cccc2[nH]1 ; CCN(CC1CCCN(Cc2ccc(OC)cc2)C1)C(=O)c1cc2c(F)cccc2[nH]1 ; CCN(CC1CCCN(Cc2ccc(OC)cc2)C1)C(=O)c1cc2c(OC)cccc2[nH]1 ; CCN(CC1CCCN(Cc2ccc(OC)cc2)C1)C(=O)c1cc2ccc(Cl)cc2[nH]1]

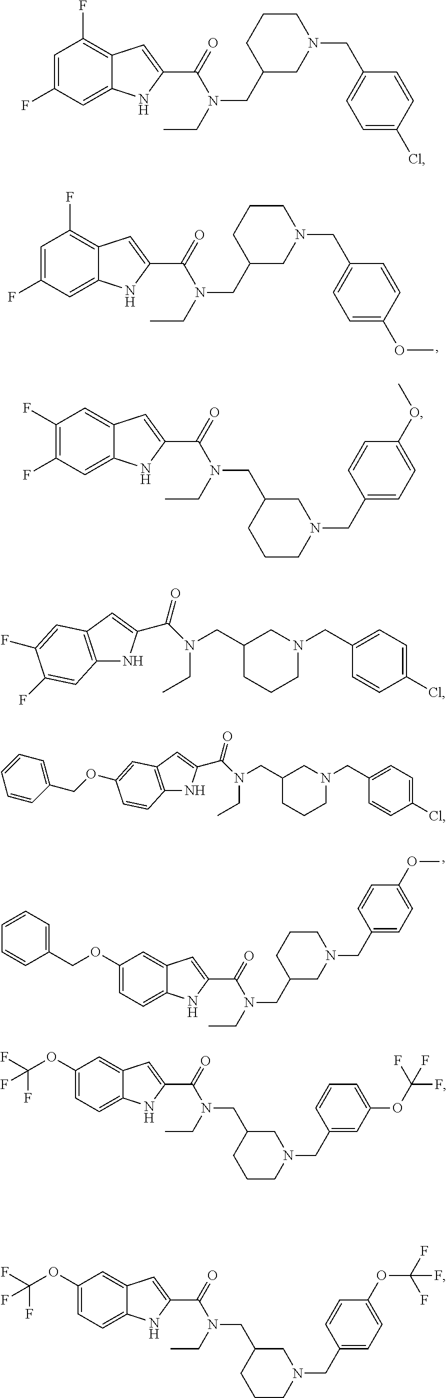

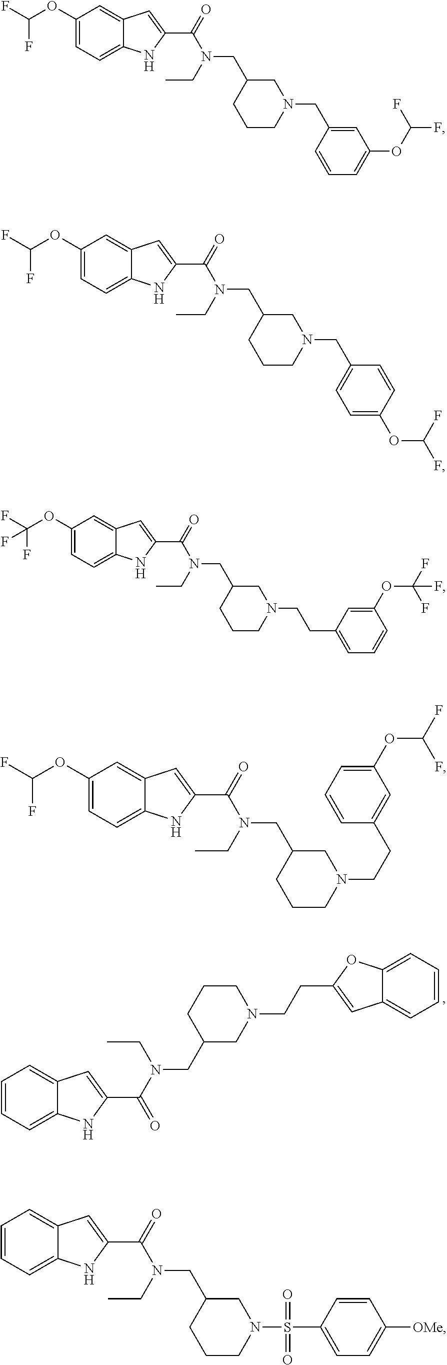 [SMILES: CCN(CC1CCCN(Cc2cccc(OC(F)F)c2)C1)C(=O)c1cc2cc(OC(F)F)ccc2[nH]1 ; CCN(CC1CCCN(Cc2ccc(OC(F)F)cc2)C1)C(=O)c1cc2cc(OC(F)F)ccc2[nH]1 ; CCN(CC1CCCN(CCc2cccc(OC(F)(F)F)c2)C1)C(=O)c1cc2cc(OC(F)(F)F)ccc2[nH]1 ; CCN(CC1CCCN(CCc2cccc(OC(F)F)c2)C1)C(=O)c1cc2cc(OC(F)F)ccc2[nH]1 ; CCN(CC1CCCN(CCc2cc3ccccc3o2)C1)C(=O)c1cc2ccccc2[nH]1 ; CCN(CC1CCCN(S(=O)(=O)c2ccc(OC)cc2)C1)C(=O)c1cc2ccccc2[nH]1]

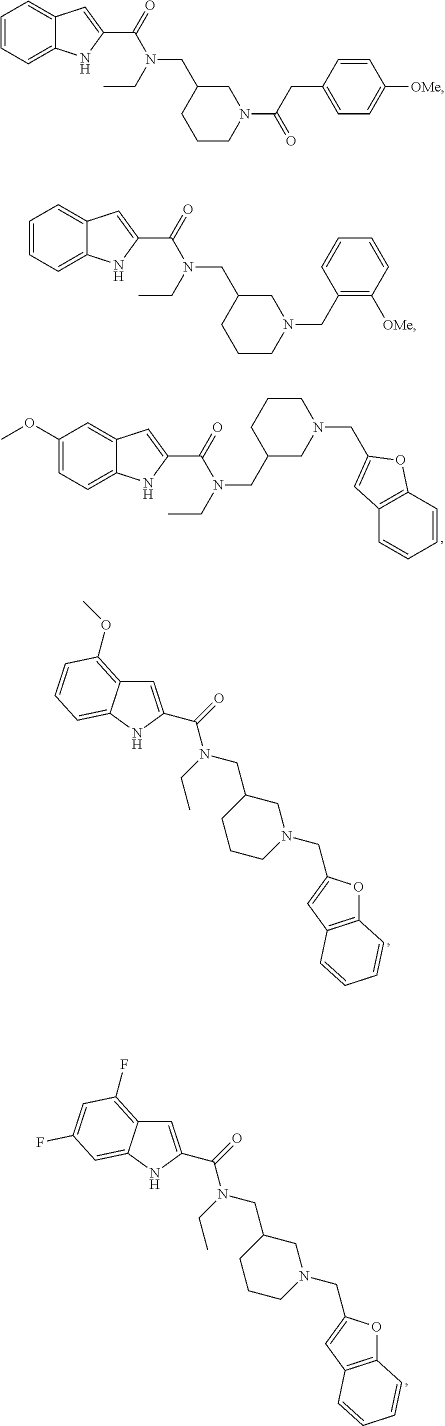

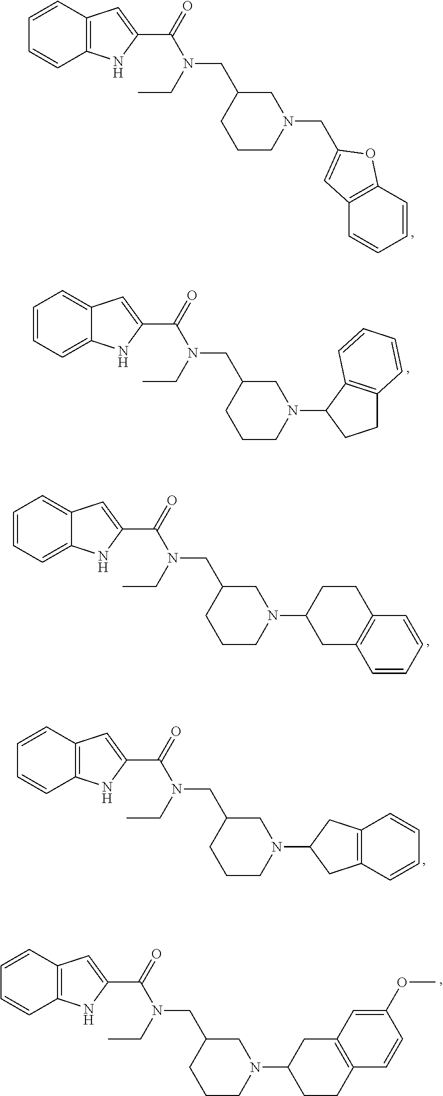 [SMILES: CCN(CC1CCCN(Cc2cc3ccccc3o2)C1)C(=O)c1cc2ccccc2[nH]1 ; CCN(CC1CCCN(C2CCc3ccccc32)C1)C(=O)c1cc2ccccc2[nH]1 ; CCN(CC1CCCN(C2CCc3ccccc3C2)C1)C(=O)c1cc2ccccc2[nH]1 ; CCN(CC1CCCN(C2Cc3ccccc3C2)C1)C(=O)c1cc2ccccc2[nH]1 ; CCN(CC1CCCN(C2CCc3ccc(OC)cc3C2)C1)C(=O)c1cc2ccccc2[nH]1]

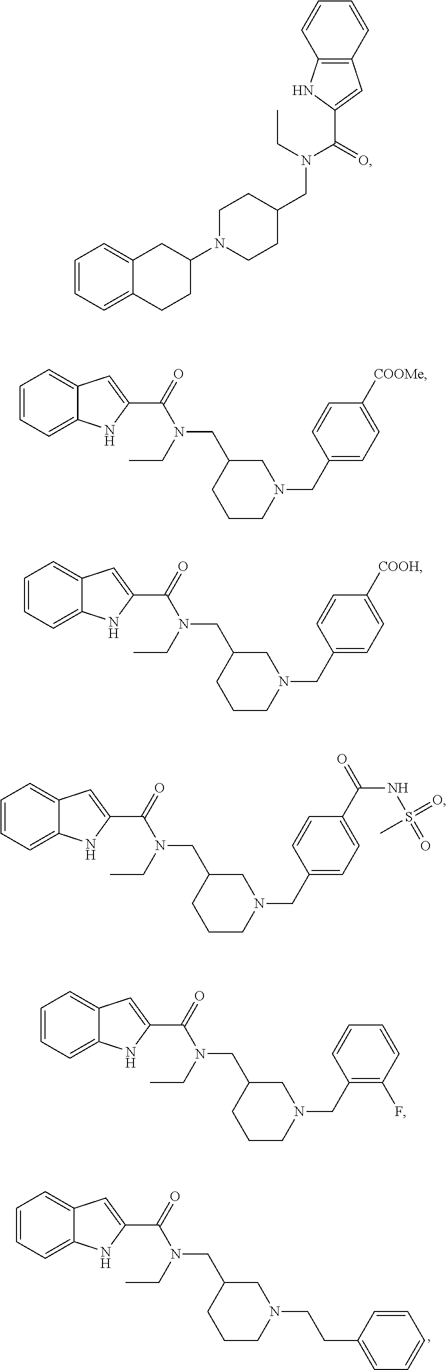 [SMILES: CCN(CC1CCN(C2CCc3ccccc3C2)CC1)C(=O)c1cc2ccccc2[nH]1 ; CCN(CC1CCCN(Cc2ccc(C(=O)OC)cc2)C1)C(=O)c1cc2ccccc2[nH]1 ; CCN(CC1CCCN(Cc2ccc(C(=O)O)cc2)C1)C(=O)c1cc2ccccc2[nH]1 ; CCN(CC1CCCN(Cc2ccc(C(=O)NS(C)(=O)=O)cc2)C1)C(=O)c1cc2ccccc2[nH]1 ; CCN(CC1CCCN(Cc2ccccc2F)C1)C(=O)c1cc2ccccc2[nH]1 ; CCN(CC1CCCN(CCc2ccccc2)C1)C(=O)c1cc2ccccc2[nH]1]

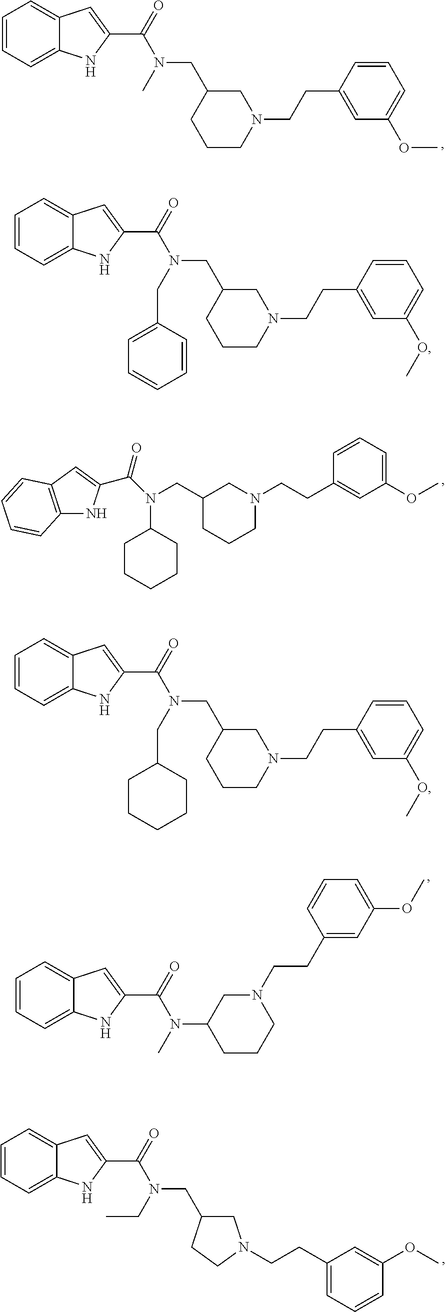 [SMILES: COc1cccc(CCN2CCCC(CN(C)C(=O)c3cc4ccccc4[nH]3)C2)c1 ; COc1cccc(CCN2CCCC(CN(Cc3ccccc3)C(=O)c3cc4ccccc4[nH]3)C2)c1 ; COc1cccc(CCN2CCCC(CN(C(=O)c3cc4ccccc4[nH]3)C3CCCCC3)C2)c1 ; COc1cccc(CCN2CCCC(CN(CC3CCCCC3)C(=O)c3cc4ccccc4[nH]3)C2)c1 ; COc1cccc(CCN2CCCC(N(C)C(=O)c3cc4ccccc4[nH]3)C2)c1 ; CCN(CC1CCN(CCc2cccc(OC)c2)C1)C(=O)c1cc2ccccc2[nH]1]

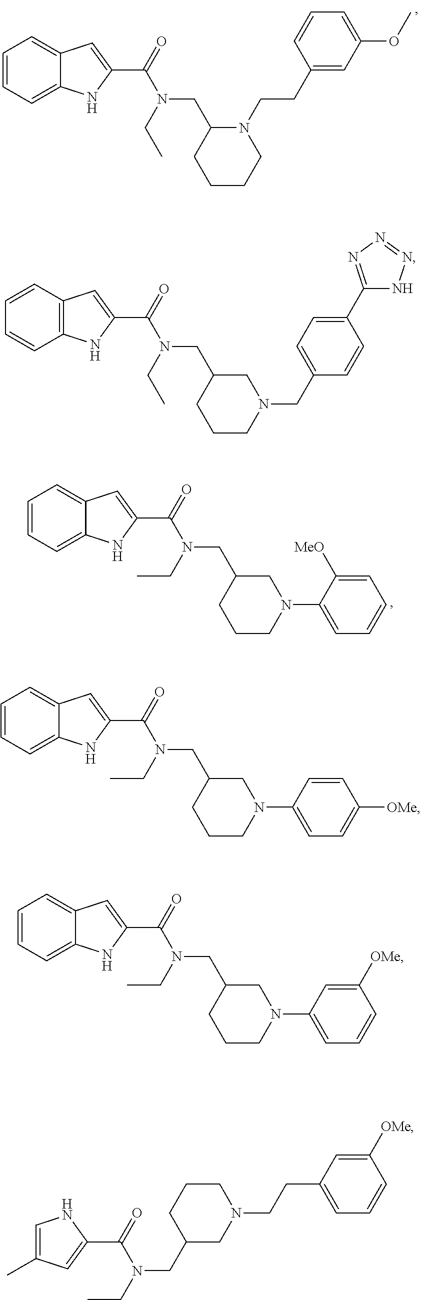

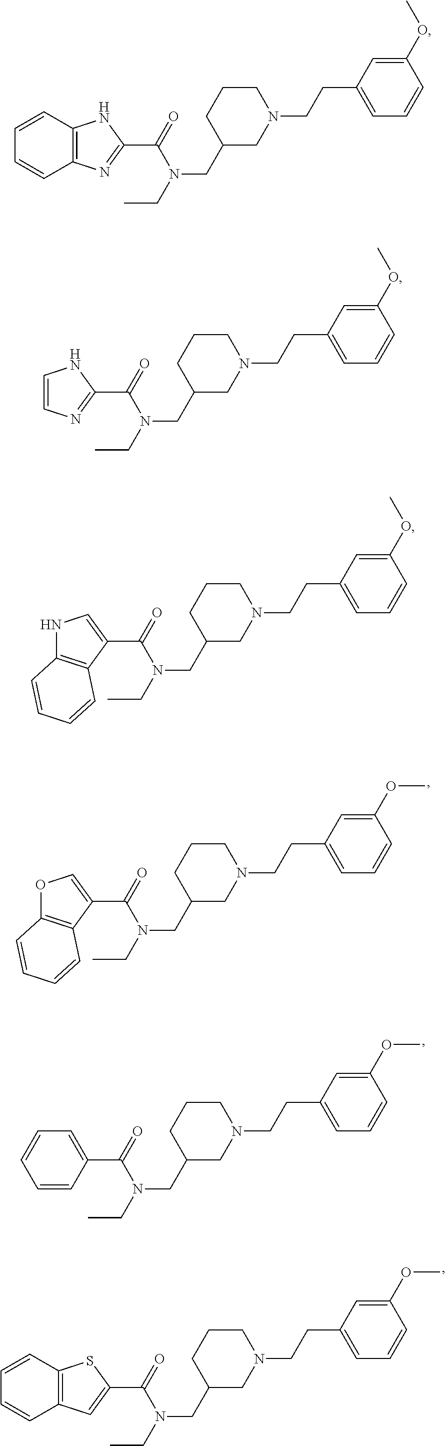 [SMILES: CCN(CC1CCCN(CCc2cccc(OC)c2)C1)C(=O)c1nc2ccccc2[nH]1 ; CCN(CC1CCCN(CCc2cccc(OC)c2)C1)C(=O)c1ncc[nH]1 ; CCN(CC1CCCN(CCc2cccc(OC)c2)C1)C(=O)c1c[nH]c2ccccc12 ; CCN(CC1CCCN(CCc2cccc(OC)c2)C1)C(=O)c1coc2ccccc12 ; CCN(CC1CCCN(CCc2cccc(OC)c2)C1)C(=O)c1ccccc1 ; CCN(CC1CCCN(CCc2cccc(OC)c2)C1)C(=O)c1cc2ccccc2s1]

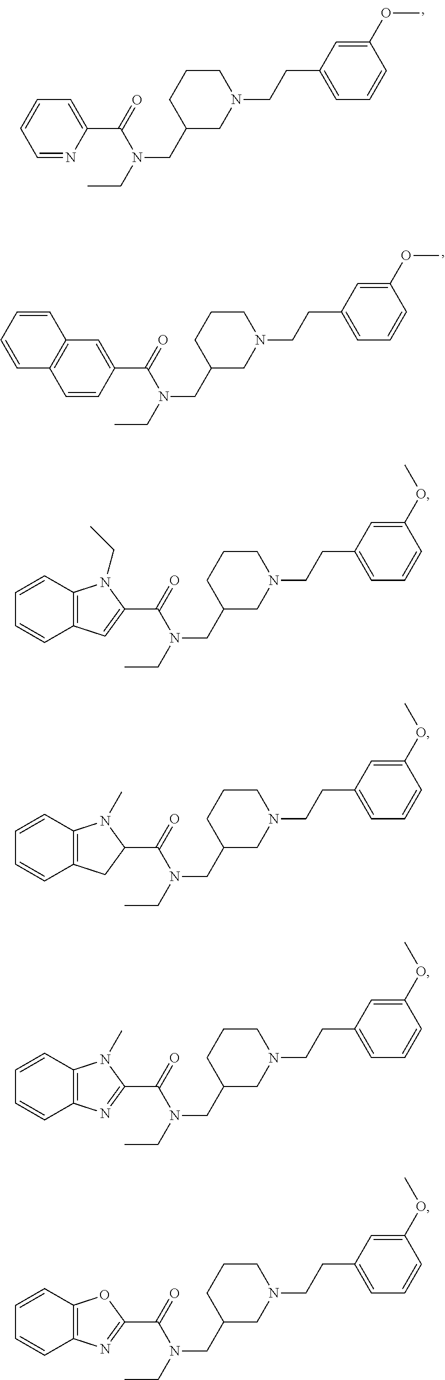 [SMILES: CCN(CC1CCCN(CCc2cccc(OC)c2)C1)C(=O)c1ccccn1 ; CCN(CC1CCCN(CCc2cccc(OC)c2)C1)C(=O)c1ccc2ccccc2c1 ; CCN(CC1CCCN(CCc2cccc(OC)c2)C1)C(=O)c1cc2ccccc2n1CC ; CCN(CC1CCCN(CCc2cccc(OC)c2)C1)C(=O)C1Cc2ccccc2N1C ; CCN(CC1CCCN(CCc2cccc(OC)c2)C1)C(=O)c1nc2ccccc2n1C ; CCN(CC1CCCN(CCc2cccc(OC)c2)C1)C(=O)c1nc2ccccc2o1]

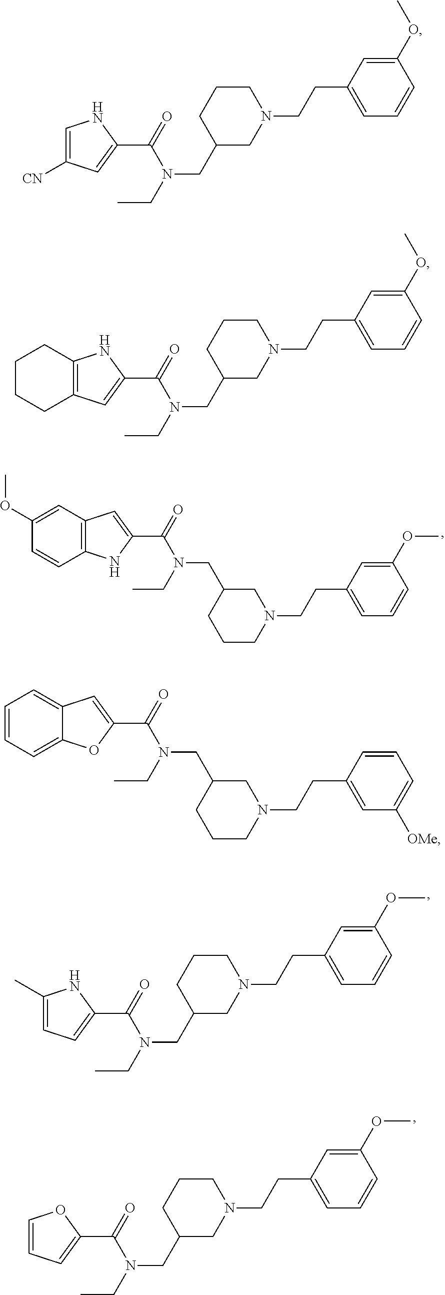

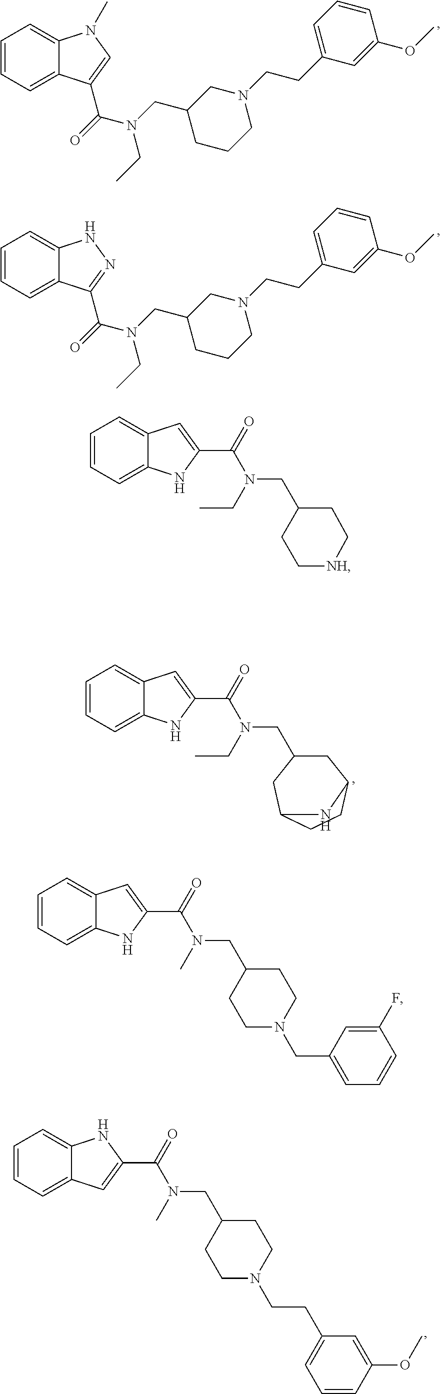

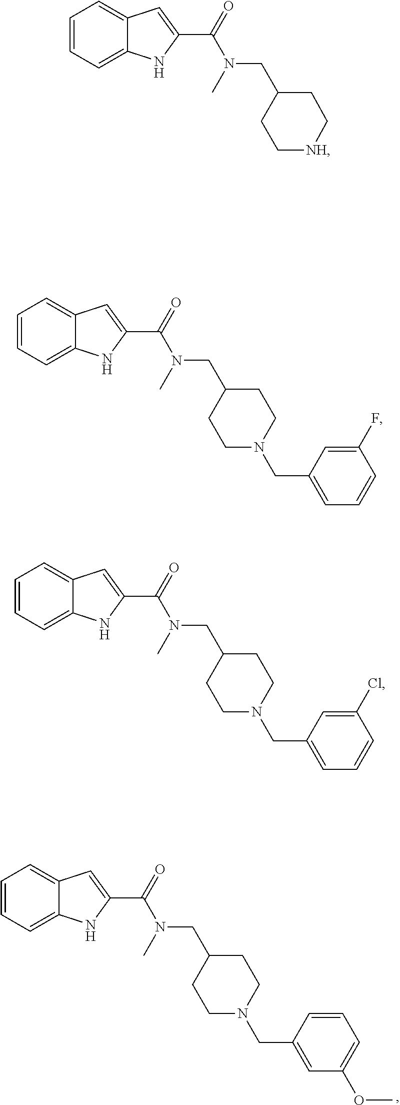 [SMILES: CN(CC1CCNCC1)C(=O)c1cc2ccccc2[nH]1 ; CN(CC1CCN(Cc2cccc(F)c2)CC1)C(=O)c1cc2ccccc2[nH]1 ; CN(CC1CCN(Cc2cccc(Cl)c2)CC1)C(=O)c1cc2ccccc2[nH]1 ; COc1cccc(CN2CCC(CN(C)C(=O)c3cc4ccccc4[nH]3)CC2)c1]

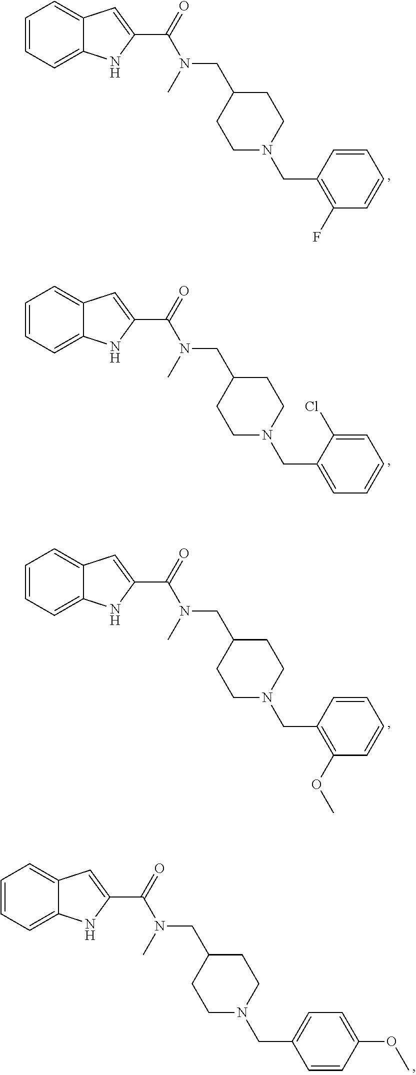 [SMILES: CN(CC1CCN(Cc2ccccc2F)CC1)C(=O)c1cc2ccccc2[nH]1 ; CN(CC1CCN(Cc2ccccc2Cl)CC1)C(=O)c1cc2ccccc2[nH]1 ; COc1ccccc1CN1CCC(CN(C)C(=O)c2cc3ccccc3[nH]2)CC1 ; COc1ccc(CN2CCC(CN(C)C(=O)c3cc4ccccc4[nH]3)CC2)cc1]

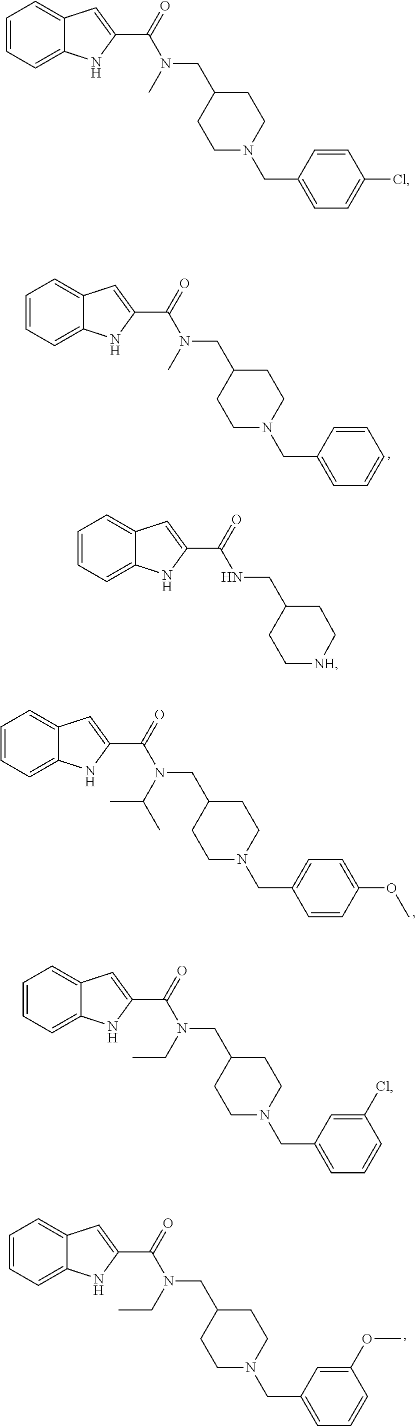 [SMILES: CN(CC1CCN(Cc2ccc(Cl)cc2)CC1)C(=O)c1cc2ccccc2[nH]1 ; CN(CC1CCN(Cc2ccccc2)CC1)C(=O)c1cc2ccccc2[nH]1 ; O=C(NCC1CCNCC1)c1cc2ccccc2[nH]1 ; COc1ccc(CN2CCC(CN(C(=O)c3cc4ccccc4[nH]3)C(C)C)CC2)cc1 ; CCN(CC1CCN(Cc2cccc(Cl)c2)CC1)C(=O)c1cc2ccccc2[nH]1 ; CCN(CC1CCN(Cc2cccc(OC)c2)CC1)C(=O)c1cc2ccccc2[nH]1]

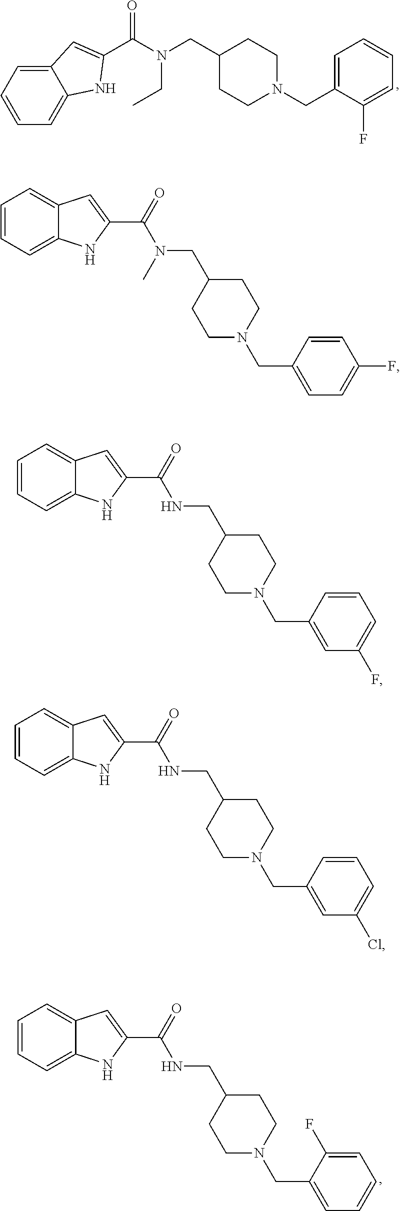 [SMILES: CCN(CC1CCN(Cc2ccccc2F)CC1)C(=O)c1cc2ccccc2[nH]1 ; CN(CC1CCN(Cc2ccc(F)cc2)CC1)C(=O)c1cc2ccccc2[nH]1 ; O=C(NCC1CCN(Cc2cccc(F)c2)CC1)c1cc2ccccc2[nH]1 ; O=C(NCC1CCN(Cc2cccc(Cl)c2)CC1)c1cc2ccccc2[nH]1 ; O=C(NCC1CCN(Cc2ccccc2F)CC1)c1cc2ccccc2[nH]1]

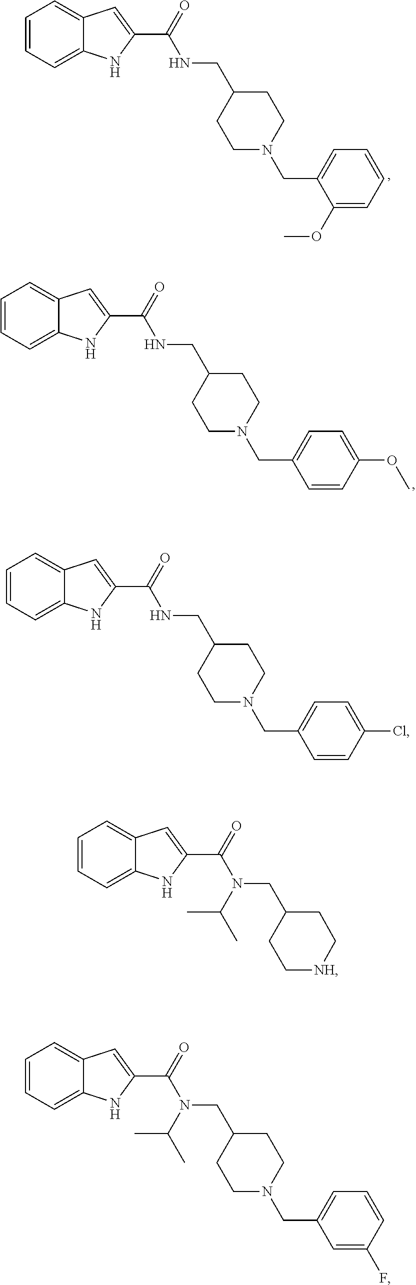 [SMILES: COc1ccccc1CN1CCC(CNC(=O)c2cc3ccccc3[nH]2)CC1 ; COc1ccc(CN2CCC(CNC(=O)c3cc4ccccc4[nH]3)CC2)cc1 ; O=C(NCC1CCN(Cc2ccc(Cl)cc2)CC1)c1cc2ccccc2[nH]1 ; CC(C)N(CC1CCNCC1)C(=O)c1cc2ccccc2[nH]1 ; CC(C)N(CC1CCN(Cc2cccc(F)c2)CC1)C(=O)c1cc2ccccc2[nH]1]

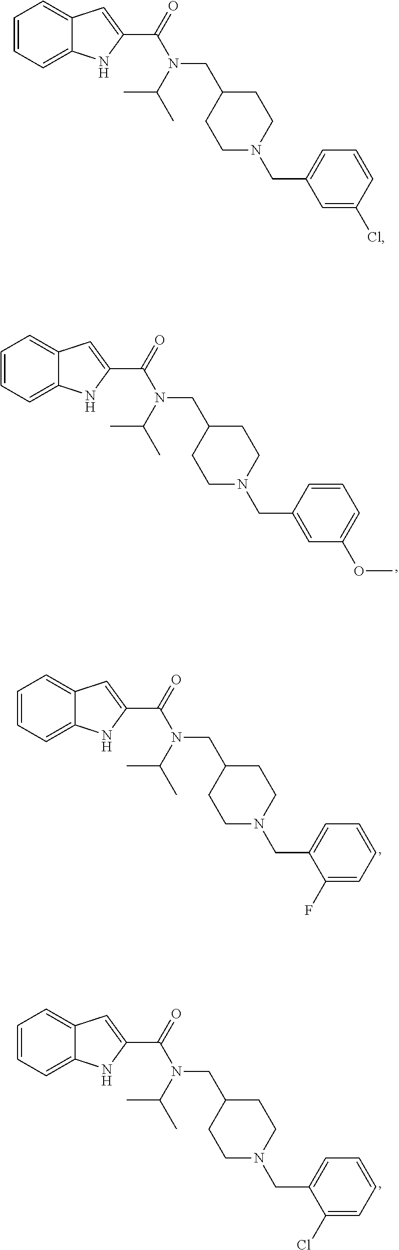 [SMILES: CC(C)N(CC1CCN(Cc2cccc(Cl)c2)CC1)C(=O)c1cc2ccccc2[nH]1 ; COc1cccc(CN2CCC(CN(C(=O)c3cc4ccccc4[nH]3)C(C)C)CC2)c1 ; CC(C)N(CC1CCN(Cc2ccccc2F)CC1)C(=O)c1cc2ccccc2[nH]1 ; CC(C)N(CC1CCN(Cc2ccccc2Cl)CC1)C(=O)c1cc2ccccc2[nH]1]

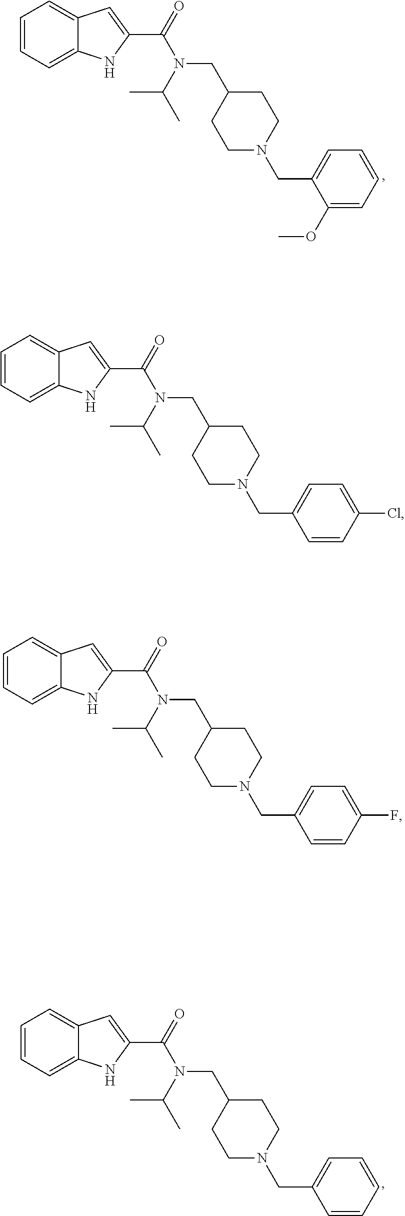 [SMILES: COc1ccccc1CN1CCC(CN(C(=O)c2cc3ccccc3[nH]2)C(C)C)CC1 ; CC(C)N(CC1CCN(Cc2ccc(Cl)cc2)CC1)C(=O)c1cc2ccccc2[nH]1 ; CC(C)N(CC1CCN(Cc2ccc(F)cc2)CC1)C(=O)c1cc2ccccc2[nH]1 ; CC(C)N(CC1CCN(Cc2ccccc2)CC1)C(=O)c1cc2ccccc2[nH]1]

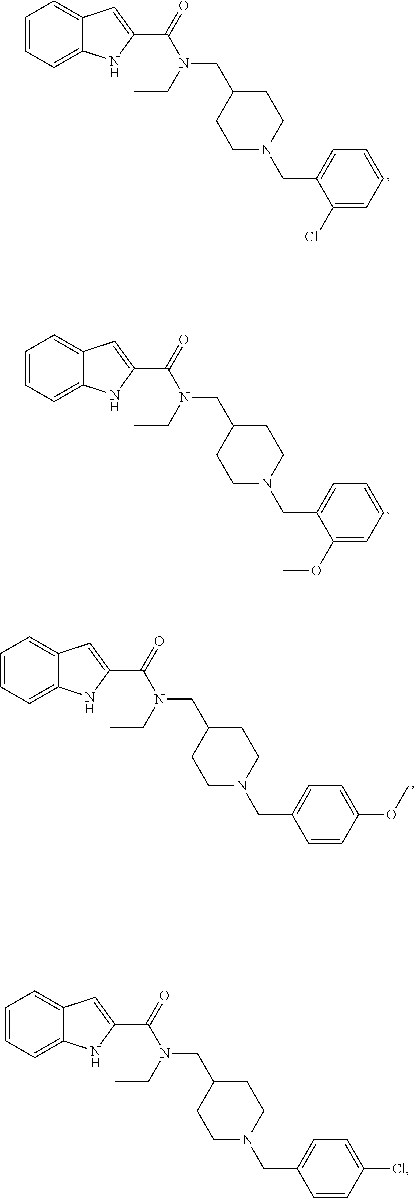 [SMILES: CCN(CC1CCN(Cc2ccccc2Cl)CC1)C(=O)c1cc2ccccc2[nH]1 ; CCN(CC1CCN(Cc2ccccc2OC)CC1)C(=O)c1cc2ccccc2[nH]1 ; CCN(CC1CCN(Cc2ccc(OC)cc2)CC1)C(=O)c1cc2ccccc2[nH]1 ; CCN(CC1CCN(Cc2ccc(Cl)cc2)CC1)C(=O)c1cc2ccccc2[nH]1]

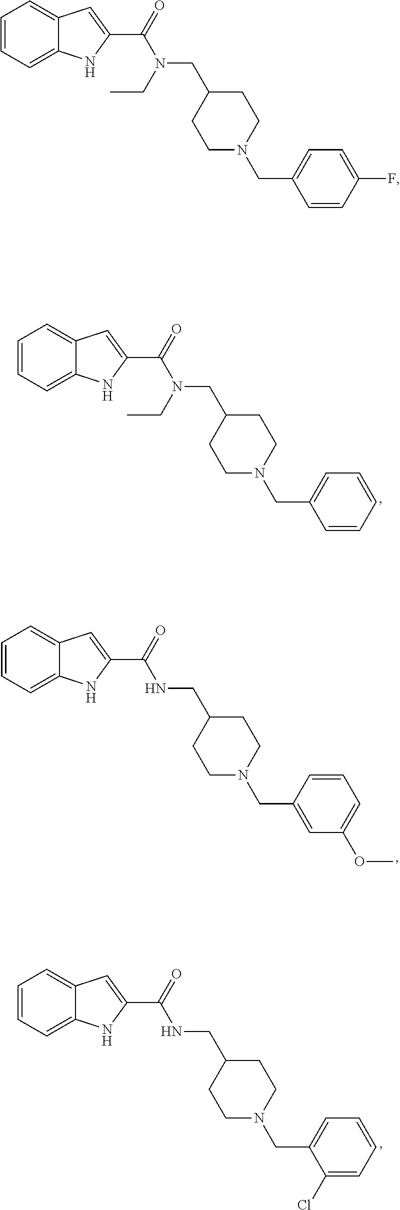 [SMILES: CCN(CC1CCN(Cc2ccc(F)cc2)CC1)C(=O)c1cc2ccccc2[nH]1 ; CCN(CC1CCN(Cc2ccccc2)CC1)C(=O)c1cc2ccccc2[nH]1 ; COc1cccc(CN2CCC(CNC(=O)c3cc4ccccc4[nH]3)CC2)c1 ; O=C(NCC1CCN(Cc2ccccc2Cl)CC1)c1cc2ccccc2[nH]1]

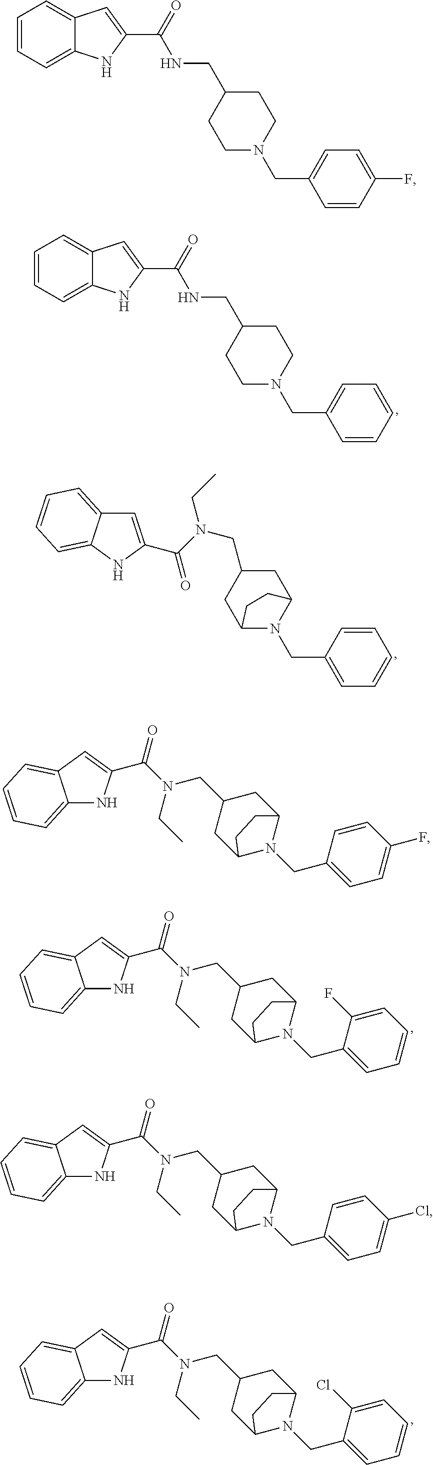

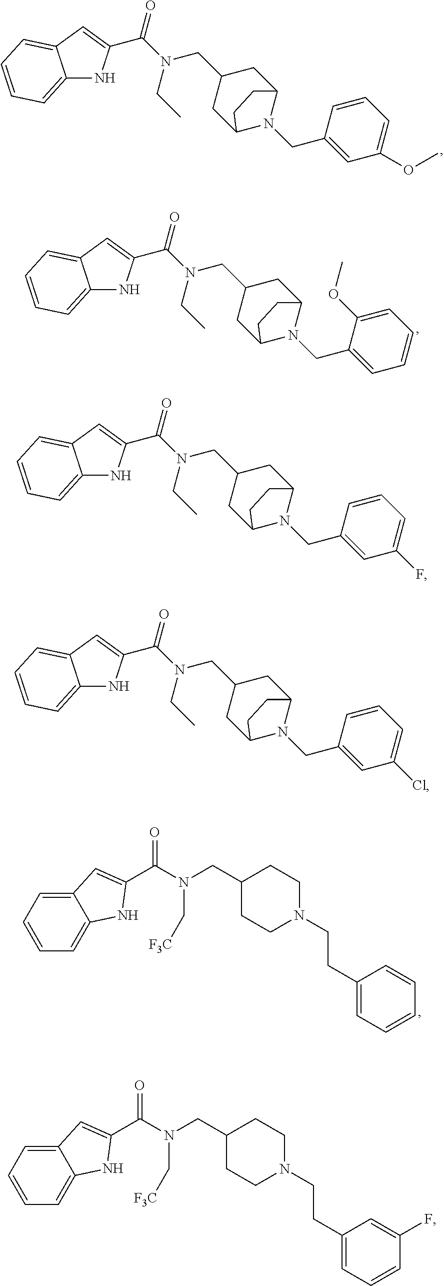 [SMILES: CCN(CC1CC2CCC(C1)N2Cc1cccc(OC)c1)C(=O)c1cc2ccccc2[nH]1 ; CCN(CC1CC2CCC(C1)N2Cc1ccccc1OC)C(=O)c1cc2ccccc2[nH]1 ; CCN(CC1CC2CCC(C1)N2Cc1cccc(F)c1)C(=O)c1cc2ccccc2[nH]1 ; CCN(CC1CC2CCC(C1)N2Cc1cccc(Cl)c1)C(=O)c1cc2ccccc2[nH]1 ; O=C(c1cc2ccccc2[nH]1)N(CC1CCN(CCc2ccccc2)CC1)CC(F)(F)F ; O=C(c1cc2ccccc2[nH]1)N(CC1CCN(CCc2cccc(F)c2)CC1)CC(F)(F)F]

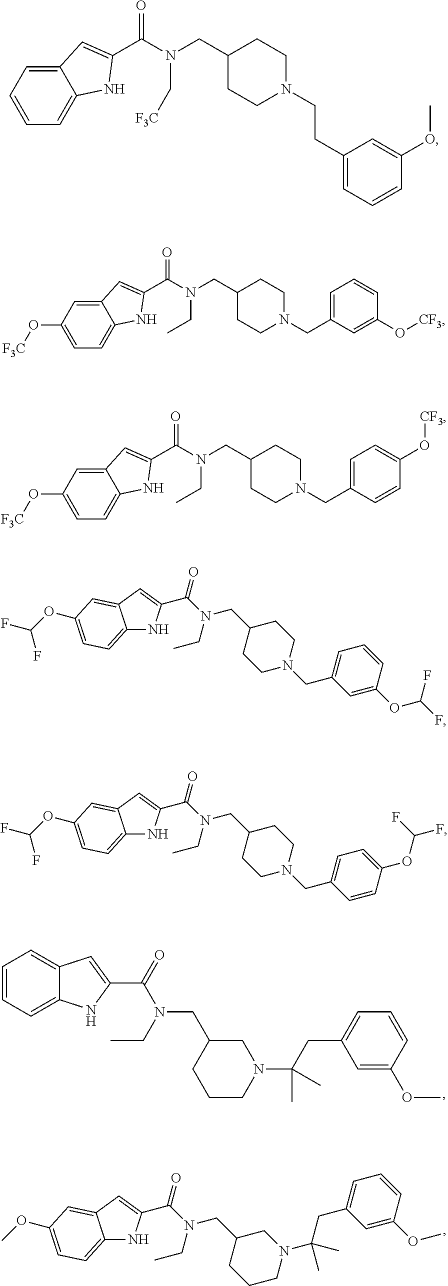

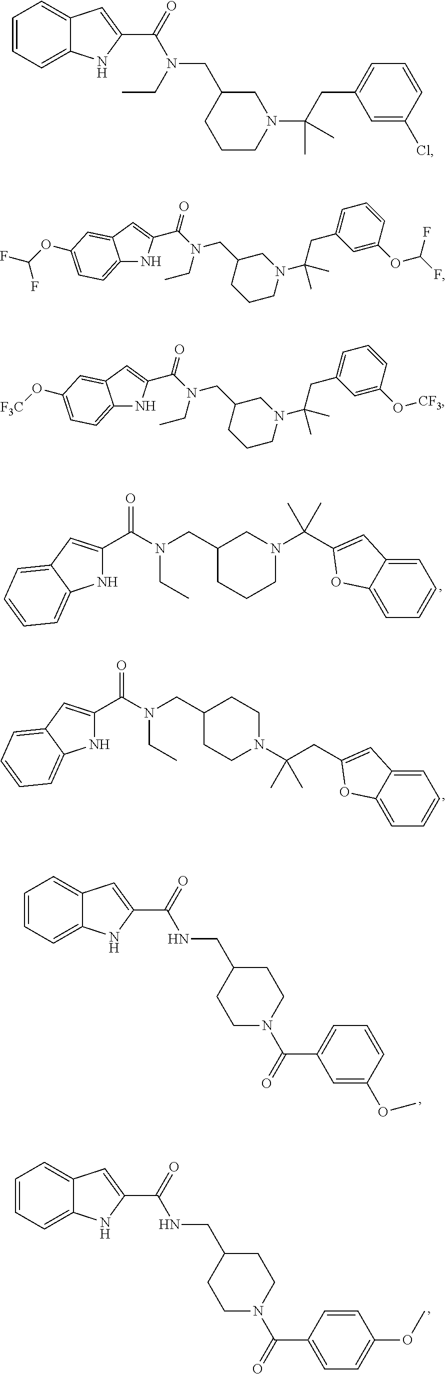

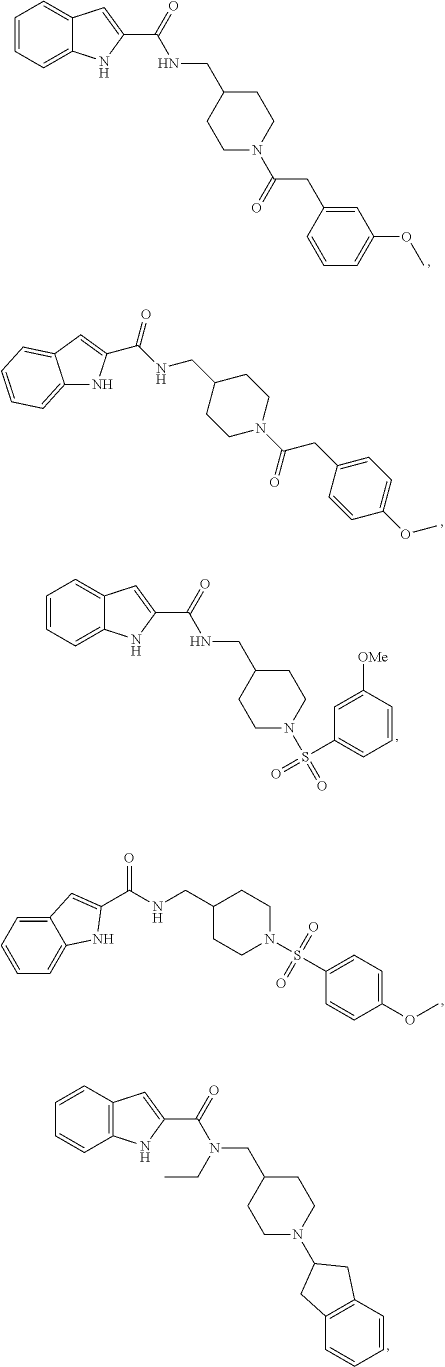 [SMILES: COc1cccc(CC(=O)N2CCC(CNC(=O)c3cc4ccccc4[nH]3)CC2)c1 ; COc1ccc(CC(=O)N2CCC(CNC(=O)c3cc4ccccc4[nH]3)CC2)cc1 ; COc1cccc(S(=O)(=O)N2CCC(CNC(=O)c3cc4ccccc4[nH]3)CC2)c1 ; COc1ccc(S(=O)(=O)N2CCC(CNC(=O)c3cc4ccccc4[nH]3)CC2)cc1 ; CCN(CC1CCN(C2Cc3ccccc3C2)CC1)C(=O)c1cc2ccccc2[nH]1]

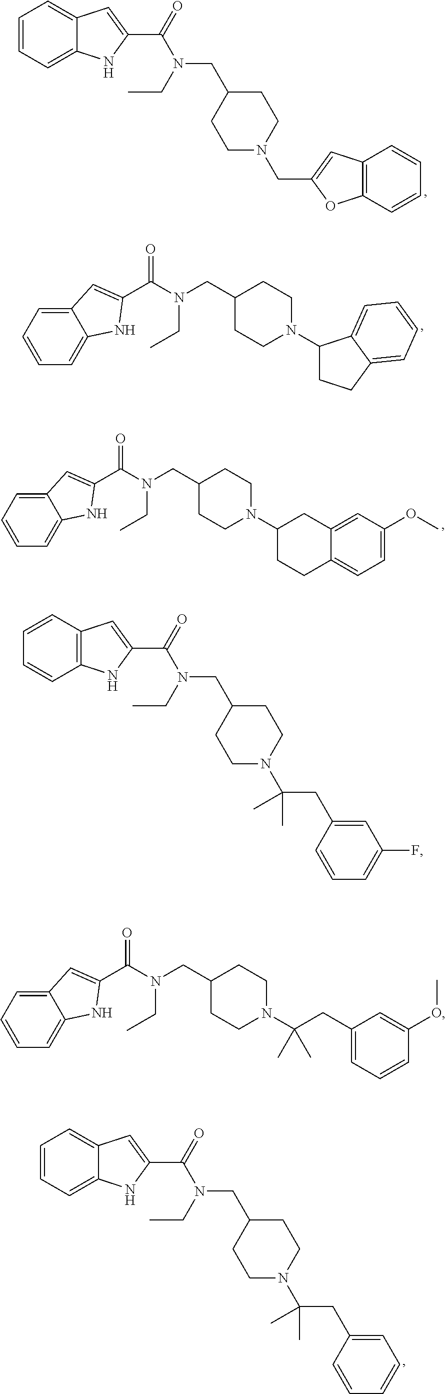 [SMILES: CCN(CC1CCN(Cc2cc3ccccc3o2)CC1)C(=O)c1cc2ccccc2[nH]1 ; CCN(CC1CCN(C2CCc3ccccc32)CC1)C(=O)c1cc2ccccc2[nH]1 ; CCN(CC1CCN(C2CCc3ccc(OC)cc3C2)CC1)C(=O)c1cc2ccccc2[nH]1 ; CCN(CC1CCN(C(C)(C)Cc2cccc(F)c2)CC1)C(=O)c1cc2ccccc2[nH]1 ; CCN(CC1CCN(C(C)(C)Cc2cccc(OC)c2)CC1)C(=O)c1cc2ccccc2[nH]1 ; CCN(CC1CCN(C(C)(C)Cc2ccccc2)CC1)C(=O)c1cc2ccccc2[nH]1]

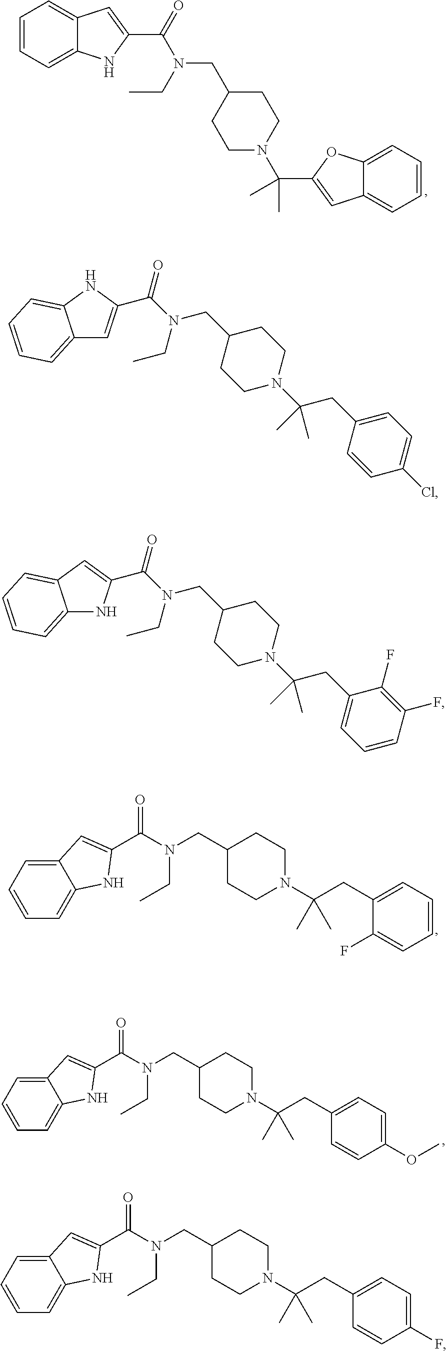 [SMILES: CCN(CC1CCN(C(C)(C)c2cc3ccccc3o2)CC1)C(=O)c1cc2ccccc2[nH]1 ; CCN(CC1CCN(C(C)(C)Cc2ccc(Cl)cc2)CC1)C(=O)c1cc2ccccc2[nH]1 ; CCN(CC1CCN(C(C)(C)Cc2cccc(F)c2F)CC1)C(=O)c1cc2ccccc2[nH]1 ; CCN(CC1CCN(C(C)(C)Cc2ccccc2F)CC1)C(=O)c1cc2ccccc2[nH]1 ; CCN(CC1CCN(C(C)(C)Cc2ccc(OC)cc2)CC1)C(=O)c1cc2ccccc2[nH]1 ; CCN(CC1CCN(C(C)(C)Cc2ccc(F)cc2)CC1)C(=O)c1cc2ccccc2[nH]1]

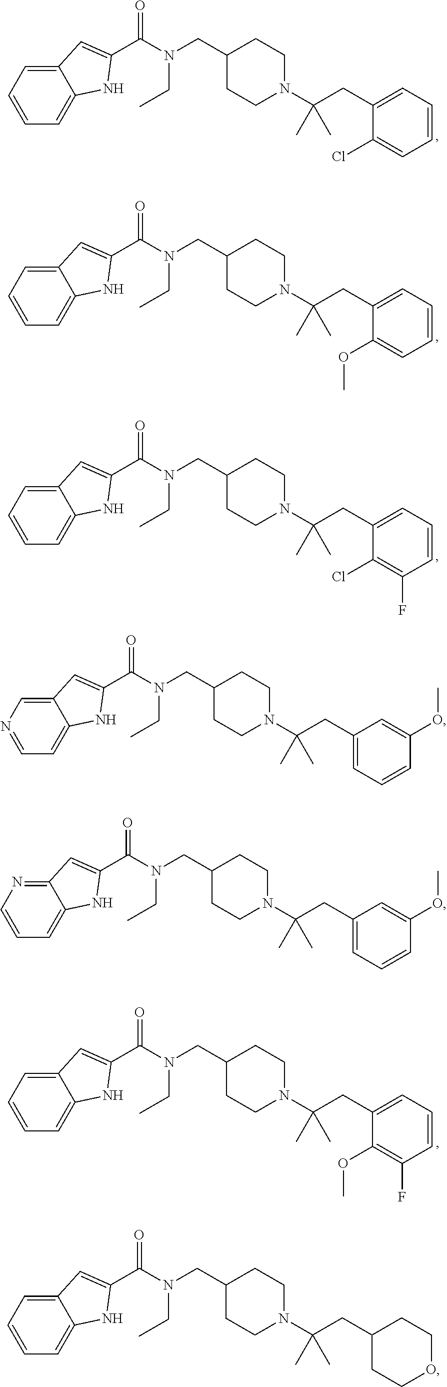

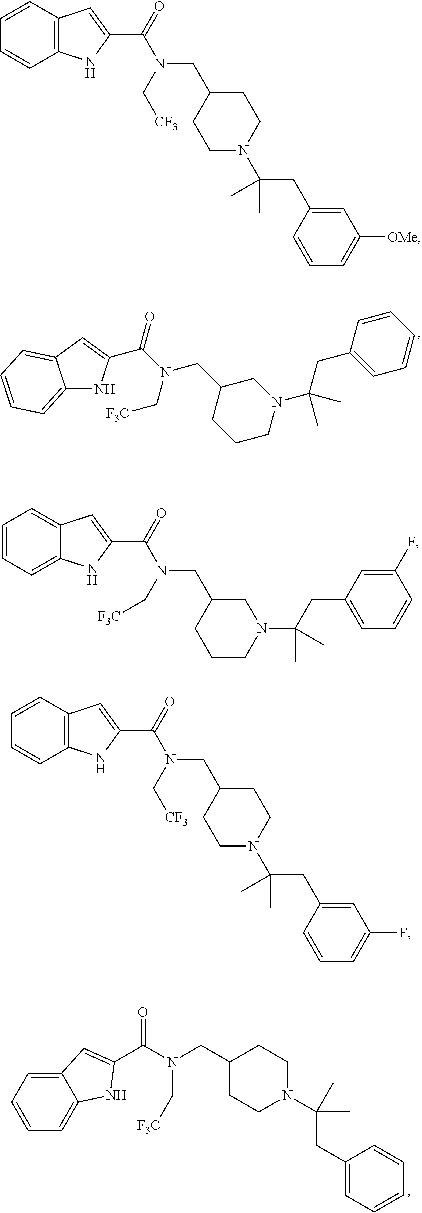 [SMILES: COc1cccc(CC(C)(C)N2CCC(CN(CC(F)(F)F)C(=O)c3cc4ccccc4[nH]3)CC2)c1 ; CC(C)(Cc1ccccc1)N1CCCC(CN(CC(F)(F)F)C(=O)c2cc3ccccc3[nH]2)C1 ; CC(C)(Cc1cccc(F)c1)N1CCCC(CN(CC(F)(F)F)C(=O)c2cc3ccccc3[nH]2)C1 ; CC(C)(Cc1cccc(F)c1)N1CCC(CN(CC(F)(F)F)C(=O)c2cc3ccccc3[nH]2)CC1 ; CC(C)(Cc1ccccc1)N1CCC(CN(CC(F)(F)F)C(=O)c2cc3ccccc3[nH]2)CC1]

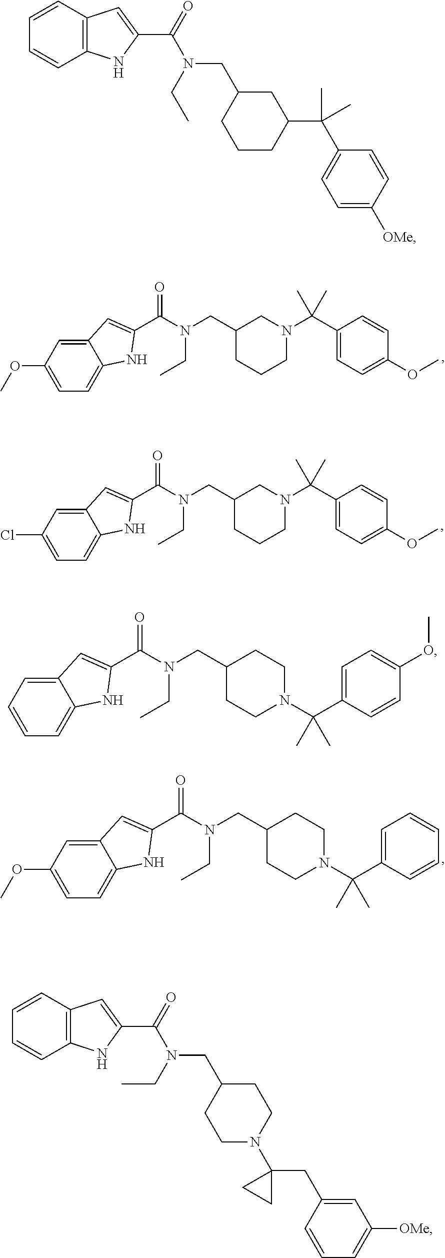

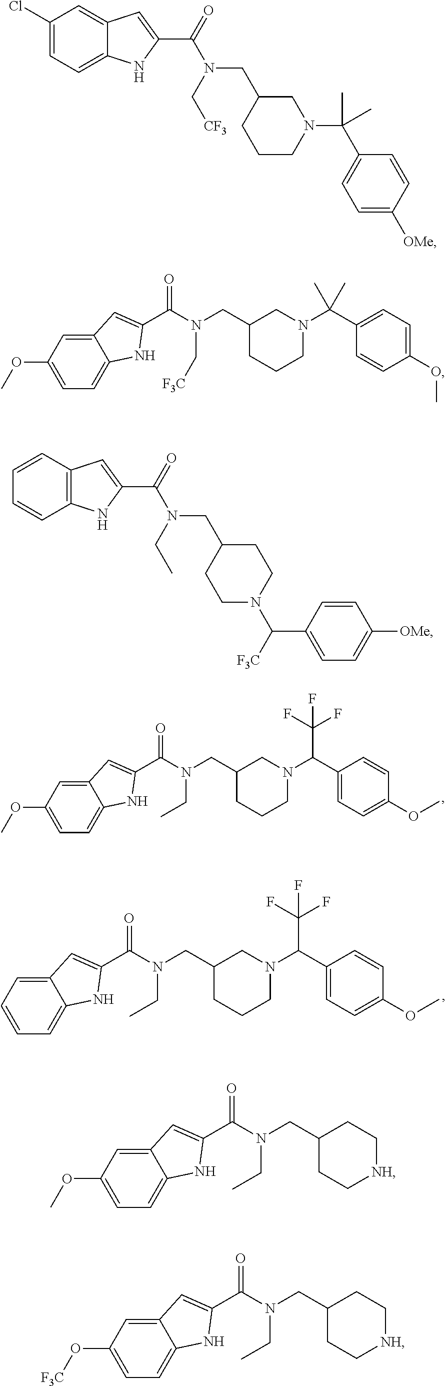

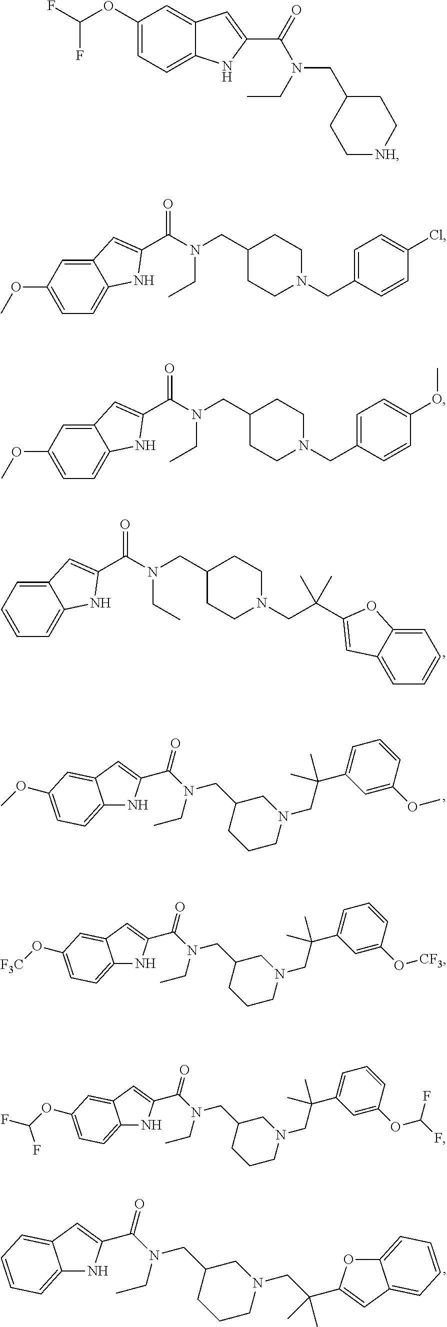

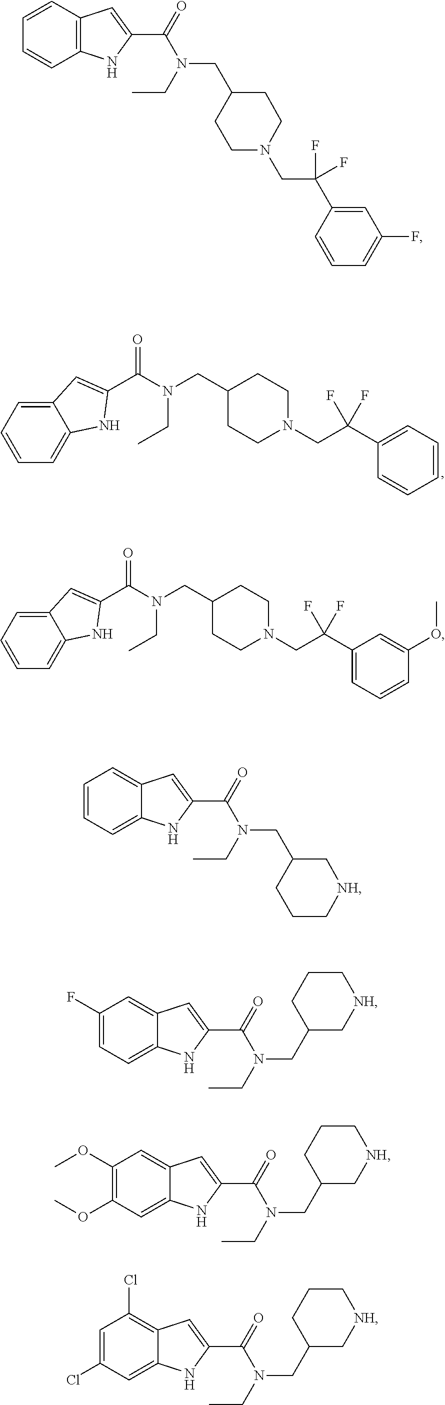

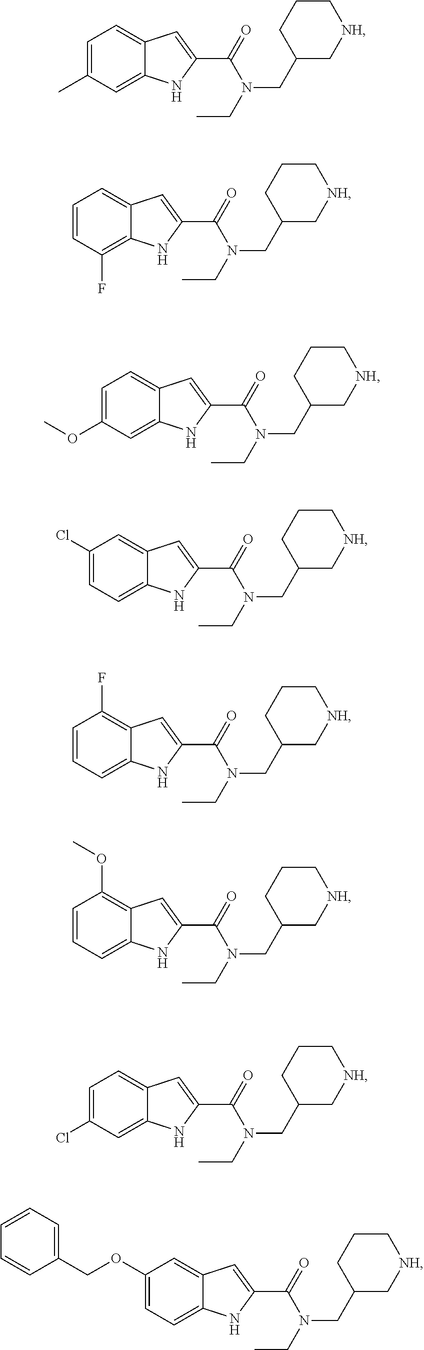

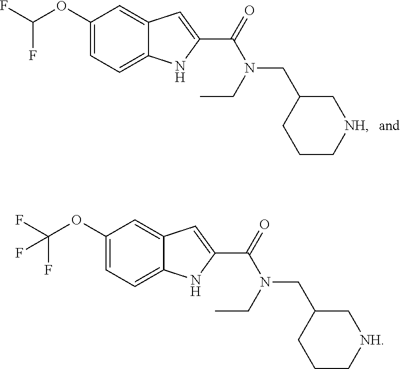 [SMILES: CCN(CC1CCCNC1)C(=O)c1cc2cc(OC(F)F)ccc2[nH]1 ; CCN(CC1CCCNC1)C(=O)c1cc2cc(OC(F)(F)F)ccc2[nH]1]

53. A pharmaceutical composition comprising at least one compound according claim 1 or a pharmaceutically acceptable salt thereof in a mixture with a pharmaceutically acceptable excipient, diluent or carrier.

54. A method for modulating stress granules, the method comprising use of a compound of Formula (I) or a pharmaceutically acceptable salt thereof according to claim 1.

55. The method of claim 54, wherein stress granule formation is inhibited.

56. The method of claim 54, wherein the stress granule is disaggregated.

57. The method of claim 54, wherein stress granule formation is stimulated.

58. The method of claim 54, wherein the stress granule comprises tar DNA binding protein-43 (TDP-43), T-cell intracellular antigen 1 (TIA-1), TIA1 cytotoxic granule-associated RNA binding protein-like 1 (TIAR), GTPase activating protein binding protein 1 (G3BP-1), GTPase activating protein binding protein 2 (G3BP-2), tris tetraprolin (TTP), fused in sarcoma (FUS), or fragile X mental retardation protein (FMRP).

59. A method for modulating TDP-43 inclusion formation, the method comprising use of a compound of Formula (I) or a pharmaceutically acceptable salt thereof according to claim 1.

60. The method of claim 59, wherein TDP-43 inclusion formation is inhibited.

61. The method of claim 59, wherein the TDP-43 inclusion is disaggregated.

62. The method of claim 59, wherein TDP-43 inclusion formation is stimulated.

63. The method of claim 54, wherein the composition is administered to a subject suffering from a neurodegenerative disease or disorder, a musculoskeletal disease or disorder, a cancer, an ophthalmological disease or disorder, and/or a viral infection.

64-72. (canceled)

73. The method of claim 63, further comprising the step of diagnosing the subject with the neurodegenerative disease or disorder, musculoskeletal disease or disorder, cancer, ophthalmological disease or disorder, or viral infection prior to onset of said administration.

74. The method of claim 63, wherein pathology of said neurodegenerative disease or disorder, said musculoskeletal disease or disorder, said cancer, said ophthalmological disease or disorder, and said viral infection comprises stress granules.

75. The method of claim 63, wherein pathology of said neurodegenerative disease, said musculoskeletal disease or disorder, said cancer, said ophthalmological disease or disorder, and said viral infection comprises TDP-43 inclusions.
